SEQUENCE LISTING (828 sequences)

SEQ ID NO: 1 - protein, 109 aa, synthetic construct
  REGION 1..109: note = Synthetic Construct
  VARIANT 14: note = Xaa is Phe or Tyr
  VARIANT 15: note = Xaa is Phe or Tyr
  VARIANT 20: note = Xaa is Glu or Ala
  VARIANT 21: note = Xaa is Lys or Leu
  VARIANT 22: note = Xaa is Asp or Glu
  VARIANT 23: note = Xaa is Arg or Ala
  VARIANT 31: note = Xaa is Pro or Arg
  VARIANT 33: note = Xaa is Tyr or Glu
  VARIANT 35: note = Xaa is Asp or Glu
  VARIANT 36: note = Xaa is Lys or Gln
  VARIANT 37: note = Xaa is Asp or Ala
  VARIANT 38: note = Xaa is Lys or Ala
  VARIANT 39: note = Xaa is Arg or Ala
  VARIANT 40: note = Xaa is Arg or Leu
  VARIANT 43: note = Xaa is Phe or Tyr
  VARIANT 47: note = Xaa is Lys, Arg, or Ala
  VARIANT 57: note = Xaa is Lys, Ala, Tyr, Phe, or Ile
  VARIANT 58: note = Xaa is Gln or Lys
  VARIANT 61: note = Xaa is Trp or Ala
  VARIANT 62: note = Xaa is Leu or Ala
  VARIANT 63: note = Xaa is Asp, Lys, Arg, Ala, Phe, Gly, Met, Asn, or Ile
  VARIANT 65: note = Xaa is Ile, Phe, or Ala
  VARIANT 76: note = Xaa is Lys or Thr
  VARIANT 77: note = Xaa is Lys or Glu
  VARIANT 78: note = Xaa is Asp or Glu
  VARIANT 79: note = Xaa is Ser or Asn
  VARIANT 81: note = Xaa is Glu or Gln
GAILGRSETQECLXXNANWXXXXTNQTGVEXCXGXXXXXXHCXATWXNISGSIEIVXXGCXXXDXNCYDRTDCVEXXXXPXVYFCCCEGNMCNEKFSYFPEMEVTQPTS

SEQ ID NO: 2 - protein, 109 aa, synthetic construct
  REGION 1..109: note = Synthetic Construct
  VARIANT 15: note = Xaa is Phe or Tyr
  VARIANT 20: note = Xaa is Glu or Ala
  VARIANT 21: note = Xaa is Lys or Leu
  VARIANT 22: note = Xaa is Asp or Glu
  VARIANT 23: note = Xaa is Arg or Ala
  VARIANT 31: note = Xaa is Pro or Arg
  VARIANT 33: note = Xaa is Tyr or Glu
  VARIANT 35: note = Xaa is Asp or Glu
  VARIANT 37: note = Xaa is Asp or Ala
  VARIANT 38: note = Xaa is Lys or Ala
  VARIANT 39: note = Xaa is Arg or Ala
  VARIANT 40: note = Xaa is Arg or Leu
  VARIANT 43: note = Xaa is Phe or Tyr
  VARIANT 47: note = Xaa is Lys, Arg, or Ala
  VARIANT 57: note = Xaa is Lys, Ala, Tyr, Phe, or Ile
  VARIANT 58: note = Xaa is Gln or Lys
  VARIANT 61: note = Xaa is Trp or Ala
  VARIANT 62: note = Xaa is Leu or Ala
  VARIANT 63: note = Xaa is Asp, Lys, Arg, Ala, Phe, Gly, Met, Asn, or Ile
  VARIANT 65: note = Xaa is Ile, Phe, or Ala
  VARIANT 76: note = Xaa is Lys or Thr
  VARIANT 77: note = Xaa is Lys or Glu
  VARIANT 78: note = Xaa is Asp or Glu
  VARIANT 79: note = Xaa is Ser or Asn
  VARIANT 81: note = Xaa is Glu or Gln
GAILGRSETQECLFXNANWXXXXTNQTGVEXCXGXKXXXXHCXATWXNISGSIEIVXXGCXXXDXNCYDRTDCVEXXXXPXVYFCCCEGNMCNEKFSYFPEMEVTQPTS

SEQ ID NO: 3 - protein, 109 aa, synthetic construct
  REGION 1..109: note = Synthetic Construct
  VARIANT 15: note = Xaa is Phe or Tyr
  VARIANT 21: note = Xaa is Lys or Leu
  VARIANT 22: note = Xaa is Asp or Glu
  VARIANT 31: note = Xaa is Pro or Arg
  VARIANT 33: note = Xaa is Tyr or Glu
  VARIANT 35: note = Xaa is Asp or Glu
  VARIANT 40: note = Xaa is Arg or Leu
  VARIANT 43: note = Xaa is Phe or Tyr
  VARIANT 47: note = Xaa is Lys, Arg, or Ala
  VARIANT 58: note = Xaa is Gln or Lys
  VARIANT 65: note = Xaa is Ile, Phe, or Ala
  VARIANT 76: note = Xaa is Lys or Thr
  VARIANT 77: note = Xaa is Lys or Glu
  VARIANT 78: note = Xaa is Asp or Glu
  VARIANT 79: note = Xaa is Ser or Asn
  VARIANT 81: note = Xaa is Glu or Gln
GAILGRSETQECLFXNANWEXXRTNQTGVEXCXGXKDKRXHCXATWXNISGSIEIVKXGCWLDDXNCYDRTDCVEXXXXPXVYFCCCEGNMCNEKFSYFPEMEVTQPTS

SEQ ID NO: 4 - protein, 109 aa, synthetic construct
  REGION 1..109: note = Synthetic Construct
  VARIANT 15: note = Xaa is Phe or Tyr
  VARIANT 21: note = Xaa is Lys or Leu
  VARIANT 31: note = Xaa is Pro or Arg
  VARIANT 33: note = Xaa is Tyr or Glu
  VARIANT 35: note = Xaa is Asp or Glu
  VARIANT 40: note = Xaa is Arg or Leu
  VARIANT 43: note = Xaa is Phe or Tyr
  VARIANT 47: note = Xaa is Lys, Arg, or Ala
  VARIANT 58: note = Xaa is Gln or Lys
  VARIANT 65: note = Xaa is Ile, Phe, or Ala
  VARIANT 76: note = Xaa is Lys or Thr
  VARIANT 78: note = Xaa is Asp or Glu
  VARIANT 79: note = Xaa is Ser or Asn
  VARIANT 81: note = Xaa is Glu or Gln
GAILGRSETQECLFXNANWEXDRTNQTGVEXCXGXKDKRXHCXATWXNISGSIEIVKXGCWLDDXNCYDRTDCVEXKXXPXVYFCCCEGNMCNEKFSYFPEMEVTQPTS

SEQ ID NO: 5 - protein, 109 aa, synthetic construct
  REGION 1..109: note = Synthetic Construct
  VARIANT 15: note = Xaa is Phe or Tyr
  VARIANT 21: note = Xaa is Lys or Leu
  VARIANT 33: note = Xaa is Tyr or Glu
  VARIANT 35: note = Xaa is Asp or Glu
  VARIANT 40: note = Xaa is Arg or Leu
  VARIANT 58: note = Xaa is Gln or Lys
  VARIANT 76: note = Xaa is Lys or Thr
  VARIANT 78: note = Xaa is Asp or Glu
  VARIANT 79: note = Xaa is Ser or Asn
  VARIANT 81: note = Xaa is Glu or Gln
GAILGRSETQECLFXNANWEXDRTNQTGVEPCXGXKDKRXHCFATWKNISGSIEIVKXGCWLDDINCYDRTDCVEXKXXPXVYFCCCEGNMCNEKFSYFPEMEVTQPTS

SEQ ID NO: 6 - protein, 109 aa, synthetic construct
  REGION 1..109: note = Synthetic Construct
GAILGRSETQECLFYNANWELDRTNQTGVEPCEGEKDKRLHCFATWKNISGSIEIVKKGCWLDDINCYDRTDCVETKENPQVYFCCCEGNMCNEKFSYFPEMEVTQPTS

SEQ ID NO: 7 - protein, 109 aa, synthetic construct
  REGION 1..109: note = Synthetic Construct
GAILGRSETQECLFYNANWELERTNQTGVEPCEGEKDKRLHCFATWKNISGSIEIVKKGCWLDDINCYDRTDCVETKENPQVYFCCCEGNMCNEKFSYFPEMEVTQPTS

SEQ ID NO: 8 - protein, 109 aa, synthetic construct
  REGION 1..109: note = Synthetic Construct
GAILGRSETQECLFYNANWELDRTNQTGVERCEGEKDKRLHCFATWKNISGSIEIVKKGCWLDDINCYDRTDCVETKENPQVYFCCCEGNMCNEKFSYFPEMEVTQPTS

SEQ ID NO: 9 - protein, 109 aa, synthetic construct
  REGION 1..109: note = Synthetic Construct
GAILGRSETQECLFYNANWELDRTNQTGVEPCEGEKDKRLHCYATWKNISGSIEIVKKGCWLDDINCYDRTDCVETKENPQVYFCCCEGNMCNEKFSYFPEMEVTQPTS

SEQ ID NO: 10 - protein, 109 aa, synthetic construct
  REGION 1..109: note = Synthetic Construct
GAILGRSETQECLFYNANWELDRTNQTGVEPCEGEKDKRLHCFATWRNISGSIEIVKKGCWLDDINCYDRTDCVETKENPQVYFCCCEGNMCNEKFSYFPEMEVTQPTS

SEQ ID NO: 11 - protein, 109 aa, synthetic construct
  REGION 1..109: note = Synthetic Construct
GAILGRSETQECLFYNANWELDRTNQTGVEPCEGEKDKRLHCFATWKNISGSIEIVKKGCWLDDFNCYDRTDCVETKENPQVYFCCCEGNMCNEKFSYFPEMEVTQPTS

SEQ ID NO: 12 - protein, 109 aa, synthetic construct
  REGION 1..109: note = Synthetic Construct
GAILGRSETQECLFYNANWELDRTNQTGVEPCEGEKDKRLHCFATWKNISGSIEIVKKGCWLDDINCYDRTDCVETEENPQVYFCCCEGNMCNEKFSYFPEMEVTQPTS

SEQ ID NO: 13 - protein, 109 aa, synthetic construct
  REGION 1..109: note = Synthetic Construct
GAILGRSETQECLFYNANWELERTNQTGVERCEGEKDKRLHCFATWKNISGSIEIVKKGCWLDDINCYDRTDCVETKENPQVYFCCCEGNMCNEKFSYFPEMEVTQPTS

SEQ ID NO: 14 - protein, 109 aa, synthetic construct
  REGION 1..109: note = Synthetic Construct
GAILGRSETQECLFYNANWELERTNQTGVEPCEGEKDKRLHCYATWKNISGSIEIVKKGCWLDDINCYDRTDCVETKENPQVYFCCCEGNMCNEKFSYFPEMEVTQPTS

SEQ ID NO: 15 - protein, 109 aa, synthetic construct
  REGION 1..109: note = Synthetic Construct
GAILGRSETQECLFYNANWELERTNQTGVEPCEGEKDKRLHCFATWRNISGSIEIVKKGCWLDDINCYDRTDCVETKENPQVYFCCCEGNMCNEKFSYFPEMEVTQPTS

SEQ ID NO: 16 - protein, 109 aa, synthetic construct
  REGION 1..109: note = Synthetic Construct
GAILGRSETQECLFYNANWELERTNQTGVEPCEGEKDKRLHCFATWKNISGSIEIVKKGCWLDDFNCYDRTDCVETKENPQVYFCCCEGNMCNEKFSYFPEMEVTQPTS

SEQ ID NO: 17 - protein, 109 aa, synthetic construct
  REGION 1..109: note = Synthetic Construct
GAILGRSETQECLFYNANWELERTNQTGVEPCEGEKDKRLHCFATWKNISGSIEIVKKGCWLDDINCYDRTDCVETEENPQVYFCCCEGNMCNEKFSYFPEMEVTQPTS

SEQ ID NO: 18 - protein, 109 aa, synthetic construct
  REGION 1..109: note = Synthetic Construct
GAILGRSETQECLFYNANWELDRTNQTGVERCEGEKDKRLHCYATWKNISGSIEIVKKGCWLDDINCYDRTDCVETKENPQVYFCCCEGNMCNEKFSYFPEMEVTQPTS

SEQ ID NO: 19 - protein, 109 aa, synthetic construct
  REGION 1..109: note = Synthetic Construct
GAILGRSETQECLFYNANWELDRTNQTGVERCEGEKDKRLHCFATWRNISGSIEIVKKGCWLDDINCYDRTDCVETKENPQVYFCCCEGNMCNEKFSYFPEMEVTQPTS

SEQ ID NO: 20 - protein, 109 aa, synthetic construct
  REGION 1..109: note = Synthetic Construct
GAILGRSETQECLFYNANWELDRTNQTGVERCEGEKDKRLHCFATWKNISGSIEIVKKGCWLDDFNCYDRTDCVETKENPQVYFCCCEGNMCNEKFSYFPEMEVTQPTS

SEQ ID NO: 21 - protein, 109 aa, synthetic construct
  REGION 1..109: note = Synthetic Construct
GAILGRSETQECLFYNANWELDRTNQTGVERCEGEKDKRLHCFATWKNISGSIEIVKKGCWLDDINCYDRTDCVETEENPQVYFCCCEGNMCNEKFSYFPEMEVTQPTS

SEQ ID NO: 22 - protein, 109 aa, synthetic construct
  REGION 1..109: note = Synthetic Construct
GAILGRSETQECLFYNANWELDRTNQTGVEPCEGEKDKRLHCYATWRNISGSIEIVKKGCWLDDINCYDRTDCVETKENPQVYFCCCEGNMCNEKFSYFPEMEVTQPTS

SEQ ID NO: 23 - protein, 109 aa, synthetic construct
  REGION 1..109: note = Synthetic Construct
GAILGRSETQECLFYNANWELDRTNQTGVEPCEGEKDKRLHCYATWKNISGSIEIVKKGCWLDDFNCYDRTDCVETKENPQVYFCCCEGNMCNEKFSYFPEMEVTQPTS

SEQ ID NO: 24 - protein, 109 aa, synthetic construct
  REGION 1..109: note = Synthetic Construct
GAILGRSETQECLFYNANWELDRTNQTGVEPCEGEKDKRLHCYATWKNISGSIEIVKKGCWLDDINCYDRTDCVETEENPQVYFCCCEGNMCNEKFSYFPEMEVTQPTS

SEQ ID NO: 25 - protein, 109 aa, synthetic construct
  REGION 1..109: note = Synthetic Construct
GAILGRSETQECLFYNANWELDRTNQTGVEPCEGEKDKRLHCFATWRNISGSIEIVKKGCWLDDFNCYDRTDCVETKENPQVYFCCCEGNMCNEKFSYFPEMEVTQPTS

SEQ ID NO: 26 - protein, 109 aa, synthetic construct
  REGION 1..109: note = Synthetic Construct
GAILGRSETQECLFYNANWELDRTNQTGVEPCEGEKDKRLHCFATWRNISGSIEIVKKGCWLDDINCYDRTDCVETEENPQVYFCCCEGNMCNEKFSYFPEMEVTQPTS

SEQ ID NO: 27 - protein, 109 aa, synthetic construct
  REGION 1..109: note = Synthetic Construct
GAILGRSETQECLFYNANWELDRTNQTGVEPCEGEKDKRLHCFATWKNISGSIEIVKKGCWLDDFNCYDRTDCVETEENPQVYFCCCEGNMCNEKFSYFPEMEVTQPTS

SEQ ID NO: 28 - protein, 109 aa, synthetic construct
  REGION 1..109: note = Synthetic Construct
GAILGRSETQECLFYNANWELERTNQTGVERCEGEKDKRLHCYATWKNISGSIEIVKKGCWLDDINCYDRTDCVETKENPQVYFCCCEGNMCNEKFSYFPEMEVTQPTS

SEQ ID NO: 29 - protein, 109 aa, synthetic construct
  REGION 1..109: note = Synthetic Construct
GAILGRSETQECLFYNANWELERTNQTGVERCEGEKDKRLHCFATWRNISGSIEIVKKGCWLDDINCYDRTDCVETKENPQVYFCCCEGNMCNEKFSYFPEMEVTQPTS

SEQ ID NO: 30 - protein, 109 aa, synthetic construct
  REGION 1..109: note = Synthetic Construct
GAILGRSETQECLFYNANWELERTNQTGVERCEGEKDKRLHCFATWKNISGSIEIVKKGCWLDDFNCYDRTDCVETKENPQVYFCCCEGNMCNEKFSYFPEMEVTQPTS

SEQ ID NO: 31 - protein, 109 aa, synthetic construct
  REGION 1..109: note = Synthetic Construct
GAILGRSETQECLFYNANWELERTNQTGVERCEGEKDKRLHCFATWKNISGSIEIVKKGCWLDDINCYDRTDCVETEENPQVYFCCCEGNMCNEKFSYFPEMEVTQPTS

SEQ ID NO: 32 - protein, 109 aa, synthetic construct
  REGION 1..109: note = Synthetic Construct
GAILGRSETQECLFYNANWELERTNQTGVEPCEGEKDKRLHCYATWRNISGSIEIVKKGCWLDDINCYDRTDCVETKENPQVYFCCCEGNMCNEKFSYFPEMEVTQPTS

SEQ ID NO: 33 - protein, 109 aa, synthetic construct
  REGION 1..109: note = Synthetic Construct
GAILGRSETQECLFYNANWELERTNQTGVEPCEGEKDKRLHCYATWKNISGSIEIVKKGCWLDDFNCYDRTDCVETKENPQVYFCCCEGNMCNEKFSYFPEMEVTQPTS

SEQ ID NO: 34 - protein, 109 aa, synthetic construct
  REGION 1..109: note = Synthetic Construct
GAILGRSETQECLFYNANWELERTNQTGVEPCEGEKDKRLHCYATWKNISGSIEIVKKGCWLDDINCYDRTDCVETEENPQVYFCCCEGNMCNEKFSYFPEMEVTQPTS

SEQ ID NO: 35 - protein, 109 aa, synthetic construct
  REGION 1..109: note = Synthetic Construct
GAILGRSETQECLFYNANWELERTNQTGVEPCEGEKDKRLHCFATWRNISGSIEIVKKGCWLDDFNCYDRTDCVETKENPQVYFCCCEGNMCNEKFSYFPEMEVTQPTS

SEQ ID NO: 36 - protein, 109 aa, synthetic construct
  REGION 1..109: note = Synthetic Construct
GAILGRSETQECLFYNANWELERTNQTGVEPCEGEKDKRLHCFATWRNISGSIEIVKKGCWLDDINCYDRTDCVETEENPQVYFCCCEGNMCNEKFSYFPEMEVTQPTS

SEQ ID NO: 37 - protein, 109 aa, synthetic construct
  REGION 1..109: note = Synthetic Construct
GAILGRSETQECLFYNANWELERTNQTGVEPCEGEKDKRLHCFATWKNISGSIEIVKKGCWLDDFNCYDRTDCVETEENPQVYFCCCEGNMCNEKFSYFPEMEVTQPTS

SEQ ID NO: 38 - protein, 109 aa, synthetic construct
  REGION 1..109: note = Synthetic Construct
GAILGRSETQECLFYNANWELDRTNQTGVERCEGEKDKRLHCYATWRNISGSIEIVKKGCWLDDINCYDRTDCVETKENPQVYFCCCEGNMCNEKFSYFPEMEVTQPTS

SEQ ID NO: 39 - protein, 109 aa, synthetic construct
  REGION 1..109: note = Synthetic Construct
GAILGRSETQECLFYNANWELDRTNQTGVERCEGEKDKRLHCYATWKNISGSIEIVKKGCWLDDFNCYDRTDCVETKENPQVYFCCCEGNMCNEKFSYFPEMEVTQPTS

SEQ ID NO: 40 - protein, 109 aa, synthetic construct
  REGION 1..109: note = Synthetic Construct
GAILGRSETQECLFYNANWELDRTNQTGVERCEGEKDKRLHCYATWKNISGSIEIVKKGCWLDDINCYDRTDCVETEENPQVYFCCCEGNMCNEKFSYFPEMEVTQPTS

SEQ ID NO: 41 - protein, 109 aa, synthetic construct
  REGION 1..109: note = Synthetic Construct
GAILGRSETQECLFYNANWELDRTNQTGVERCEGEKDKRLHCFATWRNISGSIEIVKKGCWLDDFNCYDRTDCVETKENPQVYFCCCEGNMCNEKFSYFPEMEVTQPTS

SEQ ID NO: 42 - protein, 109 aa, synthetic construct
  REGION 1..109: note = Synthetic Construct
GAILGRSETQECLFYNANWELDRTNQTGVERCEGEKDKRLHCFATWRNISGSIEIVKKGCWLDDINCYDRTDCVETEENPQVYFCCCEGNMCNEKFSYFPEMEVTQPTS

SEQ ID NO: 43 - protein, 109 aa, synthetic construct
  REGION 1..109: note = Synthetic Construct
GAILGRSETQECLFYNANWELDRTNQTGVERCEGEKDKRLHCFATWKNISGSIEIVKKGCWLDDFNCYDRTDCVETEENPQVYFCCCEGNMCNEKFSYFPEMEVTQPTS

SEQ ID NO: 44 - protein, 109 aa, synthetic construct
  REGION 1..109: note = Synthetic Construct
GAILGRSETQECLFYNANWELDRTNQTGVEPCEGEKDKRLHCYATWRNISGSIEIVKKGCWLDDFNCYDRTDCVETKENPQVYFCCCEGNMCNEKFSYFPEMEVTQPTS

SEQ ID NO: 45 - protein, 109 aa, synthetic construct
  REGION 1..109: note = Synthetic Construct
GAILGRSETQECLFYNANWELDRTNQTGVEPCEGEKDKRLHCYATWRNISGSIEIVKKGCWLDDINCYDRTDCVETEENPQVYFCCCEGNMCNEKFSYFPEMEVTQPTS

SEQ ID NO: 46 - protein, 109 aa, synthetic construct
  REGION 1..109: note = Synthetic Construct
GAILGRSETQECLFYNANWELDRTNQTGVEPCEGEKDKRLHCYATWKNISGSIEIVKKGCWLDDFNCYDRTDCVETEENPQVYFCCCEGNMCNEKFSYFPEMEVTQPTS

SEQ ID NO: 47 - protein, 109 aa, synthetic construct
  REGION 1..109: note = Synthetic Construct
GAILGRSETQECLFYNANWELDRTNQTGVEPCEGEKDKRLHCFATWRNISGSIEIVKKGCWLDDFNCYDRTDCVETEENPQVYFCCCEGNMCNEKFSYFPEMEVTQPTS

SEQ ID NO: 48 - protein, 109 aa, synthetic construct
  REGION 1..109: note = Synthetic Construct
GAILGRSETQECLFYNANWELERTNQTGVERCEGEKDKRLHCYATWRNISGSIEIVKKGCWLDDINCYDRTDCVETKENPQVYFCCCEGNMCNEKFSYFPEMEVTQPTS

SEQ ID NO: 49 - protein, 109 aa, synthetic construct
  REGION 1..109: note = Synthetic Construct
GAILGRSETQECLFYNANWELERTNQTGVERCEGEKDKRLHCYATWKNISGSIEIVKKGCWLDDFNCYDRTDCVETKENPQVYFCCCEGNMCNEKFSYFPEMEVTQPTS

SEQ ID NO: 50 - protein, 109 aa, synthetic construct
  REGION 1..109: note = Synthetic Construct
GAILGRSETQECLFYNANWELERTNQTGVERCEGEKDKRLHCYATWKNISGSIEIVKKGCWLDDINCYDRTDCVETEENPQVYFCCCEGNMCNEKFSYFPEMEVTQPTS

SEQ ID NO: 51 - protein, 109 aa, synthetic construct
  REGION 1..109: note = Synthetic Construct
GAILGRSETQECLFYNANWELERTNQTGVERCEGEKDKRLHCFATWRNISGSIEIVKKGCWLDDFNCYDRTDCVETKENPQVYFCCCEGNMCNEKFSYFPEMEVTQPTS

SEQ ID NO: 52 - protein, 109 aa, synthetic construct
  REGION 1..109: note = Synthetic Construct
GAILGRSETQECLFYNANWELERTNQTGVERCEGEKDKRLHCFATWRNISGSIEIVKKGCWLDDINCYDRTDCVETEENPQVYFCCCEGNMCNEKFSYFPEMEVTQPTS

SEQ ID NO: 53 - protein, 109 aa, synthetic construct
  REGION 1..109: note = Synthetic Construct
GAILGRSETQECLFYNANWELERTNQTGVERCEGEKDKRLHCFATWKNISGSIEIVKKGCWLDDFNCYDRTDCVETEENPQVYFCCCEGNMCNEKFSYFPEMEVTQPTS

SEQ ID NO: 54 - protein, 109 aa, synthetic construct
  REGION 1..109: note = Synthetic Construct
GAILGRSETQECLFYNANWELERTNQTGVEPCEGEKDKRLHCYATWRNISGSIEIVKKGCWLDDFNCYDRTDCVETKENPQVYFCCCEGNMCNEKFSYFPEMEVTQPTS

SEQ ID NO: 55 - protein, 109 aa, synthetic construct
  REGION 1..109: note = Synthetic Construct
GAILGRSETQECLFYNANWELERTNQTGVEPCEGEKDKRLHCYATWRNISGSIEIVKKGCWLDDINCYDRTDCVETEENPQVYFCCCEGNMCNEKFSYFPEMEVTQPTS

SEQ ID NO: 56 - protein, 109 aa, synthetic construct
  REGION 1..109: note = Synthetic Construct
GAILGRSETQECLFYNANWELERTNQTGVEPCEGEKDKRLHCYATWKNISGSIEIVKKGCWLDDFNCYDRTDCVETEENPQVYFCCCEGNMCNEKFSYFPEMEVTQPTS

SEQ ID NO: 57 - protein, 109 aa, synthetic construct
  REGION 1..109: note = Synthetic Construct
GAILGRSETQECLFYNANWELERTNQTGVEPCEGEKDKRLHCFATWRNISGSIEIVKKGCWLDDFNCYDRTDCVETEENPQVYFCCCEGNMCNEKFSYFPEMEVTQPTS

SEQ ID NO: 58 - protein, 109 aa, synthetic construct
  REGION 1..109: note = Synthetic Construct
GAILGRSETQECLFYNANWELDRTNQTGVERCEGEKDKRLHCYATWRNISGSIEIVKKGCWLDDFNCYDRTDCVETKENPQVYFCCCEGNMCNEKFSYFPEMEVTQPTS

SEQ ID NO: 59 - protein, 109 aa, synthetic construct
  REGION 1..109: note = Synthetic Construct
GAILGRSETQECLFYNANWELDRTNQTGVERCEGEKDKRLHCYATWRNISGSIEIVKKGCWLDDINCYDRTDCVETEENPQVYFCCCEGNMCNEKFSYFPEMEVTQPTS

SEQ ID NO: 60 - protein, 109 aa, synthetic construct
  REGION 1..109: note = Synthetic Construct
GAILGRSETQECLFYNANWELDRTNQTGVERCEGEKDKRLHCYATWKNISGSIEIVKKGCWLDDFNCYDRTDCVETEENPQVYFCCCEGNMCNEKFSYFPEMEVTQPTS

SEQ ID NO: 61 - protein, 109 aa, synthetic construct
  REGION 1..109: note = Synthetic Construct
GAILGRSETQECLFYNANWELDRTNQTGVERCEGEKDKRLHCFATWRNISGSIEIVKKGCWLDDFNCYDRTDCVETEENPQVYFCCCEGNMCNEKFSYFPEMEVTQPTS

SEQ ID NO: 62 - protein, 109 aa, synthetic construct
  REGION 1..109: note = Synthetic Construct
GAILGRSETQECLFYNANWELDRTNQTGVEPCEGEKDKRLHCYATWRNISGSIEIVKKGCWLDDFNCYDRTDCVETEENPQVYFCCCEGNMCNEKFSYFPEMEVTQPTS

SEQ ID NO: 63 - protein, 109 aa, synthetic construct
  REGION 1..109: note = Synthetic Construct
GAILGRSETQECLFYNANWELERTNQTGVERCEGEKDKRLHCYATWRNISGSIEIVKKGCWLDDFNCYDRTDCVETKENPQVYFCCCEGNMCNEKFSYFPEMEVTQPTS

SEQ ID NO: 64 - protein, 109 aa, synthetic construct
  REGION 1..109: note = Synthetic Construct
GAILGRSETQECLFYNANWELERTNQTGVERCEGEKDKRLHCYATWRNISGSIEIVKKGCWLDDINCYDRTDCVETEENPQVYFCCCEGNMCNEKFSYFPEMEVTQPTS

SEQ ID NO: 65 - protein, 109 aa, synthetic construct
  REGION 1..109: note = Synthetic Construct
GAILGRSETQECLFYNANWELERTNQTGVERCEGEKDKRLHCYATWKNISGSIEIVKKGCWLDDFNCYDRTDCVETEENPQVYFCCCEGNMCNEKFSYFPEMEVTQPTS

SEQ ID NO: 66 - protein, 109 aa, synthetic construct
  REGION 1..109: note = Synthetic Construct
GAILGRSETQECLFYNANWELERTNQTGVERCEGEKDKRLHCFATWRNISGSIEIVKKGCWLDDFNCYDRTDCVETEENPQVYFCCCEGNMCNEKFSYFPEMEVTQPTS

SEQ ID NO: 67 - protein, 109 aa, synthetic construct
  REGION 1..109: note = Synthetic Construct
GAILGRSETQECLFYNANWELDRTNQTGVERCEGEKDKRLHCYATWRNISGSIEIVKKGCWLDDFNCYDRTDCVETEENPQVYFCCCEGNMCNEKFSYFPEMEVTQPTS

SEQ ID NO: 68 - protein, 109 aa, synthetic construct
  REGION 1..109: note = Synthetic Construct
GAILGRSETQECLFYNANWELERTNQTGVEPCEGEKDKRLHCYATWRNISGSIEIVKKGCWLDDFNCYDRTDCVETEENPQVYFCCCEGNMCNEKFSYFPEMEVTQPTS

SEQ ID NO: 69 - protein, 109 aa, synthetic construct
  REGION 1..109: note = Synthetic Construct
GAILGRSETQECLFYNANWELERTNQTGVERCEGEKDKRLHCYATWRNISGSIEIVKKGCWLDDFNCYDRTDCVETEENPQVYFCCCEGNMCNEKFSYFPEMEVTQPTS

SEQ ID NO: 70 - protein, 109 aa, synthetic construct
  REGION 1..109: note = Synthetic Construct
GAILGRSETQECLYYNANWELERTNQTGVERCEGEQDKRLHCYATWRNISGSIEIVKKGCWLDDFNCYDRTDCVETEENPQVYFCCCEGNMCNEKFSYFPEMEVTQPTS

SEQ ID NO: 71 - protein, 111 aa, synthetic construct
  REGION 1..111: note = Synthetic Construct
GAILGRSETQECLFYNANWELERTNQTGVERCEGEKDKRLHCYATWRNISGSIEIVKKGCWLDDFNCYDRTDCVETEENPQVYFCCCEGNMCNEKFSYFPEMEVTQPTSNP

SEQ ID NO: 72 - protein, 115 aa, synthetic construct
  REGION 1..115: note = Synthetic Construct
GAILGRSETQECLFYNANWELERTNQTGVERCEGEKDKRLHCYATWRNISGSIEIVKKGCWLDDFNCYDRTDCVETEENPQVYFCCCEGNMCNEKFSYFPEMEVTQPTSNPVTPK

SEQ ID NO: 73 - protein, 109 aa, Homo sapiens
GAILGRSETQECLFFNANWEKDRTNQTGVEPCYGDKDKRRHCFATWKNISGSIEIVKQGCWLDDINCYDRTDCVEKKDSPEVYFCCCEGNMCNEKFSYFPEMEVTQPTS

SEQ ID NO: 74 - protein, 115 aa, Homo sapiens
GRGEAETRECIYYNANWELERTNQSGLERCEGEQDKRLHCYASWRNSSGTIELVKKGCWLDDFNCYDRQECVATEENPQVYFCCCEGNFCNERFTHLPEAGGPEVTYEPPPTAPT

SEQ ID NO: 75 - protein, 513 aa, Homo sapiens
MGAAAKLAFAVFLISCSSGAILGRSETQECLFFNANWEKDRTNQTGVEPCYGDKDKRRHCFATWKNISGSIEIVKQGCWLDDINCYDRTDCVEKKDSPEVYFCCCEGNMCNEKFSYFPEMEVTQPTSNPVTPKPPYYNILLYSLVPLMLIAGIVICAFWVYRHHKMAYPPVLVPTQDPGPPPPSPLLGLKPLQLLEVKARGRFGCVWKAQLLNEYVAVKIFPIQDKQSWQNEYEVYSLPGMKHENILQFIGAEKRGTSVDVDLWLITAFHEKGSLSDFLKANVVSWNELCHIAETMARGLAYLHEDIPGLKDGHKPAISHRDIKSKNVLLKNNLTACIADFGLALKFEAGKSAGDTHGQVGTRRYMAPEVLEGAINFQRDAFLRIDMYAMGLVLWELASRCTAADGPVDEYMLPFEEEIGQHPSLEDMQEVVVHKKKRPVLRDYWQKHAGMAMLCETIEECWDHDAEARLSAGCVGERITQMQRLTNIITTEDIVTVVTMVTNVDFPPKESSL

SEQ ID NO: 76 - protein, 4 aa, Homo sapiens
TEEN

SEQ ID NO: 77 - protein, 4 aa, synthetic construct
  REGION 1..4: note = Synthetic Construct
TKEN

SEQ ID NO: 78 - protein, 6 aa, Homo sapiens
NPVTPK

SEQ ID NO: 79 - protein, 226 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 80 - protein, 338 aa, synthetic construct
  REGION 1..338: note = Synthetic Construct
GAILGRSETQECLFYNANWELERTNQTGVERCEGEKDKRLHCYATWRNISGSIEIVKKGCWLDDFNCYDRTDCVETEENPQVYFCCCEGNMCNEKFSYFPEMEVTQPTSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 81 - protein, 609 aa, Homo sapiens [sequence omitted]

SEQ ID NO: 82 - protein, 93 aa, Homo sapiens
GPVEVFITETPSQPNSHPIQWNAPQPSHISKYILRWRPKNSVGRWKEATIPGHLNSYTIKGLKPGVVYEGQLISIQQYGHQEVTRFDFTTTST

SEQ ID NO: 83 - protein, 11 aa, synthetic construct
  REGION 1..11: note = Synthetic Construct
DICLPRWGCLW

SEQ ID NO: 84 - protein, 227 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 85 - protein, 109 aa, synthetic construct
  REGION 1..109: note = Synthetic Construct
GAILGRSETQECLFYNANWELERTNQTGVERCEGEKDKRLHCYATWRNISGSIEIVAKGCWLDDFNCYDRTDCVETEENPQVYFCCCEGNMCNEKFSYFPEMEVTQPTS

SEQ ID NO: 86 - protein, 5 aa, synthetic construct
  REGION 1..5: note = Synthetic Construct
PAPAP

SEQ ID NO: 87 - protein, 5 aa, synthetic construct
  REGION 1..5: note = Synthetic Construct
EAAAK

SEQ ID NO: 88 - protein, 36 aa, synthetic construct
  REGION 1..36: note = Synthetic Construct
GGSGGGSEGGGSEGGGSEGGGSEGGGSEGGGSGGGS

SEQ ID NO: 89 - protein, 17 aa, synthetic construct
  REGION 1..17: note = Synthetic Construct
AAANSSIDLISVPVDSR

SEQ ID NO: 90 - protein, 36 aa, synthetic construct
  REGION 1..36: note = Synthetic Construct
GGSAGGSGSGSSGGSSGASGTGTAGGTGSGSGTGSG

SEQ ID NO: 91 - protein, 17 aa, synthetic construct
  REGION 1..17: note = Synthetic Construct
RPACKIPNDLKQKVMNH

SEQ ID NO: 92 - protein, 5 aa, synthetic construct
  REGION 1..5: note = Synthetic Construct
RSIAT

SEQ ID NO: 93 - protein, 8 aa, synthetic construct
  REGION 1..8: note = Synthetic Construct
SACYCELS

SEQ ID NO: 94 - protein, 10 aa, synthetic construct
  REGION 1..10: note = Synthetic Construct
GENLYFQSGG

SEQ ID NO: 95 - protein, 18 aa, synthetic construct
  REGION 1..18: note = Synthetic Construct
KESGSVSSEQLAQFRSLD

SEQ ID NO: 96 - protein, 12 aa, synthetic construct
  REGION 1..12: note = Synthetic Construct
AEAAAKEAAAKA

SEQ ID NO: 97 - protein, 225 aa, Homo sapiens
THTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPVPIEKTISKAKGQPREPQVYTLPPSREEMTKNQVSLTCLVKGFYPSDIAVEWESNGQPENNYKTTPPVLDSDGPFFLYSKLTVDKSRWQQGNVFSCSVMHEALHNHYTQKSLSLSPGK

SEQ ID NO: 98 - protein, 4 aa, synthetic construct
  REGION 1..4: note = Synthetic Construct
GGGA

SEQ ID NO: 99 - protein, 4 aa, synthetic construct
  REGION 1..4: note = Synthetic Construct
GGGS

SEQ ID NO: 100 - protein, 4 aa, synthetic construct
  REGION 1..4: note = Synthetic Construct
GGGG

SEQ ID NO: 101 - protein, 5 aa, synthetic construct
  REGION 1..5: note = Synthetic Construct
GGGGA

SEQ ID NO: 102 - protein, 5 aa, synthetic construct
  REGION 1..5: note = Synthetic Construct
GGGGS

SEQ ID NO: 103 - protein, 5 aa, synthetic construct
  REGION 1..5: note = Synthetic Construct
GGGGG

SEQ ID NO: 104 - protein, 4 aa, synthetic construct
  REGION 1..4: note = Synthetic Construct
GGAG

SEQ ID NO: 105 - protein, 4 aa, synthetic construct
  REGION 1..4: note = Synthetic Construct
GGSG

SEQ ID NO: 106 - protein, 4 aa, synthetic construct
  REGION 1..4: note = Synthetic Construct
AGGG

SEQ ID NO: 107 - protein, 4 aa, synthetic construct
  REGION 1..4: note = Synthetic Construct
SGGG

SEQ ID NO: 108 - protein, 4 aa, synthetic construct
  REGION 1..4: note = Synthetic Construct
GAGA

SEQ ID NO: 109 - protein, 4 aa, synthetic construct
  REGION 1..4: note = Synthetic Construct
GSGS

SEQ ID NO: 110 - protein, 6 aa, synthetic construct
  REGION 1..6: note = Synthetic Construct
GAGAGA

SEQ ID NO: 111 - protein, 6 aa, synthetic construct
  REGION 1..6: note = Synthetic Construct
GSGSGS

SEQ ID NO: 112 - protein, 8 aa, synthetic construct
  REGION 1..8: note = Synthetic Construct
GAGAGAGA

SEQ ID NO: 113 - protein, 8 aa, synthetic construct
  REGION 1..8: note = Synthetic Construct
GSGSGSGS

SEQ ID NO: 114 - protein, 10 aa, synthetic construct
  REGION 1..10: note = Synthetic Construct
GAGAGAGAGA

SEQ ID NO: 115 - protein, 10 aa, synthetic construct
  REGION 1..10: note = Synthetic Construct
GSGSGSGSGS

SEQ ID NO: 116 - protein, 12 aa, synthetic construct
  REGION 1..12: note = Synthetic Construct
GAGAGAGAGAGA

SEQ ID NO: 117 - protein, 12 aa, synthetic construct
  REGION 1..12: note = Synthetic Construct
GSGSGSGSGSGS

SEQ ID NO: 118 - protein, 6 aa, synthetic construct
  REGION 1..6: note = Synthetic Construct
GGAGGA

SEQ ID NO: 119 - protein, 6 aa, synthetic construct
  REGION 1..6: note = Synthetic Construct
GGSGGS

SEQ ID NO: 120 - protein, 9 aa, synthetic construct
  REGION 1..9: note = Synthetic Construct
GGAGGAGGA

SEQ ID NO: 121 - protein, 9 aa, synthetic construct
  REGION 1..9: note = Synthetic Construct
GGSGGSGGS

SEQ ID NO: 122 - protein, 12 aa, synthetic construct
  REGION 1..12: note = Synthetic Construct
GGAGGAGGAGGA

SEQ ID NO: 123 - protein, 12 aa, synthetic construct
  REGION 1..12: note = Synthetic Construct
GGSGGSGGSGGS

SEQ ID NO: 124 - protein, 8 aa, synthetic construct
  REGION 1..8: note = Synthetic Construct
GGAGGGAG

SEQ ID NO: 125 - protein, 8 aa, synthetic construct
  REGION 1..8: note = Synthetic Construct
GGSGGGSG

SEQ ID NO: 126 - protein, 12 aa, synthetic construct
  REGION 1..12: note = Synthetic Construct
GGAGGGAGGGAG

SEQ ID NO: 127 - protein, 12 aa, synthetic construct
  REGION 1..12: note = Synthetic Construct
GGSGGGSGGGSG

SEQ ID NO: 128 - protein, 15 aa, synthetic construct
  REGION 1..15: note = Synthetic Construct
GGGGAGGGGAGGGGA

SEQ ID NO: 129 - protein, 15 aa, synthetic construct
  REGION 1..15: note = Synthetic Construct
GGGGSGGGGSGGGGS

SEQ ID NO: 130 - protein, 5 aa, synthetic construct
  REGION 1..5: note = Synthetic Construct
GGGAG

SEQ ID NO: 131 - protein, 6 aa, synthetic construct
  REGION 1..6: note = Synthetic Construct
GGGAGG

SEQ ID NO: 132 - protein, 7 aa, synthetic construct
  REGION 1..7: note = Synthetic Construct
GGGAGGG

SEQ ID NO: 133 - protein, 4 aa, synthetic construct
  REGION 1..4: note = Synthetic Construct
AAAL

SEQ ID NO: 134 - protein, 4 aa, synthetic construct
  REGION 1..4: note = Synthetic Construct
AAAK

SEQ ID NO: 135 - protein, 4 aa, synthetic construct
  REGION 1..4: note = Synthetic Construct
AAAR

SEQ ID NO: 136 - protein, 14 aa, synthetic construct
  REGION 1..14: note = Synthetic Construct
EGKSSGSGSESKST

SEQ ID NO: 137 - protein, 12 aa, synthetic construct
  REGION 1..12: note = Synthetic Construct
GSAGSAAGSGEF

SEQ ID NO: 138 - protein, 126 aa, synthetic construct
  REGION 1..126: note = Synthetic Construct
QVQLQESGPGLVKPSETLSLTCTVSGGSFSSHFWSWIRQPPGKGLEWIGYILYTGGTSFNPSLKSRVSMSVGTSKNQFSLKLSSVTAADTAVYYCARARSGITFTGIIVPGSFDIWGQGTMVTVSS

SEQ ID NO: 139 - protein, 108 aa, synthetic construct
  REGION 1..108: note = Synthetic Construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPWTFGQGTKVEIK

SEQ ID NO: 140 - protein, 122 aa, synthetic construct
  REGION 1..122: note = Synthetic Construct
QVQLQQSGPGLLKPSQTLSLTCAISGDSVSSNSAAWSWIRQSPSRGLEWLGRTYYRANWFNDYALSVKSRITINPVTSTNHFSLQLHSVTPEDTAVYYCAREGALGYYFDSWGQGTLVTVSS

SEQ ID NO: 141 - protein, 113 aa, synthetic construct
  REGION 1..113: note = Synthetic Construct
DIVMTQSPDSLAVSLGERATINCKSSQSVLYSSNNKNYLAWYQQKPGQPPTLLIYWASTRESGVPDRFSGSGSGTDFTLTISSLQAEDVAIYYCHQYFITPLTFGGGTKVEIK

SEQ ID NO: 142 - protein, 126 aa, synthetic construct
  REGION 1..126: note = Synthetic Construct
QVQLQESGPGLVKPSETLSITCTVSGGSFSSHFWTWIRQPPGKGLEWIGYLHYSGGTSYNPSLKSRVIISVDTSKNQFSLKLNSVTAADTAVYYCARARSGITFGGLIVPGSFDIWGQGTMVTVSS

SEQ ID NO: 143 - protein, 126 aa, synthetic construct
  REGION 1..126: note = Synthetic Construct
QVQLQESGPGLVKPSETLSLTCTVSGGSFSSHFWSWIRQPPGKGLEWIGYIYYSGGTHYNPSLESRVTISVDTSKNQFSLKLNSVTAADTAVYYCARARSGITFGGLIVPGSFDIWGQGTMVTVSS

SEQ ID NO: 144 - protein, 125 aa, synthetic construct
  REGION 1..125: note = Synthetic Construct
EVQLVESGGGLVQPGRSLRLSCKASGFAFDDFAMHWVRQAPGKGLEWVSGIVWNSGDIGYADSVKGRFTISRDNAKNSLYLQLNSLRTEDTALYFCVKDMVRGLMGFNYYGMDVWGQGTTVTVSS

SEQ ID NO: 145 - protein, 107 aa, synthetic construct
  REGION 1..107: note = Synthetic Construct
EIVLTQSPATLSLSPGERATLSCRASQTISTYLVWYRQRPGQAPSLLIYDASNRATDIPARFSGSGSGTDFTLTISSLEPEDFAVYYCQQRSNWPITFGQGTRLEIK

SEQ ID NO: 146 - protein, 117 aa, synthetic construct
  REGION 1..117: note = Synthetic Construct
QVQLVESGGGVVQPGRSLRLSCVASGFTVSSYGIHWVRQAPGKGLEWVSVIWYDGRNKDYADSVKGRFTISRDNSKNTVYLEMKGLRAEDTALYYCARDKTGDFDSWGQGTLVTVSS

SEQ ID NO: 147 - protein, 107 aa, synthetic construct
  REGION 1..107: note = Synthetic Construct
DIQMTQSPSSLSASVGDRVTITCRASQNINSFLSWYQQKPGKAPKFLIYDASSIQSGAPSRFSGSGSGTDFTLTISSLQPEDFATYYCQQSYSSPFTFGQGTKLEIK

SEQ ID NO: 148 - protein, 123 aa, synthetic construct
  REGION 1..123: note = Synthetic Construct
EVQLVESGGGLVQPGRSLTLSCAVSGFTFDDHAMHWVRQAPGKGLEWVSGISWNSVSIGYADSVKGRFTISRDNAKTSLYLQMNSLRVDDTALYYCVQDLNDILTGYPLFDFWGQGTLVTVSS

SEQ ID NO: 149 - protein, 108 aa, synthetic construct
  REGION 1..108: note = Synthetic Construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIK

SEQ ID NO: 150 - protein, 117 aa, synthetic construct
  REGION 1..117: note = Synthetic Construct
QVQLVESGGGVVQPGRSLRLTCVASGFTVSSYGMHWVRQAPGKGLEWVAVMFYDESKKYYADSVKGRFTISRDNSKNTLYLQMNSLRVEDTAVYYCARDEQLDFEYWGQGTLVTVSS

SEQ ID NO: 151 - protein, 126 aa, synthetic construct
  REGION 1..126: note = Synthetic Construct
EVQLVESGGGVVQPGGSLRLSCAASTFTFDDFAMHWVRQAPGKGLEWVSLITGDGGSTYYADPVKGRFTISRDNSKNSLYLQMNSLRTEDTALYYCVKDWWIAARPDYYYYGMDVWGQGTTVTVSS

SEQ ID NO: 152 - protein, 8 aa, synthetic construct
  REGION 1..8: note = Synthetic Construct
GGSFSSHF

SEQ ID NO: 153 - protein, 7 aa, synthetic construct
  REGION 1..7: note = Synthetic Construct
ILYTGGT

SEQ ID NO: 154 - protein, 20 aa, synthetic construct
  REGION 1..20: note = Synthetic Construct
ARARSGITFTGIIVPGSFDI

SEQ ID NO: 155 - protein, 7 aa, synthetic construct
  REGION 1..7: note = Synthetic Construct
QSVSSSY

SEQ ID NO: 156 - skipped

SEQ ID NO: 157 - protein, 9 aa, synthetic construct
  REGION 1..9: note = Synthetic Construct
QQYGSSPWT

SEQ ID NO: 158 - protein, 8 aa, synthetic construct
  REGION 1..8: note = Synthetic Construct
GFAFDDFA

SEQ ID NO: 159 - protein, 8 aa, synthetic construct
  REGION 1..8: note = Synthetic Construct
IVWNSGDI

SEQ ID NO: 160 - protein, 18 aa, synthetic construct
  REGION 1..18: note = Synthetic Construct
VKDMVRGLMGFNYYGMDV

SEQ ID NO: 161 - protein, 6 aa, synthetic construct
  REGION 1..6: note = Synthetic Construct
QTISTY

SEQ ID NO: 162 - skipped

SEQ ID NO: 163 - protein, 9 aa, synthetic construct
  REGION 1..9: note = Synthetic Construct
QQRSNWPIT

SEQ ID NO: 164 - protein, 120 aa, synthetic construct
  REGION 1..120: note = Synthetic Construct
EVQVLESGGDLVQPGGSLRLSCAASGFTFSAYAMTWVRQAPGKGLEWVSAISGSGGSAYYADSVKGRFTISRDNSKNTVYLQMNSLRAEDTAVYYCAKDGAWKMSGLDVWGQGTTVIVSS

SEQ ID NO: 165 - protein, 107 aa, synthetic construct
  REGION 1..107: note = Synthetic Construct
DIQMTQSPASLSASVGDRVTITCRASQDISDYLAWYQQKPGKIPRLLIYTTSTLQSGVPSRFRGSGSGTDFTLTISSLQPEDVATYYCQKYDSAPLTFGGGTKVEIK

SEQ ID NO: 166 - protein, 8 aa, synthetic construct
  REGION 1..8: note = Synthetic Construct
GFTFSAYA

SEQ ID NO: 167 - protein, 8 aa, synthetic construct
  REGION 1..8: note = Synthetic Construct
ISGSGGSA

SEQ ID NO: 168 - protein, 13 aa, synthetic construct
  REGION 1..13: note = Synthetic Construct
AKDGAWKMSGLDV

SEQ ID NO: 169 - protein, 6 aa, synthetic construct
  REGION 1..6: note = Synthetic Construct
QDISDY

SEQ ID NO: 170 - skipped

SEQ ID NO: 171 - protein, 9 aa, synthetic construct
  REGION 1..9: note = Synthetic Construct
QKYDSAPLT

SEQ ID NO: 172 - protein, 122 aa, synthetic construct
  REGION 1..122: note = Synthetic Construct
QVQLVQSGAEVKKPGASVKVSCKASGYTFTSYGLSWVRQAPGQGLEWMGWIIPYNGNTNSAQKLQGRVTMTTDTSTSTAYMELRSLRSDDTAVYFCARDRDYGVNYDAFDIWGQGTMVTVSS

SEQ ID NO: 173 - protein, 106 aa, synthetic construct
  REGION 1..106: note = Synthetic Construct
SYEVTQAPSVSVSPGQTASITCSGDKLGDKYACWYQQKPGQSPVLVIYQDSKRPSGIPERFSGSNSGNTATLTISGTQAMDEADYYCQAWDSSTAVFGGGTKLTVL

SEQ ID NO: 174 - protein, 123 aa, synthetic construct
  REGION 1..123: note = Synthetic Construct
EVQLVESGGGLVQPGGSLRLSCAASGFTFSSYWMSWVRQAPGKGLECVANIKQDGSEEYYVDSVKGRFTISRDNAKNSLYLQMNSLRAEDTAVYYCARGSSSWYYYNYGMDVWGQGTTVTVSS

SEQ ID NO: 175 - protein, 107 aa, synthetic construct
  REGION 1..107: note = Synthetic Construct
DIQMTQSPSSLSASVGDRVTITCRASQGIRNDLGWYQQKPGKAPKRLIYAASSLQSGVPSRFSGSGSGTEFTLTISSLQPEDFATYYCRQQNTYPLTFGGGTKVEIK

SEQ ID NO: 176 - protein, 10 aa, synthetic construct
  REGION 1..10: note = Synthetic Construct
GYTFTSYGLS

SEQ ID NO: 177 - protein, 17 aa, synthetic construct
  REGION 1..17: note = Synthetic Construct
WIIPYNGNTNSAQKLQG

SEQ ID NO: 178 - protein, 13 aa, synthetic construct
  REGION 1..13: note = Synthetic Construct
DRDYGVNYDAFDI

SEQ ID NO: 179 - protein, 11 aa, synthetic construct
  REGION 1..11: note = Synthetic Construct
SGDKLGDKYAC

SEQ ID NO: 180 - protein, 7 aa, synthetic construct
  REGION 1..7: note = Synthetic Construct
QDSKRPS

SEQ ID NO: 181 - protein, 9 aa, synthetic construct
  REGION 1..9: note = Synthetic Construct
QAWDSSTAV

SEQ ID NO: 182 - protein, 10 aa, synthetic construct
  REGION 1..10: note = Synthetic Construct
GFTFSSYWMS

SEQ ID NO: 183 - protein, 17 aa, synthetic construct
  REGION 1..17: note = Synthetic Construct
NIKQDGSEEYYVDSVKG

SEQ ID NO: 184 - protein, 14 aa, synthetic construct
  REGION 1..14: note = Synthetic Construct
GSSSWYYYNYGMDV

SEQ ID NO: 185 - protein, 11 aa, synthetic construct
  REGION 1..11: note = Synthetic Construct
RASQGIRNDLG

SEQ ID NO: 186 - protein, 7 aa, synthetic construct
  REGION 1..7: note = Synthetic Construct
AASSLQS

SEQ ID NO: 187 - protein, 9 aa, synthetic construct
  REGION 1..9: note = Synthetic Construct
RQQNTYPLT

SEQ ID NO: 188 - protein, 113 aa, synthetic construct
  REGION 1..113: note = Synthetic Construct
EVQLVESGGGLVQPGGSLRLSCAASGLTFSRYPMSWVRQAPGKGLVWVSAITSSGGSTYYSDTVKGRFTISRDNAKNTLYLQMNSLRAEDTAVYYCARLPDYWGQGTLVTVSS

SEQ ID NO: 189 - protein, 108 aa, synthetic construct
  REGION 1..108: note = Synthetic Construct
EIVLTQSPGTLSLSPGERATLSCRASSSVSSSYLHWYQQKPGQAPRLLIYSTSNLVAGIPDRFSGSGSGTDFTLTISRLEPEDFAVYYCQHHSGYHFTFGGGTKVEIK

SEQ ID NO: 190 - protein, 10 aa, synthetic construct
  REGION 1..10: note = Synthetic Construct
  VARIANT 3: note = Xaa is Thr or Asn
  VARIANT 8: note = Xaa is Gly, Thr or Pro
GLXFSRYXMS

SEQ ID NO: 191 - protein, 17 aa, synthetic construct
  REGION 1..17: note = Synthetic Construct
  VARIANT 1: note = Xaa is Ala, Arg or His
  VARIANT 2: note = Xaa is IIe or Gln
  VARIANT 3: note = Xaa is Asn, Thr or Lys
  VARIANT 4: note = Xaa is Ser or Ala
  VARIANT 5: note = Xaa is His, Ser, Val, Leu, Met, Asn, Gly, Asp, Trp, Arg, Ala,Tyr, Lys, Pro, Thr, IIe or Glu
  VARIANT 7: note = Xaa is Gly or Ser
  VARIANT 10: note = Xaa is Tyr or Lys
XXXXXGXSTXYSDTVKG

SEQ ID NO: 192 - protein, 4 aa, synthetic construct
  REGION 1..4: note = Synthetic Construct
LPDY

SEQ ID NO: 193 - protein, 12 aa, synthetic construct
  REGION 1..12: note = Synthetic Construct
  VARIANT 3: note = Xaa is Ser or Leu
  VARIANT 4: note = Xaa is Ser or Gln
RAXXSVSSSYLH

SEQ ID NO: 194 - protein, 7 aa, synthetic construct
  REGION 1..7: note = Synthetic Construct
  VARIANT 6: note = Xaa is Ala, Val, Thr, or Met
  VARIANT 7: note = Xaa is Ser, Ala, Thr, Asn, Trp, Asp or Tyr
STSNLXX

SEQ ID NO: 195 - protein, 9 aa, synthetic construct
  REGION 1..9: note = Synthetic Construct
  VARIANT 2: note = Xaa is Gln, Asn, His, Leu, Pro or Trp
  VARIANT 3: note = Xaa is Tyr or His
  VARIANT 4: note = Xaa is Ser or Leu
  VARIANT 7: note = Xaa is His, Phe, Gln or Thr
  VARIANT 8: note = Xaa is Phe or Trp
QXXXGYXXT

SEQ ID NO: 196 - protein, 10 aa, synthetic construct
  REGION 1..10: note = Synthetic Construct
GLTFSRYPMS

SEQ ID NO: 197 - protein, 17 aa, synthetic construct
  REGION 1..17: note = Synthetic Construct
AITSSGGSTYYSDTVKG

SEQ ID NO: 198 - protein, 12 aa, synthetic construct
  REGION 1..12: note = Synthetic Construct
RASSSVSSSYLH

SEQ ID NO: 199 - protein, 7 aa, synthetic construct
  REGION 1..7: note = Synthetic Construct
STSNLVA

SEQ ID NO: 200 - protein, 9 aa, synthetic construct
  REGION 1..9: note = Synthetic Construct
QHHSGYHFT

SEQ ID NO: 201 - protein, 124 aa, synthetic construct
  REGION 1..124: note = Synthetic Construct
QVTLRESGPALVKPTQTLTLTCTFSGFSLRKVGSSVSWIRQPPGKALEWLAHIYWDDDKRYNPSLRNRLTISKDTSKNQVVLTMTNMDPVDTATYYCARRAITTVIGGGTFDYWGQGTTVTVSS

SEQ ID NO: 202 - protein, 106 aa, synthetic construct
  REGION 1..106: note = Synthetic Construct
DIQMTQSPSSLSASVGDRVTITCSASSSISYMHWYQQKPGKAPKLLIYDTSKLAVGVPSRFSGSGSGTDFTLTISSLQPEDFATYYCQQWYRNPLTFGGGTKVEIK

SEQ ID NO: 203 - protein, 106 aa, synthetic construct
  REGION 1..106: note = Synthetic Construct
DIQMTQSPSSLSASVGDRVTITCSASSSISYMHWYQQKPGKAPKLLIYDTSKLARGVPSRFSGSGSGTDFTLTISSLQPEDFATYYCQQWYRNPLTFGGGTKVEIK

SEQ ID NO: 204 - protein, 124 aa, synthetic construct
  REGION 1..124: note = Synthetic Construct
QVTLRESGPALVKPTQTLTLTCTFSGFSLRKVGSSVSWIRQPPGKALEWLAHIYWDDDKRYNPSLRNRLTISKDTSKNQVVLTMTNMDPVDTATYYCARRAITTVIGGGTMDYWGQGTTVTVSS

SEQ ID NO: 205 - protein, 123 aa, synthetic construct
  REGION 1..123: note = Synthetic Construct
QVTLRESGPALVKPTQTLTLTCTFSGFSLRKVGRSVSWIRQPPGKALEWLAHIYWDDDKRYNPSLRNRLTISKDTSKNQVVLTMTNMDPVDTATYYCARRAITTVIGGGTFDYWGQGTTVVSS

SEQ ID NO: 206 - protein, 124 aa, synthetic construct
  REGION 1..124: note = Synthetic Construct
QVTLRESGPALVKPTQTLTLTCTFSGFSWRKVGSSVSWIRQPPGKALEWLAHIYWDDDKRYNPSLRNRLTISKDTSKNQVVLTMTNMDPVDTATYYCARRAITTVIGGGTFDYWGQGTTVTVSS

SEQ ID NO: 207 - protein, 124 aa, synthetic construct
  REGION 1..124: note = Synthetic Construct
QVTLRESGPALVKPTQTLTLTCTFSGFSLRKVGSSVSWIRQPPGKALEWLAHIYWDDDLRYNPSLRNRLTISKDTSKNQVVLTMTNMDPVDTATYYCARRAITTVIGGGTFDYWGQGTTVTVSS

SEQ ID NO: 208 - protein, 124 aa, synthetic construct
  REGION 1..124: note = Synthetic Construct
QVTLRESGPALVKPTQTLTLTCTFSGFSMRKVGSSVSWIRQPPGKALEWLAHIYWDDDKRYNPSLRNRLTISKDTSKNQVVLTMTNMDPVDTATYYCARRAITTVIGGGTFDYWGQGTTVTVSS

SEQ ID NO: 209 - protein, 124 aa, synthetic construct
  REGION 1..124: note = Synthetic Construct
QVTLRESGPALVKPTQTLTLTCTFSGFSLRKVGSSISWIRQPPGKALEWLAHIYWDDDKRYNPSLRNRLTISKDTSKNQVVLTMTNMDPVDTATYYCARRAITTVIGGGTFDYWGQGTTVTVSS

SEQ ID NO: 210 - protein, 124 aa, synthetic construct
  REGION 1..124: note = Synthetic Construct
QVTLRESGPALVKPTQTLTLTCTFSGFSLRKVGSSVSWIRQPPGKALEWLAHIYWDDDKRYNPSLRNRLTISKDTSKNQVVLTMTNMDPVDTATYYCARRKITTVIGGGTFDYWGQGTTVTVSS

SEQ ID NO: 211-220 - skipped (10 entries)

SEQ ID NO: 221 - protein, 106 aa, synthetic construct
  REGION 1..106: note = Synthetic Construct
DIQMTQSPSSLSASVGDRVTITCSASSSISYMHWYQQKPGKAPKLLIYDTSKLARGVPSRFSGSGSGTDFTLTISSLQPEDFATYYCQQWYSNPLTFGGGTKVEIK

SEQ ID NO: 222 - protein, 124 aa, synthetic construct
  REGION 1..124: note = Synthetic Construct
QLQLQESGPGLVKPSETLSLTCTVSGFSLRRVGSSVSWIRQPPGKGLEWIGHIYWDDDKRYNPSLRNRVTISVDTSKNQFSLKLSSVTAADTAVYYCARRAITTVIGGGTFDYWGQGTLVTVSS

SEQ ID NO: 223 - protein, 124 aa, synthetic construct
  REGION 1..124: note = Synthetic Construct
QLQLQESGPGLVKPSETLSLTCTVSGFSLRMVGSSVSWIRQPPGKGLEWIGHIYWDDDKRLNPSLRNRVTISVDTSKNQFSLKLSSVTAADTAVYYCARRAITTVIGGGTFDYWGQGTLVTVSS

SEQ ID NO: 224 - protein, 124 aa, synthetic construct
  REGION 1..124: note = Synthetic Construct
QLQLQESGPGLVKPSETLSLTCTVSGFSLRKVGSSVSWIRQPPGKGLEWIGHIYWDDDLRYNPSLRNRVTISVDTSKNQFSLKLSSVTAADTAVYYCARRAITTVIGGGTFDYWGQGTLVTVSS

SEQ ID NO: 225 - protein, 124 aa, synthetic construct
  REGION 1..124: note = Synthetic Construct
QLQLQESGPGLVKPSETLSLTCTVSGFSLRKVGSSVSWIRQPPGKGLEWIGHIYWDDDKRYNPSLRNRVTISVDTSKNQFSLKLSSVTAADTAVYYCARRAITTVIGGGTFDMWGQGTLVTVSS

SEQ ID NO: 226 - protein, 124 aa, synthetic construct
  REGION 1..124: note = Synthetic Construct
QLQLQESGPGLVKPSETLSLTCTVSGFSLRKVGSSVSWIRQPPGKGLEWIGHIYWDDDKRYNPSLRNRVTISVDTSKNQFSLKLSSVTAADTAVYYCARRAITTVIGGGTFDLWGQGTLVTVSS

SEQ ID NO: 227 - protein, 124 aa, synthetic construct
  REGION 1..124: note = Synthetic Construct
QLQLQESGPGLVKPSETLSLTCTVSGFSLRKVGSSVSWIRQPPGKGLEWIGHIYWDDDKRLNPSLRNRVTISVDTSKNQFSLKLSSVTAADTAVYYCARRAITTVIGGGTFDYWGQGTLVTVSS

SEQ ID NO: 228 - protein, 124 aa, synthetic construct
  REGION 1..124: note = Synthetic Construct
QLQLQESGPGLVKPSETLSLTCTVSGFSLRKVGSSVSWIRQPPGKGLEWIGHIYWDDDKRYNPSLRNRVTISVDTSKNQFSLKLSSVTAADTAVYYCARRAITTVIGGGTFDYWGQGTLVTVSS

SEQ ID NO: 229 - protein, 106 aa, synthetic construct
  REGION 1..106: note = Synthetic Construct
DIQMTQSPSSLSASVGDRVTITCSASSSISYSHWYQQKPGKAPKLLIYDTSKLARGVPSRFSGSGSGTDFTLTISSLQPEDFATYYCQQWYSNPLTFGGGTKVEIK

SEQ ID NO: 230 - protein, 106 aa, synthetic construct
  REGION 1..106: note = Synthetic Construct
DIQMTQSPSSLSASVGDRVTITCSASSSISYAHWYQQKPGKAPKLLIYDTSKLARGVPSRFSGSGSGTDFTLTISSLQPEDFATYYCQQWYSNPLTFGGGTKVEIK

SEQ ID NO: 231 - protein, 106 aa, synthetic construct
  REGION 1..106: note = Synthetic Construct
DIQMTQSPSSLSASVGDRVTITCSASSSISYMHWYQQKPGKAPKLLIYDTSKLARGVPSRFSGSGSGTDFTLTISSLQPEDFATYYCQQWYLNPLTFGGGTKVEIK

SEQ ID NO: 232 - protein, 106 aa, synthetic construct
  REGION 1..106: note = Synthetic Construct
DIQMTQSPSSLSASVGDRVTITCSASSSISYMHWYQQKPGKAPKLLIYDTSKLARGVPSRFSGSGSGTDFTLTISSLQPEDFATYYCQQWYENPLTFGGGTKVEIK

SEQ ID NO: 233 - protein, 106 aa, synthetic construct
  REGION 1..106: note = Synthetic Construct
DIQMTQSPSSLSASVGDRVTITCSASSSISYMHWYQQKPGKAPKLLIYDTSKLARGVPSRFSGSGSGTDFTLTISSLQPEDFATYYCQQWYFNPLTFGGGTKVEIK

SEQ ID NO: 234 - protein, 124 aa, synthetic construct
  REGION 1..124: note = Synthetic Construct
QLQLQESGPGLVKPSETLSLTCTVSGFSLRKVGSSVSWIRQPPGKGLEWIGHIYWDDDKRLNPSLRNRVTISVDTSKNQFSLKLSSVTAADTAVYYCARRAITTVIGGGTFDLWGQGTLVTVSS

SEQ ID NO: 235 - protein, 12 aa, synthetic construct
  REGION 1..12: note = Synthetic Construct
  VARIANT 4: note = Xaa is Leu, Trp, or Met
  VARIANT 9: note = Xaa is Ser or Arg
  VARIANT 6: note = Xaa is Lys, Arg, or Met
GFSXRXVGXSVS

SEQ ID NO: 236 - protein, 16 aa, synthetic construct
  REGION 1..16: note = Synthetic Construct
  VARIANT 8: note = Xaa is Lys or Leu
  VARIANT 10: note = Xaa is Tyr or Leu
HIYWDDDXRXNPSLRN

SEQ ID NO: 237 - protein, 14 aa, synthetic construct
  REGION 1..14: note = Synthetic Construct
  VARIANT 2: note = Xaa is Ala or Lys
  VARIANT 12: note = Xaa is Met or Phe
  VARIANT 14: note = Xaa is Met or Leu
RXITTVIGGGTXDX

SEQ ID NO: 238 - protein, 10 aa, synthetic construct
  REGION 1..10: note = Synthetic Construct
SASSSISYMH

SEQ ID NO: 239 - protein, 7 aa, synthetic construct
  REGION 1..7: note = Synthetic Construct
DTSKLAR

SEQ ID NO: 240 - protein, 9 aa, synthetic construct
  REGION 1..9: note = Synthetic Construct
  VARIANT 5: note = Xaa is Ser, Arg, Leu, Glu, or Phe
QQWYXNPLT

SEQ ID NO: 241 - protein, 10 aa, synthetic construct
  REGION 1..10: note = Synthetic Construct
SASSSISYSH

SEQ ID NO: 242 - protein, 12 aa, synthetic construct
  REGION 1..12: note = Synthetic Construct
GFSLRKVGRSVS

SEQ ID NO: 243 - protein, 16 aa, synthetic construct
  REGION 1..16: note = Synthetic Construct
HIYWDDDKRYNPSLRN

SEQ ID NO: 244 - protein, 14 aa, synthetic construct
  REGION 1..14: note = Synthetic Construct
RAITTVIGGGTFDY

SEQ ID NO: 245 - protein, 9 aa, synthetic construct
  REGION 1..9: note = Synthetic Construct
QQWYRNPLT

SEQ ID NO: 246 - protein, 12 aa, synthetic construct
  REGION 1..12: note = Synthetic Construct
GFSLRKVGSSVS

SEQ ID NO: 247 - protein, 16 aa, synthetic construct
  REGION 1..16: note = Synthetic Construct
HIYWDDDKRLNPSLRN

SEQ ID NO: 248 - protein, 14 aa, synthetic construct
  REGION 1..14: note = Synthetic Construct
RAITTVIGGGTFDL

SEQ ID NO: 249 - protein, 9 aa, synthetic construct
  REGION 1..9: note = Synthetic Construct
QQWYLNPLT

SEQ ID NO: 250 - protein, 119 aa, synthetic construct
  REGION 1..119: note = Synthetic Construct
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARDRGYSYGLDYWGQGTLVTVSS

SEQ ID NO: 251 - protein, 107 aa, synthetic construct
  REGION 1..107: note = Synthetic Construct
DIQMTQSPSSLSASVGDRVTITCRASQGIRNALGWYQQKPGKAPQRLIYAASSLQSGVPSRFSGSGSGTEFTLTISSLQPEDFATYYCLQHNSYPRTFGQGTKVEIK

SEQ ID NO: 252 - protein, 123 aa, synthetic construct
  REGION 1..123: note = Synthetic Construct
QVQLVQSGAEVKKPGASVKVSCKASGYTFTGYYMHWVRQAPGQGLEWMGWINPNNGGTNYAQKFQGRVTMTRDTSISTAYMELSRLRSDDTAVYHCARNTVGSGYYYYGMDVWGQGTTVTVSS

SEQ ID NO: 253 - protein, 112 aa, synthetic construct
  REGION 1..112: note = Synthetic Construct
DIVMTQSPLSLPVTPGEPASISCRSSQSLLHSNGYNYLDWYLQKSGQSPQLLIHLGSNRASGVPDRFSGSGSGTDFTLKISRVEAEDVGVYYCMQVLQTPLTFGGGTKVEIK

SEQ ID NO: 254 - protein, 119 aa, synthetic construct
  REGION 1..119: note = Synthetic Construct
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARDRGYSYGIDYWGQGTLVTVSS

SEQ ID NO: 255 - protein, 107 aa, synthetic construct
  REGION 1..107: note = Synthetic Construct
DIQMTQSPSSLSASVGDRVTITCRASQGIRNALGWYQQKPGKAPKRLIYAASSLQSGVPSRFSGSGSGTEFTLTISSLQPEDFATYYCLQHNSYPRTFGQGTKVEIK

SEQ ID NO: 256 - protein, 119 aa, synthetic construct
  REGION 1..119: note = Synthetic Construct
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARDRGYYYGMDVWGQGTTVTVSS

SEQ ID NO: 257 - protein, 107 aa, synthetic construct
  REGION 1..107: note = Synthetic Construct
DIQMTQSPSSLSASVGDRVTLTCRASQGIRNALGWYQQKPGKAPKRLIYGASSLQSGVPSRFSGSGSGTEFTLTISSLQPEDFATYYCLQHNSYPFTFGPGTKVDIK

SEQ ID NO: 258 - protein, 119 aa, synthetic construct
  REGION 1..119: note = Synthetic Construct
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARDRGSYFLFDYWGQGTLVTVSS

SEQ ID NO: 259 - protein, 107 aa, synthetic construct
  REGION 1..107: note = Synthetic Construct
DMQMTQSPSSLSASVGDRVTITCRASQGIRNDLGWYQQKPGKAPKRLIYGASSLQSGVPSRFSGSGSGTEFTLTISSLQPEDSATYYCLQHNSYPLTFGGGTKVEIK

SEQ ID NO: 260 - protein, 119 aa, synthetic construct
  REGION 1..119: note = Synthetic Construct
EVQLVESGGGLVKPGGSLRLSCAASGFTFSSYSMNWVRQAPGKGLEWVSSISSSSSYIYYADSVKGRFTISRDNAKNSLYLQMNSLRAEDTTVYYCARDRGYLYGMDVWGQGTTVTVSS

SEQ ID NO: 261 - protein, 107 aa, synthetic construct
  REGION 1..107: note = Synthetic Construct
DIQMTQSPSSLSASVGDRVTITCRASQAIRNDLGWYQQKPGKAPKRLIYAASSLQSGVPSRFSGSGSGTEFTLTISSLQPEDFATYYCLQHNSYPRTFGQGTKVEIK

SEQ ID NO: 262 - protein, 123 aa, synthetic construct
  REGION 1..123: note = Synthetic Construct
QVQLVQSGAEVKKPGASVKVSCKASGYTFTGYYMHWVRQAPGQGLEWMGWINPNSGGTNYAQKFQGRVTMTRDTSISTAYLELSRLRSDDTAVYYCARNTVGTGYYYYGMDVWGQGTTVTVSS

SEQ ID NO: 263 - protein, 112 aa, synthetic construct
  REGION 1..112: note = Synthetic Construct
DIVMTQSPLSLPVTPGEPASISCRSSQSLLHSNGYNYLDWYLQKPGQSPQVLIYLVSNRASGVPDRFSGSGSGTDFTLKISRVEAEDVGVYYCMQALQTPPTFGGGTKVEIK

SEQ ID NO: 264 - protein, 119 aa, synthetic construct; identical to SEQ ID NO: 250
  REGION 1..119: note = Synthetic Construct

SEQ ID NO: 265 - protein, 107 aa, synthetic construct
  REGION 1..107: note = Synthetic Construct
DIQMTQSPSSLSASVGDRVTITCRASQGIRNDLGWYQQKPGKAPKRLIYAASSLQSGVPSRFSGSGSGTEFTLTISSLQPEDFATYYCLQLNSYPFTFGPGTKVDIK

SEQ ID NO: 266 - protein, 122 aa, synthetic construct
  REGION 1..122: note = Synthetic Construct
EVQLLESGGGLIQPGGSLRLSCAASGFTFSSFAMSWVRQAPGKGLEWVSTISGSGGYTFYADSVKGRFTISRDNSKNTLYLQMNSLRAEDTAVYYCAKDGRYNWNYGAFDIWGQGTMVTVSS

SEQ ID NO: 267 - protein, 107 aa, synthetic construct
  REGION 1..107: note = Synthetic Construct
EIVMTQSPATLSVSPGERATLSCRASQSVSSNLAWYQQKPGQAPRLLIYGASTRATGFPARFSGSGSGTEFTLTISSLQSEDFAIYYCQQYNNWPLTFGGGTKVEIK

SEQ ID NO: 268 - protein, 122 aa, synthetic construct
  REGION 1..122: note = Synthetic Construct
EVQLLESGGGLIQPGGSLRLSCAASGFTFSSFAMSWVRQAPGKGLEWVSTISGSGGSTYYADFVKGRFTISRDNSKNTLYLQMNSLRAEDTAVYYCAKDGRYNWNYGAFDIWGQGTMVTVSS

SEQ ID NO: 269 - protein, 107 aa, synthetic construct
  REGION 1..107: note = Synthetic Construct
EIVMTQSPATLSVSPGERATLSCRASQSVRSNLAWFQQKPGQAPRLLIYGASTRATGIPARFSGSGSGTEFTLTISSLQSEDFAVYYCQQYNNWPLTFGGGTKVEIK

SEQ ID NO: 270 - protein, 122 aa, synthetic construct
  REGION 1..122: note = Synthetic Construct
EVQLLESGGGLVQPGGSLRLSCAASGFTFSSYAMSWVRQAPGKGLEWVSAISGSGGSTFYADSVTGRFTISRDNSKNTLFLQLNSLRAEDTAVYHCAKDGRFNWNYGASDIWGQGTMVTVSS

SEQ ID NO: 271 - protein, 107 aa, synthetic construct
  REGION 1..107: note = Synthetic Construct
EIVMTQSPATLSVSPGERATLSCRTSQSVSSNLAWYQQKPGQAPRLLIYGASTRATGIPARFSGSGSGTEFTLTISSLQSEDFAVYYCQQYNNWPLTFGGGTKVEIK

SEQ ID NO: 272 - protein, 122 aa, synthetic construct
  REGION 1..122: note = Synthetic Construct
EVQLLESGGGLVQPGGSLRLSCAASGFTFSSFAMSWVRQAPGKGLEWVSTISGSGGYTFYADSVKGRFTISRDNSKNTLYLQMNSLRAEDTAVYYCAKDGRYNWNYGAFDIWGQGTMVTVSS

SEQ ID NO: 273 - protein, 107 aa, synthetic construct
  REGION 1..107: note = Synthetic Construct
EIVMTQSPATLSVSPGERATLSCRASQSVSSNLAWYQQKPGQAPRLLIYGASTRATGIPARFSGSGSGTEFTLTISSLQSEDFAVYYCQQYNNWPLTFGGGTKVEIK

SEQ ID NO: 274 - protein, 445 aa, synthetic construct
  REGION 1..445: note = Synthetic Construct
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARDRGYSYGLDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 275 - protein, 214 aa, synthetic construct
  REGION 1..214: note = Synthetic Construct
DIQMTQSPSSLSASVGDRVTITCRASQGIRNALGWYQQKPGKAPQRLIYAASSLQSGVPSRFSGSGSGTEFTLTISSLQPEDFATYYCLQHNSYPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 276 - protein, 449 aa, synthetic construct
  REGION 1..449: note = Synthetic Construct
QVQLVQSGAEVKKPGASVKVSCKASGYTFTGYYMHWVRQAPGQGLEWMGWINPNNGGTNYAQKFQGRVTMTRDTSISTAYMELSRLRSDDTAVYHCARNTVGSGYYYYGMDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 277 - protein, 219 aa, synthetic construct
  REGION 1..219: note = Synthetic Construct
DIVMTQSPLSLPVTPGEPASISCRSSQSLLHSNGYNYLDWYLQKSGQSPQLLIHLGSNRASGVPDRFSGSGSGTDFTLKISRVEAEDVGVYYCMQVLQTPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 278 - protein, 445 aa, synthetic construct
  REGION 1..445: note = Synthetic Construct
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARDRGYSYGIDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 279 - protein, 214 aa, synthetic construct
  REGION 1..214: note = Synthetic Construct
DIQMTQSPSSLSASVGDRVTITCRASQGIRNALGWYQQKPGKAPKRLIYAASSLQSGVPSRFSGSGSGTEFTLTISSLQPEDFATYYCLQHNSYPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 280 - protein, 445 aa, synthetic construct
  REGION 1..445: note = Synthetic Construct
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARDRGYYYGMDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 281 - protein, 214 aa, synthetic construct
  REGION 1..214: note = Synthetic Construct
DIQMTQSPSSLSASVGDRVTLTCRASQGIRNALGWYQQKPGKAPKRLIYGASSLQSGVPSRFSGSGSGTEFTLTISSLQPEDFATYYCLQHNSYPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 282 - protein, 445 aa, synthetic construct
  REGION 1..445: note = Synthetic Construct
EVQLVESGGGLVKPGGSLRLSCAASGFTFSSYSMNWVRQAPGKGLEWVSSISSSSSYIYYADSVKGRFTISRDNAKNSLYLQMNSLRAEDTTVYYCARDRGYLYGMDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 283 - protein, 214 aa, synthetic construct
  REGION 1..214: note = Synthetic Construct
DIQMTQSPSSLSASVGDRVTITCRASQAIRNDLGWYQQKPGKAPKRLIYAASSLQSGVPSRFSGSGSGTEFTLTISSLQPEDFATYYCLQHNSYPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 284 - protein, 449 aa, synthetic construct
  REGION 1..449: note = Synthetic Construct
QVQLVQSGAEVKKPGASVKVSCKASGYTFTGYYMHWVRQAPGQGLEWMGWINPNSGGTNYAQKFQGRVTMTRDTSISTAYLELSRLRSDDTAVYYCARNTVGTGYYYYGMDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 285 - protein, 219 aa, synthetic construct
  REGION 1..219: note = Synthetic Construct
DIVMTQSPLSLPVTPGEPASISCRSSQSLLHSNGYNYLDWYLQKPGQSPQVLIYLVSNRASGVPDRFSGSGSGTDFTLKISRVEAEDVGVYYCMQALQTPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 286 - protein, 449 aa, synthetic construct
  REGION 1..449: note = Synthetic Construct
EVQLLESGGGLIQPGGSLRLSCAASGFTFSSFAMSWVRQAPGKGLEWVSTISGSGGYTFYADSVKGRFTISRDNSKNTLYLQMNSLRAEDTAVYYCAKDGRYNWNYGAFDIWGQGTMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 287 - protein, 214 aa, synthetic construct
  REGION 1..214: note = Synthetic Construct
EIVMTQSPATLSVSPGERATLSCRASQSVSSNLAWYQQKPGQAPRLLIYGASTRATGFPARFSGSGSGTEFTLTISSLQSEDFAIYYCQQYNNWPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 288 - protein, 445 aa, synthetic construct
  REGION 1..445: note = Synthetic Construct
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARDRGSYFLFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 289 - protein, 214 aa, synthetic construct
  REGION 1..214: note = Synthetic Construct
DMQMTQSPSSLSASVGDRVTITCRASQGIRNDLGWYQQKPGKAPKRLIYGASSLQSGVPSRFSGSGSGTEFTLTISSLQPEDSATYYCLQHNSYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 290 - protein, 445 aa, synthetic construct; identical to SEQ ID NO: 274
  REGION 1..445: note = Synthetic Construct

SEQ ID NO: 291 - protein, 214 aa, synthetic construct
  REGION 1..214: note = Synthetic Construct
DIQMTQSPSSLSASVGDRVTITCRASQGIRNDLGWYQQKPGKAPKRLIYAASSLQSGVPSRFSGSGSGTEFTLTISSLQPEDFATYYCLQLNSYPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 292 - protein, 449 aa, synthetic construct
  REGION 1..449: note = Synthetic Construct
EVQLLESGGGLIQPGGSLRLSCAASGFTFSSFAMSWVRQAPGKGLEWVSTISGSGGSTYYADFVKGRFTISRDNSKNTLYLQMNSLRAEDTAVYYCAKDGRYNWNYGAFDIWGQGTMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 293 - protein, 214 aa, synthetic construct
  REGION 1..214: note = Synthetic Construct
EIVMTQSPATLSVSPGERATLSCRASQSVRSNLAWFQQKPGQAPRLLIYGASTRATGIPARFSGSGSGTEFTLTISSLQSEDFAVYYCQQYNNWPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 294 - protein, 449 aa, synthetic construct
  REGION 1..449: note = Synthetic Construct
EVQLLESGGGLVQPGGSLRLSCAASGFTFSSYAMSWVRQAPGKGLEWVSAISGSGGSTFYADSVTGRFTISRDNSKNTLFLQLNSLRAEDTAVYHCAKDGRFNWNYGASDIWGQGTMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 295 - protein, 214 aa, synthetic construct
  REGION 1..214: note = Synthetic Construct
EIVMTQSPATLSVSPGERATLSCRTSQSVSSNLAWYQQKPGQAPRLLIYGASTRATGIPARFSGSGSGTEFTLTISSLQSEDFAVYYCQQYNNWPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 296 - protein, 448 aa, synthetic construct
  REGION 1..448: note = Synthetic Construct
EVQLLESGGGLVQPGGSLRLSCAASGFTFSSFAMSWVRQAPGKGLEWVSTISGSGGYTFYADSVKGRFTISRDNSKNTLYLQMNSLRAEDTAVYYCAKDGRYNWNYGAFDIWGQGTMVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 297 - protein, 214 aa, synthetic construct
  REGION 1..214: note = Synthetic Construct
EIVMTQSPATLSVSPGERATLSCRASQSVSSNLAWYQQKPGQAPRLLIYGASTRATGIPARFSGSGSGTEFTLTISSLQSEDFAVYYCQQYNNWPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 298 - protein, 115 aa, synthetic construct
  REGION 1..115: note = Synthetic Construct
EVQLVESGGGLVQPGGSLRLSCAASGFTFSRYWMNWVRQAPGKGLEWVAQIRLKSDNYATHYAESVKGRFTISRDNAKNSLYLQMNSLRAEDTAVYYCTEGLDYWGQGTTVTVSS

SEQ ID NO: 299 - protein, 107 aa, synthetic construct
  REGION 1..107: note = Synthetic Construct
DIQMTQSPSSLSASVGDRVTITCKASQDINKYVAWYQQKPGKAPKLLIYYTSWLQPGVPSRFSGSGSGTDFTFTISSLQPEDIATYYCLQYDNLLYTFGQGTKLEIK

SEQ ID NO: 300 - protein, 5 aa, synthetic construct
  REGION 1..5: note = Synthetic Construct
RYWMN

SEQ ID NO: 301 - protein, 19 aa, synthetic construct
  REGION 1..19: note = Synthetic Construct
QIRLKSDNYATHYAESVKG

SEQ ID NO: 302 - protein, 4 aa, synthetic construct
  REGION 1..4: note = Synthetic Construct
GLDY

SEQ ID NO: 303 - protein, 11 aa, synthetic construct
  REGION 1..11: note = Synthetic Construct
KASQDINKYVA

SEQ ID NO: 304 - protein, 7 aa, synthetic construct
  REGION 1..7: note = Synthetic Construct
YTSWLQP

SEQ ID NO: 305 - protein, 9 aa, synthetic construct
  REGION 1..9: note = Synthetic Construct
LQYDNLLYT

SEQ ID NO: 306 - protein, 126 aa, synthetic construct
  REGION 1..126: note = Synthetic Construct
QIQLVQSGGGVVQPGRSLRLSCAASGFTFSSYGMHWVRQAPGKGLEWVAVISYDGSNKYYADSVKGRFTISRDNSKNTLYLQMNSLRAEDTAVYYCARDLLVRFLEWSHYYGMDVWGQGTTVTVSS

SEQ ID NO: 307 - protein, 109 aa, synthetic construct
  REGION 1..109: note = Synthetic Construct
QPVLTQPPSASGTPGQRVTISCSGSSSNIGSNTVHWYQQLPGTAPKLLIYSDNQRPSGVPDRFSGSKSGTSASLVISGLQSDDEADYYCAAWDDSLNGVFGGGTKLTVL

SEQ ID NO: 308 - protein, 126 aa, synthetic construct
  REGION 1..126: note = Synthetic Construct
QVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCARDLLVRFLEWSHYYGMDVWGQGTTVTVSS

SEQ ID NO: 309 - protein, 109 aa, synthetic construct
  REGION 1..109: note = Synthetic Construct
QSVLTQPPSASGTPGQRVTISCSGSSSNIGSNTVHWYQQLPGTAPKLLIYSDNQRPSGVPDRFSGSKSGTSASLAISGLQSEDEADYYCAAWDDSLNGVFGGGTKLTVL

SEQ ID NO: 310 - protein, 131 aa, synthetic construct
  REGION 1..131: note = Synthetic Construct
QVQLQQSGGGLVQPGGSLRLSCAASGFTFSSYAMTWVRQAPGKGLEWVSAISGGGGATYYADSVEGRFTISRDNSKNTLYLQMNSLRAEDTAIYYCASPAGHICSGGSCQYYYYGMDVWGQGTTVTVSSGS

SEQ ID NO: 311 - protein, 116 aa, synthetic construct
  REGION 1..116: note = Synthetic Construct
DIQMTQSPSFLSASVGDRVTITCRASQGISSYLAWYQQKPGKAPKLLIYAASSLQSGVPSRFSGSGSGTDFTLTISSLQPEDFATYYCQQSYSTPLTFGGGTRVDMARTVAAPSVF

SEQ ID NO: 312 - protein, 128 aa, synthetic construct
  REGION 1..128: note = Synthetic Construct
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARDRHLYRRGYSSVDGMDVWGQGTTVTVSSGS

SEQ ID NO: 313 - protein, 117 aa, synthetic construct
  REGION 1..117: note = Synthetic Construct
EIVMTQSPGTLSLSPGERATLSCRASQSVSSGYLAWYQQKAGQAPRLLIYGGSARAPGIPARFSGSGSGTDFTLTISSLEPEDFAVYYCQQRSIWPLTFGGGTKVEIKRTVAAPSVF

SEQ ID NO: 314 - protein, 120 aa, synthetic construct
  REGION 1..120: note = Synthetic Construct
QVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARHPIGSGYDYWGQGTLVTVSSGS

SEQ ID NO: 315 - protein, 121 aa, synthetic construct
  REGION 1..121: note = Synthetic Construct
DIVMTQSPVSLPVTLGQPASISCRSSQSVVYRDGNTYLSWFLQRPGQSPRRLIYKVSNRDSGVPDRFSGSGSATDFTLKISRVEAEDVGVYYCMQGSSWPYTFGQGTKLEIKRTVAAPSVF

SEQ ID NO: 316 - protein, 122 aa, synthetic construct
  REGION 1..122: note = Synthetic Construct
QVQLVQSGGGVVRPGGSLRLSCAASGFTFDDYGMSWVRQAPGKGLEWVSGISWNSGSIGYADSVKGRFTISRDNAKNSLYLQMNSLRAEDTALYYCAKEISDTAMGFDYWGQGTLVTVSSGS

SEQ ID NO: 317 - protein, 126 aa, synthetic construct
  REGION 1..126: note = Synthetic Construct
QSALTQPASVSGSPGQSITISCTGTSSDVGGYNYISWYQQYPGKAPKLMIYDVSKRPSGVSNRFSGSKSGNTASLTISGLQAEDEADYYCNSYTSSRTVVFGGGTKLTVLGQPKAAPSVTLFPPSS

SEQ ID NO: 318 - protein, 121 aa, synthetic construct
  REGION 1..121: note = Synthetic Construct
QVQLVQSGGGLVQPGGSLRLSCAASGFTFSNYWMYWVRQAPGKGLVWVSNINSDGSSTSYADSVKGRFTISRDNAKNSLYLQMNSLRDEDTAVYYCAREGYNAHYFDYWGQGTLVTVSSGS

SEQ ID NO: 319 - protein, 131 aa, synthetic construct
  REGION 1..131: note = Synthetic Construct
QSALTQPASVSGSPGQSITISCTGTSSDVGGYNYVSWYQQHPGKAPKLLIYDVTKRPSGVSNRFSGSKSGNTASLTISGLQAEDEADYYCSSYTSSSTVVFGGGTKLTVLGQPKAAPSVTLFPPSSKASGA

SEQ ID NO: 320 - protein, 121 aa, synthetic construct
  REGION 1..121: note = Synthetic Construct
QIQLVQSGGGVVQPGRSLRLSCAASGFTFSSYGMHWVRQAPGKGLEWVAVIWYDGSNKYYADSVKGRFTISRDNSKNTLYLQMNSLRAEDTAVYYCAKGLGYHPLFDYWGQGTLVTVSSGS

SEQ ID NO: 321 - protein, 126 aa, synthetic construct
  REGION 1..126: note = Synthetic Construct
DIVMTQSPDSLAVSLGERATINCKSSQSVLYSSDNKNYLAWYQQKPGQPPKLLIYWASTRESGVPDRFSGSGSGTDFTLTISSLQAEDVAVYYCQQYYSTPSFGPGTKVDIKRTVAAPSVFKASGA

SEQ ID NO: 322 - protein, 6 aa, synthetic construct
  REGION 1..6: note = Synthetic Construct
SSYGMH

SEQ ID NO: 323 - protein, 17 aa, synthetic construct
  REGION 1..17: note = Synthetic Construct
VISYDGSNKYYADSVKG

SEQ ID NO: 324 - protein, 17 aa, synthetic construct
  REGION 1..17: note = Synthetic Construct
DLLVRFLEWSHYYGMDV

SEQ ID NO: 325 - protein, 13 aa, synthetic construct
  REGION 1..13: note = Synthetic Construct
SGSSSNIGSNTVH

SEQ ID NO: 326 - protein, 7 aa, synthetic construct
  REGION 1..7: note = Synthetic Construct
SDNQRPS

SEQ ID NO: 327 - protein, 10 aa, synthetic construct
  REGION 1..10: note = Synthetic Construct
AAWDDSLNGV

SEQ ID NO: 328 - protein, 10 aa, synthetic construct
  REGION 1..10: note = Synthetic Construct
GFTFSSYGMH

SEQ ID NO: 329 - protein, 7 aa, synthetic construct
  REGION 1..7: note = Synthetic Construct
ISYDGSN

SEQ ID NO: 330 - protein, 8 aa, synthetic construct
  REGION 1..8: note = Synthetic Construct
SSNIGSNT

SEQ ID NO: 331 - skipped

SEQ ID NO: 332 - protein, 8 aa, synthetic construct
  REGION 1..8: note = Synthetic Construct
GFTFSSYA

SEQ ID NO: 333 - protein, 8 aa, synthetic construct
  REGION 1..8: note = Synthetic Construct
GFTFDDYG

SEQ ID NO: 334 - protein, 8 aa, synthetic construct
  REGION 1..8: note = Synthetic Construct
GFTFSNYW

SEQ ID NO: 335 - protein, 8 aa, synthetic construct
  REGION 1..8: note = Synthetic Construct
ISGGGGAT

SEQ ID NO: 336 - protein, 8 aa, synthetic construct
  REGION 1..8: note = Synthetic Construct
INSDGSST

SEQ ID NO: 337 - protein, 22 aa, synthetic construct
  REGION 1..22: note = Synthetic Construct
ASPAGHICSGGSCQYYYYGMDV

SEQ ID NO: 338 - protein, 19 aa, synthetic construct
  REGION 1..19: note = Synthetic Construct
ARDRHLYRRGYSSVDGMDV

SEQ ID NO: 339 - protein, 11 aa, synthetic construct
  REGION 1..11: note = Synthetic Construct
ARHPIGSGYDY

SEQ ID NO: 340 - protein, 13 aa, synthetic construct
  REGION 1..13: note = Synthetic Construct
AKEISDTAMGFDY

SEQ ID NO: 341 - protein, 12 aa, synthetic construct
  REGION 1..12: note = Synthetic Construct
AREGYNAHYFDY

SEQ ID NO: 342 - protein, 12 aa, synthetic construct
  REGION 1..12: note = Synthetic Construct
AKGLGYHPLFDY

SEQ ID NO: 343 - protein, 6 aa, synthetic construct
  REGION 1..6: note = Synthetic Construct
QGISSY

SEQ ID NO: 344 - protein, 7 aa, synthetic construct
  REGION 1..7: note = Synthetic Construct
QSVSSGY

SEQ ID NO: 345 - protein, 11 aa, synthetic construct
  REGION 1..11: note = Synthetic Construct
QSVVYRDGNTY

SEQ ID NO: 346 - protein, 12 aa, synthetic construct
  REGION 1..12: note = Synthetic Construct
QSVLYSSDNKNY

SEQ ID NO: 347-349 - skipped (3 entries)

SEQ ID NO: 350 - protein, 9 aa, synthetic construct
  REGION 1..9: note = Synthetic Construct
QQSYSTPLT

SEQ ID NO: 351 - protein, 9 aa, synthetic construct
  REGION 1..9: note = Synthetic Construct
QQRSIWPLT

SEQ ID NO: 352 - protein, 9 aa, synthetic construct
  REGION 1..9: note = Synthetic Construct
MQGSSWPYT

SEQ ID NO: 353 - protein, 10 aa, synthetic construct
  REGION 1..10: note = Synthetic Construct
NSYTSSRTVV

SEQ ID NO: 354 - protein, 10 aa, synthetic construct
  REGION 1..10: note = Synthetic Construct
SSYTSSSTVV

SEQ ID NO: 355 - protein, 8 aa, synthetic construct
  REGION 1..8: note = Synthetic Construct
QQYYSTPS

SEQ ID NO: 356 - protein, 116 aa, synthetic construct
  REGION 1..116: note = Synthetic Construct
EVQLLESGGGLVQPGGSLRLSCAASGFTFSSYAMSWVRQAPGKGLEWVSTISSGGSYTSYPDSVKGRFTISRDNSKNTLYLQMNSLRAEDTAVYYCAKQDYAMNYWGQGTLVTVSS

SEQ ID NO: 357 - protein, 107 aa, synthetic construct
  REGION 1..107: note = Synthetic Construct
DIQMTQSPSSLSASVGDRVTITCKASQDVSTAVAWYQQKPGKAPKLLIYSASYRYTGVPSRFSGSGSGTDFTLTISSLQPEDFATYYCQQHYSTPWTFGGGTKVEIK

SEQ ID NO: 358 - protein, 107 aa, synthetic construct
  REGION 1..107: note = Synthetic Construct
DIQMTQSPSSLSASVGDRVTITCKASQDVSTAVAWYQQKPGKAPKLLIYSASYRYTGVPSRFSGSGSGTDFTLTISSLQPEDFATYYCQQHYSTPWTFGQGTKVEIK

SEQ ID NO: 359 - protein, 5 aa, synthetic construct
  REGION 1..5: note = Synthetic Construct
SYAMS

SEQ ID NO: 360 - protein, 17 aa, synthetic construct
  REGION 1..17: note = Synthetic Construct
TISSGGSYTSYPDSVKG

SEQ ID NO: 361 - protein, 7 aa, synthetic construct
  REGION 1..7: note = Synthetic Construct
QDYAMNY

SEQ ID NO: 362 - protein, 11 aa, synthetic construct
  REGION 1..11: note = Synthetic Construct
KASQDVSTAVA

SEQ ID NO: 363 - protein, 7 aa, synthetic construct
  REGION 1..7: note = Synthetic Construct
SASYRYT

SEQ ID NO: 364 - protein, 9 aa, synthetic construct
  REGION 1..9: note = Synthetic Construct
QQHYSTPWT

SEQ ID NO: 365 - protein, 10 aa, synthetic construct
  REGION 1..10: note = Synthetic Construct
TISSGGSYTS

SEQ ID NO: 366 - protein, 10 aa, synthetic construct
  REGION 1..10: note = Synthetic Construct
GFTFSSYAMS

SEQ ID NO: 367 - protein, 330 aa, synthetic construct
  REGION 1..330: note = Synthetic Construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 368 - protein, 107 aa, synthetic construct [sequence omitted]

SEQ ID NO: 369 - protein, 446 aa, synthetic construct
  REGION 1..446: note = Synthetic Construct
EVQLLESGGGLVQPGGSLRLSCAASGFTFSSYAMSWVRQAPGKGLEWVSTISSGGSYTSYPDSVKGRFTISRDNSKNTLYLQMNSLRAEDTAVYYCAKQDYAMNYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 370 - protein, 214 aa, synthetic construct
  REGION 1..214: note = Synthetic Construct
DIQMTQSPSSLSASVGDRVTITCKASQDVSTAVAWYQQKPGKAPKLLIYSASYRYTGVPSRFSGSGSGTDFTLTISSLQPEDFATYYCQQHYSTPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 371 - protein, 120 aa, synthetic construct
  REGION 1..120: note = Synthetic Construct
QVQLVQSGAEVKKPGASVKVSCKASGYTFTSYWMQWVRQAPGQGLEWMGAIYPGDGDTRYTQKFKGRVTMTRDTSTSTVYMELSSLRSEDTAVYYCARMGGYDRYYFDYWGQGTLVTVSS

SEQ ID NO: 372 - protein, 113 aa, synthetic construct
  REGION 1..113: note = Synthetic Construct
DIVMTQSPDSLAVSLGERATINCKSSQSLLNSANQKNYLAWYQQKPGQPPKLLIYFASTRESGVPDRFSGSGSGTDFTLTISSLQAEDVAVYYCQQHYNTPLTFGGGTKVEIK

SEQ ID NO: 373 - protein, 120 aa, synthetic construct
  REGION 1..120: note = Synthetic Construct
QVQLQQSGAELARPGASVKLSCKASGYTFTSYWMQWVKQRPGQGLEWIGAIYPGDGDTRYTQKFKGKATLTADKSSSTAYMQLSSLASEDSAVYYCARMGGYDRYYFDYWGQGTTLTVSS

SEQ ID NO: 374 - protein, 113 aa, synthetic construct
  REGION 1..113: note = Synthetic Construct
DIVMTQSPSSLAMSVGQKVTMSCKSSQSLLNSANQKNYLAWYQQKPGQSPKLLVYFASTRESGVPDRFIGSGSGTDFTLTISSVQAEDLADYFCQQHYNTPLTFGAGTKLELK

SEQ ID NO: 375 - protein, 5 aa, synthetic construct
  REGION 1..5: note = Synthetic Construct
SYWMQ

SEQ ID NO: 376 - protein, 17 aa, synthetic construct
  REGION 1..17: note = Synthetic Construct
AIYPGDGDTRYTQKFKG

SEQ ID NO: 377 - protein, 13 aa, synthetic construct
  REGION 1..13: note = Synthetic Construct
ARMGGYDRYYFDY

SEQ ID NO: 378 - protein, 17 aa, synthetic construct
  REGION 1..17: note = Synthetic Construct
KSSQSLLNSANQKNYLA

SEQ ID NO: 379 - protein, 7 aa, synthetic construct
  REGION 1..7: note = Synthetic Construct
FASTRES

SEQ ID NO: 380 - protein, 9 aa, synthetic construct
  REGION 1..9: note = Synthetic Construct
QQHYNTPLT

SEQ ID NO: 381 - protein, 10 aa, synthetic construct
  REGION 1..10: note = Synthetic Construct
GYTFTSYWMQ

SEQ ID NO: 382 - protein, 9 aa, synthetic construct
  REGION 1..9: note = Synthetic Construct
AIYPGDGDT

SEQ ID NO: 383 - protein, 13 aa, synthetic construct
  REGION 1..13: note = Synthetic Construct
ARMGGYDRYYFDY

SEQ ID NO: 384 - protein, 17 aa, synthetic construct
  REGION 1..17: note = Synthetic Construct
KSSQSLLNSANQKNYLA

SEQ ID NO: 385 - protein, 7 aa, synthetic construct
  REGION 1..7: note = Synthetic Construct
FASTRES

SEQ ID NO: 386 - protein, 9 aa, synthetic construct
  REGION 1..9: note = Synthetic Construct
QQHYNTPLT

SEQ ID NO: 387 - protein, 118 aa, synthetic construct
  REGION 1..118: note = Synthetic Construct
QVQLVESGGGVVQPGRSLRLSCAASGFTFSRYGIHWVRQAPGKGLEWVAVISYDGSDEYYVDSVKGRFSISRDNSKNTLYLQMNSLRPADSAVYYCVKGDLELGFDYWGQGTLVTVSS

SEQ ID NO: 388 - protein, 112 aa, synthetic construct
  REGION 1..112: note = Synthetic Construct
DIVMTQAAPSIPVIPGESVSMSCRSSKSLLYSNGHTYVYWFVQRPGQSPQLLIYRMSNLASGVPDRFSGSGSGTAFTLRISRVEAEDVGVYYCMQNLEFPLTFGAGTKLELK

SEQ ID NO: 389 - protein, 120 aa, synthetic construct; identical to SEQ ID NO: 164
  REGION 1..120: note = Synthetic Construct

SEQ ID NO: 390 - protein, 107 aa, synthetic construct; identical to SEQ ID NO: 165
  REGION 1..107: note = Synthetic Construct

SEQ ID NO: 391 - protein, 121 aa, synthetic construct
  REGION 1..121: note = Synthetic Construct
QVQLVESGGGVVQPGKSLRLSCAASGFTFSSFGMHWVRQAPGKGLEWVAVIGYDGGNEYYADSVKGRFTISRDNSKNTLNLQMSSLRAEDTAVYYCSTISHYDILSGMDVWGRGTTVTVSS

SEQ ID NO: 392 - protein, 108 aa, synthetic construct
  REGION 1..108: note = Synthetic Construct
DIQMTQSPSSVSASVGDRVTITCRASQGISNWLAWYQQKPGKAPKLLIFAASSLQSGVPSRFSGSASGTDFTLTINSLQPEDFATYYCQQANSFPLTFGGGTKVEIKR

SEQ ID NO: 393 - protein, 8 aa, synthetic construct
  REGION 1..8: note = Synthetic Construct
GFTFSAYA

SEQ ID NO: 394 - protein, 8 aa, synthetic construct
  REGION 1..8: note = Synthetic Construct
ISGSGGSA

SEQ ID NO: 395 - protein, 13 aa, synthetic construct
  REGION 1..13: note = Synthetic Construct
AKDGAWKMSGLDV

SEQ ID NO: 396 - protein, 6 aa, synthetic construct
  REGION 1..6: note = Synthetic Construct
QDISDY

SEQ ID NO: 397 - skipped

SEQ ID NO: 398 - protein, 9 aa, synthetic construct
  REGION 1..9: note = Synthetic Construct
QKYDSAPLT

SEQ ID NO: 399 - protein, 8 aa, synthetic construct
  REGION 1..8: note = Synthetic Construct
GFTFSSFG

SEQ ID NO: 400 - protein, 8 aa, synthetic construct
  REGION 1..8: note = Synthetic Construct
IGYDGGNE

SEQ ID NO: 401 - protein, 14 aa, synthetic construct
  REGION 1..14: note = Synthetic Construct
STISHYDILSGMDV

SEQ ID NO: 402 - protein, 6 aa, synthetic construct
  REGION 1..6: note = Synthetic Construct
QGISNW

SEQ ID NO: 403 - skipped

SEQ ID NO: 404 - protein, 9 aa, synthetic construct
  REGION 1..9: note = Synthetic Construct
QQANSFPLT

SEQ ID NO: 405 - protein, 122 aa, synthetic construct
  REGION 1..122: note = Synthetic Construct
QVQLVQSGGGLVQPGGSLRLSCAASGFTFNTYAISWVRQAPGKGLEWVSTITGSGYNTYYADSVKGRFTISRDNSKNTLYLQMSSLRAEDTAVFYCAKDSRYNWNYGIFDYWGQGTTVTVSS

SEQ ID NO: 406 - protein, 108 aa, synthetic construct
  REGION 1..108: note = Synthetic Construct
DIQMTQSPATLSVSPGERATLSCRASQSVSSNLAWYHQKPGQAPRLLIYGVSTRATGIPARFSGNGSGTEFTLTISSLQSEDFAVYYCQQHNNWPLTFGGGTKVEIKR

SEQ ID NO: 407 - protein, 125 aa, synthetic construct
  REGION 1..125: note = Synthetic Construct
QVQLVQSGPGLVKPSETLSLTCTVSGGSISNSNYYWGWIRQPPGKGLEWIGTTYYSGTTYYNPSLKSRVTISVDTSKNQFSLKLSSVTAADTAVYYCARDYYDSSGYYYNWFDPWGQGTTVTVSS

SEQ ID NO: 408 - protein, 108 aa, synthetic construct
  REGION 1..108: note = Synthetic Construct
DIQLTQSPSSLSASVGDRVTITCRASQDIRNDLGWYQQKPGKAPKRLIYAASSLQSGVPSRFSGSGSGTEFSLTLSSLQPEDFATYFCLQHHIYPWTFGQGTKLEIKR

SEQ ID NO: 409 - protein, 125 aa, synthetic construct
  REGION 1..125: note = Synthetic Construct
QVQLVESGPGLVKPSETLSLTCTVYGGSISSGNYYWGWIRQPPGKGLEWIGTIYYSGSAYYNPSLKSRVTMSVDTSKNQFSLKLSSVTAADTAVYYCVRDYYDSSGHYYNWFDPWGQGTTVTVSS

SEQ ID NO: 410 - protein, 108 aa, synthetic construct
  REGION 1..108: note = Synthetic Construct
AIQMTQSPSSLSASVGDRVTITCRASQDIRHDLGWYQQKPGKAPKRLIYAASSLQSGVPSRFSGSGSGTEFTLTISSLQPEDFATYYCLQHNTYPWTFGQGTKVEIKR

SEQ ID NO: 411 - protein, 119 aa, synthetic construct
  REGION 1..119: note = Synthetic Construct
QVQLVQSGGGLVKPGGSLRLSCAASGFTFSSYSMNWVRQAPGKGLEWVSSISSSSSYIYYADSVKGRFTISRDNAKNSLYLQMNSLRAEDTAVYYCARDRGYTFGVDYWGQGTTVTVSS

SEQ ID NO: 412 - protein, 108 aa, synthetic construct
  REGION 1..108: note = Synthetic Construct
AIQMTQSPSSLSASVGDRVTITCRASQGIRNDLGWYQQKPGKAPKRLIYAASSLQSGVPSRFSGSGSGTEFTLTISSLQPEDFATYYCLQHNSYPYTFGQGTKVEIKR

SEQ ID NO: 413 - protein, 125 aa, synthetic construct
  REGION 1..125: note = Synthetic Construct
QVQLVQSGPGLVKPSETLSLTCTVSGGSIITYSYYWGWIRQPPGKGLEWIGTIHHSGSTYYNPSLKSRVTISVDTSKNQFSLTLSSVTAADTAVYYCARDYYDSSGYYYNWFDPWGQGTMVTVSS

SEQ ID NO: 414 - protein, 108 aa, synthetic construct
  REGION 1..108: note = Synthetic Construct
AIQMTQSPSSLSASVGDRVTITCRASQGIRNDLGWYQQKPGKAPKRLIYAASSLQSGVPSRFSGSGSGTEFTLTISSLQPEDFATYYCLQHNSSPWTFGQGTKVEIKR

SEQ ID NO: 415 - protein, 123 aa, synthetic construct
  REGION 1..123: note = Synthetic Construct
QVHLKESGPTLVKPTQTLTLTCTFSGFSLSTSGVGVGWIRQPPGKALEWLALIYWNDDKRYSPSLKSRLTITKDTSKNQVVLTMTNMDPVDTATYYCTHTSRYNWHYGFLDYWGQGTTVTVSS

SEQ ID NO: 416 - protein, 108 aa, synthetic construct
  REGION 1..108: note = Synthetic Construct
AIQMTQSPSSLSASVGDRVTITCRASQGISNYLAWFQQKPGKAPKSLIYAASSLQSGVPSKFSGSGSGTDFTLTISSLQPEDFATYYCQQYNSYPLTFGGGTKVEIKR

SEQ ID NO: 417 - protein, 125 aa, synthetic construct
  REGION 1..125: note = Synthetic Construct
EVQLVQLGPGLVKPSETLSLTCTVSGGSISNSNYYWGWIRQPPGKGLEWIGTTYYSGTTYYNPSLKSRVTISVDTSKNQFSLKLSSVTAADTAVYYCARDYYDSSGYYYNWFDPWGQGTTVTVSS

SEQ ID NO: 418 - protein, 108 aa, synthetic construct
  REGION 1..108: note = Synthetic Construct
AIQMTQSPSSLSASVGDRVTITCRASQDIRNDLGWYQQKPGKAPKRLIYAASSLQSGVPSRFSGSGSGTEFSLTLSSLQPEDFATYFCLQHHIYPWTFGQGTKVEIKR

SEQ ID NO: 419 - protein, 118 aa, synthetic construct
  REGION 1..118: note = Synthetic Construct
QVQLVEAGGGVVQPGRSLRLSCAASGFTFSRYGIHWVRQAPGKGLEWVAVISYDGTDEYYADSVKGRFTISRDNSKNTLYLQMNSLRPADSAVYYCAKGDLELGFDYWGQGTLVTVSS

SEQ ID NO: 420 - protein, 107 aa, synthetic construct
  REGION 1..107: note = Synthetic Construct
AIRLTQSPSSVSASVGDRVTITCRASQDISIWLAWYQQSPGKAPKLLINVASRLQSGVPSRFSGSGSGTDFTLTINGLQPEDFVTYYCQQANSFPITFGQGTRLATK

SEQ ID NO: 421 - protein, 119 aa, synthetic construct
  REGION 1..119: note = Synthetic Construct
QVQLVQSGGGLVKPGGSLRLSCAASGFIFNTYTMNWVRQAPGKGLEWVSSITSRGTYIFYSDSLKGRFTISRDNANNSLFLQMNSLRVEDTAVYYCSRDRGYTFGPDYWGQGTLVTVSS

SEQ ID NO: 422 - protein, 107 aa, synthetic construct
  REGION 1..107: note = Synthetic Construct
DIQMTQSPSSLSASVGDRVTITCRASQDIRNDLGWYQQKPGKAPKGLIYAASSLQSGVPSRFSGSGSGTEFTLTISSLQPEDFATYHCLHYDFHPRTFGQGTKVEIK

SEQ ID NO: 423 - protein, 130 aa, synthetic construct
  REGION 1..130: note = Synthetic Construct
QVQLVESGGGVVQPGRSLRLSCAASGFTFSSYAMHWVRQAPGKGLEWVAVIWYDGTNYYYADSVKGRFTISRDNSKNTLYLQMNSLRAEDTAVYYCARDPLYYDILTGYSPDYYYGMDVWGQGTTVTVSS

SEQ ID NO: 424 - protein, 107 aa, synthetic construct
  REGION 1..107: note = Synthetic Construct
EIVMTQSPATLSVSPGERATLSCRASQTFSSNLAWYQQKPGQAPRLLIYGASTRATGIPARFSGSGSGTEFTLTISSLQSEDFAVYYCQQYNKWPLTFGGGTKVEIK

SEQ ID NO: 425 - protein, 122 aa, synthetic construct
  REGION 1..122: note = Synthetic Construct
EVQLLESGGGLVQPGESLRLSCAASGFTFSSYAMNWVRQAPGKGLEWVSTISGSGGYIYYADSVKGRFTISRDNSKNTLYLQMNSLRAEDTAVYFCAKDSRYNWNYGNFDYWGQGTLVTVSS

SEQ ID NO: 426 - protein, 107 aa, synthetic construct
  REGION 1..107: note = Synthetic Construct
EIVMTQSPATLSVSPGERATLSCRASQNVSSNLAWNKQKPGQAPRLLIYATSTRATGVPARFSASGSGTDFALTINSLQSEDFAVYYCQQYNNWPLTFGGGTKVEIK

SEQ ID NO: 427 - protein, 122 aa, synthetic construct
  REGION 1..122: note = Synthetic Construct
EVQLLESGGGLVQPGGSLRLSCVASRFTFSSNAMSWVRQAPGTGLEWVSAITGSGSRTYYADSVKGRFTISRDNSKNTVYLQMNSLRGEDTAVYYCAKDQGGTWNYGDFDYWGQGTLVTVSS

SEQ ID NO: 428 - protein, 107 aa, synthetic construct
  REGION 1..107: note = Synthetic Construct
EIVMTQSPATLSVSPGERATLSCRASQSVSSNLAWYQQKPGQAPRLLIYGASTRATGLPARFSGSGSGTDFTLTISSLQSEDFAVYYCQQYNNWPLTFGGGTKVEIK

SEQ ID NO: 429 - protein, 124 aa, synthetic construct
  REGION 1..124: note = Synthetic Construct
QVQLVESGGDVVQPGRSLRLSCTASGFTFSSYGMHWVRQAPGRGLEWVAVISFDGKNKYYADSVKGRFTVSRDNSKNTLFLQMNSLRAEDTALYYCAKRIAATGYYYFYGLDVWGQGTTVTVSS

SEQ ID NO: 430 - protein, 107 aa, synthetic construct
  REGION 1..107: note = Synthetic Construct
EIMMTQSPATLSVSPGERGTLSCRASQSVSSNLAWYQQKPGQAPRLLIYGASTRASGIPARFSGSGSGTEFTLTVSSLQSEDFAVYYCQQYNNWPLTFGGGTKVEIK

SEQ ID NO: 431 - protein, 122 aa, synthetic construct
  REGION 1..122: note = Synthetic Construct
EVQLLESGGGLVQPGGSLRLSCAASGFTSITYAMSWVRQAPGKGLEWVSAISVSGTNTYYADSVKGRFTISRDKSKNMLYLQMNSLRAEDTAVYYCAKDLLHNWKYGTFDIWGQGTMVTVSS

SEQ ID NO: 432 - protein, 107 aa, synthetic construct
  REGION 1..107: note = Synthetic Construct
EIVMTQSPATLSVSPGERATLSCRASQSVDSNLVWYQQKPGQVPRLLIYGASTRATGIPARFSGSGSGTEFTLTISSLQSEDFAVYYCQQYNKWPLTFGGGTKVEIK

SEQ ID NO: 433 - protein, 122 aa, synthetic construct; identical to SEQ ID NO: 431
  REGION 1..122: note = Synthetic Construct

SEQ ID NO: 434 - protein, 108 aa, synthetic construct
  REGION 1..108: note = Synthetic Construct
EIVMTQSPATLSVSPGERATLSCRASQSVSSNLAWNQQKPGQAPRLLIYGASTRATGIPARFSGSGSGTEFTLTISSLQSEDFAVYYCQQYNNWPMYTFGQGTKLEIK

SEQ ID NO: 435 - protein, 120 aa, synthetic construct
  REGION 1..120: note = Synthetic Construct
QVQLVESGGGVVQPGRSLRLSCEASGFTFSSSGMHWVRQAPGKGLQWVAVISYDGNNKFYEDSVKGRLTISRDNSNNTLWLQMNSLRVEDTAVYYCAKSGGRVGAAFAYWGQGTLVTVSS

SEQ ID NO: 436 - protein, 112 aa, synthetic construct
  REGION 1..112: note = Synthetic Construct
DIVNTQSPLSLPVTPGEPASISCRSSQSLLYGNGYNYLDWYLQKPGHSPQLLIYLGSNRGSGVPDRFSGSGSGTDFTLKISRVEAEDVGIYYCMQTLQTPFTFGPGTKMYIK

SEQ ID NO: 437 - protein, 125 aa, synthetic construct
  REGION 1..125: note = Synthetic Construct
EVQLLESGGGLVQPGGSLRLSCAGSGITFSSYAMSWVRQAPGKGLEWVSAISGNGGTTNYADSVKGRFTISRDNSKNTLYLQMNSLRAEDTAVYYCAKERILTSSWTRYGIMDVWGQGTTVTVSS

SEQ ID NO: 438 - protein, 107 aa, synthetic construct
  REGION 1..107: note = Synthetic Construct
EIVMTQSPATLSMSPGERATLSCRASQSVSSNLAWYQQKPGQAPRLLIYGASTRATGIPARFSGSGSGTEFTLTISSLQSEDFAVYYCQQYNNWPLTFGGGTKLEIK

SEQ ID NO: 439 - protein, 119 aa, synthetic construct
  REGION 1..119: note = Synthetic Construct
QVQLQQWGAGLLKPSETLSLTCAVYGGSFSGYYWSWIRQPPGKGLEWIGEINHSGNTNYNPSLKSRVTISVDTSKNQFSLKLNSVTAADTAVYYCARREATVTPYFDYWGQGTLVTVSS

SEQ ID NO: 440 - protein, 107 aa, synthetic construct
  REGION 1..107: note = Synthetic Construct
DIQLTQSPSFLSASVGDRVTITCWASQGISSYLAWYQQKPGKAPKLLIYAASTLQSGVPSRFSGSGSGTEFTLTISSLQPEDFATYYCQQLNSYPLTFGGGTKVEIK

SEQ ID NO: 441 - protein, 118 aa, synthetic construct
  REGION 1..118: note = Synthetic Construct
QVQLVESGGGVVQPGRSLRLSCVVSGFNFSRNGIHWVRQAPGKGLEWVAVISYDGRNKFYVESVKGRFTISRDNSKNTLYLQMNSLRVEDTAVYYCAKSSIGGFFEYWGQGTLVTVSS

SEQ ID NO: 442 - protein, 112 aa, synthetic construct
  REGION 1..112: note = Synthetic Construct
DIVMTQSPLSLPVTPGEPASISCRSSQSLLHSNGYNYLDWYLQKPGQSPQLMIYLGSHRASGVPDRFSGSGSGTDFTLKISRVEAEDVGVYYCIQVQQTPITFGQGTRLEIK

SEQ ID NO: 443 - protein, 112 aa, synthetic construct
  REGION 1..112: note = Synthetic Construct
DIVMTQTPLSSPVTLGQPASISCRSSQSLVHSDGNTYLSWLQQRPGQPPRLLIYKISNRFSGVPDRFSGSGAGTDFTLKISRVEAEDVGVYFCMQATQFPYTFGQGTKLEIK

SEQ ID NO: 444 - protein, 116 aa, synthetic construct
  REGION 1..116: note = Synthetic Construct
EVQLLESGGGLVQPGGSLRLSCAASGFTFSSYAMSWVRQAPGKGLEWVSTISSGGSYTSYPDSVKGRFTISRDNSKNTLYLQMNSLRAEDTAVYYCAKQDYAMNYWGQGTMVTVSS

SEQ ID NO: 445 - protein, 107 aa, synthetic construct; identical to SEQ ID NO: 358
  REGION 1..107: note = Synthetic Construct

SEQ ID NO: 446 - protein, 116 aa, synthetic construct
  REGION 1..116: note = Synthetic Construct
EVQLVESGGGLVKPGGSLKLSCAASGFTFSSYAMSWVRQTPEKRLEWVATISSGGSYTSYPDSVKGRFTISRDNAKNTLYLQMSSLRSEDTAMYYCARQDYAMNYWGQGTSVTVSS

SEQ ID NO: 447 - protein, 107 aa, synthetic construct
  REGION 1..107: note = Synthetic Construct
DIEMTQSHKFMSTSVGDRVSITCKASQDVSTAVAWYQQKPGQSPKLLLYSASYRYTGVPDRFTGSGSGTDFTFTISSVQAEDLAVYYCQQHYSTPWTFGGGTKLEIK

SEQ ID NO: 448 - protein, 119 aa, synthetic construct
  REGION 1..119: note = Synthetic Construct
QVTLKESGPVLVKPTETLTLTCTVSGIDLSSYDISWVRQAPGKGLEWVGIISYAGSTYYASWAKGRLTISKDTSKNQVVLTMTNMDPVDTATYYCARGVPAYSTGGDLWGQGTLVTVSS

SEQ ID NO: 449 - protein, 119 aa, synthetic construct
  REGION 1..119: note = Synthetic Construct
QVTLKESGPVLVKPTETLTLTCTVSGIDLSHYDISWVRQAPGKGLEWVGIISYAGSTYYASWAKGRLTISKDTSKNQVVLTMTNMDPVDTATYYCARGVPAYSTGGDLWGQGTLVTVSS

SEQ ID NO: 450 - protein, 119 aa, synthetic construct
  REGION 1..119: note = Synthetic Construct
QVTLKESGPVLVKPTETLTLTCTVSGIDLSSYDISWVRQAPGKGLEWVGIISHAGSTYYASWAKGRLTISKDTSKNQVVLTMTNMDPVDTATYYCARGVPAYSTGGDLWGQGTLVTVSS

SEQ ID NO: 451 - protein, 119 aa, synthetic construct
  REGION 1..119: note = Synthetic Construct
QVTLKESGPVLVKPTETLTLTCTVSGIDLSSYDISWVRQAPGKGLEWVGIISYAGSTYYASWAKGRLTISKDTSKNQVVLTMTNMDPVDTATYYCARGVPAHSTGGDLWGQGTLVTVSS

SEQ ID NO: 452 - protein, 119 aa, synthetic construct
  REGION 1..119: note = Synthetic Construct
QVTLKESGPVLVKPTETLTLTCTVSGIDLSSYDISWVRQAPGKGLEWVGIISYAGSTYYASWAKGRLTISKDTSKNQVVLTMTNMDPVDTATYYCARGVPAYSHGGDLWGQGTLVTVSS

SEQ ID NO: 453 - protein, 119 aa, synthetic construct
  REGION 1..119: note = Synthetic Construct
QVTLKESGPVLVKPTETLTLTCTVSGIDLSHYDISWVRQAPGKGLEWVGIISHAGSTYYASWAKGRLTISKDTSKNQVVLTMTNMDPVDTATYYCARGVPAYSTGGDLWGQGTLVTVSS

SEQ ID NO: 454 - protein, 119 aa, synthetic construct
  REGION 1..119: note = Synthetic Construct
QVTLKESGPVLVKPTETLTLTCTVSGIDLSSYDISWVRQAPGKGLEWVGIISHAGSTYYASWAKGRLTISKDTSKNQVVLTMTNMDPVDTATYYCARGVPAHSTGGDLWGQGTLVTVSS

SEQ ID NO: 455 - protein, 119 aa, synthetic construct
  REGION 1..119: note = Synthetic Construct
QVTLKESGPVLVKPTETLTLTCTVSGIDLSHYDISWVRQAPGKGLEWVGIISYAGSTYYASWAKGRLTISKDTSKNQVVLTMTNMDPVDTATYYCARGVPAHSTGGDLWGQGTLVTVSS

SEQ ID NO: 456 - protein, 119 aa, synthetic construct
  REGION 1..119: note = Synthetic Construct
QVTLKESGPVLVKPTETLTLTCTVSGIDLSHYDISWVRQAPGKGLEWVGIISHAGSTYYASWAKGRLTISKDTSKNQVVLTMTNMDPVDTATYYCARGVPAHSTGGDLWGQGTLVTVSS

SEQ ID NO: 457 - protein, 119 aa, synthetic construct
  REGION 1..119: note = Synthetic Construct
QVTLKESGPVLVKPTETLTLTCTVSGIDLSHYDISWVRQAPGKGLEWVGIISYAGSTYYASWAKGRLTISKDTSKNQVVLTMTNMDPVDTATYYCARGVPAYSHGGDLWGQGTLVTVSS

SEQ ID NO: 458 - protein, 119 aa, synthetic construct
  REGION 1..119: note = Synthetic Construct
QVTLKESGPVLVKPTETLTLTCTVSGIDLSSYDISWVRQAPGKGLEWVGIISHAGSTYYASWAKGRLTISKDTSKNQVVLTMTNMDPVDTATYYCARGVPAYSHGGDLWGQGTLVTVSS

SEQ ID NO: 459 - protein, 119 aa, synthetic construct
  REGION 1..119: note = Synthetic Construct
QVTLKESGPVLVKPTETLTLTCTVSGIDLSSYDISWVRQAPGKGLEWVGIISYAGSTYYASWAKGRLTISKDTSKNQVVLTMTNMDPVDTATYYCARGVPAHSHGGDLWGQGTLVTVSS

SEQ ID NO: 460 - protein, 119 aa, synthetic construct
  REGION 1..119: note = Synthetic Construct
QVTLKESGPVLVKPTETLTLTCTVSGIDLSHYDISWVRQAPGKGLEWVGIISHAGSTYYASWAKGRLTISKDTSKNQVVLTMTNMDPVDTATYYCARGVPAYSHGGDLWGQGTLVTVSS

SEQ ID NO: 461 - protein, 119 aa, synthetic construct
  REGION 1..119: note = Synthetic Construct
QVTLKESGPVLVKPTETLTLTCTVSGIDLSSYDISWVRQAPGKGLEWVGIISHAGSTYYASWAKGRLTISKDTSKNQVVLTMTNMDPVDTATYYCARGVPAHSHGGDLWGQGTLVTVSS

SEQ ID NO: 462 - protein, 119 aa, synthetic construct
  REGION 1..119: note = Synthetic Construct
QVTLKESGPVLVKPTETLTLTCTVSGIDLSHYDISWVRQAPGKGLEWVGIISYAGSTYYASWAKGRLTISKDTSKNQVVLTMTNMDPVDTATYYCARGVPAHSHGGDLWGQGTLVTVSS

SEQ ID NO: 463 - protein, 119 aa, synthetic construct
  REGION 1..119: note = Synthetic Construct
QVTLKESGPVLVKPTETLTLTCTVSGIDLSHYDISWVRQAPGKGLEWVGIISHAGSTYYASWAKGRLTISKDTSKNQVVLTMTNMDPVDTATYYCARGVPAHSHGGDLWGQGTLVTVSS

SEQ ID NO: 464 - protein, 119 aa, synthetic construct
  REGION 1..119: note = Synthetic Construct
QVTLKESGPVLVKPTETLTLTCTVSGIDLSHTDISWVRQAPGKGLEWVGIISYAGSTYYASWAKGRLTISKDTSKNQVVLTMTNMDPVDTATYYCARGVPAYSHGGDLWGQGTLVTVSS

SEQ ID NO: 465 - protein, 119 aa, synthetic construct
  REGION 1..119: note = Synthetic Construct
QVTLKESGPVLVKPTETLTLTCTVSGIDLSHTDISWVRQAPGKGLEWVGIISYAGSTYYMSWAKGRLTISKDTSKNQVVLTMTNMDPVDTATYYCARGVPAYSHGGDLWGQGTLVTVSS

SEQ ID NO: 466 - protein, 119 aa, synthetic construct; identical to SEQ ID NO: 457
  REGION 1..119: note = Synthetic Construct

SEQ ID NO: 467 - protein, 119 aa, synthetic construct
  REGION 1..119: note = Synthetic Construct
QVTLKESGPVLVKPTETLTLTCTVSGIDLSHDDISWVRQAPGKGLEWVGIISYAGSTYYASWAKGRLTISKDTSKNQVVLTMTNMDPVDTATYYCARGVPAYSHGGDLWGQGTLVTVSS

SEQ ID NO: 468 - protein, 119 aa, synthetic construct
  REGION 1..119: note = Synthetic Construct
QVTLKESGPVLVKPTETLTLTCTVSGIDLSHDDISWVRQAPGKGLEWVGIISYAGSMYYASWAKGRLTISKDTSKNQVVLTMTNMDPVDTATYYCARGVPAYSHGGDKWGQGTLVTVSS

SEQ ID NO: 469 - protein, 119 aa, synthetic construct
  REGION 1..119: note = Synthetic Construct
QVTLKESGPVLVKPTETLTLTCTVSGIDLSHTDISWVRQAPGKGLEWVGIISYAGKTYYASWAKKRLTISKDTSKNQVVLTMTNMDPVDTATYYCARGVPAYSHGGDLWGQGTLVTVSS

SEQ ID NO: 470 - protein, 119 aa, synthetic construct
  REGION 1..119: note = Synthetic Construct
QVTLKESGPVLVKPTETLTLTCTVSGIDLSHTDISWVRQAPGKGLEWVGIISYAGKKYYASWAKGRLTISKDTSKNQVVLTMTNMDPVDTATYYCARGVPAYSHGGDLWGQGTLVTVSS

SEQ ID NO: 471 - protein, 119 aa, synthetic construct
  REGION 1..119: note = Synthetic Construct
QVTLKESGPVLVKPTETLTLTCTVSGIDLSHTDISWVRQAPGKGLEWVGIISYAGSKYKASWAKGRLTISKDTSKNQVVLTMTNMDPVDTATYYCARGVPAYSHGGDLWGQGTLVTVSS

SEQ ID NO: 472 - protein, 119 aa, synthetic construct
  REGION 1..119: note = Synthetic Construct
QVTLKESGPVLVKPTETLTLTCTVSGIDLSHDDISWVRQAPGKGLEWVGIISYAGKKYYASWAKGRLTISKDTSKNQVVLTMTNMDPVDTATYYCARGVPAYSHGGDLWGQGTLVTVSS

SEQ ID NO: 473 - protein, 119 aa, synthetic construct
  REGION 1..119: note = Synthetic Construct
QVQLVESGGGLVQPGGSLRLSCAVSGIDLSHDDISWVRQAPGKGLEWVSIISYAGSTYYASWAKGRLTISKDTSKNQVVLTMTNMDPVDTATYYCARGVPAYSHGGDLWGQGTLVTVSS

SEQ ID NO: 474 - protein, 119 aa, synthetic construct
  REGION 1..119: note = Synthetic Construct
QVTLKESGPVLVKPTETLTLTCKVSGIDLSHDDISWVRQAPGKGLEWVSIISYAGSTYYASWAKGRLTISKDTSKNQVVLTMTNMDPVDTATYYCARGVPAYSHGGDLWGQGTLVTVSS

SEQ ID NO: 475 - protein, 119 aa, synthetic construct
  REGION 1..119: note = Synthetic Construct
QVTLKESGPVLVKPTETLTLTCKVSGIDLSHEDISWVRQAPGKGLEWVSIISYAGSTYYASWAKGRLTISKDTSKNQVVLTMTNMDPVDTATYYCARGVPAYSHGGDLWGQGTLVTVSS

SEQ ID NO: 476 - protein, 119 aa, synthetic construct
  REGION 1..119: note = Synthetic Construct
QVQLVESGGGLVQPGGSLRLSCAVSGIDLSHEDISWVRQAPGKGLEWVSIISYAGSKYYASWAKGRLTISKDTSKNQVVLTMTNMDPVDTATYYCARGVPAYSHGGDLWGQGTLVTVSS

SEQ ID NO: 477 - protein, 110 aa, synthetic construct
  REGION 1..110: note = Synthetic Construct
DIVMTQSPATLSLSPGERATLSCQSSQSVYDNNWLSWFQQKPGQPPKLLIYWASTLASGVPSRFSGSGSGTDFTLTISSLQPEDAATYYCAGGYGGGLYAFGQGTKVEIK

SEQ ID NO: 478 - protein, 110 aa, synthetic construct
  REGION 1..110: note = Synthetic Construct
DIVMTQSPATLSLSPGERATLSCQSSQSVYHNNWLSWFQQKPGQPPKLLIYWASTLASGVPSRFSGSGSGTDFTLTISSLQPEDAATYYCAGGYGGGLYAFGQGTKVEIK

SEQ ID NO: 479 - protein, 110 aa, synthetic construct
  REGION 1..110: note = Synthetic Construct
DIVMTQSPATLSLSPGERATLSCTSSQSVYHNNWLSWFQQKPGQPPKLLIYWASTLAYGVPSRFSGSGSGTDFTLTISSLQPEDAATYYCAGGYGGGRYAFGQGTKVEIK

SEQ ID NO: 480 - protein, 110 aa, synthetic construct
  REGION 1..110: note = Synthetic Construct
DIVMTQSPATLSLSPGERATLSCQSSQSVYHNNWLSWFQQKPGQPPKLLIYWASTLAFGVPSRFSGSGSGTDFTLTISSLQPEDAATYYCAGGYGGGLYAFGQGTKVEIK

SEQ ID NO: 481 - protein, 110 aa, synthetic construct
  REGION 1..110: note = Synthetic Construct
DIVMTQSPATLSLSPGERATLSCQTSQSVYHDNWLSWFQQKPGQPPKLLIYWASTLASGVPSRFSGSGSGTDFTLTISSLQPEDAATYYCAGGYGGGRYAFGQGTKVEIK

SEQ ID NO: 482 - protein, 110 aa, synthetic construct
  REGION 1..110: note = Synthetic Construct
DIVMTQSPATLSLSPGERATLSCTSSQSVYHDNWLSWFQQKPGQPPKLLIYWASTLAYGVPSRFSGSGSGTDFTLTISSLQPEDAATYYCAGGYGGGLYAFGQGTKVEIK

SEQ ID NO: 483 - protein, 110 aa, synthetic construct
  REGION 1..110: note = Synthetic Construct
DIVMTQSPATLSLSPGERATLSCTSSQSVYHANWLSWFQQKPGQPPKLLIYWASTLAYGVPSRFSGSGSGTDFTLTISSLQPEDAATYYCAGGYGGGRYAFGQGTKVEIK

SEQ ID NO: 484 - protein, 110 aa, synthetic construct
  REGION 1..110: note = Synthetic Construct
DIVMTQSPATLSLSPGERATLSCTTSQSVYHENWLSWFQQKPGQPPKLLIYWASTLAYGVPSRFSGSGSGTDFTLTISSLQPEDAATYYCAGGYGGGRYAFGQGTKVEIK

SEQ ID NO: 485 - protein, 110 aa, synthetic construct
  REGION 1..110: note = Synthetic Construct
DIVMTQSPATLSLSPGERATLSCTSSQSVFHENWLSWFQQKPGQPPKLLIYWASTLAWGVPSRFSGSGSGTDFTLTISSLQPEDAATYYCAGGYGGGRYAFGQGTKVEIK

SEQ ID NO: 486 - protein, 110 aa, synthetic construct
  REGION 1..110: note = Synthetic Construct
DIVMTQSPATLSLSPGERATLSCTTSQSVYHENWLSWYQQKPGQPPKLLIYWASTLAYGVPSRFSGSGSGTDFTLTISSLQPEDVATYYCAGGYGGGRYAFGQGTKVEIK

SEQ ID NO: 487 - protein, 5 aa, synthetic construct
  REGION 1..5: note = Synthetic Construct
HDDIS

SEQ ID NO: 488 - protein, 16 aa, synthetic construct
  REGION 1..16: note = Synthetic Construct
IISYAGSTYYASWAKG

SEQ ID NO: 489 - protein, 11 aa, synthetic construct
  REGION 1..11: note = Synthetic Construct
GVPAYSHGGDL

SEQ ID NO: 490 - protein, 13 aa, synthetic construct
  REGION 1..13: note = Synthetic Construct
TSSQSVYHANWLS

SEQ ID NO: 491 - protein, 7 aa, synthetic construct
  REGION 1..7: note = Synthetic Construct
WASTLAY

SEQ ID NO: 492 - protein, 10 aa, synthetic construct
  REGION 1..10: note = Synthetic Construct
AGGYGGGRYA

SEQ ID NO: 493 - protein, 13 aa, synthetic construct
  REGION 1..13: note = Synthetic Construct
TTSQSVYHENWLS

SEQ ID NO: 494 - protein, 157 aa, synthetic construct
  REGION 1..157: note = Synthetic Construct
MKCSWIMFFLVATATGVHSQVQLQQPGAELVKPGASVKLSCKASGYTFTNYWMYWVKQRPGQGLEWIGMIHPNSGSTNYNGKFKTGATLTVDKSSSTVYMQLSSLTSEDSAVYYCARWGYGGNYDYAMDYWGQGTSVTVSSAKTTPPSVYPLAPGSL

SEQ ID NO: 495 - protein, 165 aa, synthetic construct
  REGION 1..165: note = Synthetic Construct
MDFQVQIFSFLLISASVIMSRGQIVLTQSPAIMSASLGERVTMTCTASSSVSSSYFHWYQQKPGSSPKLWIYSTSNLASGVPARFSGSGSGTSYSLTISTMEAEDAVTYYCHQYHRSPWTFGGGTKLEIKRADAAPTVSIFPPSSEQLTSGGASVVCFLNNFYPK

SEQ ID NO: 496 - protein, 5 aa, synthetic construct
  REGION 1..5: note = Synthetic Construct
NSAMS

SEQ ID NO: 497 - protein, 18 aa, synthetic construct
  REGION 1..18: note = Synthetic Construct
GISVTGGSTFYTDSVKGR

SEQ ID NO: 498 - protein, 9 aa, synthetic construct
  REGION 1..9: note = Synthetic Construct
VYYYSFFDY

SEQ ID NO: 499 - protein, 14 aa, synthetic construct
  REGION 1..14: note = Synthetic Construct
GFNSGSVSTSYWPS

SEQ ID NO: 500 - protein, 7 aa, synthetic construct
  REGION 1..7: note = Synthetic Construct
NTNTRSS

SEQ ID NO: 501 - protein, 10 aa, synthetic construct
  REGION 1..10: note = Synthetic Construct
VLWMGSGIWV

SEQ ID NO: 502 - protein, 5 aa, synthetic construct
  REGION 1..5: note = Synthetic Construct
NYAMS

SEQ ID NO: 503 - protein, 18 aa, synthetic construct
  REGION 1..18: note = Synthetic Construct
GISVTGGSTYYTDSVKGR

SEQ ID NO: 504 - protein, 9 aa, synthetic construct
  REGION 1..9: note = Synthetic Construct
VYYSSFFDY

SEQ ID NO: 505 - protein, 14 aa, synthetic construct
  REGION 1..14: note = Synthetic Construct
GFNSGSVSTSYYPS

SEQ ID NO: 506 - protein, 7 aa, synthetic construct
  REGION 1..7: note = Synthetic Construct
NTNTRSS

SEQ ID NO: 507 - protein, 10 aa, synthetic construct
  REGION 1..10: note = Synthetic Construct
VLYMGSGIWV

SEQ ID NO: 508 - protein, 444 aa, synthetic construct
  REGION 1..444: note = Synthetic Construct
EVQLLESGGGLVQPGGSLRLSCAASGFTFRNSAMSWVRQAPGKGLEWVSGISVTGGSTFYTDSVKGRFTVSRDNSRNTLYLQMNSLRAEDTAVYYCAKVYYYSFFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 509 - protein, 216 aa, synthetic construct
  REGION 1..216: note = Synthetic Construct
QTVVTQEPSFSVSPGGTVTLTCGFNSGSVSTSYWPSWYQQTPGQAPRTLIYNTNTRSSGVPDRFSGSILGNKAALTITGAQADDESDYYCVLWMGSGIWVFGGGTKLTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 510 - protein, 444 aa, synthetic construct
  REGION 1..444: note = Synthetic Construct
EVQLLESGGGLVQPGGSLRLSCAASGFTFRNDAMSWVRQAPGKGLEWVSGISVTGGSTFYTDSVKGRFTVSRDNSRNTLYLQMNSLRAEDTAVYYCAKVYYTSFFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 511 - protein, 216 aa, synthetic construct
  REGION 1..216: note = Synthetic Construct
QTVVTQEPSFSVSPGGTVTLTCGFNSGSVSTSYYPSWYQQTPGQAPRTLIYNTNTRSSGVPDRFSGSILGNKAALTITGAQADDESDYYCVLYMGSGIWVFGGGTKLTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 512 - protein, 115 aa, synthetic construct
  REGION 1..115: note = Synthetic Construct
QVQLVQSGAEVKKPGASVKVSCKASGYTFTSSYINWVRQAPGQGLEWMGTINPVSGSTSYAQKFQGRVTMTRDTSISTAYMELSSLRSEDTAVYYCARGGWFDYWGQGTLVTVSS

SEQ ID NO: 513 - protein, 113 aa, synthetic construct
  REGION 1..113: note = Synthetic Construct
DIALTQPASVSGSPGQSITISCTGTSSDVGSYNYVNWYQQHPGKAPKLMIYGVSKRPSGVSNRFSGSKSGNTASLTISGLQAEDEADYYCGTFAGGSYYGVFGGGTKLTVLGQ

SEQ ID NO: 514 - protein, 115 aa, synthetic construct
  REGION 1..115: note = Synthetic Construct
QVQLVQSGAEVKKPGASVKVSCKASGYTFTSSYINWVRQAPGQGLEWMGTINPVSGNTSYAQKFQGRVTMTRDTSISTAYMELSSLRSEDTAVYYCARGGWFDYWGQGTLVTVSS

SEQ ID NO: 515 - protein, 112 aa, synthetic construct
  REGION 1..112: note = Synthetic Construct
DIALTQPASVSGSPGQSITISCTGTSSDVGSYNYVNWYQQHPGKAPKLMIYGVSKRPSGVSNRFSGSKSGNTASLTISGLQAEDEADYYCQAWTSKMAGVFGGGTKLTVLGQ

SEQ ID NO: 516 - protein, 115 aa, synthetic construct; identical to SEQ ID NO: 514
  REGION 1..115: note = Synthetic Construct

SEQ ID NO: 517 - protein, 112 aa, synthetic construct
  REGION 1..112: note = Synthetic Construct
DIALTQPASVSGSPGQSITISCTGTSSDVGSYNYVNWYQQHPGKAPKLMIYGVSKRPSGVSNRFSGSKSGNTASLTISGLQAEDEADYYCSSYTRMGHPVFGGGTKLTVLGQ

SEQ ID NO: 518 - protein, 115 aa, synthetic construct; identical to SEQ ID NO: 514
  REGION 1..115: note = Synthetic Construct

SEQ ID NO: 519 - protein, 113 aa, synthetic construct
  REGION 1..113: note = Synthetic Construct
DIALTQPASVSGSPGQSITISCTGTSSDVGSYNYVNWYQQHPGKAPKLMIYGVSKRPSGVSNRFSGSKSGNTASLTISGLQAEDEADYYCATYGKGVTPPVFGGGTKLTVLGQ

SEQ ID NO: 520 - protein, 115 aa, synthetic construct; identical to SEQ ID NO: 514
  REGION 1..115: note = Synthetic Construct

SEQ ID NO: 521 - protein, 113 aa, synthetic construct; identical to SEQ ID NO: 513
  REGION 1..113: note = Synthetic Construct

SEQ ID NO: 522 - protein, 115 aa, synthetic construct
  REGION 1..115: note = Synthetic Construct
QVQLVQSGAEVKKPGASVKVSCKASGYTFTSSYINWVRQAPGQGLEWMGMINAPIGTTRYAQKFQGRVTMTRDTSISTAYMELSSLRSEDTAVYYCARGGWFDYWGQGTLVTVSS

SEQ ID NO: 523 - protein, 112 aa, synthetic construct; identical to SEQ ID NO: 515
  REGION 1..112: note = Synthetic Construct

SEQ ID NO: 524 - protein, 115 aa, synthetic construct
  REGION 1..115: note = Synthetic Construct
QVQLVQSGAEVKKPGASVKVSCKASGYTFTSSYINWVRQAPGQGLEWMGQINAASGMTRYAQKFQGRVTMTRDTSISTAYMELSSLRSEDTAVYYCARGGWFDYWGQGTLVTVSS

SEQ ID NO: 525 - protein, 112 aa, synthetic construct; identical to SEQ ID NO: 515
  REGION 1..112: note = Synthetic Construct

SEQ ID NO: 526 - protein, 115 aa, synthetic construct; identical to SEQ ID NO: 522
  REGION 1..115: note = Synthetic Construct

SEQ ID NO: 527 - protein, 113 aa, synthetic construct; identical to SEQ ID NO: 513
  REGION 1..113: note = Synthetic Construct

SEQ ID NO: 528 - protein, 115 aa, synthetic construct
  REGION 1..115: note = Synthetic Construct
QVQLVQSGAEVKKPGASVKVSCKASGYTFTSSYINWVRQAPGQGLEWMGTINPVSGNTRYAQKFQGRVTMTRDTSISTAYMELSSLRSEDTAVYYCARGGWFDYWGQGTLVTVSS

SEQ ID NO: 529 - protein, 113 aa, synthetic construct; identical to SEQ ID NO: 513
  REGION 1..113: note = Synthetic Construct

SEQ ID NO: 530 - protein, 115 aa, synthetic construct; identical to SEQ ID NO: 524
  REGION 1..115: note = Synthetic Construct

SEQ ID NO: 531 - protein, 113 aa, synthetic construct; identical to SEQ ID NO: 513
  REGION 1..113: note = Synthetic Construct

SEQ ID NO: 532 - protein, 115 aa, synthetic construct
  REGION 1..115: note = Synthetic Construct
QVQLVQSGAEVKKPGASVKVSCKASGYTFTSSYINWVRQAPGQGLEWMGNINAAAGITLYAQKFQGRVTMTRDTSISTAYMELSSLRSEDTAVYYCARGGWFDYWGQGTLVTVSS

SEQ ID NO: 533 - protein, 113 aa, synthetic construct; identical to SEQ ID NO: 513
  REGION 1..113: note = Synthetic Construct

SEQ ID NO: 534 - protein, 115 aa, synthetic construct
  REGION 1..115: note = Synthetic Construct
QVQLVQSGAEVKKPGASVKVSCKASGYTFTSSYINWVRQAPGQGLEWMGTINPPTGGTYYAQKFQGRVTMTRDTSISTAYMELSSLRSEDTAVYYCARGGWFDYWGQGTLVTVSS

SEQ ID NO: 535 - protein, 113 aa, synthetic construct; identical to SEQ ID NO: 513
  REGION 1..113: note = Synthetic Construct

SEQ ID NO: 536 - protein, 115 aa, synthetic construct
  REGION 1..115: note = Synthetic Construct
QVQLVQSGAEVKKPGASVKVSCKASGYTFTSSYINWVRQAPGQGLEWMGGINPPAGTTSYAQKFQGRVTMTRDTSISTAYMELSSLRSEDTAVYYCARGGWFDYWGQGTLVTVSS

SEQ ID NO: 537 - protein, 113 aa, synthetic construct; identical to SEQ ID NO: 513
  REGION 1..113: note = Synthetic Construct

SEQ ID NO: 538 - protein, 115 aa, synthetic construct
  REGION 1..115: note = Synthetic Construct
QVQLVQSGAEVKKPGASVKVSCKASGYTFTSSYINWVRQAPGQGLEWMGNINPATGHADYAQKFQGRVTMTRDTSISTAYMELSSLRSEDTAVYYCARGGWFDYWGQGTLVTVSS

SEQ ID NO: 539 - protein, 113 aa, synthetic construct; identical to SEQ ID NO: 513
  REGION 1..113: note = Synthetic Construct

SEQ ID NO: 540 - protein, 10 aa, synthetic construct
  REGION 1..10: note = Synthetic Construct
GYTFTSSYIN

SEQ ID NO: 541 - protein, 17 aa, synthetic construct
  REGION 1..17: note = Synthetic Construct
TINPVSGSTSYAQKFQG

SEQ ID NO: 542 - protein, 6 aa, synthetic construct
  REGION 1..6: note = Synthetic Construct
GGWFDY

SEQ ID NO: 543 - protein, 14 aa, synthetic construct
  REGION 1..14: note = Synthetic Construct
TGTSSDVGSYNYVN

SEQ ID NO: 544 - protein, 11 aa, synthetic construct
  REGION 1..11: note = Synthetic Construct
LMIYGVSKRPS

SEQ ID NO: 545 - protein, 10 aa, synthetic construct
  REGION 1..10: note = Synthetic Construct
GTFAGGSYYG

SEQ ID NO: 546 - protein, 17 aa, synthetic construct
  REGION 1..17: note = Synthetic Construct
TINPVSGNTSYAQKFQG

SEQ ID NO: 547 - protein, 9 aa, synthetic construct
  REGION 1..9: note = Synthetic Construct
QAWTSKMAG

SEQ ID NO: 548 - protein, 9 aa, synthetic construct
  REGION 1..9: note = Synthetic Construct
SSYTRMGHP

SEQ ID NO: 549 - protein, 10 aa, synthetic construct
  REGION 1..10: note = Synthetic Construct
ATYGKGVTPP

SEQ ID NO: 550 - protein, 17 aa, synthetic construct
  REGION 1..17: note = Synthetic Construct
MINAPIGTTRYAQKFQG

SEQ ID NO: 551 - protein, 17 aa, synthetic construct
  REGION 1..17: note = Synthetic Construct
QINAASGMTRYAQKFQG

SEQ ID NO: 552 - protein, 17 aa, synthetic construct
  REGION 1..17: note = Synthetic Construct
TINPVSGNTRYAQKFQG

SEQ ID NO: 553 - protein, 17 aa, synthetic construct
  REGION 1..17: note = Synthetic Construct
NINAAAGITLYAQKFQG

SEQ ID NO: 554 - protein, 17 aa, synthetic construct
  REGION 1..17: note = Synthetic Construct
TINPPTGGTYYAQKFQG

SEQ ID NO: 555 - protein, 17 aa, synthetic construct
  REGION 1..17: note = Synthetic Construct
GINPPAGTTSYAQKFQG

SEQ ID NO: 556 - protein, 17 aa, synthetic construct
  REGION 1..17: note = Synthetic Construct
NINPATGHADYAQKFQG

SEQ ID NO: 557 - protein, 445 aa, synthetic construct
  REGION 1..445: note = Synthetic Construct
QVQLVQSGAEVKKPGASVKVSCKASGYTFTSSYINWVRQAPGQGLEWMGTINPVSGSTSYAQKFQGRVTMTRDTSISTAYMELSRLRSDDTAVYYCARGGWFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 558 - protein, 217 aa, synthetic construct
  REGION 1..217: note = Synthetic Construct
QSALTQPASVSGSPGQSITISCTGTSSDVGSYNYVNWYQQHPGKAPKLMIYGVSKRPSGVSNRFSGSKSGNTASLTISGLQAEDEADYYCGTFAGGSYYGVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 559 - protein, 445 aa, synthetic construct
  REGION 1..445: note = Synthetic Construct
QVQLVQSGAEVKKPGASVKVSCKASGYTFTSSYINWVRQAPGQGLEWMGQINAASGMTRYAQKFQGRVTMTRDTSISTAYMELSRLRSDDTAVYYCARGGWFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 560 - protein, 217 aa, synthetic construct; identical to SEQ ID NO: 558
  REGION 1..217: note = Synthetic Construct

SEQ ID NO: 561 - protein, 445 aa, synthetic construct
  REGION 1..445: note = Synthetic Construct
QVQLVQSGAEVKKPGASVKVSCKASGYTFTSSYINWVRQAPGQGLEWMGNINAAAGITLYAQKFQGRVTMTRDTSISTAYMELSRLRSDDTAVYYCARGGWFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 562 - protein, 217 aa, synthetic construct; identical to SEQ ID NO: 558
  REGION 1..217: note = Synthetic Construct

SEQ ID NO: 563 - protein, 445 aa, synthetic construct
  REGION 1..445: note = Synthetic Construct
QVQLVQSGAEVKKPGASVKVSCKASGYTFTSSYINWVRQAPGQGLEWMGGINPPAGTTSYAQKFQGRVTMTRDTSISTAYMELSRLRSDDTAVYYCARGGWFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 564 - protein, 217 aa, synthetic construct; identical to SEQ ID NO: 558
  REGION 1..217: note = Synthetic Construct

SEQ ID NO: 565 - protein, 445 aa, synthetic construct
  REGION 1..445: note = Synthetic Construct
QVQLVQSGAEVKKPGASVKVSCKASGYTFTSSYINWVRQAPGQGLEWMGNINPATGHADYAQKFQGRVTMTRDTSISTAYMELSRLRSDDTAVYYCARGGWFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 566 - protein, 217 aa, synthetic construct; identical to SEQ ID NO: 558
  REGION 1..217: note = Synthetic Construct

SEQ ID NO: 567 - protein, 441 aa, synthetic construct
  REGION 1..441: note = Synthetic Construct
QVQLVQSGAEVKKPGASVKVSCKASGYTFTSSYINWVRQAPGQGLEWMGTINPVSGSTSYAQKFQGRVTMTRDTSISTAYMELSRLRSDDTAVYYCARGGWFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 568 - protein, 217 aa, synthetic construct; identical to SEQ ID NO: 558
  REGION 1..217: note = Synthetic Construct

SEQ ID NO: 569 - protein, 441 aa, synthetic construct
  REGION 1..441: note = Synthetic Construct
QVQLVQSGAEVKKPGASVKVSCKASGYTFTSSYINWVRQAPGQGLEWMGQINAASGMTRYAQKFQGRVTMTRDTSISTAYMELSRLRSDDTAVYYCARGGWFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 570 - protein, 217 aa, synthetic construct; identical to SEQ ID NO: 558
  REGION 1..217: note = Synthetic Construct

SEQ ID NO: 571 - protein, 441 aa, synthetic construct
  REGION 1..441: note = Synthetic Construct
QVQLVQSGAEVKKPGASVKVSCKASGYTFTSSYINWVRQAPGQGLEWMGNINAAAGITLYAQKFQGRVTMTRDTSISTAYMELSRLRSDDTAVYYCARGGWFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 572 - protein, 217 aa, synthetic construct; identical to SEQ ID NO: 558
  REGION 1..217: note = Synthetic Construct

SEQ ID NO: 573 - protein, 441 aa, synthetic construct
  REGION 1..441: note = Synthetic Construct
QVQLVQSGAEVKKPGASVKVSCKASGYTFTSSYINWVRQAPGQGLEWMGGINPPAGTTSYAQKFQGRVTMTRDTSISTAYMELSRLRSDDTAVYYCARGGWFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 574 - protein, 217 aa, synthetic construct; identical to SEQ ID NO: 558
  REGION 1..217: note = Synthetic Construct

SEQ ID NO: 575 - protein, 441 aa, synthetic construct
  REGION 1..441: note = Synthetic Construct
QVQLVQSGAEVKKPGASVKVSCKASGYTFTSSYINWVRQAPGQGLEWMGNINPATGHADYAQKFQGRVTMTRDTSISTAYMELSRLRSDDTAVYYCARGGWFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 576 - protein, 217 aa, synthetic construct; identical to SEQ ID NO: 558
  REGION 1..217: note = Synthetic Construct

SEQ ID NO: 577 - protein, 5 aa, synthetic construct
  REGION 1..5: note = Synthetic Construct
NYWMG

SEQ ID NO: 578 - protein, 17 aa, synthetic construct
  REGION 1..17: note = Synthetic Construct
YIRSSGGLTHYADSVKG

SEQ ID NO: 579 - protein, 7 aa, synthetic construct
  REGION 1..7: note = Synthetic Construct
GLYSFDY

SEQ ID NO: 580 - protein, 11 aa, synthetic construct
  REGION 1..11: note = Synthetic Construct
RASQGVNNFLA

SEQ ID NO: 581 - protein, 7 aa, synthetic construct
  REGION 1..7: note = Synthetic Construct
AASTLQS

SEQ ID NO: 582 - protein, 9 aa, synthetic construct
  REGION 1..9: note = Synthetic Construct
QQSYSTPRG

SEQ ID NO: 583 - protein, 120 aa, synthetic construct
  REGION 1..120: note = Synthetic Construct
DVQLQESGPGLVKPSQSLSLTCSVTGYSITSGYYWNWIRQFPGNKLEWMGYISYDGSNNYNPSLINRISITRDTSKNQFFLKLNSVTTEDTATYFCASYAYRNDVRFAYWGQGTLVTVSA

SEQ ID NO: 584 - protein, 107 aa, synthetic construct
  REGION 1..107: note = Synthetic Construct
DIQMTQTTSSLSASLGDRVTISCRASQDISNFLNWYQQKPDGTVKLLIYFTSRLHSGVPSRFSGSGSGTDYSLTITNLEQEDIATYFCQQGNTLPWTFGGGTKLEIK

SEQ ID NO: 585 - protein, 10 aa, synthetic construct
  REGION 1..10: note = Synthetic Construct
YSITSGYYWN

SEQ ID NO: 586 - protein, 16 aa, synthetic construct
  REGION 1..16: note = Synthetic Construct
YISYDGSNNYNPSLIN

SEQ ID NO: 587 - protein, 11 aa, synthetic construct
  REGION 1..11: note = Synthetic Construct
YAYRNDVRFAY

SEQ ID NO: 588 - protein, 11 aa, synthetic construct
  REGION 1..11: note = Synthetic Construct
RASQDISNFLN

SEQ ID NO: 589 - protein, 7 aa, synthetic construct
  REGION 1..7: note = Synthetic Construct
FTSRLHS

SEQ ID NO: 590 - protein, 9 aa, synthetic construct
  REGION 1..9: note = Synthetic Construct
QQGNTLPWT

SEQ ID NO: 591 - protein, 120 aa, synthetic construct
  REGION 1..120: note = Synthetic Construct
QVQLVQSGAEVKKPGASVKVSCKASGYTFTSYRMHWVRQAPGQGLEWMGFIVPSGGSTGYAQKFQGRVTMTRDTSTSTVYMELSSLRSEDTAVYYCARVSRYAPEPMDVWGQGTTVTVSS

SEQ ID NO: 592 - protein, 107 aa, synthetic construct
  REGION 1..107: note = Synthetic Construct
DIQMTQSPSSVSASVGDRVTITCRASQGISRWLAWYQQKPGKAPKLLIYAASSLQSGVPSRFSGSGSGTDFTLTISSLQPEDFATYYCQQAFSHPWTFGGGTKVEIK

SEQ ID NO: 593 - protein, 130 aa, synthetic construct
  REGION 1..130: note = Synthetic Construct
QLQLQESGPGLVKPSETLSLTCTVSGGSISSSSYAWGWIRQPPGKGLEWIGSIYYSGSTYYNPSLKSRVTISVDTSKNQFSLKLSSVTAADTAVYYCARDSGIGYSYASSHGYYYYMDVWGKGTTVTVSS

SEQ ID NO: 594 - protein, 107 aa, synthetic construct
  REGION 1..107: note = Synthetic Construct
EIVLTQSPATLSVSPGERATLSCRASQSVGSNLAWYQQKPGQAPRLLIYGASTRATGIPARFSGSGSGTEFTLTISSLQSEDFAVYYCQQYFHFPLTFGGGTKVEIK

SEQ ID NO: 595 - protein, 130 aa, synthetic construct
  REGION 1..130: note = Synthetic Construct
QVQLQESGPGLVKPSQTLSLTCTVSGGSIGSGGYYWSWIRQHPGKGLEWIGGIYGSGSTYYNPSLKSRVTISVDTSKNQFSLKLSSVTAADTAVYYCARDSGIGYSYASSHGYYYYMDVWGKGTTVTVSS

SEQ ID NO: 596 - protein, 130 aa, synthetic construct
  REGION 1..130: note = Synthetic Construct
QVQLQESGPGLVKPSQTLSLTCTVSGGSIKSGGYYWSWIRQHPGKGLEWIGGIYPSGSTYYNPSLKSRVTISVDTSKNQFSLKLSSVTAADTAVYYCARDSGIGYSYASSHGYYYYMDVWGKGTTVTVSS

SEQ ID NO: 597 - protein, 130 aa, synthetic construct
  REGION 1..130: note = Synthetic Construct
QVQLQESGPGLVKPSQTLSLTCTVSGGSILSGGYYWSWIRQHPGKGLEWIGGIYYSGKTYYNPSLKSRVTISVDTSKNQFSLKLSSVTAADTAVYYCARDSGIGYSYASSHGYYYYMDVWGKGTTVTVSS

SEQ ID NO: 598 - protein, 130 aa, synthetic construct
  REGION 1..130: note = Synthetic Construct
QVQLQESGPGLVKPSQTLSLTCTVSGGSISSGGYFWSWIRQHPGKGLEWIGGIYYSGRTYYNPSLKSRVTISVDTSKNQFSLKLSSVTAADTAVYYCARDSGIGYSYASSHGYYYYMDVWGKGTTVTVSS

SEQ ID NO: 599 - protein, 130 aa, synthetic construct
  REGION 1..130: note = Synthetic Construct
QVQLQESGPGLVKPSQTLSLTCTVSGGSIESGGYYWSWIRQHPGKGLEWIGGIYGSGSTYYNPSLKSRVTISVDTSKNQFSLKLSSVTAADTAVYYCARDSGIGYSYASSHGYYYYMDVWGKGTTVTVSS

SEQ ID NO: 600 - protein, 121 aa, synthetic construct
  REGION 1..121: note = Synthetic Construct
QLQLQESGPGLVKPSETLSLTCTVSGGSISSSSYAWGWIRQPPGKGLEWIGSIYYSGSTYYNPSLKSRVTISVDTSKNQFSLKLSSVTAADTAVYYCARAGKYRWHGMDVWGQGTTVTVSS

SEQ ID NO: 601 - protein, 107 aa, synthetic construct
  REGION 1..107: note = Synthetic Construct
DIQMTQSPSSVSASVGDRVTITCRASQGISSWLAWYQQKPGKAPKLLIYAASNLQSGVPSRFSGSGSGTDFTLTISSLQPEDFATYYCQQAPDLPITFGGGTKVEIK

SEQ ID NO: 602 - protein, 120 aa, synthetic construct
  REGION 1..120: note = Synthetic Construct
QVQLQESGPGLVKPSETLSLTCAVSGYSISSGVYWMWIRQPPGKGLEWIGSIVHSGHTYYNPSLKSRVTISVDTSKNQFSLKLSSVTAADTAVYYCARAGKYRWHGMDVWGQGTTVTVSS

SEQ ID NO: 603 - protein, 121 aa, synthetic construct
  REGION 1..121: note = Synthetic Construct
EVQLLESGGGLVQPGGSLRLSCAASGFTFSSYAMSWVRQAPGKGLEWVSGISGSGGSTYYADSVKGRFTISRDNSKNTLYLQMNSLRAEDTAVYYCAKDPLSLLLGYFDYWGQGALVTVSS

SEQ ID NO: 604 - protein, 107 aa, synthetic construct
  REGION 1..107: note = Synthetic Construct
DIQMTQSPSTLSASVGDRVTITCRASQSISSWLAWYQQKPGKAPKLLIYDASSLESGVPSRFSGSGSGTEFTLTISSLQPDDFATYYCQQYNRHSPTFGGGTKVEIK

SEQ ID NO: 605 - protein, 121 aa, synthetic construct
  REGION 1..121: note = Synthetic Construct
EVQLLESGGGLVQPGGSLRLSCAASGFTFSRYAMSWVRQAPGKGLEWVSGISGSGGATYYADSVKGRFTISRDNSKNTLYLQMNSLRAEDTAVYYCAKDPLSLLLGYFDYWGQGALVTVSS

SEQ ID NO: 606 - protein, 117 aa, synthetic construct
  REGION 1..117: note = Synthetic Construct
EVQLLESGGGLVQPGGSLRLSCAASGFTFGSYGMTWVRQAPGKGLEWVSVISGSGGGTYYADSVKGRFTISRDNSKNTLYLQMNSLRAEDTAVYYCAKGPRIVGMDVWGQGTTVTVS

SEQ ID NO: 607 - protein, 107 aa, synthetic construct
  REGION 1..107: note = Synthetic Construct
DIQMTQSPSSVSASVGDRVTITCRASQGISSWLAWYQQKPGKAPKLLIYAASSLQSGVPSRFSGSGSGTDFTLTISSLQPEDFATYYCQQVFSYPLTFGGGTKVEIK

SEQ ID NO: 608 - protein, 120 aa, synthetic construct
  REGION 1..120: note = Synthetic Construct
QVQLVQSGAEVKKPGASVKVSCKASGYTFTSYGISWVRQAPGQGLEWMGWISPYNGNTNYAQKLQGRVTMTTDTSTSTAYMELRSLRSDDTAVYYCARVSMYAPEPMDVWGQGTTVTVSS

SEQ ID NO: 609 - protein, 107 aa, synthetic construct; identical to SEQ ID NO: 592
  REGION 1..107: note = Synthetic Construct

SEQ ID NO: 610 - protein, 120 aa, synthetic construct
  REGION 1..120: note = Synthetic Construct
QVQLVQSGAEVKKPGASVKVSCKASGYTFTGHKMHWVRQAPGQGLEWMGWINPASGWTNYAQKFQGRVTMTRDTSISTAYMELSRLRSDDTAVYYCARVSMYAPEPMDVWGQGTTVTVSS

SEQ ID NO: 611 - protein, 120 aa, synthetic construct
  REGION 1..120: note = Synthetic Construct
QVQLVQSGAEVKKPGASVKVSCKASGYTFTSYNMAWVRQAPGQGLEWMGIIRPSVGSTSYAQKFQGRVTMTRDTSTSTVYMELSSLRSEDTAVYYCARVSMYAPEPMDVWGQGTTVTVSS

SEQ ID NO: 612 - protein, 120 aa, synthetic construct
  REGION 1..120: note = Synthetic Construct
QVQLVQSGAEVKKPGASVKVSCKASGYTFTSYRMHWVRQAPGQGLEWMGFIVPSGGSTSYAQKFQGRVTMTRDTSTSTVYMELSSLRSEDTAVYYCARVSMYAPEPMDVWGQGTTVTVSS

SEQ ID NO: 613 - protein, 120 aa, synthetic construct; identical to SEQ ID NO: 591
  REGION 1..120: note = Synthetic Construct

SEQ ID NO: 614 - protein, 122 aa, synthetic construct
  REGION 1..122: note = Synthetic Construct
QVQLQESGPGLVKPSQTLSLTCTVSGGSISSGSYYWSWIRQHPGKGLEWIGYIYYSGSTYYNPSLKSRVTISVDTSKNQFSLKLSSVTAADTAVYYCARGLGMYYHVPFDIWGQGTMVTVSS

SEQ ID NO: 615 - protein, 107 aa, synthetic construct
  REGION 1..107: note = Synthetic Construct
EIVLTQSPATLSLSPGERATLSCRASQSVSSYLAWYQQKPGQAPRLLIYDASNRATGIPARFSGSGSGTDFTLTISSLEPEDFAVYYCQQYFHWPPTFGGGTKVEIK

SEQ ID NO: 616 - protein, 5 aa, synthetic construct
  REGION 1..5: note = Synthetic Construct
SYRMH

SEQ ID NO: 617 - protein, 17 aa, synthetic construct
  REGION 1..17: note = Synthetic Construct
FIVPSGGSTGYAQKFQG

SEQ ID NO: 618 - protein, 11 aa, synthetic construct
  REGION 1..11: note = Synthetic Construct
VSRYAPEPMDV

SEQ ID NO: 619 - protein, 11 aa, synthetic construct
  REGION 1..11: note = Synthetic Construct
RASQGISRWLA

SEQ ID NO: 620 - protein, 7 aa, synthetic construct
  REGION 1..7: note = Synthetic Construct
AASSLQS

SEQ ID NO: 621 - protein, 9 aa, synthetic construct
  REGION 1..9: note = Synthetic Construct
QQAFSHPWT

SEQ ID NO: 622 - protein, 9 aa, synthetic construct
  REGION 1..9: note = Synthetic Construct
GGSISSSSY

SEQ ID NO: 623 - protein, 5 aa, synthetic construct
  REGION 1..5: note = Synthetic Construct
YYSGS

SEQ ID NO: 624 - protein, 20 aa, synthetic construct
  REGION 1..20: note = Synthetic Construct
DSGIGYSYASSHGYYYYMDV

SEQ ID NO: 625 - protein, 11 aa, synthetic construct
  REGION 1..11: note = Synthetic Construct
RASQSVGSNLA

SEQ ID NO: 626 - protein, 7 aa, synthetic construct
  REGION 1..7: note = Synthetic Construct
GASTRAT

SEQ ID NO: 627 - protein, 9 aa, synthetic construct
  REGION 1..9: note = Synthetic Construct
QQYFHFPLT

SEQ ID NO: 628 - protein, 4 aa, synthetic construct
  REGION 1..4: note = Synthetic Construct
YGSG

SEQ ID NO: 629 - protein, 9 aa, synthetic construct
  REGION 1..9: note = Synthetic Construct
GGSIGSGGY

SEQ ID NO: 630 - protein, 7 aa, synthetic construct
  REGION 1..7: note = Synthetic Construct
GFTFSSY

SEQ ID NO: 631 - protein, 6 aa, synthetic construct
  REGION 1..6: note = Synthetic Construct
SGSGGS

SEQ ID NO: 632 - protein, 12 aa, synthetic construct
  REGION 1..12: note = Synthetic Construct
DPLSLLLGYFDY

SEQ ID NO: 633 - protein, 11 aa, synthetic construct
  REGION 1..11: note = Synthetic Construct
RASQSISSWLA

SEQ ID NO: 634 - protein, 7 aa, synthetic construct
  REGION 1..7: note = Synthetic Construct
DASSLES

SEQ ID NO: 635 - protein, 9 aa, synthetic construct
  REGION 1..9: note = Synthetic Construct
QQYNRHSPT

SEQ ID NO: 636 - protein, 12 aa, synthetic construct
  REGION 1..12: note = Synthetic Construct
DPLSLLLGYFDY

SEQ ID NO: 637 - protein, 6 aa, synthetic construct
  REGION 1..6: note = Synthetic Construct
SGSGGA

SEQ ID NO: 638 - protein, 7 aa, synthetic construct
  REGION 1..7: note = Synthetic Construct
GFTFSRY

SEQ ID NO: 639 - protein, 11 aa, synthetic construct
  REGION 1..11: note = Synthetic Construct
AGKYRWHGMDV

SEQ ID NO: 640 - protein, 11 aa, synthetic construct
  REGION 1..11: note = Synthetic Construct
RASQGISSWLA

SEQ ID NO: 641 - protein, 7 aa, synthetic construct
  REGION 1..7: note = Synthetic Construct
AASNLQS

SEQ ID NO: 642 - protein, 9 aa, synthetic construct
  REGION 1..9: note = Synthetic Construct
QQAPDLPIT

SEQ ID NO: 643 - protein, 5 aa, synthetic construct
  REGION 1..5: note = Synthetic Construct
VHSGH

SEQ ID NO: 644 - protein, 8 aa, synthetic construct
  REGION 1..8: note = Synthetic Construct
GYSISSGV

SEQ ID NO: 645 - protein, 5 aa, synthetic construct
  REGION 1..5: note = Synthetic Construct
SYGMT

SEQ ID NO: 646 - protein, 17 aa, synthetic construct
  REGION 1..17: note = Synthetic Construct
VISGSGGGTYYADSVKG

SEQ ID NO: 647 - protein, 9 aa, synthetic construct
  REGION 1..9: note = Synthetic Construct
GPRIVGMDV

SEQ ID NO: 648 - protein, 11 aa, synthetic construct
  REGION 1..11: note = Synthetic Construct
RASQGISSWLA

SEQ ID NO: 649 - protein, 7 aa, synthetic construct
  REGION 1..7: note = Synthetic Construct
AASSLQS

SEQ ID NO: 650 - protein, 9 aa, synthetic construct
  REGION 1..9: note = Synthetic Construct
QQVFSYPLT

SEQ ID NO: 651 - protein, 7 aa, synthetic construct
  REGION 1..7: note = Synthetic Construct
GYTFTSY

SEQ ID NO: 652 - protein, 6 aa, synthetic construct
  REGION 1..6: note = Synthetic Construct
SPYNGN

SEQ ID NO: 653 - protein, 11 aa, synthetic construct
  REGION 1..11: note = Synthetic Construct
VSMYAPEPMDV

SEQ ID NO: 654 - protein, 11 aa, synthetic construct
  REGION 1..11: note = Synthetic Construct
RASQGISRWLA

SEQ ID NO: 655 - protein, 6 aa, synthetic construct
  REGION 1..6: note = Synthetic Construct
ASSLQS

SEQ ID NO: 656 - protein, 9 aa, synthetic construct
  REGION 1..9: note = Synthetic Construct
QQAFSHPWT

SEQ ID NO: 657 - protein, 11 aa, synthetic construct
  REGION 1..11: note = Synthetic Construct
VSMYAPEPMDV

SEQ ID NO: 658 - protein, 6 aa, synthetic construct
  REGION 1..6: note = Synthetic Construct
NPASGW

SEQ ID NO: 659 - protein, 10 aa, synthetic construct
  REGION 1..10: note = Synthetic Construct
GYTFTGHKMH

SEQ ID NO: 660 - protein, 9 aa, synthetic construct
  REGION 1..9: note = Synthetic Construct
GGSISSGSY

SEQ ID NO: 661 - protein, 5 aa, synthetic construct
  REGION 1..5: note = Synthetic Construct
YYSGS

SEQ ID NO: 662 - protein, 12 aa, synthetic construct
  REGION 1..12: note = Synthetic Construct
GLGMYYHVPFDI

SEQ ID NO: 663 - protein, 11 aa, synthetic construct
  REGION 1..11: note = Synthetic Construct
RASQSVSSYLA

SEQ ID NO: 664 - protein, 7 aa, synthetic construct
  REGION 1..7: note = Synthetic Construct
DASNRAT

SEQ ID NO: 665 - protein, 9 aa, synthetic construct
  REGION 1..9: note = Synthetic Construct
QQYFHWPPT

SEQ ID NO: 666 - protein, 13 aa, synthetic construct
  REGION 1..13: note = Synthetic Construct
WIGGIYPSGSTYY

SEQ ID NO: 667 - protein, 5 aa, synthetic construct
  REGION 1..5: note = Synthetic Construct
YYSGK

SEQ ID NO: 668 - protein, 6 aa, synthetic construct
  REGION 1..6: note = Synthetic Construct
YYSGRT

SEQ ID NO: 669 - protein, 9 aa, synthetic construct
  REGION 1..9: note = Synthetic Construct
GGSIKSGGY

SEQ ID NO: 670 - protein, 9 aa, synthetic construct
  REGION 1..9: note = Synthetic Construct
GGSILSGGY

SEQ ID NO: 671 - protein, 9 aa, synthetic construct
  REGION 1..9: note = Synthetic Construct
GGSISSGGY

SEQ ID NO: 672 - protein, 9 aa, synthetic construct
  REGION 1..9: note = Synthetic Construct
GGSIESGGY

SEQ ID NO: 673 - protein, 11 aa, synthetic construct
  REGION 1..11: note = Synthetic Construct
VSMYAPEPMDV

SEQ ID NO: 674 - protein, 11 aa, synthetic construct
  REGION 1..11: note = Synthetic Construct
VSMYAPEPMDV

SEQ ID NO: 675 - protein, 11 aa, synthetic construct
  REGION 1..11: note = Synthetic Construct
VSRYAPEPMDV

SEQ ID NO: 676 - protein, 6 aa, synthetic construct
  REGION 1..6: note = Synthetic Construct
RPSVGS

SEQ ID NO: 677 - protein, 6 aa, synthetic construct
  REGION 1..6: note = Synthetic Construct
VPSGGS

SEQ ID NO: 678 - protein, 6 aa, synthetic construct
  REGION 1..6: note = Synthetic Construct
VPSGGS

SEQ ID NO: 679 - protein, 7 aa, synthetic construct
  REGION 1..7: note = Synthetic Construct
GYTFTSY

SEQ ID NO: 680 - protein, 7 aa, synthetic construct
  REGION 1..7: note = Synthetic Construct
GYTFTSY

SEQ ID NO: 681 - protein, 5 aa, synthetic construct
  REGION 1..5: note = Synthetic Construct
NYWMG

SEQ ID NO: 682 - protein, 17 aa, synthetic construct
  REGION 1..17: note = Synthetic Construct
YIRSSGGLTHYADSVKG

SEQ ID NO: 683 - protein, 7 aa, synthetic construct
  REGION 1..7: note = Synthetic Construct
GLYSFDY

SEQ ID NO: 684 - protein, 11 aa, synthetic construct
  REGION 1..11: note = Synthetic Construct
RASQGVNNFLA

SEQ ID NO: 685 - protein, 7 aa, synthetic construct
  REGION 1..7: note = Synthetic Construct
AASTLQS

SEQ ID NO: 686 - protein, 9 aa, synthetic construct
  REGION 1..9: note = Synthetic Construct
QQSYSTPRG

SEQ ID NO: 687 - protein, 115 aa, synthetic construct [sequence omitted]

SEQ ID NO: 688 - protein, 111 aa, synthetic construct [sequence omitted]

SEQ ID NO: 689 - protein, 119 aa, synthetic construct
  REGION 1..119: note = Synthetic Construct
QLQLQESGPGLVKPSETLSLTCTVSGGSISSSSYYWGWIRQPPGKGLEWIGSISYSGSTYYNPSLKSRVTISVDTSKNQFSLKLSSVTAADTAVYYCARDSLRYGMDVWGQGTTVTVSS

SEQ ID NO: 690 - protein, 114 aa, synthetic construct
  REGION 1..114: note = Synthetic Construct
DIVMTQSPDSLAVSLGERATINCKSSQSVLYSSNNKNYLAWYQQKPGQPPKLLIYWASTRESGVPDRFSGSGSGTDFTLTISSLQAEDVAVYYCQQYALAPPRTFGGGTKVEIK

SEQ ID NO: 691 - protein, 107 aa, synthetic construct
  REGION 1..107: note = Synthetic Construct
EIVMTQSPATLSVSPGERATLSCRASQSVSSNLAWYQQKPGQAPRLLIYGASTRATGIPARFSGSGSGTEFTLTISSLQSEDFAVYYCQVYNVWPRTFGGGTKVEIK

SEQ ID NO: 692 - protein, 118 aa, synthetic construct
  REGION 1..118: note = Synthetic Construct
EVQLLESGGGLVQPGGSLRLSCAASGFTFGSYGMTWVRQAPGKGLEWVSVISGSGGGTYYADSVKGRFTISRDNSKNTLYLQMNSLRAEDTAVYYCAKGPRVVGMDVWGQGTTVTVSS

SEQ ID NO: 693 - protein, 107 aa, synthetic construct
  REGION 1..107: note = Synthetic Construct
DIQMTQSPSSVSASVGDRVTITCRASQGISSWLAWYQQKPGKAPKLLIYAASSLQSGVPSRFSGSGSGTDYTLTISSLQPEDFATYYCQQVFSYPLTFGGGTKVEIK

SEQ ID NO: 694 - protein, 108 aa, synthetic construct
  REGION 1..108: note = Synthetic Construct
DIQMTQSPSTLSASVGDRVTITCRASQSIGSWLAWYQQKPGKAPKLLIYKASSLESGVPSRFSGSGSGTEFTLTISSLQPDDFATYYCQVYGSYSPRTFGGGTKVEIK

SEQ ID NO: 695 - protein, 119 aa, synthetic construct
  REGION 1..119: note = Synthetic Construct
EVQLLESGGGLVQPGGSLRLSCAASGFTFSSYAMSWVRQAPGKGLEWVSAISGSGGSTSYADSVKGRFTISRDNSKNTLYLQMNSLRAEDTAVYYCARLPQYSRPFDYWGQGTLVTVSS

SEQ ID NO: 696 - protein, 107 aa, synthetic construct
  REGION 1..107: note = Synthetic Construct
DIQMTQSPSSVSASVGDRVTITCRASQGISSWLAWYQQKPGKAPKLLIYAASSLQSGVPSRFSGSGSGTDFTLTISSLQPEDFATYYCQQGHSFPLTFGGGTKVEIK

SEQ ID NO: 697 - protein, 120 aa, synthetic construct
  REGION 1..120: note = Synthetic Construct
QVQLVQSGAEVKKPGASVKVSCKASGYTFTSYRMHWVRQAPGQGLEWMGFIVPSGGSTSYAQKFQGRVTMTRDTSTSTVYMELSSLRSEDTAVYYCARVSRYAPEPMDVWGQGTTVTVSS

SEQ ID NO: 698 - protein, 107 aa, synthetic construct; identical to SEQ ID NO: 592
  REGION 1..107: note = Synthetic Construct

SEQ ID NO: 699 - protein, 11 aa, synthetic construct
  REGION 1..11: note = Synthetic Construct
GSISSSSYYWG

SEQ ID NO: 700 - protein, 16 aa, synthetic construct
  REGION 1..16: note = Synthetic Construct
SISYSGSTYYNPSLKS

SEQ ID NO: 701 - protein, 11 aa, synthetic construct
  REGION 1..11: note = Synthetic Construct
ARDSLRYGMDV

SEQ ID NO: 702 - protein, 17 aa, synthetic construct
  REGION 1..17: note = Synthetic Construct
KSSQSVLYSSNNKNYLA

SEQ ID NO: 703 - protein, 7 aa, synthetic construct
  REGION 1..7: note = Synthetic Construct
WASTRES

SEQ ID NO: 704 - protein, 10 aa, synthetic construct
  REGION 1..10: note = Synthetic Construct
QQYALAPPRT

SEQ ID NO: 705 - protein, 11 aa, synthetic construct
  REGION 1..11: note = Synthetic Construct
RASQSVSSNLA

SEQ ID NO: 706 - protein, 7 aa, synthetic construct
  REGION 1..7: note = Synthetic Construct
GASTRAT

SEQ ID NO: 707 - protein, 9 aa, synthetic construct
  REGION 1..9: note = Synthetic Construct
QVYNVWPRT

SEQ ID NO: 708 - protein, 5 aa, synthetic construct
  REGION 1..5: note = Synthetic Construct
SYGMT

SEQ ID NO: 709 - protein, 17 aa, synthetic construct
  REGION 1..17: note = Synthetic Construct
VISGSGGGTYYADSVKG

SEQ ID NO: 710 - protein, 11 aa, synthetic construct
  REGION 1..11: note = Synthetic Construct
AKGPRVVGMDV

SEQ ID NO: 711 - protein, 11 aa, synthetic construct
  REGION 1..11: note = Synthetic Construct
RASQGISSWLA

SEQ ID NO: 712 - protein, 7 aa, synthetic construct
  REGION 1..7: note = Synthetic Construct
AASSLQS

SEQ ID NO: 713 - protein, 9 aa, synthetic construct
  REGION 1..9: note = Synthetic Construct
QQVFSYPLT

SEQ ID NO: 714 - protein, 11 aa, synthetic construct
  REGION 1..11: note = Synthetic Construct
RASQSIGSWLA

SEQ ID NO: 715 - protein, 7 aa, synthetic construct
  REGION 1..7: note = Synthetic Construct
KASSLES

SEQ ID NO: 716 - protein, 10 aa, synthetic construct
  REGION 1..10: note = Synthetic Construct
QVYGSYSPRT

SEQ ID NO: 717 - protein, 7 aa, synthetic construct
  REGION 1..7: note = Synthetic Construct
GFTFSSY

SEQ ID NO: 718 - protein, 17 aa, synthetic construct
  REGION 1..17: note = Synthetic Construct
AISGSGGSTSYADSVKG

SEQ ID NO: 719 - protein, 12 aa, synthetic construct
  REGION 1..12: note = Synthetic Construct
ARLPQYSRPFDY

SEQ ID NO: 720 - protein, 11 aa, synthetic construct
RASQGISSWLA

SEQ ID NO: 721 - protein, 7 aa, synthetic construct
  REGION 1..7: note = Synthetic Construct
AASSLQS

SEQ ID NO: 722 - protein, 9 aa, synthetic construct
  REGION 1..9: note = Synthetic Construct
QQGHSFPLT

SEQ ID NO: 723 - protein, 5 aa, synthetic construct
  REGION 1..5: note = Synthetic Construct
SYRMH

SEQ ID NO: 724 - protein, 17 aa, synthetic construct
  REGION 1..17: note = Synthetic Construct
FIVPSGGSTSYAQKFQG

SEQ ID NO: 725 - protein, 11 aa, synthetic construct
  REGION 1..11: note = Synthetic Construct
VSRYAPEPMDV

SEQ ID NO: 726 - protein, 11 aa, synthetic construct
  REGION 1..11: note = Synthetic Construct
RASQGISRWLA

SEQ ID NO: 727 - protein, 7 aa, synthetic construct
  REGION 1..7: note = Synthetic Construct
AASSLQS

SEQ ID NO: 728 - protein, 9 aa, synthetic construct
  REGION 1..9: note = Synthetic Construct
QQAFSHPWT

SEQ ID NO: 729 - protein, 115 aa, Homo sapiens
ILGRSETQECLFFNANWEKDRTNQTGVEPCYGDKDKRRHCFATWKNISGSIEIVKQGCWLDDINCYDRTDCVEKKDSPEVYFCCCEGNMCNEKFSYFPEMEVTQPTSNPVTPKPP

SEQ ID NO: 730 - protein, 115 aa, synthetic construct
  REGION 1..115: note = Synthetic Construct
  VARIANT 11: note = Xaa is IIe or Leu
  VARIANT 12: note = Xaa is Phe or Tyr
  VARIANT 19: note = Xaa is Leu or Lys
  VARIANT 20: note = Xaa is Asp or Glu
  VARIANT 25: note = Xaa is Thr or Ser
  VARIANT 27: note = Xaa is Leu or Val
  VARIANT 29: note = Xaa is Pro or Arg
  VARIANT 31: note = Xaa is Tyr or Glu
  VARIANT 33: note = Xaa is Asp or Glu
  VARIANT 34: note = Xaa is Lys or Gln
  VARIANT 38: note = Xaa is Arg or Leu
  VARIANT 41: note = Xaa is Tyr or Phe
  VARIANT 45: note = Xaa is Arg or Lys
  VARIANT 47: note = Xaa is Ser or Ile
  VARIANT 48: note = Xaa is Ser or Thr
  VARIANT 50: note = Xaa is Ser or Thr
  VARIANT 51: note = Xaa is Ile or Leu
  VARIANT 53: note = Xaa is Ile or Leu
  VARIANT 56: note = Xaa is Lys or Gln
  VARIANT 63: note = Xaa is Phe or Ile
  VARIANT 69: note = Xaa is Gln, Thr, or Asp
  VARIANT 74: note = Xaa is Lys or Thr
  VARIANT 75: note = Xaa is Lys or Glu
  VARIANT 76: note = Xaa is Asp or Glu
  VARIANT 77: note = Xaa is Ser or Asn
  VARIANT 79: note = Xaa is Glu or Gln
  VARIANT 89: note = Xaa is Phe or Met
  VARIANT 70: note = Xaa is Glu, Asp, or Thr
GRGEAETRECXXYNANWEXXRTNQXGXEXCXGXXDKRXHCXASWXNXXGXXEXVKXGCWLDDXNCYDRXXCVAXXXXPXVYFCCCEGNXCNERFTHLPEAGGPEVTYEPPPTAPT

SEQ ID NO: 731 - protein, 115 aa, synthetic construct
  REGION 1..115: note = Synthetic Construct
GRGEAETRECIFYNANWEKDRTNQSGLEPCYGDQDKRRHCFASWKNSSGTIELVKQGCWLDDINCYDRQECVAKKDSPEVYFCCCEGNFCNERFTHLPEAGGPEVTYEPPPTAPT

SEQ ID NO: 732 - protein, 115 aa, synthetic construct
  REGION 1..115: note = Synthetic Construct
GRGEAETRECIYYNANWELDRTNQSGLERCEGEQDKRLHCYASWRNSSGTIELVKKGCWLDDINCYDRQECVATKENPQVYFCCCEGNFCNERFTHLPEAGGPEVTYEPPPTAPT

SEQ ID NO: 733 - protein, 115 aa, synthetic construct
  REGION 1..115: note = Synthetic Construct
GRGEAETRECIYYNANWELERTNQSGLERCEGEQDKRLHCYASWRNSSGTIELVKKGCWLDDFNCYDRQECVAKKDSPEVYFCCCEGNFCNERFTHLPEAGGPEVTYEPPPTAPT

SEQ ID NO: 734 - protein, 115 aa, synthetic construct
  REGION 1..115: note = Synthetic Construct
GRGEAETRECIYYNANWELERTNQTGLERCEGEQDKRLHCYASWRNISGTIELVKKGCWLDDFNCYDRQECVAKKDSPEVYFCCCEGNFCNERFTHLPEAGGPEVTYEPPPTAPT

SEQ ID NO: 735 - protein, 115 aa, synthetic construct
  REGION 1..115: note = Synthetic Construct
GRGEAETRECIYYNANWELERTNQTGLERCEGEQDKRLHCYASWRNITGTIELVKKGCWLDDFNCYDRQECVAKKDSPEVYFCCCEGNFCNERFTHLPEAGGPEVTYEPPPTAPT

SEQ ID NO: 736 - protein, 115 aa, synthetic construct
  REGION 1..115: note = Synthetic Construct
GRGEAETRECIYYNANWELERTNQSGLEPCEGEQDKRLHCYASWRNSSGTIELVKKGCWLDDFNCYDRQECVAKKDSPEVYFCCCEGNFCNERFTHLPEAGGPEVTYEPPPTAPT

SEQ ID NO: 737 - protein, 115 aa, synthetic construct
  REGION 1..115: note = Synthetic Construct
GRGEAETRECIYYNANWELERTNQSGLERCYGDKDKRLHCYASWRNSSGTIELVKKGCWLDDFNCYDRQECVAKKDSPEVYFCCCEGNFCNERFTHLPEAGGPEVTYEPPPTAPT

SEQ ID NO: 738 - protein, 115 aa, synthetic construct
  REGION 1..115: note = Synthetic Construct
GRGEAETRECIYYNANWELERTNQSGLERCEGEQDKRLHCFASWKNSSGTIELVKQGCWLDDFNCYDRQECVAKKDSPEVYFCCCEGNFCNERFTHLPEAGGPEVTYEPPPTAPT

SEQ ID NO: 739 - protein, 115 aa, synthetic construct
  REGION 1..115: note = Synthetic Construct
GRGEAETRECIFYNANWEKDRTNQSGLERCEGEQDKRLHCYASWRNSSGTIELVKKGCWLDDFNCYDRQECVAKKDSPEVYFCCCEGNFCNERFTHLPEAGGPEVTYEPPPTAPT

SEQ ID NO: 740 - protein, 115 aa, synthetic construct
  REGION 1..115: note = Synthetic Construct
GRGEAETRECIYYNANWELERTNQSGLERCYGDQDKRRHCYASWRNSSGTIELVKKGCWLDDFNCYDRQECVAKKDSPEVYFCCCEGNFCNERFTHLPEAGGPEVTYEPPPTAPT

SEQ ID NO: 741 - protein, 115 aa, synthetic construct
  REGION 1..115: note = Synthetic Construct
GRGEAETRECLYYNANWELERTNQSGVERCEGEKDKRLHCYASWRNSSGSLEIVKKGCWLDDFNCYDRTDCVATEENPQVYFCCCEGNMCNERFTHLPEAGGPEVTYEPPPTAPT

SEQ ID NO: 742 - protein, 115 aa, synthetic construct
  REGION 1..115: note = Synthetic Construct
GRGEAETRECLYYNANWELERTNQSGVERCEGEKDKRLHCYASWRNSSGSLEIVKKGCWLDDFNCYDRDTCVATEENPQVYFCCCEGNMCNERFTHLPEAGGPEVTYEPPPTAPT

SEQ ID NO: 743 - protein, 115 aa, synthetic construct
  REGION 1..115: note = Synthetic Construct
GRGEAETRECLYYNANWELERTNQSGVERCEGEKDKRLHCYASWRNSSGSLEIVKKGCWLDDFNCYDRTDCVATKENPQVYFCCCEGNMCNERFTHLPEAGGPEVTYEPPPTAPT

SEQ ID NO: 744 - protein, 115 aa, synthetic construct
  REGION 1..115: note = Synthetic Construct
GRGEAETRECLYYNANWELERTNQSGVERCEGEKDKRLHCYASWRNSSGSLEIVKKGCWLDDFNCYDRDTCVATKENPQVYFCCCEGNMCNERFTHLPEAGGPEVTYEPPPTAPT

SEQ ID NO: 745 - protein, 110 aa, synthetic construct
  REGION 1..110: note = Synthetic Construct
ETRECIYYNANWELERTNQSGLERCYGDKDKRRHCYASWRNSSGTIELVKKGCWLDDFNCYDRQECVATEENPQVYFCCCEGNFCNERFTHLPEAGGPEVTYEPPPTAPT

SEQ ID NO: 746 - protein, 110 aa, synthetic construct
  REGION 1..110: note = Synthetic Construct
ETRECIYYNANWELERTNQSGLERCEGDQDKRLHCYASWRNSSGTIELVKKGCWLDDINCYDRQECVATKENPQVYFCCCEGNFCNERFTHLPEAGGPEVTYEPPPTAPT

SEQ ID NO: 747 - protein, 107 aa, synthetic construct
  REGION 1..107: note = Synthetic Construct
ETRECIYYNANWELERTNQSGLERCEGEQDKRLHCYASWRNSSGTIELVKKGCWDDDFNCYDRQECVATEENPQVYFCCCEGNFCNERFTHLPEAGGPEVTYEPPPT

SEQ ID NO: 748 - protein, 110 aa, synthetic construct
  REGION 1..110: note = Synthetic Construct
ETRWCIYYNANWELERTNQSGLERCEGEQDKRLHCYASWRNSSGTIELVKKGCWLDDFNCYDRQECVATEENPQVYFCCCEGNFCNERFTHLPEAGGPEVTYEPPPTAPT

SEQ ID NO: 749 - protein, 110 aa, synthetic construct
  REGION 1..110: note = Synthetic Construct
ETRWCIYYNANWELERTNQTGLERCEGEQDKRLHCYASWRNSSGTIELVKKGCWLDDFNCYDRQECVATEENPQVYFCCCEGNFCNERFTHLPEAGGPEVTYEPPPTAPT

SEQ ID NO: 750 - protein, 110 aa, synthetic construct
  REGION 1..110: note = Synthetic Construct
ETRYCIYYNANWELERTNQTGLERCEGEQDKRLHCYASWRNSSGTIELVKKGCWLDDFNCYDRQECVATEENPQVYFCCCEGNFCNERFTHLPEAGGPEVTYEPPPTAPT

SEQ ID NO: 751 - protein, 109 aa, synthetic construct
  REGION 1..109: note = Synthetic Construct
  VARIANT 37: note = Xaa is Asp or Arg
  VARIANT 65: note = Xaa is Ile, Phe, Glu, Asp, Tyr, Ser, Asn, Gln, or Thr
  VARIANT 66: note = Xaa is Asn or Thr
  VARIANT 75: note = Xaa is Ala or Glu
  VARIANT 76: note = Xaa is Thr or Lys
  VARIANT 77: note = Xaa is Glu or Lys
  VARIANT 78: note = Xaa is Glu or Asp
  VARIANT 79: note = Xaa is Asn or Ser
  VARIANT 81: note = Xaa is Gln, Glu, Lys, Arg, Asp, or Asn
GAILGRAETRECIYYNANWELERTNQSGLERCEGEQXKRRHCFATWKNISGSIEIVKQGCWLDDXXCYDRTDCVXXXXXPXVYFCCCEGNMCNEKFSYFPEMEVTQPTS

SEQ ID NO: 752 - protein, 109 aa, synthetic construct
  REGION 1..109: note = Synthetic Construct
  VARIANT 37: note = Xaa is Asp or Arg
  VARIANT 65: note = Xaa is Ile, Phe, Glu, Asp, Tyr, Ser, Asn, Gln, or Thr
  VARIANT 66: note = Xaa is Asn or Thr
  VARIANT 75: note = Xaa is Ala or Glu
  VARIANT 76: note = Xaa is Thr or Lys
  VARIANT 77: note = Xaa is Glu or Lys
  VARIANT 78: note = Xaa is Glu or Asp
  VARIANT 79: note = Xaa is Asn or Ser
  VARIANT 81: note = Xaa is Gln, Glu, Lys, Arg, Asp, or Asn
GAILGRAETRECIYYNANWELERTNQSGLERCEGEQXKRLHCFATWKNISGSIEIVKQGCWLDDXXCYDRTDCVXXXXXPXVYFCCCEGNMCNEKFSYFPEMEVTQPTS

SEQ ID NO: 753 - protein, 109 aa, synthetic construct
  REGION 1..109: note = Synthetic Construct
  VARIANT 37: note = Xaa is Asp or Arg
  VARIANT 65: note = Xaa is Ile, Phe, Glu, Asp, Tyr, Ser, Asn, Gln, or Thr
  VARIANT 66: note = Xaa is Asn or Thr
  VARIANT 75: note = Xaa is Ala or Glu
  VARIANT 76: note = Xaa is Thr or Lys
  VARIANT 77: note = Xaa is Glu or Lys
  VARIANT 78: note = Xaa is Glu or Asp
  VARIANT 79: note = Xaa is Asn or Ser
  VARIANT 81: note = Xaa is Gln, Glu, Lys, Arg, Asp, or Asn
GAILGRAETRECIYYNANWELERTNQSGLERCEGEQXKRLHCYASWKNISGSIEIVKQGCWLDDXXCYDRTDCVXXXXXPXVYFCCCEGNMCNEKFSYFPEMEVTQPTS

SEQ ID NO: 754 - protein, 109 aa, synthetic construct
  REGION 1..109: note = Synthetic Construct
  VARIANT 37: note = Xaa is Asp or Arg
  VARIANT 65: note = Xaa is Ile, Phe, Glu, Asp, Tyr, Ser, Asn, Gln, or Thr
  VARIANT 66: note = Xaa is Asn or Thr
  VARIANT 75: note = Xaa is Ala or Glu
  VARIANT 76: note = Xaa is Thr or Lys
  VARIANT 77: note = Xaa is Glu or Lys
  VARIANT 78: note = Xaa is Glu or Asp
  VARIANT 79: note = Xaa is Asn or Ser
  VARIANT 81: note = Xaa is Gln, Glu, Lys, Arg, Asp, or Asn
GAILGRAETRECIYYNANWELERTNQSGLERCEGEQXKRLHCYASWRNSSGSIEIVKQGCWLDDXXCYDRTDCVXXXXXPXVYFCCCEGNMCNEKFSYFPEMEVTQPTS

SEQ ID NO: 755 - protein, 109 aa, synthetic construct
  REGION 1..109: note = Synthetic Construct
  VARIANT 37: note = Xaa is Asp or Arg
  VARIANT 65: note = Xaa is Ile, Phe, Glu, Asp, Tyr, Ser, Asn, Gln, or Thr
  VARIANT 66: note = Xaa is Asn or Thr
  VARIANT 75: note = Xaa is Ala or Glu
  VARIANT 76: note = Xaa is Thr or Lys
  VARIANT 78: note = Xaa is Glu or Asp
  VARIANT 79: note = Xaa is Asn or Ser
  VARIANT 81: note = Xaa is Gln, Glu, Lys, Arg, Asp, or Asn
  VARIANT 77: note = Xaa is Glu or Lys
GAILGRAETRECIYYNANWELERTNQSGLERCEGEQXKRLHCYASWRNSSGTIEIVKQGCWLDDXXCYDRTDCVXXXXXPXVYFCCCEGNMCNEKFSYFPEMEVTQPTS

SEQ ID NO: 756 - protein, 109 aa, synthetic construct
  REGION 1..109: note = Synthetic Construct
  VARIANT 37: note = Xaa is Asp or Arg
  VARIANT 65: note = Xaa is Ile, Phe, Glu, Asp, Tyr, Ser, Asn, Gln, or Thr
  VARIANT 66: note = Xaa is Asn or Thr
  VARIANT 75: note = Xaa is Ala or Glu
  VARIANT 76: note = Xaa is Thr or Lys
  VARIANT 77: note = Xaa is Glu or Lys
  VARIANT 78: note = Xaa is Glu or Asp
  VARIANT 79: note = Xaa is Asn or Ser
  VARIANT 81: note = Xaa is Gln, Glu, Lys, Arg, Asp, or Asn
GAILGRAETRECIYYNANWELERTNQSGLERCEGEQXKRLHCYASWRNSSGTIELVKKGCWLDDXXCYDRTDCVXXXXXPXVYFCCCEGNMCNEKFSYFPEMEVTQPTS

SEQ ID NO: 757 - protein, 109 aa, synthetic construct
  REGION 1..109: note = Synthetic Construct
  VARIANT 37: note = Xaa is Asp or Arg
  VARIANT 65: note = Xaa is Ile, Phe, Glu, Asp, Tyr, Ser, Asn, Gln, or Thr
  VARIANT 66: note = Xaa is Asn or Thr
  VARIANT 75: note = Xaa is Ala or Glu
  VARIANT 76: note = Xaa is Thr or Lys
  VARIANT 77: note = Xaa is Glu or Lys
  VARIANT 78: note = Xaa is Glu or Asp
  VARIANT 79: note = Xaa is Asn or Ser
  VARIANT 81: note = Xaa is Gln, Glu, Lys, Arg, Asp, or Asn
GAILGRAETRECIYYNANWELERTNQSGLERCEGEQXKRLHCYASWRNSSGTIELVKKGCWLDDXXCYDRQECVXXXXXPXVYFCCCEGNMCNEKFSYFPEMEVTQPTS

SEQ ID NO: 758 - protein, 107 aa, synthetic construct
  REGION 1..107: note = Synthetic Construct
  VARIANT 63: note = Xaa is Ile, Phe, Glu, Asp, Tyr, Ser, Asn, Gln, or Thr
  VARIANT 64: note = Xaa is Asn or Thr
  VARIANT 73: note = Xaa is Ala or Glu
  VARIANT 74: note = Xaa is Thr or Lys
  VARIANT 75: note = Xaa is Glu or Lys
  VARIANT 76: note = Xaa is Glu or Asp
  VARIANT 77: note = Xaa is Asn or Ser
  VARIANT 79: note = Xaa is Gln, Glu, Lys, Arg, Asp, or Asn
  VARIANT 35: note = Xaa is Asp or Arg
GRGEAETRECIYYNANWELERTNQSGLERCEGEQXKRRHCFATWKNISGSIEIVKQGCWLDDXXCYDRTDCVXXXXXPXVYFCCCEGNMCNEKFSYFPEMEVTQPTS

SEQ ID NO: 759 - protein, 107 aa, synthetic construct
  REGION 1..107: note = Synthetic Construct
  VARIANT 35: note = Xaa is Asp or Arg
  VARIANT 63: note = Xaa is Ile, Phe, Glu, Asp, Tyr, Ser, Asn, Gln, or Thr
  VARIANT 64: note = Xaa is Asn or Thr
  VARIANT 73: note = Xaa is Ala or Glu
  VARIANT 74: note = Xaa is Thr or Lys
  VARIANT 75: note = Xaa is Glu or Lys
  VARIANT 76: note = Xaa is Glu or Asp
  VARIANT 79: note = Xaa is Gln, Glu, Lys, Arg, Asp, or Asn
  VARIANT 77: note = Xaa is Asn or Ser
GRGEAETRECIYYNANWELERTNQSGLERCEGEQXKRLHCFATWKNISGSIEIVKQGCWLDDXXCYDRTDCVXXXXXPXVYFCCCEGNMCNEKFSYFPEMEVTQPTS

SEQ ID NO: 760 - protein, 107 aa, synthetic construct
  REGION 1..107: note = Synthetic Construct
  VARIANT 35: note = Xaa is Asp or Arg
  VARIANT 63: note = Xaa is Ile, Phe, Glu, Asp, Tyr, Ser, Asn, Gln, or Thr
  VARIANT 64: note = Xaa is Asn or Thr
  VARIANT 73: note = Xaa is Ala or Glu
  VARIANT 74: note = Xaa is Thr or Lys
  VARIANT 75: note = Xaa is Glu or Lys
  VARIANT 76: note = Xaa is Glu or Asp
  VARIANT 77: note = Xaa is Asn or Ser
  VARIANT 79: note = Xaa is Gln, Glu, Lys, Arg, Asp, or Asn
GRGEAETRECIYYNANWELERTNQSGLERCEGEQXKRLHCYASWKNISGSIEIVKQGCWLDDXXCYDRTDCVXXXXXPXVYFCCCEGNMCNEKFSYFPEMEVTQPTS

SEQ ID NO: 761 - protein, 107 aa, synthetic construct
  REGION 1..107: note = Synthetic Construct
  VARIANT 35: note = Xaa is Asp or Arg
  VARIANT 64: note = Xaa is Asn or Thr
  VARIANT 73: note = Xaa is Ala or Glu
  VARIANT 74: note = Xaa is Thr or Lys
  VARIANT 75: note = Xaa is Glu or Lys
  VARIANT 76: note = Xaa is Glu or Asp
  VARIANT 77: note = Xaa is Asn or Ser
  VARIANT 79: note = Xaa is Gln, Glu, Lys, Arg, Asp, or Asn
  VARIANT 63: note = Xaa is Ile, Phe, Glu, Asp, Tyr, Ser, Asn, Gln, or Thr
GRGEAETRECIYYNANWELERTNQSGLERCEGEQXKRLHCYASWRNSSGSIEIVKQGCWLDDXXCYDRTDCVXXXXXPXVYFCCCEGNMCNEKFSYFPEMEVTQPTS

SEQ ID NO: 762 - protein, 107 aa, synthetic construct
  REGION 1..107: note = Synthetic Construct
  VARIANT 35: note = Xaa is Asp or Arg
  VARIANT 63: note = Xaa is Ile, Phe, Glu, Asp, Tyr, Ser, Asn, Gln, or Thr
  VARIANT 64: note = Xaa is Asn or Thr
  VARIANT 73: note = Xaa is Ala or Glu
  VARIANT 74: note = Xaa is Thr or Lys
  VARIANT 76: note = Xaa is Glu or Asp
  VARIANT 77: note = Xaa is Asn or Ser
  VARIANT 79: note = Xaa is Gln, Glu, Lys, Arg, Asp, or Asn
  VARIANT 75: note = Xaa is Glu or Lys
GRGEAETRECIYYNANWELERTNQSGLERCEGEQXKRLHCYASWRNSSGTIEIVKQGCWLDDXXCYDRTDCVXXXXXPXVYFCCCEGNMCNEKFSYFPEMEVTQPTS

SEQ ID NO: 763 - protein, 107 aa, synthetic construct
  REGION 1..107: note = Synthetic Construct
  VARIANT 35: note = Xaa is Asp or Arg
  VARIANT 63: note = Xaa is Ile, Phe, Glu, Asp, Tyr, Ser, Asn, Gln, or Thr
  VARIANT 64: note = Xaa is Asn or Thr
  VARIANT 73: note = Xaa is Ala or Glu
  VARIANT 74: note = Xaa is Thr or Lys
  VARIANT 75: note = Xaa is Glu or Lys
  VARIANT 76: note = Xaa is Glu or Asp
  VARIANT 77: note = Xaa is Asn or Ser
  VARIANT 79: note = Xaa is Gln, Glu, Lys, Arg, Asp, or Asn
GRGEAETRECIYYNANWELERTNQSGLERCEGEQXKRLHCYASWRNSSGTIELVKKGCWLDDXXCYDRTDCVXXXXXPXVYFCCCEGNMCNEKFSYFPEMEVTQPTS

SEQ ID NO: 764 - protein, 107 aa, synthetic construct
  REGION 1..107: note = Synthetic Construct
  VARIANT 35: note = Xaa is Asp or Arg
  VARIANT 63: note = Xaa is Ile, Phe, Glu, Asp, Tyr, Ser, Asn, Gln, or Thr
  VARIANT 64: note = Xaa is Asn or Thr
  VARIANT 73: note = Xaa is Ala or Glu
  VARIANT 74: note = Xaa is Thr or Lys
  VARIANT 75: note = Xaa is Glu or Lys
  VARIANT 76: note = Xaa is Glu or Asp
  VARIANT 77: note = Xaa is Asn or Ser
  VARIANT 79: note = Xaa is Gln, Glu, Lys, Arg, Asp, or Asn
GRGEAETRECIYYNANWELERTNQSGLERCEGEQXKRLHCYASWRNSSGTIELVKKGCWLDDXXCYDRQECVXXXXXPXVYFCCCEGNMCNEKFSYFPEMEVTQPTS

SEQ ID NO: 765 - protein, 109 aa, synthetic construct
  REGION 1..109: note = Synthetic Construct
  VARIANT 37: note = Xaa is Asp or Arg
  VARIANT 65: note = Xaa is Ile, Phe, Glu, Asp, Tyr, Ser, Asn, Gln, or Thr
  VARIANT 66: note = Xaa is Asn or Thr
  VARIANT 76: note = Xaa is Thr or Lys
  VARIANT 77: note = Xaa is Glu or Lys
  VARIANT 78: note = Xaa is Glu or Asp
  VARIANT 79: note = Xaa is Asn or Ser
  VARIANT 81: note = Xaa is Gln, Glu, Lys, Arg, Asp, or Asn
  VARIANT 75: note = Xaa is Ala or Glu
GAILGRSETQECIYYNANWELERTNQSGLERCEGEQXKRRHCFATWKNISGSIEIVKQGCWLDDXXCYDRTDCVXXXXXPXVYFCCCEGNMCNEKFSYFPEMEVTQPTS

SEQ ID NO: 766 - protein, 109 aa, synthetic construct
  REGION 1..109: note = Synthetic Construct
  VARIANT 37: note = Xaa is Asp or Arg
  VARIANT 65: note = Xaa is Ile, Phe, Glu, Asp, Tyr, Ser, Asn, Gln, or Thr
  VARIANT 66: note = Xaa is Asn or Thr
  VARIANT 75: note = Xaa is Ala or Glu
  VARIANT 76: note = Xaa is Thr or Lys
  VARIANT 77: note = Xaa is Glu or Lys
  VARIANT 78: note = Xaa is Glu or Asp
  VARIANT 79: note = Xaa is Asn or Ser
  VARIANT 81: note = Xaa is Gln, Glu, Lys, Arg, Asp, or Asn
GAILGRSETQECIYYNANWELERTNQSGLERCEGEQXKRLHCFATWKNISGSIEIVKQGCWLDDXXCYDRTDCVXXXXXPXVYFCCCEGNMCNEKFSYFPEMEVTQPTS

SEQ ID NO: 767 - protein, 109 aa, synthetic construct
  REGION 1..109: note = Synthetic Construct
  VARIANT 37: note = Xaa is Asp or Arg
  VARIANT 65: note = Xaa is Ile, Phe, Glu, Asp, Tyr, Ser, Asn, Gln, or Thr
  VARIANT 66: note = Xaa is Asn or Thr
  VARIANT 75: note = Xaa is Ala or Glu
  VARIANT 76: note = Xaa is Thr or Lys
  VARIANT 77: note = Xaa is Glu or Lys
  VARIANT 78: note = Xaa is Glu or Asp
  VARIANT 79: note = Xaa is Asn or Ser
  VARIANT 81: note = Xaa is Gln, Glu, Lys, Arg, Asp, or Asn
GAILGRSETQECIYYNANWELERTNQSGLERCEGEQXKRLHCYASWKNISGSIEIVKQGCWLDDXXCYDRTDCVXXXXXPXVYFCCCEGNMCNEKFSYFPEMEVTQPTS

SEQ ID NO: 768 - protein, 109 aa, synthetic construct
  REGION 1..109: note = Synthetic Construct
  VARIANT 37: note = Xaa is Asp or Arg
  VARIANT 65: note = Xaa is Ile, Phe, Glu, Asp, Tyr, Ser, Asn, Gln, or Thr
  VARIANT 66: note = Xaa is Asn or Thr
  VARIANT 75: note = Xaa is Ala or Glu
  VARIANT 76: note = Xaa is Thr or Lys
  VARIANT 77: note = Xaa is Glu or Lys
  VARIANT 78: note = Xaa is Glu or Asp
  VARIANT 79: note = Xaa is Asn or Ser
  VARIANT 81: note = Xaa is Gln, Glu, Lys, Arg, Asp, or Asn
GAILGRSETQECIYYNANWELERTNQSGLERCEGEQXKRLHCYASWRNSSGSIEIVKQGCWLDDXXCYDRTDCVXXXXXPXVYFCCCEGNMCNEKFSYFPEMEVTQPTS

SEQ ID NO: 769 - protein, 109 aa, synthetic construct
  REGION 1..109: note = Synthetic Construct
  VARIANT 37: note = Xaa is Asp or Arg
  VARIANT 65: note = Xaa is Ile, Phe, Glu, Asp, Tyr, Ser, Asn, Gln, or Thr
  VARIANT 66: note = Xaa is Asn or Thr
  VARIANT 75: note = Xaa is Ala or Glu
  VARIANT 76: note = Xaa is Thr or Lys
  VARIANT 77: note = Xaa is Glu or Lys
  VARIANT 78: note = Xaa is Glu or Asp
  VARIANT 79: note = Xaa is Asn or Ser
  VARIANT 81: note = Xaa is Gln, Glu, Lys, Arg, Asp, or Asn
GAILGRSETQECIYYNANWELERTNQSGLERCEGEQXKRLHCYASWRNSSGTIEIVKQGCWLDDXXCYDRTDCVXXXXXPXVYFCCCEGNMCNEKFSYFPEMEVTQPTS

SEQ ID NO: 770 - protein, 109 aa, synthetic construct
  REGION 1..109: note = Synthetic Construct
  VARIANT 37: note = Xaa is Asp or Arg
  VARIANT 65: note = Xaa is Ile, Phe, Glu, Asp, Tyr, Ser, Asn, Gln, or Thr
  VARIANT 66: note = Xaa is Asn or Thr
  VARIANT 76: note = Xaa is Thr or Lys
  VARIANT 77: note = Xaa is Glu or Lys
  VARIANT 78: note = Xaa is Glu or Asp
  VARIANT 81: note = Xaa is Gln, Glu, Lys, Arg, Asp, or Asn
  VARIANT 79: note = Xaa is Asn or Ser
  VARIANT 75: note = Xaa is Ala or Glu
GAILGRSETQECIYYNANWELERTNQSGLERCEGEQXKRLHCYASWRNSSGTIELVKKGCWLDDXXCYDRTDCVXXXXXPXVYFCCCEGNMCNEKFSYFPEMEVTQPTS

SEQ ID NO: 771 - protein, 109 aa, synthetic construct
  REGION 1..109: note = Synthetic Construct
  VARIANT 37: note = Xaa is Asp or Arg
  VARIANT 65: note = Xaa is Ile, Phe, Glu, Asp, Tyr, Ser, Asn, Gln, or Thr
  VARIANT 66: note = Xaa is Asn or Thr
  VARIANT 75: note = Xaa is Ala or Glu
  VARIANT 76: note = Xaa is Thr or Lys
  VARIANT 77: note = Xaa is Glu or Lys
  VARIANT 78: note = Xaa is Glu or Asp
  VARIANT 79: note = Xaa is Asn or Ser
  VARIANT 81: note = Xaa is Gln, Glu, Lys, Arg, Asp, or Asn
GAILGRSETQECIYYNANWELERTNQSGLERCEGEQXKRLHCYASWRNSSGTIELVKKGCWLDDXXCYDRQECVXXXXXPXVYFCCCEGNMCNEKFSYFPEMEVTQPTS

SEQ ID NO: 772 - protein, 109 aa, synthetic construct
  REGION 1..109: note = Synthetic Construct
GAILGRAETRECIYYNANWELERTNQSGLERCEGEQDKRLHCYASWRNSSGTIEIVKQGCWLDDINCYDRTDCVATEENPQVYFCCCEGNMCNEKFSYFPEMEVTQPTS

SEQ ID NO: 773 - protein, 107 aa, synthetic construct
  REGION 1..107: note = Synthetic Construct
GRGEAETRECIYYNANWELERTNQSGLERCEGEQDKRLHCYASWRNSSGTIEIVKQGCWLDDINCYDRTDCVEKKDSPEVYFCCCEGNMCNEKFSYFPEMEVTQPTS

SEQ ID NO: 774 - protein, 107 aa, synthetic construct
  REGION 1..107: note = Synthetic Construct
GRGEAETRECIYYNANWELERTNQSGLERCEGEQDKRLHCYASWRNSSGTIEIVKQGCWLDDINCYDRTDCVEKKDSPQVYFCCCEGNMCNEKFSYFPEMEVTQPTS

SEQ ID NO: 775 - protein, 107 aa, synthetic construct
  REGION 1..107: note = Synthetic Construct
GRGEAETRECIYYNANWELERTNQSGLERCEGEQDKRLHCYASWRNSSGTIEIVKQGCWLDDFNCYDRTDCVEKKDSPQVYFCCCEGNMCNEKFSYFPEMEVTQPTS

SEQ ID NO: 776 - protein, 109 aa, synthetic construct
  REGION 1..109: note = Synthetic Construct
GAILGRAETRECIYYNANWELERTNQSGLERCEGEQDKRLHCYASWRNSSGTIEIVKQGCWLDDFNCYDRTDCVATEENPQVYFCCCEGNMCNEKFSYFPEMEVTQPTS

SEQ ID NO: 777 - protein, 107 aa, synthetic construct
  REGION 1..107: note = Synthetic Construct
GRGEAETRECIYYNANWELERTNQSGLERCEGEQDKRLHCYASWRNSSGTIEIVKQGCWLDDFNCYDRTDCVEKKDSPEVYFCCCEGNMCNEKFSYFPEMEVTQPTS

SEQ ID NO: 778 - protein, 107 aa, synthetic construct
  REGION 1..107: note = Synthetic Construct
GRGEAETRECIYYNANWELERTNQSGLERCEGEQDKRRHCFATWKNISGSIEIVKQGCWLDDFNCYDRTDCVEKKDSPQVYFCCCEGNMCNEKFSYFPEMEVTQPTS

SEQ ID NO: 779 - protein, 107 aa, synthetic construct
  REGION 1..107: note = Synthetic Construct
GRGEAETRECIYYNANWELERTNQSGLERCEGEQDKRLHCFATWKNISGSIEIVKQGCWLDDFNCYDRTDCVEKKDSPQVYFCCCEGNMCNEKFSYFPEMEVTQPTS

SEQ ID NO: 780 - protein, 107 aa, synthetic construct
  REGION 1..107: note = Synthetic Construct
GRGEAETRECIYYNANWELERTNQSGLERCEGEQDKRLHCYASWKNISGSIEIVKQGCWLDDFNCYDRTDCVEKKDSPQVYFCCCEGNMCNEKFSYFPEMEVTQPTS

SEQ ID NO: 781 - protein, 107 aa, synthetic construct
  REGION 1..107: note = Synthetic Construct
GRGEAETRECIYYNANWELERTNQSGLERCEGEQDKRLHCYASWRNSSGSIEIVKQGCWLDDFNCYDRTDCVEKKDSPQVYFCCCEGNMCNEKFSYFPEMEVTQPTS

SEQ ID NO: 782 - protein, 107 aa, synthetic construct
  REGION 1..107: note = Synthetic Construct
GRGEAETRECIYYNANWELERTNQSGLERCEGEQDKRLHCYASWRNSSGTIELVKKGCWLDDFNCYDRTDCVEKKDSPQVYFCCCEGNMCNEKFSYFPEMEVTQPTS

SEQ ID NO: 783 - protein, 107 aa, synthetic construct
  REGION 1..107: note = Synthetic Construct
GRGEAETRECIYYNANWELERTNQSGLERCEGEQDKRLHCYASWRNSSGTIELVKKGCWLDDFNCYDRQECVATKDSPQVYFCCCEGNMCNEKFSYFPEMEVTQPTS

SEQ ID NO: 784 - protein, 107 aa, synthetic construct
  REGION 1..107: note = Synthetic Construct
GRGEAETRECIYYNANWELERTNQSGLERCEGEQDKRLHCYASWRNSSGTIELVKKGCWLDDFNCYDRQECVATEENPQVYFCCCEGNMCNEKFSYFPEMEVTQPTS

SEQ ID NO: 785 - protein, 107 aa, synthetic construct
  REGION 1..107: note = Synthetic Construct
GRGEAETRECIYYNANWELERTNQSGLERCEGEQDKRLHCYASWRNSSGTIEIVKQGCWLDDNNCYDRTDCVEKKDSPQVYFCCCEGNMCNEKFSYFPEMEVTQPTS

SEQ ID NO: 786 - protein, 107 aa, synthetic construct
  REGION 1..107: note = Synthetic Construct
GRGEAETRECIYYNANWELERTNQSGLERCEGEQDKRLHCYASWRNSSGTIEIVKQGCWLDDTNCYDRTDCVEKKDSPQVYFCCCEGNMCNEKFSYFPEMEVTQPTS

SEQ ID NO: 787 - protein, 107 aa, synthetic construct
  REGION 1..107: note = Synthetic Construct
GRGEAETRECIYYNANWELERTNQSGLERCEGEQDKRLHCYASWRNSSGTIEIVKQGCWLDDETCYDRTDCVEKKDSPQVYFCCCEGNMCNEKFSYFPEMEVTQPTS

SEQ ID NO: 788 - protein, 107 aa, synthetic construct
  REGION 1..107: note = Synthetic Construct
GRGEAETRECIYYNANWELERTNQSGLERCEGEQRKRLHCYASWRNSSGTIEIVKQGCWLDDFNCYDRTDCVEKKDSPQVYFCCCEGNMCNEKFSYFPEMEVTQPTS

SEQ ID NO: 789 - protein, 107 aa, synthetic construct
  REGION 1..107: note = Synthetic Construct
GRGEAETRECIYYNANWELERTNQSGLERCEGEQDKRLHCYASWRNSSGTIEIVKQGCWLDDFNCYDRTDCVETKDSPQVYFCCCEGNMCNEKFSYFPEMEVTQPTS

SEQ ID NO: 790 - protein, 109 aa, synthetic construct
  REGION 1..109: note = Synthetic Construct
GAILGRAETRECIYYNANWELERTNQSGLERCEGEQDKRLHCYASWRNSSGTIEIVKQGCWLDDFNCYDRTDCVEKKDSPQVYFCCCEGNMCNEKFSYFPEMEVTQPTS

SEQ ID NO: 791 - protein, 109 aa, synthetic construct
  REGION 1..109: note = Synthetic Construct
GAILGRSETQECIYYNANWELERTNQSGLERCEGEQDKRLHCYASWRNSSGTIEIVKQGCWLDDFNCYDRTDCVEKKDSPQVYFCCCEGNMCNEKFSYFPEMEVTQPTS

SEQ ID NO: 792 - protein, 107 aa, synthetic construct
  REGION 1..107: note = Synthetic Construct
GRGEAETRECIYYNANWELERTNQSGLERCEGEQDKRLHCYASWRNSSGTIELVKKGCWLDDFNCYDRQECVATKDSPEVYFCCCEGNMCNEKFSYFPEMEVTQPTS

SEQ ID NO: 793 - protein, 107 aa, synthetic construct
  REGION 1..107: note = Synthetic Construct
GRGEAETRECIYYNANWELERTNQSGLERCEGEQDKRLHCYASWRNSSGTIELVKKGCWLDDFNCYDRQECVATKENPQVYFCCCEGNMCNEKFSYFPEMEVTQPTS

SEQ ID NO: 794 - protein, 10 aa, synthetic construct
  REGION 1..10: note = Synthetic Construct
GAILGRSETQ

SEQ ID NO: 795 - protein, 8 aa, synthetic construct
  REGION 1..8: note = Synthetic Construct
GRGEAETR

SEQ ID NO: 796 - protein, 6 aa, synthetic construct
  REGION 1..6: note = Synthetic Construct
ECLFFN

SEQ ID NO: 797 - protein, 6 aa, synthetic construct
  REGION 1..6: note = Synthetic Construct
ECIYYN

SEQ ID NO: 798 - protein, 9 aa, synthetic construct
  REGION 1..9: note = Synthetic Construct
ANWEKDRTN

SEQ ID NO: 799 - protein, 9 aa, synthetic construct
  REGION 1..9: note = Synthetic Construct
ANWELERTN

SEQ ID NO: 800 - protein, 7 aa, synthetic construct
  REGION 1..7: note = Synthetic Construct
QTGVEPC

SEQ ID NO: 801 - protein, 7 aa, synthetic construct
  REGION 1..7: note = Synthetic Construct
QSGLERC

SEQ ID NO: 802 - protein, 7 aa, synthetic construct
  REGION 1..7: note = Synthetic Construct
YGDKDKR

SEQ ID NO: 803 - protein, 7 aa, synthetic construct
  REGION 1..7: note = Synthetic Construct
EGEQDKR

SEQ ID NO: 804 - protein, 10 aa, synthetic construct
  REGION 1..10: note = Synthetic Construct
RHCFATWKNI

SEQ ID NO: 805 - protein, 7 aa, synthetic construct
  REGION 1..7: note = Synthetic Construct
HCFATWK

SEQ ID NO: 806 - protein, 10 aa, synthetic construct
  REGION 1..10: note = Synthetic Construct
LHCYASWRNS

SEQ ID NO: 807 - protein, 7 aa, synthetic construct
  REGION 1..7: note = Synthetic Construct
HCYASWR

SEQ ID NO: 808 - protein, 10 aa, synthetic construct
  REGION 1..10: note = Synthetic Construct
SIEIVKQGCW

SEQ ID NO: 809 - protein, 8 aa, synthetic construct
  REGION 1..8: note = Synthetic Construct
EIVKQGCW

SEQ ID NO: 810 - protein, 10 aa, synthetic construct
  REGION 1..10: note = Synthetic Construct
TIELVKKGCW

SEQ ID NO: 811 - protein, 8 aa, synthetic construct
  REGION 1..8: note = Synthetic Construct
ELVKKGCW

SEQ ID NO: 812 - protein, 12 aa, synthetic construct
  REGION 1..12: note = Synthetic Construct
LDDINCYDRTDC

SEQ ID NO: 813 - protein, 12 aa, synthetic construct
  REGION 1..12: note = Synthetic Construct
LDDFNCYDRQEC

SEQ ID NO: 814 - protein, 6 aa, synthetic construct
  REGION 1..6: note = Synthetic Construct
KDSPEV

SEQ ID NO: 815 - protein, 6 aa, synthetic construct
  REGION 1..6: note = Synthetic Construct
EENPQV

SEQ ID NO: 816 - protein, 6 aa, synthetic construct
  REGION 1..6: note = Synthetic Construct
YFCCCE

SEQ ID NO: 817 - protein, 6 aa, synthetic construct
  REGION 1..6: note = Synthetic Construct
GNMCNE

SEQ ID NO: 818 - protein, 6 aa, synthetic construct
  REGION 1..6: note = Synthetic Construct
GNFCNE

SEQ ID NO: 819 - protein, 5 aa, synthetic construct
  REGION 1..5: note = Synthetic Construct
KFSYF

SEQ ID NO: 820 - protein, 5 aa, synthetic construct
  REGION 1..5: note = Synthetic Construct
RFTHL

SEQ ID NO: 821 - protein, 10 aa, synthetic construct
  REGION 1..10: note = Synthetic Construct
PEMEVTQPTS

SEQ ID NO: 822 - protein, 18 aa, synthetic construct
  REGION 1..18: note = Synthetic Construct
PEAGGPEVTYEPPPTAPT

SEQ ID NO: 823 - skipped

SEQ ID NO: 824 - protein, 16 aa, synthetic construct
  REGION 1..16: note = Synthetic Construct
  VARIANT 4: note = Xaa is Tyr or His
  VARIANT 7: note = Xaa is Ser or Lys
  VARIANT 8: note = Xaa is Thr, Met or Lys
  VARIANT 10: note = Xaa is Tyr or Lys
  VARIANT 12: note = Xaa is Ser or Glu
  VARIANT 16: note = Xaa is Gly or Lys
  VARIANT 11: note = Xaa is Ala, Met or Glu
IISXAGXXYXXXWAKX

SEQ ID NO: 825 - protein, 11 aa, synthetic construct
  REGION 1..11: note = Synthetic Construct
  VARIANT 5: note = Xaa is Tyr or His
  VARIANT 7: note = Xaa is Thr or His
  VARIANT 11: note = Xaa is Leu or Lys
GVPAXSXGGDX

SEQ ID NO: 826 - protein, 13 aa, synthetic construct
  REGION 1..13: note = Synthetic Construct
  VARIANT 1: note = Xaa is Gln or Thr
  VARIANT 2: note = Xaa is Ser or Thr
  VARIANT 5: note = Xaa is Ser or Glu
  VARIANT 7: note = Xaa is Tyr or Phe
  VARIANT 8: note = Xaa is Asp or His
  VARIANT 9: note = Xaa is Asn, Asp, Ala or Glu
XXSQXVXXXNWLS

SEQ ID NO: 827 - protein, 7 aa, synthetic construct
  REGION 1..7: note = Synthetic Construct
  VARIANT 3: note = Xaa is Ser or Glu
  VARIANT 7: note = Xaa is Ser, Tyr, Phe or Trp
WAXTLAX

SEQ ID NO: 828 - protein, 10 aa, synthetic construct
  REGION 1..10: note = Synthetic Construct
  VARIANT 8: note = Xaa is Leu or Arg
AGGYGGGXYA

(19) United States
(12) Patent Application Publication
(10) Pub. No.: US 2024/0218061 A1
(43) Pub. Date: Jul. 4, 2024

(54) METHODS OF USING ACTIVIN RECEPTOR TYPE II SIGNALING INHIBITORS

(71) Applicant: Keros Therapeutics, Inc., Lexington, MA (US)

(72) Inventors: Jasbir S. SEEHRA, Lexington, MA (US); Jennifer LACHEY, Lincoln, MA (US); Christopher R. ROVALDI, Swampscott, MA (US); Elissa FURUTANI, Belmont, MA (US)

(21) Appl. No.: 18/390,085

(22) Filed: Dec. 20, 2023

Related U.S. Application Data
(63) Continuation of application No. PCT/US22/34366, filed on Jun. 21, 2022.
(60) Provisional application No. 63/350,745, filed on Jun. 9, 2022.
(60) Provisional application No. 63/341,843, filed on May 13, 2022.
(60) Provisional application No. 63/274,993, filed on Nov. 3, 2021.
(60) Provisional application No. 63/287,810, filed on Dec. 9, 2021.
(60) Provisional application No. 63/213,123, filed on Jun. 21, 2021.
(60) Provisional application No. 63/235,091, filed on Aug. 19, 2021.

(51) Int. Cl. C07K 16/22; A61P 7/06; A61P 35/00; C07K 14/71; C07K 16/28

(52) U.S. Cl. CPC C07K 16/22; A61P 7/06; A61P 35/00; C07K 14/71; C07K 16/2863; C07K 2317/76

(57) ABSTRACT

The invention features methods of treating a subject having a cytopenia associated with a myelodysplastic syndrome, a cytopenia associated with chronic myelomonocytic leukemia, or a cytopenia associated with myelofibrosis, such as anemia, thrombocytopenia, or neutropenia, by administering to the subject an ActRII signaling inhibitor, such as an ActRIIA ligand trap including an extracellular ActRIIA variant. The extracellular ActRIIA variant may be fused to an Fc domain or moiety.

### SEQUENCE LISTING

The instant application contains a Sequence Listing which has been submitted electronically in XML file format and is hereby incorporated by reference in its entirety. Said XML copy, created on Mar. 21, 2024, is named 51184-040008_Sequence_Listing_3_21_24.xml and is 1,204,607 bytes in size.

### BACKGROUND OF THE INVENTION

Myelodysplastic syndromes, or MDS, is a collection of bone marrow disorders characterized by ineffective hematopoiesis, often with a dramatic expansion of progenitor cells that are unable to mature into functioning blood cells. In the United States, there are 60,000 to 170,000 patients with MDS and 15,000 to 20,000 new cases of MDS reported each year. MDS predominantly affects older adults, with approximately 75% of patients aged 60 years or older at diagnosis. Median survival ranges from approximately nine years for very low-risk patients to less than a year for high-risk patients. Anemia is the most frequent consequence of ineffective hematopoiesis in patients with MDS due to low red blood cell production, impacting 90% of MDS patients. Another consequence is thrombocytopenia, which affects 40-65% of patients. Patients with MDS-associated anemia are generally treated with red blood cell transfusions and erythropoiesis stimulating agents (ESAs), which are not approved for such treatment. MDS-associated thrombocytopenia is typically treated with platelet transfusions and platelet stimulating agents.

Myelofibrosis is a chronic myeloproliferative malignancy characterized by clonal proliferation of myeloid cells and megakaryocytic hyperplasia/dysplasia resulting in bone marrow fibrosis and osteosclerosis. It can present as a de novo disorder (primary myelofibrosis, PMF) or evolve from polycythemia vera (post-PV MF), essential thrombocythemia (post-ET MF), myelodysplastic syndrome (MDS), lupus, or other hematologic and solid tumors. Myeloproliferative neoplasms arise from a single somatically mutated hematopoietic stem cell progenitor that clonally expands and gives rise to virtually all myeloid cells and B and natural killer cells. It is characterized by bone marrow fibrosis, ineffective hematopoiesis, splenomegaly, extramedullary hematopoiesis, constitutional symptoms, and shortened survival. Extensive scarring in the bone marrow in subjects with myelofibrosis can lead to severe anemia and a low number of platelets. Symptoms of myelofibrosis include fatigue, bone pain, easy bruising, easy bleeding, and fever. There are no curative medical therapies for patients with myelofibrosis, but JAK inhibitors, such as ruxolitinib (JAKAFI®/JAKAVI®), fedratinib (INREBIC®), and pacritinib (VONJO™) have been shown to reduce spleen volume and improve symptoms associated with myelofibrosis. However, JAK inhibitors interfere with normal hematopoiesis and treatment with ruxolitinib and fedratinib is complicated by the development of anemia and thrombocytopenia, which can lead to dose reductions and reduced adherence, thereby limiting the number of patients able to remain on JAK inhibitors. Patients with aggressive or high-risk myelofibrosis may require a blood transfusion or bone marrow transplant. Other treatment options include therapies that have known risks, such as androgen therapy and treatment with thalidomide or related medications. For patients with intermediate-risk myelofibrosis, treatment is typically directed at symptom management.

Chronic myelomonocytic leukemia (CMML) is an uncommon blood cancer that generally affects older adults. It features an accumulation of immature monocytes, which can disrupt the production of red blood cells and platelets and lead to the development of anemia and thrombocytopenia. Subjects with CMML may also have a lack of neutrophils and an enlarged spleen. About 15% to 30% of people with CMML develop acute myeloid leukemia (AML). Currently, there are limited treatment options for CMML-associated cytopenias, in particular in subjects who are ring sideroblast positive with a high transfusion burden.

Accordingly, there exists a need for novel and effective treatments for MDS-associated, CMML-associated, and myelofibrosis-associated cytopenias.

### SUMMARY OF THE INVENTION

The present invention features ActRII signaling inhibitors, including activin A antibodies, activin B antibodies, myostatin antibodies, GDF-11 antibodies, ActRII antibodies, and ActRII ligand traps, including ActRIIA ligand traps that include an extracellular activin receptor type IIA (ActRIIA) variant. In some embodiments, an ActRIIA ligand trap includes an extracellular ActRIIA variant fused to the N- or C-terminus of an Fc domain, Fc domain monomer, or other moiety. Such moieties may be attached by amino acid or other covalent bonds and may increase stability of the polypeptide. An ActRIIA ligand trap including an extracellular ActRIIA variant fused to an Fc domain monomer may also form a dimer (e.g., a homodimer or heterodimer) through the interaction between two Fc domain monomers. The ActRII signaling inhibitors of the invention may be used to treat a subject having or at risk of developing a cytopenia (e.g., anemia, thrombocytopenia, or neutropenia) associated with a myelodysplastic syndrome (an MDS-associated cytopenia), chronic myelomonocytic leukemia, or myelofibrosis, for example, by increasing hemoglobin levels, increasing hematocrit, increasing red blood cell count, promoting or increasing the maturation and/or differentiation of erythroid progenitors, increasing late-stage erythroid precursor maturation, recruiting early-stage progenitors into the erythroid lineage, increasing proerythroblast numbers, increasing reticulocytes, increasing early-stage erythroid precursor and/or progenitor numbers, promoting the progression of erythroid precursors and/or progenitors through erythropoiesis, increasing platelet levels (e.g., increasing platelet count), increasing neutrophil levels (e.g., increasing neutrophil count), reducing transfusion burden, and/or promoting transfusion independence in a subject having a myelodysplastic syndrome, chronic myelomonocytic leukemia, or myelofibrosis.

Exemplary embodiments of the invention are described in the enumerated paragraphs below.
E1. A method of treating a subject having or at risk of developing a cytopenia associated with a myelodysplastic syndrome who has not received prior treatment with azacitidine, decitabine, lenalidomide, luspatercept, or sotatercept by administering to the subject a therapeutically effective amount of an ActRII signaling inhibitor.
E2. A method of treating a cytopenia in a subject having a myelodysplastic syndrome who has not received prior treatment with azacitidine, decitabine, lenalidomide, luspatercept, or sotatercept by administering to the subject a therapeutically effective amount of an ActRII signaling inhibitor.
E3. A method of treating a subject having or at risk of developing a cytopenia associated with a myelodysplastic syndrome who has or who is identified as having an erythropoietin level greater than 100 mlU/mL by administering to the subject a therapeutically effective amount of an ActRII signaling inhibitor.
E4 A method of treating a cytopenia in a subject having a myelodysplastic syndrome who has or who is identified as having an erythropoietin level greater than 100 mlU/mL by administering to the subject a therapeutically effective amount of an ActRII signaling inhibitor.
E5. A method of promoting transfusion independence in a subject in need thereof by administering to the subject a therapeutically effective amount of an ActRII signaling inhibitor.
E6. The method of E5, wherein the subject has or is at risk of developing a cytopenia associated with a myelodysplastic syndrome.
E7. The method of E5, wherein the subject has or is at risk of developing a cytopenia associated with chronic myelomonocytic leukemia (CMML).
E8. The method of E5, wherein the subject has or is at risk of developing a cytopenia associated with myelofibrosis.
E9. The method of any one of E1-E4 and E6, wherein the myelodysplastic syndrome is myelodysplastic syndrome with unilineage dysplasia (MDS-SLD), myelodysplastic syndrome with multilineage dysplasia (MDS-MLD), myelodysplastic syndrome with ring sideroblasts (MDS-RS, which includes single lineage dysplasia (MDS-RS-SLD) and multilineage dysplasia (MDS-RS-MLD)), myelodysplastic syndrome associated with isolated del chromosome abnormality (myelodysplastic syndrome with isolated del(5q)), myelodysplastic syndrome with excess blasts (e.g., myelodysplastic syndrome with excess blasts—type 1 (MDS-EB-1) or myelodysplastic syndrome with excess blasts—type 2 (MDS-EB-2)), myelodysplastic syndrome, unclassifiable (MDS-U), or myelodysplastic syndrome/myeloproliferative neoplasm with ring sideroblasts and thrombocytosis (MDS/MPN-RS-T).
E10. The method of any one of E1-E4, E6, and E9, wherein the myelodysplastic syndrome is MDS-SLD.
E11. The method of any one of E1-E4, E6, and E9, wherein the myelodysplastic syndrome is MDS-MLD.
E12. The method of any one of E1-E4, E6, and E9, wherein the myelodysplastic syndrome is MDS-RS-SLD.
E13. The method of any one of E1-E4, E6, and E9, wherein the myelodysplastic syndrome is MDS-RS-MLD.
E14. The method of any one of E1-E4, E6, and E9, wherein the myelodysplastic syndrome is myelodysplastic syndrome with isolated del(5q).
E15. The method of any one of E1-E4, E6, and E9, wherein the myelodysplastic syndrome is MDS-EB-1.
E16. The method of any one of E1-E4, E6, and E9, wherein the myelodysplastic syndrome is MDS-EB-2.
E17. The method of any one of E1-E4, E6, and E9, wherein the myelodysplastic syndrome is MDS-U.
E18. The method of any one of E1-E4, E6, and E9, wherein the myelodysplastic syndrome is MDS/MPN-RS-T.
E19. The method of any one of E1-E4, E6 and E9-E18, wherein the myelodysplastic syndrome is a ring sideroblast positive myelodysplastic syndrome (RS positive MDS, e.g., the subject has ring sideroblasts).
E20. The method of E19, wherein the RS-positive myelodysplastic syndrome is associated with a splicing factor mutation.
E21. The method of E20, wherein the splicing factor mutation is a mutation in Splicing Factor 3b Subunit 1 (SF3B1).
E22. The method of any one of E1-E4, E6, E9-E11, and E14-E17, wherein the myelodysplastic syndrome is a non-ring sideroblast myelodysplastic syndrome (non-RS, e.g., the subject lacks ring sideroblasts).
E23. The method of any one of E1-E4, E6 and E9-E22, wherein the myelodysplastic syndrome is a very low, low, or intermediate risk myelodysplastic syndrome (e.g., as determined by the Revised International Prognostic Scoring System).
E24. The method of E23, wherein the myelodysplastic syndrome is a very low risk myelodysplastic syndrome (e.g., as determined by the Revised International Prognostic Scoring System).
E25. The method of E23, wherein the myelodysplastic syndrome is a low-risk myelodysplastic syndrome (e.g., as determined by the Revised International Prognostic Scoring System).
E26. The method of E23, wherein the myelodysplastic syndrome is an intermediate risk myelodysplastic syndrome (e.g., as determined by the Revised International Prognostic Scoring System).
E27. The method of any one of E1-E4, E6 and E9-E26, wherein the myelodysplastic syndrome is associated with a defect in terminal maturation.
E28. The method of any one of E1-E4, E6 and E9-E26, wherein the myelodysplastic syndrome is associated with a defect in early-stage hematopoiesis (e.g., commitment or differentiation of progenitor cells).
E29. The method of any one of E1-E4, E6 and E9-E28, wherein the myelodysplastic syndrome is associated with elevated endogenous erythropoietin levels.
E30. The method of any one of E1-E4, E6 and E9-E29, wherein the myelodysplastic syndrome is associated with hypocellular bone marrow (e.g., the subject has hypocellular bone marrow).
E31. A method of treating a subject having a cytopenia associated with CMML, comprising administering to the subject a therapeutically effective amount of an ActRII signaling inhibitor.
E32. A method of treating a cytopenia in a subject having CMML, comprising administering to the subject a therapeutically effective amount of an ActRII signaling inhibitor.
E33. The method of E31 or E32, wherein the subject has not received prior treatment with azacitidine, decitabine, lenalidomide, luspatercept, or sotatercept.
E34. The method of any one of E31-E33, wherein the subject has ineffective hematopoiesis.
E35. The method of any one of E7 and E31-E34, wherein the CMML is CMML-0
E36. A method of treating a subject having a cytopenia associated with primary myelofibrosis (PMF), post-essential thrombocythemia myelofibrosis (post-ET MF), or post-polycythemia vera myelofibrosis (post-PV MF), comprising administering to the subject an effective amount of an ActRII signaling inhibitor.
E37. A method of treating a cytopenia in a subject having PMF, post-ET MF, or post-PV MF, comprising administering to the subject an effective amount of an ActRII signaling inhibitor.
E38. A method of treating a subject having PMF, post-ET MF, or post-PV MF, comprising administering to the subject an effective amount of an ActRII signaling inhibitor.
E39. A method of reducing osteosclerosis in a subject having myelofibrosis, comprising administering to the subject an effective amount of an ActRII signaling inhibitor.
E40. The method of E39, wherein the method further comprises evaluating osteosclerosis after administration of the ActRII signaling inhibitor.
E41. The method of E39, wherein the subject is identified as having osteosclerosis prior to administration of the ActRII signaling inhibitor.
E42. The method of E39, wherein the method further comprises identifying the subject as having osteosclerosis prior to administration of the ActRII signaling inhibitor.
E43. A method of reducing splenomegaly in a subject having myelofibrosis, comprising administering to the subject an effective amount of an ActRII signaling inhibitor.
E44. A method of reducing splenomegaly associated with extramedullary hematopoiesis in a subject in need thereof, comprising administering to the subject an effective amount of an ActRII signaling inhibitor.
E45. The method of E43 or E44, wherein the method further comprises evaluating spleen volume after administration of the ActRII signaling inhibitor.
E46. The method of E43 or E44, wherein the subject is identified as having splenomegaly prior to administration of the ActRII signaling inhibitor.
E47. The method of E43 or E44, wherein the method further comprises identifying the subject as having splenomegaly prior to administration of the ActRII signaling inhibitor.
E48. A method of reducing bone marrow fibrosis in a subject having myelofibrosis, comprising administering to the subject an effective amount of an ActRII signaling inhibitor.
E49. The method of E48, wherein the method further comprises evaluating bone marrow fibrosis after administration of the ActRII signaling inhibitor.
E50. The method of E48, wherein the subject is identified as having bone marrow fibrosis prior to administration of the ActRII signaling inhibitor.
E51. The method of E48, wherein the method further comprises identifying the subject as having bone marrow fibrosis prior to administration of the ActRII signaling inhibitor.
E52. A method of reducing platelet number or platelet volume in a subject having myelofibrosis, thrombocythemia, or polycythemia vera, or in a subject that requires phlebotomy due to excess red blood cells, comprising administering to the subject an effective amount of an ActRII signaling inhibitor.
E53. The method of E52, wherein the method further comprises evaluating platelet number or platelet volume after administration of the ActRII signaling inhibitor.
E54. The method of E52, wherein the subject is identified as having high platelet levels prior to administration of the ActRII signaling inhibitor.
E55. The method of E52, wherein the method further comprises identifying the subject as having high platelet levels prior to administration of the ActRII signaling inhibitor.
E56. A method of treating a cytopenia in a subject having myelofibrosis who has discontinued treatment with a JAK inhibitor, comprising administering to the subject an effective amount of an ActRII signaling inhibitor.
E57. A method of treating a subject having myelofibrosis who has discontinued treatment with a JAK inhibitor, comprising administering to the subject an effective amount of an ActRII signaling inhibitor.
E58. The method of E56 or E57, wherein the subject had relapsed disease following treatment with the JAK inhibitor.
E59. The method of E56 or E57, wherein the subject is refractory to treatment with the JAK inhibitor.
E60. The method of E56 or E57, wherein the subject is intolerant to treatment with a JAK inhibitor or no longer meets the risk/benefit ratio to continue treatment with the JAK inhibitor.
E61. A method of treating a cytopenia in a subject having myelofibrosis who is ineligible for treatment with a JAK inhibitor, comprising administering to the subject an effective amount of an ActRII signaling inhibitor.
E62. A method of treating a subject having myelofibrosis who is ineligible for treatment with a JAK inhibitor, comprising administering to the subject an effective amount of an ActRII signaling inhibitor.
E63. The method of any one of E56-E62, wherein the JAK inhibitor is ruxolitinib, fedratinib, or pacritinib.
E64. The method of any one of E8, E39-E43, and E45-E63, wherein the myelofibrosis is PMF, post-ET MF, or post-PV MF.
E65. The method of any one of E38-E55, E57-E60, and E62-E64, wherein the subject has a cytopenia.
E66. The method of any one of E8 and E36-E65, wherein the myelofibrosis is intermediate- or high-risk myelofibrosis.
E67. A method of treating a subject having a cytopenia associated with treatment with an antifungal agent, comprising administering to the subject an effective amount of an ActRII signaling inhibitor.
E68. The method of E67, wherein the antifungal agent is ketoconazole, terbinafine, fluconazole, micafungin, or caspofungin.
E69. A method of treating a subject having a cytopenia associated with immunosuppressant treatment, comprising administering to the subject an effective amount of an ActRII signaling inhibitor.
E70. The method of E69, wherein the immunosuppressant is azathioprine, methotrexate, or mycophenolate mofetil.
E71. A method of treating a subject having anemia associated with antibiotic treatment, comprising administering to the subject an effective amount of an ActRII signaling inhibitor.
E72. The method of E71, wherein the antibiotic is a cephalosporin or a penicillin.
E73. The method of any one of E1-E72, wherein the subject does not respond well to treatment with erythropoietin (EPO), is susceptible to the adverse effects of EPO, or does not respond well to treatment with an erythroid maturation agent.
E74. The method of any one of E1-E73, wherein the subject has previously been treated with an erythropoiesis stimulating agent (ESA).
E75. The method of any one of E1-E73, wherein the subject has not previously been treated with an erythropoiesis stimulating agent (ESA).
E76. The method of any one of E3-E75, wherein subject has not previously been treated with azacitidine, decitabine, lenalidomide, luspatercept, or sotatercept.
E77. The method of any one of E1-E76, wherein the subject has a low transfusion burden.
E78. The method of E77, wherein the subject has received 1-3 units of RBCs (1-3 RBC transfusions) within eight weeks prior to starting treatment with an ActRII signaling inhibitor.
E79. The method of E77, wherein the subject has received 0 units of RBCs (0 RBC transfusions) within eight weeks prior to starting treatment with an ActRII signaling inhibitor (i.e., the subject is a non-transfused subject).
E80. The method of any one of E1-E76, wherein the subject has a high transfusion burden.
E81. The method of any one of E1, E2, and E5-E80, wherein the subject has an erythropoietin level greater than 100 mlU/mL.
E82. The method of any one of E1, E2 and E5-E80, wherein the subject has an erythropoietin level less than or equal to 100 mlU/mL.
E83. The method of any one of E1-E78 and E80-E82, wherein the method reduces the subject's transfusion burden.
E84. The method of any one of E1-E78 and E80-E83, wherein the method promotes transfusion independence (e.g., transfusion independence for at least 8 weeks, 10 weeks, 12 weeks, 14 weeks, 16 weeks, 20 weeks, 24 weeks, 26 weeks, 1 year, 2 years or more during treatment with an ActRII signaling inhibitor compared to pretreatment transfusion data from the 8 weeks directly preceding treatment).
E85. The method of any one of E1-E79 and E81-E84, wherein the method leads to an increase in hemoglobin of ≥1.5 g/dL (e.g., an increase in hemoglobin of ≥1.5 g/dl for at least 2 weeks, 4 weeks, 6 weeks, 8 weeks, 10 weeks, 12 weeks, 14 weeks, 16 weeks, 20 weeks, 24 weeks, 26 weeks, 1 year, 2 years or more during treatment with an ActRII signaling inhibitor compared to baseline or pretreatment measurements).
E86. The method of any one of E1-E77 and E81-E85, wherein the method leads to a reduction of ≥4 U or ≥50% units transfused during a treatment period (e.g., a reduction of ≥4 U or ≥50% units transfused during a treatment period of 2 weeks, 4 weeks, 6 weeks, 8 weeks, 10 weeks, 12 weeks, 14 weeks, 16 weeks, 20 weeks, 24 weeks, 26 weeks, 1 year, 2 years or more with an ActRII signaling inhibitor compared to baseline in the 8 weeks preceding treatment).
E87. The method of any one of E1-E78 and E80-E86, wherein the subject achieves transfusion independence for at least eight weeks or twelve weeks during treatment (e.g., compared to pretreatment transfusion data from the 8 weeks or 12 weeks directly preceding treatment).
E88. The method of any one of E1-E87, wherein the cytopenia is anemia.
E89. The method of any one of E1-E88, wherein the subject is identified as having anemia prior to administration of the ActRII signaling inhibitor.
E90. The method of any one of E1-E88, wherein the method further comprises identifying the subject as having anemia prior to administration of the ActRII signaling inhibitor.
E91. The method of any one of E1-E90, wherein the method further comprises evaluating red blood cell count, hemoglobin levels, reticulocyte count, or hematocrit levels after administration of the ActRII signaling inhibitor.
E92. The method of any one of E1-E91, wherein the cytopenia is thrombocytopenia.
E93. The method of any one of E1-E92, wherein the subject is identified as having thrombocytopenia prior to administration of the ActRII signaling inhibitor.
E94. The method of any one of E1-E92, wherein the method further comprises identifying the subject as having thrombocytopenia prior to administration of the ActRII signaling inhibitor.
E95. The method of any one of E1-E94, wherein the method further comprises evaluating platelet levels after administration of the ActRII signaling inhibitor.
E96. The method of any one of E1-E95, wherein the cytopenia is neutropenia.
E97. The method of any one of E1-E96, wherein the subject is identified as having neutropenia prior to administration of the ActRII signaling inhibitor.
E98. The method of any one of E1-E96, wherein the method further comprises identifying the subject as having neutropenia prior to administration of the ActRII signaling inhibitor.
E99. The method of any one of E1-E98, wherein the method further comprises evaluating neutrophil levels after administration of the ActRII signaling inhibitor.
E100. The method of any one of E1-E99, wherein the method further comprises performing a complete blood count (CBC) after administration of the ActRII signaling inhibitor (e.g., 12 hours, 24 hours, 1, 2, 3, 4, 5, 6, or 7 days, 1, 2, 3, 4, 5, 6, 7, 8, 9, 10, 11, or 12 weeks, or 1, 2, 3, 4, 5, or 6 months or more after treatment initiation).
E101. The method of any one of E1-E100, wherein the method further comprises performing a CBC before administration of the ActRII signaling inhibitor.
E102. A method of improving hematopoietic stem cell engraftment in a subject in need thereof, comprising administering to the subject an effective amount of an ActRII signaling inhibitor prior to hematopoietic stem cell transplantation (e.g., prior to engraftment).
E103. A method of treating a subject having thrombocythemia, comprising administering to the subject an effective amount of an ActRII signaling inhibitor.
E104. A method of treating a subject having polycythemia vera, comprising administering to the subject an effective amount of ActRII signaling inhibitor.
E105. A method of reducing platelets in a subject in need thereof, comprising administering to the subject an effective amount of ActRII signaling inhibitor.
E106. The method of E105, wherein the subject has thrombocythemia or polycythemia vera, or requires phlebotomy due to excess red blood cells.
E107. The method of any one of E103-E106, wherein the subject is identified as having elevated platelet levels prior to administration of the ActRII signaling inhibitor.
E108. The method of any one of E1-E107, wherein the method reduces or inhibits the binding of activin A, activin B, and/or myostatin to their receptors (e.g., their endogenous receptors).
E109. The method of any one of E1-E108, wherein the ActRII signaling inhibitor is administered in an amount sufficient to increase red blood cell levels, increase hemoglobin levels, increase red blood cell production, increase red blood cell count, increase hematocrit, reduce transfusion burden, promote transfusion independence, increase mean corpuscular volume, increase mean corpuscular hemoglobin, increase reticulocyte cell hemoglobin, increase erythropoietin levels, increase thrombopoietin levels, increase the maturation and/or differentiation of erythroid progenitors (e.g., early- and/or late-stage erythroid progenitors), increase late-stage erythroid precursor maturation, recruit early-stage progenitors into the erythroid lineage, increase reticulocytes, increase proerythroblast numbers, reduce the accumulation of red blood cell progenitor cells, increase the number of early-stage erythroid precursors and/or progenitors, promote the progression of erythroid precursors and/or progenitors through erythropoiesis, treat anemia, increase platelet levels, increase platelet volume, increase immature platelet fraction, increase proplatelets, increase platelet production, increase platelet count, increase or induce megakaryocyte differentiation and/or maturation, increase megakaryocyte progenitor renewal, reduce the accumulation of platelet progenitor cells, improve blood clotting, reduce bleeding events, reduce bleeding in the skin, treat thrombocytopenia, increase neutrophil levels, increase neutrophil production, increase neutrophil count, increase or induce the differentiation and/or maturation of progenitor cells into neutrophils, treat neutropenia, reduce susceptibility to infection, affect myostatin, activin A, activin B, and/or BMP9 signaling in the subject, or reduce or inhibit the binding of activin A, activin B, and/or myostatin to their receptors (e.g., their endogenous receptors).
E110. The method of any one of E7, E8, and E31-E101, wherein the ActRII signaling inhibitor is administered in an amount sufficient to reduce spleen volume.
E111. The method of any one of E8 and E36-E101, wherein the ActRII signaling inhibitor is administered in an amount sufficient to reduce bone marrow fibrosis, reduce osteosclerosis, improve bone marrow fibrosis grade, or reduce high platelet levels.
E112. The method of any one of E1-E111, wherein the method does not cause a vascular complication in the subject.
E113. The method of E112, wherein the method does not increase vascular permeability or leakage.
E114. The method of any one of E1-E113, wherein the subject is a human.
E115. The method of any one of E1-E114, wherein the ActRII signaling inhibitor is an activin A antibody or an antigen binding fragment thereof.
E116. The method of E115, wherein the activin A antibody is garetosmab.
E117. The method of E115, wherein the activin A antibody or an antigen binding fragment thereof has a heavy chain variable region (HCVR) sequence having at least 90% (e.g., at least 91%, 92%, 93%, 94%, 95%, 96%, 97%, 98%, 99%, or 100%) sequence identity to a HCVR sequence in Table 1 and a light chain variable region (LCVR) sequence having at least 90% (e.g., at least 91%, 92%, 93%, 94%, 95%, 96%, 97%, 98%, 99%, or 100%) sequence identity to a LCVR sequence in Table 1 (e.g., an HCVR sequence in Table 1 and an LCVR sequence in Table 1, such as an HCVR sequence and an LCVR sequence from the same row of Table 1).
E118. The method of E115 or E117, wherein the activin A antibody or an antigen binding fragment thereof has a light chain CDR1, CDR2, and CDR3 and a heavy chain CDR1, CDR2, and CDR3 listed in Table 2 (e.g., a light chain CDR1, CDR2, and CDR3 sequence and a heavy chain CDR1, CDR2, and CDR3 sequence from the same row of Table 2).
E119. The method of any one of E1-E114, wherein the ActRII signaling inhibitor is a myostatin antibody or an antigen binding fragment thereof.
E120. The method of E119, wherein the myostatin antibody is domagrozumab, landogrozumab, trevogrumab, or SRK-015.
E121. The method of E119, wherein the myostatin antibody or an antigen binding fragment thereof has a HCVR sequence having at least 90% (e.g., at least 91%, 92%, 93%, 94%, 95%, 96%, 97%, 98%, 99%, or 100%) sequence identity to a HCVR sequence in Table 3 and a LCVR sequence having at least 90% (e.g., at least 91%, 92%, 93%, 94%, 95%, 96%, 97%, 98%, 99%, or 100%) sequence identity to a LCVR sequence in Table 3 (e.g., a HCVR sequence in Table 3 and a LCVR sequence in Table 3, such as an HCVR sequence and an LCVR sequence from the same row of Table 3 or an HCVR sequence of any one of SEQ ID NOs: 448-476 and an LCVR sequence of any one of SEQ ID NOs: 477-486).
E122. The method of E119 or E121, wherein the myostatin antibody or an antigen binding fragment thereof has a light chain CDR1, CDR2, and CDR3 and a heavy chain CDR1, CDR2, and CDR3 listed in Table 4, Table 5, or Table 6 (e.g., a light chain CDR1, CDR2, and CDR3 sequence and a heavy chain CDR1, CDR2, and CDR3 sequence from the same row of Table 4).
E123. The method of any one of E119, E121, and E122, wherein the myostatin antibody or an antigen binding fragment thereof has a heavy chain and light chain sequence having at least 90% sequence identity (e.g., 90%, 91%, 92%, 93%, 94%, 95%, 95%, 97%, 98%, 99% or 100% sequence identity) to a heavy chain and light chain sequence provided in Table 7 (e.g., a heavy chain and light chain sequence from the same row of Table 7).
E124. The method of any one of E1-E114, wherein the ActRII signaling inhibitor is an ActRII antibody or an antigen binding fragment thereof.
E125. The method of E124, wherein the ActRII antibody is bimagrumab, CSJ089, CQI876, or CDD861.
E126. The method of E124, wherein the ActRII antibody or an antigen binding fragment thereof has a HCVR sequence having at least 90% (e.g., at least 91%, 92%, 93%, 94%, 95%, 96%, 97%, 98%, 99%, or 100%) sequence identity to a HCVR sequence in Table 8 and a LCVR sequence having at least 90% (e.g., at least 91%, 92%, 93%, 94%, 95%, 96%, 97%, 98%, 99%, or 100%) sequence identity to a LCVR sequence in Table 8 (e.g., a HCVR sequence in Table 8 and a LCVR sequence in Table 8, such as an HCVR sequence and an LCVR sequence from the same row of Table 8).
E127. The method of E124 or E126, wherein the ActRII antibody or an antigen binding fragment thereof has a light chain CDR1, CDR2, and CDR3 and a heavy chain CDR1, CDR2, and CDR3 listed in Table 9 (e.g., a light chain CDR1, CDR2, and CDR3 sequence and a heavy chain CDR1, CDR2, and CDR3 sequence from the same row of Table 9).
E128. The method of any one of E124, E126, and E127, wherein the ActRII antibody or an antigen binding fragment thereof has a heavy chain and light chain sequence having at least 90% sequence identity (e.g., 90%, 91%, 92%, 93%, 94%, 95%, 95%, 97%, 98%, 99% or 100% sequence identity) to a heavy chain and light chain sequence provided in Table 10 (e.g., a heavy chain and light chain sequence from the same row of Table 10).
E129. The method of any one of E1-E114, wherein the ActRII signaling inhibitor is an ActRII ligand trap.
E130. The method of E129, wherein the ActRII ligand trap is an ActRIIA ligand trap.
E131. The method of E130, wherein the ActRIIA ligand trap is a composition of Table 18 (e.g., a polypeptide, nucleic acid molecule, vector, or pharmaceutical composition of Table 18).
E132. The method of E130, wherein the ActRIIA ligand trap comprises an extracellular portion of wild-type ActRIIA (e.g., SEQ ID NO: 73 or SEQ ID NO: 729).
E133. The method of E130, wherein the ActRIIA ligand trap is sotatercept.
E134. The method of E129, wherein the ActRII ligand trap is an ActRIIB ligand trap.
E135. The method of E134, wherein the ActRIIB ligand trap is BIIB110, ALG-802, luspatercept, ramatercept, or ACE-2494.
E136. The method of E134, wherein the ActRIIB ligand trap comprises an extracellular portion of wild-type ActRIIB (e.g., SEQ ID NO: 74 or a portion thereof).
E137. The method of E134, wherein the ActRIIB ligand trap is a composition of Table 19 (e.g., a polypeptide, nucleic acid molecule, vector, or pharmaceutical composition of Table 19).
E138. The method of E134, wherein the ActRIIB ligand trap comprises the sequence of any one of SEQ ID NOs: 745-750 (e.g., the sequence of any one of SEQ ID NOs: 745-750 fused to a moiety, such as an Fc domain or an Fc domain monomer, by way of a linker).
E139. The method of E129, wherein the ActRII ligand trap is an ActRII chimera ligand trap.
E140. The method of E139, wherein the ActRII chimera ligand trap is a composition of Table 20 or Table 21 (e.g., a polypeptide, nucleic acid molecule, vector, or pharmaceutical composition of Table 20 or Table 21).
E141. The method of any one of E1-E114, wherein the ActRII signaling inhibitor is an activin B antibody or an antigen binding fragment thereof.
E142. The method of E141, wherein the activin B antibody or an antigen binding fragment thereof has a HCVR having at least 90% (e.g., at least 91%, 92%, 93%, 94%, 95%, 96%, 97%, 98%, 99%, or 100%) sequence identity to SEQ ID NO: 494 and a LCVR having at least 90% (e.g., at least 91%, 92%, 93%, 94%, 95%, 96%, 97%, 98%, 99%, or 100%) sequence identity to SEQ ID NO: 495.
E143. The method of any one of E1-E114, wherein the ActRII signaling inhibitor is a GDF-11 antibody or an antigen binding fragment thereof.

### Definitions

To facilitate the understanding of this invention, a number of terms are defined below. Terms defined herein have meanings as commonly understood by a person of ordinary skill in the areas relevant to the invention. Terms such as “a”, “an,” and “the” are not intended to refer to only a singular entity but include the general class of which a specific example may be used for illustration. The terminology herein is used to describe specific embodiments of the invention, but their usage does not limit the invention, except as outlined in the claims.

As used herein, the term “about” refers to a value that is within 10% above or below the value being described.

As used herein, any values provided in a range of values include both the upper and lower bounds, and any values contained within the upper and lower bounds.

As used herein, “administration” refers to providing or giving a subject a therapeutic agent (e.g., an ActRII signaling inhibitor described herein), by any effective route. Exemplary routes of administration are described herein below.

The term “antibody” is used in the broadest sense and specifically covers intact monoclonal antibodies, polyclonal antibodies, multispecific antibodies (e.g., bispecific antibodies) formed from at least two intact antibodies, and antibody fragments so long as they exhibit the desired biological activity.

“Antibody fragments” include a portion of an intact antibody, preferably the antigen binding or variable region of the intact antibody. Examples of antibody fragments include Fab, Fab′, F(ab′)2, and Fv fragments; diabodies; linear antibodies (Zapata et al. Protein Eng. 8(10):1057-1062 (1995)); single-chain antibody molecules; and multispecific antibodies formed from antibody fragments.

As used herein, the term “extracellular activin receptor type IIA (ActRIIA) variant” refers to a peptide including a soluble, extracellular portion of the single transmembrane receptor, ActRIIA, that has at least one amino acid substitution relative to a wild-type extracellular ActRIIA (e.g., bold portion of the sequence of SEQ ID NO: 75 shown below). The sequence of the wild-type, human ActRIIA precursor protein is shown below (SEQ ID NO: 75), in which the signal peptide is italicized and the extracellular portion is bold.

Wild-type, human ActRIIA precursor protein
(SEQ ID NO: 75):
MGAAAKLAFAVFLISCSSGAILGRSETQECLFFNANWEKDRTNQTG
VEPCYGDKDKRRHCFATWKNISGSIEIVKQGCWLDDINCYDRTDC
VEKKDSPEVYFCCCEGNMCNEKFSYFPEMEVTQPTSNPVTPKPPY
YNILLYSLVPLMLIAGIVICAFWVYRHHKMAYPPVLVPTQDPGPP
PPSPLLGLKPLQLLEVKARGRFGCVWKAQLLNEYVAVKIFPIQDK
QSWQNEYEVYSLPGMKHENILQFIGAEKRGTSVDVDLWLITAFHE
KGSLSDFLKANVVSWNELCHIAETMARGLAYLHEDIPGLKDGHKP
AISHRDIKSKNVLLKNNLTACIADFGLALKFEAGKSAGDTHGQVG
TRRYMAPEVLEGAINFQRDAFLRIDMYAMGLVLWELASRCTAADG
PVDEYMLPFEEEIGQHPSLEDMQEVVVHKKKRPVLRDYWQKHAGM
AMLCETIEECWDHDAEARLSAGCVGERITQMQRLTNIITTEDIVT
VVTMVTNVDFPPKESSL

An extracellular ActRIIA variant may have a sequence of any one of SEQ ID NOs: 1-72. In particular embodiments, an extracellular ActRIIA variant has a sequence of any one of SEQ ID NOs: 6-72 (Table 12). In some embodiments, an extracellular ActRIIA variant may have at least 85% (e.g., at least 85%, 87%, 90%, 92%, 95%, 97%, or greater) amino acid sequence identity to the sequence of a wild-type extracellular ActRIIA (SEQ ID NO: 73).

As used herein, the term “linker” refers to a linkage between two elements, e.g., peptides or protein domains. An ActRII ligand trap described herein may include an extracellular portion of ActRIIA, ActRIIB, a variant thereof, or a chimera thereof (e.g., an extracellular ActRIIA variant having a sequence of any one of SEQ ID NOs: 1-72 (e.g., SEQ ID NOs: 6-72)) fused to a moiety. The moiety may increase stability or improve pharmacokinetic properties of the polypeptide. The moiety (e.g., Fc domain monomer, Fc domain, an albumin-binding peptide, a fibronectin domain, or a human serum albumin) may be fused to the polypeptide by way of a linker. A linker can be a covalent bond or a spacer. The term “bond” refers to a chemical bond, e.g., an amide bond or a disulfide bond, or any kind of bond created from a chemical reaction, e.g., chemical conjugation. The term “spacer” refers to a moiety (e.g., a polyethylene glycol (PEG) polymer) or an amino acid sequence (e.g., a 1-200 amino acid sequence) occurring between two elements, e.g., peptides or protein domains, to provide space and/or flexibility between the two elements. An amino acid spacer is part of the primary sequence of a polypeptide (e.g., fused to the spaced peptides via the polypeptide backbone). The formation of disulfide bonds, e.g., between two hinge regions that form an Fc domain, is not considered a linker.

As used herein, the term “Fc domain” refers to a dimer of two Fc domain monomers. An Fc domain has at least 80% sequence identity (e.g., at least 85%, 90%, 95%, 97%, or 100% sequence identity) to a human Fc domain that includes at least a C_{H}2 domain and a C_{H}3 domain. An Fc domain monomer includes second and third antibody constant domains (C_{H}2 and C_{H}3). In some embodiments, the Fc domain monomer also includes a hinge domain. An Fc domain does not include any portion of an immunoglobulin that is capable of acting as an antigen-recognition region, e.g., a variable domain or a complementarity determining region (CDR). In the wild-type Fc domain, the two Fc domain monomers dimerize by the interaction between the two C_{H}3 antibody constant domains, as well as one or more disulfide bonds that form between the hinge domains of the two dimerizing Fc domain monomers. In some embodiments, an Fc domain may be mutated to lack effector functions, typical of a “dead Fc domain.” In certain embodiments, each of the Fc domain monomers in an Fc domain includes amino acid substitutions in the C_{H}2 antibody constant domain to reduce the interaction or binding between the Fc domain and an Fcγ receptor. In some embodiments, the Fc domain contains one or more amino acid substitutions that reduce or inhibit Fc domain dimerization. An Fc domain can be any immunoglobulin antibody isotype, including IgG, IgE, IgM, IgA, or IgD. Additionally, an Fc domain can be an IgG subtype (e.g., IgG1, IgG2a, IgG2b, IgG3, or IgG4). The Fc domain can also be a non-naturally occurring Fc domain, e.g., a recombinant Fc domain.

As used herein, the term “albumin-binding peptide” refers to an amino acid sequence of 12 to 16 amino acids that has affinity for and functions to bind serum albumin. An albumin-binding peptide can be of different origins, e.g., human, mouse, or rat. In some embodiments, an albumin-binding peptide has the sequence DICLPRWGCLW (SEQ ID NO: 83).

As used herein, the term “fibronectin domain” refers to a high molecular weight glycoprotein of the extracellular matrix, or a fragment thereof, that binds to, e.g., membrane-spanning receptor proteins such as integrins and extracellular matrix components such as collagens and fibrins. In some embodiments, a fibronectin domain is a fibronectin type III domain (SEQ ID NO: 82) having amino acids 610-702 of the sequence of UniProt ID NO: P02751. In other embodiments, a fibronectin domain is an adnectin protein.

As used herein, the term “human serum albumin” refers to the albumin protein present in human blood plasma. Human serum albumin is the most abundant protein in the blood. It constitutes about half of the blood serum protein. In some embodiments, a human serum albumin has the sequence of UniProt ID NO: P02768 (SEQ ID NO: 81).

As used herein, the term “endogenous” describes a molecule (e.g., a polypeptide, nucleic acid, or cofactor) that is found naturally in a particular organism (e.g., a human) or in a particular location within an organism (e.g., an organ, a tissue, or a cell, such as a human cell, e.g., a human red blood cell, platelet, neutrophil, or muscle cell).

As used herein, the term “fused” is used to describe the combination or attachment of two or more elements, components, or protein domains, e.g., peptides or polypeptides, by means including chemical conjugation, recombinant means, and chemical bonds, e.g., amide bonds. For example, two single peptides in tandem series can be fused to form one contiguous protein structure, e.g., a polypeptide, through chemical conjugation, a chemical bond, a peptide linker, or any other means of covalent linkage. In some embodiments of an ActRII ligand trap described herein, an extracellular portion of ActRIIA, ActRIIB, a variant thereof, or a chimera thereof (e.g., an extracellular ActRIIA variant having the sequence of any one of SEQ ID NOs: 1-72 (e.g., SEQ ID NOs: 6-72)) may be fused in tandem series to the N- or C-terminus of a moiety (e.g., Fc domain monomer (e.g., the sequence of SEQ ID NO: 97), an Fc domain (e.g., the sequence of SEQ ID NO: 84 or SEQ ID NO: 79), an albumin-binding peptide (e.g., the sequence of SEQ ID NO: 83), a fibronectin domain (e.g., the sequence of SEQ ID NO: 82), or a human serum albumin (e.g., the sequence of SEQ ID NO: 81)) by way of a linker. For example, an extracellular ActRIIA variant is fused to a moiety (e.g., an Fc domain monomer, an Fc domain, an albumin-binding peptide, a fibronectin domain, or a human serum albumin) by way of a peptide linker, in which the N-terminus of the peptide linker is fused to the C-terminus of the extracellular ActRIIA variant through a chemical bond, e.g., a peptide bond, and the C-terminus of the peptide linker is fused to the N-terminus of the moiety (e.g., Fc domain monomer, Fc domain, albumin-binding peptide, fibronectin domain, or human serum albumin) through a chemical bond, e.g., a peptide bond.

As used herein, the term “C-terminal extension” refers to the addition of one or more amino acids to the C-terminus of a polypeptide including an extracellular ActRIIA variant (e.g., an extracellular ActRIIA variant having the sequence of any one of SEQ ID NOs: 1-70 (e.g., SEQ ID NOs: 6-70)). The C-terminal extension can be one or more amino acids, such as 1-6 amino acids (e.g., 1, 2, 3, 4, 5, 6 or more amino acids). The C-terminal extension may include amino acids from the corresponding position of wild-type ActRIIA. Exemplary C-terminal extensions are the amino acid sequence NP (a two amino acid C-terminal extension) and the amino acid sequence NPVTPK (SEQ ID NO: 78) (a six amino acid C-terminal extension). Any amino acid sequence that does not disrupt the activity of the polypeptide can be used. SEQ ID NO: 71, which is the sequence of SEQ ID NO: 69 with a C-terminal extension of NP, and SEQ ID NO: 72, which is the sequence of SEQ ID NO: 69 with a C-terminal extension of NPVTPK (SEQ ID NO: 78), represent two of the possible ways that a polypeptide of the invention can be modified to include a C-terminal extension.

As used herein, the term “percent (%) identity” refers to the percentage of amino acid (or nucleic acid) residues of a candidate sequence, e.g., an extracellular ActRIIA variant, that are identical to the amino acid (or nucleic acid) residues of a reference sequence, e.g., a wild-type extracellular ActRIIA (e.g., SEQ ID NO: 73), after aligning the sequences and introducing gaps, if necessary, to achieve the maximum percent identity (i.e., gaps can be introduced in one or both of the candidate and reference sequences for optimal alignment and non-homologous sequences can be disregarded for comparison purposes). Alignment for purposes of determining percent identity can be achieved in various ways that are within the skill in the art, for instance, using publicly available computer software such as BLAST, ALIGN, or Megalign (DNASTAR) software. Those skilled in the art can determine appropriate parameters for measuring alignment, including any algorithms needed to achieve maximal alignment over the full length of the sequences being compared. In some embodiments, the percent amino acid (or nucleic acid) sequence identity of a given candidate sequence to, with, or against a given reference sequence (which can alternatively be phrased as a given candidate sequence that has or includes a certain percent amino acid (or nucleic acid) sequence identity to, with, or against a given reference sequence) is calculated as follows:

$$100 \times ( fraction of A / B )$$

where A is the number of amino acid (or nucleic acid) residues scored as identical in the alignment of the candidate sequence and the reference sequence, and where B is the total number of amino acid (or nucleic acid) residues in the reference sequence. In some embodiments where the length of the candidate sequence does not equal to the length of the reference sequence, the percent amino acid (or nucleic acid) sequence identity of the candidate sequence to the reference sequence would not equal to the percent amino acid (or nucleic acid) sequence identity of the reference sequence to the candidate sequence.

In particular embodiments, a reference sequence aligned for comparison with a candidate sequence may show that the candidate sequence exhibits from 50% to 100% identity across the full length of the candidate sequence or a selected portion of contiguous amino acid (or nucleic acid) residues of the candidate sequence. The length of the candidate sequence aligned for comparison purpose is at least 30%, e.g., at least 40%, e.g., at least 50%, 60%, 70%, 80%, 90%, or 100% of the length of the reference sequence. When a position in the candidate sequence is occupied by the same amino acid (or nucleic acid) residue as the corresponding position in the reference sequence, then the molecules are identical at that position.

As used herein, the term “serum half-life” refers to, in the context of administering a therapeutic protein to a subject, the time required for plasma concentration of the protein in the subject to be reduced by half. The protein can be redistributed or cleared from the bloodstream, or degraded, e.g., by proteolysis. Serum half-life comparisons can be made by comparing the serum half-life of Fc fusion proteins.

As used herein, the term “affinity” or “binding affinity” refers to the strength of the binding interaction between two molecules. Generally, binding affinity refers to the strength of the sum total of non-covalent interactions between a molecule and its binding partner, such as an extracellular ActRIIA variant and BMP9 or activin A. Unless indicated otherwise, binding affinity refers to intrinsic binding affinity, which reflects a 1:1 interaction between members of a binding pair. The binding affinity between two molecules is commonly described by the dissociation constant (K_{D}) or the affinity constant (K_{A}). Two molecules that have low binding affinity for each other generally bind slowly, tend to dissociate easily, and exhibit a large K_{D}. Two molecules that have high affinity for each other generally bind readily, tend to remain bound longer, and exhibit a small K_{D}. The Ko of two interacting molecules may be determined using methods and techniques well known in the art, e.g., surface plasmon resonance. K_{D} is calculated as the ratio of K_{off}/Kₒₙ.

As used herein, the phrase “affecting myostatin, activin A, activin B, and/or BMP9 signaling” means changing the binding of myostatin, activin A, activin B, and/or BMP9 to their receptors, e.g., ActRIIA, ActRIIB, and/or BMPRII (e.g., ActRIIA, e.g., endogenous ActRIIA). In some embodiments, a polypeptide including an extracellular ActRIIA variant described herein reduces or inhibits the binding of myostatin, activin A, activin B, and/or BMP9 to their receptors, e.g., ActRIIA, ActRIIB, and/or BMPRII (e.g., ActRIIA, e.g., endogenous ActRIIA).

As used herein, the terms “increasing” and “decreasing” refer to modulating resulting in, respectively, greater or lesser amounts, of function, expression, or activity of a metric relative to a reference. For example, subsequent to administration of a polypeptide of the invention including an extracellular ActRIIA variant in a method described herein, the amount of a marker of a metric (e.g., hemoglobin levels, red blood cell count, hematocrit, reticulocyte count, platelet count, or transfusion burden) as described herein may be increased or decreased in a subject relative to the amount of the marker prior to administration. Generally, the metric is measured subsequent to administration at a time that the administration has had the recited effect, e.g., at least one week, one month, 3 months, or 6 months, after a treatment regimen has begun.

As used herein, the terms “increase red blood cell levels” and “promote red blood cell formation” refer to clinically observable metrics, such as hematocrit, red blood cell counts, and hemoglobin measurements, and are intended to be neutral as to the mechanism by which such changes occur. The terms “red blood cell formation” and “red blood cell production” refer to the generation of red blood cells, such as the process of erythropoiesis in which red blood cells are produced in the bone marrow.

As used herein, the term “anemia” refers to any abnormality in hemoglobin or red blood cells that leads to reduced oxygen levels in the blood. Anemia can be associated with abnormal production, processing, or performance of erythrocytes and/or hemoglobin. The term anemia refers to any reduction in the number of red blood cells and/or level of hemoglobin in blood relative to normal blood levels. For example, a subject having a hemoglobin level ≤10 g/dL or receiving red blood cell (RBC) transfusions can be identified as having anemia.

As used herein, the terms “increase platelet levels” and “promote platelet formation” refer to clinically observable metrics, such as platelet counts, and are intended to be neutral as to the mechanism by which such changes occur. The terms “platelet formation” and “platelet production” refer to the generation of platelets, such as the process in which platelets are produced from megakaryocytes.

As used herein, the terms “increase neutrophil levels” and “promote neutrophil formation” refer to clinically observable metrics, such as neutrophil counts, and are intended to be neutral as to the mechanism by which such changes occur. The terms “neutrophil formation” and “neutrophil production” refer to the generation of neutrophils such as the process in which neutrophils are produced in the bone marrow.

As used herein, the term “thrombocytopenia” refers to a condition in which the blood contains a lower than normal number of platelets, which may be due to a deficiency in platelet production, accumulation of platelets within an enlarged spleen, or the destruction of platelets. Normal blood platelet levels range from about 150,000 to 450,000 per microliter blood in humans. A platelet count of less than 150,000 platelets per microliter is lower than normal. Bleeding can occur after a relatively minor injury if the platelet count falls below 50,000 platelets per microliter of blood, and serious bleeding may occur without any recognized injury if the platelet count falls below 10,000 to 20,000 platelets per microliter of blood.

As used herein, the term “neutropenia” refers to a condition in which the blood contains an abnormally low number of neutrophils. The typical lower limit of the neutrophil count is about 1500 cells per microliter of blood. Below this level, the risk of infection increases. Neutropenia severity is classified as: mild (1000 to 1500 neutrophils per microliter of blood), moderate (500 to 1000 neutrophils per microliter of blood), and severe (below 500 neutrophils per microliter of blood). Neutropenia has many causes, but they typically fall into two main categories: destruction or depletion of neutrophils faster than the bone marrow can produce new neutrophils, or reduced production of neutrophils in the bone marrow.

As used herein, the term “low transfusion burden” refers to a condition of a subject that has received less than four units of red blood cells (RBCs) within eight weeks (e.g., 3, 2, 1, or 0 units of RBCs within eight weeks) prior to treatment with an ActRIIA variant described herein. A subject with a low transfusion burden can be identified as having anemia based on measurements of mean hemoglobin concentration. A subject with a low transfusion burden and a mean hemoglobin concentration of less than 10.0 g/dL of two measurements performed at least one week apart prior to treatment with an ActRIIA variant described herein (e.g., one measurement performed within one day prior to treatment and the other performed 7-28 days prior, not influenced by RBC transfusion within seven days of measurement) is defined as having anemia. In some embodiments, a subject with a low transfusion burden receives 1-3 units of RBCs (1-3 RBC transfusions) within eight weeks prior to treatment with an ActRIIA variant described herein. In some embodiments, a subject with a low transfusion burden does not receive any units of RBCs (0 RBC transfusions) within eight weeks prior to treatment with an ActRIIA variant described herein. A subject with a low transfusion burden who does not receive any units of RBCs (0 RBC transfusions) within eight weeks prior to treatment with an ActRIIA variant described herein can also be referred to as a “non-transfused” subject.

As used herein, the term “high transfusion burden” refers to a condition of a subject requiring greater than or equal to four units of RBCs (e.g., 4, 5, 6, 7, 8, or more units) within eight weeks prior to treatment with an ActRIIA variant described herein. A subject with a high transfusion burden can be identified as having anemia based on measurements of mean hemoglobin concentration. A subject with a high transfusion burden and a mean hemoglobin concentration of less than or equal to 9.0 g/dl is defined as having anemia.

As used herein, the term “ineffective hematopoiesis” refers to the failure to produce fully mature hematopoietic cells (e.g., the failure to produce red blood cells, platelets, and neutrophils). Ineffective hematopoiesis may be due to single or multiple defects, such as abnormal proliferation and/or differentiation of progenitor cells (e.g., an excessive production of progenitors that are unable to complete differentiation), that can lead to a hyperproliferation or a shortage of progenitor cells.

As used herein, the terms “erythropoiesis stimulating agent” and “ESA” refer to a class of drugs that act on the proliferation stage of red blood cell development by expanding the pool of early-stage progenitor cells. Examples of erythropoiesis-stimulating agents are epoetin alfa and darbepoetin alfa. As used herein, the term “vascular complication” refers to a vascular disorder or any damage to the blood vessels, such as damage to the blood vessel walls. Damage to the blood vessel walls may cause an increase in vascular permeability or leakage. The term “vascular permeability or leakage” refers to the capacity of the blood vessel walls to allow the flow of small molecules, proteins, and cells in and out of blood vessels. An increase in vascular permeability or leakage may be caused by an increase in the gaps (e.g., an increase in the size and/or number of the gaps) between endothelial cells that line the blood vessel walls and/or thinning of the blood vessel walls.

As used herein, the term “polypeptide” describes a single polymer in which the monomers are amino acid residues which are covalently conjugated together through amide bonds. A polypeptide is intended to encompass any amino acid sequence, either naturally occurring, recombinant, or synthetically produced.

As used herein, the term “homodimer” refers to a molecular construct formed by two identical macromolecules, such as proteins or nucleic acids. The two identical monomers may form a homodimer by covalent bonds or non-covalent bonds. For example, an Fc domain may be a homodimer of two Fc domain monomers if the two Fc domain monomers contain the same sequence. In another example, a polypeptide described herein including an extracellular ActRIIA variant fused to an Fc domain monomer may form a homodimer through the interaction of two Fc domain monomers, which form an Fc domain in the homodimer.

As used herein, the term “heterodimer” refers to a molecular construct formed by two different macromolecules, such as proteins or nucleic acids. The two monomers may form a heterodimer by covalent bonds or non-covalent bonds. For example, a polypeptide described herein including an extracellular ActRIIA variant fused to an Fc domain monomer may form a heterodimer through the interaction of two Fc domain monomers, each fused to a different ActRIIA variant, which form an Fc domain in the heterodimer.

As used herein, the term “host cell” refers to a vehicle that includes the necessary cellular components, e.g., organelles, needed to express proteins from their corresponding nucleic acids. The nucleic acids are typically included in nucleic acid vectors that can be introduced into the host cell by conventional techniques known in the art (transformation, transfection, electroporation, calcium phosphate precipitation, direct microinjection, etc.). A host cell may be a prokaryotic cell, e.g., a bacterial cell, or a eukaryotic cell, e.g., a mammalian cell (e.g., a CHO cell or a HEK293 cell).

As used herein, the term “therapeutically effective amount” refers an amount of a polypeptide, nucleic acid, or vector of the invention or a pharmaceutical composition containing a polypeptide, nucleic acid, or vector of the invention effective in achieving the desired therapeutic effect in treating a patient having a disease or condition, such as a cytopenia (e.g., anemia, thrombocytopenia, or neutropenia) associated with a myelodysplastic syndrome or myelofibrosis. In particular, the therapeutically effective amount of the polypeptide, nucleic acid, or vector avoids adverse side effects.

As used herein, the term “pharmaceutical composition” refers to a medicinal or pharmaceutical formulation that includes an active ingredient as well as excipients and diluents to enable the active ingredient suitable for the method of administration. The pharmaceutical composition of the present invention includes pharmaceutically acceptable components that are compatible with the polypeptide, nucleic acid, or vector. The pharmaceutical composition may be in tablet or capsule form for oral administration or in aqueous form for intravenous or subcutaneous administration.

As used herein, the term “pharmaceutically acceptable carrier or excipient” refers to an excipient or diluent in a pharmaceutical composition. The pharmaceutically acceptable carrier must be compatible with the other ingredients of the formulation and not deleterious to the recipient. In the present invention, the pharmaceutically acceptable carrier or excipient must provide adequate pharmaceutical stability to a polypeptide described herein (e.g., an ActRII signaling inhibitor, such as an ActRII ligand trap including an extracellular ActRIIA variant), the nucleic acid molecule(s) encoding the polypeptide, or a vector containing such nucleic acid molecule(s). The nature of the carrier or excipient differs with the mode of administration. For example, for intravenous administration, an aqueous solution carrier is generally used; for oral administration, a solid carrier is preferred.

As used herein, the term “treating and/or preventing” refers to the treatment and/or prevention of a disease or condition, e.g., a cytopenia (e.g., anemia, thrombocytopenia, or neutropenia) associated with a myelodysplastic syndrome or myelofibrosis, using methods and compositions of the invention. Generally, treating a disease or condition, e.g., a cytopenia (e.g., anemia, thrombocytopenia, or neutropenia) associated with a myelodysplastic syndrome or myelofibrosis, occurs after a subject has developed the disease or condition. Preventing a disease or condition, e.g., a cytopenia (e.g., anemia, thrombocytopenia, or neutropenia) associated with a myelodysplastic syndrome or myelofibrosis, refers to steps or procedures taken when a subject is at risk of developing the disease or condition. The subject may show signs or mild symptoms that are judged by a physician to be indications or risk factors for developing the disease or condition, have another disease or condition associated with development of the disease or condition, be undergoing treatment that may cause the disease or condition, or have a family history or genetic predisposition of developing the disease or condition, but has not yet developed the disease or condition.

As used herein, the term “subject” refers to a mammal, e.g., preferably a human. Mammals include, but are not limited to, humans and domestic and farm animals, such as monkeys (e.g., a cynomolgus monkey), mice, dogs, cats, horses, and cows, etc.

### DESCRIPTION OF THE DRAWINGS

FIG. 1 is a sequence alignment showing the wild-type sequences of extracellular ActRIIA and ActRIIB and the sequences of exemplary ActRIIA variants.

FIG. 2 is a graph showing the effect of ActRIIA/B-hFc (SEQ ID NO: 80) on transfusion burden. Patients requiring transfusions at baseline (≥2 RBC units/8 weeks) that completed 8 weeks of treatment with ActRIIA/B-hFc were evaluated for transfusion reduction. Six patients required transfusion at baseline (2-10 RBC units over 8 weeks). As shown in FIG. 2, clinically meaningful reductions in transfusion burden as well as transfusion independence were observed with a Q4W dosing schedule after 8 weeks of treatment. *Baseline transfusion burden over 8 weeks, **Percent transfusion reduction over 8 weeks on treatment compared to baseline transfusion burden.

FIG. 3 is a series of graphs showing the effect of ActRIIA/B-mFc (SEQ ID NO: 69 fused to a mouse Fc domain by way of a linker) on thrombopoiesis. As shown in FIG. 3, ActRIIA/B-mFc increased circulating platelet numbers and bone marrow megakaryocyte progenitors within 12 hours post-dose. Data are represented as mean±SEM. Statistical analysis was performed using a Student T-Test; *p<0.05; ****p<0.0001.

FIG. 4 is a series of graphs showing that treatment with ActRIIA/B-mFc had a direct effect on megakaryocyte maturation. Treatment of eleven-week-old C57/B16 mice with ActRIIA/B-mFc (10 mg/kg via subcutaneous administration), led to an increase in the number of CD41+ megakaryocyte progenitors at 12 hours post-dose (left) and an increase in the number of polyploid megakaryocytes by 24 hours post-dose (right). Data are represented as mean±SEM.

FIGS. 5A-5C are a series of graphs showing that treatment with ActRIIA/B-mFc accelerated recovery of platelet numbers following platelet depletion compared to vehicle treatment. Eleven-week-old mice were treated with either anti-GP1bα (0.08 mg/kg, Efferet) or IgG control. At day 4 after treatment, the anti-GP1bα treated group was further divided to receive either vehicle or ActRIIA/B-mFc (7.5 mg/kg) treatment. Platelets were measured at the indicated time points post anti-GP1bα dosing. At day 10 post-treatment, mice were euthanized and bone marrow cells were harvested. As shown in FIG. 5A, mice treated with ActRIIA/B-mFc exhibited accelerated recovery of platelet numbers following platelet depletion compared to vehicle-treated mice in this mouse model of immune thrombocytopenia. Additionally, as shown in FIGS. 5B-5C, there was a 25% increase in the number of CD41+ megakaryocyte progenitors in the bone marrow of the ActRIIA/B-mFc-treated group compared to the vehicle-treated group and a higher 4N ploidy level at day 10 after platelet depletion. For platelet data, statistical analysis was performed with repeat measures mixed effect modeling. Individual comparisons shown are from a Tukey post-test. For CD41 data, statistical analysis was performed with 1-Way ANOVA and individual comparisons calculated using a Tukey post-test. *p<0.05; **p<0.01; ***p<0.001; ****p<0.0001.

FIG. 6 is a series of graphs showing that treatment with a single dose of ActRIIA/B-mFc resulted in increased circulating platelets for at least 85 days. Eleven-week-old C57BL/6 mice were treated with a single dose of either vehicle or ActRIIA/B-mFc (10 mg/kg) by subcutaneous administration. Separate cohorts of mice from both dosing groups were sampled for whole blood at study day 37, 51 and 85, and platelet counts were determined using a veterinary hematology analyzer (Heska Element HT5). Data are shown as mean±SEM. Statistical analysis was performed using a Student T-Test; *p≤0.05; **p≤0.01; *** p≤0.001; ****p≤0.0001.

FIG. 7 is a graph showing that ex-vivo treatment with ActRIIA/B-mFc reversed activin-mediated changes in megakaryocyte precursors. Bone marrow cells from 11-week-old C57Bl/6 mice were isolated and treated with Activin A (5 mg/kg), ActRIIA/B-mFc (10 mg/kg), or a combination of both for six days. Cells were harvested after six days and analyzed using flow cytometry (N=2). Error bars=SEM.

FIG. 8 is a graph showing that treatment with an anti-activin A antibody increased platelets in wild-type mice. Ten-week-old C57Bl/6 male mice were dosed with either TBS (vehicle), anti-activin A (5 mg/kg), or ActRIIA/B-mFc (10 mg/kg) via intraperitoneal administration. Twenty-four hours post-dose whole blood was sampled, and platelet counts were determined using a veterinary hematology analyzer (Hematrue). Data are represented as mean±SEM. Statistical analysis was performed using a one-way ANOVA, **p<0.01, ***p<0.001.

FIG. 9 is a series of graphs showing that platelet number and platelet volume were increased in the TPO^{high} model of myelofibrosis and that treatment with ActRIIA/B-mFc attenuated the expansion of platelets. Seven-week-old C57Bl/6 albino mice (B6(Cg)-Tyr, Jackson Laboratory) were teil-vein injected with 0.75 mg/kg thrombopoietin (TPO) expressing plasmid cloned into pLEV113 plasmid (Lake Pharma). The injection was done in a hydrodynamic approach in which 100 ml/kg volume was injected in a short period of time (6-10 seconds). On day 3 after TPO injection, mice were divided into 2 groups receiving either vehicle (TBS) or ActRIIA/B-mFc (7.5 mg/kg), twice weekly via IP injection. Mice were sacrificed on day 14 after TPO injection. Hematological parameters were analyzed by measured using the Heska Element HT5 veterinary hematology analyzer. N=10-12 mice/group. Results are presented as mean±SEM. Statistical analysis was performed by one-way ANOVA. ****=p<0.0001.

FIG. 10 is a series of graphs showing that ActRIIA/B-mFc improved TPO-induced anemia. The TPO^{high} model of myelofibrosis was anemic after 14 days of TPO expression and exhibited reduced red blood cells, hemoglobin, hematocrit, and mean corpuscular hemoglobin concentration. Treatment with ActRIIA/B-mFc was associated with a significant improvement in RBC metrics and appeared to reduce the development of anemia in this model. N=10-12 mice/group. Results are presented as mean±SEM. Statistical analysis was performed by one-way ANOVA. **=p<0.01; ***=p<0.001; ****=p<0.0001.

FIG. 11 is a graph showing that ActRIIA/B-mFc reduced TPO-induced splenic extra medullary hematopoiesis. In the TPO^{high} model of myelofibrosis, the expansion in megakaryocyte growth and proliferation reduces the capacity of bone marrow for hematopoiesis, inducing compensatory extra medullary hematopoiesis in the liver and spleen. These data show a significant reduction in splenomegaly in mice treated with ActRIIA/B-mFc, indicating reduced splenic extra medullary hematopoiesis, likely due to a reduced requirement for this compensatory process. N=10-12 mice/group. Results are presented as mean±SEM. Statistical analysis was performed by one-way ANOVA. *=p<0.05; ****=p<0.0001.

FIG. 12 is a series of graphs showing that ActRIIA/B-mFc reduced the TPO-mediated increase in white blood cells and lymphocytes. The TPO^{high} model of myelofibrosis exhibited an increase in white blood cells, neutrophils, and lymphocytes. Treatment with ActRIIA/B-mFc reduced the increase in white blood cells and lymphocytes in TPO^{high} mice. N=10-12 mice/group. Results are presented as mean±SEM. Statistical analysis was performed by one-way ANOVA. ***=p<0.001; ****=p<0.0001.

FIGS. 13A-13C are a series of graphs showing that treatment with ActRIIA/B-hFc resulted in a reduction in transfusion burden, increased erythropoiesis, and increased platelet counts in human subjects (n=5, transfused responders, TR). FIG. 13A shows observed maximum reductions in transfusion burden over 8 weeks. FIGS. 13B-13C show observed reticulocyte changes (FIG. 13B) and observed platelet changes (FIG. 13C).

FIG. 14 is a graph showing the treatment emergent adverse events with a frequency of ≥10% that occurred during treatment of Cohorts 1, 2, 3, and 4 with ActRIIA/B-hFc.

FIG. 15 is a graph showing the effect of treatment with ActRIIA/B-hFc on the maximum reduction in transfusions over eight weeks in subjects having a baseline transfusion requirement of ≥2 RBC units in Cohorts 1, 2, and 3.

FIGS. 16A-16D are a series of graphs showing that ActRIIA/B-hFc treatment demonstrated improvement in erythropoiesis and thrombopoiesis in both RS+ and non-RS MDS patients. Increases in reticulocytes, increases in serum soluble transferrin receptor (sTfR), and decreases in serum ferritin were observed in patients achieving HI-E or TI endpoints (FIGS. 16A-16C). Sustained increases in platelets were also observed in patients achieving HI-E or TI endpoints (FIG. 16D). Arrows indicate ActRIIA/B-hFc dose.

FIG. 17 is a graph showing the effect of treatment with ActRIIA/B-hFc on the maximum reduction in transfusions over eight weeks in subjects having a baseline transfusion requirement of ≥2 RBC units in Cohorts 1, 2, 3, and 4.

FIGS. 18A-18D are a series of graphs showing that ActRIIA/B-hFc treatment demonstrated improvement in erythropoiesis and thrombopoiesis in patients in Cohorts 1, 2, 3, and 4. Increases in reticulocytes, increases in serum soluble transferrin receptor (sTfR), and decreases in serum ferritin were observed in patients achieving HI-E or TI endpoints (FIGS. 18A-18C). Sustained increases in platelets were also observed in patients achieving HI-E or TI endpoints (FIG. 18D). Arrows indicate ActRIIA/B-hFc dose.

FIG. 19 is a graph showing the treatment emergent adverse events with a frequency of ≥10% that occurred during treatment of Cohorts 1-5 with ActRIIA/B-hFc.

FIG. 20 is a graph showing the effect of treatment with ActRIIA/B-hFc on the maximum reduction in transfusion burden over eight weeks in subjects having a baseline transfusion requirement of ≥2 RBC units in Cohorts 1-5. Treatment with ActRIIA/B-hFc resulted in HI-E and TI in transfusion dependent non-RS and RS+ MDS patients.

FIG. 21 is a graph showing that a sustained increase in platelets was observed in HTB patients achieving HI-E or TI with ActRIIA/B-hFc treatment. Arrows indicate ActRIIA/B-hFc dose.

FIG. 22 is a graph showing the effect of treatment with ActRIIA/B-hFc on reticulocytes in HTB patients. Arrows indicate ActRIIA/B-hFc dose.

FIG. 23 is a graph showing the effect of treatment with ActRIIA/B-hFc on soluble transferrin receptor (sTfR) and ferritin in HTB patients.

FIG. 24 is a series of graphs showing the effect of treatment with ActRIIA/B-hFc on markers of erythropoiesis during the first eight weeks of treatment. Dose-dependent increases were observed in reticulocytes (top) and hemoglobin (bottom). Doses shown on each graph from left to right: 0.75 mg/kg, 1.5 mg/kg, 2.5 mg/kg, 3.75 mg/kg, and 5.0 mg/kg.

FIGS. 25A-25B are a series of graphs showing the expression of TGF-β receptors and ligands in megakaryocyte precursor cells from untreated mice. Murine bone marrow megakaryocyte precursors expressed activins, GDFs, BMPs and TGF-β ligands (FIG. 25B) and their cognate receptors (FIG. 25A). Receptors and ligands directly related to ActRIIA/B-mFc are in bold. ND, not detected.

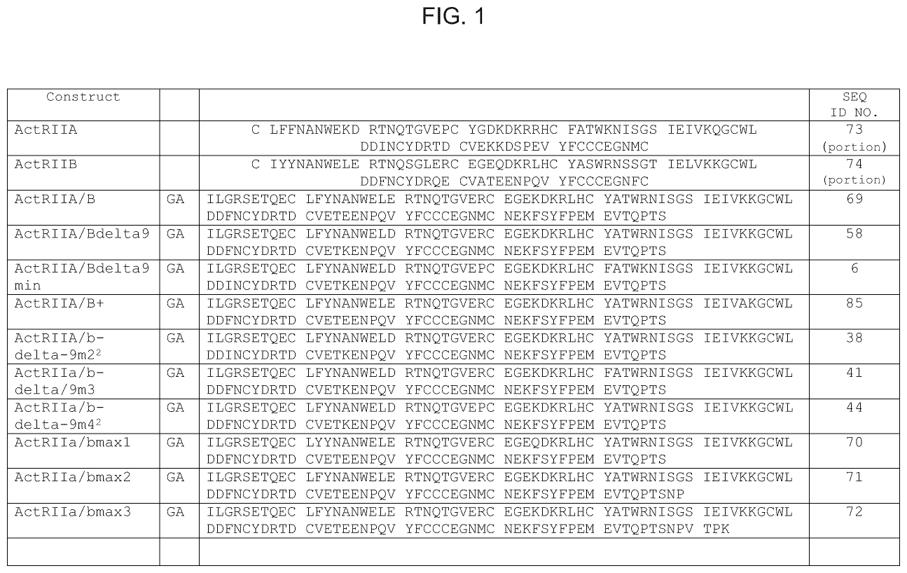

### DETAILED DESCRIPTION OF THE INVENTION

The invention features ActRII signaling inhibitors for use in methods of treating a cytopenia (e.g., anemia, thrombocytopenia, or neutropenia) associated with a myelodysplastic syndrome, CMML, or myelofibrosis and for use in treating myelofibrosis and reducing osteosclerosis, hepatosplenomegaly or splenomegaly, and bone marrow fibrosis in subjects with myelofibrosis. The ActRII signaling inhibitor can be an antibody that binds to an ActRII ligand, an anti-ActRII antibody, or an ActRII ligand trap, such as an ActRIIA ligand trap that includes an extracellular activin receptor type IIA (ActRIIA) variant. In some embodiments, an ActRIIA ligand trap includes an extracellular ActRIIA variant fused to a moiety (e.g., Fc domain monomer, an Fc domain, an albumin-binding peptide, a fibronectin domain, or a human serum albumin). An ActRIIA ligand trap including an extracellular ActRIIA variant fused to an Fc domain monomer may also form a dimer (e.g., homodimer or heterodimer) through the interaction between two Fc domain monomers. The extracellular ActRIIA variants described herein have weak binding affinity or no binding affinity to bone morphogenetic protein 9 (βMP9) compared to activins and myostatin. The ActRII signaling inhibitors described herein, such as an ActRIIA ligand including an extracellular ActRIIA variant described herein, can treat an MDS-associated or myelofibrosis-associated cytopenia by increasing red blood cell count, hemoglobin levels, hematocrit, reticulocytes, red blood cell production, proerythroblast numbers, erythroid progenitor maturation and/or differentiation (e.g., the maturation and/or differentiation of early-stage or late- (e.g., terminal) stage erythroid progenitors into proerythroblasts, reticulocytes, or red blood cells), late-stage precursor (erythroid precursor) maturation (e.g., terminal maturation, such as the maturation of reticulocytes into red blood cells or the maturation of erythroblasts into reticulocytes and/or red blood cells), by recruiting early-stage progenitors into the erythroid lineage, by reducing the accumulation of red blood cell progenitor cells (e.g., by stimulating progenitor cells to progress to maturation), by increasing the number of early-stage erythroid precursors and/or progenitors (e.g., by expanding the early-stage precursor and/or progenitor populations to provide a continuous supply of precursors to replenish polychromatic erythroblasts and allow for a continuous supply of maturing reticulocytes), by promoting the progression of erythroid precursors and/or progenitors through erythropoiesis, by increasing platelet levels (e.g., platelet count, megakaryocyte differentiation and/or maturation, megakaryocyte progenitor renewal, and/or platelet production), reducing the accumulation of platelet progenitor cells (e.g., by stimulating progenitor cells to progress to maturation), increasing neutrophil levels (e.g., neutrophil count, e.g., neutrophil production) or the differentiation and/or maturation of progenitor cells (e.g., myeloid progenitors, myeloblasts, or myelocytes) into neutrophils, and/or by reducing transfusion burden or promoting transfusion independence.

### ActRII Signaling

Activin type II receptors are single transmembrane domain receptors that modulate signals for ligands in the transforming growth factor β (TGF-β) superfamily. Ligands in the TGF-β superfamily are involved in a host of physiological processes, such as muscle growth, vascular growth, cell differentiation, homeostasis, and osteogenesis. Examples of ligands in the TGF-β superfamily include, e.g., activin A, activin B, inhibin, growth differentiation factors (GDFs) (e.g., GDF8, also known as myostatin, and GDF11), and bone morphogenetic proteins (βMPs) (e.g., BMP9).

TGF-β signaling pathways regulate hematopoiesis, with signaling pathways involving activins preventing the differentiation of red blood cell, platelet, and neutrophil progenitor cells in order to maintain progenitor cells in a quiescent state, and signaling pathways involving BMPs promoting differentiation of progenitor cells. Homeostasis of this process is essential to ensure that all cell types, including red cells, white cells, and platelets, are properly replenished in the blood. Relatedly, activin receptor ligand GDF11 has been found to be overexpressed in a mouse model of hemolytic anemia and associated with defects in red blood cell production. These data suggest that increased signaling through endogenous activin receptors, either due to increased expression of activin receptor ligands (e.g., activin A, activin B, myostatin) or increased expression of activin receptors themselves, could disrupt hematopoiesis. Methods that reduce or inhibit activin A, activin B, and/or myostatin signaling could, therefore, be used to promote hematopoiesis and treat diseases and conditions involving ineffective hematopoiesis, such as a cytopenia (e.g., anemia, thrombocytopenia, or neutropenia) associated with a myelodysplastic syndrome or myelofibrosis.

### ActRII Signaling Inhibitors

ActRII signaling inhibitors are agents that reduce or prevent the interaction of ActRII ligands with ActRIIA and/or ActRIIB, by either binding to the ligand or to the receptor. ActRII signaling inhibitors for use in the methods described herein provided herein below.

In some embodiments, the ActRII signaling inhibitor is an activin A antibody or an antigen binding fragment thereof. In some embodiments, the activin A antibody is garetosmab (also known as REGN-2477). Additional activin A antibodies that may be used in the methods described herein include those described in International Patent Application Publication Nos. WO2015017576, WO2013074557, and WO2008031061; US Patent Application No. US2015/0359850; and U.S. Pat. Nos. 9,718,881, 10,526,403, 8,309,082, 8,753,627, and 10,100,109, each of which is incorporated herein by reference.

In some embodiments, the activin A antibody or an antigen binding fragment thereof has a heavy chain variable region (HCVR) and a light chain variable region (LCVR) listed in Table 1 (e.g., an HCVR and an LCVR from the same row of Table 1). In some embodiments, the activin A antibody or antigen binding fragment thereof includes a HCVR sequence having at least 90% (e.g., at least 91%, 92%, 93%, 94%, 95%, 96%, 97%, 98%, 99%, or 100%) sequence identity to a HCVR sequence in Table 1, such as any one of SEQ ID NOs: 138, 140, 142, 143, 144, 146, 148, 150, 151, 172, and 174, and a LCVR sequence having at least 90% (e.g., at least 91%, 92%, 93%, 94%, 95%, 96%, 97%, 98%, 99%, or 100%) sequence identity to a LCVR sequence in Table 1, such as any one of SEQ ID NOs: 139, 141, 145, 147, 149, 173, and 175. In some embodiments, the activin A antibody or an antigen binding fragment thereof, apart from the light chain CDR1, CDR2, and CDR3 and the heavy chain CDR1, CDR2, and CDR3, has a HCVR and LCVR sequence having at least 90% sequence identity (e.g., 90%, 91%, 92%, 93%, 94%, 95%, 95%, 97%, 98%, 99% or more sequence identity) to a HCVR and LCVR sequence listed in Table 1. In some embodiments, the activin A antibody or an antigen binding fragment thereof has the light chain CDR1, CDR2, and CDR3 and the heavy chain CDR1, CDR2, and CDR3 sequences of an HCVR sequence and an LCVR sequence in Table 1. In some embodiments, the activin A antibody or antigen binding fragment thereof includes an HCVR sequence and an LCVR sequence from the same row of Table 1.

TABLE 1
Exemplary HCVR and LCVR sequences of activin A antibodies

| HCVR | LCVR |
|---|---|
| QVQLQESGPGLVKPSETLSLTCTVSGGSFSSHF | EIVLTQSPGTLSLSPGERATLSCRASQSVSSSYL |
| WSWIRQPPGKGLEWIGYILYTGGTSFNPSLKSR | AWYQQKPGQAPRLLIYGASSRATGIPDRFSGSG |
| VSMSVGTSKNQFSLKLSSVTAADTAVYYCARAR | SGTDFTLTISRLEPEDFAVYYCQQYGSSPWTFG |
| SGITFTGIIVPGSFDIWGQGTMVTVSS (SEQ ID | QGTKVEIK (SEQ ID NO: 139) |
| NO: 138) | |
| QVQLQQSGPGLLKPSQTLSLTCAISGDSVSSNS | DIVMTQSPDSLAVSLGERATINCKSSQSVLYSSN |
| AAWSWIRQSPSRGLEWLGRTYYRANWENDYA | NKNYLAWYQQKPGQPPTLLIYWASTRESGVPD |
| LSVKSRITINPVTSTNHFSLQLHSVTPEDTAVYY | RFSGSGSGTDFTLTISSLQAEDVAIYYCHQYFIT |
| CAREGALGYYFDSWGQGTLVTVSS (SEQ ID | PLTFGGGTKVEIK (SEQ ID NO: 141) |
| NO: 140) | |
| QVQLQESGPGLVKPSETLSITCTVSGGSFSSHF | EIVLTQSPGTLSLSPGERATLSCRASQSVSSSYL |
| WTWIRQPPGKGLEWIGYLHYSGGTSYNPSLKS | AWYQQKPGQAPRLLIYGASSRATGIPDRFSGSG |
| RVIISVDTSKNQFSLKLNSVTAADTAVYYCARAR | SGTDFTLTISRLEPEDFAVYYCQQYGSSPWTFG |
| SGITFGGLIVPGSFDIWGQGTMVTVSS | QGTKVEIK (SEQ ID NO: 139) |
| (SEQ ID NO: 142) | |
| QVQLQESGPGLVKPSETLSLTCTVSGGSFSSHF | EIVLTQSPGTLSLSPGERATLSCRASQSVSSSYL |
| WSWIRQPPGKGLEWIGYIYYSGGTHYNPSLESR | AWYQQKPGQAPRLLIYGASSRATGIPDRFSGSG |
| VTISVDTSKNQFSLKLNSVTAADTAVYYCARARS | SGTDFTLTISRLEPEDFAVYYCQQYGSSPWTFG |
| GITFGGLIVPGSFDIWGQGTMVTVSS | QGTKVEIK (SEQ ID NO: 139) |
| (SEQ ID NO: 143) | |
| EVQLVESGGGLVQPGRSLRLSCKASGFAFDDF | EIVLTQSPATLSLSPGERATLSCRASQTISTYLV |
| AMHWVRQAPGKGLEWVSGIVWNSGDIGYADS | WYRQRPGQAPSLLIYDASNRATDIPARFSGSGS |
| VKGRFTISRDNAKNSLYLQLNSLRTEDTALYFCV | GTDFTLTISSLEPEDFAVYYCQQRSNWPITFGQ |
| KDMVRGLMGFNYYGMDVWGQGTTVTVSS | GTRLEIK (SEQ ID NO: 145) |
| (SEQ ID NO: 144) | |
| QVQLVESGGGVVQPGRSLRLSCVASGFTVSSY | DIQMTQSPSSLSASVGDRVTITCRASQNINSFLS |
| GIHWVRQAPGKGLEWVSVIWYDGRNKDYADSV | WYQQKPGKAPKFLIYDASSIQSGAPSRFSGSGS |
| KGRFTISRDNSKNTVYLEMKGLRAEDTALYYCA | GTDFTLTISSLQPEDFATYYCQQSYSSPFTFGQ |
| RDKTGDFDSWGQGTLVTVSS (SEQ ID NO: 146) | GTKLEIK (SEQ ID NO: 147) |
| EVQLVESGGGLVQPGRSLTLSCAVSGFTFDDH | DIQMTQSPSSLSASVGDRVTITCRASQSISSYLN |
| AMHWVRQAPGKGLEWVSGISWNSVSIGYADSV | WYQQKPGKAPKLLIYAASSLQSGVPSRFSGSG |
| KGRFTISRDNAKTSLYLQMNSLRVDDTALYYCV | SGTDFTLTISSLQPEDFATYYCQQSYSTPPITFG |
| QDLNDILTGYPLFDFWGQGTLVTVSS (SEQ ID | QGTRLEIK (SEQ ID NO: 149) |
| NO: 148) | |
| QVQLVESGGGVVQPGRSLRLTCVASGFTVSSY | DIQMTQSPSSLSASVGDRVTITCRASQSISSYLN |
| GMHWVRQAPGKGLEWVAVMFYDESKKYYADS | WYQQKPGKAPKLLIYAASSLQSGVPSRFSGSG |
| VKGRFTISRDNSKNTLYLQMNSLRVEDTAVYYC | SGTDFTLTISSLQPEDFATYYCQQSYSTPPITFG |
| ARDEQLDFEYWGQGTLVTVSS (SEQ ID NO: | QGTRLEIK (SEQ ID NO: 149) |
| 150) | |
| EVQLVESGGGVVQPGGSLRLSCAASTFTFDDF | DIQMTQSPSSLSASVGDRVTITCRASQSISSYLN |
| AMHWVRQAPGKGLEWVSLITGDGGSTYYADPV | WYQQKPGKAPKLLIYAASSLQSGVPSRFSGSG |
| KGRFTISRDNSKNSLYLQMNSLRTEDTALYYCV | SGTDFTLTISSLQPEDFATYYCQQSYSTPPITFG |
| KDWWIAARPDYYYYGMDVWGQGTTVTVSS | QGTRLEIK (SEQ ID NO: 149) |
| (SEQ ID NO: 151) | |
| QVQLVQSGAEVKKPGASVKVSCKASGYTFTSY | SYEVTQAPSVSVSPGQTASITCSGDKLGDKYAC |
| GLSWVRQAPGQGLEWMGWIIPYNGNTNSAQKL | WYQQKPGQSPVLVIYQDSKRPSGIPERFSGSN |
| QGRVTMTTDTSTSTAYMELRSLRSDDTAVYFCA | SGNTATLTISGTQAMDEADYYCQAWDSSTAVF |
| RDRDYGVNYDAFDIWGQGTMVTVSS (SEQ ID | GGGTKLTVL (SEQ ID NO: 173) |
| NO: 172) | |
| EVQLVESGGGLVQPGGSLRLSCAASGFTFSSY | DIQMTQSPSSLSASVGDRVTITCRASQGIRNDL |
| WMSWVRQAPGKGLECVANIKQDGSEEYYVDS | GWYQQKPGKAPKRLIYAASSLQSGVPSRFSGS |
| VKGRFTISRDNAKNSLYLQMNSLRAEDTAVYYC | GSGTEFTLTISSLQPEDFATYYCRQQNTYPLTF |
| ARGSSSWYYYNYGMDVWGQGTTVTVSS (SEQ | GGGTKVEIK (SEQ ID NO: 175) |
| ID NO: 174) | |

In some embodiments, the activin A antibody or an antigen-binding fragment thereof, has the CDR sequences described in Table 2 (i.e., a light chain CDR1, CDR2, and CDR3 and a heavy chain CDR1, CDR2, and CDR3). In some embodiments, the activin A antibody or antigen binding fragment thereof includes a light chain variable CDR1 sequence having at least 90% (e.g., at least 91%, 92%, 93%, 94%, 95%, 96%, 97%, 98%, 99%, or 100%) sequence identity to a light chain variable CDR1 sequence in Table 2, such as any one of SEQ ID NOs: 155, 161, 179, and 185; a light chain variable CDR2 sequence having at least 90% (e.g., at least 91%, 92%, 93%, 94%, 95%, 96%, 97%, 98%, 99%, or 100%) sequence identity to a light chain variable CDR2 sequence in Table 2, such as SEQ ID NO: 180 or 186 or the amino acid sequence GAS or DAS; a light chain variable CDR3 sequence having at least 90% (e.g., at least 91%, 92%, 93%, 94%, 95%, 96%, 97%, 98%, 99%, or 100%) sequence identity to a light chain variable CDR3 sequence in Table 2, such as any one of SEQ ID NOs: 157, 163, 181, and 187; a heavy chain variable CDR1 sequence having at least 90% (e.g., at least 91%, 92%, 93%, 94%, 95%, 96%, 97%, 98%, 99%, or 100%) sequence identity to a heavy chain variable CDR1 sequence in Table 2, such as any one of SEQ ID NOs: 152, 158, 176, and 182; a heavy chain variable CDR2 sequence having at least 90% (e.g., at least 91%, 92%, 93%, 94%, 95%, 96%, 97%, 98%, 99%, or more) sequence identity to a heavy chain variable CDR2 sequence in Table 2, such as any one of SEQ ID NOs: 153, 159, 177, and 183; and a heavy chain variable CDR3 sequence having at least 90% (e.g., at least 91%, 92%, 93%, 94%, 95%, 96%, 97%, 98%, 99%, or more) sequence identity to a heavy chain variable CDR3 sequence in Table 2, such as any one of SEQ ID NOs: 154, 160, 178, and 184. In some embodiments, the activin A antibody or antigen binding fragment thereof includes a light chain CDR1, CDR2, and CDR3 sequence and a heavy chain CDR1, CDR2, and CDR3 sequence from the same row of Table 2.

TABLE 2
Exemplary CDR sequences of activin A antibodies

| HCDR1 | HCDR2 | HCDR3 | LCDR1 | LCDR2 | LCDR3 |
|---|---|---|---|---|---|
| GGSFSSHF | ILYTGGT | ARARSGITFT | QSVSSSY | GAS | QQYGSSPWT |
| (SEQ ID NO: | (SEQ ID NO: | GIIVPGSFDI | (SEQ ID NO: | | (SEQ ID NO: |
| 152) | 153) | (SEQ ID NO: | 155) | | 157) |
| | | 154) | | | |
| GFAFDDFA | IVWNSGDI | VKDMVRGLM | QTISTY (SEQ | DAS | QQRSNWPIT |
| (SEQ ID NO: | (SEQ ID NO: | GFNYYGMDV | ID NO: 161) | | (SEQ ID NO: |
| 158) | 159) | (SEQ ID NO: | | | 163) |
| | | 160) | | | |
| GYTFTSYGLS | WIIPYNGNTN | DRDYGVNYD | SGDKLGDKY | QDSKRPS | QAWDSSTAV |
| (SEQ ID NO: | SAQKLQG | AFDI (SEQ ID | AC (SEQ ID | (SEQ ID NO: | (SEQ ID NO: |
| 176) | (SEQ ID NO: | NO: 178) | NO: 179) | 180) | 181) |
| | 177) | | | | |
| GFTFSSYWM | NIKQDGSEEY | GSSSWYYYN | RASQGIRNDL | AASSLQS | RQQNTYPLT |
| S (SEQ ID | YVDSVKG | YGMDV (SEQ | G (SEQ ID | (SEQ ID NO: | (SEQ ID NO: |
| NO: 182) | (SEQ ID NO: | ID NO: 184) | NO: 185) | 186) | 187) |
| | 183) | | | | |

In some embodiments, the ActRII signaling inhibitor is a myostatin antibody or an antigen binding fragment thereof. In some embodiments, the myostatin antibody is Domagrozumab (also known as PF-06252616), Landogrozumab (also known as LY2495655), Trevogrumab (also known as REGN-1033), or SRK-015. Additional myostatin antibodies that may be used in the methods described herein include those described in International Patent Application Publication Nos. WO2007047112, WO2007044411, WO2006116269, WO2012024242, WO2016073853, WO2013186719, WO2009058346, WO2011150008, WO2016168613, WO2007024535, and WO2016098357, US Patent Application Nos. US20070178095 and US20210246198; and U.S. Pat. Nos. 10,000,560, 10,738,111, 7,632,499, 8,066,995, 7,635,760, 7,745,583, 7,745,583, 7,807,159, 8,999,343, 10,307,480, 8,992,913, 9,751,937, 9,409,981, 9,850,301, 8,840,894, 9,890,212, 9,260,515, 10,934,349, 8,871,209, 10,400,036, 7,888,486, and 8,372,625, each of which is incorporated herein by reference.

In some embodiments, the myostatin antibody or an antigen binding fragment thereof has a HCVR and a LCVR listed in Table 3 (e.g., an HCVR and an LCVR from the same row of Table 3). In some embodiments, the myostatin antibody or antigen binding fragment thereof includes a HCVR sequence having at least 90% (e.g., at least 91%, 92%, 93%, 94%, 95%, 96%, 97%, 98%, 99%, or 100%) sequence identity to a HCVR sequence in Table 3, such as any one of SEQ ID NOs: 164, 188, 201, 204-210, 222-228, 234, 250, 252, 254, 256, 258, 260, 262, 264, 266, 268, 270, 272, 298, 306, 308, 310, 312, 314, 316, 318, 320, 356, 371, 373, 387, 389, 391, 405, 407, 409, 411, 413, 415, 417, 419, 421-423, 425, 427, 429, 431, 433, 435, 437, 439, 441, 444, 446, and 448-476, and a LCVR sequence having at least 90% (e.g., at least 91%, 92%, 93%, 94%, 95%, 96%, 97%, 98%, 99%, or 100%) sequence identity to a LCVR sequence in Table 3, such as any one of SEQ ID NOs: 165, 189, 202, 203, 221, 229-233, 251, 253, 255, 257, 259, 261, 263, 265, 267, 269, 271, 273, 299, 307, 309, 311, 313, 315, 317, 319, 321, 358, 372, 374, 388, 390, 392, 406, 408, 410, 412, 414, 416, 418, 420, 424, 426, 428, 430, 432, 434, 436, 438, 440, 442, 443, 445, 447, and 477-486. In some embodiments, the myostatin antibody or an antigen binding fragment thereof, apart from the light chain CDR1, CDR2, and CDR3 and the heavy chain CDR1, CDR2, and CDR3, has a HCVR and LCVR sequence having at least 90% sequence identity (e.g., 90%, 91%, 92%, 93%, 94%, 95%, 95%, 97%, 98%, 99% or more sequence identity) to a HCVR and LCVR sequence listed in Table 3. In some embodiments, the myostatin antibody or an antigen binding fragment thereof has the light chain CDR1, CDR2, and CDR3 and the heavy chain CDR1, CDR2, and CDR3 sequences of an HCVR sequence and an LCVR sequence in Table 3. In some embodiments, the myostatin antibody or antigen binding fragment thereof includes an HCVR sequence and an LCVR sequence from the same row of Table 3. In some embodiments, the myostatin antibody or antigen binding fragment thereof includes an HCVR sequence of any one of SEQ ID NOs: 448-476 and an LCVR sequence of any one of SEQ ID NOs: 477-486.

TABLE 3
Exemplary HCVR and LCVR sequences of myostatin antibodies

| HCVR | LCVR |
|---|---|
| EVQVLESGGDLVQPGGSLRLSCAASGFTFSAY | DIQMTQSPASLSASVGDRVTITCRASQDISDYLA |
| AMTWVRQAPGKGLEWVSAISGSGGSAYYADSV | WYQQKPGKIPRLLIYTTSTLQSGVPSRFRGSGS |
| KGRFTISRDNSKNTVYLQMNSLRAEDTAVYYCA | GTDFTLTISSLQPEDVATYYCQKYDSAPLTFGG |
| KDGAWKMSGLDVWGQGTTVIVSS (SEQ ID NO: | GTKVEIK (SEQ ID NO: 165) |
| 164) | |
| EVQLVESGGGLVQPGGSLRLSCAASGLTFSRY | EIVLTQSPGTLSLSPGERATLSCRASSSVSSSYL |
| PMSWVRQAPGKGLVWVSAITSSGGSTYYSDTV | HWYQQKPGQAPRLLIYSTSNLVAGIPDRFSGSG |
| KGRFTISRDNAKNTLYLQMNSLRAEDTAVYYCA | SGTDFTLTISRLEPEDFAVYYCQHHSGYHFTFG |
| RLPDYWGQGTLVTVSS (SEQ ID NO: 188) | GGTKVEIK (SEQ ID NO: 189) |
| QVTLRESGPALVKPTQTLTLTCTFSGFSLRKVG | DIQMTQSPSSLSASVGDRVTITCSASSSISYMH |
| SSVSWIRQPPGKALEWLAHIYWDDDKRYNPSL | WYQQKPGKAPKLLIYDTSKLAVGVPSRFSGSGS |
| RNRLTISKDTSKNQVVLTMTNMDPVDTATYYCA | GTDFTLTISSLQPEDFATYYCQQWYRNPLTFGG |
| RRAITTVIGGGTFDYWGQGTTVTVSS (SEQ ID | GTKVEIK (SEQ ID NO: 202) |
| NO: 201) | |
| QVTLRESGPALVKPTQTLTLTCTFSGFSLRKVG | DIQMTQSPSSLSASVGDRVTITCSASSSISYMH |
| SSVSWIRQPPGKALEWLAHIYWDDDKRYNPSL | WYQQKPGKAPKLLIYDTSKLARGVPSRFSGSGS |
| RNRLTISKDTSKNQVVLTMTNMDPVDTATYYCA | GTDFTLTISSLQPEDFATYYCQQWYRNPLTFGG |
| RRAITTVIGGGTMDYWGQGTTVTVSS (SEQ ID | GTKVEIK (SEQ ID NO: 203) |
| NO: 204) | |
| QVTLRESGPALVKPTQTLTLTCTFSGFSLRKVG | DIQMTQSPSSLSASVGDRVTITCSASSSISYMH |
| SSVSWIRQPPGKALEWLAHIYWDDDKRYNPSL | WYQQKPGKAPKLLIYDTSKLARGVPSRFSGSGS |
| RNRLTISKDTSKNQVVLTMTNMDPVDTATYYCA | GTDFTLTISSLQPEDFATYYCQQWYRNPLTFGG |
| RRAITTVIGGGTFDYWGQGTTVTVSS (SEQ ID | GTKVEIK (SEQ ID NO: 203) |
| NO: 201) | |
| QVTLRESGPALVKPTQTLTLTCTFSGFSLRKVG | DIQMTQSPSSLSASVGDRVTITCSASSSISYMH |
| RSVSWIRQPPGKALEWLAHIYWDDDKRYNPSL | WYQQKPGKAPKLLIYDTSKLARGVPSRFSGSGS |
| RNRLTISKDTSKNQVVLTMTNMDPVDTATYYCA | GTDFTLTISSLQPEDFATYYCQQWYRNPLTFGG |
| RRAITTVIGGGTFDYWGQGTTVVSS (SEQ ID | GTKVEIK (SEQ ID NO: 203) |
| NO: 205) | |
| QVTLRESGPALVKPTQTLTLTCTFSGFSWRKVG | DIQMTQSPSSLSASVGDRVTITCSASSSISYMH |
| SSVSWIRQPPGKALEWLAHIYWDDDKRYNPSL | WYQQKPGKAPKLLIYDTSKLARGVPSRFSGSGS |
| RNRLTISKDTSKNQVVLTMTNMDPVDTATYYCA | GTDFTLTISSLQPEDFATYYCQQWYRNPLTFGG |
| RRAITTVIGGGTFDYWGQGTTVTVSS (SEQ ID | GTKVEIK (SEQ ID NO: 203) |
| NO: 206) | |
| QVTLRESGPALVKPTQTLTLTCTFSGFSLRKVG | DIQMTQSPSSLSASVGDRVTITCSASSSISYMH |
| SSVSWIRQPPGKALEWLAHIYWDDDLRYNPSLR | WYQQKPGKAPKLLIYDTSKLARGVPSRFSGSGS |
| NRLTISKDTSKNQVVLTMTNMDPVDTATYYCAR | GTDFTLTISSLQPEDFATYYCQQWYRNPLTFGG |
| RAITTVIGGGTFDYWGQGTTVTVSS (SEQ ID | GTKVEIK (SEQ ID NO: 203) |
| NO: 207) | |
| QVTLRESGPALVKPTQTLTLTCTFSGFSMRKVG | DIQMTQSPSSLSASVGDRVTITCSASSSISYMH |
| SSVSWIRQPPGKALEWLAHIYWDDDKRYNPSL | WYQQKPGKAPKLLIYDTSKLARGVPSRFSGSGS |
| RNRLTISKDTSKNQVVLTMTNMDPVDTATYYCA | GTDFTLTISSLQPEDFATYYCQQWYRNPLTFGG |
| RRAITTVIGGGTFDYWGQGTTVTVSS (SEQ ID | GTKVEIK (SEQ ID NO: 203) |
| NO: 208) | |
| QVTLRESGPALVKPTQTLTLTCTFSGFSLRKVG | DIQMTQSPSSLSASVGDRVTITCSASSSISYMH |
| SSISWIRQPPGKALEWLAHIYWDDDKRYNPSLR | WYQQKPGKAPKLLIYDTSKLARGVPSRFSGSGS |
| NRLTISKDTSKNQVVLTMTNMDPVDTATYYCAR | GTDFTLTISSLQPEDFATYYCQQWYRNPLTFGG |
| RAITTVIGGGTFDYWGQGTTVTVSS (SEQ ID | GTKVEIK (SEQ ID NO: 203) |
| NO: 209) | |
| QVTLRESGPALVKPTQTLTLTCTFSGFSLRKVG | DIQMTQSPSSLSASVGDRVTITCSASSSISYMH |
| SSVSWIRQPPGKALEWLAHIYWDDDKRYNPSL | WYQQKPGKAPKLLIYDTSKLARGVPSRFSGSGS |
| RNRLTISKDTSKNQVVLTMTNMDPVDTATYYCA | GTDFTLTISSLQPEDFATYYCQQWYRNPLTFGG |
| RRKITTVIGGGTFDYWGQGTTVTVSS (SEQ ID | GTKVEIK (SEQ ID NO: 203) |
| NO: 210) | |
| QLQLQESGPGLVKPSETLSLTCTVSGFSLRRVG | DIQMTQSPSSLSASVGDRVTITCSASSSISYMH |
| SSVSWIRQPPGKGLEWIGHIYWDDDKRYNPSL | WYQQKPGKAPKLLIYDTSKLARGVPSRFSGSGS |
| RNRVTISVDTSKNQFSLKLSSVTAADTAVYYCA | GTDFTLTISSLQPEDFATYYCQQWYSNPLTFGG |
| RRAITTVIGGGTFDYWGQGTLVTVSS (SEQ ID | GTKVEIK (SEQ ID NO: 221) |
| NO: 222) | |
| QLQLQESGPGLVKPSETLSLTCTVSGFSLRMVG | DIQMTQSPSSLSASVGDRVTITCSASSSISYMH |
| SSVSWIRQPPGKGLEWIGHIYWDDDKRLNPSLR | WYQQKPGKAPKLLIYDTSKLARGVPSRFSGSGS |
| NRVTISVDTSKNQFSLKLSSVTAADTAVYYCAR | GTDFTLTISSLQPEDFATYYCQQWYSNPLTFGG |
| RAITTVIGGGTFDYWGQGTLVTVSS (SEQ ID | GTKVEIK (SEQ ID NO: 221) |
| NO: 223) | |
| QLQLQESGPGLVKPSETLSLTCTVSGFSLRKVG | DIQMTQSPSSLSASVGDRVTITCSASSSISYMH |
| SSVSWIRQPPGKGLEWIGHIYWDDDLRYNPSLR | WYQQKPGKAPKLLIYDTSKLARGVPSRFSGSGS |
| NRVTISVDTSKNQFSLKLSSVTAADTAVYYCAR | GTDFTLTISSLQPEDFATYYCQQWYSNPLTFGG |
| RAITTVIGGGTFDYWGQGTLVTVSS (SEQ ID | GTKVEIK (SEQ ID NO: 221) |
| NO: 224) | |
| QLQLQESGPGLVKPSETLSLTCTVSGFSLRKVG | DIQMTQSPSSLSASVGDRVTITCSASSSISYMH |
| SSVSWIRQPPGKGLEWIGHIYWDDDKRYNPSL | WYQQKPGKAPKLLIYDTSKLARGVPSRFSGSGS |
| RNRVTISVDTSKNQFSLKLSSVTAADTAVYYCA | GTDFTLTISSLQPEDFATYYCQQWYSNPLTFGG |
| RRAITTVIGGGTFDMWGQGTLVTVSS (SEQ ID | GTKVEIK (SEQ ID NO: 221) |
| NO: 225) | |
| QLQLQESGPGLVKPSETLSLTCTVSGFSLRKVG | DIQMTQSPSSLSASVGDRVTITCSASSSISYMH |
| SSVSWIRQPPGKGLEWIGHIYWDDDKRYNPSL | WYQQKPGKAPKLLIYDTSKLARGVPSRFSGSGS |
| RNRVTISVDTSKNQFSLKLSSVTAADTAVYYCA | GTDFTLTISSLQPEDFATYYCQQWYSNPLTFGG |
| RRAITTVIGGGTFDLWGQGTLVTVSS (SEQ ID | GTKVEIK (SEQ ID NO: 221) |
| NO: 226) | |
| QLQLQESGPGLVKPSETLSLTCTVSGFSLRKVG | DIQMTQSPSSLSASVGDRVTITCSASSSISYMH |
| SSVSWIRQPPGKGLEWIGHIYWDDDKRLNPSLR | WYQQKPGKAPKLLIYDTSKLARGVPSRFSGSGS |
| NRVTISVDTSKNQFSLKLSSVTAADTAVYYCAR | GTDFTLTISSLQPEDFATYYCQQWYSNPLTFGG |
| RAITTVIGGGTFDYWGQGTLVTVSS (SEQ ID | GTKVEIK (SEQ ID NO: 221) |
| NO: 227) | |
| QLQLQESGPGLVKPSETLSLTCTVSGFSLRKVG | DIQMTQSPSSLSASVGDRVTITCSASSSISYSHW |
| SSVSWIRQPPGKGLEWIGHIYWDDDKRYNPSL | YQQKPGKAPKLLIYDTSKLARGVPSRFSGSGSG |
| RNRVTISVDTSKNQFSLKLSSVTAADTAVYYCA | TDFTLTISSLQPEDFATYYCQQWYSNPLTFGGG |
| RRAITTVIGGGTFDYWGQGTLVTVSS (SEQ ID | TKVEIK (SEQ ID NO: 229) |
| NO: 228) | |
| QLQLQESGPGLVKPSETLSLTCTVSGFSLRKVG | DIQMTQSPSSLSASVGDRVTITCSASSSISYAHW |
| SSVSWIRQPPGKGLEWIGHIYWDDDKRYNPSL | YQQKPGKAPKLLIYDTSKLARGVPSRFSGSGSG |
| RNRVTISVDTSKNQFSLKLSSVTAADTAVYYCA | TDFTLTISSLQPEDFATYYCQQWYSNPLTFGGG |
| RRAITTVIGGGTFDYWGQGTLVTVSS (SEQ ID | TKVEIK (SEQ ID NO: 230) |
| NO: 228) | |
| QLQLQESGPGLVKPSETLSLTCTVSGFSLRKVG | DIQMTQSPSSLSASVGDRVTITCSASSSISYMH |
| SSVSWIRQPPGKGLEWIGHIYWDDDKRYNPSL | WYQQKPGKAPKLLIYDTSKLARGVPSRFSGSGS |
| RNRVTISVDTSKNQFSLKLSSVTAADTAVYYCA | GTDFTLTISSLQPEDFATYYCQQWYLNPLTFGG |
| RRAITTVIGGGTFDYWGQGTLVTVSS (SEQ ID | GTKVEIK (SEQ ID NO: 231) |
| NO: 228) | |
| QLQLQESGPGLVKPSETLSLTCTVSGFSLRKVG | DIQMTQSPSSLSASVGDRVTITCSASSSISYMH |
| SSVSWIRQPPGKGLEWIGHIYWDDDKRYNPSL | WYQQKPGKAPKLLIYDTSKLARGVPSRFSGSGS |
| RNRVTISVDTSKNQFSLKLSSVTAADTAVYYCA | GTDFTLTISSLQPEDFATYYCQQWYENPLTFGG |
| RRAITTVIGGGTFDYWGQGTLVTVSS (SEQ ID | GTKVEIK (SEQ ID NO: 232) |
| NO: 228) | |
| QLQLQESGPGLVKPSETLSLTCTVSGFSLRKVG | DIQMTQSPSSLSASVGDRVTITCSASSSISYMH |
| SSVSWIRQPPGKGLEWIGHIYWDDDKRYNPSL | WYQQKPGKAPKLLIYDTSKLARGVPSRFSGSGS |
| RNRVTISVDTSKNQFSLKLSSVTAADTAVYYCA | GTDFTLTISSLQPEDFATYYCQQWYFNPLTFGG |
| RRAITTVIGGGTFDYWGQGTLVTVSS (SEQ ID | GTKVEIK (SEQ ID NO: 233) |
| NO: 228) | |
| QLQLQESGPGLVKPSETLSLTCTVSGFSLRKVG | DIQMTQSPSSLSASVGDRVTITCSASSSISYMH |
| SSVSWIRQPPGKGLEWIGHIYWDDDKRLNPSLR | WYQQKPGKAPKLLIYDTSKLARGVPSRFSGSGS |
| NRVTISVDTSKNQFSLKLSSVTAADTAVYYCAR | GTDFTLTISSLQPEDFATYYCQQWYLNPLTFGG |
| RAITTVIGGGTFDLWGQGTLVTVSS (SEQ ID | GTKVEIK (SEQ ID NO: 231) |
| NO: 234) | |
| EVQLVESGGGLVKPGGSLRLSCAASGFTFSSY | DIQMTQSPSSLSASVGDRVTITCRASQGIRNAL |
| SMNWVRQAPGKGLEWVSSISSSSSYIYYADSV | GWYQQKPGKAPQRLIYAASSLQSGVPSRFSGS |
| KGRFTISRDNAKNSLYLQMNSLRAEDTAVYYCA | GSGTEFTLTISSLQPEDFATYYCLQHNSYPRTF |
| RDRGYSYGLDYWGQGTLVTVSS (SEQ ID NO: | GQGTKVEIK (SEQ ID NO: 251) |
| 250) | |
| QVQLVQSGAEVKKPGASVKVSCKASGYTFTGY | DIVMTQSPLSLPVTPGEPASISCRSSQSLLHSNG |
| YMHWVRQAPGQGLEWMGWINPNNGGTNYAQ | YNYLDWYLQKSGQSPQLLIHLGSNRASGVPDR |
| KFQGRVTMTRDTSISTAYMELSRLRSDDTAVYH | FSGSGSGTDFTLKISRVEAEDVGVYYCMQVLQT |
| CARNTVGSGYYYYGMDVWGQGTTVT | PLTFGGGTKVEIK (SEQ ID NO: 253) |
| VSS (SEQ ID NO: 252) | |
| EVQLVESGGGLVKPGGSLRLSCAASGFTFSSY | DIQMTQSPSSLSASVGDRVTITCRASQGIRNAL |
| SMNWVRQAPGKGLEWVSSISSSSSYIYYADSV | GWYQQKPGKAPKRLIYAASSLQSGVPSRFSGS |
| KGRFTISRDNAKNSLYLQMNSLRAEDTAVYYCA | GSGTEFTLTISSLQPEDFATYYCLQHNSYPRTF |
| RDRGYSYGIDYWGQGTLVTVSS (SEQ ID NO: | GQGTKVEIK (SEQ ID NO: 255) |
| 254) | |
| EVQLVESGGGLVKPGGSLRLSCAASGFTFSSY | DIQMTQSPSSLSASVGDRVTLTCRASQGIRNAL |
| SMNWVRQAPGKGLEWVSSISSSSSYIYYADSV | GWYQQKPGKAPKRLIYGASSLQSGVPSRFSGS |
| KGRFTISRDNAKNSLYLQMNSLRAEDTAVYYCA | GSGTEFTLTISSLQPEDFATYYCLQHNSYPFTFG |
| RDRGYYYGMDVWGQGTTVTVSS (SEQ ID NO: | PGTKVDIK (SEQ ID NO: 257) |
| 256) | |
| EVQLVESGGGLVKPGGSLRLSCAASGFTFSSY | DMQMTQSPSSLSASVGDRVTITCRASQGIRNDL |
| SMNWVRQAPGKGLEWVSSISSSSSYIYYADSV | GWYQQKPGKAPKRLIYGASSLQSGVPSRFSGS |
| KGRFTISRDNAKNSLYLQMNSLRAEDTAVYYCA | GSGTEFTLTISSLQPEDSATYYCLQHNSYPLTFG |
| RDRGSYFLFDYWGQGTLVTVSS (SEQ ID NO: | GGTKVEIK (SEQ ID NO: 259) |
| 258) | |
| EVQLVESGGGLVKPGGSLRLSCAASGFTFSSY | DIQMTQSPSSLSASVGDRVTITCRASQAIRNDLG |
| SMNWVRQAPGKGLEWVSSISSSSSYIYYADSV | WYQQKPGKAPKRLIYAASSLQSGVPSRFSGSG |
| KGRFTISRDNAKNSLYLQMNSLRAEDTTVYYCA | SGTEFTLTISSLQPEDFATYYCLQHNSYPRTFG |
| RDRGYLYGMDVWGQGTTVTVSS (SEQ ID NO: | QGTKVEIK (SEQ ID NO: 261) |
| 260) | |
| QVQLVQSGAEVKKPGASVKVSCKASGYTFTGY | DIVMTQSPLSLPVTPGEPASISCRSSQSLLHSNG |
| YMHWVRQAPGQGLEWMGWINPNSGGTNYAQ | YNYLDWYLQKPGQSPQVLIYLVSNRASGVPDRF |
| KFQGRVTMTRDTSISTAYLELSRLRSDDTAVYY | SGSGSGTDFTLKISRVEAEDVGVYYCMQALQTP |
| CARNTVGTGYYYYGMDVWGQGTTVT | PTFGGGTKVEIK (SEQ ID NO: 263) |
| VSS (SEQ ID NO: 262) | |
| EVQLVESGGGLVKPGGSLRLSCAASGFTFSSY | DIQMTQSPSSLSASVGDRVTITCRASQGIRNDL |
| SMNWVRQAPGKGLEWVSSISSSSSYIYYADSV | GWYQQKPGKAPKRLIYAASSLQSGVPSRFSGS |
| KGRFTISRDNAKNSLYLQMNSLRAEDTAVYYCA | GSGTEFTLTISSLQPEDFATYYCLQLNSYPFTFG |
| RDRGYSYGLDYWGQGTLVTVSS (SEQ ID NO: | PGTKVDIK (SEQ ID NO: 265) |
| 264) | |
| EVQLLESGGGLIQPGGSLRLSCAASGFTFSSFA | EIVMTQSPATLSVSPGERATLSCRASQSVSSNL |
| MSWVRQAPGKGLEWVSTISGSGGYTFYADSVK | AWYQQKPGQAPRLLIYGASTRATGFPARFSGS |
| GRFTISRDNSKNTLYLQMNSLRAEDTAVYYCAK | GSGTEFTLTISSLQSEDFAIYYCQQYNNWPLTF |
| DGRYNWNYGAFDIWGQGTMVTVSS (SEQ ID | GGGTKVEIK (SEQ ID NO: 267) |
| NO: 266) | |
| EVQLLESGGGLIQPGGSLRLSCAASGFTFSSFA | EIVMTQSPATLSVSPGERATLSCRASQSVRSNL |
| MSWVRQAPGKGLEWVSTISGSGGSTYYADFVK | AWFQQKPGQAPRLLIYGASTRATGIPARFSGSG |
| GRFTISRDNSKNTLYLQMNSLRAEDTAVYYCAK | SGTEFTLTISSLQSEDFAVYYCQQYNNWPLTFG |
| DGRYNWNYGAFDIWGQGTMVTVSS (SEQ ID | GGTKVEIK (SEQ ID NO: 269) |
| NO: 268) | |
| EVQLLESGGGLVQPGGSLRLSCAASGFTFSSY | EIVMTQSPATLSVSPGERATLSCRTSQSVSSNL |
| AMSWVRQAPGKGLEWVSAISGSGGSTFYADSV | AWYQQKPGQAPRLLIYGASTRATGIPARFSGSG |
| TGRFTISRDNSKNTLFLQLNSLRAEDTAVYHCAK | SGTEFTLTISSLQSEDFAVYYCQQYNNWPLTFG |
| DGRFNWNYGASDIWGQGTMVTVSS (SEQ ID | GGTKVEIK (SEQ ID NO: 271) |
| NO: 270) | |
| EVQLLESGGGLVQPGGSLRLSCAASGFTFSSFA | EIVMTQSPATLSVSPGERATLSCRASQSVSSNL |
| MSWVRQAPGKGLEWVSTISGSGGYTFYADSVK | AWYQQKPGQAPRLLIYGASTRATGIPARFSGSG |
| GRFTISRDNSKNTLYLQMNSLRAEDTAVYYCAK | SGTEFTLTISSLQSEDFAVYYCQQYNNWPLTFG |
| DGRYNWNYGAFDIWGQGTMVTVSS (SEQ ID | GGTKVEIK (SEQ ID NO: 273) |
| NO: 272) | |
| EVQLVESGGGLVQPGGSLRLSCAASGFTFSRY | DIQMTQSPSSLSASVGDRVTITCKASQDINKYVA |
| WMNWVRQAPGKGLEWVAQIRLKSDNYATHYA | WYQQKPGKAPKLLIYYTSWLQPGVPSRFSGSG |
| ESVKGRFTISRDNAKNSLYLQMNSLRAEDTAVY | SGTDFTFTISSLQPEDIATYYCLQYDNLLYTFGQ |
| YCTEGLDYWGQGTTVTVSS (SEQ ID NO: 298) | GTKLEIK (SEQ ID NO: 299) |
| QIQLVQSGGGVVQPGRSLRLSCAASGFTFSSY | QPVLTQPPSASGTPGQRVTISCSGSSSNIGSNT |
| GMHWVRQAPGKGLEWVAVISYDGSNKYYADS | VHWYQQLPGTAPKLLIYSDNQRPSGVPDRFSG |
| VKGRFTISRDNSKNTLYLQMNSLRAEDTAVYYC | SKSGTSASLVISGLQSDDEADYYCAAWDDSLN |
| ARDLLVRFLEWSHYYGMDVWGQGTTVTVSS | GVFGGGTKLTVL (SEQ ID NO: 307) |
| (SEQ ID NO: 306) | |
| QVQLVESGGGVVQPGRSLRLSCAASGFTFSSY | QSVLTQPPSASGTPGQRVTISCSGSSSNIGSNT |
| GMHWVRQAPGKGLEWVAVISYDGSNKYYADS | VHWYQQLPGTAPKLLIYSDNQRPSGVPDRFSG |
| VKGRFTISRDNSKNTLYLQMNSLRAEDTAVYYC | SKSGTSASLAISGLQSEDEADYYCAAWDDSLNG |
| ARDLLVRFLEWSHYYGMDVWGQGTTVTVSS | VFGGGTKLTVL (SEQ ID NO: 309) |
| (SEQ ID NO: 308) | |
| QVQLQQSGGGLVQPGGSLRLSCAASGFTFSSY | DIQMTQSPSFLSASVGDRVTITCRASQGISSYLA |
| AMTWVRQAPGKGLEWVSAISGGGGATYYADS | WYQQKPGKAPKLLIYAASSLQSGVPSRFSGSG |
| VEGRFTISRDNSKNTLYLQMNSLRAEDTAIYYCA | SGTDFTLTISSLQPEDFATYYCQQSYSTPLTFGG |
| SPAGHICSGGSCQYYYYGMDVWGQGTTVTVS | GTRVDMARTVAAPSVF (SEQ ID NO: 311) |
| SGS (SEQ ID NO: 310) | |
| QVQLVQSGAEVKKPGSSVKVSCKASGGTFSSY | EIVMTQSPGTLSLSPGERATLSCRASQSVSSGY |
| AISWVRQAPGQGLEWMGGIIPIFGTANYAQKFQ | LAWYQQKAGQAPRLLIYGGSARAPGIPARFSGS |
| GRVTITADESTSTAYMELSSLRSEDTAVYYCAR | GSGTDFTLTISSLEPEDFAVYYCQQRSIWPLTFG |
| DRHLYRRGYSSVDGMDVWGQGTTVTVSSGS | GGTKVEIKRTVAAPSVF (SEQ ID NO: 313) |
| (SEQ ID NO: 312) | |
| QVQLVQSGAEVKKPGESLKISCKGSGYSFTSY | DIVMTQSPVSLPVTLGQPASISCRSSQSVVYRD |
| WIGWVRQMPGKGLEWMGIIYPGDSDTRYSPSF | GNTYLSWFLQRPGQSPRRLIYKVSNRDSGVPD |
| QGQVTISADKSISTAYLQWSSLKASDTAMYYCA | RFSGSGSATDFTLKISRVEAEDVGVYYCMQGSS |
| RHPIGSGYDYWGQGTLVTVSSGS (SEQ ID NO: | WPYTFGQGTKLEIKRTVAAPSVF (SEQ ID NO: |
| 314) | 315) |
| QVQLVQSGGGVVRPGGSLRLSCAASGFTFDDY | QSALTQPASVSGSPGQSITISCTGTSSDVGGYN |
| GMSWVRQAPGKGLEWVSGISWNSGSIGYADS | YISWYQQYPGKAPKLMIYDVSKRPSGVSNRFSG |
| VKGRFTISRDNAKNSLYLQMNSLRAEDTALYYC | SKSGNTASLTISGLQAEDEADYYCNSYTSSRTV |
| AKEISDTAMGFDYWGQGTLVTVSSGS (SEQ ID | VFGGGTKLTVLGQPKAAPSVTLFPPSS (SEQ ID |
| NO: 316) | NO: 317) |
| QVQLVQSGGGLVQPGGSLRLSCAASGFTFSNY | QSALTQPASVSGSPGQSITISCTGTSSDVGGYN |
| WMYWVRQAPGKGLVWVSNINSDGSSTSYADS | YVSWYQQHPGKAPKLLIYDVTKRPSGVSNRFS |
| VKGRFTISRDNAKNSLYLQMNSLRDEDTAVYYC | GSKSGNTASLTISGLQAEDEADYYCSSYTSSST |
| AREGYNAHYFDYWGQGTLVTVSSGS (SEQ ID | VVFGGGTKLTVLGQPKAAPSVTLFPPSSKASGA |
| NO: 318) | (SEQ ID NO: 319) |
| QIQLVQSGGGVVQPGRSLRLSCAASGFTFSSY | DIVMTQSPDSLAVSLGERATINCKSSQSVLYSSD |
| GMHWVRQAPGKGLEWVAVIWYDGSNKYYADS | NKNYLAWYQQKPGQPPKLLIYWASTRESGVPD |
| VKGRFTISRDNSKNTLYLQMNSLRAEDTAVYYC | RFSGSGSGTDFTLTISSLQAEDVAVYYCQQYYS |
| AKGLGYHPLFDYWGQGTLVTVSSGS (SEQ ID | TPSFGPGTKVDIKRTVAAPSVFKASGA (SEQ ID |
| NO: 320) | NO: 321) |
| EVQLLESGGGLVQPGGSLRLSCAASGFTFSSY | DIQMTQSPSSLSASVGDRVTITCKASQDVSTAV |
| AMSWVRQAPGKGLEWVSTISSGGSYTSYPDSV | AWYQQKPGKAPKLLIYSASYRYTGVPSRFSGS |
| KGRFTISRDNSKNTLYLQMNSLRAEDTAVYYCA | GSGTDFTLTISSLQPEDFATYYCQQHYSTPWTF |
| KQDYAMNYWGQGTLVTVSS (SEQ ID NO: 356) | GGGTKVEIK (SEQ ID NO: 357) |
| EVQLLESGGGLVQPGGSLRLSCAASGFTFSSY | DIQMTQSPSSLSASVGDRVTITCKASQDVSTAV |
| AMSWVRQAPGKGLEWVSTISSGGSYTSYPDSV | AWYQQKPGKAPKLLIYSASYRYTGVPSRFSGS |
| KGRFTISRDNSKNTLYLQMNSLRAEDTAVYYCA | GSGTDFTLTISSLQPEDFATYYCQQHYSTPWTF |
| KQDYAMNYWGQGTLVTVSS (SEQ ID NO: 356) | GQGTKVEIK (SEQ ID NO: 358) |
| QVQLVQSGAEVKKPGASVKVSCKASGYTFTSY | DIVMTQSPDSLAVSLGERATINCKSSQSLLNSAN |
| WMQWVRQAPGQGLEWMGAIYPGDGDTRYTQ | QKNYLAWYQQKPGQPPKLLIYFASTRESGVPD |
| KFKGRVTMTRDTSTSTVYMELSSLRSEDTAVYY | RFSGSGSGTDFTLTISSLQAEDVAVYYCQQHYN |
| CARMGGYDRYYFDYWGQGTLVTVSS (SEQ ID | TPLTFGGGTKVEIK (SEQ ID NO: 372) |
| NO: 371) | |
| QVQLQQSGAELARPGASVKLSCKASGYTFTSY | DIVMTQSPSSLAMSVGQKVTMSCKSSQSLLNSA |
| WMQWVKQRPGQGLEWIGAIYPGDGDTRYTQK | NQKNYLAWYQQKPGQSPKLLVYFASTRESGVP |
| FKGKATLTADKSSSTAYMQLSSLASEDSAVYYC | DRFIGSGSGTDFTLTISSVQAEDLADYFCQQHY |
| ARMGGYDRYYFDYWGQGTTLTVSS (SEQ ID | NTPLTFGAGTKLELK (SEQ ID NO: 374) |
| NO: 373) | |
| QVQLVESGGGVVQPGRSLRLSCAASGFTFSRY | DIVMTQAAPSIPVIPGESVSMSCRSSKSLLYSNG |
| GIHWVRQAPGKGLEWVAVISYDGSDEYYVDSV | HTYVYWFVQRPGQSPQLLIYRMSNLASGVPDR |
| KGRFSISRDNSKNTLYLQMNSLRPADSAVYYCV | FSGSGSGTAFTLRISRVEAEDVGVYYCMQNLEF |
| KGDLELGFDYWGQGTLVTVSS (SEQ ID NO: | PLTFGAGTKLELK (SEQ ID NO: 388) |
| 387) | |
| EVQVLESGGDLVQPGGSLRLSCAASGFTFSAY | DIQMTQSPASLSASVGDRVTITCRASQDISDYLA |
| AMTWVRQAPGKGLEWVSAISGSGGSAYYADSV | WYQQKPGKIPRLLIYTTSTLQSGVPSRFRGSGS |
| KGRFTISRDNSKNTVYLQMNSLRAEDTAVYYCA | GTDFTLTISSLQPEDVATYYCQKYDSAPLTFGG |
| KDGAWKMSGLDVWGQGTTVIVSS (SEQ ID NO: | GTKVEIK (SEQ ID NO: 390) |
| 389) | |
| QVQLVESGGGVVQPGKSLRLSCAASGFTFSSF | DIQMTQSPSSVSASVGDRVTITCRASQGISNWL |
| GMHWVRQAPGKGLEWVAVIGYDGGNEYYADS | AWYQQKPGKAPKLLIFAASSLQSGVPSRFSGSA |
| VKGRFTISRDNSKNTLNLQMSSLRAEDTAVYYC | SGTDFTLTINSLQPEDFATYYCQQANSFPLTFG |
| STISHYDILSGMDVWGRGTTVTVSS (SEQ ID | GGTKVEIKR (SEQ ID NO: 392) |
| NO: 391) | |
| QVQLVQSGGGLVQPGGSLRLSCAASGFTFNTY | DIQMTQSPATLSVSPGERATLSCRASQSVSSNL |
| AISWVRQAPGKGLEWVSTITGSGYNTYYADSVK | AWYHQKPGQAPRLLIYGVSTRATGIPARFSGNG |
| GRFTISRDNSKNTLYLQMSSLRAEDTAVFYCAK | SGTEFTLTISSLQSEDFAVYYCQQHNNWPLTFG |
| DSRYNWNYGIFDYWGQGTTVTVSS (SEQ ID | GGTKVEIKR (SEQ ID NO: 406) |
| NO: 405) | |
| QVQLVQSGPGLVKPSETLSLTCTVSGGSISNSN | DIQLTQSPSSLSASVGDRVTITCRASQDIRNDLG |
| YYWGWIRQPPGKGLEWIGTTYYSGTTYYNPSL | WYQQKPGKAPKRLIYAASSLQSGVPSRFSGSG |
| KSRVTISVDTSKNQFSLKLSSVTAADTAVYYCAR | SGTEFSLTLSSLQPEDFATYFCLQHHIYPWTFG |
| DYYDSSGYYYNWFDPWGQGTTVTVSS (SEQ | QGTKLEIKR (SEQ ID NO: 408) |
| ID NO: 407) | |
| QVQLVESGPGLVKPSETLSLTCTVYGGSISSGN | AIQMTQSPSSLSASVGDRVTITCRASQDIRHDLG |
| YYWGWIRQPPGKGLEWIGTIYYSGSAYYNPSLK | WYQQKPGKAPKRLIYAASSLQSGVPSRFSGSG |
| SRVTMSVDTSKNQFSLKLSSVTAADTAVYYCVR | SGTEFTLTISSLQPEDFATYYCLQHNTYPWTFG |
| DYYDSSGHYYNWFDPWGQGTTVTVSS (SEQ | QGTKVEIKR (SEQ ID NO: 410) |
| ID NO: 409) | |
| QVQLVQSGGGLVKPGGSLRLSCAASGFTFSSY | AIQMTQSPSSLSASVGDRVTITCRASQGIRNDL |
| SMNWVRQAPGKGLEWVSSISSSSSYIYYADSV | GWYQQKPGKAPKRLIYAASSLQSGVPSRFSGS |
| KGRFTISRDNAKNSLYLQMNSLRAEDTAVYYCA | GSGTEFTLTISSLQPEDFATYYCLQHNSYPYTFG |
| RDRGYTFGVDYWGQGTTVTVSS (SEQ ID NO: | QGTKVEIKR (SEQ ID NO: 412) |
| 411) | |
| QVQLVQSGPGLVKPSETLSLTCTVSGGSIITYSY | AIQMTQSPSSLSASVGDRVTITCRASQGIRNDL |
| YWGWIRQPPGKGLEWIGTIHHSGSTYYNPSLKS | GWYQQKPGKAPKRLIYAASSLQSGVPSRFSGS |
| RVTISVDTSKNQFSLTLSSVTAADTAVYYCARDY | GSGTEFTLTISSLQPEDFATYYCLQHNSSPWTF |
| YDSSGYYYNWFDPWGQGTMVTVSS (SEQ ID | GQGTKVEIKR (SEQ ID NO: 414) |
| NO: 413) | |
| QVHLKESGPTLVKPTQTLTLTCTFSGFSLSTSGV | AIQMTQSPSSLSASVGDRVTITCRASQGISNYLA |
| GVGWIRQPPGKALEWLALIYWNDDKRYSPSLK | WFQQKPGKAPKSLIYAASSLQSGVPSKFSGSG |
| SRLTITKDTSKNQVVLTMTNMDPVDTATYYCTH | SGTDFTLTISSLQPEDFATYYCQQYNSYPLTFG |
| TSRYNWHYGFLDYWGQGTTVTVSS (SEQ ID | GGTKVEIKR (SEQ ID NO: 416) |
| NO: 415) | |
| EVQLVQLGPGLVKPSETLSLTCTVSGGSISNSN | AIQMTQSPSSLSASVGDRVTITCRASQDIRNDLG |
| YYWGWIRQPPGKGLEWIGTTYYSGTTYYNPSL | WYQQKPGKAPKRLIYAASSLQSGVPSRFSGSG |
| KSRVTISVDTSKNQFSLKLSSVTAADTAVYYCAR | SGTEFSLTLSSLQPEDFATYFCLQHHIYPWTFG |
| DYYDSSGYYYNWFDPWGQGTTVTVSS (SEQ | QGTKVEIKR (SEQ ID NO: 418) |
| ID NO: 417) | |
| QVQLVEAGGGVVQPGRSLRLSCAASGFTFSRY | AIRLTQSPSSVSASVGDRVTITCRASQDISIWLA |
| GIHWVRQAPGKGLEWVAVISYDGTDEYYADSV | WYQQSPGKAPKLLINVASRLQSGVPSRFSGSG |
| KGRFTISRDNSKNTLYLQMNSLRPADSAVYYCA | SGTDFTLTINGLQPEDFVTYYCQQANSFPITFGQ |
| KGDLELGFDYWGQGTLVTVSS (SEQ ID NO: | GTRLATK (SEQ ID NO: 420) |
| 419) | |
| QVQLVQSGGGLVKPGGSLRLSCAASGFIFNTYT | DIQMTQSPSSLSASVGDRVTITCRASQDIRNDL |
| MNWVRQAPGKGLEWVSSITSRGTYIFYSDSLK | GWYQQKPGKAPKGLIYAASSLQSGVPSRFSGS |
| GRFTISRDNANNSLFLQMNSLRVEDTAVYYCSR | GSGTEFTLTISSLQPEDFATYHCLHYDFHPRTFG |
| DRGYTFGPDYWGQGTLVTVSS (SEQ ID NO: | QGTKVEIK (SEQ ID NO: 422) |
| 421) | |
| QVQLVESGGGVVQPGRSLRLSCAASGFTFSSY | EIVMTQSPATLSVSPGERATLSCRASQTFSSNL |
| AMHWVRQAPGKGLEWVAVIWYDGTNYYYADS | AWYQQKPGQAPRLLIYGASTRATGIPARFSGSG |
| VKGRFTISRDNSKNTLYLQMNSLRAEDTAVYYC | SGTEFTLTISSLQSEDFAVYYCQQYNKWPLTFG |
| ARDPLYYDILTGYSPDYYYGMDVWGQGTTVTV | GGTKVEIK (SEQ ID NO: 424) |
| SS (SEQ ID NO: 423) | |
| EVQLLESGGGLVQPGESLRLSCAASGFTFSSYA | EIVMTQSPATLSVSPGERATLSCRASQNVSSNL |
| MNWVRQAPGKGLEWVSTISGSGGYIYYADSVK | AWNKQKPGQAPRLLIYATSTRATGVPARFSASG |
| GRFTISRDNSKNTLYLQMNSLRAEDTAVYFCAK | SGTDFALTINSLQSEDFAVYYCQQYNNWPLTFG |
| DSRYNWNYGNFDYWGQGTLVTVSS (SEQ ID | GGTKVEIK (SEQ ID NO: 426) |
| NO: 425) | |
| EVQLLESGGGLVQPGGSLRLSCVASRFTFSSNA | EIVMTQSPATLSVSPGERATLSCRASQSVSSNL |
| MSWVRQAPGTGLEWVSAITGSGSRTYYADSVK | AWYQQKPGQAPRLLIYGASTRATGLPARFSGS |
| GRFTISRDNSKNTVYLQMNSLRGEDTAVYYCAK | GSGTDFTLTISSLQSEDFAVYYCQQYNNWPLTF |
| DQGGTWNYGDFDYWGQGTLVTVSS (SEQ ID | GGGTKVEIK (SEQ ID NO: 428) |
| NO: 427) | |
| QVQLVESGGDVVQPGRSLRLSCTASGFTFSSY | EIMMTQSPATLSVSPGERGTLSCRASQSVSSNL |
| GMHWVRQAPGRGLEWVAVISFDGKNKYYADS | AWYQQKPGQAPRLLIYGASTRASGIPARFSGSG |
| VKGRFTVSRDNSKNTLFLQMNSLRAEDTALYYC | SGTEFTLTVSSLQSEDFAVYYCQQYNNWPLTF |
| AKRIAATGYYYFYGLDVWGQGTTVTVSS (SEQ | GGGTKVEIK (SEQ ID NO: 430) |
| ID NO: 429) | |
| EVQLLESGGGLVQPGGSLRLSCAASGFTSITYA | EIVMTQSPATLSVSPGERATLSCRASQSVDSNL |
| MSWVRQAPGKGLEWVSAISVSGTNTYYADSVK | VWYQQKPGQVPRLLIYGASTRATGIPARFSGSG |
| GRFTISRDKSKNMLYLQMNSLRAEDTAVYYCAK | SGTEFTLTISSLQSEDFAVYYCQQYNKWPLTFG |
| DLLHNWKYGTFDIWGQGTMVTVSS | GGTKVEIK (SEQ ID NO: 432) |
| (SEQ ID NO: 431) | |
| EVQLLESGGGLVQPGGSLRLSCAASGFTSITYA | EIVMTQSPATLSVSPGERATLSCRASQSVSSNL |
| MSWVRQAPGKGLEWVSAISVSGTNTYYADSVK | AWNQQKPGQAPRLLIYGASTRATGIPARFSGSG |
| GRFTISRDKSKNMLYLQMNSLRAEDTAVYYCAK | SGTEFTLTISSLQSEDFAVYYCQQYNNWPMYTF |
| DLLHNWKYGTFDIWGQGTMVTVSS (SEQ ID | GQGTKLEIK (SEQ ID NO: 434) |
| NO: 433) | |
| QVQLVESGGGVVQPGRSLRLSCEASGFTFSSS | DIVNTQSPLSLPVTPGEPASISCRSSQSLLYGNG |
| GMHWVRQAPGKGLQWVAVISYDGNNKFYEDS | YNYLDWYLQKPGHSPQLLIYLGSNRGSGVPDR |
| VKGRLTISRDNSNNTLWLQMNSLRVEDTAVYYC | FSGSGSGTDFTLKISRVEAEDVGIYYCMQTLQT |
| AKSGGRVGAAFAYWGQGTLVTVSS (SEQ ID | PFTFGPGTKMYIK (SEQ ID NO: 436) |
| NO: 435) | |
| EVQLLESGGGLVQPGGSLRLSCAGSGITFSSYA | EIVMTQSPATLSMSPGERATLSCRASQSVSSNL |
| MSWVRQAPGKGLEWVSAISGNGGTTNYADSV | AWYQQKPGQAPRLLIYGASTRATGIPARFSGSG |
| KGRFTISRDNSKNTLYLQMNSLRAEDTAVYYCA | SGTEFTLTISSLQSEDFAVYYCQQYNNWPLTFG |
| KERILTSSWTRYGIMDVWGQGTTVTVSS (SEQ | GGTKLEIK (SEQ ID NO: 438) |
| ID NO: 437) | |
| QVQLQQWGAGLLKPSETLSLTCAVYGGSFSGY | DIQLTQSPSFLSASVGDRVTITCWASQGISSYLA |
| YWSWIRQPPGKGLEWIGEINHSGNTNYNPSLKS | WYQQKPGKAPKLLIYAASTLQSGVPSRFSGSGS |
| RVTISVDTSKNQFSLKLNSVTAADTAVYYCARR | GTEFTLTISSLQPEDFATYYCQQLNSYPLTFGG |
| EATVTPYFDYWGQGTLVTVSS (SEQ ID NO: | GTKVEIK (SEQ ID NO: 440) |
| 439) | |
| QVQLVESGGGVVQPGRSLRLSCVVSGFNFSRN | DIVMTQSPLSLPVTPGEPASISCRSSQSLLHSNG |
| GIHWVRQAPGKGLEWVAVISYDGRNKFYVESV | YNYLDWYLQKPGQSPQLMIYLGSHRASGVPDR |
| KGRFTISRDNSKNTLYLQMNSLRVEDTAVYYCA | FSGSGSGTDFTLKISRVEAEDVGVYYCIQVQQT |
| KSSIGGFFEYWGQGTLVTVSS (SEQ ID NO: | PITFGQGTRLEIK (SEQ ID NO: 442) |
| 441) | |
| QVQLVESGGGVVQPGRSLRLSCAASGFTFSRY | DIVMTQTPLSSPVTLGQPASISCRSSQSLVHSD |
| GIHWVRQAPGKGLEWVAVISYDGSDEYYVDSV | GNTYLSWLQQRPGQPPRLLIYKISNRFSGVPDR |
| KGRFSISRDNSKNTLYLQMNSLRPADSAVYYCV | FSGSGAGTDFTLKISRVEAEDVGVYFCMQATQF |
| KGDLELGFDYWGQGTLVTVSS (SEQ ID NO: | PYTFGQGTKLEIK (SEQ ID NO: 443) |
| 387) | |
| EVQLLESGGGLVQPGGSLRLSCAASGFTFSSY | DIQMTQSPSSLSASVGDRVTITCKASQDVSTAV |
| AMSWVRQAPGKGLEWVSTISSGGSYTSYPDSV | AWYQQKPGKAPKLLIYSASYRYTGVPSRFSGS |
| KGRFTISRDNSKNTLYLQMNSLRAEDTAVYYCA | GSGTDFTLTISSLQPEDFATYYCQQHYSTPWTF |
| KQDYAMNYWGQGTMVTVSS (SEQ ID NO: 444) | GQGTKVEIK (SEQ ID NO: 445) |
| EVQLVESGGGLVKPGGSLKLSCAASGFTFSSYA | DIEMTQSHKFMSTSVGDRVSITCKASQDVSTAV |
| MSWVRQTPEKRLEWVATISSGGSYTSYPDSVK | AWYQQKPGQSPKLLLYSASYRYTGVPDRFTGS |
| GRFTISRDNAKNTLYLQMSSLRSEDTAMYYCAR | GSGTDFTFTISSVQAEDLAVYYCQQHYSTPWTF |
| QDYAMNYWGQGTSVTVSS (SEQ ID NO: 446) | GGGTKLEIK (SEQ ID NO: 447) |
| EVQLLESGGGLVQPGGSLRLSCAASGFTFSSY | DIEMTQSHKFMSTSVGDRVSITCKASQDVSTAV |
| AMSWVRQAPGKGLEWVSTISSGGSYTSYPDSV | AWYQQKPGQSPKLLLYSASYRYTGVPDRFTGS |
| KGRFTISRDNSKNTLYLQMNSLRAEDTAVYYCA | GSGTDFTFTISSVQAEDLAVYYCQQHYSTPWTF |
| KQDYAMNYWGQGTMVTVSS (SEQ ID NO: 444) | GGGTKLEIK (SEQ ID NO: 447) |
| EVQLVESGGGLVKPGGSLKLSCAASGFTFSSYA | DIQMTQSPSSLSASVGDRVTITCKASQDVSTAV |
| MSWVRQTPEKRLEWVATISSGGSYTSYPDSVK | AWYQQKPGKAPKLLIYSASYRYTGVPSRFSGS |
| GRFTISRDNAKNTLYLQMSSLRSEDTAMYYCAR | GSGTDFTLTISSLQPEDFATYYCQQHYSTPWTF |
| QDYAMNYWGQGTSVTVSS (SEQ ID NO: 446) | GQGTKVEIK (SEQ ID NO: 445) |
| QVTLKESGPVLVKPTETLTLTCTVSGIDLSHDDIS | DIVMTQSPATLSLSPGERATLSCTSSQSVYHAN |
| WVRQAPGKGLEWVGIISYAGSMYYASWAKGRL | WLSWFQQKPGQPPKLLIYWASTLAYGVPSRFS |
| TISKDTSKNQVVLTMTNMDPVDTATYYCARGVP | GSGSGTDFTLTISSLQPEDAATYYCAGGYGGG |
| AYSHGGDKWGQGTLVTVSS (SEQ ID NO: 468) | RYAFGQGTKVEIK (SEQ ID NO: 483) |
| QVTLKESGPVLVKPTETLTLTCTVSGIDLSHTDIS | DIVMTQSPATLSLSPGERATLSCTSSQSVYHAN |
| WVRQAPGKGLEWVGIISYAGKTYYASWAKKRL | WLSWFQQKPGQPPKLLIYWASTLAYGVPSRFS |
| TISKDTSKNQVVLTMTNMDPVDTATYYCARGVP | GSGSGTDFTLTISSLQPEDAATYYCAGGYGGG |
| AYSHGGDLWGQGTLVTVSS (SEQ ID NO: 469) | RYAFGQGTKVEIK (SEQ ID NO: 483) |
| QVTLKESGPVLVKPTETLTLTCTVSGIDLSHTDIS | DIVMTQSPATLSLSPGERATLSCTSSQSVYHAN |
| WVRQAPGKGLEWVGIISYAGKKYYASWAKGRL | WLSWFQQKPGQPPKLLIYWASTLAYGVPSRFS |
| TISKDTSKNQVVLTMTNMDPVDTATYYCARGVP | GSGSGTDFTLTISSLQPEDAATYYCAGGYGGG |
| AYSHGGDLWGQGTLVTVSS (SEQ ID NO: 470) | RYAFGQGTKVEIK (SEQ ID NO: 483) |
| QVTLKESGPVLVKPTETLTLTCTVSGIDLSHTDIS | DIVMTQSPATLSLSPGERATLSCTSSQSVYHAN |
| WVRQAPGKGLEWVGIISYAGSKYKASWAKGRL | WLSWFQQKPGQPPKLLIYWASTLAYGVPSRFS |
| TISKDTSKNQVVLTMTNMDPVDTATYYCARGVP | GSGSGTDFTLTISSLQPEDAATYYCAGGYGGG |
| AYSHGGDLWGQGTLVTVSS (SEQ ID NO: 471) | RYAFGQGTKVEIK (SEQ ID NO: 483) |
| QVTLKESGPVLVKPTETLTLTCTVSGIDLSHDDIS | DIVMTQSPATLSLSPGERATLSCTSSQSVYHAN |
| WVRQAPGKGLEWVGIISYAGKKYYASWAKGRL | WLSWFQQKPGQPPKLLIYWASTLAYGVPSRFS |
| TISKDTSKNQVVLTMTNMDPVDTATYYCARGVP | GSGSGTDFTLTISSLQPEDAATYYCAGGYGGG |
| AYSHGGDLWGQGTLVTVSS (SEQ ID NO: 472) | RYAFGQGTKVEIK (SEQ ID NO: 483) |
| QVTLKESGPVLVKPTETLTLTCKVSGIDLSHDDI | DIVMTQSPATLSLSPGERATLSCTTSQSVYHEN |
| SWVRQAPGKGLEWVSIISYAGSTYYASWAKGR | WLSWFQQKPGQPPKLLIYWASTLAYGVPSRFS |
| LTISKDTSKNQVVLTMTNMDPVDTATYYCARGV | GSGSGTDFTLTISSLQPEDAATYYCAGGYGGG |
| PAYSHGGDLWGQGTLVTVSS (SEQ ID NO: 474) | RYAFGQGTKVEIK (SEQ ID NO: 484) |
| QVTLKESGPVLVKPTETLTLTCKVSGIDLSHEDIS | DIVMTQSPATLSLSPGERATLSCTTSQSVYHEN |
| WVRQAPGKGLEWVSIISYAGSTYYASWAKGRL | WLSWFQQKPGQPPKLLIYWASTLAYGVPSRFS |
| TISKDTSKNQVVLTMTNMDPVDTATYYCARGVP | GSGSGTDFTLTISSLQPEDAATYYCAGGYGGG |
| AYSHGGDLWGQGTLVTVSS (SEQ ID NO: 475) | RYAFGQGTKVEIK (SEQ ID NO: 484) |
| QVTLKESGPVLVKPTETLTLTCTVSGIDLSHDDIS | DIVMTQSPATLSLSPGERATLSCTSSQSVFHEN |
| WVRQAPGKGLEWVGIISYAGSTYYASWAKGRL | WLSWFQQKPGQPPKLLIYWASTLAWGVPSRFS |
| TISKDTSKNQVVLTMTNMDPVDTATYYCARGVP | GSGSGTDFTLTISSLQPEDAATYYCAGGYGGG |
| AYSHGGDLWGQGTLVTVSS (SEQ ID NO: 467) | RYAFGQGTKVEIK (SEQ ID NO: 485) |
| QVQLVESGGGLVQPGGSLRLSCAVSGIDLSHE | DIVMTQSPATLSLSPGERATLSCTTSQSVYHEN |
| DISWVRQAPGKGLEWVSIISYAGSKYYASWAKG | WLSWFQQKPGQPPKLLIYWASTLAYGVPSRFS |
| RLTISKDTSKNQVVLTMTNMDPVDTATYYCARG | GSGSGTDFTLTISSLQPEDAATYYCAGGYGGG |
| VPAYSHGGDLWGQGTLVTVSS | RYAFGQGTKVEIK (SEQ ID NO: 484) |
| (SEQ ID NO: 476) | |
| QVTLKESGPVLVKPTETLTLTCTVSGIDLSHDDIS | DIVMTQSPATLSLSPGERATLSCTSSQSVYHAN |
| WVRQAPGKGLEWVGIISYAGSTYYASWAKGRL | WLSWFQQKPGQPPKLLIYWASTLAYGVPSRFS |
| TISKDTSKNQVVLTMTNMDPVDTATYYCARGVP | GSGSGTDFTLTISSLQPEDAATYYCAGGYGGG |
| AYSHGGDLWGQGTLVTVSS (SEQ ID NO: 467) | RYAFGQGTKVEIK (SEQ ID NO: 483) |
| QVQLVESGGGLVQPGGSLRLSCAVSGIDLSHD | DIVMTQSPATLSLSPGERATLSCTTSQSVYHEN |
| DISWVRQAPGKGLEWVSIISYAGSTYYASWAKG | WLSWFQQKPGQPPKLLIYWASTLAYGVPSRFS |
| RLTISKDTSKNQVVLTMTNMDPVDTATYYCARG | GSGSGTDFTLTISSLQPEDAATYYCAGGYGGG |
| VPAYSHGGDLWGQGTLVTVSS | RYAFGQGTKVEIK (SEQ ID NO: 484) |
| (SEQ ID NO: 473) | |
| QVTLKESGPVLVKPTETLTLTCTVSGIDLSSYDIS | DIVMTQSPATLSLSPGERATLSCQSSQSVYDNN |
| WVRQAPGKGLEWVGIISYAGSTYYASWAKGRL | WLSWFQQKPGQPPKLLIYWASTLASGVPSRFS |
| TISKDTSKNQVVLTMTNMDPVDTATYYCARGVP | GSGSGTDFTLTISSLQPEDAATYYCAGGYGGGL |
| AYSTGGDLWGQGTLVTVSS (SEQ ID NO: 448) | YAFGQGTKVEIK (SEQ ID NO: 477) |
| QVTLKESGPVLVKPTETLTLTCTVSGIDLSHYDIS | DIVMTQSPATLSLSPGERATLSCQSSQSVYHNN |
| WVRQAPGKGLEWVGIISYAGSTYYASWAKGRL | WLSWFQQKPGQPPKLLIYWASTLASGVPSRFS |
| TISKDTSKNQVVLTMTNMDPVDTATYYCARGVP | GSGSGTDFTLTISSLQPEDAATYYCAGGYGGGL |
| AYSTGGDLWGQGTLVTVSS (SEQ ID NO: 449) | YAFGQGTKVEIK (SEQ ID NO: 478) |
| QVTLKESGPVLVKPTETLTLTCTVSGIDLSSYDIS | DIVMTQSPATLSLSPGERATLSCTSSQSVYHNN |
| WVRQAPGKGLEWVGIISHAGSTYYASWAKGRL | WLSWFQQKPGQPPKLLIYWASTLAYGVPSRFS |
| TISKDTSKNQVVLTMTNMDPVDTATYYCARGVP | GSGSGTDFTLTISSLQPEDAATYYCAGGYGGG |
| AYSTGGDLWGQGTLVTVSS (SEQ ID NO: 450) | RYAFGQGTKVEIK (SEQ ID NO: 479) |
| QVTLKESGPVLVKPTETLTLTCTVSGIDLSSYDIS | DIVMTQSPATLSLSPGERATLSCQSSQSVYHNN |
| WVRQAPGKGLEWVGIISYAGSTYYASWAKGRL | WLSWFQQKPGQPPKLLIYWASTLAFGVPSRFS |
| TISKDTSKNQVVLTMTNMDPVDTATYYCARGVP | GSGSGTDFTLTISSLQPEDAATYYCAGGYGGGL |
| AHSTGGDLWGQGTLVTVSS (SEQ ID NO: 451) | YAFGQGTKVEIK (SEQ ID NO: 480) |
| QVTLKESGPVLVKPTETLTLTCTVSGIDLSSYDIS | DIVMTQSPATLSLSPGERATLSCQTSQSVYHDN |
| WVRQAPGKGLEWVGIISYAGSTYYASWAKGRL | WLSWFQQKPGQPPKLLIYWASTLASGVPSRFS |
| TISKDTSKNQVVLTMTNMDPVDTATYYCARGVP | GSGSGTDFTLTISSLQPEDAATYYCAGGYGGG |
| AYSHGGDLWGQGTLVTVSS (SEQ ID NO: 452) | RYAFGQGTKVEIK (SEQ ID NO: 481) |
| QVTLKESGPVLVKPTETLTLTCTVSGIDLSHYDIS | DIVMTQSPATLSLSPGERATLSCTSSQSVYHDN |
| WVRQAPGKGLEWVGIISHAGSTYYASWAKGRL | WLSWFQQKPGQPPKLLIYWASTLAYGVPSRFS |
| TISKDTSKNQVVLTMTNMDPVDTATYYCARGVP | GSGSGTDFTLTISSLQPEDAATYYCAGGYGGGL |
| AYSTGGDLWGQGTLVTVSS (SEQ ID NO: 453) | YAFGQGTKVEIK (SEQ ID NO: 482) |
| QVTLKESGPVLVKPTETLTLTCTVSGIDLSSYDIS | DIVMTQSPATLSLSPGERATLSCTTSQSVYHEN |
| WVRQAPGKGLEWVGIISHAGSTYYASWAKGRL | WLSWYQQKPGQPPKLLIYWASTLAYGVPSRFS |
| TISKDTSKNQVVLTMTNMDPVDTATYYCARGVP | GSGSGTDFTLTISSLQPEDVATYYCAGGYGGG |
| AHSTGGDLWGQGTLVTVSS (SEQ ID NO: 454) | RYAFGQGTKVEIK (SEQ ID NO: 486) |
| QVTLKESGPVLVKPTETLTLTCTVSGIDLSHYDIS | |
| WVRQAPGKGLEWVGIISYAGSTYYASWAKGRL | |
| TISKDTSKNQVVLTMTNMDPVDTATYYCARGVP | |
| AHSTGGDLWGQGTLVTVSS (SEQ ID NO: 455) | |
| QVTLKESGPVLVKPTETLTLTCTVSGIDLSHYDIS | |
| WVRQAPGKGLEWVGIISHAGSTYYASWAKGRL | |
| TISKDTSKNQVVLTMTNMDPVDTATYYCARGVP | |
| AHSTGGDLWGQGTLVTVSS (SEQ ID NO: 456) | |
| QVTLKESGPVLVKPTETLTLTCTVSGIDLSHYDIS | |
| WVRQAPGKGLEWVGIISYAGSTYYASWAKGRL | |
| TISKDTSKNQVVLTMTNMDPVDTATYYCARGVP | |
| AYSHGGDLWGQGTLVTVSS (SEQ ID NO: 457) | |
| QVTLKESGPVLVKPTETLTLTCTVSGIDLSSYDIS | |
| WVRQAPGKGLEWVGIISHAGSTYYASWAKGRL | |
| TISKDTSKNQVVLTMTNMDPVDTATYYCARGVP | |
| AYSHGGDLWGQGTLVTVSS (SEQ ID NO: 458) | |
| QVTLKESGPVLVKPTETLTLTCTVSGIDLSSYDIS | |
| WVRQAPGKGLEWVGIISYAGSTYYASWAKGRL | |
| TISKDTSKNQVVLTMTNMDPVDTATYYCARGVP | |
| AHSHGGDLWGQGTLVTVSS (SEQ ID NO: 459) | |
| QVTLKESGPVLVKPTETLTLTCTVSGIDLSHYDIS | |
| WVRQAPGKGLEWVGIISHAGSTYYASWAKGRL | |
| TISKDTSKNQVVLTMTNMDPVDTATYYCARGVP | |
| AYSHGGDLWGQGTLVTVSS (SEQ ID NO: 460) | |
| QVTLKESGPVLVKPTETLTLTCTVSGIDLSSYDIS | |
| WVRQAPGKGLEWVGIISHAGSTYYASWAKGRL | |
| TISKDTSKNQVVLTMTNMDPVDTATYYCARGVP | |
| AHSHGGDLWGQGTLVTVSS (SEQ ID NO: 461) | |
| QVTLKESGPVLVKPTETLTLTCTVSGIDLSHYDIS | |
| WVRQAPGKGLEWVGIISYAGSTYYASWAKGRL | |
| TISKDTSKNQVVLTMTNMDPVDTATYYCARGVP | |
| AHSHGGDLWGQGTLVTVSS (SEQ ID NO: 462) | |
| QVTLKESGPVLVKPTETLTLTCTVSGIDLSHYDIS | |
| WVRQAPGKGLEWVGIISHAGSTYYASWAKGRL | |
| TISKDTSKNQVVLTMTNMDPVDTATYYCARGVP | |
| AHSHGGDLWGQGTLVTVSS (SEQ ID NO: 463) | |
| QVTLKESGPVLVKPTETLTLTCTVSGIDLSHTDIS | |
| WVRQAPGKGLEWVGIISYAGSTYYASWAKGRL | |
| TISKDTSKNQVVLTMTNMDPVDTATYYCARGVP | |
| AYSHGGDLWGQGTLVTVSS (SEQ ID NO: 464) | |
| QVTLKESGPVLVKPTETLTLTCTVSGIDLSHTDIS | |
| WVRQAPGKGLEWVGIISYAGSTYYMSWAKGRL | |
| TISKDTSKNQVVLTMTNMDPVDTATYYCARGVP | |
| AYSHGGDLWGQGTLVTVSS (SEQ ID NO: 465) | |
| QVTLKESGPVLVKPTETLTLTCTVSGIDLSHYDIS | |
| WVRQAPGKGLEWVGIISYAGSTYYASWAKGRL | |
| TISKDTSKNQVVLTMTNMDPVDTATYYCARGVP | |
| AYSHGGDLWGQGTLVTVSS (SEQ ID NO: 466) | |

In some embodiments, the myostatin antibody or an antigen-binding fragment thereof, has the CDR sequences described in Table 4, 5, or 6 (i.e., a light chain CDR1, CDR2, and CDR3 and a heavy chain CDR1, CDR2, and CDR3). In some embodiments, the myostatin antibody or antigen binding fragment thereof includes a light chain variable CDR1 sequence having at least 90% (e.g., at least 91%, 92%, 93%, 94%, 95%, 96%, 97%, 98%, 99%, or 100%) sequence identity to a light chain variable CDR1 sequence in Table 4 or Table 6, such as any one of SEQ ID NOs: 169, 193, 198, 238, 241, 303, 325, 330, 362, 378, 384, 396, 402, 826, 490, 493, and 343-346; a light chain variable CDR2 sequence having at least 90% (e.g., at least 91%, 92%, 93%, 94%, 95%, 96%, 97%, 98%, 99%, or 100%) sequence identity to a light chain variable CDR2 sequence in Table 4 or Table 6, such as any one of SEQ ID NOs: 194, 199, 239, 304, 326, 363, 379, 385, 827, and 491 or the amino acid sequence TTS, SDN, AAS, GGS, or DVT; a light chain variable CDR3 sequence having at least 90% (e.g., at least 91%, 92%, 93%, 94%, 95%, 96%, 97%, 98%, 99%, or 100%) sequence identity to a light chain variable CDR3 sequence in Table 4 or Table 6, such as any one of SEQ ID NOs: 171, 195, 200, 240, 245, 249, 305, 327, 364, 380, 386, 398, 404, 828, 492, and 350-355; a heavy chain variable CDR1 sequence having at least 90% (e.g., at least 91%, 92%, 93%, 94%, 95%, 96%, 97%, 98%, 99%, or 100%) sequence identity to a heavy chain variable CDR1 sequence in Table 4 or Table 5, such as any one of SEQ ID NOs: 166, 190 196, 235, 242, 246, 300, 322, 328, 359, 366, 375, 381, 393, 399, 487, and 332-334 or the amino acid sequence of X₁X₂DIS, in which X₁ is S or H, X₂ is Y, T, D or E; a heavy chain variable CDR2 sequence having at least 90% (e.g., at least 91%, 92%, 93%, 94%, 95%, 96%, 97%, 98%, 99%, or more) sequence identity to a heavy chain variable CDR2 sequence in Table 4 or Table 5, such as any one of SEQ ID NOs: 167, 191, 197, 236, 243, 247, 301, 323, 329, 360, 365, 376, 382, 394, 400, 824, 488, 335, and 336; and a heavy chain variable CDR3 sequence having at least 90% (e.g., at least 91%, 92%, 93%, 94%, 95%, 96%, 97%, 98%, 99%, or more) sequence identity to a heavy chain variable CDR3 sequence in Table 4 or Table 5, such as any one of SEQ ID NOs: 168, 192, 237, 244, 248, 302, 324, 361, 377, 383, 395, 401, 825, 489, and 337-342. In some embodiments, the myostatin antibody or antigen binding fragment thereof includes a light chain CDR1, CDR2, and CDR3 sequence and a heavy chain CDR1, CDR2, and CDR3 sequence from the same row of Table 4.

TABLE 4
Exemplary CDR sequences of myostatin antibodies

| HCDR1 | HCDR2 | HCDR3 | LCDR1 | LCDR2 | LCDR3 |
|---|---|---|---|---|---|
| GFTFSAYA | ISGSGGSA | AKDGAWKMS | QDISDY (SEQ | TTS | QKYDSAPLT |
| (SEQ ID NO: | (SEQ ID NO: | GLDV (SEQ | ID NO: 169) | | (SEQ ID NO: |
| 166) | 167) | ID NO: 168) | | | 171) |
| GLXFSRYXM | XXXXXGXST | LPDY (SEQ ID | RAXXSVSSS | STSNLXX, in | QXXXGYXXT, |
| S, in which X | XYSDTVKG, | NO: 192) | YLH, in which | which X at | in which X at |
| at position | in which X at | | X at position 3 | position 6 is A, | position 2 is Q, |
| 3 is | position 1 is A, | | is S or L and X | V, T, or M and | N, H, L, P or |
| T or N and X | R or H, X at | | at position 4 is | X at position 7 | W, X at |
| at position | position 2 is | | S or Q (SEQ | is S, A, T, N, | position 3 is Y |
| 8 is | or Q, X at | | ID NO: 193) | W, D or Y | or H, X at |
| G, T or P | position 3 is | | | (SEQ ID NO: | position 4 is S |
| (SEQ ID NO: | N, T or K, X at | | | 194) | or L, X at |
| 190) | position 4 is S | | | | position 7 is H, |
| | or A, X at | | | | F, Q or T, and |
| | position 5 is H, | | | | X at position 8 |
| | S, V, L, M, N, | | | | is F or W |
| | G, D, W, R, A, | | | | (SEQ ID NO: |
| | Y, K, P, T, I or | | | | 195) |
| | E, X at | | | | |
| | position 7 is G | | | | |
| | or S, and X at | | | | |
| | position 10 is | | | | |
| | Y or K (SEQ | | | | |
| | ID NO: 191) | | | | |
| GLTFSRYPM | AITSSGGSTY | LPDY (SEQ ID | RASSSVSSS | STSNLVA | QHHSGYHFT |
| S (SEQ ID | YSDTVKG | NO: 192) | YLH (SEQ ID | (SEQ ID NO: | (SEQ ID NO: |
| NO: 196) | (SEQ ID NO: | | NO: 198) | 199) | 200 |
| | 197) | | | | |
| GFSXRXVGX | HIYWDDDXR | RXITTVIGGG | SASSSISYMH | DTSKLAR | QQWYXNPLT, |
| SVS, in which | XNPSLRN, in | TXDX, in | (SEQ ID NO: | (SEQ ID NO: | in which X at |
| X at position 4 | which X at | which X at | 238) | 239) | position 5 is S, |
| is L, W, or M, | position 8 is K | position 2 is A | | | R, L, E, or F |
| X at position 6 | or L and X at | or K, X at | | | (SEQ ID NO: |
| is K, R, or M, | position 10 is | position 12 is | | | 240) |
| and X at | Y or L (SEQ ID | Mor F, and X | | | |
| position 9 is S | NO: 236) | at position 14 | | | |
| or R (SEQ ID | | is M or L (SEQ | | | |
| NO: 235) | | ID NO: 237) | | | |
| GFSXRXVGX | HIYWDDDXR | RXITTVIGGG | SASSSISYSH | DTSKLAR | QQWYXNPLT, |
| SVS, in which | XNPSLRN, in | TXDX, in | (SEQ ID NO: | (SEQ ID NO: | in which X at |
| X at position 4 | which X at | which X at | 241) | 239) | position 5 is S, |
| is L, W, or M, | position 8 is K | position 2 is A | | | R, L, E, or F |
| X at position 6 | or L and X at | or K, X at | | | (SEQ ID NO: |
| is K, R, or M, | position 10 is | position 12 is | | | 240 |
| and X at | Y or L (SEQ ID | Mor F, and X | | | |
| position 9 is S | NO: 236) | at position 14 | | | |
| or R (SEQ ID | | is M or L (SEQ | | | |
| NO: 235) | | ID NO: 237) | | | |
| GFSLRKVGR | HIYWDDDKR | RAITTVIGGG | SASSSISYMH | DTSKLAR | QQWYRNPLT |
| SVS (SEQ ID | YNPSLRN | TFDY (SEQ ID | (SEQ ID NO: | (SEQ ID NO: | (SEQ ID NO: |
| NO: 242) | (SEQ ID NO: | NO: 244) | 238) | 239) | 245) |
| | 243) | | | | |
| GFSLRKVGS | HIYWDDDKR | RAITTVIGGG | SASSSISYMH | DTSKLAR | QQWYLNPLT |
| SVS (SEQ ID | LNPSLRN | TFDL (SEQ ID | (SEQ ID NO: | (SEQ ID NO: | (SEQ ID NO: |
| NO: 246) | (SEQ ID NO: | NO: 248) | 238) | 239) | 249) |
| | 247) | | | | |
| RYWMN (SEQ | QIRLKSDNYA | GLDY (SEQ | KASQDINKYV | YTSWLQP | LQYDNLLYT |
| ID NO: 300) | THYAESVKG | ID NO: 302) | A (SEQ ID | (SEQ ID NO: | (SEQ ID NO: |
| | (SEQ ID NO: | | NO: 303) | 304) | 305) |
| | 301) | | | | |
| SSYGMH | VISYDGSNKY | DLLVRFLEWS | SGSSSNIGSN | SDNQRPS | AAWDDSLNG |
| (SEQ ID NO: | YADSVKG | HYYGMDV | TVH (SEQ ID | (SEQ ID NO: | V (SEQ ID |
| 322) | (SEQ ID NO: | (SEQ ID NO: | NO: 325) | 326) | NO: 327) |
| | 323) | 324) | | | |
| GFTFSSYGM | ISYDGSN | DLLVRFLEWS | SSNIGSNT | SDN | AAWDDSLNG |
| H (SEQ ID | (SEQ ID NO: | HYYGMDV | (SEQ ID NO: | | V (SEQ ID |
| NO: 328) | 329) | (SEQ ID NO: | 330) | | NO: 327) |
| | | 324) | | | |
| SYAMS (SEQ | TISSGGSYTS | QDYAMNY | KASQDVSTA | SASYRYT | QQHYSTPWT |
| ID NO: 359) | YPDSVKG | (SEQ ID NO: | VA (SEQ ID | (SEQ ID NO: | (SEQ ID NO: |
| | (SEQ ID NO: | 361) | NO: 362) | 363) | 364 |
| | 360) | | | | |
| SYAMS (SEQ | TISSGGSYTS | QDYAMNY | KASQDVSTA | SASYRYT | QQHYSTPWT |
| ID NO: 359) | (SEQ ID NO: | (SEQ ID NO: | VA (SEQ ID | (SEQ ID NO: | (SEQ ID NO: |
| | 365) | 361) | NO: 362) | 363) | 364) |
| GFTFSSYAM | TISSGGSYTS | QDYAMNY | KASQDVSTA | SASYRYT | QQHYSTPWT |
| S (SEQ ID | YPDSVKG | (SEQ ID NO: | VA (SEQ ID | (SEQ ID NO: | (SEQ ID NO: |
| NO: 366) | (SEQ ID NO: | 361) | NO: 362) | 363) | 364) |
| | 360) | | | | |
| GFTFSSYAM | TISSGGSYTS | QDYAMNY | KASQDVSTA | SASYRYT | QQHYSTPWT |
| S (SEQ ID | (SEQ ID NO: | (SEQ ID NO: | VA (SEQ ID | (SEQ ID NO: | (SEQ ID NO: |
| NO: 366) | 365) | 361) | NO: 362) | 363) | 364) |
| SYWMQ (SEQ | AIYPGDGDTR | ARMGGYDRY | KSSQSLLNSA | FASTRES | QQHYNTPLT |
| ID NO: 375) | YTQKFKG | YFDY (SEQ ID | NQKNYLA | (SEQ ID NO: | (SEQ ID NO: |
| | (SEQ ID NO: | NO: 377) | (SEQ ID NO: | 379) | 380) |
| | 376) | | 378) | | |
| GYTFTSYWM | AIYPGDGDT | ARMGGYDRY | KSSQSLLNSA | FASTRES | QQHYNTPLT |
| Q (SEQ ID | (SEQ ID NO: | YFDY (SEQ ID | NQKNYLA | (SEQ ID NO: | (SEQ ID NO: |
| NO: 381) | 382) | NO: 383) | (SEQ ID NO: | 385) | 386) |
| | | | 384) | | |
| GFTFSAYA | ISGSGGSA | AKDGAWKMS | QDISDY (SEQ | TTS | QKYDSAPLT |
| (SEQ ID NO: | (SEQ ID NO: | GLDV (SEQ | ID NO: 396) | | (SEQ ID NO: |
| 393) | 394) | ID NO: 395) | | | 398) |
| GFTFSSFG | IGYDGGNE | STISHYDILSG | QGISNW | AAS | QQANSFPLT |
| (SEQ ID NO: | (SEQ ID NO: | MDV (SEQ ID | (SEQ ID NO: | | (SEQ ID NO: |
| 399) | 400) | NO: 401) | 402) | | 404) |
| X1X2DIS, in | IISX1AGX2X3Y | GVPAX1SX2G | X1X2SQX3VX4 | WAX1TLAX2 | AGGYGGGX1 |
| which X1 is S | X4X5X6W | GDX3 (SEQ ID | X5X6NWLS | (SEQ ID NO: | YA (SEQ ID |
| or H, X2 is Y, | AKX7 (SEQ ID | NO: 825), in | (SEQ ID NO: | 827), in which | NO: 828), in |
| T, D or E | NO: 824), in | which X1 is Y | 826), in which | X1 is S or E, | which X1 is L |
| | which X1 is Y | or H, X2 is T or | X1 is Q or T, | X2 is S, Y, | or R |
| | or H, X2 is S or | H, X3 is L or K | X2 is S or T, | F or W | |
| | K, X3 is T, M or | | X3 is S or E, | | |
| | K, X4 is Y or K, | | X4 is Y or F, | | |
| | X5 is A, M or E, | | X5 is D or H, | | |
| | X6 is S or E, | | X6 is N, D, A | | |
| | X7 is G or K | | or E | | |
| HDDIS (SEQ | IISYAGSTYYA | GVPAYSHGG | TSSQSVYHA | WASTLAY | AGGYGGGRY |
| ID NO: 487) | SWAKG (SEQ | DL (SEQ ID | NWLS (SEQ | (SEQ ID NO: | A (SEQ ID |
| | ID NO: 488) | NO: 489) | ID NO: 490) | 491) | NO: 492) |
| HDDIS (SEQ | IISYAGSTYYA | GVPAYSHGG | TTSQSVYHE | WASTLAY | AGGYGGGRY |
| ID NO: 487) | SWAKG (SEQ | DL (SEQ ID | NWLS (SEQ | (SEQ ID NO: | A (SEQ ID |
| | ID NO: 488) | NO: 489) | ID NO: 493) | 491) | NO: 492) |

TABLE 5
Exemplary heavy chain CDR sequences
of myostatin antibodies

| HCDR1 | HCDR2 | HCDR3 |
|---|---|---|
| SSYGMH | VISYDGSNK | DLLVRFLEW |
| (SEQ ID NO: 322) | YYADSVKG | SHYYGMDV |
| | (SEQ ID NO: 323) | (SEQ ID NO: 324) |
| GFTFSSYGMH | ISYDGSN | ASPAGHICSGGSC |
| (SEQ ID NO: 328) | (SEQ ID NO: 329) | QYYYYGMDV |
| | | (SEQ ID NO: 337) |
| GFTFSSYA | ISGGGGAT | ARDRHLYRRGYSS |
| (SEQ ID NO: 332) | (SEQ ID NO: 335) | VDGMDV |
| | | (SEQ ID NO: 338) |
| GFTFDDYG | INSDGSST | ARHPIGSGYDY |
| (SEQ ID NO: 333) | (SEQ ID NO: 336) | (SEQ ID NO: 339) |
| GFTFSNYW | | AKEISDTAMGFDY |
| (SEQ ID NO: 334) | | (SEQ ID NO: 340) |
| | | AREGYNAHYFDY |
| | | (SEQ ID NO: 341) |
| | | AKGLGYHPLFDY |
| | | (SEQ ID NO: 342) |

TABLE 6
Exemplary light chain CDR sequences
of myostatin antibodies

| LCDR1 | LCDR2 | LCDR3 |
|---|---|---|
| SGSSSNIGSNTVH | SDNQRPS | AAWDDSLNGV |
| (SEQ ID | (SEQ ID | (SEQ ID |
| NO: 325) | NO: 326) | NO: 327) |
| SSNIGSNT | SDN | QQSYSTPLT |
| (SEQ ID | | (SEQ ID |
| NO: 330) | | NO: 350) |
| QGISSY | AAS | QQRSIWPLT |
| (SEQ ID | | (SEQ ID |
| NO: 343) | | NO: 351) |
| QSVSSGY | GGS | MQGSSWPYT |
| (SEQ ID | | (SEQ ID |
| NO: 344) | | NO: 352) |
| QSVVYRDGNTY | DVT | NSYTSSRTVV |
| (SEQ ID | | (SEQ ID |
| NO: 345) | | NO: 353) |
| QSVLYSSDNKNY | | SSYTSSSTVV |
| (SEQ ID | | (SEQ ID |
| NO: 346) | | NO: 354) |
| | | QQYYSTPS |
| | | (SEQ ID |
| | | NO: 355) |

In some embodiments, the myostatin antibody or an antigen-binding fragment thereof, has a heavy chain and light chain sequence having at least 90% sequence identity (e.g., 90%, 91%, 92%, 93%, 94%, 95%, 95%, 97%, 98%, 99% or 100% sequence identity) to a heavy chain and light chain sequence provided in Table 7. In some embodiments, the myostatin antibody or an antigen binding fragment thereof, has a heavy chain and light chain sequence from the same row of Table 7. In some embodiments, the heavy chain and light chain have the sequence of SEQ ID NOs: 274 and 275; 276 and 277; 278 and 279; 280 and 281; 282 and 283; 284 and 285; 286 and 287; 288 and 289; 290 and 291; 292 and 293; 294 and 295; 296 and 297; 367 and 368; or 369 and 370 (e.g., the heavy chain has at least 90% sequence identity (e.g., 90%, 91%, 92%, 93%, 94%, 95%, 95%, 97%, 98%, 99% or 100% sequence identity) to the sequence of the first SEQ ID NO: in each pair and the light chain has at least 90% sequence identity (e.g., 90%, 91%, 92%, 93%, 94%, 95%, 95%, 97%, 98%, 99% or 100% sequence identity) to the sequence of the second SEQ ID NO: in each pair).

TABLE 7
Exemplary heavy and light chain sequences of myostatin antibodies

| Heavy chain | Light chain |
|---|---|
| EVQLVESGGGLVKPGGSLRLSCAASGFTFSSY | DIQMTQSPSSLSASVGDRVTITCRASQGIRNAL |
| SMNWVRQAPGKGLEWVSSISSSSSYIYYADSV | GWYQQKPGKAPQRLIYAASSLQSGVPSRFSGS |
| KGRFTISRDNAKNSLYLQMNSLRAEDTAVYYCA | GSGTEFTLTISSLQPEDFATYYCLQHNSYPRTF |
| RDRGYSYGLDYWGQGTLVTVSSASTKGPSVFP | GQGTKVEIKRTVAAPSVFIFPPSDEQLKSGTASV |
| LAPCSRSTSESTAALGCLVKDYFPEPVTVSWNS | VCLLNNFYPREAKVQWKVDNALQSGNSQESVT |
| GALTSGVHTFPAVLQSSGLYSLSSVVTVPSSNF | EQDSKDSTYSLSSTLTLSKADYEKHKVYACEVT |
| GTQTYTCNVDHKPSNTKVDKTVERKCCVECPPC | HQGLSSPVTKSFNRGEC (SEQ ID NO: 275) |
| PAPPVAGPSVFLFPPKPKDTLMISRTPEVTCVV | |
| VDVSHEDPEVQFNWYVDGVEVHNAKTKPREEQ | |
| FNSTFRVVSVLTVVHQDWLNGKEYKCKVSNKG | |
| LPAPIEKTISKTKGQPREPQVYTLPPSREEMT | |
| KNQVSLTCLVKGFYPSDIAVEWESNGQPENNY | |
| KTTPPMLDSDGSFFLYSKLTVDKSRWQQGNVF | |
| SCSVMHEALHNHYTQKSLSLSPGK (SEQ ID NO: | |
| 274) | |
| QVQLVQSGAEVKKPGASVKVSCKASGYTFTGY | DIVMTQSPLSLPVTPGEPASISCRSSQSLLHSNG |
| YMHWVRQAPGQGLEWMGWINPNNGGTNYAQK | YNYLDWYLQKSGQSPQLLIHLGSNRASGVPDR |
| FQGRVTMTRDTSISTAYMELSRLRSDDTAVYH | FSGSGSGTDFTLKISRVEAEDVGVYYCMQVLQT |
| CARNTVGSGYYYYGMDVWGQGTTVTVSSASTK | PLTFGGGTKVEIKRTVAAPSVFIFPPSDEQLKSG |
| GPSVFPLAPCSRSTSESTAALGCLVKDYFPEP | TASVVCLLNNFYPREAKVQWKVDNALQSGNSQ |
| VTVSWNSGALTSGVHTFPAVLQSSGLYSLSSV | ESVTEQDSKDSTYSLSSTLTLSKADYEKHKVYA |
| VTVPSSNFGTQTYTCNVDHKPSNTKVDKTVER | CEVTHQGLSSPVTKSFNRGEC (SEQ ID NO: |
| KCCVECPPCPAPPVAGPSVFLFPPKPKDTLMI | 277) |
| SRTPEVTCVVVDVSHEDPEVQFNWYVDGVEVH | |
| NAKTKPREEQFNSTFRVVSVLTVVHQDWLNGK | |
| EYKCKVSNKGLPAPIEKTISKTKGQPREPQVY | |
| TLPPSREEMTKNQVSLTCLVKGFYPSDIAVEW | |
| ESNGQPENNYKTTPPMLDSDGSFFLYSKLTVD | |
| KSRWQQGNVFSCSVMHEALHNHYTQKSLSLSP | |
| GK | |
| (SEQ ID NO: 276) | |
| EVQLVESGGGLVKPGGSLRLSCAASGFTFSSY | DIQMTQSPSSLSASVGDRVTITCRASQGIRNAL |
| SMNWVRQAPGKGLEWVSSISSSSSYIYYADSV | GWYQQKPGKAPKRLIYAASSLQSGVPSRFSGS |
| KGRFTISRDNAKNSLYLQMNSLRAEDTAVYYCA | GSGTEFTLTISSLQPEDFATYYCLQHNSYPRTF |
| RDRGYSYGIDYWGQGTLVTVSSASTKGPSVFP | GQGTKVEIKRTVAAPSVFIFPPSDEQLKSGTASV |
| LAPCSRSTSESTAALGCLVKDYFPEPVTVSWNS | VCLLNNFYPREAKVQWKVDNALQSGNSQESVT |
| GALTSGVHTFPAVLQSSGLYSLSSVVTVPSSNF | EQDSKDSTYSLSSTLTLSKADYEKHKVYACEVT |
| GTQTYTCNVDHKPSNTKVDKTVERKCCVECPPC | HQGLSSPVTKSFNRGEC (SEQ ID NO: 279) |
| PAPPVAGPSVFLFPPKPKDTLMISRTPEVTCVV | |
| VDVSHEDPEVQFNWYVDGVEVHNAKTKPREEQ | |
| FNSTFRVVSVLTVVHQDWLNGKEYKCKVSNKG | |
| LPAPIEKTISKTKGQPREPQVYTLPPSREEMT | |
| KNQVSLTCLVKGFYPSDIAVEWESNGQPENNY | |
| KTTPPMLDSDGSFFLYSKLTVDKSRWQQGNVF | |
| SCSVMHEALHNHYTQKSLSLSPGK | |
| (SEQ ID NO: 278) | |
| EVQLVESGGGLVKPGGSLRLSCAASGFTFSSY | DIQMTQSPSSLSASVGDRVTLTCRASQGIRNAL |
| SMNWVRQAPGKGLEWVSSISSSSSYIYYADSV | GWYQQKPGKAPKRLIYGASSLQSGVPSRFSGS |
| KGRFTISRDNAKNSLYLQMNSLRAEDTAVYYCA | GSGTEFTLTISSLQPEDFATYYCLQHNSYPFTFG |
| RDRGYYYGMDVWGQGTTVTVSSASTKGPSVF | PGTKVDIKRTVAAPSVFIFPPSDEQLKSGTASVV |
| PLAPCSRSTSESTAALGCLVKDYFPEPVTVSWN | CLLNNFYPREAKVQWKVDNALQSGNSQESVTE |
| SGALTSGVHTFPAVLQSSGLYSLSSVVTVPSSN | QDSKDSTYSLSSTLTLSKADYEKHKVYACEVTH |
| FGTQTYTCNVDHKPSNTKVDKTVERKCCVECP | QGLSSPVTKSFNRGEC (SEQ ID NO: 281) |
| PCPAPPVAGPSVFLFPPKPKDTLMISRTPEVTCV | |
| VVDVSHEDPEVQFNWYVDGVEVHNAKTKPREE | |
| QFNSTFRVVSVLTVVHQDWLNGKEYKCKVSNK | |
| GLPAPIEKTISKTKGQPREPQVYTLPPSREEMTK | |
| NQVSLTCLVKGFYPSDIAVEWESNGQPENNYKT | |
| TPPMLDSDGSFFLYSKLTVDKSRWQQGNVFSC | |
| SVMHEALHNHYTQKSLSLSPGK (SEQ ID NO: | |
| 280) | |
| EVQLVESGGGLVKPGGSLRLSCAASGFTFSSY | DIQMTQSPSSLSASVGDRVTITCRASQAIRNDLG |
| SMNWVRQAPGKGLEWVSSISSSSSYIYYADSV | WYQQKPGKAPKRLIYAASSLQSGVPSRFSGSG |
| KGRFTISRDNAKNSLYLQMNSLRAEDTTVYYCA | SGTEFTLTISSLQPEDFATYYCLQHNSYPRTFG |
| RDRGYLYGMDVWGQGTTVTVSSASTKGPSVFP | QGTKVEIKRTVAAPSVFIFPPSDEQLKSGTASVV |
| LAPCSRSTSESTAALGCLVKDYFPEPVTVSWNS | CLLNNFYPREAKVQWKVDNALQSGNSQESVTE |
| GALTSGVHTFPAVLQSSGLYSLSSVVTVPSSNF | QDSKDSTYSLSSTLTLSKADYEKHKVYACEVTH |
| GTQTYTCNVDHKPSNTKVDKTVERKCCVECPP | QGLSSPVTKSFNRGEC (SEQ ID NO: 283) |
| CPAPPVAGPSVFLFPPKPKDTLMISRTPEVTCVV | |
| VDVSHEDPEVQFNWYVDGVEVHNAKTKPREEQ | |
| FNSTFRVVSVLTVVHQDWLNGKEYKCKVSNKG | |
| LPAPIEKTISKTKGQPREPQVYTLPPSREEMTKN | |
| QVSLTCLVKGFYPSDIAVEWESNGQPENNYKTT | |
| PPMLDSDGSFFLYSKLTVDKSRWQQGNVFSCS | |
| VMHEALHNHYTQKSLSLSPGK (SEQ ID NO: | |
| 282) | |
| QVQLVQSGAEVKKPGASVKVSCKASGYTFTGY | DIVMTQSPLSLPVTPGEPASISCRSSQSLLHSNG |
| YMHWVRQAPGQGLEWMGWINPNSGGTNYAQ | YNYLDWYLQKPGQSPQVLIYLVSNRASGVPDRF |
| KFQGRVTMTRDTSISTAYLELSRLRSDDTAVYY | SGSGSGTDFTLKISRVEAEDVGVYYCMQALQTP |
| CARNTVGTGYYYYGMDVWGQGTTVTVSSAST | PTFGGGTKVEIKRTVAAPSVFIFPPSDEQLKSGT |
| KGPSVFPLAPCSRSTSESTAALGCLVKDYFPEP | ASVVCLLNNFYPREAKVQWKVDNALQSGNSQE |
| VTVSWNSGALTSGVHTFPAVLQSSGLYSLSSVV | SVTEQDSKDSTYSLSSTLTLSKADYEKHKVYAC |
| TVPSSNFGTQTYTCNVDHKPSNTKVDKTVERK | EVTHQGLSSPVTKSFNRGEC (SEQ ID NO: 285) |
| CCVECPPCPAPPVAGPSVFLFPPKPKDTLMISR | |
| TPEVTCVVVDVSHEDPEVQFNWYVDGVEVHNA | |
| KTKPREEQFNSTFRVVSVLTVVHQDWLNGKEY | |
| KCKVSNKGLPAPIEKTISKTKGQPREPQVYTLPP | |
| SREEMTKNQVSLTCLVKGFYPSDIAVEWESNG | |
| QPENNYKTTPPMLDSDGSFFLYSKLTVDKSRW | |
| QQGNVFSCSVMHEALHNHYTQKSLSLSPGK | |
| (SEQ ID NO: 284) | |
| EVQLLESGGGLIQPGGSLRLSCAASGFTFSSFA | EIVMTQSPATLSVSPGERATLSCRASQSVSSNL |
| MSWVRQAPGKGLEWVSTISGSGGYTFYADSVK | AWYQQKPGQAPRLLIYGASTRATGFPARFSGS |
| GRFTISRDNSKNTLYLQMNSLRAEDTAVYYCAK | GSGTEFTLTISSLQSEDFAIYYCQQYNNWPLTF |
| DGRYNWNYGAFDIWGQGTMVTVSSASTKGPS | GGGTKVEIKRTVAAPSVFIFPPSDEQLKSGTASV |
| VFPLAPCSRSTSESTAALGCLVKDYFPEPVTVS | VCLLNNFYPREAKVQWKVDNALQSGNSQESVT |
| WNSGALTSGVHTFPAVLQSSGLYSLSSVVTVPS | EQDSKDSTYSLSSTLTLSKADYEKHKVYACEVT |
| SSLGTKTYTCNVDHKPSNTKVDKRVESKYGPP | HQGLSSPVTKSFNRGEC (SEQ ID NO: 287) |
| CPSCPAPEFLGGPSVFLFPPKPKDTLMISRTPEV | |
| TCVVVDVSQEDPEVQFNWYVDGVEVHNAKTKP | |
| REEQFNSTYRVVSVLTVLHQDWLNGKEYKCKV | |
| SNKGLPSSIEKTISKAKGQPREPQVYTLPPSQEE | |
| MTKNQVSLTCLVKGFYPSDIAVEWESNGQPEN | |
| NYKTTPPVLDSDGSFFLYSRLTVDKSRWQEGN | |
| VFSCSVMHEALHNHYTQKSLSLSLGK (SEQ ID | |
| NO: 286) | |
| EVQLVESGGGLVKPGGSLRLSCAASGFTFSSY | DMQMTQSPSSLSASVGDRVTITCRASQGIRNDL |
| SMNWVRQAPGKGLEWVSSISSSSSYIYYADSV | GWYQQKPGKAPKRLIYGASSLQSGVPSRFSGS |
| KGRFTISRDNAKNSLYLQMNSLRAEDTAVYYCA | GSGTEFTLTISSLQPEDSATYYCLQHNSYPLTFG |
| RDRGSYFLFDYWGQGTLVTVSSASTKGPSVFP | GGTKVEIKRTVAAPSVFIFPPSDEQLKSGTASVV |
| LAPCSRSTSESTAALGCLVKDYFPEPVTVSWNS | CLLNNFYPREAKVQWKVDNALQSGNSQESVTE |
| GALTSGVHTFPAVLQSSGLYSLSSVVTVPSSNF | QDSKDSTYSLSSTLTLSKADYEKHKVYACEVTH |
| GTQTYTCNVDHKPSNTKVDKTVERKCCVECPP | QGLSSPVTKSFNRGEC (SEQ ID NO: 289) |
| CPAPPVAGPSVFLFPPKPKDTLMISRTPEVTCVV | |
| VDVSHEDPEVQFNWYVDGVEVHNAKTKPREEQ | |
| FNSTFRVVSVLTVVHQDWLNGKEYKCKVSNKG | |
| LPAPIEKTISKTKGQPREPQVYTLPPSREEMTKN | |
| QVSLTCLVKGFYPSDIAVEWESNGQPENNYKTT | |
| PPMLDSDGSFFLYSKLTVDKSRWQQGNVFSCS | |
| VMHEALHNHYTQKSLSLSPGK (SEQ ID NO: | |
| 288) | |
| EVQLVESGGGLVKPGGSLRLSCAASGFTFSSY | DIQMTQSPSSLSASVGDRVTITCRASQGIRNDL |
| SMNWVRQAPGKGLEWVSSISSSSSYIYYADSV | GWYQQKPGKAPKRLIYAASSLQSGVPSRFSGS |
| KGRFTISRDNAKNSLYLQMNSLRAEDTAVYYCA | GSGTEFTLTISSLQPEDFATYYCLQLNSYPFTFG |
| RDRGYSYGLDYWGQGTLVTVSSASTKGPSVFP | PGTKVDIKRTVAAPSVFIFPPSDEQLKSGTASVV |
| LAPCSRSTSESTAALGCLVKDYFPEPVTVSWNS | CLLNNFYPREAKVQWKVDNALQSGNSQESVTE |
| GALTSGVHTFPAVLQSSGLYSLSSVVTVPSSNF | QDSKDSTYSLSSTLTLSKADYEKHKVYACEVTH |
| GTQTYTCNVDHKPSNTKVDKTVERKCCVECPP | QGLSSPVTKSFNRGEC (SEQ ID NO: 291) |
| CPAPPVAGPSVFLFPPKPKDTLMISRTPEVTCVV | |
| VDVSHEDPEVQFNWYVDGVEVHNAKTKPREEQ | |
| FNSTFRVVSVLTVVHQDWLNGKEYKCKVSNKG | |
| LPAPIEKTISKTKGQPREPQVYTLPPSREEMTKN | |
| QVSLTCLVKGFYPSDIAVEWESNGQPENNYKTT | |
| PPMLDSDGSFFLYSKLTVDKSRWQQGNVFSCS | |
| VMHEALHNHYTQKSLSLSPGK (SEQ ID NO: | |
| 290) | |
| EVQLLESGGGLIQPGGSLRLSCAASGFTFSSFA | EIVMTQSPATLSVSPGERATLSCRASQSVRSNL |
| MSWVRQAPGKGLEWVSTISGSGGSTYYADFVK | AWFQQKPGQAPRLLIYGASTRATGIPARFSGSG |
| GRFTISRDNSKNTLYLQMNSLRAEDTAVYYCAK | SGTEFTLTISSLQSEDFAVYYCQQYNNWPLTFG |
| DGRYNWNYGAFDIWGQGTMVTVSSASTKGPS | GGTKVEIKRTVAAPSVFIFPPSDEQLKSGTASVV |
| VFPLAPCSRSTSESTAALGCLVKDYFPEPVTVS | CLLNNFYPREAKVQWKVDNALQSGNSQESVTE |
| WNSGALTSGVHTFPAVLQSSGLYSLSSVVTVPS | QDSKDSTYSLSSTLTLSKADYEKHKVYACEVTH |
| SSLGTKTYTCNVDHKPSNTKVDKRVESKYGPP | QGLSSPVTKSFNRGEC (SEQ ID NO: 293) |
| CPSCPAPEFLGGPSVFLFPPKPKDTLMISRTPEV | |
| TCVVVDVSQEDPEVQFNWYVDGVEVHNAKTKP | |
| REEQFNSTYRVVSVLTVLHQDWLNGKEYKCKV | |
| SNKGLPSSIEKTISKAKGQPREPQVYTLPPSQEE | |
| MTKNQVSLTCLVKGFYPSDIAVEWESNGQPEN | |
| NYKTTPPVLDSDGSFFLYSRLTVDKSRWQEGN | |
| VFSCSVMHEALHNHYTQKSLSLSLGK (SEQ ID | |
| NO: 292) | |
| EVQLLESGGGLVQPGGSLRLSCAASGFTFSSY | EIVMTQSPATLSVSPGERATLSCRTSQSVSSNL |
| AMSWVRQAPGKGLEWVSAISGSGGSTFY | AWYQQKPGQAPRLLIYGASTRATGIPARFSGSG |
| ADSVTGRFTISRDNSKNTLFLQLNSLRAEDTAVY | SGTEFTLTISSLQSEDFAVYYCQQYNNWPLTFG |
| HCAKDGRFNWNYGASDIWGQGTMVTVSSAST | GGTKVEIKRTVAAPSVFIFPPSDEQLKSGTASVV |
| KGPSVFPLAPCSRSTSESTAALGCLVKDYFPEP | CLLNNFYPREAKVQWKVDNALQSGNSQESVTE |
| VTVSWNSGALTSGVHTFPAVLQSSGLYSLSSVV | QDSKDSTYSLSSTLTLSKADYEKHKVYACEVTH |
| TVPSSSLGTKTYTCNVDHKPSNTKVDKRVESKY | QGLSSPVTKSFNRGEC (SEQ ID NO: 295) |
| GPPCPSCPAPEFLGGPSVFLFPPKPKDTLMISR | |
| TPEVTCVVVDVSQEDPEVQFNWYVDGVEVHNA | |
| KTKPREEQFNSTYRVVSVLTVLHQDWLNGKEY | |
| KCKVSNKGLPSSIEKTISKAKGQPREPQVYTLPP | |
| SQEEMTKNQVSLTCLVKGFYPSDIAVEWESNG | |
| QPENNYKTTPPVLDSDGSFFLYSRLTVDKSRW | |
| QEGNVFSCSVMHEALHNHYTQKSLSLSLGK | |
| (SEQ ID NO: 294) | |
| EVQLLESGGGLVQPGGSLRLSCAASGFTFSSFA | EIVMTQSPATLSVSPGERATLSCRASQSVSSNL |
| MSWVRQAPGKGLEWVSTISGSGGYTFYADSVK | AWYQQKPGQAPRLLIYGASTRATGIPARFSGSG |
| GRFTISRDNSKNTLYLQMNSLRAEDTAVYYCAK | SGTEFTLTISSLQSEDFAVYYCQQYNNWPLTFG |
| DGRYNWNYGAFDIWGQGTMVTVSSASTKGPS | GGTKVEIKRTVAAPSVFIFPPSDEQLKSGTASVV |
| VFPLAPCSRSTSESTAALGCLVKDYFPEPVTVS | CLLNNFYPREAKVQWKVDNALQSGNSQESVTE |
| WNSGALTSGVHTFPAVLQSSGLYSLSSVVTVPS | QDSKDSTYSLSSTLTLSKADYEKHKVYACEVTH |
| SNFGTQTYTCNVDHKPSNTKVDKTVERKCCVE | QGLSSPVTKSFNRGEC (SEQ ID NO: 297) |
| CPPCPAPPVAGPSVFLFPPKPKDTLMISRTPEVT | |
| CVVVDVSHEDPEVQFNWYVDGVEVHNAKTKPR | |
| EEQFNSTFRVVSVLTVVHQDWLNGKEYKCKVS | |
| NKGLPAPIEKTISKTKGQPREPQVYTLPPSREEM | |
| TKNQVSLTCLVKGFYPSDIAVEWESNGQPENNY | |
| KTTPPMLDSDGSFFLYSKLTVDKSRWQQGNVF | |
| SCSVMHEALHNHYTQKSLSLSPGK (SEQ ID | |
| NO: 296) | |
| ASTKGPSVFPLAPSSKSTSGGTAALGCLVKDYF | RTVAAPSVFIFPPSDEQLKSGTASVVCLLNNFYP |
| PEPVTVSWNSGALTSGVHTFPAVLQSSGLYSLS | REAKVQWKVDNALQSGNSQESVTEQDSKDSTY |
| SVVTVPSSSLGTQTYICNVNHKPSNTKVDKKVE | SLSSTLTLSKADYEKHKVYACEVTHQGLSSPVT |
| PKSCDKTHTCPPCPAPEAAGAPSVFLFPPKPKD | KSFNRGEC (SEQ ID NO: 368) |
| TLMISRTPEVTCVVVDVSHEDPEVKFNWYVDGV | |
| EVHNAKTKPREEQYNSTYRVVSVLTVLHQDWL | |
| NGKEYKCKVSNKALPAPIEKTISKAKGQPREPQ | |
| VYTLPPSREEMTKNQVSLTCLVKGFYPSDIAVE | |
| WESNGQPENNYKTTPPVLDSDGSFFLYSKLTV | |
| DKSRWQQGNVFSCSVMHEALHNHYTQKSLSLS | |
| PGK (SEQ ID NO: 367) | |
| EVQLLESGGGLVQPGGSLRLSCAASGFTFSSY | DIQMTQSPSSLSASVGDRVTITCKASQDVSTAV |
| AMSWVRQAPGKGLEWVSTISSGGSYTSYPDSV | AWYQQKPGKAPKLLIYSASYRYTGVPSRFSGS |
| KGRFTISRDNSKNTLYLQMNSLRAEDTAVYYCA | GSGTDFTLTISSLQPEDFATYYCQQHYSTPWTF |
| KQDYAMNYWGQGTLVTVSSASTKGPSVFPLAP | GGGTKVEIKRTVAAPSVFIFPPSDEQLKSGTASV |
| SSKSTSGGTAALGCLVKDYFPEPVTVSWNSGA | VCLLNNFYPREAKVQWKVDNALQSGNSQESVT |
| LTSGVHTFPAVLQSSGLYSLSSVVTVPSSSLGT | EQDSKDSTYSLSSTLTLSKADYEKHKVYACEVT |
| QTYICNVNHKPSNTKVDKKVEPKSCDKTHTCPP | HQGLSSPVTKSFNRGEC (SEQ ID NO: 370) |
| CPAPEAAGAPSVFLFPPKPKDTLMISRTPEVTCV | |
| VVDVSHEDPEVKFNWYVDGVEVHNAKTKPREE | |
| QYNSTYRVVSVLTVLHQDWLNGKEYKCKVSNK | |
| ALPAPIEKTISKAKGQPREPQVYTLPPSREEMTK | |
| NQVSLTCLVKGFYPSDIAVEWESNGQPENNYKT | |
| TPPVLDSDGSFFLYSKLTVDKSRWQQGN | |
| VFSCSVMHEALHNHYTQKSLSLSPGK (SEQ ID | |
| NO: 369) | |

In some embodiments, the myostatin antibody is a bi-specific antibody that also binds to activin A. Exemplary bi-specific myostatin antibodies that may be used in the methods described herein include those described in U.S. Pat. Nos. 9,718,881, 10,526,403, 10,400,036 and 8,871,209, the disclosures of which are incorporated herein by reference. In some embodiments, the bi-specific antibody includes an activin A HCVR and LCVR from Table 1 (e.g., a HCVR sequence having at least 90% (e.g., at least 91%, 92%, 93%, 94%, 95%, 96%, 97%, 98%, 99%, or 100%) sequence identity to a HCVR sequence in Table 1, such as any one of SEQ ID NOs: 138, 140, 142, 143, 144, 146, 148, 150, 151, 172, and 174, and a LCVR sequence having at least 90% (e.g., at least 91%, 92%, 93%, 94%, 95%, 96%, 97%, 98%, 99%, or 100%) sequence identity to a LCVR sequence in Table 1, such as any one of SEQ ID NOs: 139, 141, 145, 147, 149, 173, and 175) and a myostatin HCVR and LCVR from Table 3 (e.g., a HCVR sequence having at least 90% (e.g., at least 91%, 92%, 93%, 94%, 95%, 96%, 97%, 98%, 99%, or 100%) sequence identity to a HCVR sequence in Table 3, such as any one of SEQ ID NOs: 164, 188, 201, 204-210, 222-228, 234, 250, 252, 254, 256, 258, 260, 262, 264, 266, 268, 270, 272, 298, 306, 308, 310, 312, 314, 316, 318, 320, 356, 371, 373, 387, 389, 391, 405, 407, 409, 411, 413, 415, 417, 419, 421-423, 425, 427, 429, 431, 433, 435, 437, 439, 441, 444, 446, and 448-476, and a LCVR sequence having at least 90% (e.g., at least 91%, 92%, 93%, 94%, 95%, 96%, 97%, 98%, 99%, or 100%) sequence identity to a LCVR sequence in Table 3, such as any one of SEQ ID NOs: 165, 189, 202, 203, 221, 229-233, 251, 253, 255, 257, 259, 261, 263, 265, 267, 269, 271, 273, 299, 307, 309, 311, 313, 315, 317, 319, 321, 358, 372, 374, 388, 390, 392, 406, 408, 410, 412, 414, 416, 418, 420, 424, 426, 428, 430, 432, 434, 436, 438, 440, 442, 443, 445, 447, and 477-486). In some embodiments, the bi-specific antibody includes an activin A heavy chain CDR1, CDR2, and CDR3 and a light chain CDR1, CDR2, and CDR3 from Table 2 (e.g., an activin A heavy chain CDR1, CDR2, and CDR3 and a light chain CDR1, CDR2, and CDR3 from the same row of Table 2) and a myostatin heavy chain CDR1, CDR2, and CDR3 and a light chain CDR1, CDR2, and CDR3 from Table 4 (e.g., a myostatin heavy chain CDR1, CDR2, and CDR3 and a light chain CDR1, CDR2, and CDR3 from the same row of Table 4). In some embodiments, the bi-specific antibody includes an activin A HCVR of SEQ ID NO: 138 and LCVR of SEQ ID NO: 139 and a myostatin HCVR of SEQ ID NO: 164 and LCVR of SEQ ID NO: 165. In some embodiments, the bi-specific antibody includes an activin A HCVR of SEQ ID NO: 138 and LCVR of SEQ ID NO: 139 and a myostatin HCVR of SEQ ID NO: 387 and LCVR of SEQ ID NO: 388. In some embodiments, the bi-specific antibody includes an activin A HCVR of SEQ ID NO: 138 and LCVR of SEQ ID NO: 139 and a myostatin HCVR of SEQ ID NO: 391 and LCVR of SEQ ID NO: 392. In some embodiments, the bi-specific antibody includes an activin A HCVR of SEQ ID NO: 144 and LCVR of SEQ ID NO: 145 and a myostatin HCVR of SEQ ID NO: 164 and LCVR of SEQ ID NO: 165. In some embodiments, the bi-specific antibody includes an activin A HCVR of SEQ ID NO: 144 and LCVR of SEQ ID NO: 145 and a myostatin HCVR of SEQ ID NO: 387 and LCVR of SEQ ID NO: 388. In some embodiments, the bi-specific antibody includes an activin A HCVR of SEQ ID NO: 144 and LCVR of SEQ ID NO: 145 and a myostatin HCVR of SEQ ID NO: 391 and LCVR of SEQ ID NO: 392. In some embodiments, the bi-specific antibody includes an activin A heavy chain CDR1, CDR2, and CDR3 and a light chain CDR1, CDR2, and CDR3 of SEQ ID NOs: 152-157 and a myostatin heavy chain CDR1, CDR2, and CDR3 and a light chain CDR1, CDR2, and CDR3 of SEQ ID NOs: 166-171. In some embodiments, the bi-specific antibody includes an activin A heavy chain CDR1, CDR2, and CDR3 and a light chain CDR1, CDR2, and CDR3 of SEQ ID NOs: 158-163 and a myostatin heavy chain CDR1, CDR2, and CDR3 and a light chain CDR1, CDR2, and CDR3 of SEQ ID NOs: 166-171.

In some embodiments, the ActRII signaling inhibitor is an activin B antibody or an antigen binding fragment thereof. Activin B antibodies that may be used in the methods described herein include those described in U.S. Pat. No. 8,383,351, which is incorporated herein by reference. In some embodiments, the activin B antibody or an antigen binding fragment thereof has a HCVR including three CDRs from the HCVR sequence of SEQ ID NO: 494 and a LCVR including three CDRs from the LCVR sequence of SEQ ID NO: 495. In some embodiments, the activin B antibody or an antigen binding fragment thereof has a HCVR having at least 90% (e.g., at least 91%, 92%, 93%, 94%, 95%, 96%, 97%, 98%, 99%, or 100%) sequence identity to SEQ ID NO: 494. In some embodiments, the activin B antibody or an antigen binding fragment thereof has a LCVR having at least 90% (e.g., at least 91%, 92%, 93%, 94%, 95%, 96%, 97%, 98%, 99%, or 100%) sequence identity to SEQ ID NO: 495.

(SEQ ID NO: 494)
MKCSWIMFFLVATATGVHSQVQLQQPGAELVKPGASVKLSCKASG
YTFTNYWMYWVKQRPGQGLEWIGMIHPNSGSTNYNGKFKTGATLT
VDKSSSTVYMQLSSLTSEDSAVYYCARWGYGGNYDYAMDYWGQGT
SVTVSSAKTTPPSVYPLAPGSL
(SEQ ID NO: 495)
MDFQVQIFSFLLISASVIMSRGQIVLTQSPAIMSASLGERVTMTC
TASSSVSSSYFHWYQQKPGSSPKLWIYSTSNLASGVPARFSGSGS
GTSYSLTISTMEAEDAVTYYCHQYHRSPWTFGGGTKLEIKRADAA
PTVSIFPPSSEQLTSGGASVVCFLNNFYPK

In some embodiments, the ActRII signaling inhibitor is a GDF-11 antibody or an antigen binding fragment thereof.

In some embodiments, the ActRII signaling inhibitor is ActRII antibody or an antigen binding fragment thereof. There exist two types of activin type II receptors: ActRIIA and ActRIIB. In some embodiments, the ActRII antibody is an ActRIIA antibody or an antigen binding fragment thereof. In some embodiments, the ActRII antibody is an ActRIIB antibody or an antigen binding fragment thereof. In some embodiments, the ActRII antibody or an antigen binding fragment thereof binds to both ActRIIA and ActRIIB. In some embodiments, the ActRII antibody is Bimagrumab (also known as BYM338), CSJ089, CQI876, or CDD861 (described in Morvan et al., PNAS 114:12448-12453 (2017)). Additional ActRII antibodies that may be used in the methods described herein include those described in International Patent Application Publication Nos. WO2010125003, WO2012064771, WO2017156488, WO2013063536, WO2018175460, WO2021044287, WO2013188448, and WO2020243448; US Patent Application No. US20180066061, US20180230221, US20180111991, US20200181271, US20210309749, and US20160200818; and U.S. Pat. Nos. 9,453,080, 10,266,598, 10,981,999, 10,266,598, 10,981,999, 10,307,455, 11,000,565, 10,982,000, 9,969,806, 9,365,651, 8,388,968, 8,551,482, 9,493,556, 8,765,385, and 9,624,301, each of which is incorporated herein by reference.

In some embodiments, the ActRII antibody or an antigen binding fragment thereof has a HCVR and a LCVR listed in Table 8 (e.g., an HCVR and an LCVR from the same row of Table 8). In some embodiments, the ActRII antibody or antigen binding fragment thereof includes a HCVR sequence having at least 90% (e.g., at least 91%, 92%, 93%, 94%, 95%, 96%, 97%, 98%, 99%, or 100%) sequence identity to a HCVR sequence in Table 8, such as any one of SEQ ID NOs: 512, 514, 516, 518, 520, 522, 524, 526, 528, 530, 532, 534, 536, 538, 583, 591, 593, 595-598, 600, 602, 603, 605, 606, 608, 610-614, 687, 689, 692, 695, and 697, and a LCVR sequence having at least 90% (e.g., at least 91%, 92%, 93%, 94%, 95%, 96%, 97%, 98%, 99%, or 100%) sequence identity to a LCVR sequence in Table 8, such as any one of SEQ ID NOs: 513, 515, 517, 519, 521, 523, 525, 527, 529, 531, 533, 535, 537, 539, 584, 592, 594, 601, 604, 607, 609, 615, 688, 690, 691, 693, 694, 696, and 698. In some embodiments, the ActRII antibody or an antigen binding fragment thereof, apart from the light chain CDR1, CDR2, and CDR3 and the heavy chain CDR1, CDR2, and CDR3, has a HCVR and LCVR sequence having at least 90% sequence identity (e.g., 90%, 91%, 92%, 93%, 94%, 95%, 95%, 97%, 98%, 99% or more sequence identity) to a HCVR and LCVR sequence listed in Table 8. In some embodiments, the ActRII antibody or an antigen binding fragment thereof has the light chain CDR1, CDR2, and CDR3 and the heavy chain CDR1, CDR2, and CDR3 sequences of an HCVR sequence and an LCVR sequence in Table 8. In some embodiments, the ActRII antibody or antigen binding fragment thereof includes an HCVR sequence and an LCVR sequence from the same row of Table 8.

TABLE 8
Exemplary HCVR and LCVR sequences of ActRII antibodies

| HCVR | LCVR |
|---|---|
| QVQLVQSGAEVKKPGASVKVSCKASGYTFTSS | DIALTQPASVSGSPGQSITISCTGTSSDVGSYNY |
| YINWVRQAPGQGLEWMGTINPVSGSTSYAQKF | VNWYQQHPGKAPKLMIYGVSKRPSGVSNRFSG |
| QGRVTMTRDTSISTAYMELSSLRSEDTAVYYCA | SKSGNTASLTISGLQAEDEADYYCGTFAGGSYY |
| RGGWFDYWGQGTLVTVSS (SEQ ID NO: 512) | GVFGGGTKLTVLGQ (SEQ ID NO: 513) |
| QVQLVQSGAEVKKPGASVKVSCKASGYTFTSS | DIALTQPASVSGSPGQSITISCTGTSSDVGSYNY |
| YINWVRQAPGQGLEWMGTINPVSGNTSYAQKF | VNWYQQHPGKAPKLMIYGVSKRPSGVSNRFSG |
| QGRVTMTRDTSISTAYMELSSLRSEDTAVYYCA | SKSGNTASLTISGLQAEDEADYYCQAWTSKMA |
| RGGWFDYWGQGTLVTVSS (SEQ ID NO: 514) | GVFGGGTKLTVLGQ (SEQ ID NO: 515) |
| QVQLVQSGAEVKKPGASVKVSCKASGYTFTSS | DIALTQPASVSGSPGQSITISCTGTSSDVGSYNY |
| YINWVRQAPGQGLEWMGTINPVSGNTSYAQKF | VNWYQQHPGKAPKLMIYGVSKRPSGVSNRFSG |
| QGRVTMTRDTSISTAYMELSSLRSEDTAVYYCA | SKSGNTASLTISGLQAEDEADYYCSSYTRMGHP |
| RGGWFDYWGQGTLVTVSS (SEQ ID NO: 516) | VFGGGTKLTVLGQ (SEQ ID NO: 517) |
| QVQLVQSGAEVKKPGASVKVSCKASGYTFTSS | DIALTQPASVSGSPGQSITISCTGTSSDVGSYNY |
| YINWVRQAPGQGLEWMGTINPVSGNTSYAQKF | VNWYQQHPGKAPKLMIYGVSKRPSGVSNRFSG |
| QGRVTMTRDTSISTAYMELSSLRSEDTAVYYCA | SKSGNTASLTISGLQAEDEADYYCATYGKGVTP |
| RGGWFDYWGQGTLVTVSS (SEQ ID NO: 518) | PVFGGGTKLTVLGQ (SEQ ID NO: 519) |
| QVQLVQSGAEVKKPGASVKVSCKASGYTFTSS | DIALTQPASVSGSPGQSITISCTGTSSDVGSYNY |
| YINWVRQAPGQGLEWMGTINPVSGNTSYAQKF | VNWYQQHPGKAPKLMIYGVSKRPSGVSNRFSG |
| QGRVTMTRDTSISTAYMELSSLRSEDTAVYYCA | SKSGNTASLTISGLQAEDEADYYCGTFAGGSYY |
| RGGWFDYWGQGTLVTVSS (SEQ ID NO: 520) | GVFGGGTKLTVLGQ (SEQ ID NO: 521) |
| QVQLVQSGAEVKKPGASVKVSCKASGYTFTSS | DIALTQPASVSGSPGQSITISCTGTSSDVGSYNY |
| YINWVRQAPGQGLEWMGMINAPIGTTRYAQKF | VNWYQQHPGKAPKLMIYGVSKRPSGVSNRFSG |
| QGRVTMTRDTSISTAYMELSSLRSEDTAVYYCA | SKSGNTASLTISGLQAEDEADYYCQAWTSKMA |
| RGGWFDYWGQGTLVTVSS (SEQ ID NO: 522) | GVFGGGTKLTVLGQ (SEQ ID NO: 523) |
| QVQLVQSGAEVKKPGASVKVSCKASGYTFTSS | DIALTQPASVSGSPGQSITISCTGTSSDVGSYNY |
| YINWVRQAPGQGLEWMGQINAASGMTRYAQK | VNWYQQHPGKAPKLMIYGVSKRPSGVSNRFSG |
| FQGRVTMTRDTSISTAYMELSSLRSEDTAVYYC | SKSGNTASLTISGLQAEDEADYYCQAWTSKMA |
| ARGGWFDYWGQGTLVTVSS | GVFGGGTKLTVLGQ (SEQ ID NO: 525) |
| (SEQ ID NO: 524) | |
| QVQLVQSGAEVKKPGASVKVSCKASGYTFTSS | DIALTQPASVSGSPGQSITISCTGTSSDVGSYNY |
| YINWVRQAPGQGLEWMGMINAPIGTTRYAQKF | VNWYQQHPGKAPKLMIYGVSKRPSGVSNRFSG |
| QGRVTMTRDTSISTAYMELSSLRSEDTAVYYCA | SKSGNTASLTISGLQAEDEADYYCGTFAGGSYY |
| RGGWFDYWGQGTLVTVSS (SEQ ID NO: 526) | GVFGGGTKLTVLGQ (SEQ ID NO: 527) |
| QVQLVQSGAEVKKPGASVKVSCKASGYTFTSS | DIALTQPASVSGSPGQSITISCTGTSSDVGSYNY |
| YINWVRQAPGQGLEWMGTINPVSGNTRYAQKF | VNWYQQHPGKAPKLMIYGVSKRPSGVSNRFSG |
| QGRVTMTRDTSISTAYMELSSLRSEDTAVYYCA | SKSGNTASLTISGLQAEDEADYYCGTFAGGSYY |
| RGGWFDYWGQGTLVTVSS (SEQ ID NO: 528) | GVFGGGTKLTVLGQ (SEQ ID NO: 529) |
| QVQLVQSGAEVKKPGASVKVSCKASGYTFTSS | DIALTQPASVSGSPGQSITISCTGTSSDVGSYNY |
| YINWVRQAPGQGLEWMGQINAASGMTRYAQK | VNWYQQHPGKAPKLMIYGVSKRPSGVSNRFSG |
| FQGRVTMTRDTSISTAYMELSSLRSEDTAVYYC | SKSGNTASLTISGLQAEDEADYYCGTFAGGSYY |
| ARGGWFDYWGQGTLVTVSS (SEQ ID NO: 530) | GVFGGGTKLTVLGQ (SEQ ID NO: 531) |
| QVQLVQSGAEVKKPGASVKVSCKASGYTFTSS | DIALTQPASVSGSPGQSITISCTGTSSDVGSYNY |
| YINWVRQAPGQGLEWMGNINAAAGITLYAQKF | VNWYQQHPGKAPKLMIYGVSKRPSGVSNRFSG |
| QGRVTMTRDTSISTAYMELSSLRSEDTAVYYCA | SKSGNTASLTISGLQAEDEADYYCGTFAGGSYY |
| RGGWFDYWGQGTLVTVSS (SEQ ID NO: 532) | GVFGGGTKLTVLGQ (SEQ ID NO: 533) |
| QVQLVQSGAEVKKPGASVKVSCKASGYTFTSS | DIALTQPASVSGSPGQSITISCTGTSSDVGSYNY |
| YINWVRQAPGQGLEWMGTINPPTGGTYYAQKF | VNWYQQHPGKAPKLMIYGVSKRPSGVSNRFSG |
| QGRVTMTRDTSISTAYMELSSLRSEDTAVYYCA | SKSGNTASLTISGLQAEDEADYYCGTFAGGSYY |
| RGGWFDYWGQGTLVTVSS (SEQ ID NO: 534) | GVFGGGTKLTVLGQ (SEQ ID NO: 535) |
| QVQLVQSGAEVKKPGASVKVSCKASGYTFTSS | DIALTQPASVSGSPGQSITISCTGTSSDVGSYNY |
| YINWVRQAPGQGLEWMGGINPPAGTTSYAQKF | VNWYQQHPGKAPKLMIYGVSKRPSGVSNRFSG |
| QGRVTMTRDTSISTAYMELSSLRSEDTAVYYCA | SKSGNTASLTISGLQAEDEADYYCGTFAGGSYY |
| RGGWFDYWGQGTLVTVSS (SEQ ID NO: 536) | GVFGGGTKLTVLGQ (SEQ ID NO: 537) |
| QVQLVQSGAEVKKPGASVKVSCKASGYTFTSS | DIALTQPASVSGSPGQSITISCTGTSSDVGSYNY |
| YINWVRQAPGQGLEWMGNINPATGHADYAQKF | VNWYQQHPGKAPKLMIYGVSKRPSGVSNRFSG |
| QGRVTMTRDTSISTAYMELSSLRSEDTAVYYCA | SKSGNTASLTISGLQAEDEADYYCGTFAGGSYY |
| RGGWFDYWGQGTLVTVSS (SEQ ID NO: 538) | GVFGGGTKLTVLGQ (SEQ ID NO: 539) |
| DVQLQESGPGLVKPSQSLSLTCSVTGYSITSGY | DIQMTQTTSSLSASLGDRVTISCRASQDISNFLN |
| YWNWIRQFPGNKLEWMGYISYDGSNNYNPSLI | WYQQKPDGTVKLLIYFTSRLHSGVPSRFSGSGS |
| NRISITRDTSKNQFFLKLNSVTTEDTATYFCASY | GTDYSLTITNLEQEDIATYFCQQGNTLPWTFGG |
| AYRNDVRFAYWGQGTLVTVSA (SEQ ID NO: | GTKLEIK (SEQ ID NO: 584) |
| 583) | |
| QVQLVQSGAEVKKPGASVKVSCKASGYTFTSY | DIQMTQSPSSVSASVGDRVTITCRASQGISRWL |
| RMHWVRQAPGQGLEWMGFIVPSGGSTGYAQK | AWYQQKPGKAPKLLIYAASSLQSGVPSRFSGS |
| FQGRVTMTRDTSTSTVYMELSSLRSEDTAVYYC | GSGTDFTLTISSLQPEDFATYYCQQAFSHPWTF |
| ARVSRYAPEPMDVWGQGTTVTVSS (SEQ ID | GGGTKVEIK (SEQ ID NO: 592) |
| NO: 591) | |
| QLQLQESGPGLVKPSETLSLTCTVSGGSISSSS | EIVLTQSPATLSVSPGERATLSCRASQSVGSNL |
| YAWGWIRQPPGKGLEWIGSIYYSGSTYYNPSLK | AWYQQKPGQAPRLLIYGASTRATGIPARFSGSG |
| SRVTISVDTSKNQFSLKLSSVTAADTAVYYCARD | SGTEFTLTISSLQSEDFAVYYCQQYFHFPLTFGG |
| SGIGYSYASSHGYYYYMDVWGKGTTVTVSS | GTKVEIK (SEQ ID NO: 594) |
| (SEQ ID NO: 593) | |
| QVQLQESGPGLVKPSQTLSLTCTVSGGSIGSGG | EIVLTQSPATLSVSPGERATLSCRASQSVGSNL |
| YYWSWIRQHPGKGLEWIGGIYGSGSTYYNPSL | AWYQQKPGQAPRLLIYGASTRATGIPARFSGSG |
| KSRVTISVDTSKNQFSLKLSSVTAADTAVYYCAR | SGTEFTLTISSLQSEDFAVYYCQQYFHFPLTFGG |
| DSGIGYSYASSHGYYYYMDVWGKGTTVTVSS | GTKVEIK (SEQ ID NO: 594) |
| (SEQ ID NO: 595) | |
| QVQLQESGPGLVKPSQTLSLTCTVSGGSIKSGG | EIVLTQSPATLSVSPGERATLSCRASQSVGSNL |
| YYWSWIRQHPGKGLEWIGGIYPSGSTYYNPSLK | AWYQQKPGQAPRLLIYGASTRATGIPARFSGSG |
| SRVTISVDTSKNQFSLKLSSVTAADTAVYYCARD | SGTEFTLTISSLQSEDFAVYYCQQYFHFPLTFGG |
| SGIGYSYASSHGYYYYMDVWGKGTTVTVSS | GTKVEIK (SEQ ID NO: 594) |
| (SEQ ID NO: 596) | |
| QVQLQESGPGLVKPSQTLSLTCTVSGGSILSGG | EIVLTQSPATLSVSPGERATLSCRASQSVGSNL |
| YYWSWIRQHPGKGLEWIGGIYYSGKTYYNPSLK | AWYQQKPGQAPRLLIYGASTRATGIPARFSGSG |
| SRVTISVDTSKNQFSLKLSSVTAADTAVYYCARD | SGTEFTLTISSLQSEDFAVYYCQQYFHFPLTFGG |
| SGIGYSYASSHGYYYYMDVWGKGTTVTVSS | GTKVEIK (SEQ ID NO: 594) |
| (SEQ ID NO: 597) | |
| QVQLQESGPGLVKPSQTLSLTCTVSGGSISSGG | EIVLTQSPATLSVSPGERATLSCRASQSVGSNL |
| YFWSWIRQHPGKGLEWIGGIYYSGRTYYNPSLK | AWYQQKPGQAPRLLIYGASTRATGIPARFSGSG |
| SRVTISVDTSKNQFSLKLSSVTAADTAVYYCARD | SGTEFTLTISSLQSEDFAVYYCQQYFHFPLTFGG |
| SGIGYSYASSHGYYYYMDVWGKGTTVTVSS | GTKVEIK (SEQ ID NO: 594) |
| (SEQ ID NO: 598) | |
| QVQLQESGPGLVKPSQTLSLTCTVSGGSIESGG | EIVLTQSPATLSVSPGERATLSCRASQSVGSNL |
| YYWSWIRQHPGKGLEWIGGIYGSGSTYYNPSL | AWYQQKPGQAPRLLIYGASTRATGIPARFSGSG |
| KSRVTISVDTSKNQFSLKLSSVTAADTAVYYCAR | SGTEFTLTISSLQSEDFAVYYCQQYFHFPLTFGG |
| DSGIGYSYASSHGYYYYMDVWGKGTTVTVSS | GTKVEIK (SEQ ID NO: 594) |
| (SEQ ID NO: 599) | |
| QLQLQESGPGLVKPSETLSLTCTVSGGSISSSS | DIQMTQSPSSVSASVGDRVTITCRASQGISSWL |
| YAWGWIRQPPGKGLEWIGSIYYSGSTYYNPSLK | AWYQQKPGKAPKLLIYAASNLQSGVPSRFSGS |
| SRVTISVDTSKNQFSLKLSSVTAADTAVYYCARA | GSGTDFTLTISSLQPEDFATYYCQQAPDLPITFG |
| GKYRWHGMDVWGQGTTVTVSS (SEQ ID NO: | GGTKVEIK (SEQ ID NO: 601) |
| 600) | |
| QVQLQESGPGLVKPSETLSLTCAVSGYSISSGV | DIQMTQSPSSVSASVGDRVTITCRASQGISSWL |
| YWMWIRQPPGKGLEWIGSIVHSGHTYYNPSLK | AWYQQKPGKAPKLLIYAASNLQSGVPSRFSGS |
| SRVTISVDTSKNQFSLKLSSVTAADTAVYYCARA | GSGTDFTLTISSLQPEDFATYYCQQAPDLPITFG |
| GKYRWHGMDVWGQGTTVTVSS (SEQ ID NO: | GGTKVEIK (SEQ ID NO: 601) |
| 602) | |
| EVQLLESGGGLVQPGGSLRLSCAASGFTFSSY | DIQMTQSPSTLSASVGDRVTITCRASQSISSWLA |
| AMSWVRQAPGKGLEWVSGISGSGGSTYYADS | WYQQKPGKAPKLLIYDASSLESGVPSRFSGSGS |
| VKGRFTISRDNSKNTLYLQMNSLRAEDTAVYYC | GTEFTLTISSLQPDDFATYYCQQYNRHSPTFGG |
| AKDPLSLLLGYFDYWGQGALVTVSS (SEQ ID | GTKVEIK (SEQ ID NO: 604) |
| NO: 603) | |
| EVQLLESGGGLVQPGGSLRLSCAASGFTFSRY | DIQMTQSPSTLSASVGDRVTITCRASQSISSWLA |
| AMSWVRQAPGKGLEWVSGISGSGGATYYADS | WYQQKPGKAPKLLIYDASSLESGVPSRFSGSGS |
| VKGRFTISRDNSKNTLYLQMNSLRAEDTAVYYC | GTEFTLTISSLQPDDFATYYCQQYNRHSPTFGG |
| AKDPLSLLLGYFDYWGQGALVTVSS (SEQ ID | GTKVEIK (SEQ ID NO: 604) |
| NO: 605) | |
| EVQLLESGGGLVQPGGSLRLSCAASGFTFGSY | DIQMTQSPSSVSASVGDRVTITCRASQGISSWL |
| GMTWVRQAPGKGLEWVSVISGSGGGTYYADS | AWYQQKPGKAPKLLIYAASSLQSGVPSRFSGS |
| VKGRFTISRDNSKNTLYLQMNSLRAEDTAVYYC | GSGTDFTLTISSLQPEDFATYYCQQVFSYPLTFG |
| AKGPRIVGMDVWGQGTTVTVS (SEQ ID NO: | GGTKVEIK (SEQ ID NO: 607) |
| 606) | |
| QVQLVQSGAEVKKPGASVKVSCKASGYTFTSY | DIQMTQSPSSVSASVGDRVTITCRASQGISRWL |
| GISWVRQAPGQGLEWMGWISPYNGNTNYAQK | AWYQQKPGKAPKLLIYAASSLQSGVPSRFSGS |
| LQGRVTMTTDTSTSTAYMELRSLRSDDTAVYYC | GSGTDFTLTISSLQPEDFATYYCQQAFSHPWTF |
| ARVSMYAPEPMDVWGQGTTVTVSS (SEQ ID | GGGTKVEIK (SEQ ID NO: 609) |
| NO: 608) | |
| QVQLVQSGAEVKKPGASVKVSCKASGYTFTGH | DIQMTQSPSSVSASVGDRVTITCRASQGISRWL |
| KMHWVRQAPGQGLEWMGWINPASGWTNYAQ | AWYQQKPGKAPKLLIYAASSLQSGVPSRFSGS |
| KFQGRVTMTRDTSISTAYMELSRLRSDDTAVYY | GSGTDFTLTISSLQPEDFATYYCQQAFSHPWTF |
| CARVSMYAPEPMDVWGQGTTVTVSS (SEQ ID | GGGTKVEIK (SEQ ID NO: 609) |
| NO: 610) | |
| QVQLVQSGAEVKKPGASVKVSCKASGYTFTSY | DIQMTQSPSSVSASVGDRVTITCRASQGISRWL |
| NMAWVRQAPGQGLEWMGIIRPSVGSTSYAQKF | AWYQQKPGKAPKLLIYAASSLQSGVPSRFSGS |
| QGRVTMTRDTSTSTVYMELSSLRSEDTAVYYC | GSGTDFTLTISSLQPEDFATYYCQQAFSHPWTF |
| ARVSMYAPEPMDVWGQGTTVTVSS (SEQ ID | GGGTKVEIK (SEQ ID NO: 609) |
| NO: 611) | |
| QVQLVQSGAEVKKPGASVKVSCKASGYTFTSY | DIQMTQSPSSVSASVGDRVTITCRASQGISRWL |
| RMHWVRQAPGQGLEWMGFIVPSGGSTSYAQK | AWYQQKPGKAPKLLIYAASSLQSGVPSRFSGS |
| FQGRVTMTRDTSTSTVYMELSSLRSEDTAVYYC | GSGTDFTLTISSLQPEDFATYYCQQAFSHPWTF |
| ARVSMYAPEPMDVWGQGTTVTVSS (SEQ ID | GGGTKVEIK (SEQ ID NO: 609) |
| NO: 612) | |
| QVQLVQSGAEVKKPGASVKVSCKASGYTFTSY | DIQMTQSPSSVSASVGDRVTITCRASQGISRWL |
| RMHWVRQAPGQGLEWMGFIVPSGGSTGYAQK | AWYQQKPGKAPKLLIYAASSLQSGVPSRFSGS |
| FQGRVTMTRDTSTSTVYMELSSLRSEDTAVYYC | GSGTDFTLTISSLQPEDFATYYCQQAFSHPWTF |
| ARVSRYAPEPMDVWGQGTTVTVSS (SEQ ID | GGGTKVEIK (SEQ ID NO: 609) |
| NO: 613) | |
| QVQLQESGPGLVKPSQTLSLTCTVSGGSISSGS | EIVLTQSPATLSLSPGERATLSCRASQSVSSYLA |
| YYWSWIRQHPGKGLEWIGYIYYSGSTYYNPSLK | WYQQKPGQAPRLLIYDASNRATGIPARFSGSGS |
| SRVTISVDTSKNQFSLKLSSVTAADTAVYYCAR | GTDFTLTISSLEPEDFAVYYCQQYFHWPPTFGG |
| GLGMYYHVPFDIWGQGTMVTVSS (SEQ ID NO: | GTKVEIK (SEQ ID NO: 615) |
| 614) | |
| QVQLVQSGAEVKKPGASVKVSCKASGYTFTSS | QSALTQPASVSGSPGQSITISCTGTSSDVGSYN |
| YINWVRQAPGQGLEWMGTINPVSGSTSYAQKF | YVNWYQQHPGKAPKLMIYGVSKRPSGVSNRFS |
| QGRVTMTRDTSISTAYMELSRLRSDDTAVYYCA | GSKSGNTASLTISGLQAEDEADYYCGTFAGGSY |
| RGGWFDYWGQGTLVTVSS (SEQ ID NO: 687) | YGVFGGGTKLTVL (SEQ ID NO: 688) |
| QLQLQESGPGLVKPSETLSLTCTVSGGSISSSS | DIVMTQSPDSLAVSLGERATINCKSSQSVLYSSN |
| YYWGWIRQPPGKGLEWIGSISYSGSTYYNPSLK | NKNYLAWYQQKPGQPPKLLIYWASTRESGVPD |
| SRVTISVDTSKNQFSLKLSSVTAADTAVYYCARD | RFSGSGSGTDFTLTISSLQAEDVAVYYCQQYAL |
| SLRYGMDVWGQGTTVTVSS (SEQ ID NO: 689) | APPRTFGGGTKVEIK (SEQ ID NO: 690) |
| QLQLQESGPGLVKPSETLSLTCTVSGGSISSSS | EIVMTQSPATLSVSPGERATLSCRASQSVSSNL |
| YYWGWIRQPPGKGLEWIGSISYSGSTYYNPSLK | AWYQQKPGQAPRLLIYGASTRATGIPARFSGSG |
| SRVTISVDTSKNQFSLKLSSVTAADTAVYYCARD | SGTEFTLTISSLQSEDFAVYYCQVYNVWPRTFG |
| SLRYGMDVWGQGTTVTVSS (SEQ ID NO: 689) | GGTKVEIK (SEQ ID NO: 691) |
| EVQLLESGGGLVQPGGSLRLSCAASGFTFGSY | DIQMTQSPSSVSASVGDRVTITCRASQGISSWL |
| GMTWVRQAPGKGLEWVSVISGSGGGTYYADS | AWYQQKPGKAPKLLIYAASSLQSGVPSRFSGS |
| VKGRFTISRDNSKNTLYLQMNSLRAEDTAVYYC | GSGTDYTLTISSLQPEDFATYYCQQVFSYPLTF |
| AKGPRVVGMDVWGQGTTVTVSS (SEQ ID NO: | GGGTKVEIK (SEQ ID NO: 693) |
| 692) | |
| QLQLQESGPGLVKPSETLSLTCTVSGGSISSSS | DIQMTQSPSTLSASVGDRVTITCRASQSIGSWL |
| YYWGWIRQPPGKGLEWIGSISYSGSTYYNPSLK | AWYQQKPGKAPKLLIYKASSLESGVPSRFSGSG |
| SRVTISVDTSKNQFSLKLSSVTAADTAVYYCARD | SGTEFTLTISSLQPDDFATYYCQVYGSYSPRTF |
| SLRYGMDVWGQGTTVTVSS (SEQ ID NO: 689) | GGGTKVEIK (SEQ ID NO: 694) |
| EVQLLESGGGLVQPGGSLRLSCAASGFTFSSY | DIQMTQSPSSVSASVGDRVTITCRASQGISSWL |
| AMSWVRQAPGKGLEWVSAISGSGGSTSYADSV | AWYQQKPGKAPKLLIYAASSLQSGVPSRFSGS |
| KGRFTISRDNSKNTLYLQMNSLRAEDTAVYYCA | GSGTDFTLTISSLQPEDFATYYCQQGHSFPLTF |
| RLPQYSRPFDYWGQGTLVTVSS (SEQ ID NO: | GGGTKVEIK (SEQ ID NO: 696) |
| 695) | |
| QVQLVQSGAEVKKPGASVKVSCKASGYTFTSY | DIQMTQSPSSVSASVGDRVTITCRASQGISRWL |
| RMHWVRQAPGQGLEWMGFIVPSGGSTSYAQK | AWYQQKPGKAPKLLIYAASSLQSGVPSRFSGS |
| FQGRVTMTRDTSTSTVYMELSSLRSEDTAVYYC | GSGTDFTLTISSLQPEDFATYYCQQAFSHPWTF |
| ARVSRYAPEPMDVWGQGTTVTVSS (SEQ ID | GGGTKVEIK (SEQ ID NO: 698) |
| NO: 697) | |

In some embodiments, the ActRII antibody or an antigen-binding fragment thereof, has the CDR sequences described in Table 9 (i.e., a light chain CDR1, CDR2, and CDR3 and a heavy chain CDR1, CDR2, and CDR3). In some embodiments, the ActRII antibody or antigen binding fragment thereof includes a light chain variable CDR1 sequence having at least 90% (e.g., at least 91%, 92%, 93%, 94%, 95%, 96%, 97%, 98%, 99%, or 100%) sequence identity to a light chain variable CDR1 sequence in Table 9, such as any one of SEQ ID NOs: 499, 505, 543, 580, 588, 619, 625, 633, 640, 648, 654, 663, 684, 702, 705, 711, 714, 720, and 726; a light chain variable CDR2 sequence having at least 90% (e.g., at least 91%, 92%, 93%, 94%, 95%, 96%, 97%, 98%, 99%, or 100%) sequence identity to a light chain variable CDR2 sequence in Table 9, such as any one of SEQ ID NOs: 500, 506, 544, 581, 589, 620, 626, 634, 641, 649, 655, 664, 685, 703, 706, 712, 715, 721, and 727; a light chain variable CDR3 sequence having at least 90% (e.g., at least 91%, 92%, 93%, 94%, 95%, 96%, 97%, 98%, 99%, or 100%) sequence identity to a light chain variable CDR3 sequence in Table 9, such as any one of SEQ ID NOs: 501, 507, 545, 547, 548, 549, 582, 590, 621, 627, 635, 635, 642, 650, 656, 665, 686, 704, 707, 713, 716, 722, and 728; a heavy chain variable CDR1 sequence having at least 90% (e.g., at least 91%, 92%, 93%, 94%, 95%, 96%, 97%, 98%, 99%, or 100%) sequence identity to a heavy chain variable CDR1 sequence in Table 9, such as any one of SEQ ID NOs: 496, 502, 540, 577, 585, 616, 622, 629, 630, 638, 644, 651, 659, 660, 669-672, 679-681, 699, 708, 717, and 723; a heavy chain variable CDR2 sequence having at least 90% (e.g., at least 91%, 92%, 93%, 94%, 95%, 96%, 97%, 98%, 99%, or more) sequence identity to a heavy chain variable CDR2 sequence in Table 9, such as any one of SEQ ID NOs: 497, 503, 541, 546, 550-556, 578, 586, 617, 623, 628, 631, 637, 643, 646, 652, 658, 661, 666, 667, 668, 676, 677, 678, 682, 700, 709, 718, and 724; and a heavy chain variable CDR3 sequence having at least 90% (e.g., at least 91%, 92%, 93%, 94%, 95%, 96%, 97%, 98%, 99%, or more) sequence identity to a heavy chain variable CDR3 sequence in Table 9, such as any one of SEQ ID NOs: 498, 504, 542, 579, 587, 618, 624, 632, 636, 639, 647, 653, 657, 662, 673, 674, 675, 683, 701, 710, 719, and 725. In some embodiments, the ActRII antibody or antigen binding fragment thereof includes a light chain CDR1, CDR2, and CDR3 sequence and a heavy chain CDR1, CDR2, and CDR3 sequence from the same row of Table 9.

TABLE 9
Exemplary CDR sequences of ActRII antibodies

| HCDR1 | HCDR2 | HCDR3 | LCDR1 | LCDR2 | LCDR3 |
|---|---|---|---|---|---|
| NSAMS (SEQ | GISVTGGSTF | VYYYSFFDY | GFNSGSVST | NTNTRSS | VLWMGSGIW |
| ID NO: 496) | YTDSVKGR | (SEQ ID NO: | SYWPS (SEQ | (SEQ ID NO: | V (SEQ ID |
| | (SEQ ID NO: | 498) | ID NO: 499) | 500) | NO: 501) |
| | 497) | | | | |
| NYAMS (SEQ | GISVTGGSTY | VYYSSFFDY | GFNSGSVST | NTNTRSS | VLYMGSGIW |
| ID NO: 502) | YTDSVKGR | (SEQ ID NO: | SYYPS (SEQ | (SEQ ID NO: | V (SEQ ID |
| | (SEQ ID NO: | 504) | ID NO: 505) | 506) | NO: 507) |
| | 503) | | | | |
| GYTFTSSYIN | TINPVSGSTS | GGWFDY | TGTSSDVGS | LMIYGVSKRP | GTFAGGSYY |
| (SEQ ID NO: | YAQKFQG | (SEQ ID NO: | YNYVN (SEQ | S (SEQ ID | G (SEQ ID |
| 540) | (SEQ ID NO: | 542) | ID NO: 543) | NO: 544) | NO: 545) |
| | 541) | | | | |
| GYTFTSSYIN | TINPVSGNTS | GGWFDY | TGTSSDVGS | LMIYGVSKRP | QAWTSKMAG |
| (SEQ ID NO: | YAQKFQG | (SEQ ID NO: | YNYVN (SEQ | S (SEQ ID | (SEQ ID NO: |
| 540) | (SEQ ID NO: | 542) | ID NO: 543) | NO: 544) | 547) |
| | 546) | | | | |
| GYTFTSSYIN | TINPVSGNTS | GGWFDY | TGTSSDVGS | LMIYGVSKRP | SSYTRMGHP |
| (SEQ ID NO: | YAQKFQG | (SEQ ID NO: | YNYVN (SEQ | S (SEQ ID | (SEQ ID NO: |
| 540) | (SEQ ID NO: | 542) | ID NO: 543) | NO: 544) | 548) |
| | 546) | | | | |
| GYTFTSSYIN | TINPVSGNTS | GGWFDY | TGTSSDVGS | LMIYGVSKRP | ATYGKGVTP |
| (SEQ ID NO: | YAQKFQG | (SEQ ID NO: | YNYVN (SEQ | S (SEQ ID | P (SEQ ID |
| 540) | (SEQ ID NO: | 542) | ID NO: 543) | NO: 544) | NO: 549) |
| | 546) | | | | |
| GYTFTSSYIN | TINPVSGNTS | GGWFDY | TGTSSDVGS | LMIYGVSKRP | GTFAGGSYY |
| (SEQ ID NO: | YAQKFQG | (SEQ ID NO: | YNYVN (SEQ | S (SEQ ID | G (SEQ ID |
| 540) | (SEQ ID NO: | 542) | ID NO: 543) | NO: 544) | NO: 545) |
| | 546) | | | | |
| GYTFTSSYIN | MINAPIGTTR | GGWFDY | TGTSSDVGS | LMIYGVSKRP | QAWTSKMAG |
| (SEQ ID NO: | YAQKFQG | (SEQ ID NO: | YNYVN (SEQ | S (SEQ ID | (SEQ ID NO: |
| 540) | (SEQ ID NO: | 542) | ID NO: 543) | NO: 544) | 547) |
| | 550) | | | | |
| GYTFTSSYIN | QINAASGMT | GGWFDY | TGTSSDVGS | LMIYGVSKRP | QAWTSKMAG |
| (SEQ ID NO: | RYAQKFQG | (SEQ ID NO: | YNYVN (SEQ | S (SEQ ID | (SEQ ID NO: |
| 540) | (SEQ ID NO: | 542) | ID NO: 543) | NO: 544) | 547) |
| | 551) | | | | |
| GYTFTSSYIN | MINAPIGTTR | GGWFDY | TGTSSDVGS | LMIYGVSKRP | GTFAGGSYY |
| (SEQ ID NO: | YAQKFQG | (SEQ ID NO: | YNYVN (SEQ | S (SEQ ID | G (SEQ ID |
| 540) | (SEQ ID NO: | 542) | ID NO: 543) | NO: 544) | NO: 545) |
| | 550) | | | | |
| GYTFTSSYIN | TINPVSGNTR | GGWFDY | TGTSSDVGS | LMIYGVSKRP | GTFAGGSYY |
| (SEQ ID NO: | YAQKFQG | (SEQ ID NO: | YNYVN (SEQ | S (SEQ ID | G (SEQ ID |
| 540) | (SEQ ID NO: | 542) | ID NO: 543) | NO: 544) | NO: 545) |
| | 552) | | | | |
| GYTFTSSYIN | QINAASGMT | GGWFDY | TGTSSDVGS | LMIYGVSKRP | GTFAGGSYY |
| (SEQ ID NO: | RYAQKFQG | (SEQ ID NO: | YNYVN (SEQ | S (SEQ ID | G (SEQ ID |
| 540) | (SEQ ID NO: | 542) | ID NO: 543) | NO: 544) | NO: 545) |
| | 551) | | | | |
| GYTFTSSYIN | NINAAAGITLY | GGWFDY | TGTSSDVGS | LMIYGVSKRP | GTFAGGSYY |
| (SEQ ID NO: | AQKFQG | (SEQ ID NO: | YNYVN (SEQ | S (SEQ ID | G (SEQ ID |
| 540) | (SEQ ID NO: | 542) | ID NO: 543) | NO: 544) | NO: 545) |
| | 553) | | | | |
| GYTFTSSYIN | TINPPTGGTY | GGWFDY | TGTSSDVGS | LMIYGVSKRP | GTFAGGSYY |
| (SEQ ID NO: | YAQKFQG | (SEQ ID NO: | YNYVN (SEQ | S (SEQ ID | G (SEQ ID |
| 540) | (SEQ ID NO: | 542) | ID NO: 543) | NO: 544) | NO: 545) |
| | 554) | | | | |
| GYTFTSSYIN | GINPPAGTTS | GGWFDY | TGTSSDVGS | LMIYGVSKRP | GTFAGGSYY |
| (SEQ ID NO: | YAQKFQG | (SEQ ID NO: | YNYVN (SEQ | S (SEQ ID | G (SEQ ID |
| 540) | (SEQ ID NO: | 542) | ID NO: 543) | NO: 544) | NO: 545) |
| | 555) | | | | |
| GYTFTSSYIN | NINPATGHAD | GGWFDY | TGTSSDVGS | LMIYGVSKRP | GTFAGGSYY |
| (SEQ ID NO: | YAQKFQG | (SEQ ID NO: | YNYVN (SEQ | S (SEQ ID | G (SEQ ID |
| 540) | (SEQ ID NO: | 542) | ID NO: 543) | NO: 544) | NO: 545) |
| | 556) | | | | |
| NYWMG (SEQ | YIRSSGGLTH | GLYSFDY | RASQGVNNF | AASTLQS | QQSYSTPRG |
| ID NO: 577) | YADSVKG | (SEQ ID NO: | LA (SEQ ID | (SEQ ID NO: | (SEQ ID NO: |
| | (SEQ ID NO: | 579) | NO: 580) | 581) | 582) |
| | 578) | | | | |
| YSITSGYYWN | YISYDGSNNY | YAYRNDVRF | RASQDISNFL | FTSRLHS | QQGNTLPWT |
| (SEQ ID NO: | NPSLIN (SEQ | AY (SEQ ID | N (SEQ ID | (SEQ ID NO: | (SEQ ID NO: |
| 585) | ID NO: 586) | NO: 587) | NO: 588) | 589) | 590) |
| SYRMH (SEQ | FIVPSGGSTG | VSRYAPEPM | RASQGISRW | AASSLQS | QQAFSHPWT |
| ID NO: 616) | YAQKFQG | DV (SEQ ID | LA (SEQ ID | (SEQ ID NO: | (SEQ ID NO: |
| | (SEQ ID NO: | NO: 618) | NO: 619) | 620) | 621) |
| | 617) | | | | |
| GGSISSSSY | YYSGS (SEQ | DSGIGYSYAS | RASQSVGSN | GASTRAT | QQYFHFPLT |
| (SEQ ID NO: | ID NO: 623) | SHGYYYYMD | LA (SEQ ID | (SEQ ID NO: | (SEQ ID NO: |
| 622) | | V (SEQ ID | NO: 625) | 626) | 627) |
| | | NO: 624) | | | |
| GGSISSSSY | YGSG (SEQ | DSGIGYSYAS | RASQSVGSN | GASTRAT | QQYFHFPLT |
| (SEQ ID NO: | ID NO: 628) | SHGYYYYMD | LA (SEQ ID | (SEQ ID NO: | (SEQ ID NO: |
| 622) | | V (SEQ ID | NO: 625) | 626) | 627) |
| | | NO: 624) | | | |
| GGSIGSGGY | YYSGS (SEQ | DSGIGYSYAS | RASQSVGSN | GASTRAT | QQYFHFPLT |
| (SEQ ID NO: | ID NO: 623) | SHGYYYYMD | LA (SEQ ID | (SEQ ID NO: | (SEQ ID NO: |
| 629) | | V (SEQ ID | NO: 625) | 626) | 627) |
| | | NO: 624) | | | |
| GGSIGSGGY | YGSG (SEQ | DSGIGYSYAS | RASQSVGSN | GASTRAT | QQYFHFPLT |
| (SEQ ID NO: | ID NO: 628) | SHGYYYYMD | LA (SEQ ID | (SEQ ID NO: | (SEQ ID NO: |
| 629) | | V (SEQ ID | NO: 625) | 626) | 627) |
| | | NO: 624) | | | |
| GFTFSSY | SGSGGS | DPLSLLLGYF | RASQSISSWL | DASSLES | QQYNRHSPT |
| (SEQ ID NO: | (SEQ ID NO: | DY (SEQ ID | A (SEQ ID | (SEQ ID NO: | (SEQ ID NO: |
| 630) | 631) | NO: 632) | NO: 633) | 634) | 635) |
| GFTFSSY | SGSGGS | DPLSLLLGYF | RASQSISSWL | DASSLES | QQYNRHSPT |
| (SEQ ID NO: | (SEQ ID NO: | DY (SEQ ID | A (SEQ ID | (SEQ ID NO: | (SEQ ID NO: |
| 630) | 631) | NO: 636) | NO: 633) | 634) | 635) |
| GFTFSSY | SGSGGA | DPLSLLLGYF | RASQSISSWL | DASSLES | QQYNRHSPT |
| (SEQ ID NO: | (SEQ ID NO: | DY (SEQ ID | A (SEQ ID | (SEQ ID NO: | (SEQ ID NO: |
| 630) | 637) | NO: 632) | NO: 633) | 634) | 635) |
| GFTFSSY | SGSGGA | DPLSLLLGYF | RASQSISSWL | DASSLES | QQYNRHSPT |
| (SEQ ID NO: | (SEQ ID NO: | DY (SEQ ID | A (SEQ ID | (SEQ ID NO: | (SEQ ID NO: |
| 630) | 637) | NO: 636) | NO: 633) | 634) | 635) |
| GFTFSRY | SGSGGS | DPLSLLLGYF | RASQSISSWL | DASSLES | QQYNRHSPT |
| (SEQ ID NO: | (SEQ ID NO: | DY (SEQ ID | A (SEQ ID | (SEQ ID NO: | (SEQ ID NO: |
| 638) | 631) | NO: 632) | NO: 633) | 634) | 635) |
| GFTFSRY | SGSGGS | DPLSLLLGYF | RASQSISSWL | DASSLES | QQYNRHSPT |
| (SEQ ID NO: | (SEQ ID NO: | DY (SEQ ID | A (SEQ ID | (SEQ ID NO: | (SEQ ID NO: |
| 638) | 631) | NO: 636) | NO: 633) | 634) | 635) |
| GFTFSRY | SGSGGA | DPLSLLLGYF | RASQSISSWL | DASSLES | QQYNRHSPT |
| (SEQ ID NO: | (SEQ ID NO: | DY (SEQ ID | A (SEQ ID | (SEQ ID NO: | (SEQ ID NO: |
| 638) | 637) | NO: 632) | NO: 633) | 634) | 635) |
| GFTFSRY | SGSGGA | DPLSLLLGYF | RASQSISSWL | DASSLES | QQYNRHSPT |
| (SEQ ID NO: | (SEQ ID NO: | DY (SEQ ID | A (SEQ ID | (SEQ ID NO: | (SEQ ID NO: |
| 638) | 637) | NO: 636) | NO: 633) | 634) | 635) |
| GGSISSSSY | YYSGS (SEQ | AGKYRWHG | RASQGISSW | AASNLQS | QQAPDLPIT |
| (SEQ ID NO: | ID NO: 623) | MDV (SEQ ID | LA (SEQ ID | (SEQ ID NO: | (SEQ ID NO: |
| 622) | | NO: 639) | NO: 640) | 641) | 642) |
| GGSISSSSY | VHSGH (SEQ | AGKYRWHG | RASQGISSW | AASNLQS | QQAPDLPIT |
| (SEQ ID NO: | ID NO: 643) | MDV (SEQ ID | LA (SEQ ID | (SEQ ID NO: | (SEQ ID NO: |
| 622) | | NO: 639) | NO: 640) | 641) | 642) |
| GYSISSGV | YYSGS (SEQ | AGKYRWHG | RASQGISSW | AASNLQS | QQAPDLPIT |
| (SEQ ID NO: | ID NO: 623) | MDV (SEQ ID | LA (SEQ ID | (SEQ ID NO: | (SEQ ID NO: |
| 644) | | NO: 639) | NO: 640) | 641) | 642) |
| GYSISSGV | VHSGH (SEQ | AGKYRWHG | RASQGISSW | AASNLQS | QQAPDLPIT |
| (SEQ ID NO: | ID NO: 643) | MDV (SEQ ID | LA (SEQ ID | (SEQ ID NO: | (SEQ ID NO: |
| 644) | | NO: 639) | NO: 640) | 641) | 642) |
| SYGMT (SEQ | VISGSGGGTY | GPRIVGMDV | RASQGISSW | AASSLQS | QQVFSYPLT |
| ID NO: 645) | YADSVKG | (SEQ ID NO: | LA (SEQ ID | (SEQ ID NO: | (SEQ ID NO: |
| | (SEQ ID NO: | 647) | NO: 648) | 649) | 650) |
| | 646) | | | | |
| GYTFTSY | SPYNGN | VSMYAPEPM | RASQGISRW | ASSLQS | QQAFSHPWT |
| (SEQ ID NO: | (SEQ ID NO: | DV (SEQ ID | LA (SEQ ID | (SEQ ID NO: | (SEQ ID NO: |
| 651) | 652) | NO: 653) | NO: 654) | 655) | 656) |
| GYTFTSY | SPYNGN | VSMYAPEPM | RASQGISRW | ASSLQS | QQAFSHPWT |
| (SEQ ID NO: | (SEQ ID NO: | DV (SEQ ID | LA (SEQ ID | (SEQ ID NO: | (SEQ ID NO: |
| 651) | 652) | NO: 657) | NO: 654) | 655) | 656) |
| GYTFTSY | NPASGW | VSMYAPEPM | RASQGISRW | ASSLQS | QQAFSHPWT |
| (SEQ ID NO: | (SEQ ID NO: | DV (SEQ ID | LA (SEQ ID | (SEQ ID NO: | (SEQ ID NO: |
| 651) | 658) | NO: 653) | NO: 654) | 655) | 656) |
| GYTFTSY | NPASGW | VSMYAPEPM | RASQGISRW | ASSLQS | QQAFSHPWT |
| (SEQ ID NO: | (SEQ ID NO: | DV (SEQ ID | LA (SEQ ID | (SEQ ID NO: | (SEQ ID NO: |
| 651) | 658) | NO: 657) | NO: 654) | 655) | 656) |
| GYTFTGHKM | SPYNGN | VSMYAPEPM | RASQGISRW | ASSLQS | QQAFSHPWT |
| H (SEQ ID | (SEQ ID NO: | DV (SEQ ID | LA (SEQ ID | (SEQ ID NO: | (SEQ ID NO: |
| NO 659) | 652) | NO: 653) | NO: 654) | 655) | 656) |
| GYTFTGHKM | SPYNGN | VSMYAPEPM | RASQGISRW | ASSLQS | QQAFSHPWT |
| H (SEQ ID | (SEQ ID NO: | DV (SEQ ID | LA (SEQ ID | (SEQ ID NO: | (SEQ ID NO: |
| NO 659) | 652) | NO: 657) | NO: 654) | 655) | 656) |
| GYTFTGHKM | NPASGW | VSMYAPEPM | RASQGISRW | ASSLQS | QQAFSHPWT |
| H (SEQ ID | (SEQ ID NO: | DV (SEQ ID | LA (SEQ ID | (SEQ ID NO: | (SEQ ID NO: |
| NO 659) | 658) | NO: 653) | NO: 654) | 655) | 656) |
| GYTFTGHKM | NPASGW | VSMYAPEPM | RASQGISRW | ASSLQS | QQAFSHPWT |
| H (SEQ ID | (SEQ ID NO: | DV (SEQ ID | LA (SEQ ID | (SEQ ID NO: | (SEQ ID NO: |
| NO 659) | 658) | NO: 657) | NO: 654) | 655) | 656) |
| GGSISSGSY | YYSGS (SEQ | GLGMYYHVP | RASQSVSSY | DASNRAT | QQYFHWPPT |
| (SEQ ID NO: | ID NO: 661) | FDI (SEQ ID | LA (SEQ ID | (SEQ ID NO: | (SEQ ID NO: |
| 660) | | NO: 662) | NO: 663) | 664) | 665) |
| GGSISSSSY | WIGGIYPSGS | DSGIGYSYAS | RASQSVGSN | GASTRAT | QQYFHFPLT |
| (SEQ ID NO: | TYY (SEQ ID | SHGYYYYMD | LA (SEQ ID | (SEQ ID NO: | (SEQ ID NO: |
| 622) | NO: 666) | V (SEQ ID | NO: 625) | 626) | 627) |
| | | NO: 624) | | | |
| GGSISSSSY | YYSGK (SEQ | DSGIGYSYAS | RASQSVGSN | GASTRAT | QQYFHFPLT |
| (SEQ ID NO: | ID NO: 667) | SHGYYYYMD | LA (SEQ ID | (SEQ ID NO: | (SEQ ID NO: |
| 622) | | V (SEQ ID | NO: 625) | 626) | 627) |
| | | NO: 624) | | | |
| GGSIGSGGY | WIGGIYPSGS | DSGIGYSYAS | RASQSVGSN | GASTRAT | QQYFHFPLT |
| (SEQ ID NO: | TYY (SEQ ID | SHGYYYYMD | LA (SEQ ID | (SEQ ID NO: | (SEQ ID NO: |
| 629) | NO: 666) | V (SEQ ID | NO: 625) | 626) | 627) |
| | | NO: 624) | | | |
| GGSIGSGGY | YYSGK (SEQ | DSGIGYSYAS | RASQSVGSN | GASTRAT | QQYFHFPLT |
| (SEQ ID NO: | ID NO: 667) | SHGYYYYMD | LA (SEQ ID | (SEQ ID NO: | (SEQ ID NO: |
| 629) | | V (SEQ ID | NO: 625) | 626) | 627) |
| | | NO: 624) | | | |
| GGSISSSSY | YYSGRT | DSGIGYSYAS | RASQSVGSN | GASTRAT | QQYFHFPLT |
| (SEQ ID NO: | (SEQ ID NO: | SHGYYYYMD | LA (SEQ ID | (SEQ ID NO: | (SEQ ID NO: |
| 622) | 668) | V (SEQ ID | NO: 625) | 626) | 627) |
| | | NO: 624) | | | |
| GGSIGSGGY | YYSGRT | DSGIGYSYAS | RASQSVGSN | GASTRAT | QQYFHFPLT |
| (SEQ ID NO: | (SEQ ID NO: | SHGYYYYMD | LA (SEQ ID | (SEQ ID NO: | (SEQ ID NO: |
| 629) | 668) | V (SEQ ID | NO: 625) | 626) | 627) |
| | | NO: 624) | | | |
| GGSIKSGGY | YYSGS (SEQ | DSGIGYSYAS | RASQSVGSN | GASTRAT | QQYFHFPLT |
| (SEQ ID NO: | ID NO: 623) | SHGYYYYMD | LA (SEQ ID | (SEQ ID NO: | (SEQ ID NO: |
| 669) | | V (SEQ ID | NO: 625) | 626) | 627) |
| | | NO: 624) | | | |
| GGSIKSGGY | YGSG (SEQ | DSGIGYSYAS | RASQSVGSN | GASTRAT | QQYFHFPLT |
| (SEQ ID NO: | ID NO: 628) | SHGYYYYMD | LA (SEQ ID | (SEQ ID NO: | (SEQ ID NO: |
| 669) | | V (SEQ ID | NO: 625) | 626) | 627) |
| | | NO: 624) | | | |
| GGSILSGGY | YYSGS (SEQ | DSGIGYSYAS | RASQSVGSN | GASTRAT | QQYFHFPLT |
| (SEQ ID NO: | ID NO: 623) | SHGYYYYMD | LA (SEQ ID | (SEQ ID NO: | (SEQ ID NO: |
| 670) | | V (SEQ ID | NO: 625) | 626) | 627) |
| | | NO: 624) | | | |
| GGSILSGGY | YGSG (SEQ | DSGIGYSYAS | RASQSVGSN | GASTRAT | QQYFHFPLT |
| (SEQ ID NO: | ID NO: 628) | SHGYYYYMD | LA (SEQ ID | (SEQ ID NO: | (SEQ ID NO: |
| 670) | | V (SEQ ID | NO: 625) | 626) | 627) |
| | | NO: 624) | | | |
| GGSIKSGGY | WIGGIYPSGS | DSGIGYSYAS | RASQSVGSN | GASTRAT | QQYFHFPLT |
| (SEQ ID NO: | TYY (SEQ ID | SHGYYYYMD | LA (SEQ ID | (SEQ ID NO: | (SEQ ID NO: |
| 669) | NO: 666) | V (SEQ ID | NO: 625) | 626) | 627) |
| | | NO: 624) | | | |
| GGSIKSGGY | YYSGK (SEQ | DSGIGYSYAS | RASQSVGSN | GASTRAT | QQYFHFPLT |
| (SEQ ID NO: | ID NO: 667) | SHGYYYYMD | LA (SEQ ID | (SEQ ID NO: | |
| 669) | | V (SEQ ID | NO: 625) | 626) | (SEQ ID NO: |
| | | NO: 624) | | | 627) |
| GGSILSGGY | WIGGIYPSGS | DSGIGYSYAS | RASQSVGSN | GASTRAT | QQYFHFPLT |
| (SEQ ID NO: | TYY (SEQ ID | SHGYYYYMD | LA (SEQ ID | (SEQ ID NO: | (SEQ ID NO: |
| 670) | NO: 666) | V (SEQ ID | NO: 625) | 626) | 627) |
| | | NO: 624) | | | |
| GGSILSGGY | YYSGK (SEQ | DSGIGYSYAS | RASQSVGSN | GASTRAT | QQYFHFPLT |
| (SEQ ID NO: | ID NO: 667) | SHGYYYYMD | LA (SEQ ID | (SEQ ID NO: | (SEQ ID NO: |
| 670) | | V (SEQ ID | NO: 625) | 626) | 627) |
| | | NO: 624) | | | |
| GGSIKSGGY | YYSGRT | DSGIGYSYAS | RASQSVGSN | GASTRAT | QQYFHFPLT |
| (SEQ ID NO: | (SEQ ID NO: | SHGYYYYMD | LA (SEQ ID | (SEQ ID NO: | (SEQ ID NO: |
| 669) | 668) | V (SEQ ID | NO: 625) | 626) | 627) |
| | | NO: 624) | | | |
| GGSIKSGGY | YYSGRT | DSGIGYSYAS | RASQSVGSN | GASTRAT | QQYFHFPLT |
| (SEQ ID NO: | (SEQ ID NO: | SHGYYYYMD | LA (SEQ ID | (SEQ ID NO: | (SEQ ID NO: |
| 669) | 668) | V (SEQ ID | NO: 625) | 626) | 627) |
| | | NO: 624) | | | |
| GGSISSGGY | YYSGS (SEQ | DSGIGYSYAS | RASQSVGSN | GASTRAT | QQYFHFPLT |
| (SEQ ID NO: | ID NO: 623) | SHGYYYYMD | LA (SEQ ID | (SEQ ID NO: | (SEQ ID NO: |
| 671) | | V (SEQ ID | NO: 625) | 626) | 627) |
| | | NO: 624) | | | |
| GGSISSGGY | YGSG (SEQ | DSGIGYSYAS | RASQSVGSN | GASTRAT | QQYFHFPLT |
| (SEQ ID NO: | ID NO: 628) | SHGYYYYMD | LA (SEQ ID | (SEQ ID NO: | (SEQ ID NO: |
| 671) | | V (SEQ ID | NO: 625) | 626) | 627) |
| | | NO: 624) | | | |
| GGSIESGGY | YYSGS (SEQ | DSGIGYSYAS | RASQSVGSN | GASTRAT | QQYFHFPLT |
| (SEQ ID NO: | ID NO: 623) | SHGYYYYMD | LA (SEQ ID | (SEQ ID NO: | (SEQ ID NO: |
| 672) | | V (SEQ ID | NO: 625) | 626) | 627) |
| | | NO: 624) | | | |
| GGSIESGGY | YGSG (SEQ | DSGIGYSYAS | RASQSVGSN | GASTRAT | QQYFHFPLT |
| (SEQ ID NO: | ID NO: 628) | SHGYYYYMD | LA (SEQ ID | (SEQ ID NO: | (SEQ ID NO: |
| 672) | | V (SEQ ID | NO: 625) | 626) | 627) |
| | | NO: 624) | | | |
| GGSISSGGY | WIGGIYPSGS | DSGIGYSYAS | RASQSVGSN | GASTRAT | QQYFHFPLT |
| (SEQ ID NO: | TYY (SEQ ID | SHGYYYYMD | LA (SEQ ID | (SEQ ID NO: | (SEQ ID NO: |
| 671) | NO: 666) | V (SEQ ID | NO: 625) | 626) | 627) |
| | | NO: 624) | | | |
| GGSISSGGY | YYSGK (SEQ | DSGIGYSYAS | RASQSVGSN | GASTRAT | QQYFHFPLT |
| (SEQ ID NO: | ID NO: 667) | SHGYYYYMD | LA (SEQ ID | (SEQ ID NO: | (SEQ ID NO: |
| 671) | | V (SEQ ID | NO: 625) | 626) | 627) |
| | | NO: 624) | | | |
| GGSIESGGY | WIGGIYPSGS | DSGIGYSYAS | RASQSVGSN | GASTRAT | QQYFHFPLT |
| (SEQ ID NO: | TYY (SEQ ID | SHGYYYYMD | LA (SEQ ID | (SEQ ID NO: | (SEQ ID NO: |
| 672) | NO: 666) | V (SEQ ID | NO: 625) | 626) | 627) |
| | | NO: 624) | | | |
| GGSIESGGY | YYSGK (SEQ | DSGIGYSYAS | RASQSVGSN | GASTRAT | QQYFHFPLT |
| (SEQ ID NO: | ID NO: 667) | SHGYYYYMD | LA (SEQ ID | (SEQ ID NO: | (SEQ ID NO: |
| 672) | | V (SEQ ID | NO: 625) | 626) | 627) |
| | | NO: 624) | | | |
| GGSISSGGY | YYSGRT | DSGIGYSYAS | RASQSVGSN | GASTRAT | QQYFHFPLT |
| (SEQ ID NO: | (SEQ ID NO: | SHGYYYYMD | LA (SEQ ID | (SEQ ID NO: | (SEQ ID NO: |
| 671) | 668) | V (SEQ ID | NO: 625) | 626) | 627) |
| | | NO: 624) | | | |
| GGSIESGGY | YYSGRT | DSGIGYSYAS | RASQSVGSN | GASTRAT | QQYFHFPLT |
| (SEQ ID NO: | (SEQ ID NO: | SHGYYYYMD | LA (SEQ ID | (SEQ ID NO: | (SEQ ID NO: |
| 672) | 668) | V (SEQ ID | NO: 625) | 626) | 627) |
| | | NO: 624) | | | |
| GYTFTSY | SPYNGN | VSMYAPEPM | RASQGISRW | ASSLQS | QQAFSHPWT |
| (SEQ ID NO: | (SEQ ID NO: | DV (SEQ ID | LA (SEQ ID | (SEQ ID NO: | (SEQ ID NO: |
| 651) | 652) | NO: 673) | NO: 654) | 655) | 656) |
| GYTFTSY | SPYNGN | VSMYAPEPM | RASQGISRW | ASSLQS | QQAFSHPWT |
| (SEQ ID NO: | (SEQ ID NO: | DV (SEQ ID | LA (SEQ ID | (SEQ ID NO: | (SEQ ID NO: |
| 651) | 652) | NO: 674) | NO: 654) | 655) | 656) |
| GYTFTSY | NPASGW | VSMYAPEPM | RASQGISRW | ASSLQS | QQAFSHPWT |
| (SEQ ID NO: | (SEQ ID NO: | DV (SEQ ID | LA (SEQ ID | (SEQ ID NO: | (SEQ ID NO: |
| 651) | 658) | NO: 673) | NO: 654) | 655) | 656) |
| GYTFTSY | NPASGW | VSMYAPEPM | RASQGISRW | ASSLQS | QQAFSHPWT |
| (SEQ ID NO: | (SEQ ID NO: | DV (SEQ ID | LA (SEQ ID | (SEQ ID NO: | (SEQ ID NO: |
| 651) | 658) | NO: 674) | NO: 654) | 655) | 656) |
| GYTFTGHKM | SPYNGN | VSMYAPEPM | RASQGISRW | ASSLQS | QQAFSHPWT |
| H (SEQ ID | (SEQ ID NO: | DV (SEQ ID | LA (SEQ ID | (SEQ ID NO: | (SEQ ID NO: |
| NO 659) | 652) | NO: 673) | NO: 654) | 655) | 656) |
| GYTFTGHKM | SPYNGN | VSMYAPEPM | RASQGISRW | ASSLQS | QQAFSHPWT |
| H (SEQ ID | (SEQ ID NO: | DV (SEQ ID | LA (SEQ ID | (SEQ ID NO: | (SEQ ID NO: |
| NO 659) | 652) | NO: 674) | NO: 654) | 655) | 656) |
| GYTFTGHKM | NPASGW | VSMYAPEPM | RASQGISRW | ASSLQS | QQAFSHPWT |
| H (SEQ ID | (SEQ ID NO: | DV (SEQ ID | LA (SEQ ID | (SEQ ID NO: | (SEQ ID NO: |
| NO: 659) | 658) | NO: 673) | NO: 654) | 655) | 656) |
| GYTFTGHKM | NPASGW | VSMYAPEPM | RASQGISRW | ASSLQS | QQAFSHPWT |
| H (SEQ ID | (SEQ ID NO: | DV (SEQ ID | LA (SEQ ID | (SEQ ID NO: | (SEQ ID NO: |
| NO: 659) | 658) | NO: 674) | NO: 654) | 655) | 656) |
| GYTFTSY | SPYNGN | VSRYAPEPM | RASQGISRW | ASSLQS | QQAFSHPWT |
| (SEQ ID NO: | (SEQ ID NO: | DV (SEQ ID | LA (SEQ ID | (SEQ ID NO: | (SEQ ID NO: |
| 651) | 652) | NO: 675) | NO: 654) | 655) | 656) |
| GYTFTSY | NPASGW | VSRYAPEPM | RASQGISRW | ASSLQS | QQAFSHPWT |
| (SEQ ID NO: | (SEQ ID NO: | DV (SEQ ID | LA (SEQ ID | (SEQ ID NO: | (SEQ ID NO: |
| 651) | 658) | NO: 675) | NO: 654) | 655) | 656) |
| GYTFTGHKM | SPYNGN | VSRYAPEPM | RASQGISRW | ASSLQS | QQAFSHPWT |
| H (SEQ ID | (SEQ ID NO: | DV (SEQ ID | LA (SEQ ID | (SEQ ID NO: | (SEQ ID NO: |
| NO: 659) | 652) | NO: 675) | NO: 654) | 655) | 656) |
| GYTFTGHKM | NPASGW | VSRYAPEPM | RASQGISRW | ASSLQS | QQAFSHPWT |
| H (SEQ ID | (SEQ ID NO: | DV (SEQ ID | LA (SEQ ID | (SEQ ID NO: | (SEQ ID NO: |
| NO: 659) | 658) | NO: 675) | NO: 654) | 655) | 656) |
| GYTFTSY | RPSVGS | VSMYAPEPM | RASQGISRW | ASSLQS | QQAFSHPWT |
| (SEQ ID NO: | (SEQ ID NO: | DV (SEQ ID | LA (SEQ ID | (SEQ ID NO: | (SEQ ID NO: |
| 651) | 676) | NO: 653) | NO: 654) | 655) | 656) |
| GYTFTSY | RPSVGS | VSMYAPEPM | RASQGISRW | ASSLQS | QQAFSHPWT |
| (SEQ ID NO: | (SEQ ID NO: | DV (SEQ ID | LA (SEQ ID | (SEQ ID NO: | (SEQ ID NO: |
| 651) | 676) | NO: 657) | NO: 654) | 655) | 656) |
| GYTFTSY | VPSGGS | VSMYAPEPM | RASQGISRW | ASSLQS | QQAFSHPWT |
| (SEQ ID NO: | (SEQ ID NO: | DV (SEQ ID | LA (SEQ ID | (SEQ ID NO: | (SEQ ID NO: |
| 651 | 677) | NO: 653) | NO: 654) | 655) | 656) |
| GYTFTSY | VPSGGS | VSMYAPEPM | RASQGISRW | ASSLQS | QQAFSHPWT |
| (SEQ ID NO: | (SEQ ID NO: | DV (SEQ ID | LA (SEQ ID | (SEQ ID NO: | (SEQ ID NO: |
| 651) | 677) | NO: 657) | NO: 654) | 655) | 656) |
| GYTFTGHKM | RPSVGS | VSMYAPEPM | RASQGISRW | ASSLQS | QQAFSHPWT |
| H (SEQ ID | (SEQ ID NO: | DV (SEQ ID | LA (SEQ ID | (SEQ ID NO: | (SEQ ID NO: |
| NO: 659) | 676) | NO: 653) | NO: 654) | 655) | 656) |
| GYTFTGHKM | RPSVGS | VSMYAPEPM | RASQGISRW | ASSLQS | QQAFSHPWT |
| H (SEQ ID | (SEQ ID NO: | DV (SEQ ID | LA (SEQ ID | (SEQ ID NO: | (SEQ ID NO: |
| NO: 659) | 676) | NO: 657) | NO: 654) | 655) | 656) |
| GYTFTGHKM | VPSGGS | VSMYAPEPM | RASQGISRW | ASSLQS | QQAFSHPWT |
| H (SEQ ID | (SEQ ID NO: | DV (SEQ ID | LA (SEQ ID | (SEQ ID NO: | (SEQ ID NO: |
| NO: 659) | 677) | NO: 653) | NO: 654) | 655) | 656) |
| GYTFTGHKM | VPSGGS | VSMYAPEPM | RASQGISRW | ASSLQS | QQAFSHPWT |
| H (SEQ ID | (SEQ ID NO: | DV (SEQ ID | LA (SEQ ID | (SEQ ID NO: | (SEQ ID NO: |
| NO: 659) | 677) | NO: 657) | NO: 654) | 655) | 656) |
| GYTFTSY | RPSVGS | VSMYAPEPM | RASQGISRW | ASSLQS | QQAFSHPWT |
| (SEQ ID NO: | (SEQ ID NO: | DV (SEQ ID | LA (SEQ ID | (SEQ ID NO: | (SEQ ID NO: |
| 651) | 676) | NO: 673) | NO: 654) | 655) | 656) |
| GYTFTSY | RPSVGS | VSMYAPEPM | RASQGISRW | ASSLQS | QQAFSHPWT |
| (SEQ ID NO: | (SEQ ID NO: | DV (SEQ ID | LA (SEQ ID | (SEQ ID NO: | (SEQ ID NO: |
| 651) | 676) | NO: 674) | NO: 654) | 655) | 656) |
| GYTFTSY | VPSGGS | VSMYAPEPM | RASQGISRW | ASSLQS | QQAFSHPWT |
| (SEQ ID NO: | (SEQ ID NO: | DV (SEQ ID | LA (SEQ ID | (SEQ ID NO: | (SEQ ID NO: |
| 651 | 677) | NO: 673) | NO: 654) | 655) | 656) |
| GYTFTSY | VPSGGS | VSMYAPEPM | RASQGISRW | ASSLQS | QQAFSHPWT |
| (SEQ ID NO: | (SEQ ID NO: | DV (SEQ ID | LA (SEQ ID | (SEQ ID NO: | (SEQ ID NO: |
| 651) | 677) | NO: 674) | NO: 654) | 655) | 656) |
| GYTFTGHKM | RPSVGS | VSMYAPEPM | RASQGISRW | ASSLQS | QQAFSHPWT |
| H (SEQ ID | (SEQ ID NO: | DV (SEQ ID | LA (SEQ ID | (SEQ ID NO: | (SEQ ID NO: |
| NO 659) | 676) | NO: 673) | NO: 654) | 655) | 656) |
| GYTFTGHKM | RPSVGS | VSMYAPEPM | RASQGISRW | ASSLQS | QQAFSHPWT |
| H (SEQ ID | (SEQ ID NO: | DV (SEQ ID | LA (SEQ ID | (SEQ ID NO: | (SEQ ID NO: |
| NO 659) | 676) | NO: 674) | NO: 654) | 655) | 656) |
| GYTFTGHKM | VPSGGS | VSMYAPEPM | RASQGISRW | ASSLQS | QQAFSHPWT |
| H (SEQ ID | (SEQ ID NO: | DV (SEQ ID | LA (SEQ ID | (SEQ ID NO: | (SEQ ID NO: |
| NO 659) | 677) | NO: 673) | NO: 654) | 655) | 656) |
| GYTFTGHKM | VPSGGS | VSMYAPEPM | RASQGISRW | ASSLQS | QQAFSHPWT |
| H (SEQ ID | (SEQ ID NO: | DV (SEQ ID | LA (SEQ ID | (SEQ ID NO: | (SEQ ID NO: |
| NO 659) | 677) | NO: 674) | NO: 654) | 655) | 656) |
| GYTFTSY | RPSVGS | VSRYAPEPM | RASQGISRW | ASSLQS | QQAFSHPWT |
| (SEQ ID NO: | (SEQ ID NO: | DV (SEQ ID | LA (SEQ ID | (SEQ ID NO: | (SEQ ID NO: |
| 651) | 676) | NO: 675) | NO: 654) | 655) | 656) |
| GYTFTSY | VPSGGS | VSRYAPEPM | RASQGISRW | ASSLQS | QQAFSHPWT |
| (SEQ ID NO: | (SEQ ID NO: | DV (SEQ ID | LA (SEQ ID | (SEQ ID NO: | (SEQ ID NO: |
| 651) | 677) | NO: 675) | NO: 654) | 655) | 656) |
| GYTFTGHKM | RPSVGS | VSRYAPEPM | RASQGISRW | ASSLQS | QQAFSHPWT |
| H (SEQ ID | (SEQ ID NO: | DV (SEQ ID | LA (SEQ ID | (SEQ ID NO: | (SEQ ID NO: |
| NO 659) | 676) | NO: 675) | NO: 654) | 655) | 656) |
| GYTFTGHKM | VPSGGS | VSRYAPEPM | RASQGISRW | ASSLQS | QQAFSHPWT |
| H (SEQ ID | (SEQ ID NO: | DV (SEQ ID | LA (SEQ ID | (SEQ ID NO: | (SEQ ID NO: |
| NO 659) | 677) | NO: 675) | NO: 654) | 655) | 656) |
| GYTFTSY | VPSGGS | VSMYAPEPM | RASQGISRW | ASSLQS | QQAFSHPWT |
| (SEQ ID NO: | (SEQ ID NO: | DV (SEQ ID | LA (SEQ ID | (SEQ ID NO: | (SEQ ID NO: |
| 651) | 678) | NO: 653) | NO: 654) | 655) | 656) |
| GYTFTSY | VPSGGS | VSMYAPEPM | RASQGISRW | ASSLQS | QQAFSHPWT |
| (SEQ ID NO: | (SEQ ID NO: | DV (SEQ ID | LA (SEQ ID | (SEQ ID NO: | (SEQ ID NO: |
| 651) | 678) | NO: 657) | NO: 654) | 655) | 656) |
| GYTFTGHKM | VPSGGS | VSMYAPEPM | RASQGISRW | ASSLQS | QQAFSHPWT |
| H (SEQ ID | (SEQ ID NO: | DV (SEQ ID | LA (SEQ ID | (SEQ ID NO: | (SEQ ID NO: |
| NO 659) | 678) | NO: 653) | NO: 654) | 655) | 656) |
| GYTFTGHKM | VPSGGS | VSMYAPEPM | RASQGISRW | ASSLQS | QQAFSHPWT |
| H (SEQ ID | (SEQ ID NO: | DV (SEQ ID | LA (SEQ ID | (SEQ ID NO: | (SEQ ID NO: |
| NO: 659) | 678) | NO: 657) | NO: 654) | 655) | 656) |
| GYTFTSY | VPSGGS | VSMYAPEPM | RASQGISRW | ASSLQS | QQAFSHPWT |
| (SEQ ID NO: | (SEQ ID NO: | DV (SEQ ID | LA (SEQ ID | (SEQ ID NO: | (SEQ ID NO: |
| 651) | 678) | NO: 673) | NO: 654) | 655) | 656) |
| GYTFTSY | VPSGGS | VSMYAPEPM | RASQGISRW | ASSLQS | QQAFSHPWT |
| (SEQ ID NO: | (SEQ ID NO: | DV (SEQ ID | LA (SEQ ID | (SEQ ID NO: | (SEQ ID NO: |
| 651) | 678) | NO: 674) | NO: 654) | 655) | 656) |
| GYTFTGHKM | VPSGGS | VSMYAPEPM | RASQGISRW | ASSLQS | QQAFSHPWT |
| H (SEQ ID | (SEQ ID NO: | DV (SEQ ID | LA (SEQ ID | (SEQ ID NO: | (SEQ ID NO: |
| NO: 659) | 678) | NO: 673) | NO: 654) | 655) | 656) |
| GYTFTGHKM | VPSGGS | VSMYAPEPM | RASQGISRW | ASSLQS | QQAFSHPWT |
| H (SEQ ID | (SEQ ID NO: | DV (SEQ ID | LA (SEQ ID | (SEQ ID NO: | (SEQ ID NO: |
| NO: 659) | 678) | NO: 674) | NO: 654) | 655) | 656) |
| GYTFTSY | VPSGGS | VSRYAPEPM | RASQGISRW | ASSLQS | QQAFSHPWT |
| (SEQ ID NO: | (SEQ ID NO: | DV (SEQ ID | LA (SEQ ID | (SEQ ID NO: | (SEQ ID NO: |
| 651) | 678) | NO: 675) | NO: 654) | 655) | 656) |
| GYTFTGHKM | VPSGGS | VSRYAPEPM | RASQGISRW | ASSLQS | QQAFSHPWT |
| H (SEQ ID | (SEQ ID NO: | DV (SEQ ID | LA (SEQ ID | (SEQ ID NO: | (SEQ ID NO: |
| NO: 659) | 678) | NO: 675) | NO: 654) | 655) | 656) |
| GYTFTSY | SPYNGN | VSMYAPEPM | RASQGISRW | ASSLQS | QQAFSHPWT |
| (SEQ ID NO: | (SEQ ID NO: | DV (SEQ ID | LA (SEQ ID | (SEQ ID NO: | (SEQ ID NO: |
| 679) | 652) | NO: 653) | NO: 654) | 655) | 656) |
| GYTFTSY | SPYNGN | VSMYAPEPM | RASQGISRW | ASSLQS | QQAFSHPWT |
| (SEQ ID NO: | (SEQ ID NO: | DV (SEQ ID | LA (SEQ ID | (SEQ ID NO: | (SEQ ID NO: |
| 679) | 652) | NO: 657) | NO: 654) | 655 | 656) |
| GYTFTSY | NPASGW | VSMYAPEPM | RASQGISRW | ASSLQS | QQAFSHPWT |
| (SEQ ID NO: | (SEQ ID NO: | DV (SEQ ID | LA (SEQ ID | (SEQ ID NO: | (SEQ ID NO: |
| 679) | 658) | NO: 653) | NO: 654) | 655) | 656) |
| GYTFTSY | NPASGW | VSMYAPEPM | RASQGISRW | ASSLQS | QQAFSHPWT |
| (SEQ ID NO: | (SEQ ID NO: | DV (SEQ ID | LA (SEQ ID | (SEQ ID NO: | (SEQ ID NO: |
| 679) | 658) | NO: 657) | NO: 654) | 655) | 656) |
| GYTFTSY | SPYNGN | VSMYAPEPM | RASQGISRW | ASSLQS | QQAFSHPWT |
| (SEQ ID NO: | (SEQ ID NO: | DV (SEQ ID | LA (SEQ ID | (SEQ ID NO: | (SEQ ID NO: |
| 680) | 652) | NO: 653) | NO: 654) | 655) | 656) |
| GYTFTSY | SPYNGN | VSMYAPEPM | RASQGISRW | ASSLQS | QQAFSHPWT |
| (SEQ ID NO: | (SEQ ID NO: | DV (SEQ ID | LA (SEQ ID | (SEQ ID NO: | (SEQ ID NO: |
| 680) | 652) | NO: 657) | NO: 654) | 655) | 656) |
| GYTFTSY | NPASGW | VSMYAPEPM | RASQGISRW | ASSLQS | QQAFSHPWT |
| (SEQ ID NO: | (SEQ ID NO: | DV (SEQ ID | LA (SEQ ID | (SEQ ID NO: | (SEQ ID NO: |
| 680) | 658) | NO: 653) | NO: 654) | 655) | 656) |
| GYTFTSY | NPASGW | VSMYAPEPM | RASQGISRW | ASSLQS | QQAFSHPWT |
| (SEQ ID NO: | (SEQ ID NO: | DV (SEQ ID | LA (SEQ ID | (SEQ ID NO: | (SEQ ID NO: |
| 680) | 658) | NO: 657) | NO: 654) | 655) | 656) |
| GYTFTSY | SPYNGN | VSMYAPEPM | RASQGISRW | ASSLQS | QQAFSHPWT |
| (SEQ ID NO: | (SEQ ID NO: | DV (SEQ ID | LA (SEQ ID | (SEQ ID NO: | (SEQ ID NO: |
| 679) | 652) | NO: 673) | NO: 654) | 655) | 656) |
| GYTFTSY | SPYNGN | VSMYAPEPM | RASQGISRW | ASSLQS | QQAFSHPWT |
| (SEQ ID NO: | (SEQ ID NO: | DV (SEQ ID | LA (SEQ ID | (SEQ ID NO: | (SEQ ID NO: |
| 679) | 652) | NO: 674) | NO: 654) | 655) | 656) |
| GYTFTSY | NPASGW | VSMYAPEPM | RASQGISRW | ASSLQS | QQAFSHPWT |
| (SEQ ID NO: | (SEQ ID NO: | DV (SEQ ID | LA (SEQ ID | (SEQ ID NO: | (SEQ ID NO: |
| 679) | 658) | NO: 673) | NO: 654) | 655) | 656) |
| GYTFTSY | NPASGW | VSMYAPEPM | RASQGISRW | ASSLQS | QQAFSHPWT |
| (SEQ ID NO: | (SEQ ID NO: | DV (SEQ ID | LA (SEQ ID | (SEQ ID NO: | (SEQ ID NO: |
| 679) | 658) | NO: 674) | NO: 654) | 655) | 656) |
| GYTFTSY | SPYNGN | VSMYAPEPM | RASQGISRW | ASSLQS | QQAFSHPWT |
| (SEQ ID NO: | (SEQ ID NO: | DV (SEQ ID | LA (SEQ ID | (SEQ ID NO: | (SEQ ID NO: |
| 680) | 652) | NO: 673) | NO: 654) | 655) | 656) |
| GYTFTSY | SPYNGN | VSMYAPEPM | RASQGISRW | ASSLQS | QQAFSHPWT |
| (SEQ ID NO: | (SEQ ID NO: | DV (SEQ ID | LA (SEQ ID | (SEQ ID NO: | (SEQ ID NO: |
| 680) | 652) | NO: 674) | NO: 654) | 655) | 656) |
| GYTFTSY | NPASGW | VSMYAPEPM | RASQGISRW | ASSLQS | QQAFSHPWT |
| (SEQ ID NO: | (SEQ ID NO: | DV (SEQ ID | LA (SEQ ID | (SEQ ID NO: | (SEQ ID NO: |
| 680) | 658) | NO: 673) | NO: 654) | 655) | 656) |
| GYTFTSY | NPASGW | VSMYAPEPM | RASQGISRW | ASSLQS | QQAFSHPWT |
| (SEQ ID NO: | (SEQ ID NO: | DV (SEQ ID | LA (SEQ ID | (SEQ ID NO: | (SEQ ID NO: |
| 680) | 658) | NO: 674) | NO: 654) | 655) | 656) |
| GYTFTSY | SPYNGN | VSRYAPEPM | RASQGISRW | ASSLQS | QQAFSHPWT |
| (SEQ ID NO: | (SEQ ID NO: | DV (SEQ ID | LA (SEQ ID | (SEQ ID NO: | (SEQ ID NO: |
| 679) | 652) | NO: 675) | NO: 654) | 655) | 656) |
| GYTFTSY | NPASGW | VSRYAPEPM | RASQGISRW | ASSLQS | QQAFSHPWT |
| (SEQ ID NO: | (SEQ ID NO: | DV (SEQ ID | LA (SEQ ID | (SEQ ID NO: | (SEQ ID NO: |
| 679) | 658) | NO: 675) | NO: 654) | 655) | 656) |
| GYTFTSY | SPYNGN | VSRYAPEPM | RASQGISRW | ASSLQS | QQAFSHPWT |
| (SEQ ID NO: | (SEQ ID NO: | DV (SEQ ID | LA (SEQ ID | (SEQ ID NO: | (SEQ ID NO: |
| 680) | 652) | NO: 675) | NO: 654) | 655) | 656) |
| GYTFTSY | NPASGW | VSRYAPEPM | RASQGISRW | ASSLQS | QQAFSHPWT |
| (SEQ ID NO: | (SEQ ID NO: | DV (SEQ ID | LA (SEQ ID | (SEQ ID NO: | (SEQ ID NO: |
| 680) | 658) | NO: 675) | NO: 654) | 655) | 656) |
| GYTFTSY | RPSVGS | VSMYAPEPM | RASQGISRW | ASSLQS | QQAFSHPWT |
| (SEQ ID NO: | (SEQ ID NO: | DV (SEQ ID | LA (SEQ ID | (SEQ ID NO: | (SEQ ID NO: |
| 679) | 676) | NO: 653) | NO: 654) | 655) | 656) |
| GYTFTSY | RPSVGS | VSMYAPEPM | RASQGISRW | ASSLQS | QQAFSHPWT |
| (SEQ ID NO: | (SEQ ID NO: | DV (SEQ ID | LA (SEQ ID | (SEQ ID NO: | (SEQ ID NO: |
| 679) | 676) | NO: 657) | NO: 654) | 655) | 656) |
| GYTFTSY | VPSGGS | VSMYAPEPM | RASQGISRW | ASSLQS | QQAFSHPWT |
| (SEQ ID NO: | (SEQ ID NO: | DV (SEQ ID | LA (SEQ ID | (SEQ ID NO: | (SEQ ID NO: |
| 679) | 677) | NO: 653) | NO: 654) | 655) | 656) |
| GYTFTSY | VPSGGS | VSMYAPEPM | RASQGISRW | ASSLQS | QQAFSHPWT |
| (SEQ ID NO: | (SEQ ID NO: | DV (SEQ ID | LA (SEQ ID | (SEQ ID NO: | (SEQ ID NO: |
| 679) | 677) | NO: 657) | NO: 654) | 655) | 656) |
| GYTFTSY | RPSVGS | VSMYAPEPM | RASQGISRW | ASSLQS | QQAFSHPWT |
| (SEQ ID NO: | (SEQ ID NO: | DV (SEQ ID | LA (SEQ ID | (SEQ ID NO: | (SEQ ID NO: |
| 680) | 676) | NO: 653) | NO: 654) | 655) | 656) |
| GYTFTSY | RPSVGS | VSMYAPEPM | RASQGISRW | ASSLQS | QQAFSHPWT |
| (SEQ ID NO: | (SEQ ID NO: | DV (SEQ ID | LA (SEQ ID | (SEQ ID NO: | (SEQ ID NO: |
| 680) | 676) | NO: 657) | NO: 654) | 655) | 656) |
| GYTFTSY | VPSGGS | VSMYAPEPM | RASQGISRW | ASSLQS | QQAFSHPWT |
| (SEQ ID NO: | (SEQ ID NO: | DV (SEQ ID | LA (SEQ ID | (SEQ ID NO: | (SEQ ID NO: |
| 680) | 677) | NO: 653) | NO: 654) | 655) | 656) |
| GYTFTSY | VPSGGS | VSMYAPEPM | RASQGISRW | ASSLQS | QQAFSHPWT |
| (SEQ ID NO: | (SEQ ID NO: | DV (SEQ ID | LA (SEQ ID | (SEQ ID NO: | (SEQ ID NO: |
| 680) | 677) | NO: 657) | NO: 654) | 655) | 656) |
| GYTFTSY | RPSVGS | VSMYAPEPM | RASQGISRW | ASSLQS | QQAFSHPWT |
| (SEQ ID NO: | (SEQ ID NO: | DV (SEQ ID | LA (SEQ ID | (SEQ ID NO: | (SEQ ID NO: |
| 679) | 676) | NO: 673) | NO: 654) | 655) | 656) |
| GYTFTSY | RPSVGS | VSMYAPEPM | RASQGISRW | ASSLQS | QQAFSHPWT |
| (SEQ ID NO: | (SEQ ID NO: | DV (SEQ ID | LA (SEQ ID | (SEQ ID NO: | (SEQ ID NO: |
| 679) | 676) | NO: 674) | NO: 654) | 655) | 656) |
| GYTFTSY | VPSGGS | VSMYAPEPM | RASQGISRW | ASSLQS | QQAFSHPWT |
| (SEQ ID NO: | (SEQ ID NO: | DV (SEQ ID | LA (SEQ ID | (SEQ ID NO: | (SEQ ID NO: |
| 679) | 677) | NO: 673) | NO: 654) | 655) | 656) |
| GYTFTSY | VPSGGS | VSMYAPEPM | RASQGISRW | ASSLQS | QQAFSHPWT |
| (SEQ ID NO: | (SEQ ID NO: | DV (SEQ ID | LA (SEQ ID | (SEQ ID NO: | (SEQ ID NO: |
| 679) | 677) | NO: 674) | NO: 654) | 655) | 656) |
| GYTFTSY | RPSVGS | VSMYAPEPM | RASQGISRW | ASSLQS | QQAFSHPWT |
| (SEQ ID NO: | (SEQ ID NO: | DV (SEQ ID | LA (SEQ ID | (SEQ ID NO: | (SEQ ID NO: |
| 680) | 676) | NO: 673) | NO: 654) | 655) | 656) |
| GYTFTSY | RPSVGS | VSMYAPEPM | RASQGISRW | ASSLQS | QQAFSHPWT |
| (SEQ ID NO: | (SEQ ID NO: | DV (SEQ ID | LA (SEQ ID | (SEQ ID NO: | (SEQ ID NO: |
| 680) | 676) | NO: 674) | NO: 654) | 655) | 656) |
| GYTFTSY | VPSGGS | VSMYAPEPM | RASQGISRW | ASSLQS | QQAFSHPWT |
| (SEQ ID NO: | (SEQ ID NO: | DV (SEQ ID | LA (SEQ ID | (SEQ ID NO: | (SEQ ID NO: |
| 680) | 677) | NO: 673) | NO: 654) | 655) | 656) |
| GYTFTSY | VPSGGS | VSMYAPEPM | RASQGISRW | ASSLQS | QQAFSHPWT |
| (SEQ ID NO: | (SEQ ID NO: | DV (SEQ ID | LA (SEQ ID | (SEQ ID NO: | (SEQ ID NO: |
| 680) | 677) | NO: 674) | NO: 654) | 655) | 656) |
| GYTFTSY | RPSVGS | VSRYAPEPM | RASQGISRW | ASSLQS | QQAFSHPWT |
| (SEQ ID NO: | (SEQ ID NO: | DV (SEQ ID | LA (SEQ ID | (SEQ ID NO: | (SEQ ID NO: |
| 679) | 676) | NO: 675) | NO: 654) | 655) | 656) |
| GYTFTSY | VPSGGS | VSRYAPEPM | RASQGISRW | ASSLQS | QQAFSHPWT |
| (SEQ ID NO: | (SEQ ID NO: | DV (SEQ ID | LA (SEQ ID | (SEQ ID NO: | (SEQ ID NO: |
| 679) | 677) | NO: 675) | NO: 654) | 655) | 656) |
| GYTFTSY | RPSVGS | VSRYAPEPM | RASQGISRW | ASSLQS | QQAFSHPWT |
| (SEQ ID NO: | (SEQ ID NO: | DV (SEQ ID | LA (SEQ ID | (SEQ ID NO: | (SEQ ID NO: |
| 680) | 676) | NO: 675) | NO: 654) | 655) | 656) |
| GYTFTSY | VPSGGS | VSRYAPEPM | RASQGISRW | ASSLQS | QQAFSHPWT |
| (SEQ ID NO: | (SEQ ID NO: | DV (SEQ ID | LA (SEQ ID | (SEQ ID NO: | (SEQ ID NO: |
| 680) | 677) | NO: 675) | NO: 654) | 655) | 656) |
| GYTFTSY | VPSGGS | VSMYAPEPM | RASQGISRW | ASSLQS | QQAFSHPWT |
| (SEQ ID NO: | (SEQ ID NO: | DV (SEQ ID | LA (SEQ ID | (SEQ ID NO: | (SEQ ID NO: |
| 679) | 678) | NO: 653) | NO: 654) | 655) | 656) |
| GYTFTSY | VPSGGS | VSMYAPEPM | RASQGISRW | ASSLQS | QQAFSHPWT |
| (SEQ ID NO: | (SEQ ID NO: | DV (SEQ ID | LA (SEQ ID | (SEQ ID NO: | (SEQ ID NO: |
| 679) | 678) | NO: 657) | NO: 654) | 655) | 656) |
| GYTFTSY | VPSGGS | VSMYAPEPM | RASQGISRW | ASSLQS | QQAFSHPWT |
| (SEQ ID NO: | (SEQ ID NO: | DV (SEQ ID | LA (SEQ ID | (SEQ ID NO: | (SEQ ID NO: |
| 680) | 678) | NO: 653) | NO: 654) | 655) | 656) |
| GYTFTSY | VPSGGS | VSMYAPEPM | RASQGISRW | ASSLQS | QQAFSHPWT |
| (SEQ ID NO: | (SEQ ID NO: | DV (SEQ ID | LA (SEQ ID | (SEQ ID NO: | (SEQ ID NO: |
| 680) | 678) | NO: 657) | NO: 654) | 655) | 656) |
| GYTFTSY | VPSGGS | VSMYAPEPM | RASQGISRW | ASSLQS | QQAFSHPWT |
| (SEQ ID NO: | (SEQ ID NO: | DV (SEQ ID | LA (SEQ ID | (SEQ ID NO: | (SEQ ID NO: |
| 679) | 678) | NO: 673) | NO: 654) | 655) | 656) |
| GYTFTSY | VPSGGS | VSMYAPEPM | RASQGISRW | ASSLQS | QQAFSHPWT |
| (SEQ ID NO: | (SEQ ID NO: | DV (SEQ ID | LA (SEQ ID | (SEQ ID NO: | (SEQ ID NO: |
| 679) | 678) | NO: 674) | NO: 654) | 655) | 656) |
| GYTFTSY | VPSGGS | VSMYAPEPM | RASQGISRW | ASSLQS | QQAFSHPWT |
| (SEQ ID NO: | (SEQ ID NO: | DV (SEQ ID | LA (SEQ ID | (SEQ ID NO: | (SEQ ID NO: |
| 680) | 678) | NO: 673) | NO: 654) | 655) | 656) |
| GYTFTSY | VPSGGS | VSMYAPEPM | RASQGISRW | ASSLQS | QQAFSHPWT |
| (SEQ ID NO: | (SEQ ID NO: | DV (SEQ ID | LA (SEQ ID | (SEQ ID NO: | (SEQ ID NO: |
| 680) | 678) | NO: 674) | NO: 654) | 655) | 656) |
| GYTFTSY | VPSGGS | VSRYAPEPM | RASQGISRW | ASSLQS | QQAFSHPWT |
| (SEQ ID NO: | (SEQ ID NO: | DV (SEQ ID | LA (SEQ ID | (SEQ ID NO: | (SEQ ID NO: |
| 679) | 678) | NO: 675) | NO: 654) | 655) | 656) |
| GYTFTSY | VPSGGS | VSRYAPEPM | RASQGISRW | ASSLQS | QQAFSHPWT |
| (SEQ ID NO: | (SEQ ID NO: | DV (SEQ ID | LA (SEQ ID | (SEQ ID NO: | (SEQ ID NO: |
| 680) | 678) | NO: 675) | NO: 654) | 655) | 656) |
| NYWMG (SEQ | YIRSSGGLTH | GLYSFDY | RASQGVNNF | AASTLQS | QQSYSTPRG |
| ID NO: 681) | YADSVKG | (SEQ ID NO: | LA (SEQ ID | (SEQ ID NO: | (SEQ ID NO: |
| | (SEQ ID NO: | 683) | NO: 684) | 685) | 686) |
| | 682) | | | | |
| GSISSSSYYW | SISYSGSTYY | ARDSLRYGM | KSSQSVLYSS | WASTRES | QQYALAPPR |
| G (SEQ ID | NPSLKS (SEQ | DV (SEQ ID | NNKNYLA | (SEQ ID NO: | T (SEQ ID NO: |
| NO: 699) | ID NO: 700) | NO: 701) | (SEQ ID NO: | 703) | 704) |
| | | | 702) | | |
| GSISSSSYYW | SISYSGSTYY | ARDSLRYGM | RASQSVSSN | GASTRAT | QVYNVWPRT |
| G (SEQ ID | NPSLKS (SEQ | DV (SEQ ID | LA (SEQ ID | (SEQ ID NO: | (SEQ ID NO: |
| NO: 699) | ID NO: 700) | NO: 701) | NO: 705) | 706) | 707) |
| SYGMT (SEQ | VISGSGGGTY | AKGPRVVGM | RASQGISSW | AASSLQS | QQVFSYPLT |
| ID NO: 708) | YADSVKG | DV (SEQ ID | LA (SEQ ID | (SEQ ID NO: | (SEQ ID NO: |
| | (SEQ ID NO: | NO: 710) | NO: 711) | 712) | 713) |
| | 709) | | | | |
| GSISSSSYYW | SISYSGSTYY | ARDSLRYGM | RASQSIGSW | KASSLES | QVYGSYSPR |
| G (SEQ ID | NPSLKS (SEQ | DV (SEQ ID | LA (SEQ ID | (SEQ ID NO: | T (SEQ ID NO: |
| NO: 699) | ID NO: 700) | NO: 701) | NO: 714) | 715) | 716) |
| GFTFSSY | AISGSGGSTS | ARLPQYSRP | RASQGISSW | AASSLQS | QQGHSFPLT |
| (SEQ ID NO: | YADSVKG | FDY (SEQ ID | LA (SEQ ID | (SEQ ID NO: | (SEQ ID NO: |
| 717) | (SEQ ID NO: | NO: 719) | NO: 720) | 721) | 722) |
| | 718) | | | | |
| SYRMH (SEQ | FIVPSGGSTS | VSRYAPEPM | RASQGISRW | AASSLQS | QQAFSHPWT |
| ID NO: 723) | YAQKFQG | DV (SEQ ID | LA (SEQ ID | (SEQ ID NO: | (SEQ ID NO: |
| | (SEQ ID NO: | NO: 725) | NO: 726) | 727) | 728) |
| | 724) | | | | |

In some embodiments, the ActRII antibody or an antigen-binding fragment thereof, has a heavy chain and light chain sequence having at least 90% sequence identity (e.g., 90%, 91%, 92%, 93%, 94%, 95%, 95%, 97%, 98%, 99% or 100% sequence identity) to a heavy chain and light chain sequence provided in Table 10. In some embodiments, the ActRII antibody or an antigen binding fragment thereof, has a heavy chain and light chain sequence from the same row of Table 10. In some embodiments, the heavy chain and light chain have the sequence of SEQ ID NOs: 508 and 509; 510 and 511; 557 and 558; 559 and 560; 561 and 562; 563 and 564; 565 and 566; 567 and 568; 569 and 570; 571 and 572; 573 and 574; or 575 and 576 (e.g., the heavy chain has at least 90% sequence identity (e.g., 90%, 91%, 92%, 93%, 94%, 95%, 95%, 97%, 98%, 99% or 100% sequence identity) to the sequence of the first SEQ ID NO: in each pair and the light chain has at least 90% sequence identity (e.g., 90%, 91%, 92%, 93%, 94%, 95%, 95%, 97%, 98%, 99% or 100% sequence identity) to the sequence of the second SEQ ID NO: in each pair).

TABLE 10
Exemplary heavy and light chain sequences of ActRII antibodies

| Heavy chain | Light chain |
|---|---|
| EVQLLESGGGLVQPGGSLRLSCAASGFTFRNS | QTVVTQEPSFSVSPGGTVTLTCGFNSGSVSTSY |
| AMSWVRQAPGKGLEWVSGISVTGGSTFYTDSV | WPSWYQQTPGQAPRTLIYNTNTRSSGVPDRFS |
| KGRFTVSRDNSRNTLYLQMNSLRAEDTAVYYC | GSILGNKAALTITGAQADDESDYYCVLWMGSGI |
| AKVYYYSFFDYWGQGTLVTVSSASTKGPSVFPL | WVFGGGTKLTVLGQPKANPTVTLFPPSSEELQA |
| APCSRSTSESTAALGCLVKDYFPEPVTVSWNS | NKATLVCLISDFYPGAVTVAWKADGSPVKAGVE |
| GALTSGVHTFPAVLQSSGLYSLSSVVTVPSSNF | TTKPSKQSNNKYAASSYLSLTPEQWKSHRSYS |
| GTQTYTCNVDHKPSNTKVDKTVERKCCVECPP | CQVTHEGSTVEKTVAPTECS (SEQ ID NO: 509) |
| CPAPPVAGPSVFLFPPKPKDTLMISRTPEVTCVV | |
| VDVSHEDPEVQFNWYVDGVEVHNAKTKPREEQ | |
| FNSTFRVVSVLTVVHQDWLNGKEYKCKVSNKG | |
| LPAPIEKTISKTKGQPREPQVYTLPPSREEMTKN | |
| QVSLTCLVKGFYPSDIAVEWESNGQPENNYKTT | |
| PPMLDSDGSFFLYSKLTVDKSRWQQGNVFSCS | |
| VMHEALHNHYTQKSLSLSPGK (SEQ ID NO: | |
| 508) | |
| EVQLLESGGGLVQPGGSLRLSCAASGFTFRND | QTVVTQEPSFSVSPGGTVTLTCGFNSGSVSTSY |
| AMSWVRQAPGKGLEWVSGISVTGGSTFYTDSV | YPSWYQQTPGQAPRTLIYNTNTRSSGVPDRFS |
| KGRFTVSRDNSRNTLYLQMNSLRAEDTAVYYC | GSILGNKAALTITGAQADDESDYYCVLYMGSGI |
| AKVYYTSFFDYWGQGTLVTVSSASTKGPSVFPL | WVFGGGTKLTVLGQPKANPTVTLFPPSSEELQA |
| APCSRSTSESTAALGCLVKDYFPEPVTVSWNS | NKATLVCLISDFYPGAVTVAWKADGSPVKAGVE |
| GALTSGVHTFPAVLQSSGLYSLSSVVTVPSSNF | TTKPSKQSNNKYAASSYLSLTPEQWKSHRSYS |
| GTQTYTCNVDHKPSNTKVDKTVERKCCVECPP | CQVTHEGSTVEKTVAPTECS (SEQ ID NO: 511) |
| CPAPPVAGPSVFLFPPKPKDTLMISRTPEVTCVV | |
| VDVSHEDPEVQFNWYVDGVEVHNAKTKPREEQ | |
| FNSTFRVVSVLTVVHQDWLNGKEYKCKVSNKG | |
| LPAPIEKTISKTKGQPREPQVYTLPPSREEMTKN | |
| QVSLTCLVKGFYPSDIAVEWESNGQPENNYKTT | |
| PPMLDSDGSFFLYSKLTVDKSRWQQGNVFSCS | |
| VMHEALHNHYTQKSLSLSPGK (SEQ ID NO: | |
| 510) | |
| QVQLVQSGAEVKKPGASVKVSCKASGYTFTSS | QSALTQPASVSGSPGQSITISCTGTSSDVGSYN |
| YINWVRQAPGQGLEWMGTINPVSGSTSYAQKF | YVNWYQQHPGKAPKLMIYGVSKRPSGVSNRFS |
| QGRVTMTRDTSISTAYMELSRLRSDDTAVYYCA | GSKSGNTASLTISGLQAEDEADYYCGTFAGGSY |
| RGGWFDYWGQGTLVTVSSASTKGPSVFPLAPS | YGVFGGGTKLTVLGQPKAAPSVTLFPPSSEELQ |
| SKSTSGGTAALGCLVKDYFPEPVTVSWNSGALT | ANKATLVCLISDFYPGAVTVAWKADSSPVKAGV |
| SGVHTFPAVLQSSGLYSLSSVVTVPSSSLGTQT | ETTTPSKQSNNKYAASSYLSLTPEQWKSHRSYS |
| YICNVNHKPSNTKVDKRVEPKSCDKTHTCPPCP | CQVTHEGSTVEKTVAPTECS (SEQ ID NO: 558) |
| APEAAGGPSVFLFPPKPKDTLMISRTPEVTCVV | |
| VDVSHEDPEVKFNWYVDGVEVHNAKTKPREEQ | |
| YNSTYRVVSVLTVLHQDWLNGKEYKCKVSNKA | |
| LPAPIEKTISKAKGQPREPQVYTLPPSREEMTKN | |
| QVSLTCLVKGFYPSDIAVEWESNGQPENNYKTT | |
| PPVLDSDGSFFLYSKLTVDKSRWQQGNVFSCS | |
| VMHEALHNHYTQKSLSLSPGK (SEQ ID NO: | |
| 557) | |
| QVQLVQSGAEVKKPGASVKVSCKASGYTFTSS | QSALTQPASVSGSPGQSITISCTGTSSDVGSYN |
| YINWVRQAPGQGLEWMGQINAASGMTRYAQK | YVNWYQQHPGKAPKLMIYGVSKRPSGVSNRFS |
| FQGRVTMTRDTSISTAYMELSRLRSDDTAVYYC | GSKSGNTASLTISGLQAEDEADYYCGTFAGGSY |
| ARGGWFDYWGQGTLVTVSSASTKGPSVFPLAP | YGVFGGGTKLTVLGQPKAAPSVTLFPPSSEELQ |
| SSKSTSGGTAALGCLVKDYFPEPVTVSWNSGA | ANKATLVCLISDFYPGAVTVAWKADSSPVKAGV |
| LTSGVHTFPAVLQSSGLYSLSSVVTVPSSSLGT | ETTTPSKQSNNKYAASSYLSLTPEQWKSHRSYS |
| QTYICNVNHKPSNTKVDKRVEPKSCDKTHTCPP | CQVTHEGSTVEKTVAPTECS (SEQ ID NO: 560) |
| CPAPEAAGGPSVFLFPPKPKDTLMISRTPEVTC | |
| VVVDVSHEDPEVKFNWYVDGVEVHNAKTKPRE | |
| EQYNSTYRVVSVLTVLHQDWLNGKEYKCKVSN | |
| KALPAPIEKTISKAKGQPREPQVYTLPPSREEMT | |
| KNQVSLTCLVKGFYPSDIAVEWESNGQPENNY | |
| KTTPPVLDSDGSFFLYSKLTVDKSRWQQGNV | |
| FSCSVMHEALHNHYTQKSLSLSPGK (SEQ ID | |
| NO: 559) | |
| QVQLVQSGAEVKKPGASVKVSCKASGYTFTSS | QSALTQPASVSGSPGQSITISCTGTSSDVGSYN |
| YINWVRQAPGQGLEWMGNINAAAGITLYAQKF | YVNWYQQHPGKAPKLMIYGVSKRPSGVSNRFS |
| QGRVTMTRDTSISTAYMELSRLRSDDTAVYYCA | GSKSGNTASLTISGLQAEDEADYYCGTFAGGSY |
| RGGWFDYWGQGTLVTVSSASTKGPSVFPLAPS | YGVFGGGTKLTVLGQPKAAPSVTLFPPSSEELQ |
| SKSTSGGTAALGCLVKDYFPEPVTVSWNSGALT | ANKATLVCLISDFYPGAVTVAWKADSSPVKAGV |
| SGVHTFPAVLQSSGLYSLSSVVTVPSSSLGTQT | ETTTPSKQSNNKYAASSYLSLTPEQWKSHRSYS |
| YICNVNHKPSNTKVDKRVEPKSCDKTHTCPPCP | CQVTHEGSTVEKTVAPTECS (SEQ ID NO: 562) |
| APEAAGGPSVFLFPPKPKDTLMISRTPEVTCVV | |
| VDVSHEDPEVKFNWYVDGVEVHNAKTKPREEQ | |
| YNSTYRVVSVLTVLHQDWLNGKEYKCKVSNKA | |
| LPAPIEKTISKAKGQPREPQVYTLPPSREEMTKN | |
| QVSLTCLVKGFYPSDIAVEWESNGQPENNYKTT | |
| PPVLDSDGSFFLYSKLTVDKSRWQQGNVFSCS | |
| VMHEALHNHYTQKSLSLSPGK (SEQ ID NO: | |
| 561) | |
| QVQLVQSGAEVKKPGASVKVSCKASGYTFTSS | QSALTQPASVSGSPGQSITISCTGTSSDVGSYN |
| YINWVRQAPGQGLEWMGGINPPAGTTSYAQKF | YVNWYQQHPGKAPKLMIYGVSKRPSGVSNRFS |
| QGRVTMTRDTSISTAYMELSRLRSDDTAVYYCA | GSKSGNTASLTISGLQAEDEADYYCGTFAGGSY |
| RGGWFDYWGQGTLVTVSSASTKGPSVFPLAPS | YGVFGGGTKLTVLGQPKAAPSVTLFPPSSEELQ |
| SKSTSGGTAALGCLVKDYFPEPVTVSWNSGALT | ANKATLVCLISDFYPGAVTVAWKADSSPVKAGV |
| SGVHTFPAVLQSSGLYSLSSVVTVPSSSLGTQT | ETTTPSKQSNNKYAASSYLSLTPEQWKSHRSYS |
| YICNVNHKPSNTKVDKRVEPKSCDKTHTCPPCP | CQVTHEGSTVEKTVAPTECS (SEQ ID NO: 564) |
| APEAAGGPSVFLFPPKPKDTLMISRTPEVTCVV | |
| VDVSHEDPEVKFNWYVDGVEVHNAKTKPREEQ | |
| YNSTYRVVSVLTVLHQDWLNGKEYKCKVSNKA | |
| LPAPIEKTISKAKGQPREPQVYTLPPSREEMTKN | |
| QVSLTCLVKGFYPSDIAVEWESNGQPENNYKTT | |
| PPVLDSDGSFFLYSKLTVDKSRWQQGNVFSCS | |
| VMHEALHNHYTQKSLSLSPGK (SEQ ID NO: | |
| 563) | |
| QVQLVQSGAEVKKPGASVKVSCKASGYTFTSS | |
| YINWVRQAPGQGLEWMGNINPATGHADYAQKF | |
| QGRVTMTRDTSISTAYMELSRLRSDDTAVYYCA | |
| RGGWFDYWGQGTLVTVSSASTKGPSVFPLAPS | |
| SKSTSGGTAALGCLVKDYFPEPVTVSWNSGALT | |
| SGVHTFPAVLQSSGLYSLSSVVTVPSSSLGTQT | QSALTQPASVSGSPGQSITISCTGTSSDVGSYN |
| YICNVNHKPSNTKVDKRVEPKSCDKTHTCPPCP | YVNWYQQHPGKAPKLMIYGVSKRPSGVSNRFS |
| APEAAGGPSVFLFPPKPKDTLMISRTPEVTCVV | GSKSGNTASLTISGLQAEDEADYYCGTFAGGSY |
| VDVSHEDPEVKFNWYVDGVEVHNAKTKPREEQ | YGVFGGGTKLTVLGQPKAAPSVTLFPPSSEELQ |
| YNSTYRVVSVLTVLHQDWLNGKEYKCKVSNKA | ANKATLVCLISDFYPGAVTVAWKADSSPVKAGV |
| LPAPIEKTISKAKGQPREPQVYTLPPSREEMTKN | ETTTPSKQSNNKYAASSYLSLTPEQWKSHRSYS |
| QVSLTCLVKGFYPSDIAVEWESNGQPENNYKTT | CQVTHEGSTVEKTVAPTECS (SEQ ID NO: 566) |
| PPVLDSDGSFFLYSKLTVDKSRWQQGNVFSCS | |
| VMHEALHNHYTQKSLSLSPGK (SEQ ID NO: | |
| 565) | |
| QVQLVQSGAEVKKPGASVKVSCKASGYTFTSS | QSALTQPASVSGSPGQSITISCTGTSSDVGSYN |
| YINWVRQAPGQGLEWMGTINPVSGSTSYAQKF | YVNWYQQHPGKAPKLMIYGVSKRPSGVSNRFS |
| QGRVTMTRDTSISTAYMELSRLRSDDTAVYYCA | GSKSGNTASLTISGLQAEDEADYYCGTFAGGSY |
| RGGWFDYWGQGTLVTVSSASTKGPSVFPLAPC | YGVFGGGTKLTVLGQPKAAPSVTLFPPSSEELQ |
| SRSTSESTAALGCLVKDYFPEPVTVSWNSGALT | ANKATLVCLISDFYPGAVTVAWKADSSPVKAGV |
| SGVHTFPAVLQSSGLYSLSSVVTVPSSNFGTQT | ETTTPSKQSNNKYAASSYLSLTPEQWKSHRSYS |
| YTCNVDHKPSNTKVDKTVERKCCVECPPCPAP | CQVTHEGSTVEKTVAPTECS (SEQ ID NO: 568) |
| PVAGPSVFLFPPKPKDTLMISRTPEVTCVVVDVS | |
| HEDPEVQFNWYVDGVEVHNAKTKPREEQFNST | |
| FRVVSVLTVVHQDWLNGKEYKCKVSNKGLPAPI | |
| EKTISKTKGQPREPQVYTLPPSREEMTKNQVSL | |
| TCLVKGFYPSDIAVEWESNGQPENNYKTTPPML | |
| DSDGSFFLYSKLTVDKSRWQQGNVFSCSVMHE | |
| ALHNHYTQKSLSLSPGK (SEQ ID NO: 567) | |
| QVQLVQSGAEVKKPGASVKVSCKASGYTFTSS | QSALTQPASVSGSPGQSITISCTGTSSDVGSYN |
| YINWVRQAPGQGLEWMGQINAASGMTRYAQK | YVNWYQQHPGKAPKLMIYGVSKRPSGVSNRFS |
| FQGRVTMTRDTSISTAYMELSRLRSDDTAVYYC | GSKSGNTASLTISGLQAEDEADYYCGTFAGGSY |
| ARGGWFDYWGQGTLVTVSSASTKGPSVFPLAP | YGVFGGGTKLTVLGQPKAAPSVTLFPPSSEELQ |
| CSRSTSESTAALGCLVKDYFPEPVTVSWNSGAL | ANKATLVCLISDFYPGAVTVAWKADSSPVKAGV |
| TSGVHTFPAVLQSSGLYSLSSVVTVPSSNFGTQ | ETTTPSKQSNNKYAASSYLSLTPEQWKSHRSYS |
| TYTCNVDHKPSNTKVDKTVERKCCVECPPCPA | CQVTHEGSTVEKTVAPTECS (SEQ ID NO: 570) |
| PPVAGPSVFLFPPKPKDTLMISRTPEVTCVVVDV | |
| SHEDPEVQFNWYVDGVEVHNAKTKPREEQFNS | |
| TFRVVSVLTVVHQDWLNGKEYKCKVSNKGLPA | |
| PIEKTISKTKGQPREPQVYTLPPSREEMTKNQV | |
| SLTCLVKGFYPSDIAVEWESNGQPENNYKTTPP | |
| MLDSDGSFFLYSKLTVDKSRWQQGNVFSCSVM | |
| HEALHNHYTQKSLSLSPGK (SEQ ID NO: 569) | |
| QVQLVQSGAEVKKPGASVKVSCKASGYTFTSS | QSALTQPASVSGSPGQSITISCTGTSSDVGSYN |
| YINWVRQAPGQGLEWMGNINAAAGITLYAQKF | YVNWYQQHPGKAPKLMIYGVSKRPSGVSNRFS |
| QGRVTMTRDTSISTAYMELSRLRSDDTAVYYCA | GSKSGNTASLTISGLQAEDEADYYCGTFAGGSY |
| RGGWFDYWGQGTLVTVSSASTKGPSVFPLAPC | YGVFGGGTKLTVLGQPKAAPSVTLFPPSSEELQ |
| SRSTSESTAALGCLVKDYFPEPVTVSWNSGALT | ANKATLVCLISDFYPGAVTVAWKADSSPVKAGV |
| SGVHTFPAVLQSSGLYSLSSVVTVPSSNFGTQT | ETTTPSKQSNNKYAASSYLSLTPEQWKSHRSYS |
| YTCNVDHKPSNTKVDKTVERKCCVECPPCPAP | CQVTHEGSTVEKTVAPTECS (SEQ ID NO: 572) |
| PVAGPSVFLFPPKPKDTLMISRTPEVTCVVVDVS | |
| HEDPEVQFNWYVDGVEVHNAKTKPREEQFNST | |
| FRVVSVLTVVHQDWLNGKEYKCKVSNKGLPAPI | |
| EKTISKTKGQPREPQVYTLPPSREEMTKNQVSL | |
| TCLVKGFYPSDIAVEWESNGQPENNYKTTPPML | |
| DSDGSFFLYSKLTVDKSRWQQGNVFSCSVMHE | |
| ALHNHYTQKSLSLSPGK (SEQ ID NO: 571) | |
| QVQLVQSGAEVKKPGASVKVSCKASGYTFTSS | QSALTQPASVSGSPGQSITISCTGTSSDVGSYN |
| YINWVRQAPGQGLEWMGGINPPAGTTSYAQKF | YVNWYQQHPGKAPKLMIYGVSKRPSGVSNRFS |
| QGRVTMTRDTSISTAYMELSRLRSDDTAVYYCA | GSKSGNTASLTISGLQAEDEADYYCGTFAGGSY |
| RGGWFDYWGQGTLVTVSSASTKGPSVFPLAPC | YGVFGGGTKLTVLGQPKAAPSVTLFPPSSEELQ |
| SRSTSESTAALGCLVKDYFPEPVTVSWNSGALT | ANKATLVCLISDFYPGAVTVAWKADSSPVKAGV |
| SGVHTFPAVLQSSGLYSLSSVVTVPSSNFGTQT | ETTTPSKQSNNKYAASSYLSLTPEQWKSHRSYS |
| YTCNVDHKPSNTKVDKTVERKCCVECPPCPAP | CQVTHEGSTVEKTVAPTECS (SEQ ID NO: 574) |
| PVAGPSVFLFPPKPKDTLMISRTPEVTCVVVDVS | |
| HEDPEVQFNWYVDGVEVHNAKTKPREEQFNST | |
| FRVVSVLTVVHQDWLNGKEYKCKVSNKGLPAPI | |
| EKTISKTKGQPREPQVYTLPPSREEMTKNQVSL | |
| TCLVKGFYPSDIAVEWESNGQPENNYKTTPPML | |
| DSDGSFFLYSKLTVDKSRWQQGNVFSCSVMHE | |
| ALHNHYTQKSLSLSPGK (SEQ ID NO: 573) | |
| QVQLVQSGAEVKKPGASVKVSCKASGYTFTSS | QSALTQPASVSGSPGQSITISCTGTSSDVGSYN |
| YINWVRQAPGQGLEWMGNINPATGHADYAQKF | YVNWYQQHPGKAPKLMIYGVSKRPSGVSNRFS |
| QGRVTMTRDTSISTAYMELSRLRSDDTAVYYCA | GSKSGNTASLTISGLQAEDEADYYCGTFAGGSY |
| RGGWFDYWGQGTLVTVSSASTKGPSVFPLAPC | YGVFGGGTKLTVLGQPKAAPSVTLFPPSSEELQ |
| SRSTSESTAALGCLVKDYFPEPVTVSWNSGALT | ANKATLVCLISDFYPGAVTVAWKADSSPVKAGV |
| SGVHTFPAVLQSSGLYSLSSVVTVPSSNFGTQT | ETTTPSKQSNNKYAASSYLSLTPEQWKSHRSYS |
| YTCNVDHKPSNTKVDKTVERKCCVECPPCPAP | CQVTHEGSTVEKTVAPTECS (SEQ ID NO: 576) |
| PVAGPSVFLFPPKPKDTLMISRTPEVTCVVVDVS | |
| HEDPEVQFNWYVDGVEVHNAKTKPREEQFNST | |
| FRVVSVLTVVHQDWLNGKEYKCKVSNKGLPAPI | |
| EKTISKTKGQPREPQVYTLPPSREEMTKNQVSL | |
| TCLVKGFYPSDIAVEWESNGQPENNYKTTPPML | |
| DSDGSFFLYSKLTVDKSRWQQGNVFSCSVMHE | |
| ALHNHYTQKSLSLSPGK (SEQ ID NO: 575) | |

In some embodiments, the ActRII signaling inhibitor is an ActRII ligand trap. ActRII ligand traps are polypeptides that contain an extracellular portion of ActRIIA and/or ActRIIB that are capable of binding to one or more ActRII ligands (e.g., activin A, activin B, myostatin, or GDF11). The extracellular portion of ActRIIA and/or ActRIIB may be fused to a moiety (e.g., an Fc domain, an Fc domain monomer, an albumin-binding peptide, a fibronectin domain, or a human serum albumin) by way of a linker. ActRII ligand traps can reduce or inhibit the binding of ActRII ligands to endogenous activin type II receptors, thereby reducing ActRII signaling. As the ActRII ligand traps contain the extracellular portion of the receptor, they will be soluble and able to bind to and sequester ligands (e.g., activins A and B, myostatin, GDF11) without activating intracellular signaling pathways.

In some embodiments, the ActRII ligand trap is an ActRIIA ligand trap. The ActRIIA ligand trap may contain an extracellular portion of wild-type ActRIIA (e.g., human or murine ActRIIA) or may contain an extracellular portion of wild-type ActRIIA that contains one or more amino acid substitutions relative to the wild-type human extracellular ActRIIA. The wild-type amino acid sequence of the extracellular portion of human ActRIIA is shown below.

Human ActRIIA, extracellular portion (SEQ ID NO:
73):
GAILGRSETQECLFFNANWEKDRTNQTGVEPCYGDKDKRRHCFATWKNIS
GSIEIVKQGCWLDDINCYDRTDCVEKKDSPEVYFCCCEGNMCNEKFSYFP
EMEVTQPTS

An ActRIIA ligand trap may contain the sequence of SEQ ID NO: 73 or a variant thereof that contains one or more amino acid substitutions. In some embodiments, the ActRIIA ligand trap contains a portion of SEQ ID NO: 73 (e.g., a contiguous portion that is shortened by the removal of amino acids from the N-terminus, C-terminus, or both) or a variant thereof that contains one or more amino acid substitutions. In some embodiments, the ActRIIA ligand trap contains the sequence of SEQ ID NO: 73 or a portion thereof with additional amino acids at the C-terminus from the wild-type sequence of ActRIIA (SEQ ID NO: 75). An exemplary sequence of a portion of wild-type ActRIIA protein that is shortened at the N-terminus and includes additional amino acids from SEQ ID NO: 75 at the C-terminus that can be included in an ActRIIA ligand trap is provided below:

(SEQ ID NO: 729)
ILGRSETQECLFFNANWEKDRTNQTGVEPCYGDKDKRRHCFATWKNISGS
IEIVKQGCWLDDINCYDRTDCVEKKDSPEVYFCCCEGNMCNEKFSYFPEM
EVTQPTSNPVTPKPP

Studies have shown that BMP9 binds ActRIIB with about 300-fold higher binding affinity than ActRIIA (see, e.g., Townson et al., J. Biol. Chem. 287:27313, 2012). ActRIIA-Fc is known to have a longer half-life compared to ActRIIB-Fc. Described herein below are ActRIIA ligand traps containing extracellular ActRIIA variants that are constructed by introducing amino acid residues of ActRIIB to ActRIIA, with the goal of imparting physiological properties conferred by ActRIIB, while also maintaining beneficial physiological and pharmacokinetic properties of ActRIIA. The optimum peptides promote hematopoiesis (e.g., increase red blood cell count, hemoglobin levels, hematocrit, reticulocytes, platelet levels (e.g., platelet count), and/or neutrophil levels (e.g., neutrophil count)), while retaining low binding-affinity to BMP9 and longer serum half-life as an Fc fusion protein, for example. The preferred ActRIIA variants also exhibit similar or improved binding to activins and/or myostatin compared to wild-type ActRIIA, which allows them to compete with endogenous activin receptors for ligand binding and reduce or inhibit endogenous activin receptor signaling. These variants can be used to treat a cytopenia (e.g., anemia, thrombocytopenia, and/or neutropenia) associated with a myelodysplastic syndrome or myelofibrosis by increasing hemoglobin levels, hematocrit, red blood cell count (e.g., increasing red blood cell production and/or red cell mass or volume), or erythroid progenitor maturation and/or differentiation (e.g., the maturation and/or differentiation of early-stage or late- (e.g., terminal) stage erythroid progenitors into proerythroblasts, reticulocytes, or red blood cells), reducing the accumulation of red blood cell progenitor cells (e.g., by stimulating progenitor cells to progress to maturation), increasing late-stage precursor (erythroid precursor) maturation (e.g., terminal maturation, such as the maturation of reticulocytes into red blood cells, or the maturation of erythroblasts into reticulocytes and/or red blood cells), recruiting early-stage progenitors into the erythroid lineage, increasing the number of early-stage erythroid precursors and/or progenitors, promoting the progression of erythroid precursors and/or progenitors through erythropoiesis (e.g., progression through the erythropoiesis pathway), increasing proerythroblasts, increasing reticulocytes, increasing platelet levels (e.g., increasing platelet count, megakaryocyte differentiation and/or maturation, megakaryocyte progenitor renewal, and/or platelet production), reducing the accumulation of platelet progenitor cells (e.g., by stimulating progenitor cells to progress to maturation), increasing neutrophil levels (e.g., increasing neutrophil count, e.g., increasing neutrophil production), and/or increasing the differentiation and/or maturation of progenitor cells (e.g., myeloid progenitors, myeloblasts, or myelocytes) into neutrophils. In some embodiments, amino acid substitutions may be introduced to an extracellular ActRIIA variant to reduce or remove the binding affinity of the variant to BMP9.

ActRIIA ligand traps described herein can include an extracellular ActRIIA variant having at least one amino acid substitution relative to the wild-type extracellular ActRIIA having the sequence of SEQ ID NO: 73. Possible amino acid substitutions at 27 different positions may be introduced to an extracellular ActRIIA variant (Table 11). In some embodiments, an extracellular ActRIIA variant may have at least 85% (e.g., at least 85%, 87%, 90%, 92%, 95%, 97%, or greater) amino acid sequence identity to the sequence of a wild-type extracellular ActRIIA (SEQ ID NO: 73). An extracellular ActRIIA variant may have one or more (e.g., 1-27, 1-25, 1-23, 1-21, 1-19, 1-17, 1-15, 1-13, 1-11, 1-9, 1-7, 1-5, 1-3, or 1-2; e.g., 1, 2, 3, 4, 5, 6, 7, 8, 9, 10, 11, 12, 13, 14, 15, 16, 17, 18, 19, 20, 21, 22, 23, 24, 25, 26, or 27) amino acid substitutions relative the sequence of a wild-type extracellular ActRIIA (SEQ ID NO: 73). In some embodiments, an extracellular ActRIIA variant (e.g., an extracellular ActRIIA variant having a sequence of SEQ ID NO: 1) may include amino acid substitutions at all of the 27 positions as listed in Table 11. In some embodiments, an extracellular ActRIIA variant may include amino acid substitutions at a number of positions, e.g., at 2, 4, 6, 8, 10, 12, 14, 16, 18, 20, 22, 24, or 26 out of the 27 positions, as listed in Table 11.

Amino acid substitutions can worsen or improve the activity and/or binding affinity of the ActRIIA variants of the invention. To maintain polypeptide function, it is important that the lysine (K) at position X₁₇ in the sequences shown in Tables 11 and 12 (SEQ ID NOs: 1-72 (e.g., SEQ ID NOs: 6-72)) be retained. Substitutions at that position can lead to a loss of activity. For example, an ActRIIA variant having the sequence: GAILGRSETQECLFYNANWELERTNQTGVERCEGEKDKRLHCYATWRNISGSIEIVAKGCWLDDFNCYD RTDCVETEENPQVYFCCCEGNMCNEKFSYFPEMEVTQPTS (SEQ ID NO: 85) has reduced activity in vivo, indicating that the substitution of alanine (A) for lysine (K) at X₁₇ is not tolerated. ActRIIA variants of the invention, including variants in Tables 11 and 12 (e.g., SEQ ID NOs: 1-72 (e.g., SEQ ID NOs: 6-72), therefore, retain amino acid K at position X₁₇.

The ActRIIA variants of the invention preferably have reduced, weak, or no substantial binding to BMP9. BMP9 binding is reduced in ActRIIA variants (e.g., reduced compared to wild-type ActRIIA) containing the amino acid sequence TEEN (SEQ ID NO: 76) at positions X₂₃, X₂₄, X₂₅, and X₂₆, as well as in variants that maintain the amino acid K at position X₂₄ and have the amino acid sequence TKEN (SEQ ID NO: 77) at positions X₂₃, X₂₄, X₂₅, and X₂₆. The sequences TEEN (SEQ ID NO: 76) and TKEN (SEQ ID NO: 77) can be employed interchangeably in the ActRIIA variants (e.g., the variants in Tables 11 and 12, e.g., SEQ ID NOs: 1-72 (e.g., SEQ ID NOs: 6-72)) of the invention to provide reduced BMP9 binding.

The ActRIIA variants of the invention may further include a C-terminal extension (e.g., additional amino acids at the C-terminus). The C-terminal extension can add one or more additional amino acids at the C-terminus (e.g., 1, 2, 3, 4, 5, 6 or more additional amino acids) to any of the variants shown in Tables 11 and 12 (e.g., SEQ ID NOs: 1-70 (e.g., SEQ ID NOs: 6-70)). The C-terminal extension may correspond to sequence from the same position in wild-type ActRIIA. One potential C-terminal extension that can be included in the ActRIIA variants of the invention is amino acid sequence NP. For example, a sequence including the C-terminal extension NP is SEQ ID NO: 71 (e.g., SEQ ID NO: 69 with a C-terminal extension of NP). Another exemplary C-terminal extension that can be included in the ActRIIA variants of the invention is amino acid sequence NPVTPK (SEQ ID NO: 78). For example, a sequence including the C-terminal extension NPVTPK (SEQ ID NO: 78) is SEQ ID NO: 72 (e.g., SEQ ID NO: 69 with a C-terminal extension of NPVTPK (SEQ ID NO: 78)).

TABLE 11
Amino acid substitutions in an extracellular ActRIIA variant having
a sequence of any one of SEQ ID NOs: 1-5

GAILGRSETQECLX₁X₂NANWX₃X₄X₅X₆TNQTGVEX₇CX₈GX₉X₁₀X₁₁X₁₂X₁₃X₁₄HCX₁₅ATWX₁₆NISGSIEIV
X₁₇X₁₈GCX₁₉X₂₀X₂₁DX₂₂NCYDRTDCVEX₂₃X₂₄X₂₅X₂₆PX₂₇VYFCCCEGNMCNEKFSYFPEMEVTQPTS (SEQ ID NO:
1)
GAILGRSETQECLFX₂NANWX₃X₄X₅X₆TNQTGVEX₇CX₈GX₉KX₁₁X₁₂X₁₃X₁₄HCX₁₅ATWX₁₆NISGSIEIVX₁₇X₁₈GCX₁₉
X₂₀X₂₁DX₂₂NCYDRTDCVEX₂₃X₂₄X₂₅X₂₆PX₂₇VYFCCCEGNMCNEKFSYFPEMEVTQPTS (SEQ ID NO: 2)
GAILGRSETQECLFX₂NANWEX₄X₅RTNQTGVEX₇CX₈GX₉KDKRX₁₄HCX₁₅ATWX₁₆NISGSIEIVKX₁₈GCWLDDX₂₂N
CYDRTDCVEX₂₃X₂₄X₂₅X₂₆PX₂₇VYFCCCEGNMCNEKFSYFPEMEVTQPTS (SEQ ID NO: 3)
GAILGRSETQECLFX₂NANWEX₄DRTNQTGVEX₇CX₈GX₉KDKRX₁₄HCX₁₅ATWX₁₆NISGSIEIVKX₁₈GCWLDDX₂₂N
CYDRTDCVEX₂₃KX₂₅X₂₆PX₂₇VYFCCCEGNMCNEKFSYFPEMEVTQPTS (SEQ ID NO: 4)
GAILGRSETQECLFX₂NANWEX₄DRTNQTGVEPCX₈GX₉KDKRX₁₄HCFATWKNISGSIEIVKX₁₈GCWLDDINCYDRT
DCVEX₂₃KX₂₅X₂₆PX₂₇VYFCCCEGNMCNEKFSYFPEMEVTQPTS (SEQ ID NO: 5)

| | | | |
|---|---|---|---|
| X₁ | F or Y | X₁₅ | F or Y |
| X₂ | F or Y | X₁₆ | K, R, or A |

GAILGRSETQECLX₁X₂NANWX₃X₄X₅X₆TNQTGVEX₇CX₈GX₉X₁₀X₁₁X₁₂X₁₃X₁₄HCX₁₅ATWX₁₆NISGSIEIV
X₁₇X₁₈GCX₁₉X₂₀X₂₁DX₂₂NCYDRTDCVEX₂₃X₂₄X₂₅X₂₆PX₂₇VYFCCCEGNMCNEKFSYFPEMEVTQPTS (SEQ ID NO:
1)
GAILGRSETQECLFX₂NANWX₃X₄X₅X₆TNQTGVEX₇CX₈GX₉KX₁₁X₁₂X₁₃X₁₄HCX₁₅ATWX₁₆NISGSIEIVX₁₇X₁₈GCX₁₉
X₂₀X₂₁DX₂₂NCYDRTDCVEX₂₃X₂₄X₂₅X₂₆PX₂₇VYFCCCEGNMCNEKFSYFPEMEVTQPTS (SEQ ID NO: 2)
GAILGRSETQECLFX₂NANWEX₄X₅RTNQTGVEX₇CX₈GX₉KDKRX₁₄HCX₁₅ATWX₁₆NISGSIEIVKX₁₈GCWLDDX₂₂N
CYDRTDCVEX₂₃X₂₄X₂₅X₂₆PX₂₇VYFCCCEGNMCNEKFSYFPEMEVTQPTS (SEQ ID NO: 3)
GAILGRSETQECLFX₂NANWEX₄DRTNQTGVEX₇CX₈GX₉KDKRX₁₄HCX₁₅ATWX₁₆NISGSIEIVKX₁₈GCWLDDX₂₂N
CYDRTDCVEX₂₃KX₂₅X₂₆PX₂₇VYFCCCEGNMCNEKFSYFPEMEVTQPTS (SEQ ID NO: 4)
GAILGRSETQECLFX₂NANWEX₄DRTNQTGVEPCX₈GX₉KDKRX₁₄HCFATWKNISGSIEIVKX₁₈GCWLDDINCYDRT
DCVEX₂₃KX₂₅X₂₆PX₂₇VYFCCCEGNMCNEKFSYFPEMEVTQPTS (SEQ ID NO: 5)

| | | | |
|---|---|---|---|
| X₃ | E or A | X₁₇ | |
| X₄ | K or L | X₁₈ | Q or K |
| X₅ | D or E | X₁₉ | W or A |
| X₆ | R or A | X₂₀ | L or A |
| X₇ | P or R | X₂₁ | D, K, R, A, F, G, M, N, or I |
| X₈ | Y or E | X₂₂ | I, F, or A |
| X₉ | D or E | X₂₃ | K or T |
| X₁₀ | K or Q | X₂₄ | K or E |
| X₁₁ | D or A | X₂₅ | D or E |
| X₁₂ | K or A | X₂₆ | S or N |
| X₁₃ | R or A | X₂₇ | E or Q |
| X₁₄ | R or L | | |

In some embodiments of the extracellular ActRIIA variant having the sequence of SEQ ID NO: 1 or 2, X₃ is E, X₆ is R, X₁₁ is D, X₁₂ is K, X₁₃ is R, X₁₆ is K or R, X₁₇ is K, X₁₉ is W, X₂₀ is L, X₂₁ is D, and X₂₂ is I or F. In some embodiments of the extracellular ActRIIA variant having the sequence of SEQ ID NO: 1, X₂ is Y; X₄ is L; X₈ is E; X₉ is E; X₁₄ is L; X₁₈ is K; X₂₃ is T; X₂₅ is E; X₂₆ is N; and X₂₇ is Q. These substitutions in SEQ ID NO: 1 can also be made in SEQ ID NOs: 2-5. In some embodiments of the extracellular ActRIIA variant having the sequence of SEQ ID NO: 1, X₁ is F or Y; X₂ is Y; X₄ is L; X₅ is D or E; X₇ is P or R; X₈ is E; X₉ is E; X₁₀ is K or Q; X₁₄ is L; X₁₅ is F or Y; X₁₆ is K or R; X₁₈ is K; X₂₂ is I or F; X₂₃ is T; X₂₄ is K or E; X₂₅ is E; X₂₆ is N; and X₂₇ is Q. In some embodiments of the extracellular ActRIIA variant having the sequence of SEQ ID NO: 1, X₁ is F or Y; X₂ is Y; X₃ is E; X₄ is L; X₅ is D or E; X₆ is R; X₇ is P or R; X₈ is E; X₉ is E; X₁₀ is K or Q; X₁₁ is D; X₁₂ is K; X₁₃ is R; X₁₄ is L; X₁₅ is F or Y; X₁₆ is K or R; X₁₇ is K; X₁₈ is K; X₁₉ is W; X₂₀ is L; X₂₁ is D; X₂₂ is I or F; X₂₃ is T; X₂₄ is K or E; X₂₅ is E; X₂₆ is N; and X₂₇ is Q. In some embodiments of the extracellular ActRIIA variant having the sequence of SEQ ID NO: 1 or 2, X₁₇ is K. In some embodiments of the extracellular ActRIIA variant having the sequence of SEQ ID NOs: 1-3, X₁₇ is K, X₂₃ is T, X₂₄ is E, X₂₅ is E, and X₂₆ is N. In some embodiments of the extracellular ActRIIA variant having the sequence of any one of SEQ ID NOs: 1-5, X₁₇ is K, X₂₃ is T, X₂₄ is K, X₂₅ is E, and X₂₆ is N.

In some embodiments, an ActRIIA ligand trap described herein includes an extracellular ActRIIA variant having a sequence of any one of SEQ ID NOs: 6-72 (Table 12).

TABLE 12
Extracellular ActRIIA variants having the sequences of SEQ ID NOs: 6-72

| SEQ ID NO | Amino Acid Sequence |
|---|---|
| 6 | GAILGRSETQECLFYNANWELDRTNQTGVEPCEGEKDKRLHCFATWKNISGSIEIV |
| | KKGCWLDDINCYDRTDCVETKENPQVYFCCCEGNMCNEKFSYFPEMEVTQPTS |
| 7 | GAILGRSETQECLFYNANWELERTNQTGVEPCEGEKDKRLHCFATWKNISGSIEIV |
| | KKGCWLDDINCYDRTDCVETKENPQVYFCCCEGNMCNEKFSYFPEMEVTQPTS |
| 8 | GAILGRSETQECLFYNANWELDRTNQTGVERCEGEKDKRLHCFATWKNISGSIEIV |
| | KKGCWLDDINCYDRTDCVETKENPQVYFCCCEGNMCNEKFSYFPEMEVTQPTS |
| 9 | GAILGRSETQECLFYNANWELDRTNQTGVEPCEGEKDKRLHCYATWKNISGSIEIV |
| | KKGCWLDDINCYDRTDCVETKENPQVYFCCCEGNMCNEKFSYFPEMEVTQPTS |
| 10 | GAILGRSETQECLFYNANWELDRTNQTGVEPCEGEKDKRLHCFATWRNISGSIEIV |
| | KKGCWLDDINCYDRTDCVETKENPQVYFCCCEGNMCNEKFSYFPEMEVTQPTS |
| 11 | GAILGRSETQECLFYNANWELDRTNQTGVEPCEGEKDKRLHCFATWKNISGSIEIV |
| | KKGCWLDDFNCYDRTDCVETKENPQVYFCCCEGNMCNEKFSYFPEMEVTQPTS |
| 12 | GAILGRSETQECLFYNANWELDRTNQTGVEPCEGEKDKRLHCFATWKNISGSIEIV |
| | KKGCWLDDINCYDRTDCVETEENPQVYFCCCEGNMCNEKFSYFPEMEVTQPTS |
| 13 | GAILGRSETQECLFYNANWELERTNQTGVERCEGEKDKRLHCFATWKNISGSIEIV |
| | KKGCWLDDINCYDRTDCVETKENPQVYFCCCEGNMCNEKFSYFPEMEVTQPTS |
| 14 | GAILGRSETQECLFYNANWELERTNQTGVEPCEGEKDKRLHCYATWKNISGSIEIV |
| | KKGCWLDDINCYDRTDCVETKENPQVYFCCCEGNMCNEKFSYFPEMEVTQPTS |
| 15 | GAILGRSETQECLFYNANWELERTNQTGVEPCEGEKDKRLHCFATWRNISGSIEIV |
| | KKGCWLDDINCYDRTDCVETKENPQVYFCCCEGNMCNEKFSYFPEMEVTQPTS |
| 16 | GAILGRSETQECLFYNANWELERTNQTGVEPCEGEKDKRLHCFATWKNISGSIEIV |
| | KKGCWLDDFNCYDRTDCVETKENPQVYFCCCEGNMCNEKFSYFPEMEVTQPTS |
| 17 | GAILGRSETQECLFYNANWELERTNQTGVEPCEGEKDKRLHCFATWKNISGSIEIV |
| | KKGCWLDDINCYDRTDCVETEENPQVYFCCCEGNMCNEKFSYFPEMEVTQPTS |
| 18 | GAILGRSETQECLFYNANWELDRTNQTGVERCEGEKDKRLHCYATWKNISGSIEIV |
| | KKGCWLDDINCYDRTDCVETKENPQVYFCCCEGNMCNEKFSYFPEMEVTQPTS |
| 19 | GAILGRSETQECLFYNANWELDRTNQTGVERCEGEKDKRLHCFATWRNISGSIEIV |
| | KKGCWLDDINCYDRTDCVETKENPQVYFCCCEGNMCNEKFSYFPEMEVTQPTS |
| 20 | GAILGRSETQECLFYNANWELDRTNQTGVERCEGEKDKRLHCFATWKNISGSIEIV |
| | KKGCWLDDFNCYDRTDCVETKENPQVYFCCCEGNMCNEKFSYFPEMEVTQPTS |
| 21 | GAILGRSETQECLFYNANWELDRTNQTGVERCEGEKDKRLHCFATWKNISGSIEIV |
| | KKGCWLDDINCYDRTDCVETEENPQVYFCCCEGNMCNEKFSYFPEMEVTQPTS |
| 22 | GAILGRSETQECLFYNANWELDRTNQTGVEPCEGEKDKRLHCYATWRNISGSIEIV |
| | KKGCWLDDINCYDRTDCVETKENPQVYFCCCEGNMCNEKFSYFPEMEVTQPTS |
| 23 | GAILGRSETQECLFYNANWELDRTNQTGVEPCEGEKDKRLHCYATWKNISGSIEIV |
| | KKGCWLDDFNCYDRTDCVETKENPQVYFCCCEGNMCNEKFSYFPEMEVTQPTS |
| 24 | GAILGRSETQECLFYNANWELDRTNQTGVEPCEGEKDKRLHCYATWKNISGSIEIV |
| | KKGCWLDDINCYDRTDCVETEENPQVYFCCCEGNMCNEKFSYFPEMEVTQPTS |
| 25 | GAILGRSETQECLFYNANWELDRTNQTGVEPCEGEKDKRLHCFATWRNISGSIEIV |
| | KKGCWLDDFNCYDRTDCVETKENPQVYFCCCEGNMCNEKFSYFPEMEVTQPTS |
| 26 | GAILGRSETQECLFYNANWELDRTNQTGVEPCEGEKDKRLHCFATWRNISGSIEIV |
| | KKGCWLDDINCYDRTDCVETEENPQVYFCCCEGNMCNEKFSYFPEMEVTQPTS |
| 27 | GAILGRSETQECLFYNANWELDRTNQTGVEPCEGEKDKRLHCFATWKNISGSIEIV |
| | KKGCWLDDFNCYDRTDCVETEENPQVYFCCCEGNMCNEKFSYFPEMEVTQPTS |
| 28 | GAILGRSETQECLFYNANWELERTNQTGVERCEGEKDKRLHCYATWKNISGSIEIV |
| | KKGCWLDDINCYDRTDCVETKENPQVYFCCCEGNMCNEKFSYFPEMEVTQPTS |
| 29 | GAILGRSETQECLFYNANWELERTNQTGVERCEGEKDKRLHCFATWRNISGSIEIV |
| | KKGCWLDDINCYDRTDCVETKENPQVYFCCCEGNMCNEKFSYFPEMEVTQPTS |
| 30 | GAILGRSETQECLFYNANWELERTNQTGVERCEGEKDKRLHCFATWKNISGSIEIV |
| | KKGCWLDDFNCYDRTDCVETKENPQVYFCCCEGNMCNEKFSYFPEMEVTQPTS |
| 31 | GAILGRSETQECLFYNANWELERTNQTGVERCEGEKDKRLHCFATWKNISGSIEIV |
| | KKGCWLDDINCYDRTDCVETEENPQVYFCCCEGNMCNEKFSYFPEMEVTQPTS |
| 32 | GAILGRSETQECLFYNANWELERTNQTGVEPCEGEKDKRLHCYATWRNISGSIEIV |
| | KKGCWLDDINCYDRTDCVETKENPQVYFCCCEGNMCNEKFSYFPEMEVTQPTS |
| 33 | GAILGRSETQECLFYNANWELERTNQTGVEPCEGEKDKRLHCYATWKNISGSIEIV |
| | KKGCWLDDFNCYDRTDCVETKENPQVYFCCCEGNMCNEKFSYFPEMEVTQPTS |
| 34 | GAILGRSETQECLFYNANWELERTNQTGVEPCEGEKDKRLHCYATWKNISGSIEIV |
| | KKGCWLDDINCYDRTDCVETEENPQVYFCCCEGNMCNEKFSYFPEMEVTQPTS |
| 35 | GAILGRSETQECLFYNANWELERTNQTGVEPCEGEKDKRLHCFATWRNISGSIEIV |
| | KKGCWLDDFNCYDRTDCVETKENPQVYFCCCEGNMCNEKFSYFPEMEVTQPTS |
| 36 | GAILGRSETQECLFYNANWELERTNQTGVEPCEGEKDKRLHCFATWRNISGSIEIV |
| | KKGCWLDDINCYDRTDCVETEENPQVYFCCCEGNMCNEKFSYFPEMEVTQPTS |
| 37 | GAILGRSETQECLFYNANWELERTNQTGVEPCEGEKDKRLHCFATWKNISGSIEIV |
| | KKGCWLDDFNCYDRTDCVETEENPQVYFCCCEGNMCNEKFSYFPEMEVTQPTS |
| 38 | GAILGRSETQECLFYNANWELDRTNQTGVERCEGEKDKRLHCYATWRNISGSIEIV |
| | KKGCWLDDINCYDRTDCVETKENPQVYFCCCEGNMCNEKFSYFPEMEVTQPTS |
| 39 | GAILGRSETQECLFYNANWELDRTNQTGVERCEGEKDKRLHCYATWKNISGSIEIV |
| | KKGCWLDDFNCYDRTDCVETKENPQVYFCCCEGNMCNEKFSYFPEMEVTQPTS |
| 40 | GAILGRSETQECLFYNANWELDRTNQTGVERCEGEKDKRLHCYATWKNISGSIEIV |
| | KKGCWLDDINCYDRTDCVETEENPQVYFCCCEGNMCNEKFSYFPEMEVTQPTS |
| 41 | GAILGRSETQECLFYNANWELDRTNQTGVERCEGEKDKRLHCFATWRNISGSIEIV |
| | KKGCWLDDFNCYDRTDCVETKENPQVYFCCCEGNMCNEKFSYFPEMEVTQPTS |
| 42 | GAILGRSETQECLFYNANWELDRTNQTGVERCEGEKDKRLHCFATWRNISGSIEIV |
| | KKGCWLDDINCYDRTDCVETEENPQVYFCCCEGNMCNEKFSYFPEMEVTQPTS |
| 43 | GAILGRSETQECLFYNANWELDRTNQTGVERCEGEKDKRLHCFATWKNISGSIEIV |
| | KKGCWLDDFNCYDRTDCVETEENPQVYFCCCEGNMCNEKFSYFPEMEVTQPTS |
| 44 | GAILGRSETQECLFYNANWELDRTNQTGVEPCEGEKDKRLHCYATWRNISGSIEIV |
| | KKGCWLDDFNCYDRTDCVETKENPQVYFCCCEGNMCNEKFSYFPEMEVTQPTS |
| 45 | GAILGRSETQECLFYNANWELDRTNQTGVEPCEGEKDKRLHCYATWRNISGSIEIV |
| | KKGCWLDDINCYDRTDCVETEENPQVYFCCCEGNMCNEKFSYFPEMEVTQPTS |
| 46 | GAILGRSETQECLFYNANWELDRTNQTGVEPCEGEKDKRLHCYATWKNISGSIEIV |
| | KKGCWLDDFNCYDRTDCVETEENPQVYFCCCEGNMCNEKFSYFPEMEVTQPTS |
| 47 | GAILGRSETQECLFYNANWELDRTNQTGVEPCEGEKDKRLHCFATWRNISGSIEIV |
| | KKGCWLDDFNCYDRTDCVETEENPQVYFCCCEGNMCNEKFSYFPEMEVTQPTS |
| 48 | GAILGRSETQECLFYNANWELERTNQTGVERCEGEKDKRLHCYATWRNISGSIEIV |
| | KKGCWLDDINCYDRTDCVETKENPQVYFCCCEGNMCNEKFSYFPEMEVTQPTS |
| 49 | GAILGRSETQECLFYNANWELERTNQTGVERCEGEKDKRLHCYATWKNISGSIEIV |
| | KKGCWLDDFNCYDRTDCVETKENPQVYFCCCEGNMCNEKFSYFPEMEVTQPTS |
| 50 | GAILGRSETQECLFYNANWELERTNQTGVERCEGEKDKRLHCYATWKNISGSIEIV |
| | KKGCWLDDINCYDRTDCVETEENPQVYFCCCEGNMCNEKFSYFPEMEVTQPTS |
| 51 | GAILGRSETQECLFYNANWELERTNQTGVERCEGEKDKRLHCFATWRNISGSIEIV |
| | KKGCWLDDFNCYDRTDCVETKENPQVYFCCCEGNMCNEKFSYFPEMEVTQPTS |
| 52 | GAILGRSETQECLFYNANWELERTNQTGVERCEGEKDKRLHCFATWRNISGSIEIV |
| | KKGCWLDDINCYDRTDCVETEENPQVYFCCCEGNMCNEKFSYFPEMEVTQPTS |
| 53 | GAILGRSETQECLFYNANWELERTNQTGVERCEGEKDKRLHCFATWKNISGSIEIV |
| | KKGCWLDDFNCYDRTDCVETEENPQVYFCCCEGNMCNEKFSYFPEMEVTQPTS |
| 54 | GAILGRSETQECLFYNANWELERTNQTGVEPCEGEKDKRLHCYATWRNISGSIEIV |
| | KKGCWLDDFNCYDRTDCVETKENPQVYFCCCEGNMCNEKFSYFPEMEVTQPTS |
| 55 | GAILGRSETQECLFYNANWELERTNQTGVEPCEGEKDKRLHCYATWRNISGSIEIV |
| | KKGCWLDDINCYDRTDCVETEENPQVYFCCCEGNMCNEKFSYFPEMEVTQPTS |
| 56 | GAILGRSETQECLFYNANWELERTNQTGVEPCEGEKDKRLHCYATWKNISGSIEIV |
| | KKGCWLDDFNCYDRTDCVETEENPQVYFCCCEGNMCNEKFSYFPEMEVTQPTS |
| 57 | GAILGRSETQECLFYNANWELERTNQTGVEPCEGEKDKRLHCFATWRNISGSIEIV |
| | KKGCWLDDFNCYDRTDCVETEENPQVYFCCCEGNMCNEKFSYFPEMEVTQPTS |
| 58 | GAILGRSETQECLFYNANWELDRTNQTGVERCEGEKDKRLHCYATWRNISGSIEIV |
| | KKGCWLDDFNCYDRTDCVETKENPQVYFCCCEGNMCNEKFSYFPEMEVTQPTS |
| 59 | GAILGRSETQECLFYNANWELDRTNQTGVERCEGEKDKRLHCYATWRNISGSIEIV |
| | KKGCWLDDINCYDRTDCVETEENPQVYFCCCEGNMCNEKFSYFPEMEVTQPTS |
| 60 | GAILGRSETQECLFYNANWELDRTNQTGVERCEGEKDKRLHCYATWKNISGSIEIV |
| | KKGCWLDDFNCYDRTDCVETEENPQVYFCCCEGNMCNEKFSYFPEMEVTQPTS |
| 61 | GAILGRSETQECLFYNANWELDRTNQTGVERCEGEKDKRLHCFATWRNISGSIEIV |
| | KKGCWLDDFNCYDRTDCVETEENPQVYFCCCEGNMCNEKFSYFPEMEVTQPTS |
| 62 | GAILGRSETQECLFYNANWELDRTNQTGVEPCEGEKDKRLHCYATWRNISGSIEIV |
| | KKGCWLDDFNCYDRTDCVETEENPQVYFCCCEGNMCNEKFSYFPEMEVTQPTS |
| 63 | GAILGRSETQECLFYNANWELERTNQTGVERCEGEKDKRLHCYATWRNISGSIEIV |
| | KKGCWLDDFNCYDRTDCVETKENPQVYFCCCEGNMCNEKFSYFPEMEVTQPTS |
| 64 | GAILGRSETQECLFYNANWELERTNQTGVERCEGEKDKRLHCYATWRNISGSIEIV |
| | KKGCWLDDINCYDRTDCVETEENPQVYFCCCEGNMCNEKFSYFPEMEVTQPTS |
| 65 | GAILGRSETQECLFYNANWELERTNQTGVERCEGEKDKRLHCYATWKNISGSIEIV |
| | KKGCWLDDFNCYDRTDCVETEENPQVYFCCCEGNMCNEKFSYFPEMEVTQPTS |
| 66 | GAILGRSETQECLFYNANWELERTNQTGVERCEGEKDKRLHCFATWRNISGSIEIV |
| | KKGCWLDDFNCYDRTDCVETEENPQVYFCCCEGNMCNEKFSYFPEMEVTQPTS |
| 67 | GAILGRSETQECLFYNANWELDRTNQTGVERCEGEKDKRLHCYATWRNISGSIEIV |
| | KKGCWLDDFNCYDRTDCVETEENPQVYFCCCEGNMCNEKFSYFPEMEVTQPTS |
| 68 | GAILGRSETQECLFYNANWELERTNQTGVEPCEGEKDKRLHCYATWRNISGSIEIV |
| | KKGCWLDDFNCYDRTDCVETEENPQVYFCCCEGNMCNEKFSYFPEMEVTQPTS |
| 69 | GAILGRSETQECLFYNANWELERTNQTGVERCEGEKDKRLHCYATWRNISGSIEIV |
| | KKGCWLDDFNCYDRTDCVETEENPQVYFCCCEGNMCNEKFSYFPEMEVTQPTS |
| 70 | GAILGRSETQECLYYNANWELERTNQTGVERCEGEQDKRLHCYATWRNISGSIEIV |
| | KKGCWLDDFNCYDRTDCVETEENPQVYFCCCEGNMCNEKFSYFPEMEVTQPTS |
| 71 | GAILGRSETQECLFYNANWELERTNQTGVERCEGEKDKRLHCYATWRNISGSIEIV |
| | KKGCWLDDFNCYDRTDCVETEENPQVYFCCCEGNMCNEKFSYFPEMEVTQPTSN |
| | P |
| 72 | GAILGRSETQECLFYNANWELERTNQTGVERCEGEKDKRLHCYATWRNISGSIEIV |
| | KKGCWLDDFNCYDRTDCVETEENPQVYFCCCEGNMCNEKFSYFPEMEVTQPTSN |
| | PVTPK |

In some embodiments, an ActRIIA ligand trap including an extracellular ActRIIA variant (e.g., any one of SEQ ID NOs: 1-72 (e.g., SEQ ID NOs: 6-72)) has amino acid K at position X₁₇. Altering the amino acid at position X₁₇ can result in reduced activity. For example, an ActRIIA variant having the sequence GAILGRSETQECLFYNANWELERTNQTGVERCEGEKDKRLHCYATWRNISGSIEIVAKGCWLDDFNCYD RTDCVETEENPQVYFCCCEGNMCNEKFSYFPEMEVTQPTS (SEQ ID NO: 85) has reduced activity in vivo, indicating that the substitution of A for K at X₁₇ is not tolerated.

In some embodiments, an ActRIIA ligand trap including an extracellular ActRIIA variant (e.g., any one of SEQ ID NOs: 1-72 (e.g., SEQ ID NOs: 6-72)) with the sequence TEEN (SEQ ID NO: 76) at positions X₂₃, X₂₄, X₂₅, and X₂₆ can have a substitution of the amino acid K for the amino acid E at position X₂₄. In some embodiments, an ActRIIA ligand trap including an extracellular ActRIIA variant (e.g., any one of SEQ ID NOs: 1-72 (e.g., SEQ ID NOs: 6-72)) with the sequence TKEN (SEQ ID NO: 77) at positions X₂₃, X₂₄, X₂₅, and X₂₆ can have a substitution of the amino acid E for the amino acid K at position X₂₄. ActRIIA variants having the sequence TEEN (SEQ ID NO: 76) or TKEN (SEQ ID NO: 77) at positions X₂₃, X₂₄, X₂₅, and X₂₆ have reduced or weak binding to BMP9 (e.g., reduced binding to BMP9 compared to BMP9 binding of wild-type ActRIIA).

In some embodiments, an ActRIIA ligand trap including an extracellular ActRIIA variant (e.g., any one of SEQ ID NOs: 1-70 (e.g., SEQ ID NOs: 6-70)) may further include a C-terminal extension (e.g., one more additional amino acids at the C-terminus). The C-terminal extension may correspond to sequence from the same position in wild-type ActRIIA. In some embodiments, the C-terminal extension is amino acid sequence NP. For example, a sequence including the C-terminal extension NP is SEQ ID NO: 71 (e.g., SEQ ID NO: 69 with a C-terminal extension of NP). In some embodiments, the C-terminal extension is amino acid sequence NPVTPK (SEQ ID NO: 78). For example, a sequence including the C-terminal extension NPVTPK (SEQ ID NO: 78) is SEQ ID NO: 72 (e.g., SEQ ID NO: 69 with a C-terminal extension of NPVTPK (SEQ ID NO: 78)). The C-terminal extension can add one or more amino acids at the C-terminus (e.g., 1, 2, 3, 4, 5, 6 or more additional amino acids).

In some embodiments, an ActRIIA ligand trap including an extracellular ActRIIA variant may further include a moiety (e.g., Fc domain monomer, an Fc domain, an albumin-binding peptide, a fibronectin domain, or a human serum albumin), which may be fused to the N- or C-terminus (e.g., C-terminus) of the extracellular ActRIIA variant by way of a linker or other covalent bonds. A polypeptide including an extracellular ActRIIA variant fused to an Fc domain monomer may form a dimer (e.g., homodimer or heterodimer) through the interaction between two Fc domain monomers, which combine to form an Fc domain in the dimer.

Furthermore, in some embodiments, an ActRIIA ligand trap described herein (e.g., an ActRIIA variant-Fc fusion protein) has a serum half-life of at least 7 days in humans. The ActRIIA ligand trap may bind to activin A with a Ko of 10 pM or higher. In some embodiments, the ActRIIA ligand trap does not bind to BMP9 or activin A. In some embodiments, the ActRIIA ligand trap binds to activin A, activin B, and/or myostatin and exhibits reduced (e.g., weak) binding to BMP9 (e.g., reduced BMP9 binding compared to BMP9 binding of wild-type ActRIIA). In some embodiments, the ActRIIA ligand trap that has reduced or weak binding to BMP9 has the sequence TEEN (SEQ ID NO: 76) or TKEN (SEQ ID NO: 77) at positions X₂₃, X₂₄, X₂₅, and X₂₆. In some embodiments, the ActRIIA ligand trap does not substantially bind to human BMP9.

In some embodiments, the ActRIIA ligand trap may bind to human activin A with a K_{D} of about 800 pM or less (e.g., a K_{D} of about 800, 700, 600, 500, 400, 300, 200, 100, 90, 80, 70, 60, 50, 40, 30, 20, 10, 9, 8, 7, 6, 5, 4, 3, 2, or 1 pM or less, e.g., a K_{D} of between about 800 pM and about 200 pM). In some embodiments, the ActRIIA ligand trap may bind to human activin B with a Ko of 800 pM or less (e.g., a K_{D} of about 800, 700, 600, 500, 400, 300, 200, 100, 90, 80, 70, 60, 50, 40, 30, 20, 10, 9, 8, 7, 6, 5, 4, 3, 2, or 1 pM or less, e.g., a K_{D} of between about 800 PM and about 200 pM) The ActRIIA ligand trap may also bind to growth and differentiation factor 11 (GDF-11) with a K_{D} of approximately 5 pM or higher (e.g., a K_{D} of about 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, or 200 pM or higher).

In some embodiments, the ActRIIA ligand trap is sotatercept (also known as ACE-011). Additional ActRIIA ligand traps that may be used in the methods described herein are described in International Patent Application Publication No. WO2007062188 and U.S. Pat. Nos. 7,709,605, 9,138,459, 7,612,041, 8,067,360, 8,629,109, 9,572,865, 9,163,075, 10,071,135, and 7,951,771, each of which is incorporated herein by reference.

In some embodiments, the ActRII ligand trap is an ActRIIB ligand trap. The ActRIIB ligand trap may contain an extracellular portion of wild-type ActRIIB (e.g., human or murine ActRIIB) or may contain an extracellular portion of wild-type ActRIIB that contains one or more amino acid substitutions relative to the wild-type human extracellular ActRIIB. The wild-type amino acid sequence of the extracellular portion of human ActRIIB is shown below.

Human ActRIIB, extracellular portion (SEQ ID NO:
74):
GRGEAETRECIYYNANWELERTNQSGLERCEGEQDKRLHCYASWRNSSGT
IELVKKGCWLDDFNCYDRQECVATEENPQVYFCCCEGNFCNERFTHLPEA
GGPEVTYEPPPTAPT

An ActRIIB ligand trap may contain the sequence of SEQ ID NO: 74 or a variant thereof that contains one or more amino acid substitutions. In some embodiments, the ActRIIB ligand trap contains a portion of SEQ ID NO: 74 (e.g., a contiguous portion that is shortened by the removal of amino acids from the N-terminus, C-terminus, or both) or a variant thereof that contains one or more amino acid substitutions. For example, the ActRIIB ligand trap can include the sequence of SEQ ID NO: 74 with an L60D substitution. In another example, the ActRIIB ligand trap can include the sequence of SEQ ID NO: 74 with a substitution at position E9 (e.g., an E9W, E9A, E9F, E9Q, E9V, E9I, E9L, E9M, E9K, E9H, or E9Y substitution), an S25T substitution, and/or an R45A substitution. In some embodiments, the ActRIIB ligand trap is BIIB110 (previously known as ALG-801), ALG-802, luspatercept (REBLOZYL®, also known as ACE-536), Ramatercept (also known as ACE-031), or ACE-2494. Additional ActRIIB ligand traps that may be used in the methods described herein include those described in International Patent Application Publication Nos. WO2010/062383, WO2015/192127, WO2019140283, and WO2021189010; US Patent Application Publication Nos. US20110250198 and US20200407415; and U.S. Pat. Nos. 10,913,782, 8,058,229, 8,216,997, 8,703,927, 9,439,945, 9,932,379, 10,131,700, 10,689,427, 10,889,626, 10,829,532, 10,829,533, 8,361,957, 9,505,813, 10,377,996, 9,617,319, 8,710,016, 7,709,605, 8,252,900, 7,842,663, 8,343,933, 9,399,669, 10,259,861, 8,138,142, 8,178,488, 8,293,881, 9,181,533, 9,745,559, 10,358,633, 11,066,654, 9,610,327, 9,284,364, 8,067,562, 8,614,292, 7,947,646, 8,716,459, 8,501,678, 8,999,917, 9,447,165, 9,809,638, 10,407,487, 8,410,043, 9,273,114, and 10,308,704, each of which is incorporated herein by reference.

In some embodiments, the ActRIIB ligand trap contains an ActRIIB variant having the sequence of SEQ ID NO: 730 shown in Table 13.

TABLE 13
Amino acid substitutions in an extracellular
ActRIIB variant having a sequence of SEQ ID NO:
730

GRGEAETRECX₁X₂YNANWEX₃X₄RTNQX₅GX₆EX₇CX₈GX₉X₁₀DKR
X₁₁HCX₁₂ASWX₁₃NX₁₄X₁₅GX₁₆X₁₇EX₁₈VKX₁₉GCWLDDX₂₀NCYD
RX₂₁X₂₂CVAX₂₃X₂₄X₂₅X₂₆PX₂₇VYFCCCEGNX₂₈CNERFTHLPEAG
GPEVTYEPPPTAPT (SEQ ID NO: 730)

| | | | |
|---|---|---|---|
| X₁ | I or L | X₁₅ | S or T |
| X₂ | F or Y | X₁₆ | S or T |
| X₃ | L or K | X₁₇ | I or L |
| X₄ | D or E | X₁₈ | I or L |
| X₅ | T or S | X₁₉ | K or Q |
| X₆ | L or V | X₂₀ | F or I |
| X₇ | P or R | X₂₁ | Q, T, or D |
| X₈ | Y or E | X₂₂ | E, D, or T |
| X₉ | D or E | X₂₃ | K or T |
| X₁₀ | K or Q | X₂₄ | K or E |
| X₁₁ | R or L | X₂₅ | D or E |
| X₁₂ | Y or F | X₂₆ | S or N |
| X₁₃ | R or K | X₂₇ | E or Q |
| X₁₄ | S or I | X₂₈ | F or M |

In some embodiments, the ActRIIB variant has the sequence of any one of SEQ ID NOs: 731-744 (Table 14).

TABLE 14
Extracellular ActRIIB variants having the sequences of SEQ ID NOs: 731-744

| SEQ ID NO | Amino Acid Sequence |
|---|---|
| 731 | GRGEAETRECIFYNANWEKDRTNQSGLEPCYGDQDKRRHCFASWKNSSGTIELVKQGC |
| | WLDDINCYDRQECVAKKDSPEVYFCCCEGNFCNERFTHLPEAGGPEVTYEPPPTAPT |
| 732 | GRGEAETRECIYYNANWELDRTNQSGLERCEGEQDKRLHCYASWRNSSGTIELVKKGC |
| | WLDDINCYDRQECVATKENPQVYFCCCEGNFCNERFTHLPEAGGPEVTYEPPPTAPT |
| 733 | GRGEAETRECIYYNANWELERTNQSGLERCEGEQDKRLHCYASWRNSSGTIELVKKGC |
| | WLDDFNCYDRQECVAKKDSPEVYFCCCEGNFCNERFTHLPEAGGPEVTYEPPPTAPT |
| 734 | GRGEAETRECIYYNANWELERTNQTGLERCEGEQDKRLHCYASWRNISGTIELVKKGC |
| | WLDDFNCYDRQECVAKKDSPEVYFCCCEGNFCNERFTHLPEAGGPEVTYEPPPTAPT |
| 735 | GRGEAETRECIYYNANWELERTNQTGLERCEGEQDKRLHCYASWRNITGTIELVKKGC |
| | WLDDFNCYDRQECVAKKDSPEVYFCCCEGNFCNERFTHLPEAGGPEVTYEPPPTAPT |
| 736 | GRGEAETRECIYYNANWELERTNQSGLEPCEGEQDKRLHCYASWRNSSGTIELVKKGC |
| | WLDDFNCYDRQECVAKKDSPEVYFCCCEGNFCNERFTHLPEAGGPEVTYEPPPTAPT |
| 737 | GRGEAETRECIYYNANWELERTNQSGLERCYGDKDKRLHCYASWRNSSGTIELVKKGC |
| | WLDDFNCYDRQECVAKKDSPEVYFCCCEGNFCNERFTHLPEAGGPEVTYEPPPTAPT |
| 738 | GRGEAETRECIYYNANWELERTNQSGLERCEGEQDKRLHCFASWKNSSGTIELVKQGC |
| | WLDDFNCYDRQECVAKKDSPEVYFCCCEGNFCNERFTHLPEAGGPEVTYEPPPTAPT |
| 739 | GRGEAETRECIFYNANWEKDRTNQSGLERCEGEQDKRLHCYASWRNSSGTIELVKKGC |
| | WLDDFNCYDRQECVAKKDSPEVYFCCCEGNFCNERFTHLPEAGGPEVTYEPPPTAPT |
| 740 | GRGEAETRECIYYNANWELERTNQSGLERCYGDQDKRRHCYASWRNSSGTIELVKKGC |
| | WLDDFNCYDRQECVAKKDSPEVYFCCCEGNFCNERFTHLPEAGGPEVTYEPPPTAPT |
| 741 | GRGEAETRECLYYNANWELERTNQSGVERCEGEKDKRLHCYASWRNSSGSLEIVKKGC |
| | WLDDFNCYDRTDCVATEENPQVYFCCCEGNMCNERFTHLPEAGGPEVTYEPPPTAPT |
| 742 | GRGEAETRECLYYNANWELERTNQSGVERCEGEKDKRLHCYASWRNSSGSLEIVKKGC |
| | WLDDFNCYDRDTCVATEENPQVYFCCCEGNMCNERFTHLPEAGGPEVTYEPPPTAPT |
| 743 | GRGEAETRECLYYNANWELERTNQSGVERCEGEKDKRLHCYASWRNSSGSLEIVKKGC |
| | WLDDFNCYDRTDCVATKENPQVYFCCCEGNMCNERFTHLPEAGGPEVTYEPPPTAPT |
| 744 | GRGEAETRECLYYNANWELERTNQSGVERCEGEKDKRLHCYASWRNSSGSLEIVKKGC |
| | WLDDFNCYDRDTCVATKENPQVYFCCCEGNMCNERFTHLPEAGGPEVTYEPPPTAPT |

In some embodiments, the extracellular ActRIIB variant has an N-terminal truncation of 1-7 amino acids (e.g., 1, 2, 3, 4, 5, 6, or 7 amino acids). An N-terminal truncation can be produced by removing 1-7 amino acids from the N-terminus of an of an ActRIIB variant shown in Tables 13 and 14. The N-terminal truncation can remove amino acids up two to amino acids before the first cysteine (e.g., the two amino acids before the first cysteine (RE) are retained in the N-terminally truncated ActRIIB variants). Additional ActRIIB variants having an N-terminal truncation are provided below:

(SEQ ID NO: 745)
ETRECIYYNANWELERTNQSGLERCYGDKDKRRHCYASWRNSSGTIELVK
KGCWLDDFNCYDRQECVATEENPQVYFCCCEGNFCNERFTHLPEAGGPEV
TYEPPPTAPT
(SEQ ID NO: 746)
ETRECIYYNANWELERTNQSGLERCEGDQDKRLHCYASWRNSSGTIELVK
KGCWLDDINCYDRQECVATKENPQVYFCCCEGNFCNERFTHLPEAGGPEV
TYEPPPTAPT
(SEQ ID NO: 747)
ETRECIYYNANWELERTNQSGLERCEGEQDKRLHCYASWRNSSGTIELVK
KGCWDDDFNCYDRQECVATEENPQVYFCCCEGNFCNERFTHLPEAGGPEV
TYEPPPT
(SEQ ID NO: 748)
ETRWCIYYNANWELERTNQSGLERCEGEQDKRLHCYASWRNSSGTIELVK
KGCWLDDFNCYDRQECVATEENPQVYFCCCEGNFCNERFTHLPEAGGPEV
TYEPPPTAPT
(SEQ ID NO: 749)
ETRWCIYYNANWELERTNQTGLERCEGEQDKRLHCYASWRNSSGTIELVK
KGCWLDDFNCYDRQECVATEENPQVYFCCCEGNFCNERFTHLPEAGGPEV
TYEPPPTAPT
(SEQ ID NO: 750)
ETRYCIYYNANWELERTNQTGLERCEGEQDKRLHCYASWRNSSGTIELVK
KGCWLDDFNCYDRQECVATEENPQVYFCCCEGNFCNERFTHLPEAGGPEV
TYEPPPTAPT

In some embodiments, an ActRIIB ligand trap including an ActRIIB variant may further include a moiety (e.g., Fc domain monomer, an Fc domain, an albumin-binding peptide, a fibronectin domain, or a human serum albumin), which may be fused to the N- or C-terminus (e.g., C-terminus) of the extracellular ActRIIB variant by way of a linker or other covalent bonds. An ActRIIB ligand trap including an extracellular ActRIIB variant fused to an Fc domain monomer may form a dimer (e.g., homodimer or heterodimer) through the interaction between two Fc domain monomers, which combine to form an Fc domain in the dimer.

In some embodiments, the ActRII ligand trap is an ActRII chimera ligand trap. The ActRII chimera ligand traps contain portions of extracellular ActRIIA (e.g., human ActRIIA) and extracellular ActRIIB (e.g., human ActRIIB). In some embodiments, the ActRII chimera ligand traps contain an N-terminal portion of extracellular ActRIIB (SEQ ID NO: 74 shown above) joined to a C-terminal portion of extracellular ActRIIA (SEQ ID NO: 73 shown above) such that the sequences are contiguous (e.g., the ActRIIA sequence continues where the ActRIIB sequence left off, starting with the next the amino acid located in the corresponding position of ActRIIA). In some embodiments, the N-terminus of the ActRII chimera included in the ActRII chimera ligand trap includes the six amino acids found at the N-terminus of extracellular ActRIIA joined to the fifth amino acid of extracellular ActRIIB. In some embodiments, the N-terminus of the ActRII chimera included in the ActRII chimera ligand trap begins with the first amino acid located at the N-terminus of extracellular ActRIIB. In some embodiments, the N-terminus of the ActRII chimera included in the ActRII chimera ligand trap includes the first ten amino acids found at the N-terminus of extracellular ActRIIA joined to the ninth amino acid of extracellular ActRIIB. The extracellular ActRII chimera included in the ActRII chimera ligand trap may also include one or more amino acid substitutions in the portion of the chimera that corresponds to the sequence of ActRIIB compared to wild-type extracellular ActRIIB (e.g., SEQ ID NO: 74 shown above), and one or more amino acid substitutions in the portion of the chimera that corresponds to the sequence of ActRIIA compared to wild-type extracellular ActRIIA (e.g., SEQ ID NO: 73 shown above). Amino acid substitutions at 9 different positions may be introduced into an extracellular ActRII chimera (Table 15). An extracellular ActRII chimera may have one or more (e.g., 1, 2, 3, 4, 5, 6, 7, 8, or 9) amino acid substitutions relative the sequence of a wild-type sequence (e.g., relative to the sequence of wild-type extracellular ActRIIB (SEQ ID NO: 74) if the portion of the chimera corresponds to a region of wild-type extracellular ActRIIB, or relative to the sequence of wild-type extracellular ActRIIA (SEQ ID NO: 73) if the portion of the chimera corresponds to a region of wild-type extracellular ActRIIA). The positions at which amino acid substitutions may be made, as well as the amino acids that may be substituted at these positions, are listed in Table 15. ActRII chimera ligand traps that may be used in the methods described herein include those described in International Patent Application Publication No. WO2021189019A1, the disclosure of which is incorporated herein by reference.

TABLE 15
Amino acid substitutions in an extracellular ActRII chimera having a sequence of any
one of SEQ ID NOs: 751-771

GAILGRAETRECIYYNANWELERTNQSGLERCEGEQX₁KRRHCFATWKNISGSIEIVKQGCWLDDX₂X₃
CYDRTDCVX₄X₅X₆X₇X₈PX₉VYFCCCEGNMCNEKFSYFPEMEVTQPTS (SEQ ID NO: 751)
GAILGRAETRECIYYNANWELERTNQSGLERCEGEQX₁KRLHCFATWKNISGSIEIVKQGCWLDDX₂X₃
CYDRTDCVX₄X₅X₆X₇X₈PX₉VYFCCCEGNMCNEKFSYFPEMEVTQPTS (SEQ ID NO: 752)
GAILGRAETRECIYYNANWELERTNQSGLERCEGEQX₁KRLHCYASWKNISGSIEIVKQGCWLDDX₂X₃
CYDRTDCVX₄X₅X₆X₇X₈PX₉VYFCCCEGNMCNEKFSYFPEMEVTQPTS (SEQ ID NO: 753)
GAILGRAETRECIYYNANWELERTNQSGLERCEGEQX₁KRLHCYASWRNSSGSIEIVKQGCWLDDX₂
X₃CYDRTDCVX₄X₅X₆X₇X₈PX₉VYFCCCEGNMCNEKFSYFPEMEVTQPTS (SEQ ID NO: 754)
GAILGRAETRECIYYNANWELERTNQSGLERCEGEQX₁KRLHCYASWRNSSGTIEIVKQGCWLDDX₂X₃
CYDRTDCVX₄X₅X₆X₇X₈PX₉VYFCCCEGNMCNEKFSYFPEMEVTQPTS (SEQ ID NO: 755)
GAILGRAETRECIYYNANWELERTNQSGLERCEGEQX₁KRLHCYASWRNSSGTIELVKKGCWLDDX₂
X₃CYDRTDCVX₄X₅X₆X₇X₈PX₉VYFCCCEGNMCNEKFSYFPEMEVTQPTS (SEQ ID NO: 756)
GAILGRAETRECIYYNANWELERTNQSGLERCEGEQX₁KRLHCYASWRNSSGTIELVKKGCWLDDX₂
X₃CYDRQECVX₄X₅X₆X₇X₈PX₉VYFCCCEGNMCNEKFSYFPEMEVTQPTS (SEQ ID NO: 757)
GRGEAETRECIYYNANWELERTNQSGLERCEGEQX₁KRRHCFATWKNISGSIEIVKQGCWLDDX₂X₃C
YDRTDCVX₄X₅X₆X₇X₈PX₉VYFCCCEGNMCNEKFSYFPEMEVTQPTS (SEQ ID NO: 758)
GRGEAETRECIYYNANWELERTNQSGLERCEGEQX₁KRLHCFATWKNISGSIEIVKQGCWLDDX₂X₃C
YDRTDCVX₄X₅X₆X₇X₈PX₉VYFCCCEGNMCNEKFSYFPEMEVTQPTS (SEQ ID NO: 759)
GRGEAETRECIYYNANWELERTNQSGLERCEGEQX₁KRLHCYASWKNISGSIEIVKQGCWLDDX₂X₃C
YDRTDCVX₄X₅X₆X₇X₈PX₉VYFCCCEGNMCNEKFSYFPEMEVTQPTS (SEQ ID NO: 760)
GRGEAETRECIYYNANWELERTNQSGLERCEGEQX₁KRLHCYASWRNSSGSIEIVKQGCWLDDX₂X₃C
YDRTDCVX₄X₅X₆X₇X₈PX₉VYFCCCEGNMCNEKFSYFPEMEVTQPTS (SEQ ID NO: 761)
GRGEAETRECIYYNANWELERTNQSGLERCEGEQX₁KRLHCYASWRNSSGTIEIVKQGCWLDDX₂X₃C
YDRTDCVX₄X₅X₆X₇X₈PX₉VYFCCCEGNMCNEKFSYFPEMEVTQPTS (SEQ ID NO: 762)
GRGEAETRECIYYNANWELERTNQSGLERCEGEQX₁KRLHCYASWRNSSGTIELVKKGCWLDDX₂X₃
CYDRTDCVX₄X₅X₆X₇X₈PX₉VYFCCCEGNMCNEKFSYFPEMEVTQPTS (SEQ ID NO: 763)
GRGEAETRECIYYNANWELERTNQSGLERCEGEQX₁KRLHCYASWRNSSGTIELVKKGCWLDDX₂X₃
CYDRQECVX₄X₅X₆X₇X₈PX₉VYFCCCEGNMCNEKFSYFPEMEVTQPTS (SEQ ID NO: 764)
GAILGRSETQECIYYNANWELERTNQSGLERCEGEQX₁KRRHCFATWKNISGSIEIVKQGCWLDDX₂X₃
CYDRTDCVX₄X₅X₆X₇X₈PX₉VYFCCCEGNMCNEKFSYFPEMEVTQPTS (SEQ ID NO: 765)
GAILGRSETQECIYYNANWELERTNQSGLERCEGEQX₁KRLHCFATWKNISGSIEIVKQGCWLDDX₂X₃
CYDRTDCVX₄X₅X₆X₇X₈PX₉VYFCCCEGNMCNEKFSYFPEMEVTQPTS (SEQ ID NO: 766)
GAILGRSETQECIYYNANWELERTNQSGLERCEGEQX₁KRLHCYASWKNISGSIEIVKQGCWLDDX₂X₃
CYDRTDCVX₄X₅X₆X₇X₈PX₉VYFCCCEGNMCNEKFSYFPEMEVTQPTS (SEQ ID NO: 767)
GAILGRSETQECIYYNANWELERTNQSGLERCEGEQX₁KRLHCYASWRNSSGSIEIVKQGCWLDDX₂
X₃CYDRTDCVX₄X₅X₆X₇X₈PX₉VYFCCCEGNMCNEKFSYFPEMEVTQPTS (SEQ ID NO: 768)
GAILGRSETQECIYYNANWELERTNQSGLERCEGEQX₁KRLHCYASWRNSSGTIEIVKQGCWLDDX₂
X₃CYDRTDCVX₄X₅X₆X₇X₈PX₉VYFCCCEGNMCNEKFSYFPEMEVTQPTS (SEQ ID NO: 769)
GAILGRSETQECIYYNANWELERTNQSGLERCEGEQX₁KRLHCYASWRNSSGTIELVKKGCWLDDX₂
X₃CYDRTDCVX₄X₅X₆X₇X₈PX₉VYFCCCEGNMCNEKFSYFPEMEVTQPTS (SEQ ID NO: 770)
GAILGRSETQECIYYNANWELERTNQSGLERCEGEQX₁KRLHCYASWRNSSGTIELVKKGCWLDDX₂
X₃CYDRQECVX₄X₅X₆X₇X₈PX₉VYFCCCEGNMCNEKFSYFPEMEVTQPTS (SEQ ID NO: 771)

| | | | |
|---|---|---|---|
| X₁ | D or R | X₆ | E or K |
| X₂ | I, F, E, D, Y, S, N, Q, or T | X₇ | E or D |
| X₃ | N or T | X₈ | N or S |
| X₄ | A or E | X₉ | Q, E, K, R, D, or N |
| X₅ | T or K | | |

In some embodiments, in ActRII chimeras of SEQ ID NOs: 751-771 (shown in Table 15), X₁ is D, X₂ is I, F, or E, X₃ is N or T, X₄ is A or E, X₅ is T or K, X₆ is E or K, X₇ is E or D, X₈ is N or S, and X₉ is E or Q. In some embodiments, in the extracellular ActRII chimeras of SEQ ID NOs: 751-771, X₁ is D, X₂ is I or F, X₃ is N, X₄ is A or E, X₅ is T or K, X₆ is E or K, X₇ is E or D, X₈ is N or S, and X₉ is E or Q.

In some embodiments, ActRII chimera ligand trap contains the sequence of any one of SEQ ID NOs: 772-793 (Table 16).

TABLE 16
Extracellular ActRII chimeras having the sequences of SEQ ID NOs: 772-793

| SEQ ID NO: | Amino Acid Sequence |
|---|---|
| 772 | GAILGRAETRECIYYNANWELERTNQSGLERCEGEQDKRLHCYASWRNSSGTIEIV |
| | KQGCWLDDINCYDRTDCVATEENPQVYFCCCEGNMCNEKFSYFPEMEVTQPTS |
| 773 | GRGEAETRECIYYNANWELERTNQSGLERCEGEQDKRLHCYASWRNSSGTIEIVK |
| | QGCWLDDINCYDRTDCVEKKDSPEVYFCCCEGNMCNEKFSYFPEMEVTQPTS |
| 774 | GRGEAETRECIYYNANWELERTNQSGLERCEGEQDKRLHCYASWRNSSGTIEIVK |
| | QGCWLDDINCYDRTDCVEKKDSPQVYFCCCEGNMCNEKFSYFPEMEVTQPTS |
| 775 | GRGEAETRECIYYNANWELERTNQSGLERCEGEQDKRLHCYASWRNSSGTIEIVK |
| | QGCWLDDFNCYDRTDCVEKKDSPQVYFCCCEGNMCNEKFSYFPEMEVTQPTS |
| 776 | GAILGRAETRECIYYNANWELERTNQSGLERCEGEQDKRLHCYASWRNSSGTIEIV |
| | KQGCWLDDFNCYDRTDCVATEENPQVYFCCCEGNMCNEKFSYFPEMEVTQPTS |
| 777 | GRGEAETRECIYYNANWELERTNQSGLERCEGEQDKRLHCYASWRNSSGTIEIVK |
| | QGCWLDDFNCYDRTDCVEKKDSPEVYFCCCEGNMCNEKFSYFPEMEVTQPTS |
| 778 | GRGEAETRECIYYNANWELERTNQSGLERCEGEQDKRRHCFATWKNISGSIEIVKQ |
| | GCWLDDFNCYDRTDCVEKKDSPQVYFCCCEGNMCNEKFSYFPEMEVTQPTS |
| 779 | GRGEAETRECIYYNANWELERTNQSGLERCEGEQDKRLHCFATWKNISGSIEIVKQ |
| | GCWLDDFNCYDRTDCVEKKDSPQVYFCCCEGNMCNEKFSYFPEMEVTQPTS |
| 780 | GRGEAETRECIYYNANWELERTNQSGLERCEGEQDKRLHCYASWKNISGSIEIVKQ |
| | GCWLDDFNCYDRTDCVEKKDSPQVYFCCCEGNMCNEKFSYFPEMEVTQPTS |
| 781 | GRGEAETRECIYYNANWELERTNQSGLERCEGEQDKRLHCYASWRNSSGSIEIVK |
| | QGCWLDDFNCYDRTDCVEKKDSPQVYFCCCEGNMCNEKFSYFPEMEVTQPTS |
| 782 | GRGEAETRECIYYNANWELERTNQSGLERCEGEQDKRLHCYASWRNSSGTIELVK |
| | KGCWLDDFNCYDRTDCVEKKDSPQVYFCCCEGNMCNEKFSYFPEMEVTQPTS |
| 783 | GRGEAETRECIYYNANWELERTNQSGLERCEGEQDKRLHCYASWRNSSGTIELVK |
| | KGCWLDDFNCYDRQECVATKDSPQVYFCCCEGNMCNEKFSYFPEMEVTQPTS |
| 784 | GRGEAETRECIYYNANWELERTNQSGLERCEGEQDKRLHCYASWRNSSGTIELVK |
| | KGCWLDDFNCYDRQECVATEENPQVYFCCCEGNMCNEKFSYFPEMEVTQPTS |
| 785 | GRGEAETRECIYYNANWELERTNQSGLERCEGEQDKRLHCYASWRNSSGTIEIVK |
| | QGCWLDDNNCYDRTDCVEKKDSPQVYFCCCEGNMCNEKFSYFPEMEVTQPTS |
| 786 | GRGEAETRECIYYNANWELERTNQSGLERCEGEQDKRLHCYASWRNSSGTIEIVK |
| | QGCWLDDTNCYDRTDCVEKKDSPQVYFCCCEGNMCNEKFSYFPEMEVTQPTS |
| 787 | GRGEAETRECIYYNANWELERTNQSGLERCEGEQDKRLHCYASWRNSSGTIEIVK |
| | QGCWLDDETCYDRTDCVEKKDSPQVYFCCCEGNMCNEKFSYFPEMEVTQPTS |
| 788 | GRGEAETRECIYYNANWELERTNQSGLERCEGEQRKRLHCYASWRNSSGTIEIVK |
| | QGCWLDDFNCYDRTDCVEKKDSPQVYFCCCEGNMCNEKFSYFPEMEVTQPTS |
| 789 | GRGEAETRECIYYNANWELERTNQSGLERCEGEQDKRLHCYASWRNSSGTIEIVK |
| | QGCWLDDFNCYDRTDCVETKDSPQVYFCCCEGNMCNEKFSYFPEMEVTQPTS |
| 790 | GAILGRAETRECIYYNANWELERTNQSGLERCEGEQDKRLHCYASWRNSSGTIEIV |
| | KQGCWLDDFNCYDRTDCVEKKDSPQVYFCCCEGNMCNEKFSYFPEMEVTQPTS |
| 791 | GAILGRSETQECIYYNANWELERTNQSGLERCEGEQDKRLHCYASWRNSSGTIEIV |
| | KQGCWLDDFNCYDRTDCVEKKDSPQVYFCCCEGNMCNEKFSYFPEMEVTQPTS |
| 792 | GRGEAETRECIYYNANWELERTNQSGLERCEGEQDKRLHCYASWRNSSGTIELVK |
| | KGCWLDDFNCYDRQECVATKDSPEVYFCCCEGNMCNEKFSYFPEMEVTQPTS |
| 793 | GRGEAETRECIYYNANWELERTNQSGLERCEGEQDKRLHCYASWRNSSGTIELVK |
| | KGCWLDDFNCYDRQECVATKENPQVYFCCCEGNMCNEKFSYFPEMEVTQPTS |

In some embodiments, the ActRII chimera included in the ActRII chimera ligand trap results from the substitution of one or more amino acid sequence corresponding to a β-sheet and, optionally, one or more intervening sequence (e.g., a sequence between the β-sheets), from one ActRII protein (e.g., ActRIIB) into the corresponding position of the other ActRII protein (e.g., ActRIIA). For example, an ActRII chimera may be produced by replacing one or more amino acid sequence corresponding to a β-sheet and, optionally, one or more or an intervening sequence, in ActRIIB with an amino acid sequence corresponding to the β-sheet and, optionally, the intervening sequence, from ActRIIA. An ActRII chimera may also be produced by replacing one or more amino acid sequence corresponding to a β-sheet and, optionally, one or more intervening sequence, in ActRIIA with an amino acid sequence corresponding to the β-sheet and, optionally, the intervening sequence, from ActRIIB. In the ActRII chimeras, a β-sheet and, optionally, an intervening sequence from one protein is replaced with the corresponding β-sheet and, optionally, the corresponding intervening sequence from the other protein (e.g., the 5^{th} β-sheet from ActRIIA (β_{5A}) can be replaced with the 5^{th} β-sheet from ActRIIB (β_{5B})). Each ActRII protein has seven β-sheets (β₁-β₇) and eight intervening sequences (X₁-X₈). The ActRII chimeras include at least one of β₁ₐ, β₂ₐ, β₃ₐ, β₄ₐ, β₅ₐ, or β₇ₐ and at least one of β_{1b}, β_{2b}, β_{3b}, β_{4b}, β_{5b}, or β_{7b}. Accordingly, an ActRII chimera included in the ActRII chimera ligand trap may have one to five β-sheet substitutions (e.g., 1, 2, 3, 4, or 5 of β₁, β₂, β₃, β₄, β₅, and β₇ from one ActRII protein may be substituted with the corresponding β-sheet sequence from the other ActRII protein). The ActRII chimera may also have one to seven intervening sequence substitutions (e.g., 1, 2, 3, 4, 5, 6, or 7 of X₁, X₂, X₃, X₅, X₆, X₇, and X₈ from one ActRII protein may be substituted with the corresponding intervening sequence from the other ActRII protein). In some embodiments, the β-sheet sequence that is substituted is a minimal β-sheet sequence (e.g., at least HCFATWK (SEQ ID NO: 805), which is a portion of RHCFATWKNI (β₃ₐ) (SEQ ID NO: 804); at least HCYASWR (SEQ ID NO: 807), which is a portion of LHCYASWRNS (β_{3b}) (SEQ ID NO: 806); at least EIVKQGCW (SEQ ID NO: 809), which is a portion of SIEIVKQGCW (β₄ₐ) (SEQ ID NO: 808); at least ELVKKGCW (SEQ ID NO: 811), which is a portion of TIELVKKGCW (β_{4b}) (SEQ ID NO: 810); at least VE, which is a portion of VEK (β₅ₐ); at least V, which is a portion of VAT (β_{5b}); at least SYF, which is a portion of KFSYF (β₇ₐ) (SEQ ID NO: 819); or at least T, which is a portion of RFTHL (β_{7b}) (SEQ ID NO: 820)). The extracellular ActRII chimeras are the same length (e.g., have the same number of amino acids) as wild-type extracellular ActRIIA and ActRIIB, therefore, in embodiments in which minimal β-sheet sequences are substituted, contiguous amino acids from ActRIIA or ActRIIB are used to connect the minimal β-sheet to the neighboring intervening sequences to maintain the length (e.g., the number of amino acids) of the ActRII chimeras (e.g., to prevent the extracellular ActRII chimeras from having fewer amino acids than the corresponding regions of extracellular ActRIIA and ActRIIB). Exemplary ActRII chimera sequences that can be included in an ActRII chimera ligand trap are provided in Table 17. ActRII chimera ligand traps that may be used in the methods described herein include those described in International Patent Application No. PCT/US2022/027399, the disclosure of which is incorporated herein by reference.

TABLE 17
Extracellular ActRII chimera sequences
For the ActRII chimera having the sequence
X₁β₁X₂β₂X₃β₃X₄β₄X₅β₅X₆β₆X₇β₇X₈:

| X₁ | GAILGRSETQ (SEQ ID NO: 794) |
|---|---|
| | or |
| | GRGEAETR (SEQ ID NO: 795) |
| β₁ | ECLFFN (β₁ₐ) (SEQ ID NO: 796) |
| | or |
| | ECIYYN (β_{1b}) (SEQ ID NO: 797) |
| X₂ | ANWEKDRTN (SEQ ID NO: 798) |
| | or |
| | ANWELERTN (SEQ ID NO: 799) |
| β₂ | QTGVEPC (β₂ₐ) (SEQ ID NO: |
| | 800) |
| | or |
| | QSGLERC (β_{2b}) (SEQ ID NO: |
| | 801) |
| X₃ | YGDKDKR (SEQ ID NO: 802) |
| | or |
| | EGEQDKR (SEQ ID NO: 803) |
| β₃ | RHCFATWKNI (β₃ₐ) (SEQ ID |
| | NO: 804) or a portion thereof that |
| | comprises HCFATWK (SEQ ID |
| | NO: 805) |
| | or |
| | LHCYASWRNS (β_{3b}) (SEQ ID |
| | NO: 806) or a portion thereof that |
| | comprises HCYASWR (SEQ ID |
| | NO: 807) |
| X₄ | SG |
| β₄ | SIEIVKQGCW (β₄ₐ) (SEQ ID NO: |
| | 808) or a portion thereof that |
| | comprises EIVKQGCW (SEQ ID |
| | NO: 809) |
| | or |
| | TIELVKKGCW (β_{4b}) (SEQ ID |
| | NO: 810) or a portion thereof that |
| | comprises ELVKKGCW (SEQ ID |
| | NO: 811) |
| X₅ | LDDINCYDRTDC (SEQ ID NO: 812) |
| | or |
| | LDDFNCYDRQEC (SEQ ID NO: 813) |
| β₅ | VEK (β₅ₐ) or a portion thereof that |
| | comprises VE |
| | or |
| | VAT (β_{5b}) or a portion thereof that |
| | comprises V |
| X₆ | KDSPEV (SEQ ID NO: 814) |
| | or |
| | EENPQV (SEQ ID NO: 815) |
| β₆ | YFCCCE (SEQ ID NO: 816) |
| X₇ | GNMCNE (SEQ ID NO: 817) |
| | or |
| | GNFCNE (SEQ ID NO: 818) |
| β₇ | KFSYF (β₇ₐ) (SEQ ID NO: 819) or a |
| | portion thereof that comprises SYF |
| | or |
| | RFTHL (β_{7b}) (SEQ ID NO: 820) or a |
| | portion thereof that comprises T |
| X₈ | PEMEVTQPTS (SEQ ID NO: 821) |
| | or |
| | PEAGGPEVTYEPPPTAPT (SEQ ID NO: |
| | 822) |

In some embodiments, the extracellular ActRII chimeras have an N-terminal truncation of 1-9 amino acids (e.g., 1, 2, 3, 4, 5, 6, 7, 8, or 9 amino acids). The N-terminal truncation can involve the removal of 1-9 amino acids from the N-terminus of any of the chimeras shown in Tables 15-17. The N-terminal truncation can remove amino acids up two to amino acids before the first cysteine (e.g., the two amino acids before the first cysteine (RE or QE) are retained in the N-terminally truncated ActRII chimera ligand traps).

The extracellular ActRII chimera ligand traps may further include a C-terminal extension (e.g., additional amino acids at the C-terminus). The C-terminal extension can add one or more additional amino acids at the C-terminus (e.g., 1, 2, 3, 4, 5, 6 or more additional amino acids) to any of the chimeras shown in Tables 15-17. The C-terminal extension may correspond to sequence from the same position in wild-type ActRIIA or ActRIIB. For example, C-terminal extensions that can be included in the extracellular ActRII chimera ligand traps of the invention are the amino acid sequence NP and the amino acid sequence NPVTPK (SEQ ID NO: 78), which correspond to sequence found in the same position in wild-type ActRIIA.

In some embodiments, an extracellular ActRII chimera ligand trap may further include a moiety (e.g., Fc domain monomer, an Fc domain, an albumin-binding peptide, a fibronectin domain, or a human serum albumin), which may be fused to the N- or C-terminus (e.g., C-terminus) of the extracellular ActRII chimera by way of a linker or other covalent bonds. An ActRII chimera ligand trap including an extracellular ActRII chimera fused to an Fc domain monomer may form a dimer (e.g., homodimer or heterodimer) through the interaction between two Fc domain monomers, which combine to form an Fc domain in the dimer.

### Fc Domains

In some embodiments, an ActRII ligand trap described herein may include an extracellular portion of ActRIIA, ActRIIB, a variant thereof, or a chimera thereof fused to an Fc domain monomer of an immunoglobulin or a fragment of an Fc domain to increase the serum half-life of the polypeptide. An ActRII ligand trap including an extracellular portion of ActRIIA, ActRIIB, a variant thereof, or a chimera thereof fused to an Fc domain monomer may form a dimer (e.g., homodimer or heterodimer) through the interaction between two Fc domain monomers, which form an Fc domain in the dimer. As conventionally known in the art, an Fc domain is the protein structure that is found at the C-terminus of an immunoglobulin. An Fc domain includes two Fc domain monomers that are dimerized by the interaction between the C_{H}3 antibody constant domains. An Fc domain forms the minimum structure that binds to an Fc receptor, e.g., FcγRI, FcγRIIa, FcγIIb, FcγIIIa, FcγIIIb, FcγRIV. In some embodiments, an Fc domain may be mutated to lack effector functions, typical of a “dead” Fc domain. For example, an Fc domain may include specific amino acid substitutions that are known to minimize the interaction between the Fc domain and an Fcγ receptor. In some embodiments, an Fc domain is from an IgG1 antibody and includes amino acid substitutions L234A, L235A, and G237A. In some embodiments, an Fc domain is from an IgG1 antibody and includes amino acid substitutions D265A, K322A, and N434A. The aforementioned amino acid positions are defined according to Kabat (Sequences of Proteins of Immunological Interest, 5th Ed. Public Health Service, National Institutes of Health, Bethesda, MD. (1991)). The Kabat numbering of amino acid residues may be determined for a given antibody by alignment at regions of homology of the sequence of the antibody with a “standard” Kabat numbered sequence. Furthermore, in some embodiments, an Fc domain does not induce any immune system-related response. For example, the Fc domain in a dimer of an ActRII ligand trap including an extracellular portion of ActRIIA, ActRIIB, a variant thereof, or a chimera thereof fused to an Fc domain monomer may be modified to reduce the interaction or binding between the Fc domain and an Fcγ receptor. The sequence of an Fc domain monomer that may be fused to an extracellular portion of ActRIIA, ActRIIB, a variant thereof, or a chimera thereof variant is shown below (SEQ ID NO: 97):

THTCPPCPAPELLGGPSVFLFPPKPKDTLMISRTPEVTCVVVDVSHEDPE
VKFNWYVDGVEVHNAKTKPREEQYNSTYRVVSVLTVLHQDWLNGKEYKCK
VSNKALPVPIEKTISKAKGQPREPQVYTLPPSREEMTKNQVSLTCLVKGF
YPSDIAVEWESNGQPENNYKTTPPVLDSDGPFFLYSKLTVDKSRWQQGNV
FSCSVMHEALHNHYTQKSLSLSPGK

In some embodiments, an Fc domain is from an IgG1 antibody and includes amino acid substitutions L12A, L13A, and G15A, relative to the sequence of SEQ ID NO: 97. In some embodiments, an Fc domain is from an IgG1 antibody and includes amino acid substitutions D43A, K100A, and N212A, relative to the sequence of SEQ ID NO: 97. In some embodiments, the terminal lysine is absent from the Fc domain monomer having the sequence of SEQ ID NO: 97. In some embodiments, an extracellular portion of ActRIIA, ActRIIB, a variant thereof, or a chimera thereof described herein (e.g., an extracellular ActRIIA variant having the sequence of any one of SEQ ID NOs: 1-72 (e.g., SEQ ID NOs: 6-72)) may be fused to the N- or C-terminus of an Fc domain monomer (e.g., SEQ ID NO: 97) through conventional genetic or chemical means, e.g., chemical conjugation. If desired, a linker (e.g., a spacer) can be inserted between the extracellular portion of ActRIIA, ActRIIB, a variant thereof, or a chimera thereof and the Fc domain monomer. The Fc domain monomer can be fused to the N- or C-terminus (e.g., C-terminus) of the extracellular portion of ActRIIA, ActRIIB, a variant thereof, or a chimera thereof.

In some embodiments, an ActRII ligand trap described herein may include an extracellular portion of ActRIIA, ActRIIB, a variant thereof, or a chimera thereof fused to an Fc domain. In some embodiments, the Fc domain contains one or more amino acid substitutions that reduce or inhibit Fc domain dimerization. In some embodiments, the Fc domain contains a hinge domain. The Fc domain can be of immunoglobulin antibody isotype IgG, IgE, IgM, IgA, or IgD. Additionally, the Fc domain can be an IgG subtype (e.g., IgG1, IgG2a, IgG2b, IgG3, or IgG4). The Fc domain can also be a non-naturally occurring Fc domain, e.g., a recombinant Fc domain.

Methods of engineering Fc domains that have reduced dimerization are known in the art. In some embodiments, one or more amino acids with large side-chains (e.g., tyrosine or tryptophan) may be introduced to the C_{H}3-C_{H}3 dimer interface to hinder dimer formation due to steric clash. In other embodiments, one or more amino acids with small side-chains (e.g., alanine, valine, or threonine) may be introduced to the C_{H}3-C_{H}3 dimer interface to remove favorable interactions. Methods of introducing amino acids with large or small side-chains in the C_{H}3 domain are described in, e.g., Ying et al. (J Biol Chem. 287:19399-19408, 2012), U.S. Patent Publication No. 2006/0074225, U.S. Pat. Nos. 8,216,805 and 5,731,168, Ridgway et al. (Protein Eng. 9:617-612, 1996), Atwell et al. (J Mol Biol. 270:26-35, 1997), and Merchant et al. (Nat Biotechnol. 16:677-681, 1998), all of which are incorporated herein by reference in their entireties.

In yet other embodiments, one or more amino acid residues in the C_{H}3 domain that make up the C_{H}3-C_{H}3 interface between two Fc domains are replaced with positively charged amino acid residues (e.g., lysine, arginine, or histidine) or negatively charged amino acid residues (e.g., aspartic acid or glutamic acid) such that the interaction becomes electrostatically unfavorable depending on the specific charged amino acids introduced. Methods of introducing charged amino acids in the C_{H}3 domain to disfavor or prevent dimer formation are described in, e.g., Ying et al. (J Biol Chem. 287:19399-19408, 2012), U.S. Patent Publication Nos. 2006/0074225, 2012/0244578, and 2014/0024111, all of which are incorporated herein by reference in their entireties.

In some embodiments of the invention, an Fc domain includes one or more of the following amino acid substitutions: T366W, T366Y, T394W, F405W, Y349T, Y349E, Y349V, L351T, L351H, L351N, L352K, P353S, S354D, D356K, D356R, D356S, E357K, E357R, E357Q, S364A, T366E, L368T, L368Y, L368E, K370E, K370D, K370Q, K392E, K392D, T394N, P395N, P396T, V397T, V397Q, L398T, D399K, D399R, D399N, F405T, F405H, F405R, Y407T, Y407H, Y407I, K409E, K409D, K409T, and K4091, relative to the sequence of human IgG1. In some embodiments, the terminal lysine is absent from the Fc domain amino acid sequence. In one particular embodiment, an Fc domain includes the amino acid substitution T366W, relative to the sequence of human IgG1. The sequence of an Fc domain (a wild-type Fc domain) is shown below in SEQ ID NO: 84:

DKTHTCPPCPAPELLGGPSVFLFPPKPKDTLMISRTPEVTCVVVDVSHED
PEVKFNWYVDGVEVHNAKTKPREEQYNSTYRVVSVLTVLHQDWLNGKEYK
CKVSNKALPAPIEKTISKAKGQPREPQVYTLPPSRDELTKNQVSLTCLVK
GFYPSDIAVEWESNGQPENNYKTTPPVLDSDGSFFLYSKLTVDKSRWQQG
NVFSCSVMHEALHNHYTQKSLSLSPGK.

An exemplary sequence for a wild-type Fc domain lacking the terminal lysine is provided below (SEQ ID NO: 79):

DKTHTCPPCPAPELLGGPSVFLFPPKPKDTLMISRTPEVTCVVVDVSHED
PEVKFNWYVDGVEVHNAKTKPREEQYNSTYRVVSVLTVLHQDWLNGKEYK
CKVSNKALPAPIEKTISKAKGQPREPQVYTLPPSRDELTKNQVSLTCLVK
GFYPSDIAVEWESNGQPENNYKTTPPVLDSDGSFFLYSKLTVDKSRWQQG
NVFSCSVMHEALHNHYTQKSLSLSPG.

### Albumin-Binding Peptide

In some embodiments, an ActRII ligand trap described herein may include an extracellular portion of ActRIIA, ActRIIB, a variant thereof, or a chimera thereof fused to a serum protein-binding peptide. Binding to serum protein peptides can improve the pharmacokinetics of protein pharmaceuticals.

As one example, albumin-binding peptides that can be used in the methods and compositions described here are generally known in the art. In one embodiment, the albumin binding peptide includes the sequence DICLPRWGCLW (SEQ ID NO: 83).

In the present invention, albumin-binding peptides may be joined to the N- or C-terminus (e.g., C-terminus) of an extracellular portion of ActRIIA, ActRIIB, a variant thereof, or a chimera thereof described herein (e.g., an extracellular ActRIIA variant having the sequence of any one of SEQ ID NOs: 1-72 (e.g., SEQ ID NOs: 6-72)) to increase the serum half-life of the extracellular ActRIIA variant. In some embodiments, an albumin-binding peptide is joined, either directly or through a linker, to the N- or C-terminus of an extracellular portion of ActRIIA, ActRIIB, a variant thereof, or a chimera thereof.

In some embodiments, an extracellular portion of ActRIIA, ActRIIB, a variant thereof, or a chimera thereof described herein (e.g., an extracellular ActRIIA variant having the sequence of any one of SEQ ID NOs: 1-72 (e.g., SEQ ID NOs: 6-72)) may be fused to the N- or C-terminus of albumin-binding peptide (e.g., SEQ ID NO: 83) through conventional genetic or chemical means, e.g., chemical conjugation. If desired, a linker (e.g., a spacer) can be inserted between the extracellular portion of ActRIIA, ActRIIB, a variant thereof, or a chimera thereof and the albumin-binding peptide. Without being bound to a theory, it is expected that inclusion of an albumin-binding peptide in an extracellular portion of ActRIIA, ActRIIB, a variant thereof, or a chimera thereof described herein may lead to prolonged retention of the therapeutic protein through its binding to serum albumin.

### Fibronectin Domain

In some embodiments, an ActRII ligand trap described herein may include an extracellular portion of ActRIIA, ActRIIB, a variant thereof, or a chimera thereof fused to fibronectin domains. Binding to fibronectin domains can improve the pharmacokinetics of protein pharmaceuticals.

Fibronectin domain is a high molecular weight glycoprotein of the extracellular matrix, or a fragment thereof, that binds to, e.g., membrane-spanning receptor proteins such as integrins and extracellular matrix components such as collagens and fibrins. In some embodiments of the present invention, a fibronectin domain is joined to the N- or C-terminus (e.g., C-terminus) of an extracellular portion of ActRIIA, ActRIIB, a variant thereof, or a chimera thereof described herein (e.g., an extracellular ActRIIA variant having the sequence of any one of SEQ ID NOs: 1-72 (e.g., SEQ ID NOs: 6-72)) to increase the serum half-life of the extracellular portion of ActRIIA, ActRIIB, a variant thereof, or a chimera thereof. A fibronectin domain can be joined, either directly or through a linker, to the N- or C-terminus of an extracellular portion of ActRIIA, ActRIIB, a variant thereof, or a chimera thereof.

As one example, fibronectin domains that can be used in the methods and compositions described here are generally known in the art. In one embodiment, the fibronectin domain is a fibronectin type III domain (SEQ ID NO: 82, below) having amino acids 610-702 of the sequence of UniProt ID NO: P02751.

GPVEVFITETPSQPNSHPIQWNAPQPSHISKYILRWRPKNSVGRWKEATI
PGHLNSYTIKGLKPGVVYEGQLISIQQYGHQEVTRFDFTTTST

In another embodiment, the fibronectin domain is an adnectin protein.

In some embodiments, an extracellular portion of ActRIIA, ActRIIB, a variant thereof, or a chimera thereof described herein (e.g., an extracellular ActRIIA variant having the sequence of any one of SEQ ID NOs: 1-72 (e.g., SEQ ID NOs: 6-72)) may be fused to the N- or C-terminus of a fibronectin domain (e.g., SEQ ID NO: 82) through conventional genetic or chemical means, e.g., chemical conjugation. If desired, a linker (e.g., a spacer) can be inserted between the extracellular portion of ActRIIA, ActRIIB, a variant thereof, or a chimera thereof and the fibronectin domain. Without being bound to a theory, it is expected that inclusion of a fibronectin domain in an extracellular portion of ActRIIA, ActRIIB, a variant thereof, or a chimera thereof described herein may lead to prolonged retention of the therapeutic protein through its binding to integrins and extracellular matrix components such as collagens and fibrins.

### Serum Albumin

In some embodiments, an ActRII ligand trap described herein may include an extracellular portion of ActRIIA, ActRIIB, a variant thereof, or a chimera thereof fused to serum albumin. Binding to serum albumins can improve the pharmacokinetics of protein pharmaceuticals.

Serum albumin is a globular protein that is the most abundant blood protein in mammals. Serum albumin is produced in the liver and constitutes about half of the blood serum proteins. It is monomeric and soluble in the blood. Some of the most crucial functions of serum albumin include transporting hormones, fatty acids, and other proteins in the body, buffering pH, and maintaining osmotic pressure needed for proper distribution of bodily fluids between blood vessels and body tissues. In preferred embodiments, serum albumin is human serum albumin. In some embodiments of the present invention, a human serum albumin is joined to the N- or C-terminus (e.g., C-terminus) of an extracellular portion of ActRIIA, ActRIIB, a variant thereof, or a chimera thereof described herein (e.g., an extracellular ActRIIA variant having the sequence of any one of SEQ ID NOs: 1-72 (e.g., SEQ ID NOs: 6-72)) to increase the serum half-life of the extracellular portion of ActRIIA, ActRIIB, a variant thereof, or a chimera thereof. A human serum albumin can be joined, either directly or through a linker, to the N- or C-terminus of an extracellular portion of ActRIIA, ActRIIB, a variant thereof, or a chimera thereof.

As one example, serum albumins that can be used in the methods and compositions described herein are generally known in the art. In one embodiment, the serum albumin includes the sequence of UniProt ID NO: P02768 (SEQ ID NO: 81, below).

MKWVTFISLLFLFSSAYSRGVFRRDAHKSEVAHRFKDLGEENFKA
LVLIAFAQYLQQCPFEDHVKLVNEVTEFAKTCVADESAENCDKSL
HTLFGDKLCTVATLRETYGEMADCCAKQEPERNECFLQHKDDNPN
LPRLVRPEVDVMCTAFHDNEETFLKKYLYEIARRHPYFYAPELLF
FAKRYKAAFTECCQAADKAACLLPKLDELRDEGKASSAKQRLKCA
SLQKFGERAFKAWAVARLSQRFPKAEFAEVSKLVTDLTKVHTECC
HGDLLECADDRADLAKYICENQDSISSKLKECCEKPLLEKSHCIA
EVENDEMPADLPSLAADFVESKDVCKNYAEAKDVFLGMFLYEYAR
RHPDYSVVLLLRLAKTYETTLEKCCAAADPHECYAKVFDEFKPLV
EEPQNLIKQNCELFEQLGEYKFQNALLVRYTKKVPQVSTPTLVEV
SRNLGKVGSKCCKHPEAKRMPCAEDYLSVVLNQLCVLHEKTPVSD
RVTKCCTESLVNRRPCFSALEVDETYVPKEFNAETFTFHADICTL
SEKERQIKKQTALVELVKHKPKATKEQLKAVMDDFAAFVEKCCKA
DDKETCFAEEGKKLVAASQAALGL

In some embodiments, an extracellular portion of ActRIIA, ActRIIB, a variant thereof, or a chimera thereof described herein (e.g., an extracellular ActRIIA variant having the sequence of any one of SEQ ID NOs: 1-72 (e.g., SEQ ID NOs: 6-72)) may be fused to the N- or C-terminus of a human serum albumin (e.g., SEQ ID NO: 81) through conventional genetic or chemical means, e.g., chemical conjugation. If desired, a linker (e.g., a spacer) can be inserted between the extracellular portion of ActRIIA, ActRIIB, a variant thereof, or a chimera thereof and the human serum albumin. Without being bound to a theory, it is expected that inclusion of a human serum albumin in an extracellular portion of ActRIIA, ActRIIB, a variant thereof, or a chimera thereof described herein may lead to prolonged retention of the therapeutic protein.

### Linkers

An ActRII ligand trap described herein may include an extracellular portion of ActRIIA, ActRIIB, a variant thereof, or a chimera thereof (e.g., an extracellular ActRIIA variant having a sequence of any one of SEQ ID NOs: 1-72 (e.g., SEQ ID NOs: 6-72)) fused to a moiety by way of a linker. In some embodiments, the moiety increases stability of the polypeptide. Exemplary moieties include an Fc domain monomer, an Fc domain, an albumin-binding peptide, a fibronectin domain, or a human serum albumin. In the present invention, a linker between a moiety (e.g., an Fc domain monomer (e.g., the sequence of SEQ ID NO: 97), an Fc domain (e.g., SEQ ID NO: 84 or SEQ ID NO: 79), an albumin-binding peptide (e.g., SEQ ID NO: 83), a fibronectin domain (e.g., SEQ ID NO: 82), or a human serum albumin (e.g., SEQ ID NO: 81)) and an extracellular portion of ActRIIA, ActRIIB, a variant thereof, or a chimera thereof (e.g., an extracellular ActRIIA variant having the sequence of any one of SEQ ID NOs: 1-72 (e.g., SEQ ID NOs: 6-72)), can be an amino acid spacer including 1-200 amino acids. Suitable peptide spacers are known in the art, and include, for example, peptide linkers containing flexible amino acid residues such as glycine, alanine, and serine. In some embodiments, a spacer can contain motifs, e.g., multiple or repeating motifs, of GA, GS, GG, GGA, GGS, GGG, GGGA (SEQ ID NO: 98), GGGS (SEQ ID NO: 99), GGGG (SEQ ID NO: 100), GGGGA (SEQ ID NO: 101), GGGGS (SEQ ID NO: 102), GGGGG (SEQ ID NO: 103), GGAG (SEQ ID NO: 104), GGSG (SEQ ID NO: 105), AGGG (SEQ ID NO: 106), or SGGG (SEQ ID NO: 107). In some embodiments, a spacer can contain 2 to 12 amino acids including motifs of GA or GS, e.g., GA, GS, GAGA (SEQ ID NO: 108), GSGS (SEQ ID NO: 109), GAGAGA (SEQ ID NO: 110), GSGSGS (SEQ ID NO: 111), GAGAGAGA (SEQ ID NO: 112), GSGSGSGS (SEQ ID NO: 113), GAGAGAGAGA (SEQ ID NO: 114), GSGSGSGSGS (SEQ ID NO: 115), GAGAGAGAGAGA (SEQ ID NO: 116), and GSGSGSGSGSGS (SEQ ID NO: 117). In some embodiments, a spacer can contain 3 to 12 amino acids including motifs of GGA or GGS, e.g., GGA, GGS, GGAGGA (SEQ ID NO: 118), GGSGGS (SEQ ID NO: 119), GGAGGAGGA (SEQ ID NO: 120), GGSGGSGGS (SEQ ID NO: 121), GGAGGAGGAGGA (SEQ ID NO: 122), and GGSGGSGGSGGS (SEQ ID NO: 123). In yet some embodiments, a spacer can contain 4 to 12 amino acids including motifs of GGAG (SEQ ID NO: 104), GGSG (SEQ ID NO: 105), e.g., GGAG (SEQ ID NO: 104), GGSG (SEQ ID NO: 105), GGAGGGAG (SEQ ID NO: 124), GGSGGGSG (SEQ ID NO: 125), GGAGGGAGGGAG (SEQ ID NO: 126), and GGSGGGSGGGSG (SEQ ID NO: 127). In some embodiments, a spacer can contain motifs of GGGGA (SEQ ID NO: 101) or GGGGS (SEQ ID NO: 102), e.g., GGGGAGGGGAGGGGA (SEQ ID NO: 128) and GGGGSGGGGSGGGGS (SEQ ID NO: 129). In some embodiments of the invention, an amino acid spacer between a moiety (e.g., an Fc domain monomer, an Fc domain, an albumin-binding peptide, a fibronectin domain, or a human serum albumin) and an extracellular portion of ActRIIA, ActRIIB, a variant thereof, or a chimera thereof (e.g., an extracellular ActRIIA variant having the sequence of any one of SEQ ID NOs: 1-72 (e.g., SEQ ID NOs: 6-72)) may be GGG, GGGA (SEQ ID NO: 98), GGGG (SEQ ID NO: 100), GGGAG (SEQ ID NO: 130), GGGAGG (SEQ ID NO: 131), or GGGAGGG (SEQ ID NO: 132).

In some embodiments, a spacer can also contain amino acids other than glycine, alanine, and serine, e.g., AAAL (SEQ ID NO: 133), AAAK (SEQ ID NO: 134), AAAR (SEQ ID NO: 135), EGKSSGSGSESKST (SEQ ID NO: 136), GSAGSAAGSGEF (SEQ ID NO: 137), AEAAAKEAAAKA (SEQ ID NO: 96), KESGSVSSEQLAQFRSLD (SEQ ID NO: 95), GENLYFQSGG (SEQ ID NO: 94), SACYCELS (SEQ ID NO: 93), RSIAT (SEQ ID NO: 92), RPACKIPNDLKQKVMNH (SEQ ID NO: 91), GGSAGGSGSGSSGGSSGASGTGTAGGTGSGSGTGSG (SEQ ID NO: 90), AAANSSIDLISVPVDSR (SEQ ID NO: 89), or GGSGGGSEGGGSEGGGSEGGGSEGGGSEGGGSGGGS (SEQ ID NO: 88). In some embodiments, a spacer can contain motifs, e.g., multiple or repeating motifs, of EAAAK (SEQ ID NO: 87). In some embodiments, a spacer can contain motifs, e.g., multiple or repeating motifs, of proline-rich sequences such as (XP)ₙ, in which X may be any amino acid (e.g., A, K, or E) and n is from 1-5, and PAPAP (SEQ ID NO: 86).

The length of the peptide spacer and the amino acids used can be adjusted depending on the two proteins involved and the degree of flexibility desired in the final protein fusion polypeptide. The length of the spacer can be adjusted to ensure proper protein folding and avoid aggregate formation.

In some embodiments, the linker between a moiety (e.g., an Fc domain monomer (e.g., the sequence of SEQ ID NO: 97), an Fc domain (e.g., SEQ ID NO: 84 or SEQ ID NO: 79), an albumin-binding peptide (e.g., SEQ ID NO: 83), a fibronectin domain (e.g., SEQ ID NO: 82), or a human serum albumin (e.g., SEQ ID NO: 81)) and an extracellular portion of ActRIIA, ActRIIB, a variant thereof, or a chimera thereof described herein (e.g., an extracellular ActRIIA variant having the sequence of any one of SEQ ID NOs: 1-72 (e.g., SEQ ID NOs: 6-72), is an amino acid spacer having the sequence GGG. For example, an ActRIIA ligand trap of the invention can contain an extracellular ActRIIA variant (e.g., any one of SEQ ID NOs: 6-72) fused to an Fc domain (e.g., SEQ ID NO: 79) by a GGG linker. An exemplary polypeptide containing an ActRIIA variant of SEQ ID NO: 69, a GGG linker, and an Fc domain lacking a terminal lysine (SEQ ID NO: 79) is provided below (SEQ ID NO: 80):

GAILGRSETQECLFYNANWELERTNQTGVERCEGEKDKRLHCYAT
WRNISGSIEIVKKGCWLDDFNCYDRTDCVETEENPQVYFCCCEGN
MCNEKFSYFPEMEVTQPTSGGGDKTHTCPPCPAPELLGGPSVFLF
PPKPKDTLMISRTPEVTCVVVDVSHEDPEVKFNWYVDGVEVHNAK
TKPREEQYNSTYRVVSVLTVLHQDWLNGKEYKCKVSNKALPAPIE
KTISKAKGQPREPQVYTLPPSRDELTKNQVSLTCLVKGFYPSDIA
VEWESNGQPENNYKTTPPVLDSDGSFFLYSKLTVDKSRWQQGNVF
SCSVMHEALHNHYTQKSLSLSPG

### Vectors, Host Cells, and Protein Production

The ActRII signaling inhibitors of the invention can be produced from a host cell. A host cell refers to a vehicle that includes the necessary cellular components, e.g., organelles, needed to express the polypeptides and fusion polypeptides described herein from their corresponding nucleic acids. The nucleic acids may be included in nucleic acid vectors that can be introduced into the host cell by conventional techniques known in the art (e.g., transformation, transfection, electroporation, calcium phosphate precipitation, direct microinjection, infection, or the like). The choice of nucleic acid vectors depends in part on the host cells to be used. Generally, preferred host cells are of either eukaryotic (e.g., mammalian) or prokaryotic (e.g., bacterial) origin.

### Nucleic Acid Vector Construction and Host Cells

A nucleic acid sequence encoding the amino acid sequence of a polypeptide of the invention (i.e., an ActRII signaling inhibitor) may be prepared by a variety of methods known in the art. These methods include, but are not limited to, oligonucleotide-mediated (or site-directed) mutagenesis and PCR mutagenesis. A nucleic acid molecule encoding a polypeptide of the invention may be obtained using standard techniques, e.g., gene synthesis. Alternatively, for the production of ActRII ligand traps, a nucleic acid molecule encoding a wild-type portion of extracellular ActRIIA or ActRIIB may be mutated to include specific amino acid substitutions using standard techniques in the art, e.g., QuikChange™ mutagenesis. Nucleic acid molecules can be synthesized using a nucleotide synthesizer or PCR techniques.

A nucleic acid sequence encoding a polypeptide of the invention may be inserted into a vector capable of replicating and expressing the nucleic acid molecule in prokaryotic or eukaryotic host cells. Many vectors are available in the art and can be used for the purpose of the invention. Each vector may include various components that may be adjusted and optimized for compatibility with the particular host cell. For example, the vector components may include, but are not limited to, an origin of replication, a selection marker gene, a promoter, a ribosome binding site, a signal sequence, the nucleic acid sequence encoding protein of interest, and a transcription termination sequence.

In some embodiments, mammalian cells may be used as host cells for the invention. Examples of mammalian cell types include, but are not limited to, human embryonic kidney (HEK) (e.g., HEK293, HEK 293F), Chinese hamster ovary (CHO), HeLa, COS, PC3, Vero, MC3T3, NS0, Sp2/0, VERY, BHK, MDCK, W138, BT483, Hs578T, HTB2, BT20, T47D, NS0 (a murine myeloma cell line that does not endogenously produce any immunoglobulin chains), CRL7O3O, and HsS78Bst cells. In some embodiments, E. coli cells may also be used as host cells for the invention. Examples of E. coli strains include, but are not limited to, E. coli 294 (ATCC® 31,446), E. coli λ 1776 (ATCC® 31,537, E. coli BL21 (DE3) (ATCC® BAA-1025), and E. coli RV308 (ATCC®31,608). Different host cells have characteristic and specific mechanisms for the posttranslational processing and modification of protein products (e.g., glycosylation). Appropriate cell lines or host systems may be chosen to ensure the correct modification and processing of the polypeptide expressed. The above-described expression vectors may be introduced into appropriate host cells using conventional techniques in the art, e.g., transformation, transfection, electroporation, calcium phosphate precipitation, and direct microinjection. Once the vectors are introduced into host cells for protein production, host cells are cultured in conventional nutrient media modified as appropriate for inducing promoters, selecting transformants, or amplifying the genes encoding the desired sequences. Methods for expression of therapeutic proteins are known in the art, see, for example, Paulina Balbas, Argelia Lorence (eds.) Recombinant Gene Expression: Reviews and Protocols (Methods in Molecular Biology), Humana Press; 2nd ed. 2004 and Vladimir Voynov and Justin A. Caravella (eds.) Therapeutic Proteins: Methods and Protocols (Methods in Molecular Biology) Humana Press; 2nd ed. 2012.

### Protein Production, Recovery, and Purification

Host cells used to produce the polypeptides of the invention may be grown in media known in the art and suitable for culturing of the selected host cells. Examples of suitable media for mammalian host cells include Minimal Essential Medium (MEM), Dulbecco's Modified Eagle's Medium (DMEM), Expi293™ Expression Medium, DMEM with supplemented fetal bovine serum (FBS), and RPMI-1640. Examples of suitable media for bacterial host cells include Luria broth (LB) plus necessary supplements, such as a selection agent, e.g., ampicillin. Host cells are cultured at suitable temperatures, such as from about 20 ºC to about 39° C., e.g., from 25° C. to about 37° C., preferably 37° C., and CO₂ levels, such as 5 to 10%. The pH of the medium is generally from about 6.8 to 7.4, e.g., 7.0, depending mainly on the host organism. If an inducible promoter is used in the expression vector of the invention, protein expression is induced under conditions suitable for the activation of the promoter.

In some embodiments, depending on the expression vector and the host cells used, the expressed protein may be secreted from the host cells (e.g., mammalian host cells) into the cell culture media. Protein recovery may involve filtering the cell culture media to remove cell debris. The proteins may be further purified. A polypeptide of the invention may be purified by any method known in the art of protein purification, for example, by chromatography (e.g., ion exchange, affinity, and size-exclusion column chromatography), centrifugation, differential solubility, or by any other standard technique for the purification of proteins. For example, the protein can be isolated and purified by appropriately selecting and combining affinity columns such as Protein A column (e.g., POROS Protein A chromatography) with chromatography columns (e.g., POROS HS-50 cation exchange chromatography), filtration, ultra-filtration, salting-out and dialysis procedures.

In other embodiments, host cells may be disrupted, e.g., by osmotic shock, sonication, or lysis, to recover the expressed protein. Once the cells are disrupted, cell debris may be removed by centrifugation or filtration. In some instances, a polypeptide can be conjugated to marker sequences, such as a peptide to facilitate purification. An example of a marker amino acid sequence is a hexa-histidine peptide (His-tag), which binds to nickel-functionalized agarose affinity column with micromolar affinity. Other peptide tags useful for purification include, but are not limited to, the hemagglutinin “HA” tag, which corresponds to an epitope derived from influenza hemagglutinin protein (Wilson et al., Cell 37:767, 1984).

Alternatively, the polypeptides of the invention can be produced by the cells of a subject (e.g., a human), e.g., in the context of gene therapy, by administrating a vector (such as a viral vector (e.g., a retroviral vector, adenoviral vector, poxviral vector (e.g., vaccinia viral vector, such as Modified Vaccinia Ankara (MVA)), adeno-associated viral vector, and alphaviral vector)) containing a nucleic acid molecule encoding the polypeptide of the invention. The vector, once inside a cell of the subject (e.g., by transformation, transfection, electroporation, calcium phosphate precipitation, direct microinjection, infection, etc.) will promote expression of the polypeptide, which is then secreted from the cell. If treatment of a disease or disorder is the desired outcome, no further action may be required. If collection of the protein is desired, blood may be collected from the subject and the protein purified from the blood by methods known in the art.

### Pharmaceutical Compositions and Preparations

The invention features pharmaceutical compositions that include the polypeptides described herein (e.g., an ActRII signaling inhibitor, such as an ActRII ligand trap including an extracellular portion of ActRIIA, ActRIIB, a variant thereof, or a chimera thereof (e.g., an extracellular ActRIIA variant having the sequence of any one of SEQ ID NOs: 1-72 (e.g., SEQ ID NOs: 6-72)). In some embodiments, a pharmaceutical composition of the invention includes an ActRIIA ligand trap including an extracellular ActRIIA variant (e.g., an extracellular ActRIIA variant having the sequence of any one of SEQ ID NOs: 1-70 (e.g., SEQ ID NOs: 6-70)) with a C-terminal extension (e.g., 1, 2, 3, 4, 5, 6 or more additional amino acids) as the therapeutic protein. In some embodiments, a pharmaceutical composition of the invention includes an ActRII ligand trap including an extracellular portion of ActRIIA, ActRIIB, a variant thereof, or a chimera thereof (e.g., an extracellular ActRIIA variant having the sequence of any one of SEQ ID NOs: 1-72 (e.g., SEQ ID NOs: 6-72)) fused to a moiety (e.g., Fc domain monomer, or a dimer thereof, an Fc domain, an albumin-binding peptide, a fibronectin domain, or a human serum albumin) as the therapeutic protein. In some embodiments, a pharmaceutical composition of the invention including a polypeptide of the invention may be used in combination with other agents (e.g., therapeutic biologics and/or small molecules) or compositions in a therapy. In addition to a therapeutically effective amount of the polypeptide, the pharmaceutical composition may include one or more pharmaceutically acceptable carriers or excipients, which can be formulated by methods known to those skilled in the art. In some embodiments, a pharmaceutical composition of the invention includes a nucleic acid molecule (DNA or RNA, e.g., mRNA) encoding a polypeptide of the invention, or a vector containing such a nucleic acid molecule.

Acceptable carriers and excipients in the pharmaceutical compositions are nontoxic to recipients at the dosages and concentrations employed. Acceptable carriers and excipients may include buffers such as phosphate, citrate, HEPES, and TAE, antioxidants such as ascorbic acid and methionine, preservatives such as hexamethonium chloride, octadecyldimethylbenzyl ammonium chloride, resorcinol, and benzalkonium chloride, proteins such as human serum albumin, gelatin, dextran, and immunoglobulins, hydrophilic polymers such as polyvinylpyrrolidone, amino acids such as glycine, glutamine, histidine, arginine, and lysine, and carbohydrates such as glucose, mannose, sucrose, and sorbitol. Pharmaceutical compositions of the invention can be administered parenterally in the form of an injectable formulation. Pharmaceutical compositions for injection can be formulated using a sterile solution or any pharmaceutically acceptable liquid as a vehicle. Pharmaceutically acceptable vehicles include, but are not limited to, sterile water, physiological saline, and cell culture media (e.g., Dulbecco's Modified Eagle Medium (DMEM), α-Modified Eagles Medium (α-MEM), F-12 medium). Formulation methods are known in the art, see e.g., Banga (ed.) Therapeutic Peptides and Proteins: Formulation, Processing and Delivery Systems (3rd ed.) Taylor & Francis Group, CRC Press (2015).

The pharmaceutical compositions of the invention may be prepared in microcapsules, such as hydroxylmethylcellulose or gelatin-microcapsule and poly-(methylmethacrylate) microcapsule. The pharmaceutical compositions of the invention may also be prepared in other drug delivery systems such as liposomes, albumin microspheres, microemulsions, nanoparticles, and nanocapsules. Such techniques are described in Remington: The Science and Practice of Pharmacy 22^{nd} edition (2012). The pharmaceutical compositions to be used for in vivo administration must be sterile. This is readily accomplished by filtration through sterile filtration membranes.

The pharmaceutical compositions of the invention may also be prepared as a sustained-release formulation. Suitable examples of sustained-release preparations include semipermeable matrices of solid hydrophobic polymers containing the polypeptides of the invention. Examples of sustained release matrices include polyesters, hydrogels, polylactides, copolymers of L-glutamic acid and γ ethyl-L-glutamate, non-degradable ethylene-vinyl acetate, degradable lactic acid-glycolic acid copolymers such as LUPRON DEPOT™, and poly-D-(−)-3-hydroxybutyric acid. Some sustained-release formulations enable release of molecules over a few months, e.g., one to six months, while other formulations release pharmaceutical compositions of the invention for shorter time periods, e.g., days to weeks.

The pharmaceutical composition may be formed in a unit dose form as needed. The amount of active component, e.g., a polypeptide of the invention, included in the pharmaceutical preparations is such that a suitable dose within the designated range is provided (e.g., a dose within the range of 0.01-100 mg/kg of body weight).

The pharmaceutical composition for gene therapy can be in an acceptable diluent or can include a slow-release matrix in which the gene delivery vehicle is imbedded. If hydrodynamic injection is used as the delivery method, the pharmaceutical composition containing a nucleic acid molecule encoding a polypeptide described herein or a vector (e.g., a viral vector) containing the nucleic acid molecule is delivered rapidly in a large fluid volume intravenously. Vectors that may be used as in vivo gene delivery vehicle include, but are not limited to, retroviral vectors, adenoviral vectors, poxviral vectors (e.g., vaccinia viral vectors, such as Modified Vaccinia Ankara), adeno-associated viral vectors, and alphaviral vectors.

### Routes, Dosage, and Administration

Pharmaceutical compositions that include the polypeptides of the invention as the therapeutic proteins may be formulated for, e.g., intravenous administration, parenteral administration, subcutaneous administration, intramuscular administration, intra-arterial administration, intrathecal administration, or intraperitoneal administration. The pharmaceutical composition may also be formulated for, or administered via, oral, nasal, spray, aerosol, rectal, or vaginal administration. For injectable formulations, various effective pharmaceutical carriers are known in the art. See, e.g., ASHP Handbook on Injectable Drugs, Toissel, 18th ed. (2014).

In some embodiments, a pharmaceutical composition that includes a nucleic acid molecule encoding a polypeptide of the invention or a vector containing such nucleic acid molecule may be administered by way of gene delivery. Methods of gene delivery are well-known to one of skill in the art. Vectors that may be used for in vivo gene delivery and expression include, but are not limited to, retroviral vectors, adenoviral vectors, poxviral vectors (e.g., vaccinia viral vectors, such as Modified Vaccinia Ankara (MVA)), adeno-associated viral vectors, and alphaviral vectors. In some embodiments, mRNA molecules encoding polypeptides of the invention may be administered directly to a subject.

In some embodiments of the present invention, nucleic acid molecules encoding a polypeptide described herein or vectors containing such nucleic acid molecules may be administered using a hydrodynamic injection platform. In the hydrodynamic injection method, a nucleic acid molecule encoding a polypeptide described herein is put under the control of a strong promoter in an engineered plasmid (e.g., a viral plasmid). The plasmid is often delivered rapidly in a large fluid volume intravenously. Hydrodynamic injection uses controlled hydrodynamic pressure in veins to enhance cell permeability such that the elevated pressure from the rapid injection of the large fluid volume results in fluid and plasmid extravasation from the vein. The expression of the nucleic acid molecule is driven primarily by the liver. In mice, hydrodynamic injection is often performed by injection of the plasmid into the tail vein. In certain embodiments, mRNA molecules encoding a polypeptide described herein may be administered using hydrodynamic injection.

The dosage of the pharmaceutical compositions of the invention depends on factors including the route of administration, the disease to be treated, and physical characteristics, e.g., age, weight, general health, of the subject. A pharmaceutical composition of the invention may include a dosage of an ActRII signaling inhibitor of the invention ranging from 0.01 to 500 mg/kg (e.g., 0.01, 0.1, 0.2, 0.3, 0.325, 0.35, 0.375, 0.4, 0.5, 0.75, 1, 1.25, 1.5, 1.75, 2, 2.25, 2.5, 2.75, 3, 3.25, 3.5, 3.75, 4, 4.25, 4.5, 4.75, 5, 10, 15, 20, 25, 30, 35, 40, 45, 50, 100, 150, 200, 250, 300, 350, 400, 450, or 500 mg/kg) and, in a more specific embodiment, about 0.1 to about 30 mg/kg and, in a more specific embodiment, about 0.3 to about 30 mg/kg. The dosage may be adapted by the physician in accordance with conventional factors such as the extent of the disease and different parameters of the subject.

The pharmaceutical compositions are administered in a manner compatible with the dosage formulation and in such amount as is therapeutically effective to result in an improvement or remediation of the symptoms. The pharmaceutical compositions are administered in a variety of dosage forms, e.g., intravenous dosage forms, subcutaneous dosage forms, and oral dosage forms (e.g., ingestible solutions, drug release capsules). Generally, therapeutic proteins are dosed at 0.1-100 mg/kg, e.g., 0.5-50 mg/kg. Pharmaceutical compositions that include a polypeptide of the invention may be administered to a subject in need thereof, for example, one or more times (e.g., 1-10 times or more) daily, weekly, biweekly, every four weeks, monthly, bimonthly, quarterly, biannually, annually, or as medically necessary. In some embodiments, pharmaceutical compositions that include a polypeptide of the invention may be administered to a subject in need thereof weekly, biweekly, every four weeks, monthly, bimonthly, or quarterly. Dosages may be provided in either a single or multiple dosage regimens. The timing between administrations may decrease as the medical condition improves or increase as the health of the patient declines.

### Methods of Treatment

The ActRII signaling inhibitors described herein, such as the ActRIIA ligand traps containing the extracellular ActRIIA variants described herein which retain the beneficial properties of ActRIIA, such as low binding affinity to BMP9 and longer serum half-life as an Fc fusion protein, and gain some of the beneficial properties of ActRIIB, such as increased binding to activins A and B, can be used to disrupt endogenous activin signaling. Therefore, the ActRII signaling inhibitors, such as the ActRIIA ligand traps containing the extracellular ActRIIA variants, can be used to treat diseases or conditions in which activin signaling has been implicated. For example, activin receptor ligand, GDF11, has been found to be overexpressed in a mouse model of hemolytic anemia and associated with defects in red blood cell production. Signaling pathways involving activins also regulate hematopoiesis by preventing the differentiation of red blood cell, platelet, and neutrophil progenitor cells in order to maintain progenitor cells in a quiescent state. Without wishing to be bound by theory, a therapeutic agent that binds to activin receptor ligands (e.g., myostatin, activins, and/or GDF11) and reduces their binding to or interaction with endogenous activin receptors (e.g., by sequestering the endogenous ligands) or that binds to the endogenous receptors and disrupts their interactions with these ligands may have therapeutic utility for treating diseases or conditions involving ineffective hematopoiesis, such as cytopenias (e.g., anemia, thrombocytopenia, and/or neutropenia) associated with a myelodysplastic syndrome.

The ActRII signaling inhibitors described herein (e.g., an activin A antibody, a myostatin antibody, an activin B antibody, a GDF-11 antibody, an ActRII antibody, or an ActRII ligand trap, such as an ActRIIA ligand trap including an extracellular ActRIIA variant (e.g., an extracellular ActRIIA variant having the sequence of any one of SEQ ID NOs: 1-72 (e.g., SEQ ID NOs: 6-72), e.g., an effective amount of an ActRIIA ligand trap including an extracellular ActRIIA variant) can be used to treat a subject having or at risk of developing a cytopenia (e.g., anemia, thrombocytopenia, or neutropenia) associated with a myelodysplastic syndrome. The subject may be diagnosed as having a myelodysplastic syndrome according to the World Health Organization (WHO) classification or the French American British (FAB) classification. The myelodysplastic syndrome may be myelodysplastic syndrome with unilineage dysplasia (MDS-SLD), myelodysplastic syndrome with multilineage dysplasia (MDS-MLD), myelodysplastic syndrome with ring sideroblasts (MDS-RS, which includes single lineage dysplasia (MDS-RS-SLD) and multilineage dysplasia (MDS-RS-MLD)), myelodysplastic syndrome associated with isolated del chromosome abnormality (MDS with isolated del(5q)), myelodysplastic syndrome with excess blasts (MDS-EB; which includes myelodysplastic syndrome with excess blasts—type 1 (MDS-EB-1) and myelodysplastic syndrome with excess blasts—type 2 (MDS-EB-2)), myelodysplastic syndrome, unclassifiable (MDS-U), or myelodysplastic syndrome/myeloproliferative neoplasm with ring sideroblasts and thrombocytosis (MDS/MPN-RS-T). The myelodysplastic syndrome may be a very low, low, or intermediate risk myelodysplastic syndrome as determined by the Revised International Prognostic Scoring System (IPSS-R). The myelodysplastic syndrome may be an RS-positive myelodysplastic syndrome (e.g., the subject with a myelodysplastic syndrome may have ring sideroblasts) or a non-RS myelodysplastic syndrome (e.g., the subject with a myelodysplastic syndrome may lack ring sideroblasts). In some embodiments, the RS-positive myelodysplastic syndrome is associated with a splicing factor mutation, such as a mutation in SF3B1. In some embodiments, the MDS is associated with a defect in terminal maturation (often observed in RS-positive MDS and in subjects having splicing factor mutations, such a subject may have increased erythroid progenitor cells in the bone marrow relative to a healthy subject). In some embodiments, the MDS is associated with a defect in early-stage hematopoiesis (e.g., early-stage erythroid cell development, such as commitment or early differentiation, such a subject may have fewer erythroid progenitor cells in the bone marrow compared to a healthy subject or to a subject with a defect in terminal maturation). In some embodiments, the MDS is associated with elevated endogenous erythropoietin levels. In some embodiments, the myelodysplastic syndrome is associated with hypocellular bone marrow (e.g., a subject with MDS has hypocellular bone marrow). The subject may have a low transfusion burden or a high transfusion burden. In some embodiments, the subject has a low transfusion burden and received 1-3 RBC units in the eight weeks prior to treatment with an ActRII signaling inhibitor, such as an ActRII ligand trap including an ActRIIA variant described herein. In some embodiments, the subject has a low transfusion burden and did not receive a transfusion (received 0 RBC units) in the eight weeks prior to treatment with an ActRII signaling inhibitor, such as an ActRII ligand trap including ActRIIA variant described herein. In some embodiments, the subject does not respond well to erythropoietin (EPO) or is susceptible to adverse effects of EPO (e.g., hypertension, headaches, vascular thrombosis, influenza-like syndrome, obstruction of shunts, and myocardial infarction). The compositions and methods described herein can also be used to treat subjects that do not respond to an erythroid maturation agent. In some embodiments, the subject has previously been treated with an ESA. In some embodiments, the subject has not previously been treated with an ESA. In some embodiments, the subject has not previously been treated with azacitidine, decitabine, lenalidomide, luspatercept, or sotatercept. In some embodiments, the subject has an erythropoietin level greater than 100 mlU/mL. In some embodiments, the subject has an Eastern Cooperative Oncology Group (ECOG) performance score of less than or equal to two. In some embodiments, the subject has <5% blasts in bone marrow prior to treatment with an ActRII signaling inhibitor described herein. In some embodiments, the subject has a peripheral blood white blood cell count less than 13,000/μL prior to treatment with an ActRII signaling inhibitor described herein. In some embodiments, the subject has anemia. In some embodiments, the subject has thrombocytopenia. In some embodiments, the subject has both anemia and thrombocytopenia. In some embodiments, the subject has neutropenia. In some embodiments, the subject has anemia and neutropenia. In some embodiments, the subject has thrombocytopenia and neutropenia. In some embodiments, the subject has anemia, thrombocytopenia, and neutropenia.

The ActRII signaling inhibitors described herein (e.g., an activin A antibody, a myostatin antibody, an activin B antibody, a GDF-11 antibody, an ActRII antibody, or an ActRII ligand trap, such as an ActRIIA ligand trap including an extracellular ActRIIA variant (e.g., an extracellular ActRIIA variant having the sequence of any one of SEQ ID NOs: 1-72 (e.g., SEQ ID NOs: 6-72)), e.g., an effective amount of an ActRIIA ligand trap including an extracellular ActRIIA variant) can also be used to treat a subject having or at risk of developing a cytopenia (e.g., anemia, thrombocytopenia, or neutropenia) associated with chronic myelomonocytic leukemia (CMML). The CMML may be, for example, CMML-0, which is defined as CMML with less than 2% blasts in peripheral blood and less than 5% blasts in bone marrow with no Auer rods. In some embodiments, the cytopenia is anemia. In some embodiments, the cytopenia is thrombocytopenia. In some embodiments, the cytopenia is both anemia and thrombocytopenia. In some embodiments, the cytopenia is neutropenia. In some embodiments, the cytopenia is anemia and neutropenia. In some embodiments, the cytopenia is thrombocytopenia and neutropenia. In some embodiments, the cytopenia is anemia, thrombocytopenia, and neutropenia. In some embodiments, the subject has an enlarged spleen. The subject may have a high transfusion burden or a low transfusion burden and may be ring sideroblast positive or ring sideroblast negative.

The ActRII signaling inhibitors described herein (e.g., an activin A antibody, a myostatin antibody, an activin B antibody, a GDF-11 antibody, an ActRII antibody, or an ActRII ligand trap, such as an ActRIIA ligand trap including an extracellular ActRIIA variant (e.g., an extracellular ActRIIA variant having the sequence of any one of SEQ ID NOs: 1-72 (e.g., SEQ ID NOs: 6-72)), e.g., an effective amount of an ActRIIA ligand trap including an extracellular ActRIIA variant) can also be used to treat a subject having or at risk of developing a cytopenia (e.g., anemia, thrombocytopenia, or neutropenia) associated with myelofibrosis or to treat myelofibrosis (e.g., to treat multiple facets of the pathology of myelofibrosis). In some embodiments, the myelofibrosis is PMF, post-ET MF, or post-PV MF (e.g., diagnosed according to the 2017 World Health Organization criteria). In some embodiments, the subject has an Eastern Cooperative Oncology Group (ECOG) performance score of less than or equal to two. In some embodiments, the subject has anemia. Anemia is defined as hemoglobin ≤10 g/dL during screening, or receiving RBC transfusions. In some embodiments, the subject has thrombocytopenia. In some embodiments, the subject has both anemia and thrombocytopenia. In some embodiments, the subject has neutropenia. In some embodiments, the subject has anemia and neutropenia. In some embodiments, the subject has thrombocytopenia and neutropenia. In some embodiments, the subject has anemia, thrombocytopenia, and neutropenia. In some embodiments, the subject is ineligible for treatment with a JAK inhibitor (e.g., ruxolitinib, fedratinib, or pacritinib, for example, due to already having a cytopenia or due to myelofibrosis risk status). In some embodiments, the subject has discontinued treatment with a JAK inhibitor to due relapsed disease following treatment with a JAK inhibitor, being refractory to treatment with a JAK inhibitor, or intolerance to treatment with a JAK inhibitor, or no longer meeting the risk/benefit ratio to continue treatment with the JAK inhibitor. In some embodiments, the subject has a defect in JAK/STAT signaling (e.g., a reduction, deficiency, or failure in JAK/STAT signaling). In some embodiments, the myelofibrosis is intermediate or high-risk myelofibrosis. In some embodiments, the subject is identified as having a cytopenia (e.g., anemia, thrombocytopenia, or neutropenia) prior to administration of an ActRII signaling inhibitor described herein. In some embodiments, the method includes a step of identifying the subject as having a cytopenia (e.g., anemia, thrombocytopenia, or neutropenia) (e.g., by evaluating red blood cell, hemoglobin, hematocrit, platelet, and/or neutrophil levels) prior to administration of an ActRII signaling inhibitor described herein. The method can further include evaluating red blood cell, hemoglobin, hematocrit, reticulocyte, platelet, and/or neutrophil levels after administration of an ActRII signaling inhibitor described herein (e.g., 12 hours, 24 hours, 1, 2, 3, 4, 5, 6, or 7 days, 1, 2, 3, 4, 5, 6, 7, 8, 9, 10, 11, or 12 weeks, or 1, 2, 3, 4, 5, 6, 8, 10, 12, 18, or 24 months or more after the start of treatment with an ActRII signaling inhibitor described herein, such as by taking a CBC). In some embodiments, the subject does not receive concurrent treatment with an erythropoiesis stimulating agent (ESA), granulocyte colony-stimulating factor (G-CSF), granulocyte-macrophage colony-stimulating factor (GM-CSF), a thrombopoietin agonist (TPO), an immunomodulator imide drug (IMiD; e.g., thalidomide, pomalidomide, lenalidomide), interferon, or hydroxyurea, danazol, a steroid (other than prednisone of less than or equal to 10 mg/day or corticosteroid equivalent), a cytotoxic or chemotherapeutic agent, a hypomethylating agent, an RBC hematopoietic growth factor (e.g., Interleukin-3), an androgen, an oral retinoid, or arsenic trioxide.

The ActRII signaling inhibitors described herein (e.g., an activin A antibody, a myostatin antibody, an activin B antibody, a GDF-11 antibody, an ActRII antibody, or an ActRII ligand trap, such as an ActRIIA ligand trap including an extracellular ActRIIA variant (e.g., an extracellular ActRIIA variant having the sequence of any one of SEQ ID NOs: 1-72 (e.g., SEQ ID NOs: 6-72)), e.g., an effective amount of an ActRIIA ligand trap including an extracellular ActRIIA variant) can also be used to treat splenomegaly associated with extramedullary hematopoiesis.

The ActRII signaling inhibitors described herein (e.g., an activin A antibody, a myostatin antibody, an activin B antibody, a GDF-11 antibody, an ActRII antibody, or an ActRII ligand trap, such as an ActRIIA ligand trap including an extracellular ActRIIA variant (e.g., an extracellular ActRIIA variant having the sequence of any one of SEQ ID NOs: 1-72 (e.g., SEQ ID NOs: 6-72)), e.g., an effective amount of an ActRIIA ligand trap including an extracellular ActRIIA variant) can also be used to reduce platelet number or platelet volume in a subject with high platelet levels, such as a subject with myelofibrosis, thrombocythemia, or polycythemia vera, or a subject who requires phlebotomy due to excess red blood cells. Accordingly, the methods described herein can also be used to treat a subject with thrombocythemia or polycythemia vera, or a subject who requires phlebotomy due to excess red blood cells.

The ActRII signaling inhibitors described herein (e.g., an activin A antibody, a myostatin antibody, an activin B antibody, a GDF-11 antibody, an ActRII antibody, or an ActRII ligand trap, such as an ActRIIA ligand trap including an extracellular ActRIIA variant (e.g., an extracellular ActRIIA variant having the sequence of any one of SEQ ID NOs: 1-72 (e.g., SEQ ID NOs: 6-72)), e.g., an effective amount of an ActRIIA ligand trap including an extracellular ActRIIA variant) can also be used to treat cytopenias having other causes. For example, the ActRII signaling inhibitors can be used to treat a cytopenia (e.g., anemia, thrombocytopenia, and/or neutropenia) that results from treatment with an antifungal agent (e.g., ketoconazole, terbinafine, fluconazole, micafungin, or caspofungin) or an immunosuppressant (e.g., azathioprine, methotrexate, or mycophenolate mofetil). The ActRII signaling inhibitors can also be used to treat anemia that results from treatment with an antibiotic (e.g., a cephalosporin or a penicillin). In addition, the ActRII signaling inhibitors described herein (e.g., an activin A antibody, a myostatin antibody, an activin B antibody, a GDF-11 antibody, an ActRII antibody, or an ActRII ligand trap, such as an ActRIIA ligand trap including an extracellular ActRIIA variant (e.g., an extracellular ActRIIA variant having the sequence of any one of SEQ ID NOs: 1-72 (e.g., SEQ ID NOs: 6-72)), e.g., an effective amount of an ActRIIA ligand trap including an extracellular ActRIIA variant) can be used to improve hematopoietic stem cell engraftment (e.g., when administered to a subject prior to or concurrent with hematopoietic stem cell transplant).

In some embodiments, the methods described herein affect myostatin, activin A, activin B, and/or BMP9 signaling (e.g., reduce or inhibit the binding of activin A, activin B, myostatin, and/or BMP9 to their endogenous receptors, e.g., ActRIIA, ActRIIB, and/or BMPRII) in the subject. In some embodiments, the methods described herein increase hemoglobin levels, increase hematocrit, increase red blood cell count, increase red blood cell volume, increase red cell mass, increase reticulocytes, increase proerythroblasts, increase or induce red blood cell formation or production, increase the maturation and/or differentiation of erythroid progenitors (early or late- (e.g., terminal) stage progenitors, e.g., early-stage erythroid progenitors, such burst forming unit-erythroid cells (BFU-Es) and/or colony forming unit-erythroid cells (CFU-Es), e.g., increase the maturation and/or differentiation of BFU-Es and/or CFU-Es into proerythroblasts, reticulocytes, or red blood cells, e.g., increase proerythroblast and/or reticulocyte numbers), increase late-stage erythroid precursor maturation (e.g., terminal maturation, such as the maturation of reticulocytes into red blood cells, or the maturation of erythroblasts into reticulocytes and/or red blood cells), recruit early-stage progenitors into the erythroid lineage, increase the number of early-stage erythroid precursors and/or progenitors (e.g., expand the early-stage precursor population to provide a continuous supply of precursors to replenish polychromatic erythroblasts and allow for a continuous supply of maturing reticulocytes), promote the progression of erythroid precursors and/or progenitors through erythropoiesis, reduce the accumulation of red blood cell progenitor cells (e.g., by stimulating progenitor cells to progress to maturation), increase platelet levels (e.g., increase platelet count), increase or induce megakaryocyte differentiation and/or maturation (e.g., to produce platelets, e.g., terminal maturation of pro-platelets to platelets), reduce platelet progenitor accumulation (e.g., by stimulating progenitor cells to progress to maturation), increase megakaryocyte progenitors (e.g., increase megakaryocyte progenitor renewal), increase pro-platelets, promote or increase platelet formation or production, increase neutrophil levels (e.g., increase neutrophil count), increase or induce the differentiation and/or maturation of progenitor cells (e.g., myeloid progenitors, myeloblasts, or myelocytes) into neutrophils, and/or induce or increase neutrophil formation or production in the subject. In some embodiments, the methods described herein increase the rate of recovery from thrombocytopenia. These changes may be observed in a subject treated with an ActRII signaling inhibitor described herein (e.g., an activin A antibody, a myostatin antibody, an activin B antibody, a GDF-11 antibody, an ActRII antibody, or an ActRII ligand trap, such as an ActRIIA ligand trap including an extracellular ActRIIA variant (e.g., an extracellular ActRIIA variant having the sequence of any one of SEQ ID NOs: 1-72 (e.g., SEQ ID NOs: 6-72)), e.g., an effective amount of an ActRIIA ligand trap including an extracellular ActRIIA variant) compared to measurements obtained prior to treatment or compared to measurements obtained from untreated subjects having the same disease or condition (e.g., an MDS-associated cytopenia, a CMML-associated cytopenia, or a myelofibrosis-associated cytopenia). In some embodiments, the methods described herein reduce spleen size (e.g., in a subject having an enlarged spleen, such as an enlarged spleen associated with CMML or myelofibrosis). In some embodiments, the methods described herein improve or restore hematopoiesis in the bone marrow, reduce or reverse reticulin and/or collagen deposition, or reverse bony changes associated with myelofibrosis. In some embodiments, the methods described herein reduce or ameliorate megakaryocyte dysfunction (e.g., megakaryocyte dysfunction in the bone marrow in a subject with myelofibrosis), which may prevent or reduce inflammation/fibrosis, restore hematopoiesis in the bone marrow, and treat cytopenias due to myelofibrosis and those that result from JAK inhibitor treatment. In some embodiments, the methods described herein reduce or resolve hepatosplenomegaly or splenomegaly (e.g., reduce spleen volume and/or splenic extra medullary hematopoiesis) and its symptoms in a subject with myelofibrosis. In some embodiments, the methods described herein reduce bone marrow fibrosis and alleviate the symptoms caused by the loss of bone marrow function in a subject with myelofibrosis. In some embodiments, the methods described herein slow or reduce the progression of bone marrow fibrosis. In some embodiments, the methods described herein improve or ameliorate the attenuated bone resorption and osteosclerosis in patients with myelofibrosis. In some embodiments, the methods described herein improve fibrosis, bone histomorphology, spleen size (e.g., reduce spleen size), myelofibrosis symptoms, bone marrow fibrosis, and/or osteosclerotic dysplasia in a subject with myelofibrosis. In some embodiments, the methods described herein reduce bleeding events. In some embodiments, the methods described herein decrease infections.

In some embodiments, the compositions and methods described herein reduce the need of a subject with MDS or CMML for a blood transfusion (e.g., reduce transfusion burden, for example, the subject no longer needs blood transfusions, or the subject needs less frequent blood transfusion than before treatment with the compositions and methods described herein). In some embodiments, the compositions and methods described herein promote transfusion independence in a subject with MDS or CMML (e.g., a subject who required 1 or more (e.g., 2, 3, 4, 5, 6, 7, 8, 9, 10, or more) RBC units over 8 weeks directly preceding treatment initiation does not require a transfusion for at least 8 weeks, 10 weeks, 12 weeks, 14 weeks, 16 weeks, 18 weeks, 20 weeks, 22 weeks, 24 weeks, 26, weeks, 1 year, 2 years or more during treatment with an ActRII signaling inhibitor, such as an ActRIIA ligand trap including an ActRIIA variant described herein). For MDS and CMML, RBC transfusions are recommended when hemoglobin is <9.0 g/dL, and may be recommended if Hgb is ≥9.0 g/dL and associated with symptom(s) of anemia (e.g., hemodynamic or pulmonary compromise requiring treatment) or comorbidity justifying a threshold ≥9.0 g/dL Hgb. A complete blood count (CBC) can be taken to assess the response of a subject to treatment with a composition described herein, and hemoglobin levels can be reviewed to determine whether the subject has a stable hemoglobin level above the transfusion threshold. In subjects who achieve transfusion independence, both hemoglobin levels and absolute reticulocyte counts may increase. In some embodiments, the compositions and methods described herein slow or inhibit the progression of lower-risk MDS to higher-risk MDS and/or the progression of lower-risk MDS or CMML to acute myeloid leukemia (AML). For example, treatment of anemia in a subject having very low, low, or intermediate risk MDS and a low transfusion burden may lead to a hemoglobin increase of greater than or equal to 1.5 g/dL from baseline or pretreatment measurements (e.g., for at least one day, two days, three days, four days, five days, six days, one week, two weeks, three weeks, four weeks, six weeks, eight weeks, ten weeks, twelve weeks, fourteen weeks, sixteen weeks, eighteen weeks, twenty weeks, twenty two weeks, twenty four weeks, twenty six weeks, one year, two years, or longer during treatment with an ActRII signaling inhibitor, such as an ActRIIA ligand trap including an extracellular ActRIIA variant described herein). In another example, treatment of anemia in a subject having very low, low, or intermediate risk MDS and a high transfusion burden may lead to a reduction of ≥50% or ≥4 RBC units transfused compared to pretreatment (e.g., comparing an eight-week period during treatment to an eight-week period prior to treatment). In yet another example, treatment of anemia in a subject having very low, low, or intermediate risk MDS and a low transfusion burden (e.g., a subject who received 1-3 RBC units in the eight weeks prior to treatment with an ActRII signaling inhibitor, such as an ActRIIA ligand trap including an extracellular ActRIIA variant described herein) or a high transfusion burden (e.g., a subject who received 4 or more RBC units in the eight weeks prior to treatment with an ActRII signaling inhibitor, such as an ActRIIA ligand trap including ActRIIA variant described herein) may lead to transfusion independence (e.g., transfusion independence for at least eight weeks, ten weeks, twelve weeks, fourteen weeks, sixteen weeks, eighteen weeks, twenty weeks, twenty two weeks, twenty four weeks, twenty six weeks, one year, two years, or longer during treatment with an ActRII signaling inhibitor, such as an ActRIIA ligand trap including an extracellular ActRIIA variant described herein, e.g., comparing an eight-week period during treatment to the eight-week period immediately prior to treatment). In some embodiments, treatment according to the methods described herein leads to a mean platelet increase from baseline of greater than 30×10⁹/L in the absence of platelet transfusions (for a subject with a baseline value greater than 20×10⁹/L). In some embodiments, treatment according to the methods described herein leads to an increase of greater than or equal to 100% and greater than 500/μL from pretreatment neutrophil count. In some embodiments, treatment according to the methods described herein results in a change in baseline in the Functional Assessment of Cancer Therapy-Anemia (FACT-An), Quality of Life in Myelodysplasia Scale (QUALMS), or Functional Assessment of Chronic Illness Therapy-Fatigue (FACIT-F) questionnaire. In some embodiments, the subject is identified as having an MDS-associated cytopenia (e.g., anemia, thrombocytopenia, or neutropenia) prior to treatment with an ActRIIA variant described herein. In some embodiments, the method includes a step of identifying the subject as having an MDS-associated cytopenia (e.g., anemia, thrombocytopenia, or neutropenia) (e.g., by evaluating red blood cell, hemoglobin, hematocrit, platelet, and/or neutrophil levels) prior to treatment with an ActRII signaling inhibitor, such as an ActRIIA ligand trap including an ActRIIA variant described herein. The method can further include evaluating red blood cell, hemoglobin, hematocrit, reticulocyte, platelet, and/or neutrophil levels after administration of an ActRII signaling inhibitor, such as an ActRIIA ligand trap including an ActRIIA variant described herein (e.g., 12 hours, 24 hours, 1, 2, 3, 4, 5, 6, or 7 days, 1, 2, 3, 4, 5, 6, 7, 8, 9, 10, 11, or 12 weeks, or 1, 2, 3, 4, 5, 6, 8, 10, 12, 18, or 24 months or more after treatment initiation, such as by taking a CBC).

In some embodiments, treatment according to methods described herein leads to a mean hemoglobin increase of greater than or equal to 1.5 g/dL or 2.0 g/dL from baseline or pretreatment measurements over a period of 12 consecutive weeks or more, such as 12 weeks, 14 weeks, 16 weeks, 18 weeks, 20 weeks, 22 weeks, 24, weeks, 26 weeks, 1 year, 2 years or more, during treatment with an ActRII signaling inhibitor described herein, for example during the first 24 weeks or 52 weeks of treatment of a transfusion-independent subject according to the methods described herein for myelofibrosis. In some embodiments, treatment according to methods described herein leads to a decrease of one or more in the brief fatigue inventory score from baseline within the first 24 weeks or 52 weeks of treatment of a transfusion-independent subject according to the methods described herein for myelofibrosis. In some embodiments, the methods described herein reduce the need of a subject, such as a subject with anemia and myelofibrosis requiring RBC transfusions, for a blood transfusion (e.g., reduce transfusion burden, for example, the subject no longer needs blood transfusions, or the subject needs less frequent blood transfusion than before treatment with the compositions and methods described herein). In some embodiments, treatment according to the methods described herein reduces the number of RBC transfusions from baseline pre-treatment measurements (e.g., measurements taken over 12 weeks directly preceding treatment initiation with an ActRII signaling inhibitor described herein) for a period of 12 consecutive weeks of more, such as 12 weeks, 14 weeks, 16 weeks, 18 weeks, 20 weeks, 22 weeks, 24, weeks, 26 weeks, 1 year, 2 years or more, during treatment with an ActRII signaling inhibitor described herein, for example during the first 24 weeks or 52 weeks of treatment according to the methods described herein for myelofibrosis). In some embodiments, the compositions and methods described herein promote transfusion independence (e.g., a subject who required 1 or more (e.g., 2, 3, 4, 5, 6, 7, 8, 9, 10, or more) RBC units over 12 weeks directly preceding treatment initiation with an ActRII signaling inhibitor described herein does not require a transfusion for 12 consecutive weeks of more, such as 12 weeks, 14 weeks, 16 weeks, 18 weeks, 20 weeks, 22 weeks, 24, weeks, 26 weeks, 1 year, 2 years or more, during treatment with an ActRII signaling inhibitor described herein, for example during the first 24 weeks or 52 weeks of treatment according to the methods described herein for myelofibrosis). Concurrent treatment for anemia with RBC transfusions is recommended for a subject with myelofibrosis when hemoglobin is <8.0 g/dL, and may be recommended if Hgb is ≥8.0 g/dl and associated with symptom(s) of anemia (e.g., hemodynamic or pulmonary compromise requiring treatment) or comorbidity justifying a threshold ≥8.0 g/dL Hgb. A complete blood count (CBC) can be taken to assess the response of a subject to treatment with a composition described herein, and hemoglobin levels can be reviewed to determine whether the subject has a stable hemoglobin level above the transfusion threshold. In subjects who achieve transfusion independence, both hemoglobin levels and absolute reticulocyte counts may increase. In some embodiments, treatment according to the methods described herein leads to an improvement in the Myelofibrosis Symptom Assessment Form Total Symptom Score (MF-SAF-TSS) of greater than or equal to 50% from baseline (e.g., at least 50%, 55%, 60%, 65%, 70%, 75%, 80%, 85%, 90% or more from baseline), such as by 24 weeks or 52 weeks of treatment according to the methods described herein. In some embodiments, treatment according to the methods described herein leads to a decrease in spleen volume of greater than or equal to 35% from baseline (e.g., at least 35%, 40%, 45%, 50%, 55%, 60%, 65%, 70%, 75% or more from baseline) as measured by computed tomography, such as by 24 weeks or 52 weeks of treatment according to the methods described herein for myelofibrosis. In some embodiments, the compositions and methods described herein slow or inhibit the progression to acute myeloid leukemia (AML) (bone marrow blasts >20%) and/or accelerated myelofibrosis (bone marrow blasts ≥10%), such as by 24 weeks or 52 weeks of treatment according to the methods described herein. In some embodiments, treatment according to the methods described herein leads to a mean platelet increase from baseline of greater than 30×10⁹/L for 12 weeks or more, such as 12 weeks, 14 weeks, 16 weeks, 18 weeks, 20 weeks, 22 weeks, 24, weeks, 26 weeks, 1 year, 2 years or more, during treatment with an ActRII signaling inhibitor described herein (in the absence of platelet transfusions), for example by 24 weeks or 52 weeks of treatment according to the methods described herein for myelofibrosis. In some embodiments, treatment according to the methods described herein reduces episodes of anemia, neutropenia, and thrombocytopenia of ≥Grade 1. In some embodiments, treatment according to the methods described herein reduces osteosclerosis from baseline as assessed using CT, such as by 24 weeks or 52 weeks of treatment as described herein for myelofibrosis. In some embodiments, treatment according to the methods described herein leads to a decrease in Patient Reported Outcomes Measurement Information System (PROMIS) score or BFI score from baseline, such as by 24 weeks or 52 weeks of treatment as described herein for myelofibrosis. In some embodiments, treatment according to the methods described herein slows or reduces the progression of bone marrow fibrosis or improves (e.g., reverses) bone marrow fibrosis. For example, treatment according to the methods described herein may lead to an improvement in bone marrow fibrosis grade from baseline or may prevent bone marrow fibrosis grade from worsening, such as by 24 weeks or 52 weeks of treatment as described herein. Treatment according to the methods described herein may also increase red cell parameters, such as reticulocyte count, mean corpuscular volume (MCV), mean corpuscular hemoglobin (MCH), and reticulocyte cell hemoglobin, and/or biomarkers of blood cell production, such as erythropoietin (EPO) and thrombopoietin (TPO) levels. In some embodiments, treatment according to the methods described herein increases mean platelet volume and immature platelet fraction. In some embodiments, treatment according to the methods described herein increases FSH compared to baseline. In some embodiments, treatment according to the methods described herein increases biomarkers of bone metabolism compared to baseline, such as bone specific alkaline phosphatase (BSAP) and serum C-telopeptide of type I collagen (CTX). In some embodiments, treatment according to the methods described herein reduces the development of myelofibrosis-associated molecular and cytogenic abnormalities over the duration of treatment.

Treatment according to the methods described herein may also lead to changes in biomarkers of iron metabolism (e.g., serum iron, ferritin, transferrin, transferrin saturation, total iron binding capacity, soluble transferrin receptor level, and hepcidin), dose of iron chelators, and cytokine levels compared to baseline, such as by 8 weeks, 12 weeks, 20 weeks, 24 weeks, 26 weeks, 52 weeks, or 2 years of treatment as described herein. For example, treatment according to the methods described herein (e.g., treatment with an ActRII signaling inhibitor described herein, such as an ActRIIA ligand trap including an extracellular ActRIIA variant (e.g., an extracellular ActRIIA variant having the sequence of any one of SEQ ID NOs: 1-72 (e.g., SEQ ID NOs: 6-72))) can lead to a reduction in serum ferritin and in increase in soluble transferrin receptor (sTfR). Ferritin is expected to decrease as patients become transfusion-independent and ineffective erythropoiesis improves, as iron stores are incorporated into new red blood cells. An increase in sTfR is a surrogate marker for induction of erythropoiesis, and measurement of sTfR may be used as a pharmacodynamic marker to monitor changes in erythropoiesis in response to therapy, potentially even before changes in hemoglobin are apparent.

In some embodiments, the methods described herein (e.g., the methods of treating an MDS-associated cytopenia, CMML-associated cytopenia, or myelofibrosis-associated cytopenia described herein) do not cause any vascular complications in the subject, such as increased vascular permeability or leakage.

In some embodiments the ActRII signaling inhibitor that is used in the methods described herein is an ActRIIA ligand trap including an extracellular ActRIIA variant (e.g., an extracellular ActRIIA variant having the sequence of any one of SEQ ID NOs: 1-72 (e.g., SEQ ID NOs: 6-72)). In some embodiments, the ActRIIA ligand trap including an extracellular ActRIIA variant is administered at a dosage ranging from 0.01 to 500 mg/kg (e.g., 0.01, 0.1, 0.2, 0.3, 0.325, 0.35, 0.375, 0.4, 0.5, 0.75, 1, 1.25, 1.5, 1.75, 2, 2.25, 2.5, 2.75, 3, 3.25, 3.5, 3.75, 4, 4.25, 4.5, 4.75, 5, 10, 15, 20, 25, 30, 35, 40, 45, 50, 100, 150, 200, 250, 300, 350, 400, 450, or 500 mg/kg) and, in a more specific embodiment, about 0.1 to about 30 mg/kg and, in a more specific embodiment, about 0.3 to about 30 mg/kg. In any of the methods described herein an ActRIIA ligand trap including an extracellular ActRIIA variant (e.g., an extracellular ActRIIA variant having the sequence of any one of SEQ ID NOs: 1-71 (e.g., SEQ ID NOs: 6-71) that further includes a C-terminal extension of one or more amino acids (e.g., 1, 2, 3, 4, 5, 6 or more amino acids) may be used as the therapeutic protein. In any of the methods described herein, a dimer (e.g., homodimer or heterodimer) formed by the interaction of two Fc domain monomers that are each fused to a polypeptide including an extracellular ActRIIA variant (e.g., an extracellular ActRIIA variant having the sequence of any one of SEQ ID NOs: 1-72 (e.g., SEQ ID NOs: 6-72) may be used as the therapeutic protein. In any of the methods described herein, an ActRIIA ligand trap including an extracellular ActRIIA variant (e.g., an extracellular ActRIIA variant having the sequence of any one of SEQ ID NOs: 1-72 (e.g., SEQ ID NOs: 6-72)) fused to a moiety (e.g., an Fc domain monomer, an Fc domain, an albumin-binding peptide, a fibronectin domain, or a human serum albumin) may be used as the therapeutic protein. Nucleic acids encoding the polypeptides described herein, or vectors containing said nucleic acids can also be administered according to any of the methods described herein. In any of the methods described herein, the polypeptide, nucleic acid, or vector can be administered as part of a pharmaceutical composition.

Compositions that can be administered to a subject according to the methods described herein are provided in Tables 18-21, below.

TABLE 18

| Row | Composition |
|---|---|
| 1 | A polypeptide comprising an extracellular activin receptor |
| | type IIa (ActRIIa) variant, the variant |
| | having a sequence of |
| | GAILGRSETQECLX₁X₂NANWX₃X₄X₅X₆TNQTGVEX₇CX₈GX₉X₁₀X₁₁X₁₂X₁₃X₁₄HCX₁₅ATWX₁₆NI |
| | SGSIEIVX₁₇X₁₈GCX₁₉X₂₀X₂₁DX₂₂NCYDRTDCVEX₂₃X₂₄X₂₅X₂₆PX₂₇VYFCCCEGNMCNEKFSYF |
| | PEMEVTQPTS (SEQ ID NO: 1), |
| | wherein X₁ is F or Y; X₂ is F or Y; X₃ is E or A; X₄ is K or L; |
| | X₅ is D or E; X₆ is R or A; X₇ is P or |
| | R; X₈ is Y or E; X₉ is D or E; X₁₀ is K or Q; X₁₁ is D or A; |
| | X₁₂ is K or A; X₁₃ is R or A; X₁₄ is R or |
| | L; X₁₅ is F or Y; X₁₆ is K, R, or A; X₁₇ is K, A, Y, F, or I; |
| | X₁₈ is Q or K; X₁₉ is W or A; X₂₀ is L or |
| | A; X₂₁ is D, K, R, A, F, G, M, N, or I; X₂₂ is I, F, or A; |
| | X₂₃ is K or T; X₂₄ is K or E; X₂₅ is D or E; |
| | X₂₆ is S or N; and X₂₇ is E or Q, and |
| | wherein the variant has at least one amino acid substitution |
| | relative to a wild-type extracellular |
| | ActRIIa having the sequence of SEQ ID NO: 73. |
| 2 | The polypeptide of row 1, |
| | wherein the variant has a sequence of |
| | GAILGRSETQECLFX₂NANWX₃X₄X₅X₆TNQTGVEX₇CX₈GX₉KX₁₁X₁₂X₁₃X₁₄HCX₁₅ATWX₁₆NIS |
| | GSIEIVX₁₇ |
| | X₁₈GCX₁₉X₂₀X₂₁DX₂₂NCYDRTDCVEX₂₃X₂₄X₂₅X₂₆PX₂₇VYFCCCEGNMCNEKFSYFPEMEVTQP |
| | TS (SEQ ID NO: 2). |
| 3 | The polypeptide of row 1 or 2, |
| | wherein the variant has a sequence of |
| | GAILGRSETQECLFX₂NANWEX₄X₅RTNQTGVEX₇CX₈GX₉KDKRX₁₄HCX₁₅ATWX₁₆NISGSIEIV |
| | KX₁₈GCWLDDX₂₂NCYDRTDCVEX₂₃X₂₄X₂₅X₂₆PX₂₇VYFCCCEGNMCNEKFSYFPEMEVTQPTS |
| | (SEQ ID NO: 3). |
| 4 | The polypeptide of any one of rows 1-3, |
| | wherein the variant has a sequence of |
| | GAILGRSETQECLFX₂NANWEX₄DRTNQTGVEX₇CX₈GX₉KDKRX₁₄HCX₁₅ATWX₁₆NISGSIEIV |
| | KX₁₈GCWLDDX₂₂NCYDRTDCVEX₂₃KX₂₅X₂₆PX₂₇VYFCCCEGNMCNEKFSYFPEMEVTQPTS |
| | (SEQ ID NO: 4). |
| 5 | The polypeptide of any one of rows 1-4, |
| | wherein the variant has a sequence of |
| | GAILGRSETQECLFX₂NANWEX₄DRTNQTGVEPCX₈GX₉KDKRX₁₄HCFATWKNISGSIEIVKX₁₈ |
| | GCWLDDINCYDRTDCVEX₂₃KX₂₅X₂₆PX₂₇VYFCCCEGNMCNEKFSYFPEMEVTQPTS (SEQ |
| | ID NO: 5). |
| 6 | The polypeptide of row 1, |
| | wherein X₁ is F. |
| 7 | The polypeptide of row 1, |
| | wherein X₁ is Y. |
| 8 | The polypeptide of row 1, 6, or 7 |
| | wherein X₁₀ is K. |
| 9 | The polypeptide of row 1, 6, or 7 |
| | wherein X₁₀ is Q. |
| 10 | The polypeptide of any one of rows 1-9, |
| | wherein X₂ is F. |
| 11 | The polypeptide of any one of rows 1-9, |
| | wherein X₂ is or Y. |
| 12 | The polypeptide of any one of rows 1, 2, and 6-11, |
| | wherein X₃ is E. |
| 13 | The polypeptide of any one of rows 1, 2, and 6-11, |
| | wherein X₃ is A. |
| 14 | The polypeptide of any one of rows 1-13, |
| | wherein X₄ is K. |
| 15 | The polypeptide of any one of rows 1-13, |
| | wherein X₄ is L. |
| 16 | The polypeptide of any one of rows 1, 2, 3, and 6-15, |
| | wherein X₅ is D. |
| 17 | The polypeptide of any one of rows 1, 2, 3, and 6-15, |
| | wherein X₅ is E. |
| 18 | The polypeptide of any one of rows 1, 2 and 6-17, |
| | wherein X₆ is R. |
| 19 | The polypeptide of any one of rows 1, 2 and 6-17, |
| | wherein X₆ is A. |
| 20 | The polypeptide of any one of rows 1-4 and 6-19, |
| | wherein X₇ is P. |
| 21 | The polypeptide of any one of rows 1-4 and 6-19, |
| | wherein X₇ is R. |
| 22 | The polypeptide of any one of rows 1-21, |
| | wherein X₈ is Y. |
| 23 | The polypeptide of any one of rows 1-21, |
| | wherein X₈ is E. |
| 24 | The polypeptide of any one of rows 1-23, |
| | wherein X₉ is D. |
| 25 | The polypeptide of any one of rows 1-23, |
| | wherein X₉ is E. |
| 26 | The polypeptide of any one of rows 1, 2 and 6-25, |
| | wherein X₁₁ is D. |
| 27 | The polypeptide of any one of rows 1, 2 and 6-25, |
| | wherein X₁₁ is A. |
| 28 | The polypeptide of any one of rows 1, 2 and 6-27, |
| | wherein X₁₂ is K. |
| 29 | The polypeptide of any one of rows 1, 2 and 6-27, |
| | wherein X₁₂ is A. |
| 30 | The polypeptide of any one of rows 1, 2 and 6-29, |
| | wherein X₁₃ is R. |
| 31 | The polypeptide of any one of rows 1, 2 and 6-29, |
| | wherein X₁₃ is A. |
| 32 | The polypeptide of any one of rows 1-31, |
| | wherein X₁₄ is R. |
| 33 | The polypeptide of any one of rows 1-31, |
| | wherein X₁₄ is L. |
| 34 | The polypeptide of any one of rows 1-4 and 6-33, |
| | wherein X₁₅ is F. |
| 35 | The polypeptide of any one of rows 1-4 and 6-33, |
| | wherein X₁₅ is Y. |
| 36 | The polypeptide of any one of rows 1-4 and 6-35, |
| | wherein X₁₆ is K. |
| 37 | The polypeptide of any one of rows 1-4 and 6-35, |
| | wherein X₁₆ is R. |
| 38 | The polypeptide of any one of rows 1-4 and 6-35, |
| | wherein X₁₆ is A. |
| 39 | The polypeptide of any one of rows 1, 2 and 6-38, |
| | wherein X₁₇ is K. |
| 40 | The polypeptide of any one of rows 1, 2 and 6-38, |
| | wherein X₁₇ is A. |
| 41 | The polypeptide of any one of rows 1, 2 and 6-38, |
| | wherein X₁₇ is Y. |
| 42 | The polypeptide of any one of rows 1, 2 and 6-38, |
| | wherein X₁₇ is F. |
| 43 | The polypeptide of any one of rows 1, 2 and 6-38, |
| | wherein X₁₇ is I. |
| 44 | The polypeptide of any one of rows 1-43, |
| | wherein X₁₈ is Q. |
| 45 | The polypeptide of any one of rows 1-43, |
| | wherein X₁₈ is K. |
| 46 | The polypeptide of any one of rows 1, 2 and 6-45, |
| | wherein X₁₉ is W. |
| 47 | The polypeptide of any one of rows 1, 2 and 6-45, |
| | wherein X₁₉ is A. |
| 48 | The polypeptide of any one of rows 1, 2 and 6-47, |
| | wherein X₂₀ is L. |
| 49 | The polypeptide of any one of rows 1, 2 and 6-47, |
| | wherein X₂₀ is A. |
| 50 | The polypeptide of any one of rows 1, 2 and 6-49, |
| | wherein X₂₁ is D. |
| 51 | The polypeptide of any one of rows 1, 2 and 6-49, |
| | wherein X₂₁ is K. |
| 52 | The polypeptide of any one of rows 1, 2 and 6-49, |
| | wherein X₂₁ is R. |
| 53 | The polypeptide of any one of rows 1, 2 and 6-49, |
| | wherein X₂₁ is A. |
| 54 | The polypeptide of any one of rows 1, 2 and 6-49, |
| | wherein X₂₁ is F. |
| 55 | The polypeptide of any one of rows 1, 2 and 6-49, |
| | wherein X₂₁ is G. |
| 56 | The polypeptide of any one of rows 1, 2 and 6-49, |
| | wherein X₂₁ is M. |
| 57 | The polypeptide of any one of rows 1, 2 and 6-49, |
| | wherein X₂₁ is N. |
| 58 | The polypeptide of any one of rows 1, 2 and 6-49, |
| | wherein X₂₁ is I. |
| 59 | The polypeptide of any one of rows 1-4 and 6-58, |
| | wherein X₂₂ is I. |
| 60 | The polypeptide of any one of rows 1-4 and 6-58, |
| | wherein X₂₂ is F. |
| 61 | The polypeptide of any one of rows 1-4 and 6-58, |
| | wherein X₂₂ is A. |
| 62 | The polypeptide of any one of rows 1-61, |
| | wherein X₂₃ is K. |
| 63 | The polypeptide of any one of rows 1-61, |
| | wherein X₂₃ is T. |
| 64 | The polypeptide of any one of rows 1, 2, 3, and 6-63, |
| | wherein X₂₄ is K. |
| 65 | The polypeptide of any one of rows 1, 2, 3, and 6-63, |
| | wherein X₂₄ is E. |
| 66 | The polypeptide of any one of rows 1-65, |
| | wherein X₂₅ is D. |
| 67 | The polypeptide of any one of rows 1-65, |
| | wherein X₂₅ is E. |
| 68 | The polypeptide of any one of rows 1-67, |
| | wherein X₂₆ is S. |
| 69 | The polypeptide of any one of rows 1-67, |
| | wherein X₂₆ is N. |
| 70 | The polypeptide of any one of rows 1-69, |
| | wherein X₂₇ is E. |
| 71 | The polypeptide of any one of rows 1-69, |
| | wherein X₂₇ is Q. |
| 72 | The polypeptide of any one of rows 1-71, |
| | wherein X₂₃ is T, X₂₄ is E, X₂₅ is E, and X₂₆ is N. |
| 73 | The polypeptide of any one of rows 1-71, |
| | wherein X₂₃ is T, X₂₄ is K, X₂₅ is E, and X₂₆ is N. |
| 74 | The polypeptide of any one of rows 1-73, |
| | wherein X₁₇ is K. |
| 75 | The polypeptide of row 1, |
| | wherein the variant has the sequence of any one of SEQ ID NOs: 6- |
| | 72. |
| 76 | The polypeptide of row 75, |
| | wherein the variant has the sequence of SEQ ID NO: 69. |
| 77 | The polypeptide of row 75, |
| | wherein the variant has the sequence of SEQ ID NO: 58. |
| 78 | The polypeptide of row 75, |
| | wherein the variant has the sequence of SEQ ID NO: 6. |
| 79 | The polypeptide of row 75, |
| | wherein the variant has the sequence of SEQ ID NO: 38. |
| 80 | The polypeptide of row 75, |
| | wherein the variant has the sequence of SEQ ID NO: 41. |
| 81 | The polypeptide of row 75, |
| | wherein the variant has the sequence of SEQ ID NO: 44. |
| 82 | The polypeptide of row 75, |
| | wherein the variant has the sequence of SEQ ID NO: 70. |
| 83 | The polypeptide of row 75, |
| | wherein the variant has the sequence of SEQ ID NO: 71. |
| 84 | The polypeptide of row 75, |
| | wherein the variant has the sequence of SEQ ID NO: 72. |
| 85 | The polypeptide of any one of rows 1-84, |
| | wherein the amino acid at position X₂₄ is replaced |
| | with the amino acid K. |
| 86 | The polypeptide of any one of rows 1-85, |
| | wherein the amino acid at position X₂₄ is replaced |
| | with the amino acid E. |
| 87 | The polypeptide of any one of rows 1-86, further |
| | comprising a C-terminal extension of one or |
| | more amino acids. |
| 88 | The polypeptide of row 87, |
| | wherein the C-terminal extension is NP. |
| 89 | The polypeptide of row 87, |
| | wherein the C-terminal extension is NPVTPK (SEQ ID NO: 78). |
| 90 | The polypeptide of any one of rows 1-89, further |
| | comprising an Fc domain monomer fused to |
| | the C-terminus of the polypeptide by way of a linker. |
| 91 | The polypeptide of row 90, |
| | wherein the Fc domain monomer comprises the sequence of SEQ |
| | ID NO: 97. |
| 92 | The polypeptide of any one of rows 1-89, further |
| | comprising an Fc domain fused to the C- |
| | terminus of the polypeptide by way of a linker. |
| 93 | The polypeptide of row 92, |
| | wherein the Fc domain comprises or consists of the sequence of |
| | SEQ ID NO: 84 or SEQ ID NO: 79. |
| 94 | The polypeptide of row 93, |
| | wherein the Fc domain comprises or consists of the sequence of |
| | SEQ ID NO: 79. |
| 95 | The polypeptide of any one of rows 1-89, further |
| | comprising an albumin-binding peptide fused |
| | to the C-terminus of the polypeptide by way of a linker. |
| 96 | The polypeptide of row 95, |
| | wherein the albumin-binding peptide comprises the sequence of |
| | SEQ ID NO: 83. |
| 97 | The polypeptide of any one of rows 1-89, further |
| | comprising a fibronectin domain fused to the |
| | C-terminus of the polypeptide by way of a linker. |
| 98 | The polypeptide of row 97, |
| | wherein the fibronectin domain comprises the sequence of SEQ ID |
| | NO: 82. |
| 99 | The polypeptide of any one of rows 1-89, further |
| | comprising a human serum albumin fused to |
| | the C-terminus of the polypeptide by way of a linker. |
| 100 | The polypeptide of row 99, |
| | wherein the human serum albumin comprises the sequence of |
| | SEQ ID NO: 81. |
| 101 | The polypeptide of row 90 or 91, |
| | wherein the polypeptide forms a dimer. |
| 102 | The polypeptide of any one of rows 90-101, |
| | wherein the linker is an amino acid spacer. |
| 103 | The polypeptide of row 102, |
| | wherein the amino acid spacer is GGG, GGGA (SEQ ID NO: 98), |
| | GGGG (SEQ ID NO: 100), GGGAG (SEQ ID NO: 130), GGGAGG (SEQ ID NO: 131), or |
| | GGGAGGG (SEQ ID NO: 132). |
| 104 | The polypeptide of row 103, |
| | wherein the amino acid spacer is GGG. |
| 105 | The polypeptide of row 94 or 104, |
| | wherein the polypeptide has the sequence of SEQ ID NO: |
| | 80. |
| 106 | The polypeptide of row 102, |
| | wherein the amino acid spacer is GGGS (SEQ ID NO: 99), |
| | GGGGA (SEQ ID NO: 101), GGGGS (SEQ ID NO: 102), GGGGG (SEQ ID NO: 103), GGAG |
| | (SEQ ID NO: 104), GGSG (SEQ ID NO: 105), AGGG (SEQ ID NO: 106), SGGG (SEQ ID NO: |
| | 107), GAGA (SEQ ID NO: 108), GSGS (SEQ ID NO: 109), GAGAGA (SEQ ID NO: 110), |
| | GSGSGS (SEQ ID NO: 111), GAGAGAGA (SEQ ID NO: 112), GSGSGSGS (SEQ ID NO: |
| | 113), GAGAGAGAGA (SEQ ID NO: 114), GSGSGSGSGS (SEQ ID NO: 115), |
| | GAGAGAGAGAGA (SEQ ID NO: 116), and GSGSGSGSGSGS (SEQ ID NO: 117), GGAGGA |
| | (SEQ ID NO: 118), GGSGGS (SEQ ID NO: 119), GGAGGAGGA (SEQ ID NO: 120), |
| | GGSGGSGGS (SEQ ID NO: 121), GGAGGAGGAGGA (SEQ ID NO: 122), |
| | GGSGGSGGSGGS (SEQ ID NO: 123), GGAGGGAG (SEQ ID NO: 124), GGSGGGSG (SEQ |
| | ID NO: 125), GGAGGGAGGGAG (SEQ ID NO: 126), and GGSGGGSGGGSG (SEQ ID NO: |
| | 127), GGGGAGGGGAGGGGA (SEQ ID NO: 128), GGGGSGGGGSGGGGS (SEQ ID NO: |
| | 129), AAAL (SEQ ID NO: 133), AAAK (SEQ ID NO: 134), AAAR (SEQ ID NO: 135), |
| | EGKSSGSGSESKST (SEQ ID NO: 136), GSAGSAAGSGEF (SEQ ID NO: 137), |
| | AEAAAKEAAAKA (SEQ ID NO: 96), KESGSVSSEQLAQFRSLD (SEQ ID NO: 95), |
| | GENLYFQSGG (SEQ ID NO: 94), SACYCELS (SEQ ID NO: 93), RSIAT (SEQ ID NO: 92), |
| | RPACKIPNDLKQKVMNH (SEQ ID NO: 91), |
| | GGSAGGSGSGSSGGSSGASGTGTAGGTGSGSGTGSG (SEQ ID NO: 90), |
| | AAANSSIDLISVPVDSR (SEQ ID NO: 89), |
| | GGSGGGSEGGGSEGGGSEGGGSEGGGSEGGGSGGGS (SEQ ID NO: 88), EAAAK (SEQ |
| | ID NO: 87), or PAPAP(SEQ ID NO: 86). |
| 107 | The polypeptide of any one of rows 1-106, |
| | wherein the polypeptide has a serum half-life of at |
| | least 7 days. |
| 108 | The polypeptide of any one of rows 1-107, |
| | wherein the polypeptide binds to activin A, activin |
| | B, and/or myostatin and has reduced or weak binding to human BMP9. |
| 109 | The polypeptide of row 108, |
| | wherein the polypeptide does not substantially bind to human |
| | BMP9. |
| 110 | The polypeptide of any one of rows 1-109, |
| | wherein the polypeptide binds to human activin A |
| | with a K_{D} of 800 pM or less. |
| 111 | The polypeptide of any one of rows 1-110, |
| | wherein the polypeptide binds to human activin B |
| | with a K_{D} of 800 pM or less. |
| 112 | The polypeptide of any one of rows 1-111, |
| | wherein the polypeptide binds to human GDF-11 |
| | with a K_{D} of 5 pM or higher. |
| 113 | A nucleic acid molecule encoding the polypeptide of any one of rows 1-112. |
| 114 | A vector comprising the nucleic acid molecule of row 113. |
| 115 | A host cell that expresses the polypeptide of any one of rows 1-112, |
| | wherein the host cell |
| | comprises the nucleic acid molecule of row 113 or the vector of row 114, |
| | wherein the nucleic |
| | acid molecule or vector is expressed in the host cell. |
| 116 | A pharmaceutical composition comprising the |
| | polypeptide of any one of rows 1-112, the |
| | nucleic acid molecule of row 113, or the vector |
| | of row 114, and one or more pharmaceutically |
| | acceptable carriers or excipients. |
| 117 | The pharmaceutical composition of row 116, |
| | wherein the polypeptide is in a therapeutically |
| | effective amount. |

TABLE 19

| Row | Composition |
|---|---|
| 1 | A polypeptide comprising an extracellular activin receptor type IIB (ActRIIB) variant, the |
| | variant having one or more amino acid substitutions relative to the sequence of |
| | GRGEAETRECIYYNANWELERTNQSGLERCEGEQDKRLHCYASWRNSSGTIELVKKGCWL |
| | DDFNCYDRQECVATEENPQVYFCCCEGNFCNERFTHLPEAGGPEVTYEPPPTAPT (SEQ ID |
| | NO: 74), |
| | wherein the variant comprises one or more amino acid substitutions that impart |
| | reduced BMP9 binding relative to wild type extracellular ActRIIB and one or more additional |
| | amino acid substitutions, |
| | wherein the substitutions that reduce BMP9 binding comprise one or |
| | more of: |
| | a) amino acid substitution E75K; |
| | b) amino acid substitutions Q69T and E70D; or |
| | c) amino acid substitutions Q69D and E70T, |
| | optionally |
| | wherein the variant is truncated from the N-terminus by |
| | deletion of 1, 2, 3, 4, 5, 6, or |
| | 7 amino acids. |
| 2 | The polypeptide of row 1, |
| | wherein the variant comprises one or more amino acid substitutions |
| | selected from the group consisting of 111L, Y12F, L19K, E20D, S25T, L27V, R29P, E31Y, |
| | E33D, Q34K, L38R, Y41F, R45K, S471, S48T, T50S, 151L, L53I, K56Q, F631, T74K, E76D, |
| | N77S, Q79E, and F89M. |
| 3 | The polypeptide of row 1 or 2, |
| | wherein the variant comprises amino acid substitutions E75K, |
| | E20D, and F631. |
| 4 | The polypeptide of row 1 or 2, |
| | wherein the variant comprises amino acid substitution E75K. |
| 5 | The polypeptide of row 4, |
| | wherein the variant comprises amino acid substitutions T74K, E76D, |
| | N77S, and Q79E. |
| 6 | The polypeptide of row 5, |
| | wherein the variant further comprises one or more additional amino |
| | acid substitutions. |
| 7 | The polypeptide of row 6, |
| | wherein the variant comprises amino acid substitutions Y41F, R45K, |
| | and K56Q. |
| 8 | The polypeptide of row 7, |
| | wherein the variant further comprises amino acid substitutions |
| | Y12F, L19K, E20D, R29P, E31Y, E33D, L38R, and F631. |
| 9 | The polypeptide of row 6, |
| | wherein the variant comprises amino acid substitutions S25T and |
| | S471. |
| 10 | The polypeptide of row 9, |
| | wherein the variant comprises amino acid substitution S48T. |
| 11 | The polypeptide of row 6, |
| | wherein the variant comprises amino acid substitution R29P. |
| 12 | The polypeptide of row 6, |
| | wherein the variant comprises amino acid substitutions E31Y, |
| | E33D, and Q34K. |
| 13 | The polypeptide of row 6, |
| | wherein the variant comprises amino acid substitutions Y12F, L19K, |
| | and E20D. |
| 14 | The polypeptide of row 6, |
| | wherein the variant comprises amino acid substitutions E31Y, |
| | E33D, and L38R. |
| 15 | The polypeptide of row 1 or 2, |
| | wherein the variant comprises amino acid substitutions Q69T |
| | and E70D. |
| 16 | The polypeptide of any one of rows 1, 2, and 15, |
| | wherein the variant comprises amino acid |
| | substitutions Q69T and E70D and additional amino acid substitutions 111L, L27V, Q34K, |
| | T50S, 151L, L531, and F89M. |
| 17 | The polypeptide of row 1 or 2, |
| | wherein the variant comprises amino acid substitutions Q69D |
| | and E70T. |
| 18 | The polypeptide of any one of rows 1, 2, and 17, |
| | wherein the variant comprises amino acid |
| | substitutions Q69D and E70T and additional amino acid substitutions 111L, L27V, Q34K, |
| | T50S, 151L, L531, and F89M. |
| 19 | The polypeptide of any one of rows 15-18, |
| | wherein the variant comprises amino acid |
| | substitution E75K. |
| 20 | A polypeptide comprising an ActRIIB variant, the variant having a sequence of |
| | GRGEAETRECX₁X₂YNANWEX₃X₄RTNQX₅GX₆EX-CX₈GX₉X₁₀DKRX₁₁HCX₁₂ASWX₁₃NX₁₄X₁₅ |
| | GX₁₆X₁₇EX₁₈VKX₁₉GCWLDDX₂₀NCYDRX₂₁X₂₂CVAX₂₃X₂₄X₂₅X₂₆PX₂₇VYFCCCEGNX₂₈CNERF |
| | THLPEAGGPEVTYEPPPTAPT (SEQ ID NO: 730), |
| | wherein X₁ is I or L; X₂ is F or Y; X₃ is L or |
| | K; X₄ is D or E; X₅ is T or S; X₆ is L or V; X₇ is P or R; |
| | X₈ is Y or E; X₉ is D or E; X₁₀ is K or Q; |
| | X₁₁ is R or L; X₁₂ is Y or F; X₁₃ is R or K; X₁₄ is S or I; |
| | X₁₅ is S or T; X₁₆ is S or T; X₁₇ is I or L; |
| | X₁₈ is I or L; X₁₉ is K or Q; X₂₀ is F or I; X₂₁ is Q, T, or D; |
| | X₂₂ is E, D, or T; X₂₃ is K or T; X₂₄ is K |
| | or E; X₂₅ is D or E; X₂₆ is S or N; X₂₇ is E or Q; and |
| | X₂₈ is F or M, and |
| | wherein X₂₄ is E and/or |
| | either X₂₁ is T and X₂₂ is D or X₂₁ is D and X₂₂ is T, |
| | and |
| | wherein the variant has at least one |
| | amino acid substitution relative to a wild-type |
| | extracellular ActRIIB having the sequence of |
| | SEQ ID NO: 74, optionally |
| | wherein the variant is |
| | truncated from the N-terminus by deletion of |
| | 1, 2, 3, 4, 5, 6, or 7 amino acids. |
| 21 | The polypeptide of row 20, |
| | wherein X₁ is I. |
| 22 | The polypeptide of row 20, |
| | wherein X₁ is L. |
| 23 | The polypeptide of any one of rows 20-22, |
| | wherein X₂ is F. |
| 24 | The polypeptide of any one of rows 20-22, |
| | wherein X₂ is Y. |
| 25 | The polypeptide of any one of rows 20-24, |
| | wherein X₃ is L. |
| 26 | The polypeptide of any one of rows 20-24, |
| | wherein X₃ is K. |
| 27 | The polypeptide of any one of rows 20-26, |
| | wherein X₄ is D. |
| 28 | The polypeptide of any one of rows 20-26, |
| | wherein X₄ is E. |
| 29 | The polypeptide of any one of rows 20-28, |
| | wherein X₅ is T. |
| 30 | The polypeptide of any one of rows 20-28, |
| | wherein X₅ is S. |
| 31 | The polypeptide of any one of rows 20-30, |
| | wherein X₆ is L. |
| 32 | The polypeptide of any one of rows 20-30, |
| | wherein X₆ is V. |
| 33 | The polypeptide of any one of rows 20-32, |
| | wherein X₇ is P. |
| 34 | The polypeptide of any one of rows 20-32, |
| | wherein X₇ is R. |
| 35 | The polypeptide of any one of rows 20-34, |
| | wherein X₈ is Y. |
| 36 | The polypeptide of any one of rows 20-34, |
| | wherein X₈ is E. |
| 37 | The polypeptide of any one of rows 20-36, |
| | wherein X₉ is D. |
| 38 | The polypeptide of any one of rows 20-36, |
| | wherein X₉ is E. |
| 39 | The polypeptide of any one of rows 20-38, |
| | wherein X₁₀ is K. |
| 40 | The polypeptide of any one of rows 20-38, |
| | wherein X₁₀ is Q. |
| 41 | The polypeptide of any one of rows 20-40, |
| | wherein X₁₁ is R. |
| 42 | The polypeptide of any one of rows 20-40, |
| | wherein X₁₁ is L. |
| 43 | The polypeptide of any one of rows 20-42, |
| | wherein X₁₂ is Y. |
| 44 | The polypeptide of any one of rows 20-42, |
| | wherein X₁₂ is F. |
| 45 | The polypeptide of any one of rows 20-44, |
| | wherein X₁₃ is R. |
| 46 | The polypeptide of any one of rows 20-44, |
| | wherein X₁₃ is K. |
| 47 | The polypeptide of any one of rows 20-46, |
| | wherein X₁₄ is S. |
| 48 | The polypeptide of any one of rows 20-46, |
| | wherein X₁₄ is I. |
| 49 | The polypeptide of any one of rows 20-48, |
| | wherein X₁₅ is S. |
| 50 | The polypeptide of any one of rows 20-48, |
| | wherein X₁₅ is T. |
| 51 | The polypeptide of any one of rows 20-50, |
| | wherein X₁₆ is S. |
| 52 | The polypeptide of any one of rows 20-50, |
| | wherein X₁₆ is T. |
| 53 | The polypeptide of any one of rows 20-52, |
| | wherein X₁₇ is I. |
| 54 | The polypeptide of any one of rows 20-52, |
| | wherein X₁₇ is L. |
| 55 | The polypeptide of any one of rows 20-54, |
| | wherein X₁₈ is I. |
| 56 | The polypeptide of any one of rows 20-54, |
| | wherein X₁₈ is L. |
| 57 | The polypeptide of any one of rows 20-56, |
| | wherein X₁₉ is K. |
| 58 | The polypeptide of any one of rows 20-56, |
| | wherein X₁₉ is Q. |
| 59 | The polypeptide of any one of rows 20-58, |
| | wherein X₂₀ is F. |
| 60 | The polypeptide of any one of rows 20-58, |
| | wherein X₂₀ is I. |
| 61 | The polypeptide of any one of rows 20-60, |
| | wherein X₂₁ is Q. |
| 62 | The polypeptide of any one of rows 20-60, |
| | wherein X₂₁ is T. |
| 63 | The polypeptide of any one of rows 20-60, |
| | wherein X₂₁ is D. |
| 64 | The polypeptide of any one of rows 20-61, |
| | wherein X₂₂ is E. |
| 65 | The polypeptide of any one of rows 20-60 and 62, |
| | wherein X₂₂ is D. |
| 66 | The polypeptide of any one of rows 20-60 and 63, |
| | wherein X₂₂ is T. |
| 67 | The polypeptide of any one of rows 20-66, |
| | wherein X₂₃ is K. |
| 68 | The polypeptide of any one of rows 20-66, |
| | wherein X₂₃ is T. |
| 69 | The polypeptide of any one of rows 20-68, |
| | wherein X₂₄ is K. |
| 70 | The polypeptide of any one of rows 20-60, 62, 63, and 65-68, |
| | wherein X₂₄ is E. |
| 71 | The polypeptide of any one of rows 20-70, |
| | wherein X₂₅ is D. |
| 72 | The polypeptide of any one of rows 20-70, |
| | wherein X₂₅ is E. |
| 73 | The polypeptide of any one of rows 20-72, |
| | wherein X₂₆ is S. |
| 74 | The polypeptide of any one of rows 20-72, |
| | wherein X₂₆ is N. |
| 75 | The polypeptide of any one of rows 20-74, |
| | wherein X₂₇ is E. |
| 76 | The polypeptide of any one of rows 20-74, |
| | wherein X₂₇ is Q. |
| 77 | The polypeptide of any one of rows 20-76, |
| | wherein X₂₈ is F. |
| 78 | The polypeptide of any one of rows 20-76, |
| | wherein X₂₈ is M. |
| 79 | The polypeptide of any one of rows 20-78, |
| | wherein X₂₃ is T, X₂₄ is K, X₂₅ is E, and X₂₆ is N. |
| 80 | The polypeptide of any one of rows 20-78, |
| | wherein X₂₃ is T, X₂₄ is E, X₂₅ is E, and X₂₆ is N. |
| 81 | The polypeptide of any one of rows 20-78, |
| | wherein X₂₃ is K, X₂₄ is K, X₂₅ is D, and X₂₆ is S. |
| 82 | The polypeptide of any one of rows 1-81, |
| | wherein the variant has the sequence of any one of |
| | SEQ ID NOs: 731-744. |
| 83 | The polypeptide of row 82, |
| | wherein the variant has the sequence of SEQ ID NO: 732. |
| 84 | The polypeptide of row 82, |
| | wherein the variant has the sequence of SEQ ID NO: 738. |
| 85 | The polypeptide of row 82, |
| | wherein the variant has the sequence of SEQ ID NO: 741. |
| 86 | The polypeptide of row 82, |
| | wherein the variant has the sequence of SEQ ID NO: 742. |
| 87 | The polypeptide of row 82, |
| | wherein the variant has the sequence of SEQ ID NO: 743. |
| 88 | The polypeptide of row 82, |
| | wherein the variant has the sequence of SEQ ID NO: 744. |
| 89 | The polypeptide of any one of rows 1-88, |
| | wherein the amino acid at position X₂₄ is replaced |
| | with the amino acid K. |
| 90 | The polypeptide of any one of rows 1-88, |
| | wherein the amino acid at position X₂₄ is replaced |
| | with the amino acid E. |
| 91 | The polypeptide of any one of rows 1-90, |
| | wherein the variant is truncated from the N-terminus |
| | by deletion of one amino acid. |
| 92 | The polypeptide of any one of rows 1-90, |
| | wherein the variant is truncated from the N-terminus |
| | by deletion of two amino acids. |
| 93 | The polypeptide of any one of rows 1-90, |
| | wherein the variant is truncated from the N-terminus |
| | by deletion of three amino acids. |
| 94 | The polypeptide of any one of rows 1-90, |
| | wherein the variant is truncated from the N-terminus |
| | by deletion of four amino acids. |
| 95 | The polypeptide of any one of rows 1-90, |
| | wherein the variant is truncated from the N-terminus |
| | by deletion of five amino acids. |
| 96 | The polypeptide of any one of rows 1-90, |
| | wherein the variant is truncated from the N-terminus |
| | by deletion of six amino acids. |
| 97 | The polypeptide of any one of rows 1-90, |
| | wherein the variant is truncated from the N-terminus |
| | by deletion of seven amino acids. |
| 98 | The polypeptide of any one of rows 1-97, further comprising an Fc domain monomer fused to |
| | the C-terminus of the polypeptide by way of a linker. |
| 99 | The polypeptide of row 98, |
| | wherein the Fc domain monomer comprises the sequence of SEQ |
| | ID NO: 97. |
| 100 | The polypeptide of row 98 or 99, |
| | wherein the polypeptide forms a dimer. |
| 101 | The polypeptide of any one of rows 1-97, further comprising an Fc domain fused to |
| | the C-terminus of the polypeptide by way of a linker. |
| 102 | The polypeptide of row 101, |
| | wherein the Fc domain comprises the sequence of SEQ ID NO: |
| | 84 or SEQ ID NO: 79. |
| 103 | The polypeptide of row 102, |
| | wherein the Fc domain comprises the sequence of SEQ ID NO: |
| | 84. |
| 104 | The polypeptide of row 102, |
| | wherein the Fc domain comprises the sequence of SEQ ID NO: |
| | 79. |
| 105 | The polypeptide of any one of rows 1-97, further comprising an albumin- |
| | binding peptide fused to the C-terminus of the polypeptide by way of a linker. |
| 106 | The polypeptide of row 105, |
| | wherein the albumin-binding peptide comprises the sequence of |
| | SEQ ID NO: 83. |
| 107 | The polypeptide of any one of rows 1-97, further comprising a fibronectin |
| | domain fused to the C-terminus of the polypeptide by way of a linker. |
| 108 | The polypeptide of row 107, |
| | wherein the fibronectin domain comprises the sequence of SEQ |
| | ID NO: 82. |
| 109 | The polypeptide of any one of rows 1-97, further comprising a human serum albumin |
| | fused to the C-terminus of the polypeptide by way of a linker. |
| 110 | The polypeptide of row 109, |
| | wherein the human serum albumin comprises the sequence of |
| | SEQ ID NO: 81. |
| 111 | The polypeptide of any one of rows 98-110, |
| | wherein the linker is an amino acid spacer. |
| 112 | The polypeptide of row 111, |
| | wherein the amino acid spacer is GGG, GGGA (SEQ ID NO: 98), |
| | GGGG (SEQ ID NO: 100), GGGAG (SEQ ID NO: 130), GGGAGG (SEQ ID NO: 131), or |
| | GGGAGGG (SEQ ID NO: 132). |
| 113 | The polypeptide of row 112, |
| | wherein the amino acid spacer is GGG. |
| 114 | The polypeptide of row 111, |
| | wherein the amino acid spacer is GA, GS, GG, GGA, GGS, GGG, |
| | GGGS (SEQ ID NO: 99), GGGGA (SEQ ID NO: 101), GGGGS (SEQ ID NO: 102), GGGGG |
| | (SEQ ID NO: 103), GGAG (SEQ ID NO: 104), GGSG (SEQ ID NO: 105), AGGG (SEQ ID NO: |
| | 106), SGGG (SEQ ID NO: 107), GAGA (SEQ ID NO: 108), GSGS (SEQ ID NO: 109), |
| | GAGAGA (SEQ ID NO: 110), GSGSGS (SEQ ID NO: 111), GAGAGAGA (SEQ ID NO: 112), |
| | GSGSGSGS (SEQ ID NO: 113), GAGAGAGAGA (SEQ ID NO: 114), GSGSGSGSGS (SEQ |
| | ID NO: 115), GAGAGAGAGAGA (SEQ ID NO: 116), and GSGSGSGSGSGS (SEQ ID NO: |
| | 117), GGAGGA (SEQ ID NO: 118), GGSGGS (SEQ ID NO: 119), GGAGGAGGA (SEQ ID |
| | NO: 120), GGSGGSGGS (SEQ ID NO: 121), GGAGGAGGAGGA (SEQ ID NO: 122), |
| | GGSGGSGGSGGS (SEQ ID NO: 123), GGAGGGAG (SEQ ID NO: 124), GGSGGGSG (SEQ |
| | ID NO: 125), GGAGGGAGGGAG (SEQ ID NO: 126), and GGSGGGSGGGSG (SEQ ID NO: |
| | 127), GGGGAGGGGAGGGGA (SEQ ID NO: 128), GGGGSGGGGSGGGGS (SEQ ID NO: |
| | 129), AAAL (SEQ ID NO: 133), AAAK (SEQ ID NO: 134), AAAR (SEQ ID NO: 135), |
| | EGKSSGSGSESKST (SEQ ID NO: 136), GSAGSAAGSGEF (SEQ ID NO: 137), |
| | AEAAAKEAAAKA (SEQ ID NO: 96), KESGSVSSEQLAQFRSLD (SEQ ID NO: 95), |
| | GENLYFQSGG (SEQ ID NO: 94), SACYCELS (SEQ ID NO: 93), RSIAT (SEQ ID NO: 92), |
| | RPACKIPNDLKQKVMNH (SEQ ID NO: 91), |
| | GGSAGGSGSGSSGGSSGASGTGTAGGTGSGSGTGSG (SEQ ID NO: 90), |
| | AAANSSIDLISVPVDSR (SEQ ID NO: 89), |
| | GGSGGGSEGGGSEGGGSEGGGSEGGGSEGGGSGGGS (SEQ ID NO: 88), EAAAK (SEQ |
| | ID NO: 87), or PAPAP(SEQ ID NO: 86). |
| 115 | The polypeptide of any one of rows 1-114, |
| | wherein the polypeptide has a serum half-life of at |
| | least 7 days. |
| 116 | The polypeptide of any one of rows 1-115, |
| | wherein the polypeptide binds to activin A, activin |
| | B, and/or myostatin and has reduced or weak binding to human BMP9. |
| 117 | The polypeptide of row 116, |
| | wherein the polypeptide does not substantially bind to human |
| | BMP9. |
| 118 | The polypeptide of any one of rows 1-117, |
| | wherein the polypeptide binds to human activin A |
| | with a K_{D} of 800 pM or less. |
| 119 | The polypeptide of any one of rows 1-118, |
| | wherein the polypeptide binds to human activin B |
| | with a K_{D} of 800 pM or less. |
| 120 | The polypeptide of any one of rows 1-119, |
| | wherein the polypeptide binds to human GDF-11 |
| | with a K_{D} of 5 pM or higher. |
| 121 | A nucleic acid molecule encoding a polypeptide of any one of rows 1-120. |
| 122 | A vector comprising the nucleic acid molecule of row 121. |
| 123 | A host cell that expresses a polypeptide of any one of rows 1-120, |
| | wherein the host cell |
| | comprises a nucleic acid molecule of row 121 or a vector of row 122, |
| | wherein the nucleic acid |
| | molecule or vector is expressed in the host cell. |
| 124 | A pharmaceutical composition comprising a polypeptide of any one of rows 1-120, a nucleic |
| | acid molecule of row 121, or a vector of row 122, and one or more pharmaceutically |
| | acceptable carriers or excipients. |
| 125 | The pharmaceutical composition of row 124, |
| | wherein the polypeptide is in a therapeutically |
| | effective amount. |
| 126 | A construct comprising two identical polypeptides (e.g., a homodimer), each comprising an |
| | extracellular ActRIIB variant of any one of rows 1-97 (e.g., an ActRIIB variant having a |
| | sequence of any one of SEQ ID NOs: 730-744) fused (e.g., linked using an amino acid |
| | spacer) to the N- or C-terminus of an Fc domain monomer (e.g., the sequence of SEQ ID NO: |
| | 97). The two Fc domain monomers in the two polypeptides interact to form an Fc domain in |
| | the construct. |
| 127 | A construct comprising two different polypeptides (e.g., a heterodimer), each comprising an |
| | extracellular ActRIIB variant of any one of rows 1-97 (e.g., an ActRIIB variant having a |
| | sequence of any one of SEQ ID NOs: 730-744) fused (e.g., linked using an amino acid |
| | spacer) to the N- or C-terminus of an Fc domain monomer (e.g., the sequence of SEQ ID NO: |
| | 97). The two Fc domain monomers in the two polypeptides interact to form an Fc domain in |
| | the construct. |

TABLE 20

| Row | Composition |
|---|---|
| 1 | A polypeptide comprising an extracellular activin receptor |
| | type II (ActRII) chimera, the chimera |
| | having a sequence of any one of |
| | GAILGRAETRECIYYNANWELERTNQSGLERCEGEQX₁KRRHCFATWKNISGSIEIVKQGCWLDD |
| | X₂X₃CYDRTDCVX₄X₅X₆X₇X₈PX₉VYFCCCEGNMCNEKFSYFPEMEVTQPTS (SEQ ID NO: 751), |
| | GAILGRAETRECIYYNANWELERTNQSGLERCEGEQX₁KRLHCFATWKNISGSIEIVKQGCWLDD |
| | X₂X₃CYDRTDCVX₄X₅X₆X₇X₈PX₉VYFCCCEGNMCNEKFSYFPEMEVTQPTS (SEQ ID NO: 752), |
| | GAILGRAETRECIYYNANWELERTNQSGLERCEGEQX₁KRLHCYASWKNISGSIEIVKQGCWLDD |
| | X₂X₃CYDRTDCVX₄X₅X₆X₇X₈PX₉VYFCCCEGNMCNEKFSYFPEMEVTQPTS (SEQ ID NO: 753), |
| | GAILGRAETRECIYYNANWELERTNQSGLERCEGEQX₁KRLHCYASWRNSSGSIEIVKQGCWLD |
| | DX₂X₃CYDRTDCVX₄X₅X₆X₇X₈PX₉VYFCCCEGNMCNEKFSYFPEMEVTQPTS (SEQ ID NO: 754), |
| | GAILGRAETRECIYYNANWELERTNQSGLERCEGEQX₁KRLHCYASWRNSSGTIEIVKQGCWLD |
| | DX₂X₃CYDRTDCVX₄X₅X₆X₇X₈PX₉VYFCCCEGNMCNEKFSYFPEMEVTQPTS (SEQ ID NO: 755), |
| | GAILGRAETRECIYYNANWELERTNQSGLERCEGEQX₁KRLHCYASWRNSSGTIELVKKGCWLD |
| | DX₂X₃CYDRTDCVX₄X₅X₆X₇X₈PX₉VYFCCCEGNMCNEKFSYFPEMEVTQPTS (SEQ ID NO: 756), |
| | GAILGRAETRECIYYNANWELERTNQSGLERCEGEQX₁KRLHCYASWRNSSGTIELVKKGCWLD |
| | DX₂X₃CYDRQECVX₄X₅X₆X₇X₈PX₉VYFCCCEGNMCNEKFSYFPEMEVTQPTS (SEQ ID NO: 757), |
| | GRGEAETRECIYYNANWELERTNQSGLERCEGEQX₁KRRHCFATWKNISGSIEIVKQGCWLDDX |
| | 2X₃CYDRTDCVX₄X₅X₆X₇X₈PX₉VYFCCCEGNMCNEKFSYFPEMEVTQPTS (SEQ ID NO: 758), |
| | GRGEAETRECIYYNANWELERTNQSGLERCEGEQX₁KRLHCFATWKNISGSIEIVKQGCWLDDX |
| | 2X₃CYDRTDCVX₄X₅X₆X₇X₈PX₉VYFCCCEGNMCNEKFSYFPEMEVTQPTS (SEQ ID NO: 759), |
| | GRGEAETRECIYYNANWELERTNQSGLERCEGEQX₁KRLHCYASWKNISGSIEIVKQGCWLDDX |
| | 2X₃CYDRTDCVX₄X₅X₆X₇X₈PX₉VYFCCCEGNMCNEKFSYFPEMEVTQPTS (SEQ ID NO: 760), |
| | GRGEAETRECIYYNANWELERTNQSGLERCEGEQX₁KRLHCYASWRNSSGSIEIVKQGCWLDD |
| | X₂X₃CYDRTDCVX₄X₅X₆X₇X₈PX₉VYFCCCEGNMCNEKFSYFPEMEVTQPTS (SEQ ID NO: 761), |
| | GRGEAETRECIYYNANWELERTNQSGLERCEGEQX₁KRLHCYASWRNSSGTIEIVKQGCWLDD |
| | X₂X₃CYDRTDCVX₄X₅X₆X₇X₈PX₉VYFCCCEGNMCNEKFSYFPEMEVTQPTS (SEQ ID NO: 762), |
| | GRGEAETRECIYYNANWELERTNQSGLERCEGEQX₁KRLHCYASWRNSSGTIELVKKGCWLDD |
| | X₂X₃CYDRTDCVX₄X₅X₆X₇X₈PX₉VYFCCCEGNMCNEKFSYFPEMEVTQPTS (SEQ ID NO: 763), |
| | and |
| | GRGEAETRECIYYNANWELERTNQSGLERCEGEQX₁KRLHCYASWRNSSGTIELVKKGCWLDD |
| | X₂X₃CYDRQECVX₄X₅X₆X₇X₈PX₉VYFCCCEGNMCNEKFSYFPEMEVTQPTS (SEQ ID NO: 764), |
| | wherein X₁ is D or R, X₂ is I, F, E, D, Y, S, N, Q, or T, |
| | X₃ is N or T, X₄ is A or E, X₅ is T or K, X₆ is |
| | E or K, X₇ is E or D, X₈ is N or S, and |
| | X₉ is Q, E, K, R, D, or N, optionally |
| | wherein the chimera is |
| | truncated from the N-terminus by deletion |
| | of 1, 2, 3, 4, 5, 6, 7, 8, or 9 amino acids, |
| | wherein the |
| | chimera retains the two amino acids before the first cysteine. |
| 2 | A polypeptide containing an extracellular ActRII chimera, |
| | the chimera having a sequence of any |
| | one of |
| | GAILGRSETQECIYYNANWELERTNQSGLERCEGEQX₁KRRHCFATWKNISGSIEIVKQGCWLD |
| | DX₂X₃CYDRTDCVX₄X₅X₆X₇X₈PX₉VYFCCCEGNMCNEKFSYFPEMEVTQPTS (SEQ ID NO: 765), |
| | GAILGRSETQECIYYNANWELERTNQSGLERCEGEQX₁KRLHCFATWKNISGSIEIVKQGCWLDD |
| | X₂X₃CYDRTDCVX₄X₅X₆X₇X₈PX₉VYFCCCEGNMCNEKFSYFPEMEVTQPTS (SEQ ID NO: 766), |
| | GAILGRSETQECIYYNANWELERTNQSGLERCEGEQX₁KRLHCYASWKNISGSIEIVKQGCWLDD |
| | X₂X₃CYDRTDCVX₄X₅X₆X₇X₈PX₉VYFCCCEGNMCNEKFSYFPEMEVTQPTS (SEQ ID NO: 767), |
| | GAILGRSETQECIYYNANWELERTNQSGLERCEGEQX₁KRLHCYASWRNSSGSIEIVKQGCWLD |
| | DX₂X₃CYDRTDCVX₄X₅X₆X₇X₈PX₉VYFCCCEGNMCNEKFSYFPEMEVTQPTS (SEQ ID NO: 768), |
| | GAILGRSETQECIYYNANWELERTNQSGLERCEGEQX₁KRLHCYASWRNSSGTIEIVKQGCWLD |
| | DX₂X₃CYDRTDCVX₄X₅X₆X₇X₈PX₉VYFCCCEGNMCNEKFSYFPEMEVTQPTS (SEQ ID NO: 769), |
| | GAILGRSETQECIYYNANWELERTNQSGLERCEGEQX₁KRLHCYASWRNSSGTIELVKKGCWLD |
| | DX₂X₃CYDRTDCVX₄X₅X₆X₇X₈PX₉VYFCCCEGNMCNEKFSYFPEMEVTQPTS (SEQ ID NO: 770), |
| | and |
| | GAILGRSETQECIYYNANWELERTNQSGLERCEGEQX₁KRLHCYASWRNSSGTIELVKKGCWLD |
| | DX₂X₃CYDRQECVX₄X₅X₆X₇X₈PX₉VYFCCCEGNMCNEKFSYFPEMEVTQPTS (SEQ ID NO: 771), |
| | in which X₁ is D or R, X₂ is I, F, E, D, Y, S, N, Q, or T, |
| | X₃ is N or T, X₄ is A or E, X₅ is T or K, X₆ is |
| | E or K, X₇ is E or D, X₈ is N or S, and X₉ is Q, E, K, R, D, or N, optionally |
| | wherein the chimera is |
| | truncated from the N-terminus by deletion of |
| | 1, 2, 3, 4, 5, 6, 7, 8, or 9 amino acids, |
| | wherein the |
| | chimera retains the two amino acids before the first cysteine. |
| 3 | The polypeptide of row 1, |
| | wherein the chimera has the sequence of |
| | GAILGRAETRECIYYNANWELERTNQSGLERCEGEQX₁KRRHCFATWKNISGSIEIVKQGCWLDD |
| | X₂X₃CYDRTDCVX₄X₅X₆X₇X₈PX₉VYFCCCEGNMCNEKFSYFPEMEVTQPTS (SEQ ID NO: 751). |
| 4 | The polypeptide of row 1, |
| | wherein the chimera has the sequence of |
| | GAILGRAETRECIYYNANWELERTNQSGLERCEGEQX₁KRLHCFATWKNISGSIEIVKQGCWLDD |
| | X₂X₃CYDRTDCVX₄X₅X₆X₇X₈PX₉VYFCCCEGNMCNEKFSYFPEMEVTQPTS (SEQ ID NO: 752). |
| 5 | The polypeptide of row 1, |
| | wherein the chimera has the sequence of |
| | GAILGRAETRECIYYNANWELERTNQSGLERCEGEQX₁KRLHCYASWKNISGSIEIVKQGCWLDD |
| | X₂X₃CYDRTDCVX₄X₅X₆X₇X₈PX₉VYFCCCEGNMCNEKFSYFPEMEVTQPTS (SEQ ID NO: 753). |
| 6 | The polypeptide of row 1, |
| | wherein the chimera has the sequence of |
| | GAILGRAETRECIYYNANWELERTNQSGLERCEGEQX₁KRLHCYASWRNSSGSIEIVKQGCWLD |
| | DX₂X₃CYDRTDCVX₄X₅X₆X₇X₈PX₉VYFCCCEGNMCNEKFSYFPEMEVTQPTS (SEQ ID NO: 754). |
| 7 | The polypeptide of row 1, |
| | wherein the chimera has the sequence of |
| | GAILGRAETRECIYYNANWELERTNQSGLERCEGEQX₁KRLHCYASWRNSSGTIEIVKQGCWLD |
| | DX₂X₃CYDRTDCVX₄X₅X₆X₇X₈PX₉VYFCCCEGNMCNEKFSYFPEMEVTQPTS (SEQ ID NO: 755). |
| 8 | The polypeptide of row 1, |
| | wherein the chimera has the sequence of |
| | GAILGRAETRECIYYNANWELERTNQSGLERCEGEQX₁KRLHCYASWRNSSGTIELVKKGCWLD |
| | DX₂X₃CYDRTDCVX₄X₅X₆X₇X₈PX₉VYFCCCEGNMCNEKFSYFPEMEVTQPTS (SEQ ID NO: 756). |
| 9 | The polypeptide of row 1, |
| | wherein the chimera has the sequence of |
| | GAILGRAETRECIYYNANWELERTNQSGLERCEGEQX₁KRLHCYASWRNSSGTIELVKKGCWLD |
| | DX₂X₃CYDRQECVX₄X₅X₆X₇X₈PX₉VYFCCCEGNMCNEKFSYFPEMEVTQPTS (SEQ ID NO: 757). |
| 10 | The polypeptide of row 1, |
| | wherein the chimera has the sequence of |
| | GRGEAETRECIYYNANWELERTNQSGLERCEGEQX₁KRRHCFATWKNISGSIEIVKQGCWLDDX |
| | 2X₃CYDRTDCVX₄X₅X₆X₇X₈PX₉VYFCCCEGNMCNEKFSYFPEMEVTQPTS (SEQ ID NO: 758). |
| 11 | The polypeptide of row 1, |
| | wherein the chimera has the sequence of |
| | GRGEAETRECIYYNANWELERTNQSGLERCEGEQX₁KRLHCFATWKNISGSIEIVKQGCWLDDX |
| | 2X₃CYDRTDCVX₄X₅X₆X₇X₈PX₉VYFCCCEGNMCNEKFSYFPEMEVTQPTS (SEQ ID NO: 759). |
| 12 | The polypeptide of row 1, |
| | wherein the chimera has the sequence of |
| | GRGEAETRECIYYNANWELERTNQSGLERCEGEQX₁KRLHCYASWKNISGSIEIVKQGCWLDDX |
| | 2X₃CYDRTDCVX₄X₅X₆X₇X₈PX₉VYFCCCEGNMCNEKFSYFPEMEVTQPTS (SEQ ID NO: 760). |
| 13 | The polypeptide of row 1, |
| | wherein the chimera has the sequence of |
| | GRGEAETRECIYYNANWELERTNQSGLERCEGEQX₁KRLHCYASWRNSSGSIEIVKQGCWLDD |
| | X₂X₃CYDRTDCVX₄X₅X₆X₇X₈PX₉VYFCCCEGNMCNEKFSYFPEMEVTQPTS (SEQ ID NO: 761). |
| 14 | The polypeptide of row 1, |
| | wherein the chimera has the sequence of |
| | GRGEAETRECIYYNANWELERTNQSGLERCEGEQX₁KRLHCYASWRNSSGTIEIVKQGCWLDD |
| | X₂X₃CYDRTDCVX₄X₅X₆X₇X₈PX₉VYFCCCEGNMCNEKFSYFPEMEVTQPTS (SEQ ID NO: 762). |
| 15 | The polypeptide of row 1, |
| | wherein the chimera has the sequence of |
| | GRGEAETRECIYYNANWELERTNQSGLERCEGEQX₁KRLHCYASWRNSSGTIELVKKGCWLDD |
| | X₂X₃CYDRTDCVX₄X₅X₆X₇X₈PX₉VYFCCCEGNMCNEKFSYFPEMEVTQPTS (SEQ ID NO: 763). |
| 16 | The polypeptide of row 1, |
| | wherein the chimera has the sequence of |
| | GRGEAETRECIYYNANWELERTNQSGLERCEGEQX₁KRLHCYASWRNSSGTIELVKKGCWLDD |
| | X₂X₃CYDRQECVX₄X₅X₆X₇X₈PX₉VYFCCCEGNMCNEKFSYFPEMEVTQPTS (SEQ ID NO: 764). |
| 17 | The polypeptide of row 2, |
| | wherein the chimera has the sequence of |
| | GAILGRSETQECIYYNANWELERTNQSGLERCEGEQX₁KRRHCFATWKNISGSIEIVKQGCWLD |
| | DX₂X₃CYDRTDCVX₄X₅X₆X₇X₈PX₉VYFCCCEGNMCNEKFSYFPEMEVTQPTS (SEQ ID NO: 765). |
| 18 | The polypeptide of row 2, |
| | wherein the chimera has the sequence of |
| | GAILGRSETQECIYYNANWELERTNQSGLERCEGEQX₁KRLHCFATWKNISGSIEIVKQGCWLDD |
| | X₂X₃CYDRTDCVX₄X₅X₆X₇X₈PX₉VYFCCCEGNMCNEKFSYFPEMEVTQPTS (SEQ ID NO: 766). |
| 19 | The polypeptide of row 2, |
| | wherein the chimera has the sequence of |
| | GAILGRSETQECIYYNANWELERTNQSGLERCEGEQX₁KRLHCYASWKNISGSIEIVKQGCWLDD |
| | X₂X₃CYDRTDCVX₄X₅X₆X₇X₈PX₉VYFCCCEGNMCNEKFSYFPEMEVTQPTS (SEQ ID NO: 767). |
| 20 | The polypeptide of row 2, |
| | wherein the chimera has the sequence of |
| | GAILGRSETQECIYYNANWELERTNQSGLERCEGEQX₁KRLHCYASWRNSSGSIEIVKQGCWLD |
| | DX₂X₃CYDRTDCVX₄X₅X₆X₇X₈PX₉VYFCCCEGNMCNEKFSYFPEMEVTQPTS (SEQ ID NO: 768). |
| 21 | The polypeptide of row 2, |
| | wherein the chimera has the sequence of |
| | GAILGRSETQECIYYNANWELERTNQSGLERCEGEQX₁KRLHCYASWRNSSGTIEIVKQGCWLD |
| | DX₂X₃CYDRTDCVX₄X₅X₆X₇X₈PX₉VYFCCCEGNMCNEKFSYFPEMEVTQPTS (SEQ ID NO: 769). |
| 22 | The polypeptide of row 2, |
| | wherein the chimera has the sequence of |
| | GAILGRSETQECIYYNANWELERTNQSGLERCEGEQX₁KRLHCYASWRNSSGTIELVKKGCWLD |
| | DX₂X₃CYDRTDCVX₄X₅X₆X₇X₈PX₉VYFCCCEGNMCNEKFSYFPEMEVTQPTS (SEQ ID NO: 770). |
| 23 | The polypeptide of row 2, |
| | wherein the chimera has the sequence of |
| | GAILGRSETQECIYYNANWELERTNQSGLERCEGEQX₁KRLHCYASWRNSSGTIELVKKGCWLD |
| | DX₂X₃CYDRQECVX₄X₅X₆X₇X₈PX₉VYFCCCEGNMCNEKFSYFPEMEVTQPTS (SEQ ID NO: 771). |
| 24 | The polypeptide of any one of rows 1-23, |
| | wherein X₁ is D. |
| 25 | The polypeptide of any one of rows 1-23, |
| | wherein X₁ is R. |
| 26 | The polypeptide of any one of rows 1-25, |
| | wherein X₂ is I. |
| 27 | The polypeptide of any one of rows 1-25, |
| | wherein X₂ is F. |
| 28 | The polypeptide of any one of rows 1-25, |
| | wherein X₂ is E. |
| 29 | The polypeptide of any one of rows 1-25, |
| | wherein X₂ is D. |
| 30 | The polypeptide of any one of rows 1-25, |
| | wherein X₂ is Y. |
| 31 | The polypeptide of any one of rows 1-25, |
| | wherein X₂ is S. |
| 32 | The polypeptide of any one of rows 1-25, |
| | wherein X₂ is N. |
| 33 | The polypeptide of any one of rows 1-25, |
| | wherein X₂ is Q. |
| 34 | The polypeptide of any one of rows 1-25, |
| | wherein X₂ is T. |
| 35 | The polypeptide of any one of rows 1-34, |
| | wherein X₃ is N. |
| 36 | The polypeptide of any one of rows 1-34, |
| | wherein X₃ is T. |
| 37 | The polypeptide of any one of rows 1-36, |
| | wherein X₄ is A. |
| 38 | The polypeptide of any one of rows 1-36, |
| | wherein X₄ is E. |
| 39 | The polypeptide of any one of rows 1-38, |
| | wherein X₅ is T. |
| 40 | The polypeptide of any one of rows 1-38, |
| | wherein X₅ is K. |
| 41 | The polypeptide of any one of rows 1-40, |
| | wherein X₆ is E. |
| 42 | The polypeptide of any one of rows 1-40, |
| | wherein X₆ is K. |
| 43 | The polypeptide of any one of rows 1-42, |
| | wherein X₇ is E. |
| 44 | The polypeptide of any one of rows 1-42, |
| | wherein X₇ is D. |
| 45 | The polypeptide of any one of rows 1-44, |
| | wherein X₈ is N. |
| 46 | The polypeptide of any one of rows 1-44, |
| | wherein X₈ is S. |
| 47 | The polypeptide of any one of rows 1-46, |
| | wherein X₉ is Q. |
| 48 | The polypeptide of any one of rows 1-46, |
| | wherein X₉ is E. |
| 49 | The polypeptide of any one of rows 1-46, |
| | wherein X₉ is K. |
| 50 | The polypeptide of any one of rows 1-46, |
| | wherein X₉ is R. |
| 51 | The polypeptide of any one of rows 1-46, |
| | wherein X₉ is D. |
| 52 | The polypeptide of any one of rows 1-46, |
| | wherein X₉ is N. |
| 53 | The polypeptide of any one of rows 1-52, |
| | wherein X₅ is T, X₆ is E, X₇ is E, and X₈ is N. |
| 54 | The polypeptide of any one of rows 1-52, |
| | wherein X₅ is T, X₆ is K, X₇ is E, and X₈ is N. |
| 55 | The polypeptide of any one of rows 1-54, |
| | wherein X₂ is E and X₃ is T. |
| 56 | The polypeptide of any one of rows 1-54, |
| | wherein X₂ is I or F and X₃ is N. |
| 57 | The polypeptide of row 56, |
| | wherein X₂ is I. |
| 58 | The polypeptide of row 56, |
| | wherein X₂ is F. |
| 59 | The polypeptide of row 1 or row 2, |
| | wherein the chimera has the sequence of any one of SEQ ID |
| | NOs: 772-793. |
| 60 | The polypeptide of row 59, |
| | wherein the chimera has the sequence of SEQ ID NO: 772. |
| 61 | The polypeptide of row 59, |
| | wherein the chimera has the sequence of SEQ ID NO: 773. |
| 62 | The polypeptide of row 59, |
| | wherein the chimera has the sequence of SEQ ID NO: 774. |
| 63 | The polypeptide of row 59, |
| | wherein the chimera has the sequence of SEQ ID NO: 775. |
| 64 | The polypeptide of row 59, |
| | wherein the chimera has the sequence of SEQ ID NO: 790. |
| 65 | The polypeptide of row 59, |
| | wherein the chimera has the sequence of SEQ ID NO: 791. |
| 66 | The polypeptide of row 59, |
| | wherein the chimera has the sequence of SEQ ID NO: 778. |
| 67 | The polypeptide of row 59, |
| | wherein the chimera has the sequence of SEQ ID NO: 792. |
| 68 | The polypeptide of row 59, |
| | wherein the chimera has the sequence of SEQ ID NO: 793. |
| 69 | The polypeptide of row 59, |
| | wherein the chimera has the sequence of SEQ ID NO: 787. |
| 70 | The polypeptide of any one of rows 1-69, |
| | wherein the chimera is truncated from the N-terminus |
| | by deletion of one amino acid. |
| 71 | The polypeptide of any one of rows 1-69, |
| | wherein the chimera is truncated from the N-terminus |
| | by deletion of two amino acids. |
| 72 | The polypeptide of any one of rows 1-69, |
| | wherein the chimera is truncated from the N-terminus |
| | by deletion of three amino acids. |
| 73 | The polypeptide of any one of rows 1-69, |
| | wherein the chimera is truncated from the N-terminus |
| | by deletion of four amino acids. |
| 74 | The polypeptide of any one of rows 1-69, |
| | wherein the chimera is truncated from the N-terminus |
| | by deletion of five amino acids. |
| 75 | The polypeptide of any one of rows 1-69, |
| | wherein the chimera is truncated from the N-terminus |
| | by deletion of six amino acids. |
| 76 | The polypeptide of any one of rows 1-69, |
| | wherein the chimera is truncated from the N-terminus |
| | by deletion of seven amino acids. |
| 77 | The polypeptide of any one of rows 1-69, |
| | wherein the chimera is truncated from the N-terminus |
| | by deletion of eight amino acids. |
| 78 | The polypeptide of any one of rows 1-69, |
| | wherein the chimera is truncated from the N-terminus |
| | by deletion of nine amino acids. |
| 79 | The polypeptide of any one of rows 1-78, |
| | wherein the polypeptide (e.g., the chimera) further |
| | includes a C-terminal extension of one or more amino |
| | acids (e.g., 1, 2, 3, 4, 5, 6, or more amino |
| | acids from wild-type extracellular ActRIIA or ActRIIB). |
| 80 | The polypeptide of row 79, |
| | wherein the C-terminal extension is NP. |
| 81 | The polypeptide of row 79, |
| | wherein the C-terminal extension is NPVTPK (SEQ ID NO: 78). |
| 82 | The polypeptide of any one of rows 1-81, |
| | wherein the polypeptide further includes an Fc domain |
| | monomer fused to the C-terminus of the polypeptide |
| | (e.g., the C-terminus of the chimera) by way |
| | of a linker. |
| 83 | The polypeptide of row 82, |
| | wherein the Fc domain monomer has the sequence of SEQ ID NO: |
| | 97. |
| 84 | The polypeptide of row 82 or 83, |
| | wherein the polypeptide forms a dimer. |
| 85 | The polypeptide of any one of rows 1-81, |
| | wherein the polypeptide further includes an Fc domain |
| | fused to the C-terminus of the polypeptide |
| | (e.g., the C-terminus of the chimera) by way of a linker. |
| 86 | The polypeptide of row 85, |
| | wherein the Fc domain comprises the sequence of SEQ ID NO: 84 or |
| | SEQ ID NO: 79. |
| 87 | The polypeptide of any one of rows 1-81, |
| | wherein the polypeptide further includes an albumin- |
| | binding peptide fused to the C-terminus of the |
| | polypeptide (e.g., the C-terminus of the chimera) |
| | by way of a linker. |
| 88 | The polypeptide of row 87, |
| | wherein the albumin-binding peptide has the sequence of SEQ ID NO: |
| | 83. |
| 89 | The polypeptide of any one of rows 1-81, |
| | wherein the polypeptide further includes a fibronectin |
| | domain fused to the C-terminus of the polypeptide |
| | (e.g., the C-terminus of the chimera) by way of |
| | a linker. |
| 90 | The polypeptide of row 89, |
| | wherein the fibronectin domain has the sequence of SEQ ID NO: 82. |
| 91 | The polypeptide of any one of rows 1-81, |
| | wherein the polypeptide further includes a human |
| | serum albumin fused to the C-terminus of the polypeptide |
| | (e.g., the C-terminus of the chimera) by |
| | way of a linker. |
| 92 | The polypeptide of row 91, |
| | wherein the human serum albumin has the sequence of SEQ ID NO: |
| | 81. |
| 93 | The polypeptide of any one of rows 82-92, |
| | wherein the linker is an amino acid spacer. |
| 94 | The polypeptide of row 93, |
| | wherein the amino acid spacer is GGG, GGGA (SEQ ID NO: 98), |
| | GGGG (SEQ ID NO: 100), GGGAG (SEQ ID NO: 130), GGGAGG (SEQ ID NO: 131), or |
| | GGGAGGG (SEQ ID NO: 132). |
| 95 | The polypeptide of row 93, |
| | wherein the amino acid spacer is GGS, GGGS (SEQ ID NO: 99), |
| 96 | GGGGS (SEQ ID NO: 102), GGSG (SEQ ID NO: 105), or SGGG (SEQ ID NO: 107). |
| | The polypeptide of row 93, |
| | wherein the amino acid spacer is GA, GS, GG, GGA, GGS, GGG, |
| | GGGS (SEQ ID NO: 99), GGGGA (SEQ ID NO: 101), GGGGS (SEQ ID NO: 102), GGGGG |
| | (SEQ ID NO: 103), GGAG (SEQ ID NO: 104), GGSG (SEQ ID NO: 105), AGGG (SEQ ID NO: |
| | 106), SGGG (SEQ ID NO: 107), GAGA (SEQ ID NO: 108), GSGS (SEQ ID NO: 109), GAGAGA |
| | (SEQ ID NO: 110), GSGSGS (SEQ ID NO: 111), GAGAGAGA (SEQ ID NO: 112), GSGSGSGS |
| | (SEQ ID NO: 113), GAGAGAGAGA (SEQ ID NO: 114), GSGSGSGSGS (SEQ ID NO: 115), |
| | GAGAGAGAGAGA (SEQ ID NO: 116), and GSGSGSGSGSGS (SEQ ID NO: 117), GGAGGA |
| | (SEQ ID NO: 118), GGSGGS (SEQ ID NO: 119), GGAGGAGGA (SEQ ID NO: 120), |
| | GGSGGSGGS (SEQ ID NO: 121), GGAGGAGGAGGA (SEQ ID NO: 122), GGSGGSGGSGGS |
| | (SEQ ID NO: 123), GGAGGGAG (SEQ ID NO: 124), GGSGGGSG (SEQ ID NO: 125), |
| | GGAGGGAGGGAG (SEQ ID NO: 126), and GGSGGGSGGGSG (SEQ ID NO: 127), |
| | GGGGAGGGGAGGGGA (SEQ ID NO: 128), GGGGSGGGGSGGGGS (SEQ ID NO: 129), AAAL |
| | (SEQ ID NO: 133), AAAK (SEQ ID NO: 134), AAAR (SEQ ID NO: 135), EGKSSGSGSESKST |
| | (SEQ ID NO: 136), GSAGSAAGSGEF (SEQ ID NO: 137), AEAAAKEAAAKA (SEQ ID NO: 96), |
| | KESGSVSSEQLAQFRSLD (SEQ ID NO: 95), GENLYFQSGG (SEQ ID NO: 94), SACYCELS |
| | (SEQ ID NO: 93), RSIAT (SEQ ID NO: 92), RPACKIPNDLKQKVMNH (SEQ ID NO: 91), |
| | GGSAGGSGSGSSGGSSGASGTGTAGGTGSGSGTGSG (SEQ ID NO: 90), |
| | AAANSSIDLISVPVDSR (SEQ ID NO: 89), |
| | GGSGGGSEGGGSEGGGSEGGGSEGGGSEGGGSGGGS (SEQ ID NO: 88), EAAAK (SEQ ID |
| | NO: 87), or PAPAP(SEQ ID NO: 86). |
| 97 | The polypeptide of any one of rows 1-96, |
| | wherein the polypeptide (e.g., an ActRII chimera-Fc |
| | fusion protein) has a serum half-life of at least seven days. |
| 98 | The polypeptide of any one of rows 1-97, |
| | wherein the polypeptide has increased binding to one or |
| | more an ActRII ligands (e.g., activin A, activin B, |
| | myostatin, and/or GDF-11) compared to wild- |
| | type ActRIIA and/or wild-type ActRIIB (e.g., wild-type |
| | extracellular ActRIIA and/or ActRIIB). |
| 99 | The polypeptide of any one of rows 1-98, |
| | wherein the polypeptide has decreased binding to bone |
| | morphogenetic protein 9 (BMP9, e.g., human BMP9) |
| | compared to wild-type ActRIIB (e.g., wild- |
| | type extracellular ActRIIB). |
| 100 | The polypeptide of any one of rows 1-99, |
| | wherein the polypeptide binds to activin A, activin B, |
| | and/or myostatin and has reduced or weak binding to |
| | human BMP9 (e.g., compared to wild-type |
| | extracellular ActRIIB). |
| 101 | The polypeptide of any one of rows 1-100, |
| | wherein the polypeptide does not substantially bind to |
| | human BMP9. |
| 102 | The polypeptide of any one of rows 1-101, |
| | wherein the polypeptide binds to human activin A with |
| | a K_{D} of 800 pM or less. |
| 103 | The polypeptide of any one of rows 1-102, |
| | wherein the polypeptide binds to human activin B with |
| | a K_{D} of 800 pM or less. |
| 104 | The polypeptide of any one of rows 1-103, |
| | wherein the polypeptide binds to human GDF-11 with |
| | a K_{D} of 5 pM or higher. |
| 105 | A nucleic acid molecule encoding the polypeptide of any one of rows 1-104. |
| 106 | A vector comprising the nucleic acid molecule of row 105. |
| 107 | A host cell that expresses the polypeptide of any one of rows 1-104, |
| | wherein the host cell |
| | comprises the nucleic acid molecule of row 105 or the vector of row 106, |
| | wherein the nucleic acid |
| | molecule or vector is expressed in the host cell. |
| 108 | A pharmaceutical composition comprising the |
| | polypeptide of any one of rows 1-104, the nucleic |
| | acid molecule of row 105, or the vector of |
| | row 106 and one or more pharmaceutically acceptable |
| | carriers or excipients. |
| 109 | The pharmaceutical composition of row 108, |
| | wherein the polypeptide, nucleic acid molecule, or |
| | vector is in a therapeutically effective amount. |
| 110 | A construct comprising two identical polypeptides |
| | (e.g., a homodimer), each comprising an |
| | extracellular ActRII chimera of any one of rows |
| | 1-81 fused (e.g., linked using an amino acid |
| | spacer) to the N- or C-terminus of an Fc domain |
| | monomer (e.g., the sequence of SEQ ID NO: |
| | 97). The two Fc domain monomers in the two polypeptides |
| | interact to form an Fc domain in the |
| | construct. |
| 111 | A construct comprising two different polypeptides |
| | (e.g., a heterodimer), each comprising an |
| | extracellular ActRII chimera of any one of rows |
| | 1-81 fused (e.g., linked using an amino acid |
| | spacer) to the N- or C-terminus of an Fc domain |
| | monomer (e.g., the sequence of SEQ ID NO: |
| | 97). The two Fc domain monomers in the two polypeptides |
| | interact to form an Fc domain in the |
| | construct. |

TABLE 21

| Row | Composition |
|---|---|
| 1 | A polypeptide comprising an extracellular activin receptor |
| | type II (ActRII) chimera, the chimera |
| | having a sequence of X₁β₁X₂β₂X₃β₃X₄β₄X₅β₅X₆β₆X₇β₇X₈, |
| | wherein: |
| | X₁ is GAILGRSETQ (SEQ ID NO: 794) or GRGEAETR (SEQ ID NO: 795); |
| | β₁ is ECLFFN (β₁ₐ) (SEQ ID NO: 796) or ECIYYN (β_{1b}) (SEQ ID NO: 797); |
| | X₂ is ANWEKDRTN (SEQ ID NO: 798) or ANWELERTN (SEQ ID NO: 799); |
| | β₂ is QTGVEPC (β₂ₐ) (SEQ ID NO: 800) or QSGLERC (β_{2b}) (SEQ ID NO: 801); |
| | X₃ is YGDKDKR (SEQ ID NO: 802) or EGEQDKR (SEQ ID NO: 803); |
| | β₃ is RHCFATWKNI (β₃ₐ) (SEQ ID NO: 804) or a portion |
| | thereof that comprises HCFATWK (SEQ |
| | ID NO: 805) or LHCYASWRNS (β_{3b}) (SEQ ID NO: 806) |
| | or a portion thereof that comprises |
| | HCYASWR (SEQ ID NO: 807), |
| | wherein when β₃ is HCFATWK or HCYASWR, the chimera |
| | comprises contiguous amino acids from ActRIIA or |
| | ActRIIB connecting β₃ to X₃ and X₄; |
| | X₄ is SG; |
| | β₄ is SIEIVKQGCW (β₄ₐ) (SEQ ID NO: 808) or a portion |
| | thereof that comprises EIVKQGCW (SEQ |
| | ID NO: 809) or TIELVKKGCW (β_{4b}) (SEQ ID NO: 810) or |
| | a portion thereof that comprises |
| | ELVKKGCW (SEQ ID NO: 811), |
| | wherein when β₄ is EIVKQGCW or ELVKKGCW, the chimera |
| | comprises contiguous amino acids from ActRIIA or ActRIIB connecting β₄ to X₄; |
| | X₅ is LDDINCYDRTDC (SEQ ID NO: 812) or LDDENCYDRQEC (SEQ ID NO: 813); |
| | β₅ is VEK (β₅ₐ) or a portion thereof that comprises |
| | VE or VAT (β_{5b}) or a portion thereof that |
| | comprises V, |
| | wherein when β₅ is VE or V, the chimera comprises |
| | contiguous amino acids from |
| | ActRIIA or ActRIIB connecting β₅ to X₆; |
| | X₆ is KDSPEV (SEQ ID NO: 814) or EENPQV (SEQ ID NO: 815); |
| | β₆ is YFCCCE (SEQ ID NO: 816); |
| | X₇ is GNMCNE (SEQ ID NO: 817) or GNFCNE (SEQ ID NO: 818); |
| | β₇ is KFSYF (β₇ₐ) (SEQ ID NO: 819) or a portion |
| | thereof that comprises SYF or RFTHL (β_{7b}) (SEQ |
| | ID NO: 820) or a portion thereof that comprises T, |
| | wherein when β₇ is SYF or T, the chimera |
| | comprises contiguous amino acids from ActRIIA or |
| | ActRIIB connecting β₇ to X₇ and X₈; and |
| | X₈ is PEMEVTQPTS (SEQ ID NO: 821) or PEAGGPEVTYEPPPTAPT (SEQ ID NO: 822), |
| | wherein at least one of β₁ₐ, β₂ₐ, β₃ₐ, β₄ₐ, β₅ₐ, or β₇ₐ and |
| | at least one of β_{1b}, β_{2b}, β_{3b}, β_{4b}, β_{5b}, or β_{7b} |
| | is present in the chimera, optionally |
| | wherein the chimera is truncated from the N-terminus by |
| | deletion of 1, 2, 3, 4, 5, 6, 7, 8, or 9 amino acids, |
| | wherein the chimera retains the two amino acids |
| | before the first cysteine. |
| 2 | The polypeptide of row 1, |
| | wherein X₁ is GAILGRSETQ. |
| 3 | The polypeptide of row 1, |
| | wherein X₁ is GRGEAETR. |
| 4 | The polypeptide of any one of rows 1-3, |
| | wherein β₁ is ECLFFN (β₁ₐ). |
| 5 | The polypeptide of any one of rows 1-3, |
| | wherein β₁ is ECIYYN (β_{1b}). |
| 6 | The polypeptide of any one of rows 1-5, |
| | wherein X₂ is ANWEKDRTN. |
| 7 | The polypeptide of any one of row s 1-5, |
| | wherein X₂ is ANWELERTN. |
| 8 | The polypeptide of any one of rows 1-7, |
| | wherein β₂ is QTGVEPC (β₂ₐ). |
| 9 | The polypeptide of any one of rows 1-7, |
| | wherein β₂ is QSGLERC (β_{2b}). |
| 10 | The polypeptide of any one of rows 1-9, |
| | wherein X₃ is YGDKDKR. |
| 11 | The polypeptide of any one of rows 1-9, |
| | wherein X₃ is EGEQDKR. |
| 12 | The polypeptide of any one of rows 1-11, |
| | wherein β₃ is RHCFATWKNI (β₃ₐ). |
| 13 | The polypeptide of any one of rows 1-11, |
| | wherein β₃ is LHCYASWRNS (β_{3b}). |
| 14 | The polypeptide of any one of rows 1-11, |
| | wherein β₃ is HCFATWK, |
| | wherein the chimera |
| | comprises contiguous amino acids from ActRIIA |
| | or ActRIIB connecting β₃ to X₃ and X₄ |
| 15 | The polypeptide of any one of rows 1-11, |
| | wherein β₃ is HCYASWR, |
| | wherein the chimera |
| | comprises contiguous amino acids from ActRIIA |
| | or ActRIIB connecting β₃ to X₃ and X₄. |
| 16 | The polypeptide of row 14 or 15, |
| | wherein the contiguous amino acids connecting β₃ to X₃ are from |
| | ActRIIA. |
| 17 | The polypeptide of row 14 or 15, |
| | wherein the contiguous amino acids connecting β₃ to X₃ are from |
| | ActRIIB. |
| 18 | The polypeptide of any one of rows 14-17, |
| | wherein the contiguous amino acids connecting β₃ to |
| | X₄ are from ActRIIA. |
| 19 | The polypeptide of any one of rows 14-17, |
| | wherein the contiguous amino acids connecting β₃ to |
| | X₄ are from ActRIIB. |
| 20 | The polypeptide of any one of rows 1-19, |
| | wherein β₄ is SIEIVKQGCW (β₄ₐ). |
| 21 | The polypeptide of any one of rows 1-19, |
| | wherein β₄ is TIELVKKGCW (β_{4b}). |
| 22 | The polypeptide of any one of rows 1-19, |
| | wherein β₄ is EIVKQGCW, |
| | wherein the chimera |
| | comprises contiguous amino acids from ActRIIA or ActRIIB connecting β₄ to X₄. |
| 23 | The polypeptide of any one of rows 1-19, |
| | wherein β₄ is ELVKKGCW, |
| | wherein the chimera |
| | comprises contiguous amino acids from ActRIIA or ActRIIB connecting β₄ to X₄. |
| 24 | The polypeptide of row 22 or 23, |
| | wherein the contiguous amino acids connecting β₄ to X₄ are from |
| | ActRIIA. |
| 25 | The polypeptide of row 22 or 23, |
| | wherein the contiguous amino acids connecting β₄ to X₄ are from |
| | ActRIIB. |
| 26 | The polypeptide of any one of rows 1-25, |
| | wherein X₅ is LDDINCYDRTDC. |
| 27 | The polypeptide of any one of rows 1-25, |
| | wherein X₅ is LDDENCYDRQEC. |
| 28 | The polypeptide of any one of rows 1-27, |
| | wherein β₅ is VEK (β₅ₐ). |
| 29 | The polypeptide of any one of rows 1-27, |
| | wherein β₅ is VAT (β_{5b}). |
| 30 | The polypeptide of any one of rows 1-27, |
| | wherein β₅ is VE, |
| | wherein the chimera comprises |
| | contiguous amino acids from ActRIIA or ActRIIB connecting β₅ to X₆. |
| 31 | The polypeptide of any one of rows 1-27, |
| | wherein β₅ is V, |
| | wherein the chimera comprises |
| | contiguous amino acids from ActRIIA or ActRIIB connecting β₅ to X₆. |
| 32 | The polypeptide of row 30 or 31, |
| | wherein the contiguous amino acids connecting β₅ to X₆ are from |
| | ActRIIA. |
| 33 | The polypeptide of row 30 or 31, |
| | wherein the contiguous amino acids connecting β₅ to X₆ are from |
| | ActRIIB. |
| 34 | The polypeptide of any one of rows 1-33, |
| | wherein X₆ is KDSPEV. |
| 35 | The polypeptide of any one of rows 1-33, |
| | wherein X₆ is EENPQV. |
| 36 | The polypeptide of any one of rows 1-35, |
| | wherein X₇ is GNMCNE. |
| 37 | The polypeptide of any one of rows 1-35, |
| | wherein X₇ is GNFCNE. |
| 38 | The polypeptide of any one of rows 1-37, |
| | wherein β₇ is KFSYF (β₇ₐ). |
| 39 | The polypeptide of any one of rows 1-37, |
| | wherein β₇ is RFTHL (β_{7b}). |
| 40 | The polypeptide of any one of rows 1-37, |
| | wherein β₇ is SYF, |
| | wherein the chimera comprises |
| | contiguous amino acids from ActRIIA or ActRIIB connecting β₇ to X₇. |
| 41 | The polypeptide of any one of rows 1-37, |
| | wherein β₇ is T, |
| | wherein the chimera comprises |
| | contiguous amino acids from ActRIIA or ActRIIB connecting β₇ to X₇ and X₈. |
| 42 | The polypeptide of row 40 or 41, |
| | wherein the contiguous amino acids connecting β₇ to X₇ are from |
| | ActRIIA. |
| 43 | The polypeptide of row 40 or 41, |
| | wherein the contiguous amino acids connecting β₇ to X₇ are from |
| | ActRIIB. |
| 44 | The polypeptide of any one of rows 41-43, |
| | wherein the contiguous amino acids connecting β₇ to |
| | X₈ are from ActRIIA. |
| 45 | The polypeptide of any one of rows 41-43, |
| | wherein the contiguous amino acids connecting β₇ to |
| | X₈ are from ActRIIB. |
| 46 | The polypeptide of any one of rows 1-45, |
| | wherein X₈ is PEMEVTQPTS. |
| 47 | The polypeptide of any one of rows 1-45, |
| | wherein X₈ is PEAGGPEVTYEPPPTAPT. |
| 48 | The polypeptide of any one of rows 1-47, |
| | wherein the chimera is truncated from the N-terminus by |
| | deletion of one amino acid. |
| 49 | The polypeptide of any one of rows 1-47, |
| | wherein the chimera is truncated from the N-terminus by |
| | deletion of two amino acids. |
| 50 | The polypeptide of any one of rows 1-47, |
| | wherein the chimera is truncated from the N-terminus by |
| | deletion of three amino acids. |
| 51 | The polypeptide of any one of rows 1-47, |
| | wherein the chimera is truncated from the N-terminus by |
| | deletion of four amino acids. |
| 52 | The polypeptide of any one of rows 1-47, |
| | wherein the chimera is truncated from the N-terminus by |
| | deletion of five amino acids. |
| 53 | The polypeptide of any one of rows 1-47, |
| | wherein the chimera is truncated from the N-terminus by |
| | deletion of six amino acids. |
| 54 | The polypeptide of any one of rows 1-47, |
| | wherein the chimera is truncated from the N-terminus by |
| | deletion of seven amino acids. |
| 55 | The polypeptide of any one of rows 1-47, |
| | wherein the chimera is truncated from the N-terminus by |
| | deletion of eight amino acids |
| 56 | The polypeptide of any one of rows 1-47, |
| | wherein the chimera is truncated from the N-terminus by |
| | deletion of nine amino acids. |
| 57 | The polypeptide of any one of rows 1-56, further |
| | comprising a C-terminal extension of one or more |
| | amino acids. |
| 58 | The polypeptide of row 57, |
| | wherein the C-terminal extension is NP. |
| 59 | The polypeptide of row 57, |
| | wherein the C-terminal extension is NPVTPK (SEQ ID NO: 78). |
| 60 | The polypeptide of any one of rows 1-59, |
| | wherein the polypeptide further includes an Fc domain |
| | monomer fused to the C-terminus of the polypeptide |
| | (e.g., the C-terminus of the chimera) by way |
| | of a linker. |
| 61 | The polypeptide of row 60, |
| | wherein the Fc domain monomer comprises the sequence of SEQ ID |
| | NO: 97. |
| 62 | The polypeptide of row 60 or 61, |
| | wherein the polypeptide forms a dimer. |
| 63 | The polypeptide of any one of rows 1-59, |
| | wherein the polypeptide further includes an Fc domain |
| | fused to the C-terminus of the polypeptide (e.g., |
| | the C-terminus of the chimera) by way of a linker. |
| 64 | The polypeptide of row 63, |
| | wherein the Fc domain comprises the sequence of SEQ ID NO: 84 or |
| | SEQ ID NO: 79. |
| 65 | The polypeptide of any one of rows 1-59, |
| | wherein the polypeptide further includes an albumin- |
| | binding peptide fused to the C-terminus of the polypeptide |
| | (e.g., the C-terminus of the chimera) by |
| | way of a linker. |
| 66 | The polypeptide of row 65, |
| | wherein the albumin-binding peptide has the sequence of SEQ ID NO: |
| | 83. |
| 67 | The polypeptide of any one of rows 1-59, |
| | wherein the polypeptide further includes a fibronectin |
| | domain fused to the C-terminus of the polypeptide |
| | (e.g., the C-terminus of the chimera) by way of |
| | a linker. |
| 68 | The polypeptide of row 67, |
| | wherein the fibronectin domain has the sequence of SEQ ID NO: 82. |
| 69 | The polypeptide of any one of rows 1-59, |
| | wherein the polypeptide further includes a human serum |
| | albumin fused to the C-terminus of the polypeptide |
| | (e.g., the C-terminus of the chimera) by way of |
| | a linker. |
| 70 | The polypeptide of row 69, |
| | wherein the human serum albumin has the sequence of SEQ ID NO: |
| | 81. |
| 71 | The polypeptide of any one of rows 60-70, |
| | wherein the linker is an amino acid spacer. |
| 72 | The polypeptide of row 71, |
| | wherein the amino acid spacer is GGG, GGGA (SEQ ID NO: 98), |
| | GGGG (SEQ ID NO: 100), GGGAG (SEQ ID NO: 130), |
| | GGGAGG (SEQ ID NO: 131), or |
| | GGGAGGG (SEQ ID NO: 132). |
| 73 | The polypeptide of row 71, |
| | wherein the amino acid spacer is GGS, GGGS (SEQ ID NO: 99), |
| | GGGGS (SEQ ID NO: 102), GGSG (SEQ ID NO: 105), or SGGG (SEQ ID NO: 107). |
| 74 | The polypeptide of row 71, |
| | wherein the amino acid spacer is GA, GS, GG, GGA, GGS, GGG, |
| | GGGS (SEQ ID NO: 99), GGGGA (SEQ ID NO: 101), GGGGS (SEQ ID NO: 102), GGGGG (SEQ |
| | ID NO: 103), GGAG (SEQ ID NO: 104), GGSG (SEQ ID NO: 105), AGGG (SEQ ID NO: 106), |
| | SGGG (SEQ ID NO: 107), GAGA (SEQ ID NO: 108), GSGS (SEQ ID NO: 109), GAGAGA (SEQ |
| | ID NO: 110), GSGSGS (SEQ ID NO: 111), GAGAGAGA (SEQ ID NO: 112), GSGSGSGS (SEQ |
| | ID NO: 113), GAGAGAGAGA (SEQ ID NO: 114), GSGSGSGSGS (SEQ ID NO: 115), |
| | GAGAGAGAGAGA (SEQ ID NO: 116), and GSGSGSGSGSGS (SEQ ID NO: 117), GGAGGA |
| | (SEQ ID NO: 118), GGSGGS (SEQ ID NO: 119), GGAGGAGGA (SEQ ID NO: 120), |
| | GGSGGSGGS (SEQ ID NO: 121), GGAGGAGGAGGA (SEQ ID NO: 122), GGSGGSGGSGGS |
| | (SEQ ID NO: 123), GGAGGGAG (SEQ ID NO: 124), GGSGGGSG (SEQ ID NO: 125), |
| | GGAGGGAGGGAG (SEQ ID NO: 126), and GGSGGGSGGGSG (SEQ ID NO: 127), |
| | GGGGAGGGGAGGGGA (SEQ ID NO: 128), GGGGSGGGGSGGGGS (SEQ ID NO: 129), AAAL |
| | (SEQ ID NO: 133), AAAK (SEQ ID NO: 134), AAAR (SEQ ID NO: 135), EGKSSGSGSESKST |
| | (SEQ ID NO: 136), GSAGSAAGSGEF (SEQ ID NO: 137), AEAAAKEAAAKA (SEQ ID NO: 96), |
| | KESGSVSSEQLAQFRSLD (SEQ ID NO: 95), GENLYFQSGG (SEQ ID NO: 94), SACYCELS |
| | (SEQ ID NO: 93), RSIAT (SEQ ID NO: 92), RPACKIPNDLKQKVMNH (SEQ ID NO: 91), |
| | GGSAGGSGSGSSGGSSGASGTGTAGGTGSGSGTGSG (SEQ ID NO: 90), |
| | AAANSSIDLISVPVDSR (SEQ ID NO: 89), |
| | GGSGGGSEGGGSEGGGSEGGGSEGGGSEGGGSGGGS (SEQ ID NO: 88), EAAAK (SEQ ID |
| | NO: 87), or PAPAP(SEQ ID NO: 86). |
| 75 | The polypeptide of any one of rows 1-74, |
| | wherein the polypeptide (e.g., an ActRII chimera-Fc |
| | fusion protein) has a serum half-life of at least seven days. |
| 76 | The polypeptide of any one of rows 1-75, |
| | wherein the polypeptide binds to activin A, activin B, |
| | and/or myostatin and has reduced or weak binding to |
| | human BMP9 (e.g., compared to wild-type |
| | extracellular ActRIIB). |
| 77 | The polypeptide of any one of rows 1-76, |
| | wherein the polypeptide does not substantially bind to |
| | human BMP9. |
| 78 | The polypeptide of any one of rows 1-77, |
| | wherein the polypeptide binds to human activin A with a |
| | K_{D} of 800 pM or less. |
| 79 | The polypeptide of any one of rows 1-78, |
| | wherein the polypeptide binds to human activin B with a |
| | K_{D} of 800 pM or less. |
| 80 | The polypeptide of any one of rows 1-79, |
| | wherein the polypeptide binds to human GDF-11 with a |
| | K_{D} of 5 pM or higher. |
| 81 | A nucleic acid molecule encoding the polypeptide of any one of rows 1-80. |
| 82 | A vector comprising the nucleic acid molecule of row 81. |
| 83 | A host cell that expresses the polypeptide of any one of rows 1-80, |
| | wherein the host cell |
| | comprises the nucleic acid molecule of row 81 or the vector of row 82, |
| | wherein the nucleic acid |
| | molecule or vector is expressed in the host cell. |
| 84 | A pharmaceutical composition comprising the |
| | polypeptide of any one of rows 1-80, the nucleic |
| | acid molecule of row 81, or the vector of |
| | row 82 and one or more pharmaceutically acceptable |
| | carriers or excipients. |
| 85 | The pharmaceutical composition of row 84, |
| | wherein the polypeptide, nucleic acid molecule, or |
| | vector is in a therapeutically effective amount. |

### EXAMPLES

The following examples are provided to further illustrate some embodiments of the present invention, but are not intended to limit the scope of the invention; it will be understood by their exemplary nature that other procedures, methodologies, or techniques known to those skilled in the art may alternatively be used.

### Example 1—Effect of ActRIIA/B-hFc on Anemia in Patients with Very Low, Low, or Intermediate Risk Myelodysplastic Syndromes

Patients with IPSS-R very-low, low, or intermediate risk MDS and anemia were enrolled in a multi-center, open-label, two-part, Phase 2 study. Participants received doses of 0.75 mg/kg (cohort 1), 1.5 mg/kg (cohort 2), 2.5 mg/kg (cohort 3), 3.75 mg/kg (cohort 4), or 5.0 mg/kg (cohort 5) ActRIIA/B-hFc (SEQ ID NO: 80), administered subcutaneously once every 4 weeks for 12 weeks. In Part 1, dose escalation, ActRIIA/B-hFc is being evaluated in cohorts consisting of 6 participants with anemia (defined as hemoglobin <10 g/dL or requiring red blood cell transfusions), at each dose level stratified 1:1 into participants with ring sideroblasts (RS+) and participants who are non-RS. Safety endpoints include incidence of adverse events (AEs) and progression to higher-risk MDS/AML. Efficacy endpoints include a hemoglobin increase of ≥1.5 g/dL for at least 8 weeks in patients with a low-transfusion burden, a reduction of ≥4 U or ≥50% units transfused over at least 8 weeks compared to baseline in patients with a high transfusion burden, and transfusion independence for at least 8 weeks in participants with either a low transfusion burden (1-3 RBC units in 8 weeks prior to treatment with ActRIIA/B-hFc) or a high transfusion burden. Among the first 12 participants enrolled in the study, 50% were RS+ and non-RS, 50% had erythropoietin >100 mlU/mL, 50% had a high transfusion burden (≥4 U/8 weeks), and 85% had multilineage dysplasia, and participants had a mean platelet count of 192.4×10⁹/L. Patients did not receive prior treatment with azacitidine, decitabine, lenalidomide, luspatercept or sotatercept. Both ESA naïve and experienced patients were eligible.

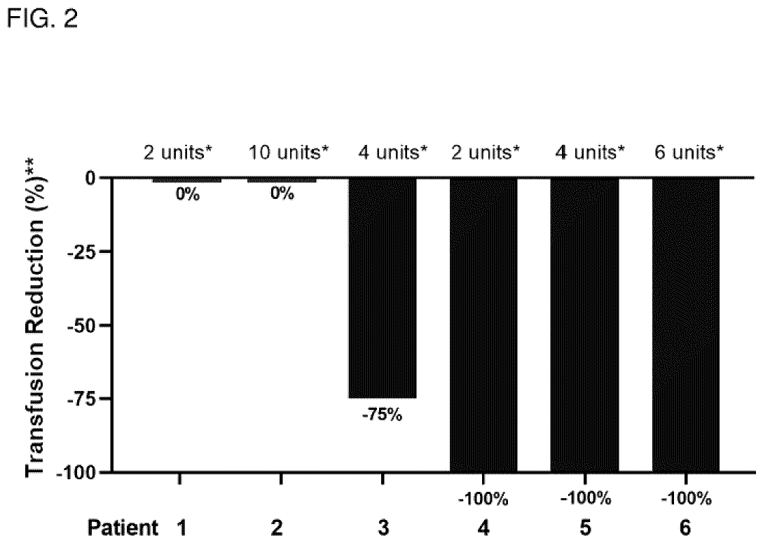

After completing eight weeks of treatment with ActRIIA/B-hFc, no drug-related serious adverse events were observed and increases in reticulocytes, hemoglobin, and platelets were observed in both RS positive and non-RS patients that required transfusions at baseline (≥2 red blood cell units over 8 weeks). Five patients that completed eight weeks of treatment met at least one of the following endpoints: increase in hemoglobin ≥1.5 g/dL for 8 weeks, 50% reduction in transfusion requirements over 8 weeks, or transfusion independence for at least 8 weeks, with three patients achieving transfusion independence ≥8 weeks in duration, two of whom were RS positive and one of whom was non-RS. As shown in FIG. 2, clinically meaningful reductions in transfusion burden as well as transfusion independence were observed with a Q4W dosing schedule. The reduction in transfusions was observed in both RS positive and non-RS patients.

### Example 2—Effect of ActRIIA/B-hFc on Anemia in Patients with Very Low, Low, or Intermediate Risk Myelodysplastic Syndromes

A second assessment was performed on the subjects from the study described in Example 1 once additional participants had been enrolled and study duration had increased. At the time of the second assessment with median follow-up of 140 days (range 1 to 169 days), 17 participants had received ≥1 dose of ActRIIA/B-hFc across three dose levels: 0.75 mg/kg, 1.5 mg/kg, and 2.5 mg/kg. Baseline characteristics for the 17 participants are described in Table 22, below. Results from this assessment are reported for efficacy-evaluable participants in cohorts 1 and 2 of Part 1, dose escalation, defined as having ≥8 weeks of hemoglobin and transfusion data (10 patients). These ten patients had completed at least 8 weeks of treatment with ActRIIA/B-hFc. Of the ten evaluable patients, three were non-transfused (NT, subjects who received 0 RBC units in the 8 weeks prior to treatment with ActRIIA/B-hFc and had a hemoglobin level of <10 g/dL), two were transfused subjects with a low transfusion burden (LTB, subjects who received 1-3 RBC units in the 8 weeks prior to treatment with ActRIIA/B-hFc and had a hemoglobin level of <10 g/dL), and five were transfused subjects with a high transfusion burden (HTB, subjects who received 4 or more RBC units in the 8 weeks prior to treatment with ActRIIA/B-hFc). Of the seven transfused subjects with a LTB or HTB, three did not have ring sideroblasts (non-RS) and four had ring sideroblasts (RS+).

ActRIIA/B-hFc was well tolerated in Cohorts 1 and 2. No drug-related serious adverse events (SAEs), dose-limiting toxicities, or dose modifications were reported. Additionally, no patients developed high risk MDS or AML. There were four treatment-emergent SAEs reported in three patients, all of which were deemed unrelated to study drug, including anemia, febrile illness, pneumonia, and death. Two patients discontinued study drug prior to completing 8 weeks of treatment with ActRIIA/B-hFc, one due to death deemed unrelated to study drug and one patient withdrew consent. There was one observed treatment-related adverse event of maculopapular rash that was moderate in severity (grade 2). The rash was reported after the patient's first dose and resolved without recurrence following subsequent doses.

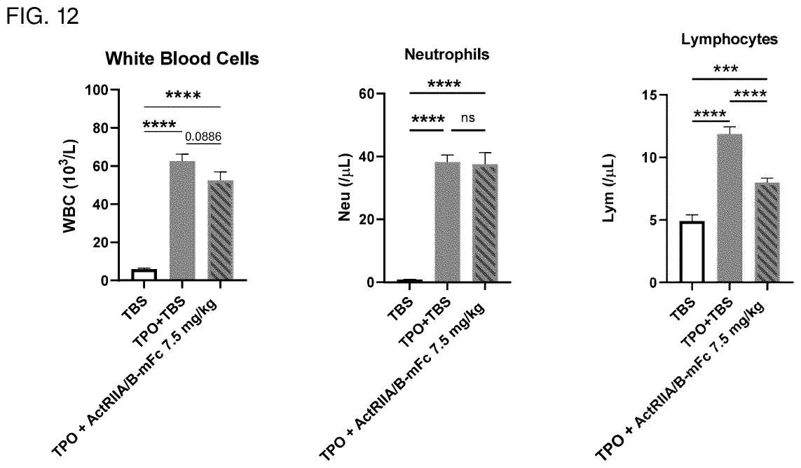

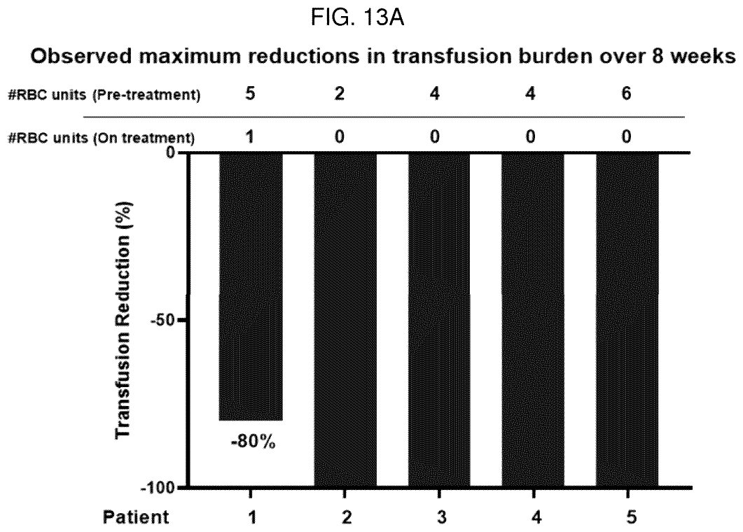

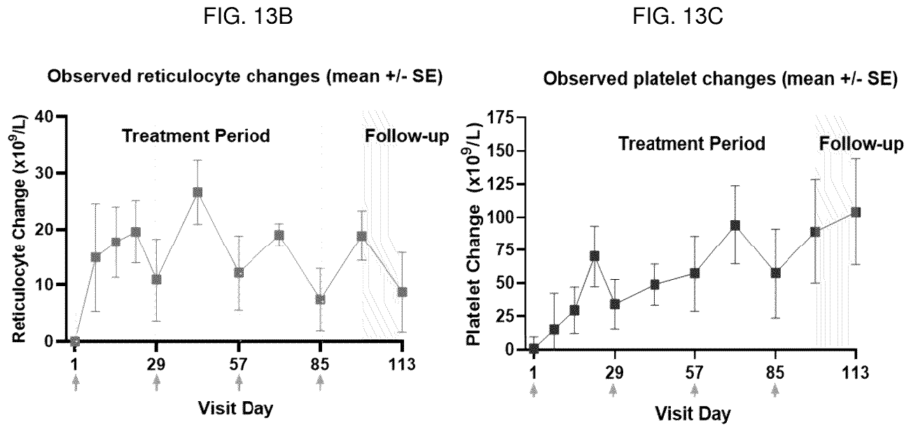

In 10 efficacy-evaluable participants, the overall erythroid response rate was 60% (n=6/10), as six of the 10 patients met at least one of the following endpoints: increase in hemoglobin ≥1.5 g/dL for 8 weeks, 50% reduction in transfusion requirements over 8 weeks, or transfusion independence for at least 8 weeks. Thirty-three percent (n=⅓) of participants who did not require an RBC transfusion in the 8 weeks prior to treatment with ActRIIA/B-hFc (non-transfused participants) had a hemoglobin increase of ≥1.5 g/dL sustained for ≥8 weeks. Five of seven transfused evaluable participants (71%) (n=½ transfused with a low transfusion burden (LTB) and n=⅘ transfused with a high transfusion burden (HTB); n=⅔ non-RS and n=¾ RS+) had erythroid responses sustained for ≥8 weeks (range 8-20 weeks, ongoing) and had at least a 50% reduction in transfusion requirements over 8 weeks. These patients are referred to as the transfused responders (TR). In addition, 57% (n= 4/7) of the transfused evaluable patients achieved transfusion independence for at least 8 weeks (FIG. 13A).

The observed maximum increase from baseline in reticulocytes observed in transfused responders (TR) (n=5) was 24.6×109/L (mean), range 10.5-41.6×109/L from day 1-29 with increases in reticulocytes observed after each dose (FIG. 13B). The observed maximum reduction in serum ferritin in TR was 40.4% (mean), range 10-66%, and the observed maximum increase in soluble transferrin receptor (sTfR) in TR was 52.8% (mean), range 29.8-116.4%.

TABLE 22
Demographics and Baseline Characteristics

| | Cohort 1 | | Cohort 2 | | Cohort 3 | | | |
|---|---|---|---|---|---|---|---|---|
| | 0.75 mg/kg | | 1.5 mg/kg | | 2.5 mg/kg | | Total | |
| Parameter | N = 6 | | N = 6 | | N = 5 | | N = 17 | |
| Age (years), Mean (range) | 75.5 | (70-88) | 68.3 | (55-81) | 70.4 | (64-80) | 71.5 | (55-88) |
| Gender | | | | | | | | |
| Female | 5 | (83.3%) | 1 | (16.7%) | 3 | (60%) | 9 | (52.9%) |
| Male | 1 | (16.7%) | 5 | (83.3%) | 2 | (40%) | 8 | (47.1%) |
| Years since MDS Diagnosis, Mean | 2.84 | (0.4-8.7) | 2.32 | (0.3-3.4) | 1.05 | (0.5-1.6) | 2.37 | (0.3-8.7) |
| (range) | | | | | | | | |
| Ring sideroblast status, n (%) | | | | | | | | |
| RS positive | 3 | (50%) | 3 | (50%) | 4 | (80%) | 10 | (58.8%) |
| Non-RS | 3 | (50%) | 3 | (50%) | 1 | (20%) | 7 | (41.2%) |
| WHO MDS classification, n (%) | | | | | | | | |
| MDS-MLD | 3 | (50%) | 3 | (50%) | 2 | (40%) | 8 | (47.1%) |
| MDS-RS-MLD | 2 | (33.3%) | 2 | (33.3%) | 3 | (60%) | 7 | (41.2%) |
| MDS-RS-SLD | 0 | (0%) | 1 | (16.7%) | 0 | (0%) | 1 | (5.9%) |
| MDS with isolated del 5(q) | 1 | (16.7%) | 0 | (0%) | 0 | (0%) | 1 | (5.9%) |
| Prior ESA Therapy, n (%) | 0 | (0%) | 0 | (0%) | 2 | (40%) | 2 | (11.8%) |
| Iron chelator therapy (%) | 0 | (0%) | 2 | (33.3%) | 2 | (40%) | 4 | (23.5%) |
| RBC transfusion status, | | | | | | | | |
| units per 8 week, n (%) | | | | | | | | |
| Non-transfused (NT), 0 units | 3 | (50%) | 0 | (0%) | 2 | (40%) | 5 | (29.4%) |
| Transfused (LTB and HTB) | 3 | (50%) | 6 | (100%) | 3 | (60%) | 12 | (70.6%) |
| Low transfusion burden (LTB) <4 | 2 | (33.3%) | 0 | (0%) | 1 | (20%) | 3 | (17.6%) |
| units | | | | | | | | |
| High transfusion burden (HTB), ≥4 | 1 | (16.7%) | 6 | (100%) | 2 | (40%) | 9 | (52.9%) |
| units | | | | | | | | |
| Efficacy Evaluable n (%) | 6/6 | (100%) | 4/6 | (66.6%) | 0/5 | (0%) | 10/17 | (58.8%) |

Increases in platelets were observed in TR (FIG. 13C). The mean baseline platelet count for TR was 234×10⁹/L (range 104-401×10⁹/L), and the maximum increase from baseline was 130×109/L (mean), range 32-235×10⁹/L. No participants required dose reduction due to thrombocytosis.

In summary, erythroid responses have been observed in RS+ and non-RS MDS patients including a reduction in transfusion burden at initial dose levels. The observed increases in reticulocytes and sTfR and observed decreases in serum ferritin suggest that administration of ActRIIA/B-hFc is potentially associated with increased erythropoiesis. Increases in platelets have been observed in TR. These data support the potential of ActRIIA/B-hFc as a treatment for multilineage cytopenias in MDS by potentially targeting multiple stages of hematopoiesis.

### Example 3—Effect of ActRIIA/B-hFc on Anemia in Patients with Very Low, Low, or Intermediate Risk Myelodysplastic Syndromes

A third assessment was performed on the subjects from the study described in Example 1 once additional participants had been enrolled and study duration had increased. Baseline characteristics for the 24 participants enrolled in the study are provided in Table 23, below. An overview of baseline characteristics of RS+ and non-RS patients is provided in Table 24. At baseline, non-RS patients had lower reticulocyte and platelet counts, higher endogenous EPO levels, and lower sTfR than RS+ patients, suggesting a greater degree of ineffective hematopoiesis. The majority of patients enrolled ( 19/24, 79%) required transfusions at baseline and ( 20/24, 83%) had multilineage dysplasia. Patients were efficacy evaluable if they had completed at least eight weeks of treatment as of the third assessment date.

TABLE 23
Baseline Characteristics

| | ActRIIA/B-hFc Dose Level (mg/kg) | | | | | |
|---|---|---|---|---|---|---|
| | 0.75 | 1.5 | 2.5 | 3.75 | All | |
| | (N = 6) | (N = 6) | (N = 6) | (N = 6) | (N = 24) | |
| Age, mean (range) | 75.5 | 68.3 | 72.0 | 73.3 | 72.3 | (55-88) |
| Male, n (%) | 1 | 5 | 2 | 4 | 12 | (50%) |
| Years since MDS diagnosis, mean (range) | 2.7 | 2.2 | 0.9 | 2.7 | 2.2 | (0.2-8.6) |
| WHO Disease Category, n (%) | | | | | | |
| MDS-MLD | 3 | 3 | 3 | 1 | 10 | (42%) |
| MDS-RS-MLD | 2 | 2 | 3 | 3 | 10 | (42%) |
| MDS-RS-SLD | 0 | 1 | 0 | 0 | 1 | (4%) |
| MDS with isolated del(5q) | 1 | 0 | 0 | 0 | 1 | (4%) |
| N/A | 0 | 0 | 0 | 2 | 2 | (8%) |
| Transfusion Burden | | | | | | |
| NT | 3 | 0 | 1 | 1 | 5 | (21%) |
| LTB | 2 | 0 | 1 | 2 | 5 | (21%) |
| HTB | 1 | 6 | 4 | 3 | 14 | (58%) |
| RS Status (RS+) | 3 | 3 | 3 | 4 | 13 | (54%) |
| Prior ESA Treatment, n (%) | 0 | 0 | 2 | 1 | 3 | (13%) |
| Current Iron Chelator, n (%) | 0 | 2 | 2 | 2 | 6 | (25%) |
| Efficacy Evaluable (8-week endpoints), n (%) | 6 | 4* | 6 | 0 | 16 | (67%) |

*two participants in Cohort 2 (1.5 mg/kg) were not efficacy-evaluable due to withdrawal of consent (n = 1) and death (n = 1) and six participants in Cohort 4 (3.75 mg/kg) were not efficacy evaluable as they had not completed 8 weeks on study by the time of the third assessment.

TABLE 24
Baseline Characteristics by RS Status

| | RS Status | | |
|---|---|---|---|
| | Mean (SD) | | |
| | | RS+ | Non-RS |
| Biomarker | | (N = 13) | (N = 11) |

| | | | | |
|---|---|---|---|---|
| EPO (IU/L) | 326.0 | (826.2) | 490.2 | (855.7) |
| Reticulocytes (10⁹/L) | 39.1 | (28.0) | 29.8 | (24.8) |
| Platelets (10⁹/L) | 228.4 | (67.8) | 134.6 | (55.5) |
| TPO (pg/mL) | 74.2 | (69.3) | 42.8 | (60.1) |
| sTfR (mg/L) | 1.9 | (1.0) | 1.2 | (0.6) |

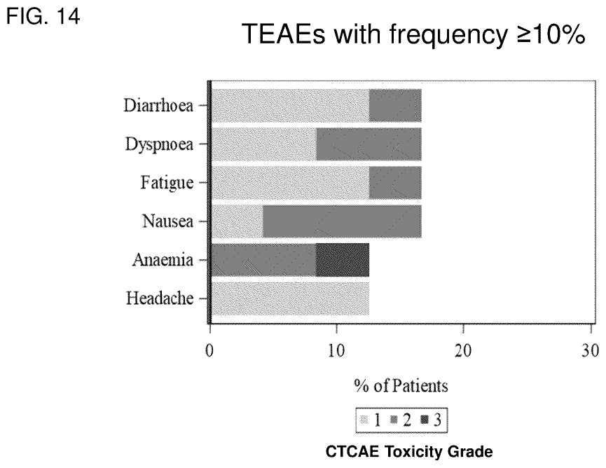

As of the third assessment date, 24 patients in Cohorts 1, 2, 3, and 4 had received at least one dose of ActRIIA/B-hFc. ActRIIA/B-hFc was generally well-tolerated with no dose-dependent findings as of the third assessment date. No drug-related serious adverse events or dose-limiting toxicities were reported. The most commonly reported treatment-emergent adverse events were nausea, fatigue, diarrhea and dyspnea, none of which were deemed related to study drug. Treatment-related adverse events were reported in four patients, which were mild or moderate in severity, and did not lead to dose modification or treatment discontinuation. The treatment-related AE of maculopapular rash was reported in one patient, after the patient's first dose, and resolved without recurrence following subsequent doses. No patients developed high-risk MDS or acute myeloid leukemia. Two patients in Cohort 2 withdrew from the trial prior to completing eight weeks of treatment with ActRIIA/B-hFc, one due to death deemed unrelated to study drug and one due to withdrawn patient consent. The few related AEs are listed below in Table 25, and there were no serious treatment-related AEs and/or treatment-related AEs grade 3 or higher. None of the serious TEAEs were deemed related to study drug. Treatment-emergent AEs of >10% frequency are listed in FIG. 14. There was one death on study in Cohort 2 which was deemed to be not related to study drug.

TABLE 25
Number and Percentage of Patients with Treatment Emergent Adverse
Event Relative to Safety Population as of Third Assessment Date

| | ActRIIA/B-hFc Dose Level (mg/kg) | | | | | | | |
|---|---|---|---|---|---|---|---|---|
| | 0.75 | | 1.5 | | 2.5 | 3.75 | All | |
| n (%) | (N = 6) | | (N = 6) | | (N = 6) | (N = 6) | (N = 24) | |
| Any TEAE | 6 | (100) | 6 | (100) | 6 (100) | 3 (50.0) | 21 | (87.5) |
| Treatment-related AE* | 1 | (16.7) | 0 | | 2 (33.3) | 1 (16.7) | 4 | (16.7) |
| Grade ≥3 TEAE | 1 | (16.7) | 1 | (16.7) | 2 (33.3) | 1 (16.7) | 5 | (20.8) |
| Any serious TEAE** | 1 | (16.7) | 2 | (33.3) | 0 | 2 (33.3) | 5 | (20.8) |
| Any TEAE requiring | 0 | | 0 | | 0 | 1 (16.7) | 1 | (4.2) |
| dose modification | | | | | | | | |

| | | | | | | | |
|---|---|---|---|---|---|---|---|
| Death | 0 | 1 | (16.7) | 0 | 0 | 1 | (4.2) |

*Treatment-related AEs with maximum grade: Grade 1 (Headache, Pain in extremity, Abdominal pain), Grade 2 (Rash, Diarrhea, Nausea, Peripheral edema).
**Serious TEAEs with maximum grade: Grade 2 (Pyrexia, Cardiac failure congestive), Grade 3 (Anaemia, Pneumonia, Pneumothorax), Grade 5 (Death - obesity related heart disease), all deemed unrelated to study drug.

Sixteen patients in Cohorts 1, 2 and 3 had completed at least eight weeks of treatment and evaluation as of the data cut-off date (“evaluable patients”). Of the 16 evaluable patients, four were non-transfused (NT, subjects who received 0 RBC units in the 8 weeks prior to treatment with ActRIIA/B-hFc and had a hemoglobin level of <10 g/dL), three were transfused subjects with a low transfusion burden (LTB, subjects who received 1-3 RBC units in the 8 weeks prior to treatment with ActRIIA/B-hFc and had a hemoglobin level of <10 g/dL), and nine were transfused subjects with a high transfusion burden (HTB, subjects who received 4 or more RBC units in the 8 weeks prior to treatment with ActRIIA/B-hFc). Of the 12 transfused subjects with a LTB or HTB, six did not have ring sideroblasts (non-RS) and six had ring sideroblasts (RS+). Of the 16 evaluable patients, 8 were non-RS and 8 were RS+. Two patients in Cohort 2 were not efficacy-evaluable due to withdrawal of consent (n=1) and death (n=1). Six patients in Cohort 4 were not efficacy-evaluable as they had not completed 8 weeks of evaluation and treatment as of the third assessment date. As of the third assessment date, 50% (n= 8/16) of the evaluable patients, three of whom were non-RS and five of whom were RS+, achieved an overall erythroid response, which is defined as meeting one of the following two endpoints:
IWG 2006 Hematological improvement-erythroid (HI-E), which is defined as either:
   a ≥1.5 g/dL increase in hemoglobin for eight weeks in LTB and NT patients; or
   a reduction by ≥4 red blood cell (RBC) units transfused during any eight-week period during the trial, compared with the eight-week period prior to Cycle 1, Day 1 in HTB patients.
Transfusion independence (TI) for at least eight weeks in patients who required ≥2 RBC units transfused at baseline.

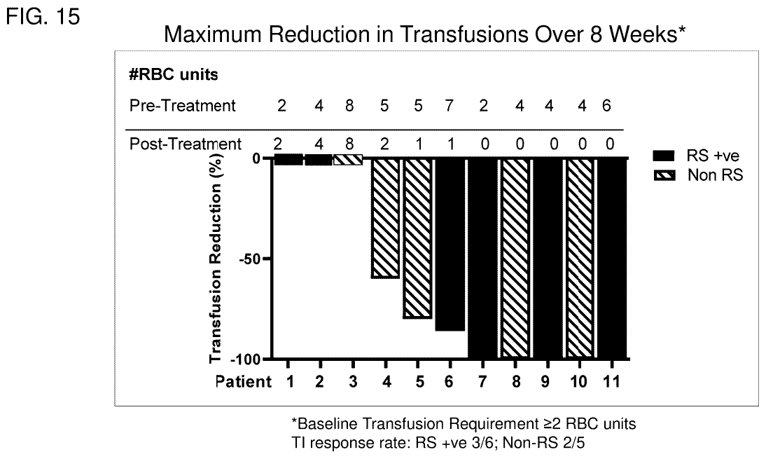

Of the evaluable patients, 43.8% (n= 7/16) achieved HI-E over an eight-week period, and 45.5% (n= 5/11) of the transfused patients receiving ≥2 RBC units at baseline achieved TI for at least eight weeks. The TI response rate was 3/6 (50%) for RS+ patients and ⅖ (50%) for non-RS patients. The majority of transfused subjects ( 9/11, 82%) were HTB and the reduction in transfusion burden over 8 weeks can be seen in FIG. 15.

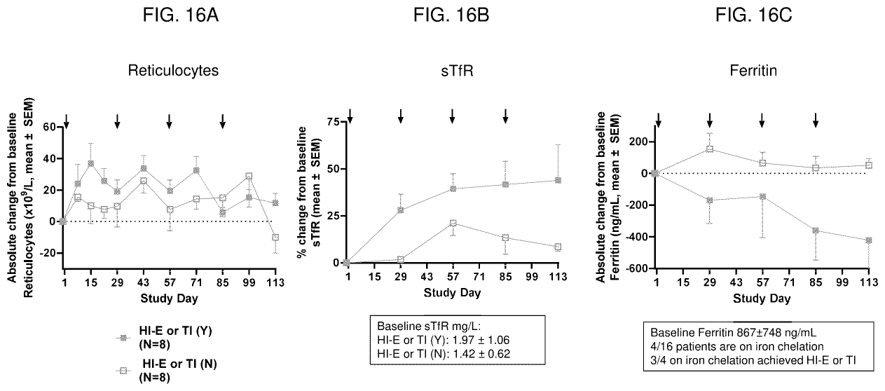

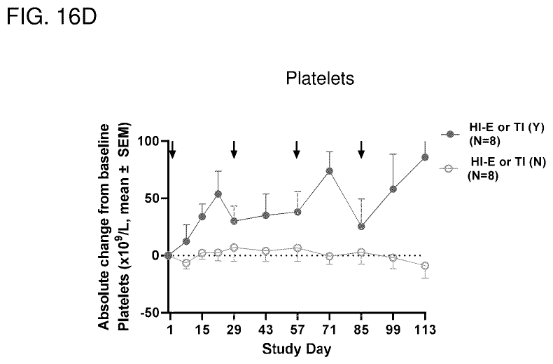

ActRIIA/B-hFc treatment demonstrated improvement in erythropoiesis and thrombopoiesis in both RS+ and non-RS MDS patients. Increases in reticulocytes, increases in serum soluble transferrin receptor (sTfR), and decreases in serum ferritin were observed in patients achieving HI-E or TI endpoints (FIGS. 16A-16C). Induction of sTfR and decreases in ferritin were progressive with treatment. Increases in reticulocytes and induction of sTfR were also observed to a lesser degree in patients who had not achieved HI-E or TI. Sustained increases in platelets were observed in patients achieving HI-E or TI endpoints (FIG. 16D), supporting the role of ActRIIA/B-hFc in inducing thrombopoiesis. No thrombocytosis or thrombotic events were observed.

In summary, ActRIIA/B-hFc has been generally well-tolerated up to the 3.75 mg/kg q4W regimen in this ongoing study. HI-E (43.8%) and transfusion independence (45.5%) were achieved in both non-RS and RS+ participants, with clinically meaningful reductions in transfusion burden and transfusion independence observed in both non-RS and RS+ patients. These data suggest that ActRIIA/B-hFc can potentially promote erythropoiesis and thrombopoiesis in patients with ineffective hematopoiesis. The observed increases in reticulocytes and soluble transferrin receptor and observed decreases in serum ferritin suggest that administration of ActRIIA/B-hFc is potentially associated with increased erythropoiesis, with a broader effect on hematopoiesis being suggested by the increase in platelets.

### Example 4—Effect of ActRIIA/B-hFc on Anemia in Patients with Very Low, Low, or Intermediate Risk Myelodysplastic Syndromes

A fourth assessment was performed on the subjects from the study described in Example 1 once additional participants had been enrolled and study duration had increased. As of the date of the fourth assessment, 31 patients in Cohorts 1-5 of the study had received at least one dose of ActRIIA/B-hFc. ActRIIA/B-hFc was observed to be generally well-tolerated as of the fourth assessment date in these 31 patients. No drug-related serious adverse events or dose-limiting toxicities were reported. The most commonly reported treatment-emergent adverse events were dyspnea, fatigue, anemia, diarrhea, and nausea. Treatment-related adverse events were reported in four patients, which were mild or moderate in severity, and did not lead to dose modification or treatment discontinuation (Grade 1: headache, pain in extremity, abdominal pain; Grade 2: rash, diarrhea, nausea, peripheral edema). The treatment-related AE of maculopapular rash was reported in one patient, after the patient's first dose, and resolved without recurrence following subsequent doses. No patients developed high-risk MDS or acute myeloid leukemia. Two patients withdrew from the trial prior to completing eight weeks of treatment with ActRIIA/B-hFc, one due to death deemed unrelated to study drug and one due to withdrawn consent.

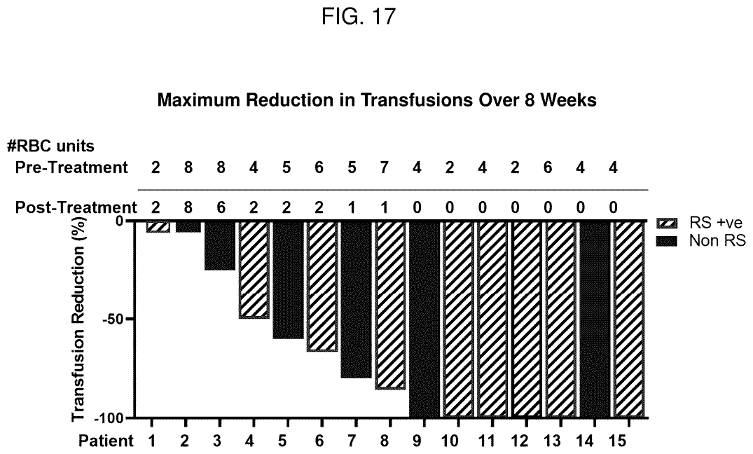

Twenty-two patients in Cohorts 1-4 had completed eight weeks of treatment and evaluation as of the fourth assessment date (“the evaluable patients”). The 22 evaluable patients were made up of five non-transfused patients (NT, subjects who received 0 RBC units in the 8 weeks prior to treatment with ActRIIA/B-hFc and had a hemoglobin level of <10 g/dL), five transfused patients with a low transfusion burden (LTB, subjects who received 1-3 RBC units in the 8 weeks prior to treatment with ActRIIA/B-hFc and had a hemoglobin level of <10 g/dL), and 12 transfused patients with a high transfusion burden (HTB, subjects who received 4 or more RBC units in the 8 weeks prior to treatment with ActRIIA/B-hFc). Two of the transfused LTB patients required <2 red blood cell units at baseline. Of the 17 transfused LTB and HTB patients, eight did not have ring sideroblasts (non-RS) and nine had ring sideroblasts (RS positive). As of the fourth assessment date, 50% (n= 11/22) of the evaluable patients achieved an overall erythroid response, which is defined as meeting one of the following two endpoints:
IWG 2006 Hematological improvement-erythroid (HI-E), which is defined as either:
   a ≥1.5 g/dL increase in hemoglobin for eight weeks in LTB and NT patients; or
   a reduction by ≥4 red blood cell (RBC) units transfused during any eight-week period during the trial, compared with the eight-week period prior to Cycle 1, Day 1 in HTB patients.
Transfusion independence (TI) for at least eight weeks in transfusion-dependent patients who required ≥2 RBC units transfused at baseline.
   Additional data from the evaluable patients in Cohorts 1-4 of the study, as of the fourth assessment date, include:
40.9% (n= 9/22) of the evaluable population achieved HI-E over an eight-week period.
46.7% (n= 7/15) of the transfused patients receiving ≥2 red blood cell units at baseline achieved TI for at least eight weeks. Of these patients, 55.6% (n= 5/9) were RS positive and 33.3% (n= 2/6) were non-RS. These data are shown in FIG. 17.

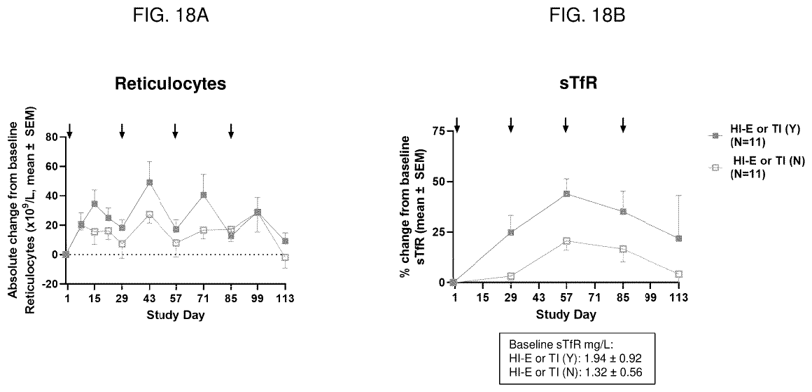

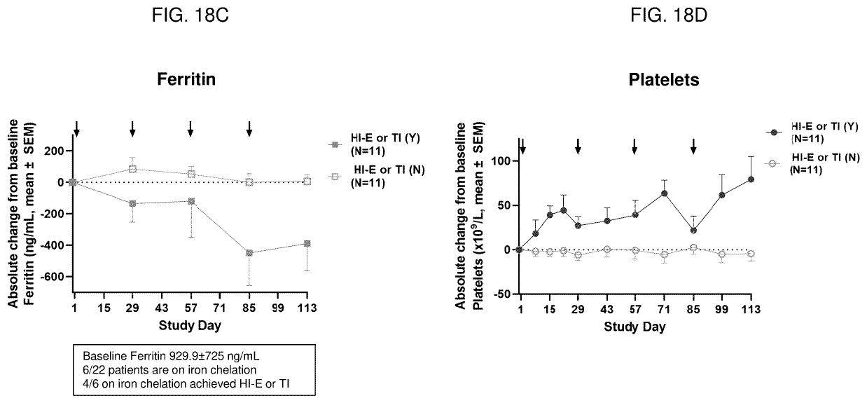

ActRIIA/B-hFc treatment demonstrated improvement in erythropoiesis and thrombopoiesis in patients in Cohorts 1-4. Increases in reticulocytes, increases in serum soluble transferrin receptor (sTfR), and decreases in serum ferritin were observed in patients achieving HI-E or TI endpoints (FIGS. 18A-18C), supporting the role of ActRIIA/B-hFc in upregulating erythropoiesis. Sustained increases in platelets were observed in patients achieving HI-E or TI endpoints (FIG. 18D), supporting the role of ActRIIA/B-hFc in upregulating thrombopoiesis. No patients required dose reduction due to thrombocytosis.

In summary, clinically meaningful reductions in transfusion burden and transfusion independence were observed in both RS+ and non-RS and patients as of the fourth assessment date. Dose levels as of the fourth assessment date were generally well tolerated. Increases in hematological parameters were also observed in both RS+ and non-RS patients, including increases in reticulocytes, hemoglobin, and platelets. The observed increases in reticulocytes and soluble transferrin receptor and observed decreases in serum ferritin suggest that administration of ActRIIA/B-hFc is potentially associated with increased erythropoiesis, with a broader effect on hematopoiesis being suggested by the increase in platelets.

### Example 5—Effect of ActRIIA/B-hFc on Anemia in Patients with Very Low, Low, or Intermediate Risk Myelodysplastic Syndromes

A fifth assessment was performed on the subjects from the study described in Example 1 once study duration had increased. Baseline characteristics for the 31 participants enrolled in the study are provided in Table 26, below. Approximately 87% of these patients presented with MLD at diagnosis and most were transfusion-dependent (approximately 84%). These participants included RS+ and non-RS patients at an approximately 1:1 ratio for each dose level with varying transfusion burdens (NT, LTB, and HTB), which were derived over an eight-week screening window at baseline, with the majority of participants being HTB. In addition, 58% of patients were HTB with elevated serum ferritin.

TABLE 26
Baseline Characteristics

| | ActRIIA/B-hFc Dose Level (mg/kg) | | | | | | |
|---|---|---|---|---|---|---|---|
| | 0.75 | 1.5 | 2.5 | 3.75 | 5 | All | |
| | (N = 6) | (N = 6) | (N = 6) | (N = 6) | (N = 7) | (N = 31) | |
| Age, Mean (range) | 75.5 | 68.3 | 72.0 | 73.3 | 76.7 | 73.3 | (55-88) |
| Female, n (%) | 5 | 1 | 4 | 2 | 0 | 12 | (38.7%) |
| Years since MDS diagnosis, Mean | 2.68 | 2.16 | 0.85 | 2.73 | 7.4 | 2.61 | (0.2-8.6) |
| (range) | | | | | | | |
| RS positive | 3 | 3 | 3 | 4 | 4 | 17 | (54.8%) |
| Transfusion Burden | | | | | | | |
| Non-Transfused (NT), 0 units | 3 | 0 | 1 | 1 | 0 | 5 | (16.1%) |
| Low Transfusion Burden (LTB), <4 units | 2 | 0 | 1 | 2 | 3 | 8 | (25.8%) |
| High Transfusion Burden (HTB), ≥4 units | 1 | 6 | 4 | 3 | 4 | 18 | (58.1%) |
| WHO MDS Classification, n (%) | | | | | | | |
| MDS-MLD | 3 | 3 | 3 | 1 | 3 | 13 | (41.9%) |
| MDS-RS-MLD | 2 | 2 | 3 | 4 | 3 | 14 | (45.2%) |
| MDS-RS-SLD | 0 | 1 | 0 | 0 | 0 | 1 | (3.2%) |
| MDS with isolated del(5q) | 1 | 0 | 0 | 0 | 0 | 1 | (3.2%) |
| N/A | 0 | 0 | 0 | 1 | 1 | 2 | (6.5%) |
| Prior ESA Therapy, n (%) | 0 | 0 | 2 | 1 | 0 | 3 | (9.7%) |
| Iron chelator, n (%) | 0 | 2 | 2 | 2 | 1 | 7 | (22.6%) |
| Efficacy Evaluable Patients*, n (%) | 6 | 4 | 6 | 6 | 5 | 27 | (87.1%) |
| Efficacy Evaluable HTB Patients**, n (%) | 1 | 4 | 4 | 3 | 4 | 16 | (88.9%) |

*Patients with at least 8 weeks of post-treatment HGB and transfusion assessments were defined as efficacy evaluable.
**Percentage based on all HTB patients.

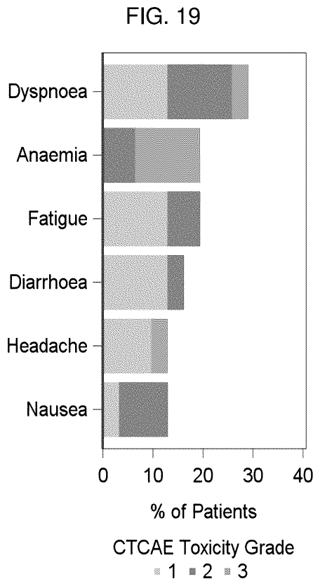

As of the third assessment date, 31 patients in Cohorts 1-5 had received at least one dose of ActRIIA/B-hFc. ActRIIA/B-hFc was observed to be generally well-tolerated in the 31 patients in Cohorts 1-5 who had received at least one dose of ActRIIA/B-hFc at all dose levels administered. No drug-related serious adverse events or dose-limiting toxicities were reported, and no patients progressed to high-risk MDS or AML. The most commonly reported treatment-emergent adverse events (≥10%) were dyspnea, fatigue, anemia, diarrhea, headache and nausea. Treatment-related adverse events were reported in five patients, which were mild or moderate in severity. Ten patients experienced SAEs, but none were drug related. Four patients withdrew from the study prior to completing treatment with ActRIIA/B-hFc, one due to death deemed unrelated to study drug (due to obesity-associated heart disease per autopsy report), one due to withdrawn consent, and two due to unrelated treatment-emergent adverse events; none required dose modification. Safety data from patients who received at least one dose of ActRIIA/B-hFc in Cohorts 1-5 are provided below in Table 27. Treatment-emergent AEs of ≥10% frequency are shown in FIG. 19.

TABLE 27
Number and Percentage of Patients with Treatment Emergent
Adverse Event as of Fifth Assessment Date

| | ActRIIA/B-hFc Dose Level (mg/kg) | | | | | | | | | |
|---|---|---|---|---|---|---|---|---|---|---|
| | 0.75 | | 1.5 | | 2.5 | | 3.75 | 5 | All | |
| n (%) | (N = 6) | | (N = 6) | | (N = 6) | | (N = 6) | (N = 7) | (N = 31) | |
| TEAE | 6 | (100) | 6 | (100) | 6 | (100) | 5 (83.3) | 6 (85.7) | 29 | (93.5) |
| Related TEAE | 1 | (16.7) | 0 | | 1 | (16.7) | 1 (16.7) | 2 (28.6) | 5 | (16.1) |
| Grade ≥3 | 1 | (16.7) | 1 | (16.7) | 3 | (50.0) | 2 (33.3) | 5 (71.4) | 12 | (38.7) |
| SAE | 1 | (16.7) | 2 | (33.3) | 1 | (16.7) | 2 (33.3) | 4 (57.1) | 10 | (32.3) |

| TEAE requiring dose | 0 | 0 | | 0 | 1 (16.7) | 1 (14.3) | 2 | (6.5) |
|---|---|---|---|---|---|---|---|---|
| modification | | | | | | | | |
| Death | 0 | 1 | (16.7) | 0 | 0 | 0 | 1 | (3.2) |

Twenty-seven patients in Cohorts 1-5 had completed at least eight weeks of treatment and evaluation as of the fifth assessment date (the “evaluable patients”). Of the 27 evaluable patients, five were non-transfused (NT, subjects who received 0 RBC units in the 8 weeks prior to treatment with ActRIIA/B-hFc and had a hemoglobin level of ≤10 g/dL), six were transfused subjects with a low transfusion burden (LTB, subjects who received 1-3 RBC units in the 8 weeks prior to treatment with ActRIIA/B-hFc and had a hemoglobin level of $9 g/dL), and 16 were transfused subjects with a high transfusion burden (HTB, subjects who received 4 or more RBC units in the 8 weeks prior to treatment with ActRIIA/B-hFc and had a hemoglobin level of $9 g/dL). Two of the transfused LTB patients required ≤2 red blood cell (“RBC”) units at baseline. Of the 20 transfused subjects with a LTB or HTB that required ≥2 RBC units at baseline, eight were non-RS and 12 were RS+. As of the fifth assessment date, 51.9% (n= 14/27) of the evaluable patients achieved an overall erythroid response, which is defined as meeting one of the following two endpoints:
IWG 2006 Hematological improvement-erythroid (HI-E), which is defined as either:
   a ≥1.5 g/dL increase in hemoglobin for eight weeks in LTB and NT patients; or
   a reduction by ≥4 red blood cell (RBC) units transfused during any eight-week period during the trial, compared with the eight-week period prior to Cycle 1, Day 1 in HTB patients.
Transfusion independence (TI) for at least eight weeks in transfusion-dependent patients who required ≥2 RBC units transfused at baseline.
   Further, when focusing on HTB patients alone, 11/16 or 68.75% achieved an overall erythroid response. These data are summarized in Table 28, below.

TABLE 28
Efficacy Summary of Eight-Week Endpoints
Achieved in MDS Patients

| | Response Rate, n/m (%) | | | |
|---|---|---|---|---|
| | All evaluable | | HTB evaluable | |
| Response Summary | patients | | patients | |
| Overall Erythroid Response (HI-E or | 14/27 | (51.8%) | 11/16 | (68.8%) |
| TI) | | | | |
| IWG 2006 HI-E | 12/26 | (46.2%) | 11/16 | (68.8%) |
| Transfusion independence (TI*) | 9/20 | (45%) | 7/16 | (43.8%) |
| RS+ | 6/12 | (50%) | 4/9 | (44.4%) |
| Non-RS | 3/8 | (37.5%) | 3/7 | (42.9%) |

*Baseline Transfusion Requirement ≥2 RBC units
n = responders in each category;
m = 8-week evaluable population as of fifth assessment date

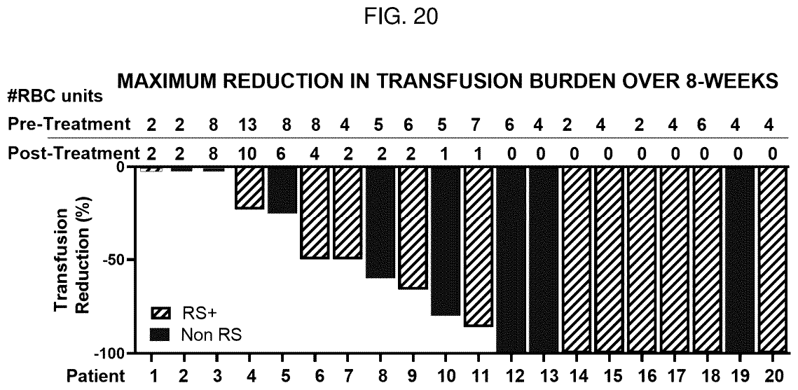

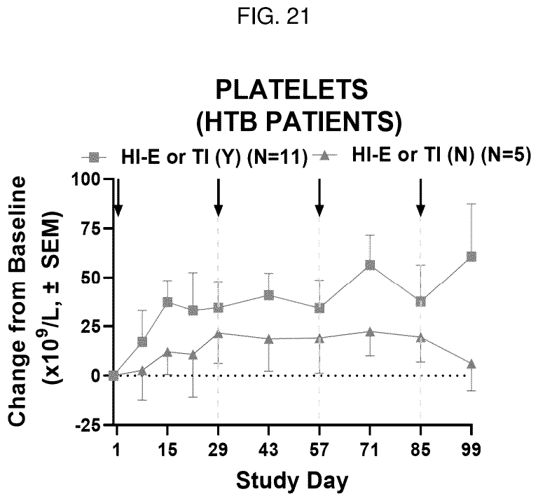

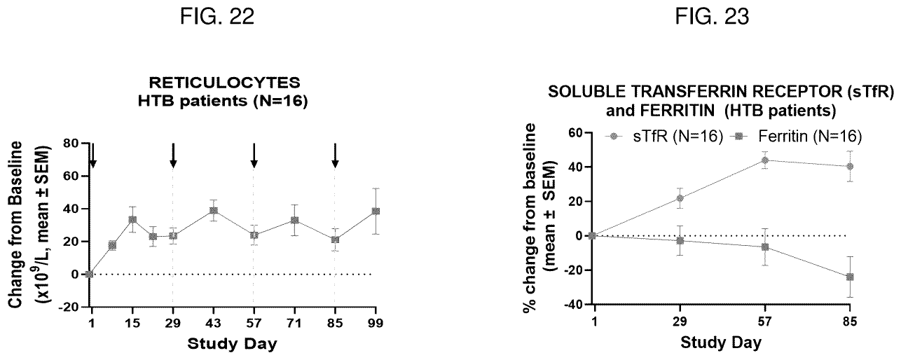

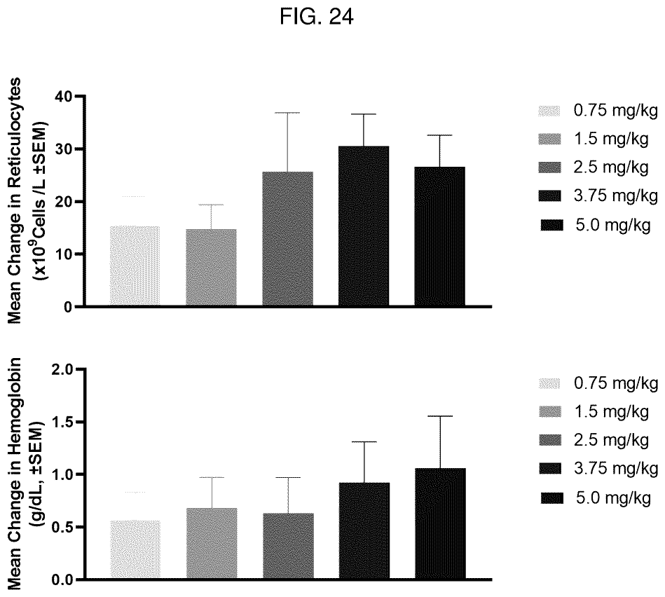

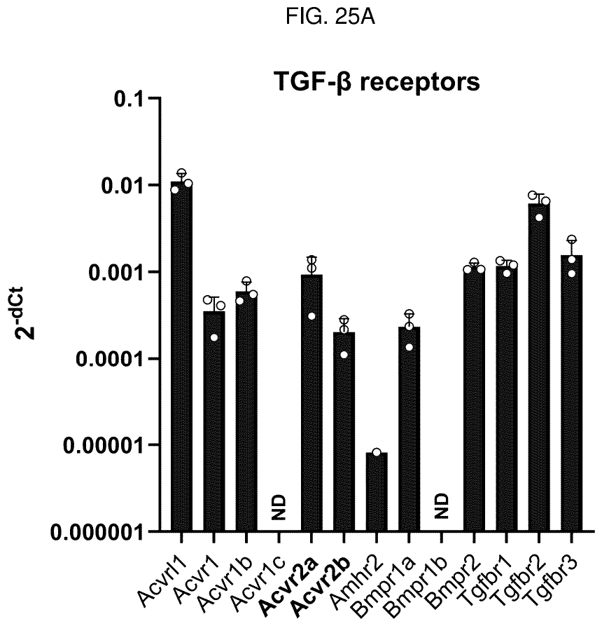

Of the evaluable patients, 46.2% (n= 12/26) achieved HI-E over an eight-week period, and 45.0% (n= 9/20) of the transfused patients receiving ≥2 RBC units at baseline achieved TI for at least eight weeks. Of these 20 patients, 12 were RS+ and eight were non-RS, with 50.0% (n= 6/12) of the RS+ patients achieving TI for at least eight weeks and 37.5% (n=⅜) of the non-RS patients achieving TI for at least eight weeks. In addition, 43.8% (n= 7/16) of the HTB patients achieved TI for at least 8 weeks. FIG. 20 shows the maximum observed transfusion reduction with ActRIIA/B-hFc treatment over an 8-week period in transfusion dependent patients with a transfusion burden ≥2 RBC units transfused at baseline (n=20). HI-E and TI were achieved in both RS+ and non-RS transfusion dependent patients, including in 7/16 HTB patients. Two out of four ( 2/4) LTB patients requiring ≥2 RBC units transfused at baseline also achieved TI. In addition, sustained increases in platelets were observed in HTB patients achieving HI-E or TI (FIG. 21). The maximum increase in platelets from baseline in HTB patients achieving HI-E or TI was 96.6×109/L (mean), range 13.8-234.8×109/L. No patients required dose reduction due to thrombocytosis. Increases in reticulocytes were observed across all enrolled HTB participants regardless of response (FIG. 22) along with decreases in serum ferritin and increases in serum soluble transferrin receptor levels (FIG. 23), suggesting an improvement in erythropoiesis. The mean maximum increase in reticulocytes from baseline in HTB patients was 62.9×109/L, range 15.7-145.8×109/L. Serum ferritin was elevated (mean baseline serum ferritin was 1359.2 ng/ml, range 230.5 to 5829.1 ng/ml) in HTB patients, indicative of transfusion-related iron overload. Following three months of treatment, patients treated with ActRIIA/B-hFc had a 29% reduction in serum ferritin (mean maximum reduction in ferritin was 29.1%, range 0 to 92%). The mean maximum sTfR increase was 52.9%, range 9.9-116.4%. FIG. 24 shows dose related changes in hematological parameters (mean change in reticulocytes and hemoglobin) for all patients over the first eight weeks of dosing. Both reticulocytes and hemoglobin increased with increasing doses of ActRIIA/B-hFc.

In summary, changes in both erythroid parameters and platelets were observed in participants treated with ActRIIA/B-hFc. Erythroid responses have been observed in both RS+ and non-RS MDS patients treated with ActRIIA/B-hFc across varying transfusion burdens, with 44% of HTB patients achieving TI during this 3-month treatment study. Reductions in serum ferritin were also observed in HTB participants. The observed effects of ActRIIA/B-hFc on reticulocytes, soluble transferrin receptor, and platelets support the proposed ActRIIA/B-hFc mechanism of increasing hematopoiesis.

### Example 6—Effect of ActRIIA/B-mFc on Platelets

Eleven-week-old C57Bl/6 mice were dosed with either TBS (Vehicle) or ActRIIA/B-mFc (10 mg/kg) via intraperitoneal (IP) administration. Twelve hours post-dose whole blood was sampled, and platelet counts were determined using a veterinary hematology analyzer (Heska Element HT5). Mice were then euthanized, and bone marrow extracted from the femurs. Bone marrow cells were stained with antibodies against Lineage (Perc-Cy5), sca1 (BV525), cKit (Alexa750), CD41 (APC) and CD150 (Pcy7) and analyzed on flow cytometer (Cytoflex, Beckman coulter). Megakaryocyte progenitors were gated on Lin−; sca1−; ckit+; CD150+; CD41+ cells.

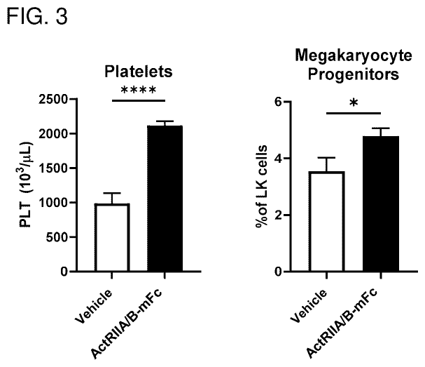

FIG. 3 shows the effect of ActRIIA/B-mFc on thrombopoiesis. A single dose of ActRIIA/B-mFc increased circulating platelet numbers and bone marrow megakaryocyte progenitors within 12 hours post-dose. The timing of the effect on platelets is suggestive of a direct effect of ActRIIA/B-mFc on the terminal maturation of proplatelets to platelets and the megakaryocyte progenitor data demonstrate that that ActRIIA/B-mFc affects earlier stages of the platelet formation process. Data are represented as mean±SEM. Statistical analysis was performed with a Student T-Test; *p<0.05; **p<0.01; ***p<0.001; ****p<0.0001.

### Example 7—Effect of ActRIIA/B-mFc on Megakaryocyte Differentiation and Maturation

Eleven-week-old C57Bl/6 mice were dosed with either TBS (Vehicle) or ActRIIA/B-mFc (10 mg/kg) via IP administration. Twelve and twenty-four hour later mice were euthanized, and bone marrow extracted from the femurs. Bone marrow cells were fixed in ethanol following by a staining with Propidium Iodide (PI) and anti CD41 (FITC, Efferet) antibody in parallel with RNAse treatment. Samples were analyzed by flow cytometer (Cytoflex, Beckman coulter). Ploidy of CD41+ nucleated cells (PI+ cells) was analyzed. Graphs represent % cells at each ploidy stage. For 12HR N=3 and for 24HR N=2. For CD41+ cells at 12HR time point a t-test was performed for statistical analysis. Data are presented as mean±SEM.

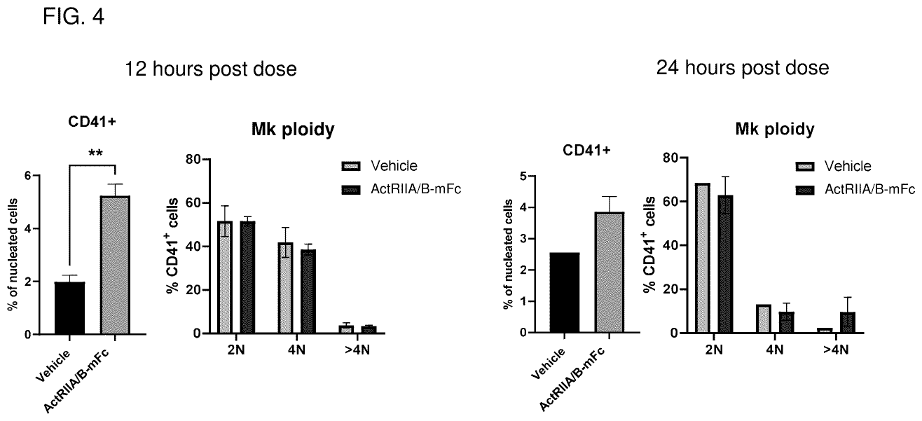

FIG. 4 shows that ActRIIA/B-mFc treatment exhibits a direct effect on megakaryocyte differentiation and maturation as shown by an increase in the number of CD41+ megakaryocyte progenitors at 12 hours after treatment and an increase in number of polyploid megakaryocytes by 24 hours. These data indicate an early effect on progenitors of the thrombopoiesis pathway, indicate that ActRIIA/B-mFc increased differentiation of megakaryocyte precursors toward later stages of maturation, and demonstrate that ActRIIA/B-mFc treatment results in a greater number of megakaryocytes that are potentially primed for more proplatelet production.

### Example 8—Effect of ActRIIA/B-mFc on Platelet Recovery Following Depletion

Twelve-week-old mice were treated with either anti-GP1bα (0.08 mg/kg, Efferet) or IgG control. At day 4 after treatment, the anti-GP1bα treated group was further divided to receive either vehicle or ActRIIA/B-mFc (7.5 mg/kg) treatment. Platelets were measured at indicated time points post anti-GP1bα dosing. At day 10 post-treatment, mice were euthanized, and bone marrow cells were harvested. Bone marrow cells were fixed in ice-cold 100% ethanol and stained with Propidium Iodide (PI) (200 μg/mL, Sigma-Aldrich) and anti-CD41 (FITC conjugated, Emfret Analytics) antibody in parallel with RNase treatment (2 mg/ml, Invitrogen). Samples were analyzed by flow cytometer (Cytoflex, Beckman coulter) and % CD41+ nucleated cells (PI+ cells) was measured.

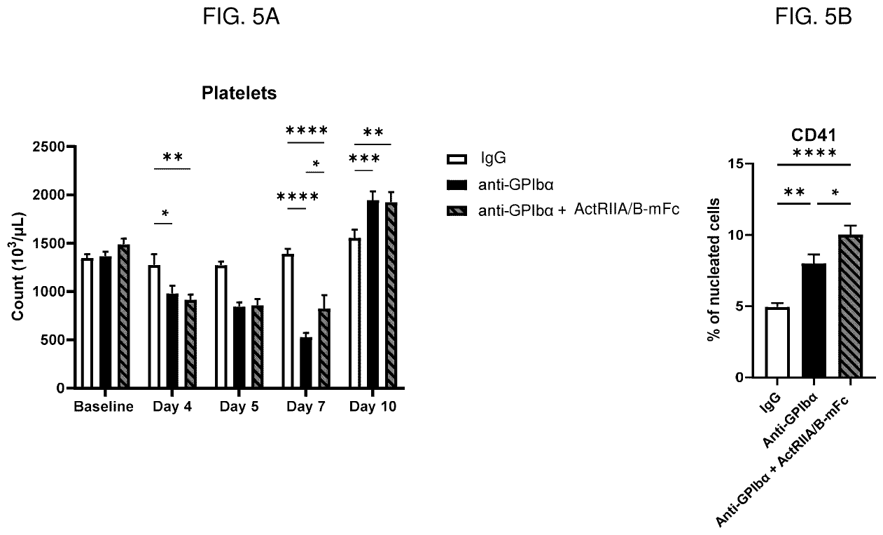

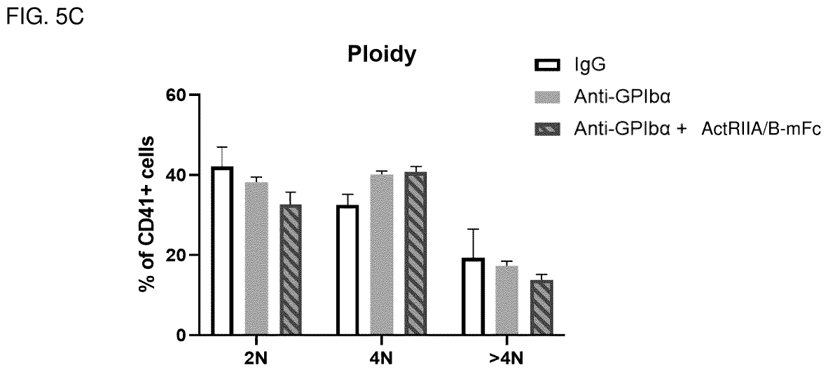

As shown in FIG. 5A, mice treated with ActRIIA/B-mFc exhibited accelerated recovery of platelet numbers following platelet depletion compared to vehicle treated mice in a mouse model of immune thrombocytopenia. These data suggest that ActRIIA/B-mFc could potentially promote faster recovery from thrombocytopenia. Additionally, as shown in FIGS. 5B-5C, there was a 25% increase in the number of CD41+ megakaryocyte progenitors in the bone marrow of the ActRIIA/B-mFc-treated group compared to the vehicle-treated group at day 10 after platelet depletion and a higher 4N ploidy level, suggesting that under acute thrombocytopenia, ActRIIA/B-mFc treatment promotes differentiation of megakaryocytes, potentially by accelerating maturation of megakaryocyte precursors, and contributes to accelerated recovery in mice. For platelet data, statistical was analysis conducted with repeat measures mixed effect modeling. Individual comparisons shown are from a Tukey post-test. For CD41 data, statistical analysis was performed with 1-Way ANOVA and individual comparisons calculated using a Tukey post-test. *p<0.05; **p<0.01; ***p<0.001; ****p<0.0001. N=9/group.

### Example 9—Effect of a Single Dose of ActRIIA/B-mFc on Platelet Numbers

Eleven-week-old C57Bl/6 mice were given a single dose of either TBS (vehicle) or ActRIIA/B-mFc (10 mg/kg) via subcutaneous administration. Separate cohorts of mice from both dosing groups were sampled for whole blood at study day 37, 51 and 85, and platelet counts were determined using a veterinary hematology analyzer (Heska Element HT5).

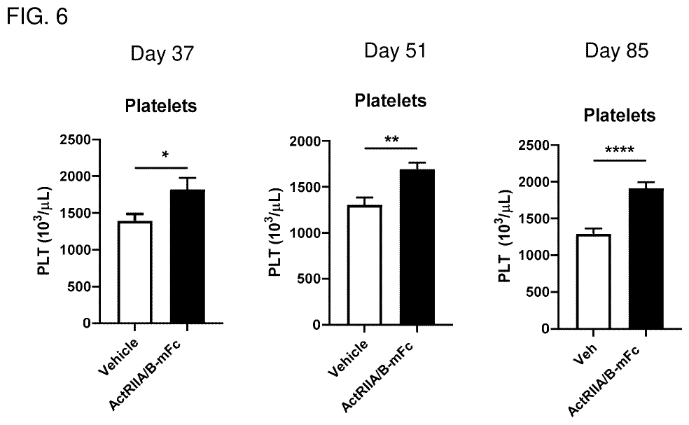

As shown in FIG. 6, a single dose of ActRIIA/B-mFc resulted in increased circulating platelets at day 37, 51, and 85. Data are shown as mean±SEM. Statistical analysis was performed using a Student T-Test; *p≤0.05; **p≤0.01; ***p≤0.001; ****p≤0.0001.

### Example 10—Effect of ActRIIA/B-mFc on Megakaryocyte Precursors Ex-Vivo

Bone marrow cells from 11-week-old C57Bl/6 mice were isolated and treated with Activin A (5 mg/kg), ActRIIA/B-mFc (10 mg/kg), or a combination of both for six days. Cells were harvested after six days and analyzed using flow cytometry (N=2).

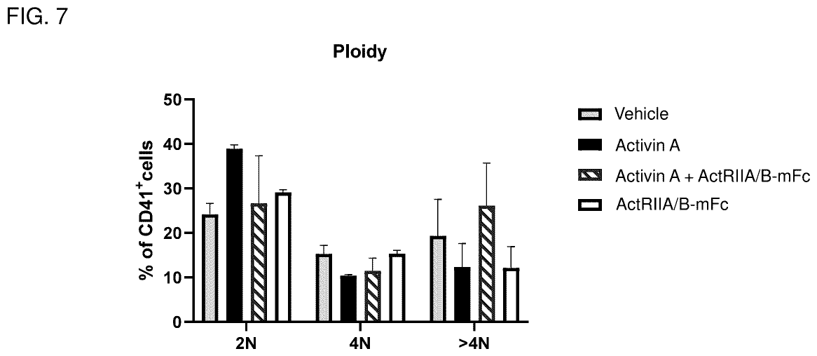

As shown in FIG. 7, ex vivo activin A treatment increased 2N ploidy levels and reduced the extent of polyploidy (decreased higher ploidy levels), an indication that activin A was acting to prevent maturation of these cells. Ex-vivo treatment with ActRIIA/B-mFc reversed activin-mediated changes in megakaryocyte precursors, indicating that ActRIIA/B-mFc inhibited the effects of Activin A on ploidy. Higher polyploid levels appeared in the Activin A+ ActRIIA/B-mFc treated group compared to the Activin A group. Data are represented as mean±SEM.

### Example 11—Effect of an Anti-Activin a Antibody on Platelet Numbers

Ten-week-old C57Bl/6 male mice were dosed with either TBS (vehicle), anti-activin A antibody (described in WO2008031061A2, 5 mg/kg), or ActRIIA/B-mFc (10 mg/kg) via intraperitoneal administration. Twenty-four hours post-dose whole blood was sampled, and platelet counts were determined using a veterinary hematology analyzer (Hematrue).

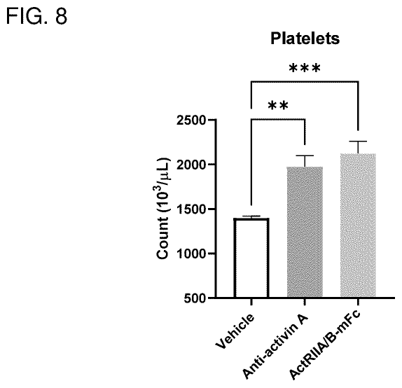

As shown in FIG. 8, treatment with an anti-activin A antibody and with ActRIIA/B-mFc increased platelets in wild-type mice. Observed increases in platelets with the anti-activin A antibody suggests that inhibition of activin A may be at least partly responsible for the increase in platelet levels observed with ActRIIA/B-mFc. Data are represented as mean±SEM. Data were analyzed using Prism 9 (GraphPad Software, San Diego, CA, USA) using one-way ANOVA with Fisher's LSD. *p<0.05, **p<0.01, ***p<0.001, ****p<0.0001.

### Example 12—Effect of ActRIIA/B-mFc on Platelets, Red Blood Cell Parameters, Spleen Weight, and Immune Cells in a TPO^{high} Model of Myelofibrosis

The TPO^{high} model of myelofibrosis induces a myelofibrotic-like pathology through elevated exposure to thrombopoietin, the native endocrine inducer of megakaryocyte progenitor proliferation and development. Seven-week-old C57Bl/6 albino mice (B6(Cg)-Tyr, Jackson Laboratory) were teil-vein injected with 0.75 mg/kg thrombopoietin (TPO) expressing plasmid cloned into pLEV113 plasmid (Lake Pharma). The injection was done in hydrodynamic approach in which 100 ml/kg volume is injected in a short period of time (6-10 seconds). On day 3 after TPO injection mice were divided into 2 groups receiving either vehicle (TBS) or ActRIIA/B-mFc (7.5 mg/kg), twice weekly. Mice were sacrificed on day 14 after TPO injection. Hematological parameters were measured using the Heska Element HT5 veterinary hematology analyzer.

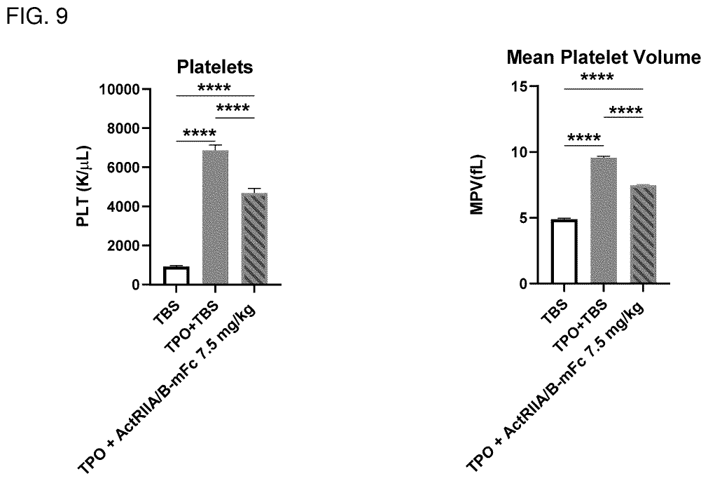

As shown in FIG. 9, TPO HDI increased platelet number and volume. Treatment with ActRIIA/B-mFc was associated with a significant attenuation in the expansion of platelets. High platelet levels are associated with thrombocythemia which can lead to secondary myelofibrosis. These data may suggest that ActRIIA/B-mFc is rebalancing the number of cells committed to the megakaryocyte lineage. N=10-12 mice/group. Results are presented as mean±SEM. Statistical analysis was performed by one-way ANOVA. ****=p<0.0001.

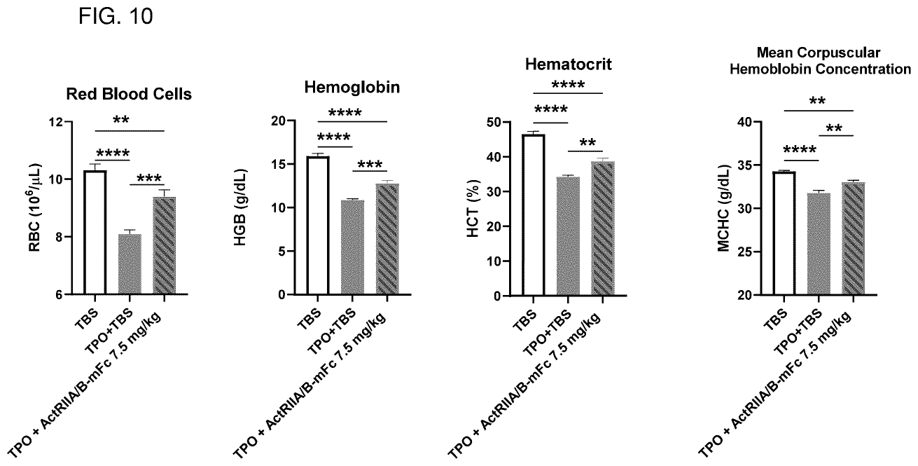

Data confirmed that the TPO^{high} myelofibrosis model was anemic after 14 days of TPO overexpression (FIG. 10). Treatment with ActRIIA/B-mFc was associated with a significant improvement in RBC metrics and appeared to reduce the development of anemia in this model. N=10-12 mice/group. Results are presented as mean±SEM. Statistical analysis was performed by one-way ANOVA. **=p<0.01; ***=p<0.001; ****=p<0.0001.

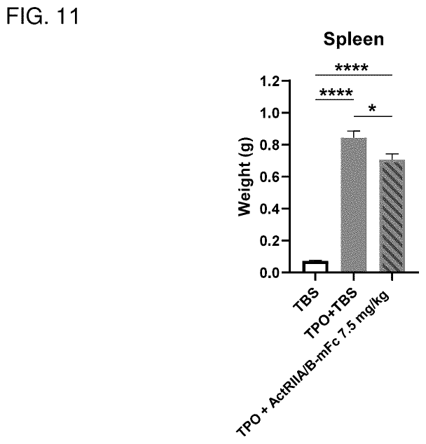

The expansion in megakaryocyte growth and proliferation in this model reduces the capacity of bone marrow for hematopoiesis, inducing compensatory extra medullary hematopoiesis in the liver and spleen (FIG. 11). These data show a significant reduction in splenomegaly in mice treated with ActRIIA/B-mFc, indicating reduced splenic extra medullary hematopoiesis, likely due to a reduced requirement for this compensatory process. N=10-12 mice/group. Results are presented as mean±SEM. Statistical analysis was performed by one-way ANOVA. *=p<0.05; ****=p<0.0001.

Finally, as shown in FIG. 12, TPO HDI led to a rapid increase in white blood cells, neutrophils, and lymphocytes and treatment with ActRIIA/B-mFc reduced the TPO-mediated increase in white blood cells and lymphocytes. TPO signals through the JAK2 pathway. The JAK2 pathway is proliferative and activating mutations are associated with neoplastic syndromes and a predisposition for the development of myeloid leukemias. N=10-12 mice/group. Results are presented as mean±SEM. Statistical analysis was performed by one-way ANOVA. ***=p<0.001; ****=p<0.0001.

### Example 13—Megakaryocyte Precursor Cells Express Activins, GDFs, BMPs and TGF-β Ligands and their Cognate Receptors

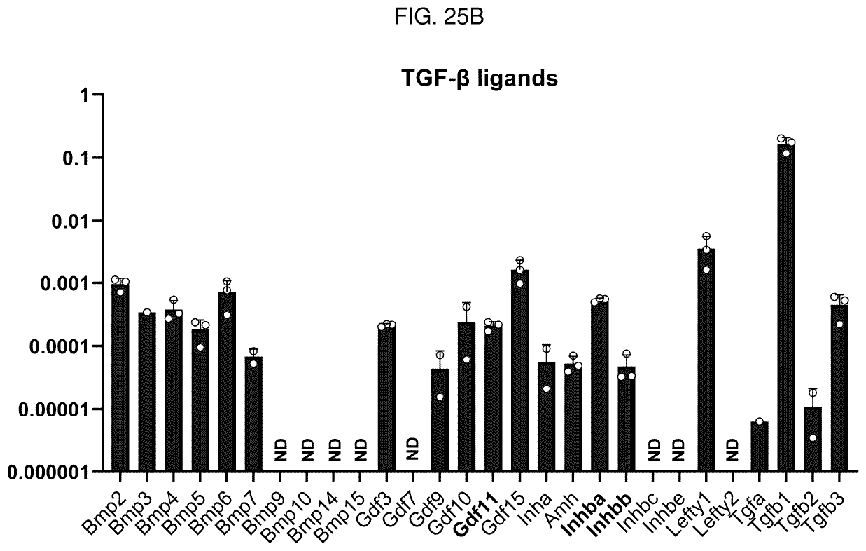

Bone marrow from three untreated mice was pooled and selected for megakaryocyte marker CD41. Once cells underwent positive selection using Rat anti-mouse CD41 (clone-MWReg30), RNA was extracted via Zymo Research Direct-zol RNA MicroPrep Kit. Total RNA was then converted to cDNA (QuantiTect Reverse Transcription Kit) and at least 20 ng per well was added to a mouse specific TGF-β family pathway TaqMan gene expression array (ThermoFisher Custom Gene Array). Quantitative real time PCR was performed, and results were plotted using 2\hat{ }delta Ct values. Housekeeping genes including 18s, Gusb, Gapdh, Actb, and Ubc were used to normalize TGF-β gene expression. The results are shown in FIGS. 25A-25B and represent 3 independent experimental repeats. As shown in FIGS. 25A-25B, murine bone marrow megakaryocyte precursors expressed activins, GDFs, BMPs, and TGF-β ligands and their cognate receptors, including activin receptors IIA and IIB. These data demonstrate the capability of the TGF-β pathway to be involved in the differentiation of megakaryocytes and in normal megakaryocyte function. The gene that encodes the activin A protein, INHBA, was moderately expressed compared to other family member ligands. Receptors and ligands directly related to ActRIIA/B-mFc are in bold. ND, not detected.

### Example 14—Treatment of an MDS-Associated Cytopenia by Administration of an ActRIIA Ligand Trap Including an Extracellular ActRIIA Variant

According to the methods disclosed herein, a physician of skill in the art can treat a subject, such as a human patient, having a cytopenia (e.g., anemia and/or thrombocytopenia) due to a low, very low, or intermediate risk myelodysplastic syndrome so as to increase red blood cell count, increase hemoglobin levels, increase hematocrit, increase reticulocytes, increase the maturation and/or differentiation of erythroid progenitors increase late-stage erythroid precursor maturation, increase the number of early-stage erythroid precursors and/or progenitors, promote the progression of erythroid precursors and/or progenitors through erythropoiesis, recruit early-stage progenitors into the erythroid lineage, increase platelet levels, increase or induce megakaryocyte differentiation and/or maturation, reduce platelet progenitor accumulation, increase megakaryocyte progenitors (e.g., increase megakaryocyte progenitor renewal), and/or promote or increase platelet formation or production. The method of treatment can include diagnosing or identifying a subject as a candidate for treatment using the IPSS-R and/or measuring hemoglobin levels and platelet levels. To treat the subject, a physician of skill in the art can administer to the subject a composition containing an ActRIIA ligand trap including an extracellular ActRIIA variant (e.g., an extracellular ActRIIA variant having the sequence of any one of SEQ ID NOs: 1-72 (e.g., SEQ ID NOs: 6-72)). The composition containing the ActRIIA ligand trap including an extracellular ActRIIA variant may be administered to the subject, for example, by parenteral injection (e.g., intravenous or subcutaneous injection) to treat the MDS-associated cytopenia (e.g., anemia and/or thrombocytopenia). The ActRIIA ligand trap including an extracellular ActRIIA variant (e.g., an extracellular ActRIIA variant having the sequence of any one of SEQ ID NOs: 1-72 (e.g., SEQ ID NOs: 6-72)) is administered in a therapeutically effective amount, such as from 0.01 to 500 mg/kg (e.g., 0.01, 0.1, 0.2, 0.3, 0.325, 0.35, 0.375, 0.4, 0.5, 0.75, 1, 1.25, 1.5, 1.75, 2, 2.25, 2.5, 2.75, 3, 3.25, 3.5, 3.75, 4, 4.25, 4.5, 4.75, 5, 10, 15, 20, 25, 30, 35, 40, 45, 50, 100, 150, 200, 250, 300, 350, 400, 450, or 500 mg/kg). In some embodiments, the ActRIIA ligand trap including an extracellular ActRIIA variant is administered bimonthly, once a month, once every four weeks, once every two weeks, or at least once a week or more (e.g., 1, 2, 3, 4, 5, 6, or 7 times a week or more). The ActRIIA ligand trap including an extracellular ActRIIA variant is administered in an amount sufficient to increase red blood cell count, increase hemoglobin levels, increase hematocrit, increase reticulocytes, increase the maturation and/or differentiation of erythroid progenitors, increase late-stage erythroid precursor maturation, increase the number of early-stage erythroid precursors and/or progenitors, promote the progression of erythroid precursors and/or progenitors through erythropoiesis, recruit early-stage progenitors into the erythroid lineage, increase platelet levels, increase or induce megakaryocyte differentiation and/or maturation, reduce platelet progenitor accumulation, increase megakaryocyte progenitors, and/or promote or increase platelet formation or production.

Following administration of the composition to a patient, a practitioner of skill in the art can monitor the patient's improvement in response to the therapy by a variety of methods. For example, a physician can monitor the patient's red blood cell count, hemoglobin levels, hematocrit, or platelet count using a blood test. A finding that the patient's red blood cell count, hemoglobin levels, hematocrit, reticulocytes, and/or platelet count are increased following administration of the composition compared to test results prior to administration of the composition indicates that the patient is responding favorably to the treatment. Subsequent doses can be determined and administered as needed.

### Example 15—Treatment of an CMML-O-Associated Cytopenia by Administration of an ActRIIA Ligand Trap Including an Extracellular ActRIIA Variant

According to the methods disclosed herein, a physician of skill in the art can treat a subject, such as a human patient, having a cytopenia (e.g., anemia and/or thrombocytopenia) due to CMML-0 so as to increase red blood cell count, increase hemoglobin levels, increase hematocrit, increase reticulocytes, increase the maturation and/or differentiation of erythroid progenitors increase late-stage erythroid precursor maturation, increase the number of early-stage erythroid precursors and/or progenitors, promote the progression of erythroid precursors and/or progenitors through erythropoiesis, recruit early-stage progenitors into the erythroid lineage, increase platelet levels, increase or induce megakaryocyte differentiation and/or maturation, reduce platelet progenitor accumulation, increase megakaryocyte progenitors (e.g., increase megakaryocyte progenitor renewal), and/or promote or increase platelet formation or production. The method of treatment can include hemoglobin levels and platelet levels. To treat the subject, a physician of skill in the art can administer to the subject a composition containing an ActRIIA ligand trap including an extracellular ActRIIA variant (e.g., an extracellular ActRIIA variant having the sequence of any one of SEQ ID NOs: 1-72 (e.g., SEQ ID NOs: 6-72)). The composition containing the ActRIIA ligand trap including an extracellular ActRIIA variant may be administered to the subject, for example, by parenteral injection (e.g., intravenous or subcutaneous injection) to treat the CMML-0-associated cytopenia (e.g., anemia and/or thrombocytopenia). The ActRIIA ligand trap including an extracellular ActRIIA variant (e.g., an extracellular ActRIIA variant having the sequence of any one of SEQ ID NOs: 1-72 (e.g., SEQ ID NOs: 6-72)) is administered in a therapeutically effective amount, such as from 0.01 to 500 mg/kg (e.g., 0.01, 0.1, 0.2, 0.3, 0.325, 0.35, 0.375, 0.4, 0.5, 0.75, 1, 1.25, 1.5, 1.75, 2, 2.25, 2.5, 2.75, 3, 3.25, 3.5, 3.75, 4, 4.25, 4.5, 4.75, 5, 10, 15, 20, 25, 30, 35, 40, 45, 50, 100, 150, 200, 250, 300, 350, 400, 450, or 500 mg/kg). In some embodiments, the ActRIIA ligand trap including an extracellular ActRIIA variant is administered bimonthly, once a month, once every four weeks, once every two weeks, or at least once a week or more (e.g., 1, 2, 3, 4, 5, 6, or 7 times a week or more). The ActRIIA ligand trap including an extracellular ActRIIA variant is administered in an amount sufficient to increase red blood cell count, increase hemoglobin levels, increase hematocrit, increase reticulocytes, increase the maturation and/or differentiation of erythroid progenitors, increase late-stage erythroid precursor maturation, increase the number of early-stage erythroid precursors and/or progenitors, promote the progression of erythroid precursors and/or progenitors through erythropoiesis, recruit early-stage progenitors into the erythroid lineage, increase platelet levels, increase or induce megakaryocyte differentiation and/or maturation, reduce platelet progenitor accumulation, increase megakaryocyte progenitors, and/or promote or increase platelet formation or production.

Following administration of the composition to a patient, a practitioner of skill in the art can monitor the patient's improvement in response to the therapy by a variety of methods. For example, a physician can monitor the patient's red blood cell count, hemoglobin levels, hematocrit, or platelet count using a blood test. A finding that the patient's red blood cell count, hemoglobin levels, hematocrit, reticulocytes, and/or platelet count are increased following administration of the composition compared to test results prior to administration of the composition indicates that the patient is responding favorably to the treatment. Subsequent doses can be determined and administered as needed.

### Example 16—Treatment of Myelofibrosis by Administration of an ActRIIA Ligand Trap Containing Extracellular ActRIIA Variant

According to the methods disclosed herein, a physician of skill in the art can treat a subject, such as a human patient, with myelofibrosis (e.g., PMF, post-ET MF, and post-PV MF) and having anemia so as to increase red blood cell count, increase hemoglobin levels, increase hematocrit, decrease RBC transfusions, promote transfusion independence, reduce spleen volume, reduce osteosclerosis, reduce bone marrow fibrosis, and/or treat anemia. The method of treatment can include diagnosing or identifying a subject as a candidate for treatment by measuring hemoglobin levels. To treat the subject, a physician of skill in the art can administer to the subject a composition containing an ActRIIA ligand trap containing an extracellular ActRIIA variant (e.g., an extracellular ActRIIA variant having the sequence of any one of SEQ ID NOs: 1-72 (e.g., SEQ ID NOs: 6-72)). The composition containing the ActRIIA ligand trap containing an extracellular ActRIIA variant may be administered to the subject, for example, by parenteral injection (e.g., intravenous or subcutaneous injection). The ActRIIA ligand trap containing an extracellular ActRIIA variant (e.g., an extracellular ActRIIA variant having the sequence of any one of SEQ ID NOs: 1-72 (e.g., SEQ ID NOs: 6-72)) is administered in a therapeutically effective amount, such as from 0.01 to 500 mg/kg (e.g., 0.01, 0.1, 0.2, 0.3, 0.325, 0.35, 0.375, 0.4, 0.5, 0.75, 1, 1.25, 1.5, 1.75, 2, 2.25, 2.5, 2.75, 3, 3.25, 3.5, 3.75, 4, 4.25, 4.5, 4.75, 5, 10, 15, 20, 25, 30, 35, 40, 45, 50, 100, 150, 200, 250, 300, 350, 400, 450, or 500 mg/kg). In some embodiments, the ActRIIA ligand trap including an extracellular ActRIIA variant is administered bimonthly, once a month, once every four weeks, once every two weeks, or at least once a week or more (e.g., 1, 2, 3, 4, 5, 6, or 7 times a week or more). The ActRIIA ligand trap containing an extracellular ActRIIA variant is administered in an amount sufficient to increase red blood cell count, increase hemoglobin levels, increase hematocrit, decrease RBC transfusions, promote transfusion independence, reduce spleen volume, reduce osteosclerosis, reduce bone marrow fibrosis, and/or treat anemia.

Following administration of the composition to a patient, a practitioner of skill in the art can monitor the patient's improvement in response to the therapy by a variety of methods. For example, a physician can monitor the patient's red blood cell count, hemoglobin levels, or hematocrit using a blood test. A finding that the patient's red blood cell count, hemoglobin levels, or hematocrit are increased following administration of the composition compared to test results prior to administration of the composition indicates that the patient is responding favorably to the treatment. Subsequent doses can be determined and administered as needed.

### OTHER EMBODIMENTS

While the invention has been described in connection with specific embodiments thereof, it will be understood that it is capable of further modifications and this application is intended to cover any variations, uses, or adaptations of the invention following, in general, the principles of the invention and including such departures from the present disclosure come within known or customary practice within the art to which the invention pertains and may be applied to the essential features hereinbefore set forth.

All publications, patents, and patent applications are herein incorporated by reference in their entirety to the same extent as if each individual publication, patent, or patent application was specifically and individually indicated to be incorporated by reference in its entirety.

Other embodiments are within the following claims.

1-14. (canceled)

15. A method of treating a cytopenia in a subject having chronic myelomonocytic leukemia (CMML), the method comprising the step of administering to the subject a therapeutically effective amount of an ActRII signaling inhibitor.

16-17. (canceled)

18. The method of claim 15, wherein the CMML is CMML-0

19. A method of treating in a subject having primary myelofibrosis (PMF), post-essential thrombocythemia myelofibrosis (post-ET MF), or post-polycythemia vera myelofibrosis (post-PV MF) or treating a cytopenia in a subject having PMF, post-ET MF, or post-PV MF, the method comprising the step of administering to the subject an effective amount of an ActRII signaling inhibitor.

20-36. (canceled)

37. A method of treating a cytopenia in a subject having myelofibrosis who has discontinued treatment with a JAK inhibitor or who is ineligible for treatment with a JAK inhibitor, the method comprising the step of administering to the subject an effective amount of an ActRII signaling inhibitor.

38. The method of claim 37, wherein the subject had relapsed disease following treatment with the JAK inhibitor, is refractory to treatment with the JAK inhibitor, is intolerant to treatment with a JAK inhibitor, or no longer meets the risk/benefit ratio to continue treatment with the JAK inhibitor.

39-43. (canceled)

44. The method of claim 19, wherein the myelofibrosis is intermediate- or high-risk myelofibrosis.

45-53. (canceled)

54. The method of claim 15, wherein the cytopenia is anemia.

55. The method of claim 15, wherein the cytopenia is thrombocytopenia.

56. The method of claim 15, wherein the cytopenia is neutropenia.

57-69. (canceled)

70. The method of claim 15, wherein the ActRII signaling inhibitor is:
(a) an activin A antibody or an antigen binding fragment thereof;
(b) a myostatin antibody or an antigen binding fragment thereof;
(c) an ActRII antibody or an antigen binding fragment thereof;
(d) an ActRII ligand trap;
(e) an activin B antibody or an antigen binding fragment thereof; or
(f) a GDF-11 antibody or an antigen binding fragment thereof.

71-77. (canceled)

78. The method of claim 70, wherein the ActRII ligand trap is a polypeptide comprising an extracellular activin receptor type IIa (ActRIIa) variant, the variant having a sequence of GAILGRSETQECLX₁X₂NANWX₃X₄X₅X₆TNQTGVEX₇CX₈GX₉X₁₀X₁₁X₁₂X₁₃X₁₄HCX₁₅ATWX₁₆NISGSIEIVX₁₇X₁₈ GCX₁₉X₂₀X₂₁DX₂₂NCYDRTDCVEX₂₃X₂₄X₂₅X₂₆PX₂₇VYFCCCEGNMCNEKFSYFPEMEVTQPTS (SEQ ID NO: 1),
wherein X₁ is F or Y; X₂ is F or Y; X₃ is E or A; X₄ is K or L; X₅ is D or E; X₆ is R or A; X₇ is P or R; X₈ is Y or E; X₉ is D or E; X₁₀ is K or Q; X₁₁ is D or A; X₁₂ is K or A; X₁₃ is R or A; X₁₄ is R or L; X₁₅ is F or Y; X₁₆ is K, R, or A; X₁₇ is K, A, Y, F, or I; X₁₈ is Q or K; X₁₉ is W or A; X₂₀ is L or A; X₂₁ is D, K, R, A, F, G, M, N, or I; X₂₂ is I, F, or A; X₂₃ is K or T; X₂₄ is K or E; X₂₅ is D or E; X₂₆ is S or N; and X₂₇ is E or Q, and
wherein the variant has at least one amino acid substitution relative to a wild-type extracellular ActRIIa having the sequence of SEQ ID NO: 73.

79-84. (canceled)
